SEQUENCE LISTING (246 sequences)

SEQ ID NO: 1 - protein, 663 aa, Homo sapiens
  REGION 1..663: note = misc_feature - Human protein arginine deiminase type 4 (huPAD4; UniProt ID: Q9UM07)
MAQGTLIRVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMGPDFGYVTRGPQTGGISGLDSFGNLEVSPPVTVRGKEYPLGRILFGDSCYPSNDSRQMHQALQDFLSAQQVQAPVKLYSDWLSVGHVDEFLSFVPAPDRKGFRLLLASPRSCYKLFQEQQNEGHGEALLFEGIKKKKQQKIKNILSNKTLREHNSFVERCIDWNRELLKRELGLAESDIIDIPQLFKLKEFSKAEAFFPNMVNMLVLGKHLGIPKPFGPVINGRCCLEEKVCSLLEPLGLQCTFINDFFTYHIRHGEVHCGTNVRRKPFSFKWWNMVP

SEQ ID NO: 2 - protein, 663 aa, Homo sapiens
  REGION 1..663: note = misc_feature - Full-length PAD4 construct
MAQGTLIRVTPEQPTHAVCVLGTLTQLDICSSAPEDCTSFSINASPGVVVDIAHSPPAKKKSTGSSTWPLDPGVEVTLTMKAASGSTGDQKVQISYYGPKTPPVKALLYLTAVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMGPDFGYVTRGPQTGGISGLDSFGNLEVSPPVTVRGKEYPLGRILFGDSCYPSNDSRQMHQALQDFLSAQQVQAPVKLYSDWLSVGHVDEFLSFVPAPDRKGFRLLLASPRSCYKLFQEQQNEGHGEALLFEGIKKKKQQKIKNILSNKTLREHNSFVERCIDWNRELLKRELGLAESDIIDIPQLFKLKEFSKAEAFFPNMVNMLVLGKHLGIPKPFGPVINGRCCLEEKVCSLLEPLGLQCTFINDFFTYHIRHGEVHCGTNVRRKPFSFKWWNMVP

SEQ ID NO: 3 - protein, 684 aa, Homo sapiens
  REGION 1..684: note = misc_feature - Full-length PAD4 construct with cleavable N-terminal his tag and TMVM tag
MGSSHHHHHHSSGETVRFQGHMAQGTLIRVTPEQPTHAVCVLGTLTQLDICSSAPEDCTSFSINASPGVVVDIAHSPPAKKKSTGSSTWPLDPGVEVTLTMKAASGSTGDQKVQISYYGPKTPPVKALLYLTAVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMGPDFGYVTRGPQTGGISGLDSFGNLEVSPPVTVRGKEYPLGRILFGDSCYPSNDSRQMHQALQDFLSAQQVQAPVKLYSDWLSVGHVDEFLSFVPAPDRKGFRLLLASPRSCYKLFQEQQNEGHGEALLFEGIKKKKQQKIKNILSNKTLREHNSFVERCIDWNRELLKRELGLAESDIIDIPQLFKLKEFSKAEAFFPNMVNMLVLGKHLGIPKPFGPVINGRCCLEEKVCSLLEPLGLQCTFINDFFTYHIRHGEVHCGTNVRRKPFSFKWWNMVP

SEQ ID NO: 4 - protein, 10 aa, Homo sapiens
  REGION 1..10: note = misc_feature - HCDR1 mAb13
DFNIKDHFIH

SEQ ID NO: 5 - protein, 17 aa, Homo sapiens
  REGION 1..17: note = misc_feature - HCDR2 mAb13
RIDPEDDETKYAPKFQD

SEQ ID NO: 6 - protein, 10 aa, Homo sapiens
  REGION 1..10: note = misc_feature - HCDR3 mAb13
YGNYEGAMDY

SEQ ID NO: 7 - protein, 15 aa, Homo sapiens
  REGION 1..15: note = misc_feature - LCDR1 mAb13
RASENVNNYGIGFMN

SEQ ID NO: 8 - protein, 7 aa, Homo sapiens
  REGION 1..7: note = misc_feature - LCDR2 mAb13
AASRRGS

SEQ ID NO: 9 - protein, 9 aa, Homo sapiens
  REGION 1..9: note = misc_feature - LCDR3 mAb13
QQSKEVPYT

SEQ ID NO: 10 - protein, 119 aa, Homo sapiens
  REGION 1..119: note = misc_feature - mAb13 heavy chain variable region (VH)
EVLLQQSGAELVKPGASVRLSCTSSDFNIKDHFIHWVKQRTEQGLEWIGRIDPEDDETKYAPKFQDKVTITADTSSNTAYLQLSSLTSEDTAVFYCAGYGNYEGAMDYWGQGTSVTVSS

SEQ ID NO: 11 - DNA, 357 bp, synthetic construct
  misc_feature 1..357: note = Synthetic: mAb13 heavy chain variable region (VH) DNA sequence
  source 1..357: mol_type = other DNA
gaggttctgctgcagcagtctggggcagagcttgtgaagccgggggcctcagtcaggttgtcctgcacaagttctgacttcaatattaaagaccactttatacactgggtgaagcagaggactgaacagggcctggagtggattggaaggattgatcctgaagatgatgaaactaaatatgccccgaaattccaggacaaggtcactataacagcggacacgtcctccaacacagcctacctgcagctcagcagcctgacgtctgaggacactgccgtcttttactgtgctggttatggtaactacgaaggggctatggactactggggtcaaggaacctcagtcaccgtctcctca

SEQ ID NO: 12 - protein, 111 aa, Homo sapiens
  REGION 1..111: note = misc_feature - mAb13 light chain variable region (VL)
DIVLTQSPTSLGVSPGQRATLSCRASENVNNYGIGFMNWFQQKPGQPPKLLIYAASRRGSGVPARFSGSGSGTDFSLNIHPMEEDDTAMYFCQQSKEVPYTFGGGTNLEIR

SEQ ID NO: 13 - DNA, 333 bp, synthetic construct
  misc_feature 1..333: note = Synthetic: mAb13 light chain variable region (VL) DNA sequence
  source 1..333: mol_type = other DNA
gacattgtgctgacccaatctccaacttctttgggtgtgtctccagggcagagggccaccctctcctgcagagccagcgagaatgttaataattatggcattggttttatgaactggttccaacagaaaccaggacagccaccgaaactcctcatttatgctgcatccaggcgaggatccggggtccctgccaggtttagtggcagtgggtcagggacagacttcagcctcaacatccatcctatggaggaggatgatactgcaatgtatttctgtcaacaaagtaaggaggttccgtacacgttcggaggggggaccaacctggaaataaga

SEQ ID NO: 14 - protein, 119 aa, Homo sapiens
  REGION 1..119: note = misc_feature - hz13-1 VH
EVQLVQSGAEVKKPGASVKVSCTSSDFNIKDHFIHWVRQAPGKGLEWMGRIDPEDDETKYAPKFQDRVTMTADTSTDTAYMELSSLRSEDTAVYYCAGYGNYEGAMDYWGQGTLVTVSS

SEQ ID NO: 15 - DNA, 357 bp, synthetic construct
  misc_feature 1..357: note = Synthetic: hz13-1 VH DNA sequence
  source 1..357: mol_type = other DNA
gaggtgcagctggtgcagtccggagctgaggtgaagaagccaggagcttccgtgaaggtgagctgtacatccagcgacttcaacatcaaggatcactttatccattgggtgagacaggctccaggcaagggactggagtggatgggaaggatcgaccccgaggacgatgagacaaagtacgctcctaagttccaggatagggtgaccatgacagccgacacctctacagataccgcttatatggagctgtcttccctgagatccgaggacaccgccgtgtactattgcgctggctacggcaattatgagggcgccatggattactggggccagggcacactggtgaccgtgagctct

SEQ ID NO: 16 - protein, 111 aa, Homo sapiens
  REGION 1..111: note = misc_feature - Hz13-1 VL
DIQMTQSPSSLSASVGDRVTITCRASENVNNYGIGFMNWYQQKPGKAPKLLIYAASRRGSGVPSRFSGSGSGTDFTLTISSLQPEDFATYYCQQSKEVPYTFGQGTKVEIK

SEQ ID NO: 17 - DNA, 333 bp, synthetic construct
  misc_feature 1..333: note = Synthetic: Hz13-1 VL DNA sequence
  source 1..333: mol_type = other DNA
gacatccagatgacccagtctccatccagcctgtctgcctccgtgggcgatagagtgaccatcacatgtcgcgcttccgagaacgtgaacaattacggcatcggcttcatgaattggtatcagcagaagccaggcaaggcccccaagctgctgatctacgctgctagcaggaggggctctggagtgccctccaggttcagcggctctggctccggaaccgactttaccctgacaatctcttccctgcagcctgaggatttcgctacatactattgccagcagtccaaggaggtgccatatacctttggccagggcacaaaggtggagatcaag

SEQ ID NO: 18 - protein, 119 aa, Homo sapiens; identical to SEQ ID NO: 14
  REGION 1..119: note = misc_feature - hz13-2 VH

SEQ ID NO: 19 - DNA, 357 bp, synthetic construct; identical to SEQ ID NO: 15
  misc_feature 1..357: note = Synthetic: hz13-2 VH DNA sequence
  source 1..357: mol_type = other DNA

SEQ ID NO: 20 - protein, 111 aa, Homo sapiens
  REGION 1..111: note = misc_feature - hz13-2 VL
DIQMTQSPSSLSASVGDRVTITCRASENVNNYGIGFMNWFQQKPGKAPKLLIYAASRRGSGVPSRFSGSGSGTDFTLTISSLQPEDFATYFCQQSKEVPYTFGQGTKVEIK

SEQ ID NO: 21 - DNA, 333 bp, synthetic construct
  misc_feature 1..333: note = Synthetic: hz13-2 VL DNA sequence
  source 1..333: mol_type = other DNA
gacatccagatgacccagtctccatccagcctgtctgcctccgtgggcgatagagtgaccatcacatgtcgcgcttccgagaacgtgaacaattacggcatcggcttcatgaattggtttcagcagaagccaggcaaggcccccaagctgctgatctatgctgctagcaggaggggctctggagtgccctccaggttcagcggctctggctccggaaccgactttaccctgacaatctcttccctgcagcctgaggatttcgctacatacttttgccagcagtccaaggaggtgccatataccttcggccagggcacaaaggtggagatcaag

SEQ ID NO: 22 - protein, 119 aa, Homo sapiens; identical to SEQ ID NO: 14
  REGION 1..119: note = misc_feature - hz13-3 VH

SEQ ID NO: 23 - DNA, 357 bp, synthetic construct; identical to SEQ ID NO: 15
  misc_feature 1..357: note = Synthetic: hz13-3 VH DNA sequence
  source 1..357: mol_type = other DNA

SEQ ID NO: 24 - protein, 111 aa, Homo sapiens
  REGION 1..111: note = misc_feature - hz13-3 VL
DIVMTQSPSSLSASVGDRVTLTCRASENVNNYGIGFMNWFQQKPGKAPKLLIYAASRRGSGVPSRFSGSGSGTDFTLTISSMQPEDFATYFCQQSKEVPYTFGQGTKLEIK

SEQ ID NO: 25 - DNA, 333 bp, synthetic construct; identical to SEQ ID NO: 21
  misc_feature 1..333: note = Synthetic: hz13-3 VL DNA sequence
  source 1..333: mol_type = other DNA

SEQ ID NO: 26 - protein, 119 aa, Homo sapiens
  REGION 1..119: note = misc_feature - hz13-4 VH
EVLLVQSGAEVKKPGASVKLSCTSSDFNIKDHFIHWVRQAPGKGLEWMGRIDPEDDETKYAPKFQDRVTMTADTSTDTAYMELSSLRSEDTAVFYCAGYGNYEGAMDYWGQGTLVTVSS

SEQ ID NO: 27 - DNA, 357 bp, synthetic construct
  misc_feature 1..357: note = Synthetic: hz13-4 VH DNA sequence
  source 1..357: mol_type = other DNA
gaggtgctgctggtgcagtccggagctgaggtgaagaagccaggagcttccgtgaagctgagctgtacatccagcgacttcaacatcaaggatcactttatccattgggtgagacaggctccaggcaagggactggagtggatgggaaggatcgaccccgaggacgatgagacaaagtacgctcctaagttccaggatagggtgaccatgacagccgacacctctacagataccgcttatatggagctgtcttccctgagatccgaggacaccgccgtgttttactgcgctggctacggcaattatgagggcgccatggattattggggccagggcacactggtgaccgtgagctct

SEQ ID NO: 28 - protein, 111 aa, Homo sapiens; identical to SEQ ID NO: 16
  REGION 1..111: note = misc_feature - Hz13-4 VL

SEQ ID NO: 29 - DNA, 333 bp, synthetic construct; identical to SEQ ID NO: 17
  misc_feature 1..333: note = Synthetic: Hz13-4 VL DNA sequence
  source 1..333: mol_type = other DNA

SEQ ID NO: 30 - protein, 119 aa, Homo sapiens; identical to SEQ ID NO: 26
  REGION 1..119: note = misc_feature - hz13-5 VH

SEQ ID NO: 31 - DNA, 357 bp, synthetic construct; identical to SEQ ID NO: 27
  misc_feature 1..357: note = Synthetic: hz13-5 VH DNA sequence
  source 1..357: mol_type = other DNA

SEQ ID NO: 32 - protein, 111 aa, Homo sapiens; identical to SEQ ID NO: 20
  REGION 1..111: note = misc_feature - hz13-5 VL

SEQ ID NO: 33 - DNA, 333 bp, synthetic construct; identical to SEQ ID NO: 21
  misc_feature 1..333: note = Synthetic: hz13-5 VL DNA sequence
  source 1..333: mol_type = other DNA

SEQ ID NO: 34 - protein, 119 aa, Homo sapiens; identical to SEQ ID NO: 26
  REGION 1..119: note = misc_feature - hz13-6 VH

SEQ ID NO: 35 - DNA, 357 bp, synthetic construct; identical to SEQ ID NO: 27
  misc_feature 1..357: note = Synthetic: hz13-6 VH DNA sequence
  source 1..357: mol_type = other DNA

SEQ ID NO: 36 - protein, 111 aa, Homo sapiens; identical to SEQ ID NO: 24
  REGION 1..111: note = misc_feature - hz13-6 VL

SEQ ID NO: 37 - DNA, 333 bp, synthetic construct
  misc_feature 1..333: note = Synthetic: hz13-6 VL DNA sequence
  source 1..333: mol_type = other DNA
gacatcgtgatgacccagtctccatccagcctgtctgcctccgtgggcgatagagtgaccctgacatgtcgcgcttccgagaacgtgaacaattacggcatcggcttcatgaattggtttcagcagaagccaggcaaggcccccaagctgctgatctatgctgctagcaggaggggctctggagtgccctccaggttcagcggctctggctccggaaccgactttaccctgacaatctcttccatgcagcctgaggatttcgctacatacttttgccagcagtccaaggaggtgccatataccttcggccagggcacaaagctggagatcaag

SEQ ID NO: 38 - protein, 119 aa, Homo sapiens
  REGION 1..119: note = misc_feature - hz13-7 VH
EVLLVQSGAEVKKPGASVKLSCTSSDFNIKDHFIHWVRQAPGKGLEWIGRIDPEDDETKYAPKFQDRVTITADTSTDTAYLELSSLRSEDTAVFYCAGYGNYEGAMDYWGQGTLVTVSS

SEQ ID NO: 39 - DNA, 357 bp, synthetic construct
  misc_feature 1..357: note = Synthetic: hz137- VH DNA sequence
  source 1..357: mol_type = other DNA
gaggtgctgctggtgcagtccggagctgaggtgaagaagccaggagcttccgtgaagctgagctgtacatccagcgacttcaacatcaaggatcactttatccattgggtgagacaggctccaggcaagggactggagtggatcggaaggatcgaccccgaggacgatgagacaaagtacgctcctaagttccaggatagggtgaccatcacagccgacacctctacagataccgcttatctggagctgtcttccctgagatccgaggacaccgccgtgttttactgcgctggctacggcaattatgagggcgccatggattattggggccagggcacactggtgaccgtgagctct

SEQ ID NO: 40 - protein, 111 aa, Homo sapiens; identical to SEQ ID NO: 16
  REGION 1..111: note = misc_feature - Hz13-7 VL

SEQ ID NO: 41 - DNA, 333 bp, synthetic construct; identical to SEQ ID NO: 17
  misc_feature 1..333: note = Synthetic: Hz13-7 VL DNA sequence
  source 1..333: mol_type = other DNA

SEQ ID NO: 42 - protein, 119 aa, Homo sapiens; identical to SEQ ID NO: 38
  REGION 1..119: note = misc_feature - hz13-8 VH

SEQ ID NO: 43 - DNA, 357 bp, synthetic construct; identical to SEQ ID NO: 39
  misc_feature 1..357: note = Synthetic: hz13-8 VH DNA sequence
  source 1..357: mol_type = other DNA

SEQ ID NO: 44 - protein, 111 aa, Homo sapiens; identical to SEQ ID NO: 20
  REGION 1..111: note = misc_feature - hz13-8 VL

SEQ ID NO: 45 - DNA, 333 bp, synthetic construct; identical to SEQ ID NO: 21
  misc_feature 1..333: note = Synthetic: hz13-8 VL DNA sequence
  source 1..333: mol_type = other DNA

SEQ ID NO: 46 - protein, 119 aa, Homo sapiens; identical to SEQ ID NO: 38
  REGION 1..119: note = misc_feature - hz13-9 VH

SEQ ID NO: 47 - DNA, 357 bp, synthetic construct; identical to SEQ ID NO: 39
  misc_feature 1..357: note = Synthetic: hz13-9 VH DNA sequence
  source 1..357: mol_type = other DNA

SEQ ID NO: 48 - protein, 111 aa, Homo sapiens; identical to SEQ ID NO: 24
  REGION 1..111: note = misc_feature - hz13-9 VL

SEQ ID NO: 49 - DNA, 333 bp, synthetic construct; identical to SEQ ID NO: 37
  misc_feature 1..333: note = Synthetic: hz13-9 VL DNA sequence
  source 1..333: mol_type = other DNA

SEQ ID NO: 50 - protein, 119 aa, Homo sapiens
  REGION 1..119: note = misc_feature - hz13-10 VH
EVQLVQSGAEVKKPGATVKISCKVSDFNIKDHFIHWVQQAPGKGLEWMGRIDPEDDETKYAPKFQDRVTITADTSTDTAYMELSSLRSEDTAVYYCAGYGNYEGAMDYWGQGTTVTVSS

SEQ ID NO: 51 - DNA, 357 bp, synthetic construct
  misc_feature 1..357: note = Synthetic: hz13-10 VH DNA sequence
  source 1..357: mol_type = other DNA
gaggtgcagctggtgcagagcggagctgaggtgaagaagccaggagctaccgtgaagatctcctgtaaggtgagcgacttcaacatcaaggatcactttatccattgggtgcagcaggctcctggcaagggcctggagtggatgggcagaatcgaccccgaggacgatgagaccaagtacgctcctaagttccaggatcgcgtgaccatcacagccgacacctctacagataccgcttatatggagctgtccagcctgagatccgaggacacagccgtgtactattgcgctggctacggcaattatgagggcgccatggattactggggccagggcaccacagtgacagtgtcttcc

SEQ ID NO: 52 - protein, 111 aa, Homo sapiens
  REGION 1..111: note = misc_feature - hz13-10 VL
DVVMTQSPLSLPVTLGQPASISCRASENVNNYGIGFMNWFQQRPGQSPRLLIYAASRRGSGVPDRFSGSGSGTDFTLKISRVEEEDVGVYYCQQSKEVPYTFGGGTKVEIK

SEQ ID NO: 53 - DNA, 333 bp, synthetic construct
  misc_feature 1..333: note = Synthetic: hz13-10 VL DNA sequence
  source 1..333: mol_type = other DNA
gatgttgtgatgactcagtctccactctccctgcccgtcacccttggacagccggcctccatctcctgccgcgcttccgagaatgtgaacaattacggcatcggcttcatgaactggtttcagcagaggccaggccaatctccaaggctgctaatttatgctgcttctaggaggggctccggggtcccagacagattcagcggcagtgggtcaggcactgatttcacactgaaaatcagcagggtggaggaagaggatgttggggtttattactgccagcagtccaaggaggtgccctatacctttggcggcggcacaaaggtggagatcaag

SEQ ID NO: 54 - protein, 119 aa, Homo sapiens
  REGION 1..119: note = misc_feature - hz13-11 VH
EVQLVQSGAEVKKPGATVKISCTSSDFNIKDHFIHWVQQAPGKGLEWMGRIDPEDDETKYAPKFQDRVTITADTSTDTAYMELSSLRSEDTAVYYCAGYGNYEGAMDYWGQGTTVTVSS

SEQ ID NO: 55 - DNA, 357 bp, synthetic construct
  misc_feature 1..357: note = Synthetic: hz13-11 VH DNA sequence
  source 1..357: mol_type = other DNA
gaggtgcagctggtgcagagcggagctgaggtgaagaagccaggagctaccgtgaagatctcctgtaccagcagcgacttcaacatcaaggatcactttatccattgggtgcagcaggctcctggcaagggcctggagtggatgggcagaatcgaccccgaggacgatgagaccaagtacgctcctaagttccaggatcgcgtgaccatcacagccgacacctctacagataccgcttatatggagctgtccagcctgagatccgaggacacagccgtgtactattgcgctggctacggcaattatgagggcgccatggattactggggccagggcaccacagtgacagtgtcttcc

SEQ ID NO: 56 - protein, 111 aa, Homo sapiens
  REGION 1..111: note = misc_feature - hz13-11 VL
DVVMTQSPLSLPVTLGQPASISCRASENVNNYGIGFMNWFQQRPGQSPRRLIYAASRRGSGVPDRFSGSGSGTDFTLKISRVEAEDVGVYYCQQSKEVPYTFGGGTKVEIK

SEQ ID NO: 57 - DNA, 333 bp, synthetic construct
  misc_feature 1..333: note = Synthetic: hz13-11 VL DNA sequence
  source 1..333: mol_type = other DNA
gatgttgtgatgactcagtctccactctccctgcccgtcacccttggacagccggcctccatctcctgccgcgcttccgagaatgtgaacaattacggcatcggcttcatgaactggtttcagcagaggccaggccaatctccaaggcgcctaatttatgctgcttctaggaggggctccggggtcccagacagattcagcggcagtgggtcaggcactgatttcacactgaaaatcagcagggtggaggctgaggatgttggggtttattactgccagcagtccaaggaggtgccctatacctttggcggcggcacaaaggtggagatcaag

SEQ ID NO: 58 - protein, 119 aa, Homo sapiens; identical to SEQ ID NO: 54
  REGION 1..119: note = misc_feature - hz13-12 VH

SEQ ID NO: 59 - DNA, 357 bp, synthetic construct; identical to SEQ ID NO: 55
  misc_feature 1..357: note = Synthetic: hz13-12 VH DNA sequence
  source 1..357: mol_type = other DNA

SEQ ID NO: 60 - protein, 111 aa, Homo sapiens; identical to SEQ ID NO: 52
  REGION 1..111: note = misc_feature - hz13-12 VL

SEQ ID NO: 61 - DNA, 333 bp, synthetic construct; identical to SEQ ID NO: 53
  misc_feature 1..333: note = Synthetic: hz13-12 VL DNA sequence
  source 1..333: mol_type = other DNA

SEQ ID NO: 62 - protein, 10 aa, Homo sapiens
  REGION 1..10: note = misc_feature - HCDR1 hz13-5 D31E and hz13-12 D31E
DFNIKEHFIH

SEQ ID NO: 63 - protein, 17 aa, Homo sapiens
  REGION 1..17: note = misc_feature - HCDR2 hz13-5 D31E and hz13-12 D31E
RIDPEDDETKYAPKFQD

SEQ ID NO: 64 - protein, 10 aa, Homo sapiens
  REGION 1..10: note = misc_feature - HCDR3 hz13-5 D31E and hz13-12 D31E
YGNYEGAMDY

SEQ ID NO: 65 - protein, 15 aa, Homo sapiens
  REGION 1..15: note = misc_feature - LCDR1 hz13-5 D31E and hz13-12 D31E
RASENVNNYGIGFMN

SEQ ID NO: 66 - protein, 7 aa, Homo sapiens
  REGION 1..7: note = misc_feature - LCDR2 hz13-5 D31E and hz13-12 D31E
AASRRGS

SEQ ID NO: 67 - protein, 9 aa, Homo sapiens
  REGION 1..9: note = misc_feature - LCDR3 hz13-5 D31E and hz13-12 D31E
QQSKEVPYT

SEQ ID NO: 68 - protein, 119 aa, Homo sapiens
  REGION 1..119: note = misc_feature - hz13-5 D31E VH
EVLLVQSGAEVKKPGASVKLSCTSSDFNIKEHFIHWVRQAPGKGLEWMGRIDPEDDETKYAPKFQDRVTMTADTSTDTAYMELSSLRSEDTAVFYCAGYGNYEGAMDYWGQGTLVTVSS

SEQ ID NO: 69 - DNA, 357 bp, synthetic construct
  misc_feature 1..357: note = Synthetic: hz13-5 D31E VH DNA sequence
  source 1..357: mol_type = other DNA
gaagtgctactagtgcaaagtggtgcagaagtcaagaagcctggagcttccgtgaagctgtcctgcaccagctccgacttcaacatcaaggaacacttcatccactgggtgcggcaggcccccggcaagggcctggaatggatgggcagaatcgatcctgaggacgacgagaccaagtacgcccctaaatttcaggacagagtgaccatgacagctgataccagcaccgacaccgcctacatggaactgtcttctctgagatccgaggatacagccgtgttctactgtgctggatatggcaactacgagggcgccatggactactggggccagggcaccctggtgaccgtgtcctct

SEQ ID NO: 70 - protein, 111 aa, Homo sapiens; identical to SEQ ID NO: 20
  REGION 1..111: note = misc_feature - hz13-5 D31E VL

SEQ ID NO: 71 - DNA, 333 bp, synthetic construct
  misc_feature 1..333: note = Synthetic: hz13-5 D31E VL DNA sequence
  source 1..333: mol_type = other DNA
gatatccaaatgactcaaagtccaagtagtctgtctgcttccgtgggcgacagagtgaccatcacctgtagagcctccgagaatgtgaacaactacggcatcggcttcatgaactggttccagcagaagcctggcaaggcccctaagctgctgatctacgccgcttctcggagaggctccggcgtgcccagccggttctccggatctggctctggaaccgacttcaccctgacaatctcctccctgcagcctgaggattttgccacctacttctgccagcaaagcaaagaggttccatacaccttcggccagggcaccaaggtggaaatcaag

SEQ ID NO: 72 - protein, 10 aa, Homo sapiens
  REGION 1..10: note = misc_feature - HCDR1 mAb20
GYTFTNYGIS

SEQ ID NO: 73 - protein, 17 aa, Homo sapiens
  REGION 1..17: note = misc_feature - HCDR2 mAb20
EIYPRSGNTYHNEKFKD

SEQ ID NO: 74 - protein, 12 aa, Homo sapiens
  REGION 1..12: note = misc_feature - HCDR3 mAb20
SMITTRYWYFDV

SEQ ID NO: 75 - protein, 15 aa, Homo sapiens
  REGION 1..15: note = misc_feature - LCDR1 mAb20
RASESVDTYGNSFMH

SEQ ID NO: 76 - protein, 7 aa, Homo sapiens
  REGION 1..7: note = misc_feature - LCDR2 mAb20
LASNLES

SEQ ID NO: 77 - protein, 9 aa, Homo sapiens
  REGION 1..9: note = misc_feature - LCDR3 mAb20
QQNNEDPLT

SEQ ID NO: 78 - protein, 121 aa, Homo sapiens
  REGION 1..121: note = misc_feature - mAb20 VH
QVQLQQSGAELARPGASVKLSCKATGYTFTNYGISWVKQRTGQGLEWIGEIYPRSGNTYHNEKFKDKATLTADKSSSTAYMEFRSLTSEDSAVYFCARSMITTRYWYFDVWGKGTTVTVSS

SEQ ID NO: 79 - DNA, 363 bp, synthetic construct
  misc_feature 1..363: note = Synthetic: mAb20 VH DNA sequence
  source 1..363: mol_type = other DNA
caggttcagctgcagcagtctggagctgagctggcgaggcctggggcttcagtgaagctgtcctgcaaggctactggctacaccttcacaaactatggaataagctgggtgaagcagagaactggacagggccttgagtggattggagagatttatcctagaagtggtaatacttaccacaatgagaagttcaaggacaaggccacactgactgcagacaaatcctccagcacagcgtacatggaattccgcagcctgacatctgaggactctgcggtctatttctgtgcaagatcgatgattacgacgaggtactggtacttcgatgtctggggcaaagggaccacggtcaccgtctcctca

SEQ ID NO: 80 - protein, 111 aa, Homo sapiens
  REGION 1..111: note = misc_feature - mAb20 VL
NIVLTQSPASLAVSLGQRATLSCRASESVDTYGNSFMHWYQQKPGQPPKLLIYLASNLESGVPARFSGSGSRTDFTLTIDPVEADDAATYYCQQNNEDPLTFGAGTKLELK

SEQ ID NO: 81 - DNA, 333 bp, synthetic construct
  misc_feature 1..333: note = Synthetic: mAb20 VL DNA sequence
  source 1..333: mol_type = other DNA
aacattgtgctgacccaatctccagcttctttggctgtgtctctagggcagagggccaccttatcctgcagagccagtgaaagtgttgatacttatggcaatagttttatgcactggtaccagcagaaaccaggtcagccacccaaactcctcatctatcttgcatccaacctagaatctggggtccctgccaggttcagtggcagtgggtctaggacagacttcaccctcaccattgatcctgtggaggctgatgatgctgcaacctattactgtcagcaaaataatgaggatccgctcacgttcggtgctgggaccaagctggagctgaaa

SEQ ID NO: 82 - protein, 121 aa, Homo sapiens
  REGION 1..121: note = misc_feature - hz20-1 VH
QVQLVQSGAEVKKPGASVKVSCKASGYTFTNYGISWVRQAPGQGLEWMGEIYPRSGNTYHNEKFKDRVTMTRDTSTSTVYMELSSLRSEDTAVYYCARSMITTRYWYFDVWGKGTTVTVSS

SEQ ID NO: 83 - DNA, 363 bp, synthetic construct
  misc_feature 1..363: note = Synthetic: hz20-1 VH DNA sequence
  source 1..363: mol_type = other DNA
caggtgcagctggtgcagagcggcgccgaggtgaagaagcccggcgccagcgtgaaggtgagctgcaaggccagcggctacaccttcaccaactacggcatcagctgggtgaggcaggcccccggccagggcctggagtggatgggcgagatctaccccaggagcggcaacacctaccacaacgagaagttcaaggacagggtgaccatgaccagggacaccagcaccagcaccgtgtacatggagctgagcagcctgaggagcgaggacaccgccgtgtactactgcgccaggagcatgatcaccaccaggtactggtacttcgacgtgtggggcaagggcaccaccgtgaccgtgagcagc

SEQ ID NO: 84 - protein, 111 aa, Homo sapiens
  REGION 1..111: note = misc_feature - hz20-1 VL
DIVMTQSPDSLAVSLGERATINCRASESVDTYGNSFMHWYQQKPGQPPKLLIYLASNLESGVPDRFSGSGSGTDFTLTISSLQAEDVAVYYCQQNNEDPLTFGAGTKLELK

SEQ ID NO: 85 - DNA, 333 bp, synthetic construct
  misc_feature 1..333: note = Synthetic: hz20-1 VL DNA sequence
  source 1..333: mol_type = other DNA
gacatcgtgatgacccagtctcccgattccctggccgtgagcctgggagagagggctaccatcaactgtcgggcttccgagagcgtggacacatacggcaattctttcatgcactggtatcagcagaagcccggccagccccctaagctgctgatctacctggctagcaacctggagtctggcgtgcctgacagattctctggctccggcagcggcaccgacttcaccctgacaatctccagcctgcaggccgaggacgtggccgtgtactattgccagcagaacaatgaggaccccctgaccttcggcgctggcacaaagctggagctgaag

SEQ ID NO: 86 - protein, 121 aa, Homo sapiens
  REGION 1..121: note = misc_feature - hz20-2 VH
QVQLVQSGAEVKKPGASVKVSCKASGYTFTNYGISWVRQAPGQGLEWMGEIYPRSGNTYHNEKFKDRVTMTTDTSTSTAYMELRSLRSDDTAVYYCARSMITTRYWYFDVWGQGTLVTVSS

SEQ ID NO: 87 - DNA, 363 bp, synthetic construct
  misc_feature 1..363: note = Synthetic: hz20-2 VH DNA sequence
  source 1..363: mol_type = other DNA
caggtgcagctggtgcagagcggcgccgaggtgaagaagcccggcgccagcgtgaaggtgagctgcaaggccagcggctacaccttcaccaactacggcatcagctgggtgaggcaggcccccggccagggcctggagtggatgggcgagatctaccccaggagcggcaacacctaccacaacgagaagttcaaggacagggtgaccatgaccaccgacaccagcaccagcaccgcctacatggagctgaggagcctgaggagcgacgacaccgccgtgtactactgcgccaggagcatgatcaccaccaggtactggtacttcgacgtgtggggccagggcaccctggtgaccgtgagcagc

SEQ ID NO: 88 - protein, 111 aa, Homo sapiens
  REGION 1..111: note = misc_feature - hz20-2 VL
DIQMTQSPSSLSASVGDRVTITCRASESVDTYGNSFMHWYQQKPGKAPKLLIYLASNLESGVPSRFSGSGSGTDFTLTISSLQPEDFATYYCQQNNEDPLTFGQGTKVEIK

SEQ ID NO: 89 - DNA, 333 bp, synthetic construct
  misc_feature 1..333: note = Synthetic: hz20-2 VLDNA sequence
  source 1..333: mol_type = other DNA
gatatccagatgacccagtccccatccagcctgtctgcctccgtgggcgatagggtgaccatcacatgtcgggctagcgagtctgtggacacctacggcaacagcttcatgcactggtatcagcagaagccaggcaaggcccccaagctgctgatctacctggcttccaatctggagagcggcgtgccctctagattctccggcagcggctctggcaccgactttaccctgacaatctcttccctgcagcctgaggatttcgccacatactattgccagcagaacaatgaggacccactgacctttggccagggcacaaaggtggagatcaag

SEQ ID NO: 90 - protein, 121 aa, Homo sapiens
  REGION 1..121: note = misc_feature - hz20-3 VH
EVQLVQSGAEVKKPGASVKVSCKASGYTFTNYGISWVRQAPGQGLEWMGEIYPRSGNTYHNEKFKDRVTMTADKSTSTAYMELRSLRSDDTAVYFCARSMITTRYWYFDVWGQGTLVTVSS

SEQ ID NO: 91 - DNA, 363 bp, synthetic construct
  misc_feature 1..363: note = Synthetic: hz20-3 VH DNA sequence
  source 1..363: mol_type = other DNA
gaggtgcagctggtgcagagcggcgccgaggtgaagaagcccggcgccagcgtgaaggtgagctgcaaggccagcggctacaccttcaccaactacggcatcagctgggtgaggcaggcccccggccagggcctggagtggatgggcgagatctaccccaggagcggcaacacctaccacaacgagaagttcaaggacagggtgaccatgaccgccgacaagagcaccagcaccgcctacatggagctgaggagcctgaggagcgacgacaccgccgtgtacttctgcgccaggagcatgatcaccaccaggtactggtacttcgacgtgtggggccagggcaccctggtgaccgtgagcagc

SEQ ID NO: 92 - protein, 111 aa, Homo sapiens; identical to SEQ ID NO: 88
  REGION 1..111: note = misc_feature - hz20-3 VL

SEQ ID NO: 93 - DNA, 333 bp, synthetic construct; identical to SEQ ID NO: 89
  misc_feature 1..333: note = Synthetic: hz20-3 VLDNA sequence
  source 1..333: mol_type = other DNA

SEQ ID NO: 94 - protein, 121 aa, Homo sapiens
  REGION 1..121: note = misc_feature - hz20-4 VH
QVQLVQSGAEVKKPGASVKVSCKASGYTFTNYGISWVRQAPGQGLEWMGEIYPRSGNTYHNEKFKDRVTMTADTSTSTVYMELSSLRSEDTAVYYCARSMITTRYWYFDVWGKGTTVTVSS

SEQ ID NO: 95 - DNA, 363 bp, synthetic construct
  misc_feature 1..363: note = Synthetic: hz20-4 VH DNA sequence
  source 1..363: mol_type = other DNA
caggtgcagctggtgcagagcggcgccgaggtgaagaagcccggcgccagcgtgaaggtgagctgcaaggccagcggctacaccttcaccaactacggcatcagctgggtgaggcaggcccccggccagggcctggagtggatgggcgagatctaccccaggagcggcaacacctaccacaacgagaagttcaaggacagggtgaccatgaccgccgacaccagcaccagcaccgtgtacatggagctgagcagcctgaggagcgaggacaccgccgtgtactactgcgccaggagcatgatcaccaccaggtactggtacttcgacgtgtggggcaagggcaccaccgtgaccgtgagcagc

SEQ ID NO: 96 - protein, 111 aa, Homo sapiens; identical to SEQ ID NO: 84
  REGION 1..111: note = misc_feature - hz20-4 VL

SEQ ID NO: 97 - DNA, 333 bp, synthetic construct; identical to SEQ ID NO: 85
  misc_feature 1..333: note = Synthetic: hz20-4 VL DNA sequence
  source 1..333: mol_type = other DNA

SEQ ID NO: 98 - protein, 121 aa, Homo sapiens
  REGION 1..121: note = misc_feature - hz20-5 VH
QVQLVQSGAEVKKPGASVKVSCKASGYTFTNYGISWVRQRPGQGLEWIGEIYPRSGNTYHNEKFKDRVTMTADTSTSTVYMELSSLRSEDTAVYYCARSMITTRYWYFDVWGKGTTVTVSS

SEQ ID NO: 99 - DNA, 363 bp, synthetic construct
  misc_feature 1..363: note = Synthetic: hz20-5 VH DNA sequence
  source 1..363: mol_type = other DNA
caggtgcagctggtgcagagcggcgccgaggtgaagaagcccggcgccagcgtgaaggtgagctgcaaggccagcggctacaccttcaccaactacggcatcagctgggtgaggcagaggcccggccagggcctggagtggatcggcgagatctaccccaggagcggcaacacctaccacaacgagaagttcaaggacagggtgaccatgaccgccgacaccagcaccagcaccgtgtacatggagctgagcagcctgaggagcgaggacaccgccgtgtactactgcgccaggagcatgatcaccaccaggtactggtacttcgacgtgtggggcaagggcaccaccgtgaccgtgagcagc

SEQ ID NO: 100 - protein, 111 aa, Homo sapiens; identical to SEQ ID NO: 84
  REGION 1..111: note = misc_feature - hz20-5 VL

SEQ ID NO: 101 - DNA, 333 bp, synthetic construct; identical to SEQ ID NO: 85
  misc_feature 1..333: note = Synthetic: hz20-5 VL DNA sequence
  source 1..333: mol_type = other DNA

SEQ ID NO: 102 - protein, 121 aa, Homo sapiens
  REGION 1..121: note = misc_feature - hz20-6 VH
QVQLVQSGAEVKKPGASVKLSCKATGYTFTNYGISWVRQAPGQGLEWIGEIYPRSGNTYHNEKFKDRVTLTADKSSSTAYMEFRSLRSDDTAVYFCARSMITTRYWYFDVWGQGTLVTVSS

SEQ ID NO: 103 - DNA, 363 bp, synthetic construct
  misc_feature 1..363: note = Synthetic: hz20-6 VH DNA sequence
  source 1..363: mol_type = other DNA
caggtgcagctggtgcagagcggcgccgaggtgaagaagcccggcgccagcgtgaagctgagctgcaaggccaccggctacaccttcaccaactacggcatcagctgggtgaggcaggcccccggccagggcctggagtggatcggcgagatctaccccaggagcggcaacacctaccacaacgagaagttcaaggacagggtgaccctgaccgccgacaagagcagcagcaccgcctacatggagttcaggagcctgaggagcgacgacaccgccgtgtacttctgcgccaggagcatgatcaccaccaggtactggtacttcgacgtgtggggccagggcaccctggtgaccgtgagcagc

SEQ ID NO: 104 - protein, 111 aa, Homo sapiens; identical to SEQ ID NO: 88
  REGION 1..111: note = misc_feature - hz20-6 VL

SEQ ID NO: 105 - DNA, 333 bp, synthetic construct; identical to SEQ ID NO: 89
  misc_feature 1..333: note = Synthetic: hz20-6 VL DNA sequence
  source 1..333: mol_type = other DNA

SEQ ID NO: 106 - protein, 121 aa, Homo sapiens
  REGION 1..121: note = misc_feature - hz20-7 VH
QVQLVQSGAEVKKPGASVKVSCKASGYTFTNYGISWVRQAPGQGLEWMGEIYPRSGNTYHNEKFKDRVTMTTDTSTSTAYMELRSLRSDDTAVYYCARSMITTRYWYFDVWGKGTTVTVSS

SEQ ID NO: 107 - DNA, 363 bp, synthetic construct
  misc_feature 1..363: note = Synthetic: hz20-7 VH DNA sequence
  source 1..363: mol_type = other DNA
caggtgcagctggtgcagtctggcgccgaggtgaagaagccaggcgcttccgtgaaggtgagctgtaaggcctctggctacaccttcacaaactatggaatctcctgggtgaggcaggctccaggacagggactggagtggatgggcgagatctaccctagaagcggcaacacatatcacaatgagaagtttaaggaccgcgtgaccatgaccacagatacctctacatccaccgcctacatggagctgaggagcctgcggtctgacgatacagccgtgtactattgcgctagatccatgatcaccacacgctactggtatttcgacgtgtggggcaagggcaccacagtgaccgtgtccagc

SEQ ID NO: 108 - protein, 111 aa, Homo sapiens
  REGION 1..111: note = misc_feature - hz20-7 VL
DIVMTQSPDSLAVSLGERATINCRASESVDTYGNSFMHWYQQKPGQPPKLLIYLASNLESGVPDRFSGSGSGTDFTLTISSLQAEDVAVYYCQQNNEDPLTFGQGTKLEIK

SEQ ID NO: 109 - DNA, 333 bp, synthetic construct
  misc_feature 1..333: note = Synthetic: hz20-7 VL DNA sequence
  source 1..333: mol_type = other DNA
gacatcgtgatgacccagtctcccgattccctggccgtgagcctgggagagagggctaccatcaactgtcgggcttccgagagcgtggacacatacggcaattctttcatgcactggtatcagcagaagcccggccagccccctaagctgctgatctacctggctagcaacctggagtctggcgtgcctgacagattctctggctccggcagcggaaccgacttcaccctgacaatctccagcctgcaggccgaggacgtggccgtgtactattgccagcagaacaatgaggaccccctgaccttcggccagggcacaaagctggagatcaag

SEQ ID NO: 110 - protein, 121 aa, Homo sapiens; identical to SEQ ID NO: 106
  REGION 1..121: note = misc_feature - hz20-8 VH

SEQ ID NO: 111 - DNA, 363 bp, synthetic construct; identical to SEQ ID NO: 107
  misc_feature 1..363: note = Synthetic: hz20-8 VH DNA sequence
  source 1..363: mol_type = other DNA

SEQ ID NO: 112 - protein, 111 aa, Homo sapiens
  REGION 1..111: note = misc_feature - hz20-8 VL
DIVMTQSPDSLAVSLGERATINCRASESVDTYGNSFMHWYQQKPGQPPKLLIYLASNLESGVPDRFSGSGSGTDFTLTIDSLQAEDVAVYYCQQNNEDPLTFGQGTKLEIK

SEQ ID NO: 113 - DNA, 333 bp, synthetic construct
  misc_feature 1..333: note = Synthetic: hz20-8 VL DNA sequence
  source 1..333: mol_type = other DNA
gatatcgtgatgacccagtcccccgacagcctggccgtgtctctgggagagagggccaccatcaactgtcgggctagcgagtctgtggatacatacggcaattccttcatgcactggtatcagcagaagcccggccagccccctaagctgctgatctacctggcttctaacctggagtccggcgtgcctgacagattctccggcagcggctctggcaccgactttaccctgacaatcgattccctgcaggccgaggacgtggccgtgtactattgccagcagaacaatgaggaccccctgaccttcggccagggcacaaagctggagatcaag

SEQ ID NO: 114 - protein, 121 aa, Homo sapiens
  REGION 1..121: note = misc_feature - hz20-9 VH
QVQLVQSGAEVKKPGASVKVSCKASGYTFTNYGISWVRQAPGQGLEWMGEIYPRSGNTYHNEKFKDRVTMTTDKSTSTAYMELRSLRSDDTAVYYCARSMITTRYWYFDVWGKGTTVTVSS

SEQ ID NO: 115 - DNA, 363 bp, synthetic construct
  misc_feature 1..363: note = Synthetic: hz20-9 VH DNA sequence
  source 1..363: mol_type = other DNA
caggtgcagctggtgcagtctggcgccgaggtgaagaagccaggcgcttccgtgaaggtgagctgtaaggcctctggctacaccttcacaaactatggaatctcctgggtgaggcaggctccaggacagggactggagtggatgggcgagatctaccctagaagcggcaacacctatcacaatgagaagtttaaggaccgcgtgacaatgaccaccgataagtctacctccacagcctacatggagctgaggagcctgcggtctgacgataccgccgtgtactattgcgctagatccatgatcaccacacgctactggtatttcgacgtgtggggcaagggcaccacagtgacagtgtccagc

SEQ ID NO: 116 - protein, 111 aa, Homo sapiens; identical to SEQ ID NO: 108
  REGION 1..111: note = misc_feature - hz20-9 VL

SEQ ID NO: 117 - DNA, 333 bp, synthetic construct; identical to SEQ ID NO: 109
  misc_feature 1..333: note = Synthetic: hz20-9 VL DNA sequence
  source 1..333: mol_type = other DNA

SEQ ID NO: 118 - protein, 121 aa, Homo sapiens; identical to SEQ ID NO: 114
  REGION 1..121: note = misc_feature - hz20-10 VH

SEQ ID NO: 119 - DNA, 363 bp, synthetic construct; identical to SEQ ID NO: 115
  misc_feature 1..363: note = Synthetic: hz20-10 VH DNA sequence
  source 1..363: mol_type = other DNA

SEQ ID NO: 120 - protein, 111 aa, Homo sapiens; identical to SEQ ID NO: 112
  REGION 1..111: note = misc_feature - hz20-10 VL

SEQ ID NO: 121 - DNA, 333 bp, synthetic construct; identical to SEQ ID NO: 113
  misc_feature 1..333: note = Synthetic: hz20-10 VL DNA sequence
  source 1..333: mol_type = other DNA

SEQ ID NO: 122 - protein, 121 aa, Homo sapiens
  REGION 1..121: note = misc_feature - hz20-11 VH
QVQLVQSGAEVAKPGASVKVSCKASGYTFTNYGISWVRQRPGQGLEWMGEIYPRSGNTYHNEKFKDRVTMTTDTSTSTAYMELRSLRSEDTAVYYCARSMITTRYWYFDVWGKGTTVTVSS

SEQ ID NO: 123 - DNA, 363 bp, synthetic construct
  misc_feature 1..363: note = Synthetic: hz20-11 VH DNA sequence
  source 1..363: mol_type = other DNA
caggtgcagctggtgcagtctggagctgaggtggctaagccaggagcttccgtgaaggtgagctgtaaggcttctggctacaccttcacaaactatggcatctcctgggtgaggcagaggccaggacagggactggagtggatgggcgagatctaccctaggagcggcaacacatatcacaatgagaagtttaaggacagggtgaccatgaccacagatacctctacatccaccgcctacatggagctgagaagcctgcgctctgaggacacagccgtgtactattgcgctaggtccatgatcaccacacggtactggtatttcgacgtgtggggcaagggcaccacagtgaccgtgtccagc

SEQ ID NO: 124 - protein, 111 aa, Homo sapiens; identical to SEQ ID NO: 108
  REGION 1..111: note = misc_feature - hz20-11 VL

SEQ ID NO: 125 - DNA, 333 bp, synthetic construct; identical to SEQ ID NO: 109
  misc_feature 1..333: note = Synthetic: hz20-11 VLDNA sequence
  source 1..333: mol_type = other DNA

SEQ ID NO: 126 - protein, 121 aa, Homo sapiens; identical to SEQ ID NO: 122
  REGION 1..121: note = misc_feature - hz20-12 VH

SEQ ID NO: 127 - DNA, 363 bp, synthetic construct; identical to SEQ ID NO: 123
  misc_feature 1..363: note = Synthetic: hz20-12 VH DNA sequence
  source 1..363: mol_type = other DNA

SEQ ID NO: 128 - protein, 111 aa, Homo sapiens; identical to SEQ ID NO: 112
  REGION 1..111: note = misc_feature - hz20-12 VL

SEQ ID NO: 129 - DNA, 333 bp, synthetic construct; identical to SEQ ID NO: 113
  misc_feature 1..333: note = Synthetic: hz20-12 VL DNA sequence
  source 1..333: mol_type = other DNA

SEQ ID NO: 130 - protein, 121 aa, Homo sapiens
  REGION 1..121: note = misc_feature - hz20-13 VH
QVQLVQSGAEVKKPGASVKVSCKASGYTFTNYGISWVRQRPGQGLEWMGEIYPRSGNTYHNEKFKDRVTMTTDKSTSTAYMELRSLRSEDTAVYYCARSMITTRYWYFDVWGKGTTVTVSS

SEQ ID NO: 131 - DNA, 363 bp, synthetic construct
  misc_feature 1..363: note = Synthetic: hz20-13 VH DNA sequence
  source 1..363: mol_type = other DNA
caggtgcagctggtgcagtctggcgccgaggtgaagaagccaggcgcttccgtgaaggtgagctgtaaggcctctggctacaccttcacaaactatggcatctcctgggtgaggcagaggccaggacagggactggagtggatgggcgagatctaccctaggagcggcaacacctatcacaatgagaagtttaaggacagggtgacaatgaccaccgataagtctacctccacagcttacatggagctgagaagcctgcgctctgaggacaccgccgtgtactattgcgctaggtccatgatcaccacacggtactggtatttcgacgtgtggggcaagggcaccacagtgacagtgtccagc

SEQ ID NO: 132 - protein, 111 aa, Homo sapiens; identical to SEQ ID NO: 108
  REGION 1..111: note = misc_feature - hz20-13 VL

SEQ ID NO: 133 - DNA, 333 bp, synthetic construct; identical to SEQ ID NO: 109
  misc_feature 1..333: note = Synthetic: hz20-13 VL DNA sequence
  source 1..333: mol_type = other DNA

SEQ ID NO: 134 - protein, 121 aa, Homo sapiens; identical to SEQ ID NO: 130
  REGION 1..121: note = misc_feature - hz20-14 VH

SEQ ID NO: 135 - DNA, 363 bp, synthetic construct; identical to SEQ ID NO: 131
  misc_feature 1..363: note = Synthetic: hz20-14 VH DNA sequence
  source 1..363: mol_type = other DNA

SEQ ID NO: 136 - protein, 111 aa, Homo sapiens; identical to SEQ ID NO: 112
  REGION 1..111: note = misc_feature - hz20-14 VL

SEQ ID NO: 137 - DNA, 333 bp, synthetic construct; identical to SEQ ID NO: 113
  misc_feature 1..333: note = Synthetic: hz20-14 VL DNA sequence
  source 1..333: mol_type = other DNA

SEQ ID NO: 138 - protein, 119 aa, Homo sapiens
  REGION 1..119: note = misc_feature - hz13-5 VH_ P52aK
EVLLVQSGAEVKKPGASVKLSCTSSDFNIKDHFIHWVRQAPGKGLEWMGRIDKEDDETKYAPKFQDRVTMTADTSTDTAYMELSSLRSEDTAVFYCAGYGNYEGAMDYWGQGTLVTVSS

SEQ ID NO: 139 - DNA, 357 bp, synthetic construct
  misc_feature 1..357: note = Synthetic: hz13-5 VH_ P52aK DNA sequence
  source 1..357: mol_type = other DNA
gaggtgctgctggtgcagtctggcgctgaagtgaagaagcccggagcttctgtgaagctgtcctgcacatcctccgacttcaacatcaaggaccacttcatccactgggtgcggcaagctcctggcaagggcctggaatggatgggccggatcgacaaagaggacgacgagaccaagtacgcccctaagttccaggacagagtgaccatgaccgccgatacctctaccgatacagcctacatggaactgtctagcctgagatccgaggataccgctgtgttttattgtgccggatacggcaactacgagggcgccatggactactggggccagggcaccctggtcaccgtgtccagc

SEQ ID NO: 140 - protein, 119 aa, Homo sapiens
  REGION 1..119: note = misc_feature - hz13-5 VH_ I34H P52aK
EVLLVQSGAEVKKPGASVKLSCTSSDFNIKDHFHHWVRQAPGKGLEWMGRIDKEDDETKYAPKFQDRVTMTADTSTDTAYMELSSLRSEDTAVFYCAGYGNYEGAMDYWGQGTLVTVSS

SEQ ID NO: 141 - DNA, 357 bp, synthetic construct
  misc_feature 1..357: note = Synthetic: hz13-5 VH_ I34H P52aK DNA sequence
  source 1..357: mol_type = other DNA
gaggtgctgctggtgcagtctggcgctgaagtgaagaagcccggagcttccgtgaagctgtcttgtacctcctccgacttcaacatcaaggatcattttcaccactgggtgcggcaagctcctggcaagggcctggaatggatgggacggatcgacaaagaggacgacgagacaaagtacgcccctaagttccaggacagagtgaccatgaccgctgatacctctaccgacaccgcctacatggaactgagctctctgagatccgaggatacagccgtgttctactgcgccggctatggcaactacgagggcgccatggactactggggccagggcaccctggtcaccgtgtccagc

SEQ ID NO: 142 - protein, 119 aa, Homo sapiens
  REGION 1..119: note = misc_feature - hz13-5 VH_ I34H H35K P52aK
EVLLVQSGAEVKKPGASVKLSCTSSDFNIKDHFHKWVRQAPGKGLEWMGRIKPEDDETKYAPKFQDRVTMTADTSTDTAYMELSSLRSEDTAVFYCAGYGNYEGAMDYWGQGTLVTVSS

SEQ ID NO: 143 - DNA, 357 bp, synthetic construct
  misc_feature 1..357: note = Synthetic: hz13-5 VH_ I34H H35K P52aK DNA sequence
  source 1..357: mol_type = other DNA
gaggtgctgctggtgcagtctggcgctgaagtgaaaaaacccggagctagcgtgaagctgtcttgtacctcctccgacttcaacatcaaggaccactttcacaagtgggtgcggcaagctcctggcaagggcctggaatggatgggccggatcaagcctgaggacgacgagacaaagtacgcccctaagttccaggacagagtgaccatgaccgctgatacctctaccgataccgcctacatggaactgtccagcctgagatccgaggatacagccgtgttctactgcgccggatatggcaactacgagggcgccatggactactggggccagggcaccctggtcaccgtgtcctct

SEQ ID NO: 144 - protein, 119 aa, Homo sapiens
  REGION 1..119: note = misc_feature - hz13-5 VH_ I34H H35K
EVLLVQSGAEVKKPGASVKLSCTSSDFNIKDHFHKWVRQAPGKGLEWMGRIDPEDDETKYAPKFQDRVTMTADTSTDTAYMELSSLRSEDTAVFYCAGYGNYEGAMDYWGQGTLVTVSS

SEQ ID NO: 145 - DNA, 357 bp, synthetic construct
  misc_feature 1..357: note = Synthetic: hz13-5 VH_ I34H H35K DNA sequence
  source 1..357: mol_type = other DNA
gaggtgctgctggtgcagtccggcgctgaagtgaaaaagcctggagcctccgtgaagctgtcctgcaccagcagcgacttcaacatcaaggaccactttcacaagtgggtgcggcaagctcctggcaagggcctggaatggatgggcagaatcgaccctgaggacgacgagaccaagtacgcccccaagttccaggacagagtgaccatgaccgctgatacctctacagataccgcctacatggaactctcttctctgagatccgaggataccgccgtgttctattgtgccggctacggcaactacgagggcgccatggactactggggccagggcaccctggtcacagtgtcctct

SEQ ID NO: 146 - protein, 119 aa, Homo sapiens
  REGION 1..119: note = misc_feature - hz13-5 VH_ I34H
EVLLVQSGAEVKKPGASVKLSCTSSDFNIKDHFHHWVRQAPGKGLEWMGRIDPEDDETKYAPKFQDRVTMTADTSTDTAYMELSSLRSEDTAVFYCAGYGNYEGAMDYWGQGTLVTVSS

SEQ ID NO: 147 - DNA, 357 bp, synthetic construct
  misc_feature 1..357: note = Synthetic: hz13-5 VH_ I34H DNA sequence
  source 1..357: mol_type = other DNA
gaagtgctgctggtgcagagcggagctgaggtgaaaaagcccggagcttctgtgaagctgtcttgtacctcctccgacttcaacatcaaggaccactttcatcactgggtgcggcaagctcctggcaagggcctggaatggatgggccggatcgatcctgaggacgacgagacaaagtacgcccctaagttccaggacagagtgaccatgaccgctgatacctctaccgacaccgcctacatggaactgtcctctctgagatccgaggatacagccgtgttctactgcgccggctatggcaactacgagggcgccatggactactggggccagggcaccctggtcaccgtgtccagc

SEQ ID NO: 148 - protein, 119 aa, Homo sapiens
  REGION 1..119: note = misc_feature - hz13-5 VH_ H35K P52aK
EVLLVQSGAEVKKPGASVKLSCTSSDFNIKDHFIKWVRQAPGKGLEWMGRIDKEDDETKYAPKFQDRVTMTADTSTDTAYMELSSLRSEDTAVFYCAGYGNYEGAMDYWGQGTLVTVSS

SEQ ID NO: 149 - DNA, 357 bp, synthetic construct
  misc_feature 1..357: note = Synthetic: hz13-5 VH_ H35K P52aK DNA sequence
  source 1..357: mol_type = other DNA
gaagtgctgctggtgcagtctggcgctgaggtgaaaaagcccggagcttccgtgaagctgtcctgcacctcttccgacttcaacatcaaggaccacttcatcaagtgggtgcggcaagctcctggcaagggcctggaatggatgggccggatcgacaaagaggacgacgagaccaagtacgcccctaagttccaggacagagtgaccatgaccgctgatacaagcaccgataccgcctacatggaactgtcttctctgagatccgaggatacagccgtgttttattgtgccggctacggcaactacgagggcgccatggactactggggccagggcaccctggtcaccgtgtccagc

SEQ ID NO: 150 - protein, 119 aa, Homo sapiens
  REGION 1..119: note = misc_feature - hz13-5 VH_ H35K
EVLLVQSGAEVKKPGASVKLSCTSSDFNIKDHFIKWVRQAPGKGLEWMGRIDPEDDETKYAPKFQDRVTMTADTSTDTAYMELSSLRSEDTAVFYCAGYGNYEGAMDYWGQGTLVTVSS

SEQ ID NO: 151 - DNA, 357 bp, synthetic construct
  misc_feature 1..357: note = Synthetic: hz13-5 VH_ H35K DNA sequence
  source 1..357: mol_type = other DNA
gaagtgctgctggtgcagtctggcgctgaggtgaaaaagcctggagcctccgtgaagctgtcttgtacctcctccgacttcaacatcaaggaccacttcatcaagtgggtgcggcaagctcctggcaagggcctggaatggatgggccggatcgaccccgaggacgacgagaccaagtacgcccctaagtttcaggacagagtgaccatgaccgctgatacatctaccgataccgcctacatggaactgagcagcctgagatccgaggatacagccgtgttctactgcgctggctatggcaactacgagggcgccatggactactggggccagggcaccctggtcaccgtgtcctct

SEQ ID NO: 152 - protein, 119 aa, Homo sapiens
  REGION 1..119: note = misc_feature - hz13-5 VH_ H32W
EVLLVQSGAEVKKPGASVKLSCTSSDFNIKDWFIHWVRQAPGKGLEWMGRIDPEDDETKYAPKFQDRVTMTADTSTDTAYMELSSLRSEDTAVFYCAGYGNYEGAMDYWGQGTLVTVSS

SEQ ID NO: 153 - DNA, 357 bp, synthetic construct
  misc_feature 1..357: note = Synthetic: hz13-5 VH_ H32W DNA sequence
  source 1..357: mol_type = other DNA
gaagtgctgctggtgcagtctggcgctgaggtgaaaaagcctggagcctccgtgaagctgtcctgcacctcctccgacttcaacatcaaggactggttcatccactgggtcagacaggctcctggcaagggcctggaatggatgggccggatcgaccccgaggacgacgagaccaagtacgcccctaagttccaggacagagtgaccatgaccgctgatacatctaccgataccgcctacatggaactgtcttctctgcggagcgaggatacagccgtgttttattgtgctggctacggcaactacgagggcgccatggactactggggccaaggaaccctcgtgaccgtgtccagc

SEQ ID NO: 154 - protein, 119 aa, Homo sapiens
  REGION 1..119: note = misc_feature - hz13-5 VH_ D31H P52aK
EVLLVQSGAEVKKPGASVKLSCTSSDFNIKHHFIHWVRQAPGKGLEWMGRIDKEDDETKYAPKFQDRVTMTADTSTDTAYMELSSLRSEDTAVFYCAGYGNYEGAMDYWGQGTLVTVSS

SEQ ID NO: 155 - DNA, 357 bp, synthetic construct
  misc_feature 1..357: note = Synthetic: hz13-5 VH_ D31H P52aK DNA sequence
  source 1..357: mol_type = other DNA
gaggtgctgctggtgcagagcggagctgaagtgaagaagcctggcgcttctgtgaagctgtcctgcacctcctccgatttcaacatcaagcaccattttatccactgggtgcggcaagctcccggcaagggcctggaatggatgggccggatcgacaaagaggacgacgagaccaagtacgcccctaagttccaggacagagtgaccatgaccgctgatacatctaccgacaccgcctacatggaactgtctagcctgagatccgaggatacagccgtgttctactgtgccggctatggaaactacgagggcgccatggactactggggccagggcaccctggtcaccgtgtcctct

SEQ ID NO: 156 - protein, 119 aa, Homo sapiens
  REGION 1..119: note = misc_feature - hz13-5 VH_ D31H I34H P52aK
EVLLVQSGAEVKKPGASVKLSCTSSDFNIKHHFHHWVRQAPGKGLEWMGRIDKEDDETKYAPKFQDRVTMTADTSTDTAYMELSSLRSEDTAVFYCAGYGNYEGAMDYWGQGTLVTVSS

SEQ ID NO: 157 - DNA, 357 bp, synthetic construct
  misc_feature 1..357: note = Synthetic: hz13-5 VH_ D31H I34H P52aK DNA sequence
  source 1..357: mol_type = other DNA
gaagtgctgctggtgcagtctggcgccgaggtgaagaagcccggagcttctgtgaagctgtcctgcacctcctccgacttcaacatcaagcaccattttcaccactgggtgcggcaagctcctggcaagggcctggaatggatgggccggatcgataaagaggacgacgagacaaagtacgcccctaagttccaggacagagtgaccatgaccgctgatacctctaccgacaccgcctacatggaactgtctagcctgagatccgaggatacagccgtgttctattgtgccggctacggcaactacgagggagccatggactactggggccagggcaccctggtcaccgtgtccagc

SEQ ID NO: 158 - protein, 119 aa, Homo sapiens
  REGION 1..119: note = misc_feature - hz13-5 VH_ D31H I34H H35K P52aK
EVLLVQSGAEVKKPGASVKLSCTSSDFNIKHHFHKWVRQAPGKGLEWMGRIDKEDDETKYAPKFQDRVTMTADTSTDTAYMELSSLRSEDTAVFYCAGYGNYEGAMDYWGQGTLVTVSS

SEQ ID NO: 159 - DNA, 357 bp, synthetic construct
  misc_feature 1..357: note = Synthetic: hz13-5 VH_ D31H I34H H35K P52aK DNA sequence
  source 1..357: mol_type = other DNA
gaggtgctgctggtgcagtccggcgctgaagtgaaaaagcctggagcttctgtgaagctgtcctgcacctcttccgacttcaacatcaagcaccatttccacaagtgggtgcggcaagcccctggcaagggcctggaatggatgggccggatcgacaaagaggacgacgagaccaagtacgcccccaagttccaggacagagtgaccatgaccgctgatacaagcaccgataccgcctacatggaactgtcttctctgagatccgaggatacagccgtgttttattgtgctggctacggcaactacgagggcgccatggactactggggccagggcaccctggtcaccgtgtccagc

SEQ ID NO: 160 - protein, 119 aa, Homo sapiens
  REGION 1..119: note = misc_feature - hz13-5 VH_ D31H I34H H35K
EVLLVQSGAEVKKPGASVKLSCTSSDFNIKHHFHKWVRQAPGKGLEWMGRIDPEDDETKYAPKFQDRVTMTADTSTDTAYMELSSLRSEDTAVFYCAGYGNYEGAMDYWGQGTLVTVSS

SEQ ID NO: 161 - DNA, 357 bp, synthetic construct
  misc_feature 1..357: note = Synthetic: hz13-5 VH_ D31H I34H H35K DNA sequence
  source 1..357: mol_type = other DNA
gaggtgctgctggtgcagtctggagctgaagtgaaaaagcctggagcttccgtgaagctgtcctgcacctctagcgacttcaacatcaagcaccattttcacaagtgggtgcggcaagctcctggcaagggcctggaatggatgggccggatcgaccccgaggacgacgagacaaagtacgcccctaagttccaggacagagtgaccatgaccgctgatacctccaccgacaccgcctacatggaactctcttctctgagatccgaggatacagccgtgttctattgtgccggctacggcaactacgagggcgccatggattactggggccagggcaccctggtcaccgtgtccagc

SEQ ID NO: 162 - protein, 119 aa, Homo sapiens
  REGION 1..119: note = misc_feature - hz13-5 VH_ D31H I34H
EVLLVQSGAEVKKPGASVKLSCTSSDFNIKHHFHHWVRQAPGKGLEWMGRIDPEDDETKYAPKFQDRVTMTADTSTDTAYMELSSLRSEDTAVFYCAGYGNYEGAMDYWGQGTLVTVSS

SEQ ID NO: 163 - DNA, 357 bp, synthetic construct
  misc_feature 1..357: note = Synthetic: hz13-5 VH_ D31H I34H DNA sequence
  source 1..357: mol_type = other DNA
gaggtgctgctggtgcagtccggcgctgaagtgaagaaacctggcgcttctgtcaagctgtcctgtacatctagcgacttcaacatcaagcaccacttccatcactgggtgcggcaagctcctggaaagggcctggaatggatgggcagaatcgaccctgaggacgacgagaccaagtacgcccccaagtttcaggacagagtgaccatgaccgccgatacctccaccgatacagcctacatggaactgtcttctctgcggtccgaggataccgctgtgttctactgcgccggctatggcaactacgagggagccatggactactggggccagggcaccctcgtgaccgtgtccagc

SEQ ID NO: 164 - protein, 119 aa, Homo sapiens
  REGION 1..119: note = misc_feature - hz13-5 VH_ D31H H35K P52aK
EVLLVQSGAEVKKPGASVKLSCTSSDFNIKHHFIKWVRQAPGKGLEWMGRIDKEDDETKYAPKFQDRVTMTADTSTDTAYMELSSLRSEDTAVFYCAGYGNYEGAMDYWGQGTLVTVSS

SEQ ID NO: 165 - DNA, 357 bp, synthetic construct
  misc_feature 1..357: note = Synthetic: hz13-5 VH_ D31H H35K P52aK DNA sequence
  source 1..357: mol_type = other DNA
gaggtgctgctggtgcagagcggagctgaagtgaaaaagcccggagcttctgtgaagctgtcttgtacctcctccgacttcaacatcaagcaccacttcatcaagtgggtgcggcaagctcctggcaagggcctggaatggatgggccggatcgacaaagaggacgacgagaccaagtacgcccctaagtttcaggacagagtgaccatgaccgctgatacatctaccgatacagcctacatggaactgtcctctctgagatccgaggataccgccgtgttctactgcgccggctatggcaactacgagggcgccatggactactggggccagggcaccctggtcaccgtgtccagc

SEQ ID NO: 166 - protein, 119 aa, Homo sapiens
  REGION 1..119: note = misc_feature - hz13-5 VH_ D31H H35K
EVLLVQSGAEVKKPGASVKLSCTSSDFNIKHHFIKWVRQAPGKGLEWMGRIDPEDDETKYAPKFQDRVTMTADTSTDTAYMELSSLRSEDTAVFYCAGYGNYEGAMDYWGQGTLVTVSS

SEQ ID NO: 167 - DNA, 357 bp, synthetic construct
  misc_feature 1..357: note = Synthetic: hz13-5 VH_ D31H H35K DNA sequence
  source 1..357: mol_type = other DNA
gaggtgctgctggtgcagtctggcgctgaagtgaaaaagcctggagcttccgtgaagctgtcctgcacctctagcgacttcaacatcaagcaccacttcatcaagtgggtgcggcaagctcctggcaagggcctggaatggatgggccggatcgatcccgaggacgacgagaccaagtacgcccctaagttccaggacagagtgaccatgaccgctgatacctccaccgacacagcctacatggaactgtcttctctgagatccgaggataccgccgtgttttattgtgccggctacggcaactacgagggagccatggactactggggccagggcaccctggtcacagtgtccagc

SEQ ID NO: 168 - protein, 119 aa, Homo sapiens
  REGION 1..119: note = misc_feature - hz13-5 VH_ D31H
EVLLVQSGAEVKKPGASVKLSCTSSDFNIKHHFIHWVRQAPGKGLEWMGRIDPEDDETKYAPKFQDRVTMTADTSTDTAYMELSSLRSEDTAVFYCAGYGNYEGAMDYWGQGTLVTVSS

SEQ ID NO: 169 - DNA, 357 bp, synthetic construct
  misc_feature 1..357: note = Synthetic: hz13-5 VH_ D31H DNA sequence
  source 1..357: mol_type = other DNA
gaggtgctgctggtgcagtctggcgctgaagtgaaaaagcctggagcctccgtgaagctgtcttgcacctcctccgacttcaacatcaagcaccattttatccactgggtgcggcaagctcctggcaagggcctggaatggatgggccggatcgaccccgaggacgacgagaccaagtacgcccctaagttccaggacagagtgaccatgaccgctgatacatctaccgataccgcctacatggaactcagctctctgagatccgaggatacagccgtgttctattgtgctggctacggcaactacgagggcgccatggactactggggccagggcaccctggtcaccgtgtccagc

SEQ ID NO: 170 - protein, 111 aa, Homo sapiens; identical to SEQ ID NO: 20
  REGION 1..111: note = misc_feature - VL sequence for hz13-5 VH_ P52aK, hz13-5 VH_ I34H P52aK, hz13-5 VH_ I34H H35K P52aK, hz13-5 VH_ I34H H35K, hz13-5 VH_ I34H, hz13-5 VH_ H35K P52aK, hz13-5 VH_ H35K, hz13-5 VH_ H32W, hz13-5 VH_ D31H P52aK, hz13-5 VH_ D31H I34H P52aK, hz13-5 VH_ D31H I34H H35K P52aK, hz13-5 VH_ D31H I34H H35K, hz13-5 VH_ D31H I34H, hz13-5 VH_ D31H H35K P52aK, hz13-5 VH_ D31H H35K, hz13-5 VH_ D31H

SEQ ID NO: 171 - DNA, 333 bp, synthetic construct; identical to SEQ ID NO: 21
  misc_feature 1..333: note = Synthetic: VL DNA sequence for hz13-5 VH_ P52aK, hz13-5 VH_ I34H P52aK, hz13-5 VH_ I34H H35K P52aK, hz13-5 VH_ I34H H35K, hz13-5 VH_ I34H, hz13-5 VH_ H35K P52aK, hz13-5 VH_ H35K, hz13-5 VH_ H32W, hz13-5 VH_ D31H P52aK, hz13-5 VH_ D31H I34H P52aK, hz13-5 VH_ D31H I34H H35K P52aK, hz13-5 VH_ D31H I34H H35K, hz13-5 VH_ D31H I34H, hz13-5 VH_ D31H H35K P52aK, hz13-5 VH_ D31H H35K, hz13-5 VH_ D31H
  source 1..333: mol_type = other DNA

SEQ ID NO: 172 - protein, 111 aa, Homo sapiens
  REGION 1..111: note = misc_feature - VL_I30H protein sequence for hz13-5 VH_ P52aK::Vk_I30H, hz13-5 VH_ I34H, P52aK::Vk_ I30H, hz13-5 VH_ I34H H35K P52aK::Vk_I30H, hz13-5 VH_ I34H H35K::VK_I30H, hz13-5 VH_ I34H::Vk_I30H, hz13-5 VH_ H35K P52aK::Vk_I30H, hz13-5 VH_ H35K::Vk_ I30H, hz13-5 VH_ D31H P52aK::Vk_ I30H, hz13-5 VH_ D31H I34H P52aK::Vk_ I30H, hz13-5 VH_ D31H I34H H35K P52aK::Vk_ I30H, hz13-5 VH_ D31H I34H H35K::Vk_ I30H, hz13-5 VH_ D31H I34H::Vk_I30H, hz13-5 VH_ D31H H35K P52aK::Vk_ I30H, hz13-5 VH_ D31H H35K::Vk_I30H, hz13-5 VH_ D31H::Vk_I30H
DIQMTQSPSSLSASVGDRVTITCRASENVNNYGHGFMNWFQQKPGKAPKLLIYAASRRGSGVPSRFSGSGSGTDFTLTISSLQPEDFATYFCQQSKEVPYTFGQGTKVEIK

SEQ ID NO: 173 - DNA, 333 bp, synthetic construct
  misc_feature 1..333: note = Synthetic: Vk_I30H DNA sequence for hz13-5 VH_ P52aK::Vk_I30H, hz13-5 VH_ I34H, P52aK::Vk_ I30H, hz13-5 VH_ I34H H35K P52aK::Vk_I30H, hz13-5 VH_ I34H H35K::VK_I30H, hz13-5 VH_ I34H::Vk_I30H, hz13-5 VH_ H35K P52aK::Vk_I30H, hz13-5 VH_ H35K::Vk_ I30H, hz13-5 VH_ D31H P52aK::Vk_ I30H, hz13-5 VH_ D31H I34H P52aK::Vk_ I30H, hz13-5 VH_ D31H I34H H35K P52aK::Vk_ I30H, hz13-5 VH_ D31H I34H H35K::Vk_ I30H, hz13-5 VH_ D31H I34H::Vk_I30H, hz13-5 VH_ D31H H35K P52aK::Vk_ I30H, hz13-5 VH_ D31H H35K::Vk_I30H, hz13-5 VH_ D31H::Vk_I30H
  source 1..333: mol_type = other DNA
gatatccagatgacccagtctccatccagcctgtctgcttccgtgggcgacagagtcacaatcacctgtagagcctccgaaaacgtgaacaactacggccatggcttcatgaactggttccagcagaagcctggcaaggcccccaaactgctgatctacgctgcttcacgccggggatctggcgtgccctcccggttctccggctccggctctggaaccgattttaccctgaccatctcctccctgcagcctgaggacttcgccacctacttctgccagcagtccaaagaggtgccttacacctttggccagggcaccaaagtggaaatcaag

SEQ ID NO: 174 - protein, 330 aa, Homo sapiens
  REGION 1..330: note = misc_feature - hIgG1 HC constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - DNA, 990 bp, synthetic construct
  misc_feature 1..990: note = Synthetic: hIgG1 HC constant region DNA
  source 1..990: mol_type = other DNA
gcgtcgaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaag

SEQ ID NO: 176 - protein, 329 aa, Homo sapiens
  REGION 1..329: note = misc_feature - hIgG1 HC constant region without C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 177 - DNA, 987 bp, synthetic construct
  misc_feature 1..987: note = Synthetic: hIgG1 HC constant region DNA without C-terminal lysine
  source 1..987: mol_type = other DNA
gcgtcgaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 178 - protein, 330 aa, Homo sapiens
  REGION 1..330: note = misc_feature - hIgG1.3f HC constant region; IgG 1.3 comprises mutations L234A, L235E, and G237A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - DNA, 990 bp, synthetic construct
  misc_feature 1..990: note = Synthetic: hIgG1.3f HC constant region DNA sequence
  source 1..990: mol_type = other DNA
gcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaag

SEQ ID NO: 180 - protein, 329 aa, Homo sapiens
  REGION 1..329: note = misc_feature - hIgG1.3f HC constant region without the C-terminal lysine; IgG 1.3 mutations L234A, L235E, and G237A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 181 - DNA, 987 bp, synthetic construct
  misc_feature 1..987: note = Synthetic: hIgG1.3f HC constant region without the C-terminal lysine; DNA sequence
  source 1..987: mol_type = other DNA
gcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 182 - protein, 330 aa, Homo sapiens
  REGION 1..330: note = misc_feature - hIgG1fa L235E, P238K (LEPK) HC constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELEGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - DNA, 990 bp, synthetic construct
  misc_feature 1..990: note = Synthetic: hIgG1fa LEPK HC constant region DNA sequence
  source 1..990: mol_type = other DNA
gcgtcgaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcgaggggggaaagtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 184 - protein, 329 aa, Homo sapiens
  REGION 1..329: note = misc_feature - hIgG1fa LEPK HC constant region without the C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELEGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 185 - DNA, 987 bp, synthetic construct
  misc_feature 1..987: note = Synthetic: hIgG1fa LEPK HC constant region without the C-terminal lysine DNA sequence
  source 1..987: mol_type = other DNA
gcgtcgaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcgaggggggaaagtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 186 - protein, 326 aa, Homo sapiens
  REGION 1..326: note = misc_feature - hIgG2.3 HC constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 187 - DNA, 978 bp, synthetic construct
  misc_feature 1..978: note = Synthetic: hIgG2.3 HC constant region DNA
  source 1..978: mol_type = other DNA
gcgtcgaccaagggcccctctgtgtttcctctggccccttgctcccggtccacctctgagtctaccgctgctctgggctgcctggtcaaggactacttccccgagcctgtgaccgtgtcctggaactctggcgctctgacctccggcgtgcacacctttccagccgtgctgcagtcctccggcctgtactctctgtcctccgtcgtgaccgtgccctcctccaacttcggcacccagacctacacctgtaacgtggaccacaagccctccaacaccaaggtggacaagaccgtggaacggaagtcctgcgtggaatgccctccttgccctgcacctcctgtggctggcaaatccgtgttcctgttccccccaaagcccaaggacaccctgatgatctcccggacccccgaagtgacctgcgtggtggtggatgtgtcccacgaggaccccgaggtgcagttcaattggtacgtggacggcgtggaagtgcacaacgccaagaccaagcccagagaggaacagttcaactccaccttccgggtggtgtccgtgctgaccgtggtgcaccaggactggctgaacggcaaagagtacaagtgcaaggtgtccaacaagggcctgcctgcccccatcgaaaagaccatctccaagacaaagggccagccccgcgagcctcaggtgtacacactgcctcccagccgggaagagatgaccaagaaccaggtgtccctgacctgtctggtcaagggcttctacccctccgatatcgccgtggaatgggagtccaacggccagcccgagaacaactacaagaccaccccccccatgctggactccgacggctcattcttcctgtactccaagctgacagtggacaagtcccggtggcagcagggcaacgtgttctcctgctctgtgatgcacgaggccctgcacaaccactacacccagaagtccctgtccctgagccccggcaaa

SEQ ID NO: 188 - protein, 325 aa, Homo sapiens
  REGION 1..325: note = misc_feature - hIgG2.3 HC constant region, without C-terminal lysine
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 189 - DNA, 975 bp, synthetic construct
  misc_feature 1..975: note = Synthetic: hIgG2.3 HC constant region, without C-terminal lysine DNA
  source 1..975: mol_type = other DNA
gcgtcgaccaagggcccctctgtgtttcctctggccccttgctcccggtccacctctgagtctaccgctgctctgggctgcctggtcaaggactacttccccgagcctgtgaccgtgtcctggaactctggcgctctgacctccggcgtgcacacctttccagccgtgctgcagtcctccggcctgtactctctgtcctccgtcgtgaccgtgccctcctccaacttcggcacccagacctacacctgtaacgtggaccacaagccctccaacaccaaggtggacaagaccgtggaacggaagtcctgcgtggaatgccctccttgccctgcacctcctgtggctggcaaatccgtgttcctgttccccccaaagcccaaggacaccctgatgatctcccggacccccgaagtgacctgcgtggtggtggatgtgtcccacgaggaccccgaggtgcagttcaattggtacgtggacggcgtggaagtgcacaacgccaagaccaagcccagagaggaacagttcaactccaccttccgggtggtgtccgtgctgaccgtggtgcaccaggactggctgaacggcaaagagtacaagtgcaaggtgtccaacaagggcctgcctgcccccatcgaaaagaccatctccaagacaaagggccagccccgcgagcctcaggtgtacacactgcctcccagccgggaagagatgaccaagaaccaggtgtccctgacctgtctggtcaagggcttctacccctccgatatcgccgtggaatgggagtccaacggccagcccgagaacaactacaagaccaccccccccatgctggactccgacggctcattcttcctgtactccaagctgacagtggacaagtcccggtggcagcagggcaacgtgttctcctgctctgtgatgcacgaggccctgcacaaccactacacccagaagtccctgtccctgagccccggc

SEQ ID NO: 190 - protein, 326 aa, Homo sapiens
  REGION 1..326: note = misc_feature - hIgG2.3 P238K HC constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGKSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 191 - DNA, 978 bp, synthetic construct; identical to SEQ ID NO: 187
  misc_feature 1..978: note = Synthetic: hIgG2.3 P238K HC constant region DNA sequence
  source 1..978: mol_type = other DNA

SEQ ID NO: 192 - protein, 325 aa, Homo sapiens
  REGION 1..325: note = misc_feature - hIgG2.3 P238K HC constant region without the C-terminal lysine
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGKSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 193 - DNA, 975 bp, synthetic construct; identical to SEQ ID NO: 189
  misc_feature 1..975: note = Synthetic: hIgG2.3 P238K HC constant region without the C-terminal lysine DNA sequence
  source 1..975: mol_type = other DNA

SEQ ID NO: 194 - protein, 107 aa, Homo sapiens [sequence omitted]

SEQ ID NO: 195 - DNA, 321 bp, synthetic construct
  misc_feature 1..321: note = Synthetic: Exemplary LC constant region DNA sequence
  source 1..321: mol_type = other DNA
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 196 - protein, 449 aa, Homo sapiens
  REGION 1..449: note = misc_feature - hz13 -5 D31E HC
EVLLVQSGAEVKKPGASVKLSCTSSDFNIKEHFIHWVRQAPGKGLEWMGRIDPEDDETKYAPKFQDRVTMTADTSTDTAYMELSSLRSEDTAVFYCAGYGNYEGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - DNA, 1347 bp, synthetic construct
  misc_feature 1..1347: note = Synthetic: hz13 -5 D31E HC DNA sequence
  source 1..1347: mol_type = other DNA
gaagtgctgctggtgcagtctggcgctgaggtgaagaagcctggagctagcgtgaagctgtcctgcacctcctccgacttcaacatcaaagagcacttcatccactgggtgcggcaggcccctggcaagggcctggaatggatgggccggatcgaccctgaggacgacgagaccaagtacgcccccaagtttcaagatagagtgaccatgaccgccgatacctctaccgacacagcctacatggaactgagctctctgagatccgaggatacagctgtgttctattgtgctggctacggcaactacgagggagccatggactactggggccagggcaccctcgtcaccgtgtcctctgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 198 - protein, 448 aa, Homo sapiens
  REGION 1..448: note = misc_feature - hz13 -5 D31E HC, without C-terminal lysine
EVLLVQSGAEVKKPGASVKLSCTSSDFNIKEHFIHWVRQAPGKGLEWMGRIDPEDDETKYAPKFQDRVTMTADTSTDTAYMELSSLRSEDTAVFYCAGYGNYEGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 199 - DNA, 1344 bp, synthetic construct
  misc_feature 1..1344: note = Synthetic: hz13 -5 D31E HC DNA sequence, without C-terminal lysine
  source 1..1344: mol_type = other DNA
gaagtgctactagtgcaaagtggtgcagaagtcaagaagcctggagcttccgtgaagctgtcctgcaccagctccgacttcaacatcaaggaacacttcatccactgggtgcggcaggcccccggcaagggcctggaatggatgggcagaatcgatcctgaggacgacgagaccaagtacgcccctaaatttcaggacagagtgaccatgacagctgataccagcaccgacaccgcctacatggaactgtcttctctgagatccgaggatacagccgtgttctactgtgctggatatggcaactacgagggcgccatggactactggggccagggcaccctggtgaccgtgtcctctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 200 - protein, 218 aa, Homo sapiens
  REGION 1..218: note = misc_feature - hz13-5 D31E LC
DIQMTQSPSSLSASVGDRVTITCRASENVNNYGIGFMNWFQQKPGKAPKLLIYAASRRGSGVPSRFSGSGSGTDFTLTISSLQPEDFATYFCQQSKEVPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - DNA, 654 bp, synthetic construct
  misc_feature 1..654: note = Synthetic: hz13-5 D31E LC DNA sequence
  source 1..654: mol_type = other DNA
gacatccagatgacccaatcgccatcgtccctgtccgcatccgtgggagacagagtgacaattacttgccgggccagcgaaaacgtgaacaactacggcattggcttcatgaactggttccagcaaaagccaggaaaggccccgaagctcctgatctacgccgcaagccggagaggttccggagtgccctcacggttctccggctccggatctggaaccgacttcacactgaccattagcagcctccagcctgaggacttcgccacctacttctgccaacaatccaaggaggtgccctacacctttggacaaggcaccaaggtcgagatcaagaggacggtggcggcaccctccgtgtttatcttcccgccgtccgacgaacagcttaagtccggcactgcttcggtcgtgtgtctgctgaacaacttctatcctcgcgaggccaaagtgcagtggaaggtcgataacgcgctgcagtccgggaattcacaggaatcagtgaccgaacaggattcgaaggactccacttactcgctgtcgagcactctcactctgagcaaggccgactacgaaaagcacaaagtgtacgcctgcgaagtgacccatcagggtttgtcctcccctgtgaccaagagcttcaacagaggggagtgc

SEQ ID NO: 202 - protein, 449 aa, Homo sapiens
  REGION 1..449: note = misc_feature - hz13-12 D31E HC
EVQLVQSGAEVKKPGATVKISCTSSDFNIKEHFIHWVQQAPGKGLEWMGRIDPEDDETKYAPKFQDRVTITADTSTDTAYMELSSLRSEDTAVYYCAGYGNYEGAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 203 - DNA, 1347 bp, synthetic construct
  misc_feature 1..1347: note = Synthetic: hz13-12 D31E HC DNA sequence
  source 1..1347: mol_type = other DNA
gaggtgcagctggtgcaatctggcgctgaagtgaagaagcctggagctaccgtcaagatctcctgcacctcctccgacttcaacatcaaagagcacttcatccactgggtgcagcaggcccctggcaagggcctggaatggatgggccggatcgatcctgaggacgacgagacaaagtacgcccccaagtttcaggacagagtgaccatcaccgctgatacatctaccgacaccgcctacatggaactgagctctctgcggtccgaggataccgccgtgtactattgtgctggctacggcaactacgagggcgccatggactactggggccagggcaccaccgtgacagtgtccagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 204 - protein, 448 aa, Homo sapiens
  REGION 1..448: note = misc_feature - hz13-12 D31E HC, without C-terminal lysine
EVQLVQSGAEVKKPGATVKISCTSSDFNIKEHFIHWVQQAPGKGLEWMGRIDPEDDETKYAPKFQDRVTITADTSTDTAYMELSSLRSEDTAVYYCAGYGNYEGAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 205 - DNA, 1344 bp, synthetic construct
  misc_feature 1..1344: note = Synthetic: hz13-12 D31E HC DNA sequence, without C-terminal lysine
  source 1..1344: mol_type = other DNA
gaggtgcaactggtgcagtcgggagccgaagtcaagaagcctggagccaccgtgaagatcagttgcaccagttccgatttcaacatcaaggagcacttcatccactgggtgcaacaggctccgggaaagggcctcgagtggatggggcggattgatcccgaggacgacgaaaccaaatacgccccgaagtttcaggaccgggtcaccattaccgccgacaccagcaccgacactgcgtacatggaactgtcgtccctgcgctccgaggataccgccgtgtactattgcgccggttacgggaactacgagggtgccatggattactggggacagggaaccaccgtgactgtgtccagcgcgtccactaagggaccttcagtgttcccgctggctcctagctcaaagtccacctcgggaggcactgcagctctgggttgcctcgtgaaggactacttccctgaacctgtgaccgtgtcctggaattccggcgctcttacctccggagtgcatactttccccgccgtgctccaaagctccggcctgtacagcctgtcctccgtcgtgactgtcccgagctcgtccctgggaacccagacttacatttgcaacgtgaaccacaagccctcaaacaccaaggtcgataagagggtggaacccaagagctgtgacaagactcacacttgcccgccatgtccagcccctgaagccgagggtgccccatctgtgtttctgttcccgcccaaaccgaaggacaccctgatgatctcaagaaccccggaggtcacctgtgtggtggtcgacgtgtcccacgaggatcccgaagtgaaattcaattggtacgtggacggggtggaggtccataacgccaagacaaagccgagagaggagcagtacaactccacgtatcgcgttgtctccgtgctgaccgtgctgcatcaggactggctcaacggaaaggagtacaagtgcaaagtgtcgaacaaggcgttgccggcaccaatcgaaaagactatctcgaaagcgaagggacagcccagggagcctcaggtctacacactgcctccaagccgggaagaaatgactaagaaccaagtgtcgctgacttgccttgtgaagggcttttacccctccgacattgccgtcgaatgggaatccaacgggcagcccgagaacaactacaagaccaccccgcctgtgctggacagcgacggcagcttcttcttgtactcgaagctgaccgtggataagtcccggtggcaacagggcaatgtgttctcctgctccgtgatgcacgaagccctccacaaccactacacgcagaagtccctgtcgctttcacccggc

SEQ ID NO: 206 - protein, 218 aa, Homo sapiens
  REGION 1..218: note = misc_feature - hz13-12 D31E LC
DVVMTQSPLSLPVTLGQPASISCRASENVNNYGIGFMNWFQQRPGQSPRLLIYAASRRGSGVPDRFSGSGSGTDFTLKISRVEEEDVGVYYCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 207 - DNA, 654 bp, synthetic construct
  misc_feature 1..654: note = Synthetic: hz13-12 D31E LC DNA sequence
  source 1..654: mol_type = other DNA
gacgtcgtgatgacccagtcgcccctgtcgttgcccgtgactctgggtcagcctgcctcaattagctgtagggccagcgagaacgtgaacaactacggaatcgggttcatgaactggttccagcaacgcccgggacaaagcccccggctgctgatctacgccgcgtcaagacggggttcaggagtgccagatcggttctctggttccggatcgggcaccgacttcacgctgaaaatctcccgcgtggaagaggaggacgtgggagtgtattactgtcagcagtccaaggaagtgccttacaccttcggcggcggaactaaggtcgagattaagaggaccgtggctgctccttccgtgtttatcttcccgccgtccgatgaacagctgaagtccgggactgccagcgtcgtgtgcctgctcaacaacttttacccgcgggaggccaaagtgcagtggaaggtcgacaacgcgctccaatccggcaatagccaggaatccgtgaccgagcaggactcgaaggattccacatactccctgtcctcgacccttactctgagcaaggccgactacgagaagcacaaggtctacgcctgcgaagtgacccatcaaggactttccagccccgtgaccaagtccttcaaccgcggcgaatgc

SEQ ID NO: 208 - protein, 118 aa, unidentified
  REGION 1..118: note = mumAb (murine surrogate) VH
EVQLQQPGAELVKPGASVKLSCKASDHTFTTYWIHWVKQRPGRGLEWIGRIGPNSGGSKYNEKFRTKATLTVDKPSSTAYMQLSSLKSEDSAVYFCAFSNYLDYLDYWGQGTTLTVSS

SEQ ID NO: 209 - DNA, 354 bp, synthetic construct
  misc_feature 1..354: note = Synthetic: mumAb VH DNA sequence
  source 1..354: mol_type = other DNA
gaggtccagctgcagcagcctggggctgagcttgtgaagcctggggcttcagtgaagctgtcctgcaaggcttctgaccacaccttcaccacctactggatacactgggtgaagcagaggcctggacgaggccttgagtggattggaaggattggtcctaatagtggtggttctaaatacaatgagaagttcaggaccaaggccacactgactgtagacaaaccctccagcacagcctacatgcagctcagcagcctgaaatctgaggactctgcggtctatttttgtgcctttagtaactacttggactaccttgactactggggccaaggcaccactctcacagtctcctca

SEQ ID NO: 210 - protein, 111 aa, Homo sapiens
  REGION 1..111: note = misc_feature - muAb VL
DIVLTQSPASLAVSLGQRATISCKASQSVDHDGEGYMNWYQQKPGQPPKLLIYAASNLESGIPARFSGSGSGTDFTLNIHPVEEEDAATYYCQQINEDPYTFGGGTKLEIK

SEQ ID NO: 211 - DNA, 333 bp, synthetic construct
  misc_feature 1..333: note = Synthetic: mumAb VL DNA sequence
  source 1..333: mol_type = other DNA
gacattgtgctcacccaatctccagcttctttggctgtgtctcttgggcagagggccaccatctcctgcaaggccagccaaagtgttgatcatgatggtgaaggttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacttattactgtcagcaaattaatgaggatccgtacacgttcggaggggggaccaagctggaaataaaa

SEQ ID NO: 212 - protein, 441 aa, Homo sapiens
  REGION 1..441: note = misc_feature - mumAb HC; mIgG1 D265A
EVQLQQPGAELVKPGASVKLSCKASDHTFTTYWIHWVKQRPGRGLEWIGRIGPNSGGSKYNEKFRTKATLTVDKPSSTAYMQLSSLKSEDSAVYFCAFSNYLDYLDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 213 - DNA, 1323 bp, synthetic construct
  misc_feature 1..1323: note = Synthetic: mumAb HC DNA sequence
  source 1..1323: mol_type = other DNA
gaggtccagctgcagcagcctggggctgagcttgtgaagcctggggcttcagtgaagctgtcctgcaaggcttctgaccacaccttcaccacctactggatacactgggtgaagcagaggcctggacgaggccttgagtggattggaaggattggtcctaatagtggtggttctaaatacaatgagaagttcaggaccaaggccacactgactgtagacaaaccctccagcacagcctacatgcagctcagcagcctgaaatctgaggactctgcggtctatttttgtgcctttagtaactacttggactaccttgactactggggccaaggcaccactctcacagtctcctcagctaaaacgacacccccatctgtctatccgctggcccctggatccgctgcccaaactaactccatggtgaccctgggatgcctggtcaagggctatttccctgagccagtgacagtgacctggaactctggctccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagccatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggt

SEQ ID NO: 214 - protein, 218 aa, Homo sapiens
  REGION 1..218: note = misc_feature - mumAb LC; mIgG1 D265A
DIVLTQSPASLAVSLGQRATISCKASQSVDHDGEGYMNWYQQKPGQPPKLLIYAASNLESGIPARFSGSGSGTDFTLNIHPVEEEDAATYYCQQINEDPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 215 - DNA, 654 bp, synthetic construct
  misc_feature 1..654: note = Synthetic: mumAb LC DNA sequence
  source 1..654: mol_type = other DNA
gacattgtgctcacccaatctccagcttctttggctgtgtctcttgggcagagggccaccatctcctgcaaggccagccaaagtgttgatcatgatggtgaaggttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacttattactgtcagcaaattaatgaggatccgtacacgttcggaggggggaccaagctggaaataaaacgggctgatgctgcaccaactgtatccatcttcccaccatccagtgagcaattgacatctggaggtgcctcagtcgtgtgcttcttgaacaacttctaccccaaagacatcaatgtcaagtggaagattgatggcagtgaacgacaaaatggcgtcctgaacagttggaccgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 216 - protein, 11 aa, Homo sapiens
  REGION 1..11: note = misc_feature - PAD4 assay substrate
TSTGGRQGSHH

SEQ ID NO: 217 - protein, 27 aa, Homo sapiens
  REGION 1..27: note = misc_feature - Clone 13 s primary HDX epitope
FEGIKKKKQQKIKNILSNKTLREHNSF

SEQ ID NO: 218 - protein, 14 aa, Homo sapiens
  REGION 1..14: note = misc_feature - Clone 13 s secondary HDX epitope
FQEQQNEGHGEALL

SEQ ID NO: 219 - protein, 19 aa, Homo sapiens
  REGION 1..19: note = misc_feature - Clone 20 s primary HDX epitope
INDFFTYHIRHGEVHCGTN

SEQ ID NO: 220 - protein, 11 aa, Homo sapiens
  REGION 1..11: note = misc_feature - Clone 20 s secondary HDX epitope
CPEEENMDDQW

SEQ ID NO: 221 - protein, 7 aa, Homo sapiens
  REGION 1..7: note = misc_feature - Clone 13 HC paratope region 1
FNIKDHF

SEQ ID NO: 222 - protein, 15 aa, Homo sapiens
  REGION 1..15: note = misc_feature - Clone 13 HC paratope region 2
FYCAGYGNYEGAMDY

SEQ ID NO: 223 - protein, 12 aa, Homo sapiens
  REGION 1..12: note = misc_feature - Clone 13 LC paratope region 1
SENVNNYGIGFM

SEQ ID NO: 224 - protein, 8 aa, Homo sapiens
  REGION 1..8: note = misc_feature - Clone 13 LC paratope region 2
VCFLNNFY

SEQ ID NO: 225 - protein, 7 aa, Homo sapiens
  REGION 1..7: note = misc_feature - Hz13-5 D31E HC paratope region 1
FNIKEHF

SEQ ID NO: 226 - protein, 229 aa, Homo sapiens
  REGION 1..229: note = misc_feature - PAD4 clone 13 Fab with his tag; HC
EVLLQQSGAELVKPGASVRLSCTSSDFNIKDHFIHWVKQRTEQGLEWIGRIDPEDDETKYAPKFQDKVTITADTSSNTAYLQLSSLTSEDTAVFYCAGYGNYEGAMDYWGQGTSVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGGHHHHHH

SEQ ID NO: 227 - protein, 223 aa, Homo sapiens
  REGION 1..223: note = misc_feature - PAD4 clone 13 Fab; HC
EVLLQQSGAELVKPGASVRLSCTSSDFNIKDHFIHWVKQRTEQGLEWIGRIDPEDDETKYAPKFQDKVTITADTSSNTAYLQLSSLTSEDTAVFYCAGYGNYEGAMDYWGQGTSVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGG

SEQ ID NO: 228 - protein, 218 aa, Homo sapiens
  REGION 1..218: note = misc_feature - PAD4 clone 13 Fab; LC
DIVLTQSPTSLGVSPGQRATLSCRASENVNNYGIGFMNWFQQKPGQPPKLLIYAASRRGSGVPARFSGSGSGTDFSLNIHPMEEDDTAMYFCQQSKEVPYTFGGGTNLEIRRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 229 - protein, 231 aa, Homo sapiens
  REGION 1..231: note = misc_feature - PAD4 clone 20 Fab with his tag; HC; mIgG1 CH1 and mouse kappa constant region
QVQLQQSGAELARPGASVKLSCKATGYTFTNYGISWVKQRTGQGLEWIGEIYPRSGNTYHNEKFKDKATLTADKSSSTAYMEFRSLTSEDSAVYFCARSMITTRYWYFDVWGKGTTVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGGHHHHHH

SEQ ID NO: 230 - protein, 225 aa, Homo sapiens
  REGION 1..225: note = misc_feature - PAD4 clone 20 Fab; HC; mIgG1 CH1 and mouse kappa constant region
QVQLQQSGAELARPGASVKLSCKATGYTFTNYGISWVKQRTGQGLEWIGEIYPRSGNTYHNEKFKDKATLTADKSSSTAYMEFRSLTSEDSAVYFCARSMITTRYWYFDVWGKGTTVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGG

SEQ ID NO: 231 - protein, 218 aa, Homo sapiens
  REGION 1..218: note = misc_feature - Clone 20 Fab; LC; mIgG1 CH1 and mouse kappa constant region
NIVLTQSPASLAVSLGQRATLSCRASESVDTYGNSFMHWYQQKPGQPPKLLIYLASNLESGVPARFSGSGSRTDFTLTIDPVEADDAATYYCQQNNEDPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 232 - protein, 18 aa, Homo sapiens
  REGION 1..18: note = misc_feature - Arginine-coated linear peptide
SHQESTRGKSKGKAAAAA

SEQ ID NO: 233 - protein, 119 aa, Homo sapiens
  REGION 1..119: note = misc_feature - hz13-12 D31E VH
EVQLVQSGAEVKKPGATVKISCTSSDFNIKEHFIHWVQQAPGKGLEWMGRIDPEDDETKYAPKFQDRVTITADTSTDTAYMELSSLRSEDTAVYYCAGYGNYEGAMDYWGQGTTVTVSS

SEQ ID NO: 234 - protein, 111 aa, Homo sapiens; identical to SEQ ID NO: 52
  REGION 1..111: note = misc_feature - hz13-12 D31E VL

SEQ ID NO: 235 - protein, 8 aa, Homo sapiens
  REGION 1..8: note = misc_feature - Human LC constant region fragment corresponding to Clone 13 LC paratope region 2
VCLLNNFY

SEQ ID NO: 236 - protein, 12 aa, Homo sapiens
  REGION 1..12: note = misc_feature - Exemplary PRG4 peptide sequence (human)
AITTRSGQTLSK

SEQ ID NO: 237 - protein, 18 aa, Homo sapiens
  REGION 1..18: note = misc_feature - Exemplary FGA peptide sequence (human)
QFTSSTSYNRGDSTFESK

SEQ ID NO: 238 - protein, 21 aa, Homo sapiens
  REGION 1..21: note = misc_feature - ExemplaryITIH4 peptide sequence(human)
QLGLPGPPDVPDHAAYHPFRR

SEQ ID NO: 239 - protein, 16 aa, Homo sapiens
  REGION 1..16: note = misc_feature - Exemplary AMBP peptide sequence (human)
GPCRAFIQLWAFDAVK

SEQ ID NO: 240 - protein, 16 aa, Homo sapiens
  REGION 1..16: note = misc_feature - Exemplary GSN peptide sequence (human)
ATASRGASQAGAPQGR

SEQ ID NO: 241 - protein, 17 aa, Homo sapiens
  REGION 1..17: note = misc_feature - Exemplary FGA peptide sequence (human)
ESSSHHPGIAEFPSRGK

SEQ ID NO: 242 - protein, 18 aa, Homo sapiens
  REGION 1..18: note = misc_feature - Exemplary C3 peptide sequence (human)
SGQSEDRQPVPGQQMTLK

SEQ ID NO: 243 - protein, 24 aa, Homo sapiens
  REGION 1..24: note = misc_feature - Exemplary C3 peptide sequence (human)
ASHLGLARSNLDEDIIAEENIVSR

SEQ ID NO: 244 - protein, 15 aa, Homo sapiens
  REGION 1..15: note = misc_feature - Exemplary A2M peptide sequence (human)
VGFYESDVMGRGHAR

SEQ ID NO: 245 - protein, 14 aa, Homo sapiens
  REGION 1..14: note = misc_feature - Exemplary HP peptide sequence (human)
LRTEGDGVYTLNDK

SEQ ID NO: 246 - protein, 13 aa, Homo sapiens
  REGION 1..13: note = misc_feature - Exemplary TF peptide sequence (human)
DLLFRDDTVCLAK

(19) United States
(12) Patent Application Publication
(10) Pub. No.: US 2024/0228664 A9
(43) Pub. Date: Jul. 11, 2024

(54) Antibodies Binding to Human PAD4 and Uses Thereof

(71) Applicant: Bristol-Myers Squibb Company, Princeton, NJ (US)

(72) Inventors: Lin Hui Su, Cambridge, MA (US); Ekaterina Deyanova, Princeton, NJ (US); Burce Ergel Gurbuzbalaban, Princeton, NJ (US); Mariana Nacht, Belmont, MA (US); Samantha Elaine Pace, Princeton, NJ (US); Yun Wang, Princeton, NJ (US); Qing Xiao, Cambridge, MA (US); Ramakrishna Chandran, Princeton, NJ (US); Shailesh Dudhgaonkar, Bangalore (IN); Michael Louis Doyle, Princeton, NJ (US); Michael Gilman, Waban, MA (US); Richard Huang, Princeton, NJ (US); Akbar Nayeem, Princeton, NJ (US); Alok Sharma, Princeton, NJ (US); Qihong Zhao, Princeton, NJ (US)

(73) Assignee: Bristol-Myers Squibb Company, Princeton, NJ (US)

(21) Appl. No.: 18/357,007

(22) Filed: Jul. 21, 2023

Related U.S. Application Data
(60) Provisional application No. 63/397,698, filed on Aug. 12, 2022.
(60) Provisional application No. 63/369,184, filed on Jul. 22, 2022.

(51) Int. Cl. C07K 16/40; A01N 1/02; A61K 45/06; A61K 47/68; A61K 49/00; A61P 19/02; A61P 37/06

(52) U.S. Cl. CPC C07K 16/40; A01N 1/0226; A61K 45/06; A61K 47/6871; A61K 49/0002; A61P 19/02; A61P 37/06

(57) ABSTRACT

The present application relates to particular anti-PAD4 (peptidyl arginine deiminase 4) antibodies, nucleic acids encoding the antibodies, vectors and host cells comprising the nucleic acids, and methods of making and using the antibodies.

### CROSS-REFERENCE TO RELATED APPLICATIONS

This application claims the benefit of priority to U.S. Provisional Application No. 63/369,184, filed Jul. 22, 2022, and to U.S. Provisional Application No. 63/397,698, filed Aug. 12, 2022, both of which are incorporated by reference herein in their entirety.

### SEQUENCE LISTING

The present application contains a Sequence Listing which has been submitted electronically in XML format. Said XML copy, created on Jul. 21, 2023, is named “01275-0066-00PCT.xml” and is 377,402 bytes in size. The information in the electronic format of the sequence listing is incorporated herein by reference in its entirety.

### FIELD

The present application relates to particular anti-PAD4 (peptidyl arginine deiminase 4) antibodies, nucleic acids encoding the antibodies, vectors and host cells comprising the nucleic acids, and methods of making and using the antibodies.

### BACKGROUND

PAD4 (peptidyl arginine deiminase 4) is one of five PAD enzymes in humans (PAD1, 2, 3, 4, and 6) and is found in several immune cell types and acts by converting arginine to citrulline. (Chavanas et al., Gene 330: 19-27 (2004).) Such citrullination is a stress response and may serve as a signal for removal of stressed cells. (Brentville et al., Oncoimmunology 8: e1576490 (2019).) In neutrophils, PAD4 also plays a role in a process called NETosis, by which neutrophils extrude a complex of decondensed chromatin structures containing a DNA scaffold, citrullinated histones, and anti-bacterial neutrophilic granules. (Li et al. J. Exp. Med. 207: 1853-62 (2010).) These extruded complexes are called neutrophil extracellular traps (NET) and, during NETosis, these NETs trap and kill invading microbes as part of the innate immune response. (Chamardani et al., Mol. Cell. Biochem. 477: 673-88 (2022).) A similar process involving monocytes is called METosis and involves formation of monocyte extracellular traps (MET). Moreover, proteins citrullinated by PAD4 become antigenic substrates and are targets for both cellular (i.e., T cell) and humoral (i.e., B cell-derived antibody) adaptive immune responses. (See, e.g., Curran et al. Nat. Rev. Rheumatol. 16: 301-15 (2020); Brentville et al.) Thus, PAD4 activity may lead to generation of anti-citrullinated protein antibodies (ACPA).

A variety of data suggest that PAD4 plays a role in diseases such as rheumatoid arthritis (RA), lupus (including systemic lupus erythematosus (SLE), lupus nephritis, vasculitis (including anti-neutrophilic cytoplasmic antibody (ANCA)-associated vasculitis, inflammatory bowel disease (IBD) (including ulcerative colitis and Crohn's disease), thrombosis (e.g., venous thrombosis), antiphospholipid antibody syndrome, cystic fibrosis, and cancers. (See, e.g., Curran et al.; Yadav et al., J. Cyst. Fibros. 18: 636-45 (2018); Wang et al., Front. Immunol. 13: 895216 (2022); Fresneda Alarcon et al. Frong. Immunol. 12: 649693 (2021); Weeding et al., Clin. Immunol. 196: 110-116 (2018); Xu et al., Chinese J. Microbiology and Immunology 12: 115-121 (2020); Yoshida et al., Clin. Kidney J. 6: 308-12 (2013); O'Sullivan et al., Rheumatology, 58(Suppl. 2): kez061.024 (2019); Pan et al., Authorea Preprints, 2021, DOI: 10.22541/au.161590650.07168461/v1; D. Zhu et al., Pharmaceutics, 8: 14(11): 2414 (2022).) For example, rheumatoid arthritis (RA) is a major autoimmune disorder that involves both tissue (i.e., joint and non-joint) and systemic inflammation. Progression of RA is associated with increasing levels of auto-antibodies, and about 70% of RA patients are positive for anti-citrullinated protein antibodies (ACPA). ACPA appear, for example, in the lung and blood of those at risk for RA, and high ACPA levels are associated with severe RA symptoms, such as bone erosion, and certain co-morbidities, such as cardiovascular disease. In addition, presence of ACPA worsened RA symptoms in a murine model of spontaneous arthritis.

ACPA recognize neo-antigens, particularly those from proteins citrullinated by PAD enzymes. PAD4 is the dominant PAD enzyme found in synovial tissues. For example, PAD4 can mediate ACPA-dependent and ACPA-independent functions important to the pathogenesis of RA and other diseases. In addition, about 15% of RA patients have antibodies that activate PAD4. Presence of such PAD4-activating antibodies correlates with severe joint erosive disease. On the genetic level, single-nucleotide polymorphisms (SNP) in the PADI4 gene encoding PAD4 have been identified that contribute to a susceptibility haplotype for RA. (Suzuki et al., Nat. Genet. 34: 395-402 (2003).) Furthermore, epigenetic changes at the promoter region of PADI4 are associated with disease activity with lower methylation in RA patients compared to healthy individuals. (Kolarz et al., Clin. Med. 9(7): 2049 (2020).) RA patients carrying the susceptibility haplotype for PADI4 were found to have higher titers of anti-citrullinated peptide antibodies (ACPA) than RA patients without the haplotype. (Reyes-Castillo et al., Clin. Exp. Immunol. 182(2): 119-31 (2015).)

Accordingly, blocking the activity of PAD4 may be useful in treating RA, and in treating subjects at risk for developing RA, as well as other inflammatory and autoimmune diseases or other diseases associated with NETosis, METosis, presence of anti-citrullinated protein antibodies (ACPA), increased PAD4 expression, or increased PAD4 activity such as increased citrullination of polypeptides.

The present disclosure provides anti-PAD4 antibodies that inhibit the activity of PAD4, nucleic acids encoding the antibodies, vectors and host cells comprising the antibodies, and methods of making and using the antibodies.

### SUMMARY

The present disclosure relates to particular anti-PAD4 antibodies, nucleic acids encoding the antibodies, vectors and host cells comprising the nucleic acids, and methods of both making and using the antibodies. For example, embodiments of the disclosure include the following:
1. An isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of SEQ ID NO: 4 or 62 and an HCDR3 comprising the amino acid sequence of SEQ ID NO: 6; and a light chain variable region (VL) comprising a light chain complementarity determining region 1 (LCDR1) comprising the amino acid sequence of SEQ ID NO: 7.
2. An isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of SEQ ID NO: 4 or 62, an HCDR2 comprising the amino acid sequence of SEQ ID NO: 5, and an HCDR3 comprising the amino acid sequence of SEQ ID NO: 6; and a light chain variable region (VL) comprising a light chain complementarity determining region 1 (LCDR1) comprising the amino acid sequence of SEQ ID NO: 7, an LCDR2 comprising the amino acid sequence of SEQ ID NO: 8, and an LCDR3 comprising the amino acid sequence of SEQ ID NO: 9.
3. An isolated antibody that specifically binds to protein arginine deiminase 4 (PAD4), wherein the antibody comprises a heavy chain variable region (VH) comprising the amino acid sequence of SEQ ID NO: 221 or 225 and the amino acid sequence of SEQ ID NO: 222; and a light chain variable region (VL) comprising the amino acid sequence of SEQ ID NO: 223.
4. An isolated antibody that specifically binds to protein arginine deiminase 4 (PAD4), wherein the antibody comprises a heavy chain variable region (VH) comprising the amino acid sequence of SEQ ID NO: 221 or 225 and the amino acid sequence of SEQ ID NO: 222; and a light chain variable region (VL) comprising the amino acid sequence of SEQ ID NO: 223, and further wherein the antibody comprises a light chain constant region comprising the amino acid sequence of SEQ ID NO: 224 or 235.
5. The isolated antibody of any one of embodiments 1-4, wherein the antibody comprises a VH that is at least 90%, at least 95%, at least 97%, or at least 99% identical to the amino acid sequence of any one of SEQ ID NOs: 10, 14, 18, 22, 26, 30, 34, 38, 42, 46, 50, 54, 58, or 68; and wherein the antibody comprises a VL that is at least 90%, at least 95%, at least 97%, or at least 99% identical to the amino acid sequence of any one of SEQ ID NOs: 12, 16, 20, 24, 28, 32, 36, 40, 44, 48, 52, 56, 60, or 70.
6. The isolated antibody of any one of embodiments 1-4, wherein the antibody comprises a VH comprising the amino acid sequence of any one of SEQ ID Nos: 10, 14, 18, 22, 26, 30, 34, 38, 42, 46, 50, 54, 58, or 68 modified by 1-10 amino acid substitutions, 1-5 amino acid substitutions, or 1-3 amino acid substitutions; and wherein the antibody comprises a VL comprising the amino acid sequence of any one of SEQ ID Nos: 12, 16, 20, 24, 28, 32, 36, 40, 44, 48, 52, 56, 60, or 70 modified by 1-10 amino acid substitutions, 1-5 amino acid substitutions, or 1-3 amino acid substitutions.
7. The isolated antibody of any one of embodiments 1-6, wherein the heavy chain variable region comprises a glycine at Kabat position 94 (which corresponds to position 98 of SEQ ID NO: 10).
8. The isolated antibody of any one of embodiments 1-7, wherein the antibody comprises a VH comprising the amino acid sequence of any one of SEQ ID Nos: 10, 14, 18, 22, 26, 30, 34, 38, 42, 46, 50, 54, 58, or 68.
9. The isolated antibody of any one of embodiments 1-8, wherein the antibody comprises a VL comprising the amino acid sequence of any one of SEQ ID Nos: 12, 16, 20, 24, 28, 32, 36, 40, 44, 48, 52, 56, 60, or 70.
10. An isolated antibody that specifically binds to protein arginine deiminase 4 (PAD4), wherein the antibody comprises a VH comprising the amino acid sequence of SEQ ID NO: 10 and a VL comprising the amino acid sequence of SEQ ID NO: 12.
11. An isolated antibody that specifically binds to protein arginine deiminase 4 (PAD4), wherein the antibody comprises a VH comprising the amino acid sequence of SEQ ID NO: 14 and a VL comprising the amino acid sequence of SEQ ID NO: 16.
12. An isolated antibody that specifically binds to protein arginine deiminase 4 (PAD4), wherein the antibody comprises a VH comprising the amino acid sequence of SEQ ID NO: 18 and a VL comprising the amino acid sequence of SEQ ID NO: 20.
13. An isolated antibody that specifically binds to protein arginine deiminase 4 (PAD4), wherein the antibody comprises a VH comprising the amino acid sequence of SEQ ID NO: 22 and a VL comprising the amino acid sequence of SEQ ID NO: 24.
14. An isolated antibody that specifically binds to protein arginine deiminase 4 (PAD4), wherein the antibody comprises a VH comprising the amino acid sequence of SEQ ID NO: 26 and a VL comprising the amino acid sequence of SEQ ID NO: 28.
15. An isolated antibody that specifically binds to protein arginine deiminase 4 (PAD4), wherein the antibody comprises a VH comprising the amino acid sequence of SEQ ID NO: 30 and a VL comprising the amino acid sequence of SEQ ID NO: 32;
16. An isolated antibody that specifically binds to protein arginine deiminase 4 (PAD4), wherein the antibody comprises a VH comprising the amino acid sequence of SEQ ID NO: 34 and a VL comprising the amino acid sequence of SEQ ID NO: 36.
17. An isolated antibody that specifically binds to protein arginine deiminase 4 (PAD4), wherein the antibody comprises a VH comprising the amino acid sequence of SEQ ID NO: 38 and a VL comprising the amino acid sequence of SEQ ID NO: 40.
18. An isolated antibody that specifically binds to protein arginine deiminase 4 (PAD4), wherein the antibody comprises a VH comprising the amino acid sequence of SEQ ID NO: 42 and a VL comprising the amino acid sequence of SEQ ID NO: 44.
19. An isolated antibody that specifically binds to protein arginine deiminase 4 (PAD4), wherein the antibody comprises a VH comprising the amino acid sequence of SEQ ID NO: 46 and a VL comprising the amino acid sequence of SEQ ID NO: 48.
20. An isolated antibody that specifically binds to protein arginine deiminase 4 (PAD4), wherein the antibody comprises a VH comprising the amino acid sequence of SEQ ID NO: 50 and a VL comprising the amino acid sequence of SEQ ID NO: 52.
21. An isolated antibody that specifically binds to protein arginine deiminase 4 (PAD4), wherein the antibody comprises a VH comprising the amino acid sequence of SEQ ID NO: 54 and a VL comprising the amino acid sequence of SEQ ID NO: 56.
22. An isolated antibody that specifically binds to protein arginine deiminase 4 (PAD4), wherein the antibody comprises a VH comprising the amino acid sequence of SEQ ID NO: 58 and a VL comprising the amino acid sequence of SEQ ID NO: 60.
23. An isolated antibody that specifically binds to protein arginine deiminase 4 (PAD4), wherein the antibody comprises a VH comprising the amino acid sequence of SEQ ID NO: 68 and a VL comprising the amino acid sequence of SEQ ID NO: 70.
24. An isolated antibody that specifically binds to protein arginine deiminase 4 (PAD4), wherein the antibody comprises a VH comprising the amino acid sequence of SEQ ID NO: 233 and a VL comprising the amino acid sequence of SEQ ID NO: 234.
25. The isolated antibody of any one of embodiments 1-23, wherein the antibody binds to an epitope on PAD4 comprising SEQ ID NO: 217 and SEQ ID NO: 218.
26. An isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of SEQ ID NO: 72, an HCDR2 comprising the amino acid sequence of SEQ ID NO: 73, and an HCDR3 comprising the amino acid sequence of SEQ ID NO: 74; and a light chain variable region (VL) comprising a light chain complementarity determining region 1 (LCDR1) comprising the amino acid sequence of SEQ ID NO: 75, an LCDR2 comprising the amino acid sequence of SEQ ID NO: 76, and an LCDR3 comprising the amino acid sequence of SEQ ID NO: 77.
27. The isolated antibody of embodiment 26, wherein the antibody comprises a VH that is at least 90%, at least 95%, at least 97%, or at least 99% identical to the amino acid sequence of any one of SEQ ID NOs: 78, 82, 86, 90, 94, 98, 102, 106, 110, 114, 118, 122, 126, 130, or 134; and wherein the antibody comprises a VL that is at least 90%, at least 95%, at least 97%, or at least 99% identical to the amino acid sequence of any one of SEQ ID NOs: 80, 84, 88, 92, 96, 100, 104, 108, 112, 116, 120, 124, 128, 132, or 136.
28. The isolated antibody of embodiment 26, wherein the antibody comprises a VH comprising the amino acid sequence of any one of SEQ ID Nos: 78, 82, 86, 90, 94, 98, 102, 106, 110, 114, 118, 122, 126, 130, or 134 modified by 1-10 amino acid substitutions, 1-5 amino acid substitutions, or 1-3 amino acid substitutions; and wherein the antibody comprises a VL comprising the amino acid sequence of any one of SEQ ID Nos: 80, 84, 88, 92, 96, 100, 104, 108, 112, 116, 120, 124, 128, 132, or 136 modified by 1-10 amino acid substitutions, 1-5 amino acid substitutions, or 1-3 amino acid substitutions.
29. The isolated antibody of embodiment 25, 26, or 27, wherein the antibody comprises a VH comprising the amino acid sequence of any one of SEQ ID Nos: 78, 82, 86, 90, 94, 98, 102, 106, 110, 114, 118, 122, 126, 130, or 134.
30. The isolated antibody of any one of embodiments 26-29, wherein the antibody comprises a VL comprising the amino acid sequence of any one of SEQ ID Nos: 80, 84, 88, 92, 96, 100, 104, 108, 112, 116, 120, 124, 128, 132, or 136.
31. An isolated antibody that specifically binds to protein arginine deiminase 4 (PAD4), wherein the antibody comprises a VH comprising the amino acid sequence of SEQ ID NO: 78 and a VL comprising the amino acid sequence of SEQ ID NO: 80
32. An isolated antibody that specifically binds to protein arginine deiminase 4 (PAD4), wherein the antibody comprises a VH comprising the amino acid sequence of SEQ ID NO: 82 and a VL comprising the amino acid sequence of SEQ ID NO: 84.
33. An isolated antibody that specifically binds to protein arginine deiminase 4 (PAD4), wherein the antibody comprises a VH comprising the amino acid sequence of SEQ ID NO: 86 and a VL comprising the amino acid sequence of SEQ ID NO: 88.
34. An isolated antibody that specifically binds to protein arginine deiminase 4 (PAD4), wherein the antibody comprises a VH comprising the amino acid sequence of SEQ ID NO: 90 and a VL comprising the amino acid sequence of SEQ ID NO: 92.
35. An isolated antibody that specifically binds to protein arginine deiminase 4 (PAD4), wherein the antibody comprises a VH comprising the amino acid sequence of SEQ ID NO: 94 and a VL comprising the amino acid sequence of SEQ ID NO: 96.
36. An isolated antibody that specifically binds to protein arginine deiminase 4 (PAD4), wherein the antibody comprises a VH comprising the amino acid sequence of SEQ ID NO: 98 and a VL comprising the amino acid sequence of SEQ ID NO: 100.
37. An isolated antibody that specifically binds to protein arginine deiminase 4 (PAD4), wherein the antibody comprises a VH comprising the amino acid sequence of SEQ ID NO: 102 and a VL comprising the amino acid sequence of SEQ ID NO: 104.
38. An isolated antibody that specifically binds to protein arginine deiminase 4 (PAD4), wherein the antibody comprises a VH comprising the amino acid sequence of SEQ ID NO: 106 and a VL comprising the amino acid sequence of SEQ ID NO: 108.
39. An isolated antibody that specifically binds to protein arginine deiminase 4 (PAD4), wherein the antibody comprises a VH comprising the amino acid sequence of SEQ ID NO: 110 and a VL comprising the amino acid sequence of SEQ ID NO: 112.
40. An isolated antibody that specifically binds to protein arginine deiminase 4 (PAD4), wherein the antibody comprises a VH comprising the amino acid sequence of SEQ ID NO: 114 and a VL comprising the amino acid sequence of SEQ ID NO: 116.
41. An isolated antibody that specifically binds to protein arginine deiminase 4 (PAD4), wherein the antibody comprises a VH comprising the amino acid sequence of SEQ ID NO: 118 and a VL comprising the amino acid sequence of SEQ ID NO: 120.
42. An isolated antibody that specifically binds to protein arginine deiminase 4 (PAD4), wherein the antibody comprises a VH comprising the amino acid sequence of SEQ ID NO: 122 and a VL comprising the amino acid sequence of SEQ ID NO: 124.
43. An isolated antibody that specifically binds to protein arginine deiminase 4 (PAD4), wherein the antibody comprises a VH comprising the amino acid sequence of SEQ ID NO: 126 and a VL comprising the amino acid sequence of SEQ ID NO: 128.
44. An isolated antibody that specifically binds to protein arginine deiminase 4 (PAD4), wherein the antibody comprises a VH comprising the amino acid sequence of SEQ ID NO: 130 and a VL comprising the amino acid sequence of SEQ ID NO: 132.
45. An isolated antibody that specifically binds to protein arginine deiminase 4 (PAD4), wherein the antibody comprises a VH comprising the amino acid sequence of SEQ ID NO: 134 and a VL comprising the amino acid sequence of SEQ ID NO: 136.
46. The isolated antibody of any one of embodiments 26-45, wherein the antibody binds to an epitope on PAD4 comprising SEQ ID NO: 219 and SEQ ID NO: 220.
47. An isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of:
   a. positions 26-35 of SEQ ID NO: 138, an HCDR2 comprising the amino acid sequence of positions 50-66 of SEQ ID NO: 138, and an HCDR3 comprising the amino acid sequence of positions 99-108 of SEQ ID NO: 138;
   b. positions 26-35 of SEQ ID NO: 140, an HCDR2 comprising the amino acid sequence of positions 50-66 of SEQ ID NO: 140, and an HCDR3 comprising the amino acid sequence of positions 99-108 of SEQ ID NO: 140;
   c. positions 26-35 of SEQ ID NO: 142, an HCDR2 comprising the amino acid sequence of positions 50-66 of SEQ ID NO: 142, and an HCDR3 comprising the amino acid sequence of positions 99-108 of SEQ ID NO: 142;
   d. positions 26-35 of SEQ ID NO: 144, an HCDR2 comprising the amino acid sequence of positions 50-66 of SEQ ID NO: 144, and an HCDR3 comprising the amino acid sequence of positions 99-108 of SEQ ID NO: 144;
   e. positions 26-35 of SEQ ID NO: 146, an HCDR2 comprising the amino acid sequence of positions 50-66 of SEQ ID NO: 146, and an HCDR3 comprising the amino acid sequence of positions 99-108 of SEQ ID NO: 146;
   f. positions 26-35 of SEQ ID NO: 148, an HCDR2 comprising the amino acid sequence of positions 50-66 of SEQ ID NO: 148, and an HCDR3 comprising the amino acid sequence of positions 99-108 of SEQ ID NO: 148;
   g. positions 26-35 of SEQ ID NO: 150, an HCDR2 comprising the amino acid sequence of positions 50-66 of SEQ ID NO: 150, and an HCDR3 comprising the amino acid sequence of positions 99-108 of SEQ ID NO: 150;
   h. positions 26-35 of SEQ ID NO: 152, an HCDR2 comprising the amino acid sequence of positions 50-66 of SEQ ID NO: 152, and an HCDR3 comprising the amino acid sequence of positions 99-108 of SEQ ID NO: 152;
   i. positions 26-35 of SEQ ID NO: 154, an HCDR2 comprising the amino acid sequence of positions 50-66 of SEQ ID NO: 154, and an HCDR3 comprising the amino acid sequence of positions 99-108 of SEQ ID NO: 154;
   j. positions 26-35 of SEQ ID NO: 156, an HCDR2 comprising the amino acid sequence of positions 50-66 of SEQ ID NO: 156, and an HCDR3 comprising the amino acid sequence of positions 99-108 of SEQ ID NO: 156;
   k. positions 26-35 of SEQ ID NO: 158, an HCDR2 comprising the amino acid sequence of positions 50-66 of SEQ ID NO: 158, and an HCDR3 comprising the amino acid sequence of positions 99-108 of SEQ ID NO: 158;
   l. positions 26-35 of SEQ ID NO: 160, an HCDR2 comprising the amino acid sequence of positions 50-66 of SEQ ID NO: 160, and an HCDR3 comprising the amino acid sequence of positions 99-108 of SEQ ID NO: 160;
   m. positions 26-35 of SEQ ID NO: 162, an HCDR2 comprising the amino acid sequence of positions 50-66 of SEQ ID NO: 162, and an HCDR3 comprising the amino acid sequence of positions 99-108 of SEQ ID NO: 162;
   n. positions 26-35 of SEQ ID NO: 164, an HCDR2 comprising the amino acid sequence of positions 50-66 of SEQ ID NO: 164, and an HCDR3 comprising the amino acid sequence of positions 99-108 of SEQ ID NO: 164;
   o. positions 26-35 of SEQ ID NO: 166, an HCDR2 comprising the amino acid sequence of positions 50-66 of SEQ ID NO: 166, and an HCDR3 comprising the amino acid sequence of positions 99-108 of SEQ ID NO: 166;
   p. positions 26-35 of SEQ ID NO: 168, an HCDR2 comprising the amino acid sequence of positions 50-66 of SEQ ID NO: 168, and an HCDR3 comprising the amino acid sequence of positions 99-108 of SEQ ID NO: 168; or
   q. positions 26-35 of SEQ ID NO: 170, an HCDR2 comprising the amino acid sequence of positions 50-66 of SEQ ID NO: 170, and an HCDR3 comprising the amino acid sequence of positions 99-108 of SEQ ID NO: 170; and
      wherein the antibody comprises a light chain variable region (VL) comprising a light chain complementarity determining region 1 (LCDR1) comprising the amino acid sequence of residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170.
48. An isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of:
   a. positions 26-35 of SEQ ID NO: 138, an HCDR2 comprising the amino acid sequence of positions 50-66 of SEQ ID NO: 138, and an HCDR3 comprising the amino acid sequence of positions 99-108 of SEQ ID NO: 138;
   b. positions 26-35 of SEQ ID NO: 140, an HCDR2 comprising the amino acid sequence of positions 50-66 of SEQ ID NO: 140, and an HCDR3 comprising the amino acid sequence of positions 99-108 of SEQ ID NO: 140;
   c. positions 26-35 of SEQ ID NO: 142, an HCDR2 comprising the amino acid sequence of positions 50-66 of SEQ ID NO: 142, and an HCDR3 comprising the amino acid sequence of positions 99-108 of SEQ ID NO: 142;
   d. positions 26-35 of SEQ ID NO: 144, an HCDR2 comprising the amino acid sequence of positions 50-66 of SEQ ID NO: 144, and an HCDR3 comprising the amino acid sequence of positions 99-108 of SEQ ID NO: 144;
   e. positions 26-35 of SEQ ID NO: 146, an HCDR2 comprising the amino acid sequence of positions 50-66 of SEQ ID NO: 146, and an HCDR3 comprising the amino acid sequence of positions 99-108 of SEQ ID NO: 146;
   f. positions 26-35 of SEQ ID NO: 148, an HCDR2 comprising the amino acid sequence of positions 50-66 of SEQ ID NO: 148, and an HCDR3 comprising the amino acid sequence of positions 99-108 of SEQ ID NO: 148;
   g. positions 26-35 of SEQ ID NO: 150, an HCDR2 comprising the amino acid sequence of positions 50-66 of SEQ ID NO: 150, and an HCDR3 comprising the amino acid sequence of positions 99-108 of SEQ ID NO: 150;
   h. positions 26-35 of SEQ ID NO: 152, an HCDR2 comprising the amino acid sequence of positions 50-66 of SEQ ID NO: 152, and an HCDR3 comprising the amino acid sequence of positions 99-108 of SEQ ID NO: 152;
   i. positions 26-35 of SEQ ID NO: 154, an HCDR2 comprising the amino acid sequence of positions 50-66 of SEQ ID NO: 154, and an HCDR3 comprising the amino acid sequence of positions 99-108 of SEQ ID NO: 154;
   j. positions 26-35 of SEQ ID NO: 156, an HCDR2 comprising the amino acid sequence of positions 50-66 of SEQ ID NO: 156, and an HCDR3 comprising the amino acid sequence of positions 99-108 of SEQ ID NO: 156;
   k. positions 26-35 of SEQ ID NO: 158, an HCDR2 comprising the amino acid sequence of positions 50-66 of SEQ ID NO: 158, and an HCDR3 comprising the amino acid sequence of positions 99-108 of SEQ ID NO: 158;
   l. positions 26-35 of SEQ ID NO: 160, an HCDR2 comprising the amino acid sequence of positions 50-66 of SEQ ID NO: 160, and an HCDR3 comprising the amino acid sequence of positions 99-108 of SEQ ID NO: 160;
   m. positions 26-35 of SEQ ID NO: 162, an HCDR2 comprising the amino acid sequence of positions 50-66 of SEQ ID NO: 162, and an HCDR3 comprising the amino acid sequence of positions 99-108 of SEQ ID NO: 162;
   n. positions 26-35 of SEQ ID NO: 164, an HCDR2 comprising the amino acid sequence of positions 50-66 of SEQ ID NO: 164, and an HCDR3 comprising the amino acid sequence of positions 99-108 of SEQ ID NO: 164;
   o. positions 26-35 of SEQ ID NO: 166, an HCDR2 comprising the amino acid sequence of positions 50-66 of SEQ ID NO: 166, and an HCDR3 comprising the amino acid sequence of positions 99-108 of SEQ ID NO: 166; or
   p. positions 26-35 of SEQ ID NO: 168, an HCDR2 comprising the amino acid sequence of positions 50-66 of SEQ ID NO: 168, and an HCDR3 comprising the amino acid sequence of positions 99-108 of SEQ ID NO: 168; and
      wherein the antibody comprises a light chain variable region (VL) comprising a light chain complementarity determining region 1 (LCDR1) comprising the amino acid sequence of residues 24-38 of SEQ ID NO: 172, an LCDR2 comprising the amino acid sequence of residues 54-60 of SEQ ID NO: 172, and an LCDR3 comprising the amino acid sequence of residues 93-101 of SEQ ID NO: 172.
49. An isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of positions 26-35 of SEQ ID NO: 168, an HCDR2 comprising the amino acid sequence of positions 50-66 of SEQ ID NO: 168, and an HCDR3 comprising the amino acid sequence of positions 99-108 of SEQ ID NO: 168; and wherein the antibody comprises a light chain variable region (VL) comprising a light chain complementarity determining region 1 (LCDR1) comprising the amino acid sequence of residues 24-38 of SEQ ID NO: 172, an LCDR2 comprising the amino acid sequence of residues 54-60 of SEQ ID NO: 172, and an LCDR3 comprising the amino acid sequence of residues 93-101 of SEQ ID NO: 172.
50. The isolated antibody of embodiment 49, wherein the antibody binds to PAD4 at pH 6.0 with lower affinity compared to an antibody comprising a VH comprising the amino acid sequence of SEQ ID NO: 30 and a VL comprising the amino acid sequence of SEQ ID NO: 32.
51. The isolated antibody of any one of embodiments 1-50, which is an IgA, IgG, or IgM antibody.
52. The isolated antibody of any one of embodiments 1-50, which is an IgG antibody, such as a human IgG1, IgG2, IgG3, or IgG4 antibody or a murine IgG1 or IgG2 antibody.
53. The isolated antibody of embodiment 52, wherein the antibody comprises a wild-type, human IgG1, IgG2, or IgG4 heavy chain constant region.
54. The isolated antibody of any one of embodiments 1-50, wherein the antibody comprises a human IgG1 or IgG2 heavy chain constant region comprising an amino acid sequence at least 90%, at least 95%, at least 97%, or at least 99% identical to the amino acid sequence of any one of SEQ ID Nos: 174, 176, 178, 180, 182, 184, 186, 188, 190, or 192; and/or wherein the antibody comprises a light chain constant region comprising an amino acid sequence at least 90%, at least 95%, at least 97%, or at least 99% identical to the amino acid sequence of SEQ ID NO: 194.
55. The isolated antibody of any one of embodiments 1-50, wherein the antibody comprises a human IgG1 or IgG2 heavy chain constant region comprising an amino acid sequence modified by 1-10 amino acid substitutions, 1-5 amino acid substitutions, or 1-3 amino acid substitutions compared to the amino acid sequence of any one of SEQ ID Nos: 174, 176, 178, 180, 182, 184, 186, 188, 190; or 192, and/or comprises a light chain constant region comprising an amino acid sequence modified by 1-10 amino acid substitutions, 1-5 amino acid substitutions, or 1-3 amino acid substitutions compared to the amino acid sequence of SEQ ID NO: 194.
56. The isolated antibody of any one of embodiments 1-55, wherein the antibody comprises a human IgG1 or IgG2 heavy chain constant region comprising the amino acid sequence of any one of SEQ ID Nos: 174, 176, 178, 180, 182, 184, 186, 188, 190, or 192.
57. The isolated antibody of any one of embodiments 1-56, wherein the antibody comprises a light chain constant region comprising the amino acid sequence of SEQ ID NO: 194.
58. The isolated antibody of any one of embodiments 1-57, which is a full-length antibody. 59. The isolated antibody of any one of embodiments 1-57, which is an IgG antibody lacking a C-terminal lysine in the heavy chain constant region.
60. The isolated antibody of any one of embodiments 1-50, which is an antibody fragment, such as an Fv, single-chain Fv (scFv), Fab, Fab′, or (Fab′)₂.
61. An isolated antibody that specifically binds to protein arginine deiminase 4 (PAD4), wherein the antibody comprises a heavy chain (HC) comprising the amino acid sequence of SEQ ID NO: 68 followed by the amino acid sequence of SEQ ID NO: 178 and a light chain (LC) comprising the amino acid sequence of SEQ ID NO: 70 followed by the amino acid sequence of SEQ ID NO: 194.
62. An isolated antibody that specifically binds to protein arginine deiminase 4 (PAD4), wherein the antibody comprises a heavy chain (HC) comprising the amino acid sequence of SEQ ID NO: 68 followed by the amino acid sequence of SEQ ID NO: 180 and a light chain (LC) comprising the amino acid sequence of SEQ ID NO: 70 followed by the amino acid sequence of SEQ ID NO: 194.
63. An isolated antibody that specifically binds to protein arginine deiminase 4 (PAD4), wherein the antibody comprises an HC comprising the amino acid sequence of SEQ ID NO: 196 and a light chain comprising the amino acid sequence of SEQ ID NO: 200.
64. An isolated antibody that specifically binds to protein arginine deiminase 4 (PAD4), wherein the antibody comprises an HC comprising the amino acid sequence of SEQ ID NO: 198 and a light chain comprising the amino acid sequence of SEQ ID NO: 200.
65. The isolated antibody of any one of embodiments 1-64, which is a bispecific or multispecific antibody, or which is conjugated covalently or noncovalently to at least one other molecule.
66. The isolated antibody of embodiment 65, wherein the antibody is conjugated covalently or noncovalently to at least one other molecule, wherein the at least one other molecule comprises a detection label and/or a drug.
67. An isolated antibody that specifically binds to murine protein arginine deiminase 4 (PAD4), wherein the antibody comprises a heavy chain variable region (VH) comprising a heavy chain complementarity determining region 1 (HCDR1) comprising the amino acid sequence of positions 31-35 of SEQ ID NO: 208, an HCDR2 comprising the amino acid sequence of positions 50-66 of SEQ ID NO: 208, and an HCDR3 comprising the amino acid sequence of positions 99-107 of SEQ ID NO: 208; and wherein the antibody comprises a light chain variable region (VL) comprising a light chain complementarity determining region 1 (LCDR1) comprising the amino acid sequence of residues 24-38 of SEQ ID NO: 210, an LCDR2 comprising the amino acid sequence of residues 54-60 of SEQ ID NO: 210, and an LCDR3 comprising the amino acid sequence of residues 93-101 of SEQ ID NO: 210.
68. The isolated antibody of embodiment 67, wherein the antibody comprises a human IgG1 heavy chain variable region (VH) comprising an amino acid sequence at least 90%, at least 95%, at least 97%, or at least 99% identical to the amino acid sequence of SEQ ID No: 208; and/or wherein the antibody comprises a light chain variable region (VL) comprising an amino acid sequence at least 90%, at least 95%, at least 97%, or at least 99% identical to the amino acid sequence of SEQ ID NO: 210.
69. The isolated antibody of embodiment 67, wherein the antibody comprises a VH amino acid sequence modified by 1-10 amino acid substitutions, 1-5 amino acid substitutions, or 1-3 amino acid substitutions compared to the amino acid sequence of any one of SEQ ID Nos: 208; and/or wherein the antibody comprises a VL amino acid sequence modified by 1-10 amino acid substitutions, 1-5 amino acid substitutions, or 1-3 amino acid substitutions compared to the amino acid sequence of SEQ ID NO: 210.
70. The isolated antibody of any one of embodiments 67-69, wherein the antibody comprises a VH amino acid sequence of SEQ ID No: 208 and/or a VL amino acid sequence of SEQ ID NO: 210.
71. The isolated antibody of any one of embodiments 67-70, wherein the antibody comprises a heavy chain comprising an amino acid sequence at least 90%, at least 95%, at least 97%, or at least 99% identical to the amino acid sequence of SEQ ID No: 210 and/or wherein the antibody comprises a light chain comprising an amino acid sequence at least 90%, at least 95%, at least 97%, or at least 99% identical to the amino acid sequence of SEQ ID No: 212.
72. The isolated antibody of any one of embodiments 67-71, wherein the antibody comprises a heavy chain comprising the amino acid sequence of SEQ ID No: 212 and/or wherein the antibody comprises a light chain comprising the amino acid sequence of SEQ ID No: 214.
73. The isolated antibody of any one of embodiments 1-66, wherein the antibody does not bind to human protein arginine deiminase 2 (PAD2).
74. The isolated antibody of any one of embodiments 1-66 or 73, wherein the antibody inhibits PAD4 conversion of arginine in peptide substrate TSTGGRQGSHH (SEQ ID NO: 216) to citrulline with an IC50 of 10-200 nM, 50-200 nM, 10-100 nM, 20-100 nM, or 50-100 nM.
75. The isolated antibody of any one of embodiments 1-66, 73, or 74, wherein the antibody inhibits PAD4 conversion of arginine in peptide substrate (SHQESTRGKSKGKAAAAA; SEQ ID NO: 232) to citrulline with an IC50 of 0.1-10 nM, such as 0.2-5 nM, optionally in a dose-dependent manner with a PAD4 concentration of 1-8 μg/mL.
76. The isolated antibody of any one of embodiments 1-66 or 73-75, wherein the antibody inhibits PAD4 deamination of Na-benzoyl-L-arginine ethyl ester hydrochloride (BAEE).
77. The isolated antibody of any one of embodiments 1-66 or 73-76, wherein the antibody specifically binds to PAD4 with a K_{D} less than 5 nM, less than 3 nM, less than 1 nM, less than 0.5 nM, less than 0.1 nM, from 0.01 nM to 5 nM, from 0.01 nM to 1 nM, from 0.05 nM to 1 nM, from 0.1 nM to 1 nM, from 0.1 nM to 0.5 nM, from 0.05 nM to 0.5 nM, from 0.05 nM to 0.1 nM, or from 0.5 nM to 1 nM.
78. The isolated antibody of embodiment 77, wherein the antibody specifically binds to PAD4 with a K_{D} less than 5 nM, less than 3 nM, less than 1 nM, less than 0.5 nM, less than 0.1 nM, from 0.01 nM to 5 nM, from 0.01 nM to 1 nM, from 0.05 nM to 1 nM, from 0.1 nM to 1 nM, from 0.1 nM to 0.5 nM, from 0.05 nM to 0.5 nM, from 0.05 nM to 0.1 nM, or from 0.5 nM to 1 nM, as determined by surface plasmon resonance (SPR) (a) at 37° C. in the presence of 1-2 mM calcium chloride (e.g., 1 mM calcium chloride) and/or (b) at 37° C. in the absence of added Ca²⁺ and in the presence of 2 mM EDTA.
79. The isolated antibody of any one of embodiments 1-66 or 73-78, wherein the antibody has an ECM score of less than 50, less than 30, less than 10, less than 5, 1-30, 1-20, 1-10, 1-5, 1-3, 1, 2, 3, 4, or 5 in an ECM assay.
80. The isolated antibody of any one of embodiments 1-66 or 73-79, wherein the antibody is less immunogenic than one, two, or all three of Campath® (alemtuzumab), Rituxan® (rituximab), Zenapax® (daclizumab), as measured in an in silico immunogenicity assay.
81. The isolated antibody of any one of embodiments 1-66 or 73-80, wherein the antibody reduces the amount of extracellular citrullinated histone H3 and/or reduces secretion of GM-CSF and/or gene expression of GM-CSF in LPS-stimulated human blood monocytes compared to an isotype control antibody.
82. The isolated antibody of any one of embodiments 1-66 or 73-81, wherein the antibody is capable of being internalized by LPS-stimulated human blood monocytes.
83. The isolated antibody of any one of embodiments 1-82, wherein the antibody inhibits PAD4 function in an inflamed lung, for example, as demonstrated by decreased citrullination (e.g., of H3) in broncheoalveolar lavage fluid (BALF) collected from the lung.
84. The isolated antibody of any one of embodiments 1-83, wherein the antibody inhibits PAD4 function in an inflamed joint, for example, as demonstrated by decreased citrullination, e.g., of ITIH4 and/or PRG4 in joint tissue.
85. The isolated antibody of any one of embodiments 1-84, wherein the antibody reduces citrullination (e.g., of H3) in neutrophils, monocytes, M1 macrophages, and/or M2 macrophages compared to an isotype control antibody.
86. The isolated antibody of any one of embodiments 1-66 or 73-85, wherein the antibody reduces the amount of extracellular citrullination (e.g., of H3 and/or ITIH4) in BALF in an LPS-induced acute lung inflammation model with human PAD4 knock-in mice compared to an isotype control antibody, optionally wherein the amount of citrullinated H3 is reduced by at least 15%, at least 30%, at least 40%, at least 50%, or at least 60%, and/or wherein the amount of citrullinated ITIH4 is reduced by at least 50%, at least 70%, or at least 80%.
87. The isolated antibody of any one of embodiments 1-66 or 73-86, wherein the antibody reduces the amount of extracellular citrullination (e.g., of PRG4 and/or ITIH4) in mouse patella in an LPS-induced acute joint injury model with human PAD4 knock-in mice compared to an isotype control antibody, optionally wherein the EC50 of reduction of citrullinated ITIH4 and/or citrullinated PRG4 is 2 nM or lower, 1 nM or lower, 0.1 nM or lower, 0.05-2 nM, or 0.1-1 nM.
88. A pharmaceutical composition comprising an antibody of any one of embodiments 1-87 and a pharmaceutically acceptable carrier.
89. An isolated nucleic acid or set of two or more nucleic acids encoding the antibody of any of embodiments 1-87.
90. An isolated vector comprising one or more nucleic acids encoding the heavy chain and the light chain of the antibody of any one of embodiments 1-87.
91. An isolated host cell comprising the nucleic acid of embodiment 89 or the vector of embodiment 90.
92. A method of producing an antibody that specifically binds to PAD4 comprising culturing the host cell of embodiment 91 under conditions suitable for the expression of the antibody.
93. The method of embodiment 92, further comprising recovering the antibody from the host cell.
94. An antibody produced by the method of embodiment 92 or 93.
95. Use of the antibody of any one of embodiments 1-87 or the pharmaceutical composition of embodiment 88 in the preparation of a medicament for treating an autoimmune disorder.
96. The use of embodiment 95, wherein the autoimmune disorder is rheumatoid arthritis, lupus (e.g., systemic lupus erythematosus (SLE)), lupus nephritis, vasculitis (e.g., ANCA-associated vasculitis), thrombosis (e.g., venous thrombosis), inflammatory bowel disease (IBD) (e.g., ulcerative colitis, Crohn's disease), or another autoimmune disorder described herein.
97. The isolated antibody of any one of embodiments 1-87 or the pharmaceutical composition of embodiment 88 for use in treating an autoimmune disorder.
98. The antibody for use of embodiment 97, wherein the autoimmune disorder is rheumatoid arthritis, lupus (e.g., systemic lupus erythematosus (SLE)), lupus nephritis, vasculitis (e.g., ANCA-associated vasculitis), thrombosis (e.g., venous thrombosis), inflammatory bowel disease (IBD) (e.g., ulcerative colitis, Crohn's disease), or another autoimmune disorder described herein.
99. A method of treating an autoimmune disorder in a subject in need thereof, comprising administering to the subject an effective amount of the isolated antibody of any one of embodiments 1-87 or the pharmaceutical composition of embodiment 88.
100. The method of embodiment 99, wherein the autoimmune disorder is rheumatoid arthritis, lupus (e.g., systemic lupus erythematosus (SLE)), lupus nephritis, vasculitis (e.g., ANCA-associated vasculitis), thrombosis (e.g., venous thrombosis), inflammatory bowel disease (IBD) (e.g., ulcerative colitis, Crohn's disease), or another autoimmune disorder described herein.
101. The use, antibody for use, or method of any one of embodiments 95-100, wherein the method or use comprises administering at least one further therapeutic agent, optionally wherein the at least one further therapeutic agent is one or more of methotrexate, adalimumab, etanercept, infliximab, hydroxychloroquine, sulfasalazine, leflunomide, abatacept, anakinra, certolizumab, golimumab, rituximab, sarilumab, tocilizumab, baricitinib, tofacitinib, or upadacitinib.

Further exemplary embodiments include an antibody of any one of embodiments 1-87, comprising at least one post-translational modification of the amino acid sequence. In some embodiments, the modification is a modification of an N-terminal Glu or Gln to a pyroglutamate. In some such embodiments, a light chain N-terminal Glu or Gln is modified to pyroglutamate.

The disclosure herein also comprises use of an antibody of any one of the above embodiments 1-87 or the pharmaceutical composition of embodiment 88 described above in the preparation of a medicament for treating a subject at risk of developing RA, and an isolated antibody of any one of the above embodiments 1-87 or the pharmaceutical composition above of embodiment 88 for use in treating a subject at risk of developing RA. The disclosure also encompasses a method of treating a subject at risk of developing RA, comprising administering to the subject an effective amount of the isolated antibody of any one of the embodiments 1-87 above or the pharmaceutical composition of embodiment 88. In some cases, the subject has one or more of the following conditions: (a) at least one first-degree relative with RA (e.g. a parent or sibling); (b) presence of anti-citrullinated protein antibodies (ACPA) in serum; (c) presence of rheumatoid factor (RF) in serum; (d) arthralgia in at least one joint; (e) presence of inflammation in at least one joint observed by ultrasound or magnetic resonance imaging (MRI); or (f) undifferentiated arthritis. In some cases, the subject is in remission. In some cases, the method or use comprises administering at least one further therapeutic agent, optionally wherein the at least one further therapeutic agent is one or more of methotrexate, adalimumab, etanercept, infliximab, hydroxychloroquine, sulfasalazine, leflunomide, abatacept, anakinra, certolizumab, golimumab, rituximab, sarilumab, tocilizumab, baricitinib, tofacitinib, or upadacitinib.

In further embodiments, an antibody of any one of embodiments 1-87 or the pharmaceutical composition of embodiment 88 may be used in the preparation of a medicament for inhibiting NETosis or METosis in a subject. In some embodiments, the subject is a subject with an autoimmune disorder such as, for instance, rheumatoid arthritis, lupus (e.g., systemic lupus erythematosus (SLE)), lupus nephritis, vasculitis (e.g., ANCA-associated vasculitis), thrombosis (e.g., venous thrombosis), inflammatory bowel disease (IBD) (e.g., ulcerative colitis, Crohn's disease), or a subject at risk of developing an autoimmune disease, e.g., rheumatoid arthritis. Additional embodiments include an isolated antibody of any one of embodiments 1-87 or the pharmaceutical composition of claim 88 for use in inhibiting NETosis or METosis in a subject. In some embodiments, the subject is a subject with an autoimmune disorder, such as, for instance, rheumatoid arthritis, lupus, lupus nephritis, vasculitis, or thrombosis (e.g., venous thrombosis), inflammatory bowel disease (IBD) (e.g., ulcerative colitis, Crohn's disease) or another autoimmune disorder disclosed herein. In some embodiments, the subject is a subject at risk of developing an autoimmune disorder, e.g., rheumatoid arthritis. Further embodiments also include a method of inhibiting NETosis or METosis in a subject, the method comprising administering an effective amount of an antibody of any one of embodiments 1-87 or the pharmaceutical composition of embodiment 88 to the subject. In some embodiments, the subject is a subject with an autoimmune disorder such as, for instance, rheumatoid arthritis, lupus (e.g., systemic lupus erythematosus (SLE)), lupus nephritis, vasculitis (e.g., ANCA-associated vasculitis), thrombosis (e.g., venous thrombosis), inflammatory bowel disease (IBD) (e.g., ulcerative colitis, Crohn's disease), or another autoimmune disorder disclosed herein. In some embodiments, the subject is a subject at risk of developing an autoimmune disorder, e.g., rheumatoid arthritis. subject at risk of developing an autoimmune disease, e.g., rheumatoid arthritis. In some cases, the method or use comprises administering at least one further therapeutic agent, optionally wherein the at least one further therapeutic agent is one or more of methotrexate, adalimumab, etanercept, infliximab, hydroxychloroquine, sulfasalazine, leflunomide, abatacept, anakinra, certolizumab, golimumab, rituximab, sarilumab, tocilizumab, baricitinib, tofacitinib, or upadacitinib. The disclosure herein also encompasses an in vitro method of inhibiting NETosis or METosis in a biological sample, comprising administering an effective amount of the isolated antibody of any one of embodiments 1-87 or the pharmaceutical composition of embodiment 88 to the biological sample. Methods and uses herein also comprise methods of inhibiting citrullination in a subject, comprising administering to the subject an effective amount of the isolated antibody of any one of embodiments 1-87, or use of the isolated antibody of any one of embodiments 1-87 for inhibiting citrullination in a subject, or use of the isolated antibody of any one of embodiments 1-87 for preparation of a medicament for inhibiting citrullination in a subject. The disclosure also includes in vitro methods of inhibiting citrullination in a biological sample, comprising administering to the biological sample an effective amount of the isolated antibody of any one of embodiments 1-87.

In further embodiments, an antibody of any one of embodiments 1-87 or the pharmaceutical composition of embodiment 88 may be used in the preparation of a medicament for preventing onset or recurrence of an autoimmune disorder, such as rheumatoid arthritis, lupus (e.g., systemic lupus erythematosus (SLE)), lupus nephritis, vasculitis (e.g., ANCA-associated vasculitis), thrombosis (e.g., venous thrombosis), inflammatory bowel disease (IBD) (e.g., ulcerative colitis, Crohn's disease), or another autoimmune disorder disclosed herein, or an isolated antibody of any one of embodiments 1-87 or the pharmaceutical composition of claim 88 may be for use in preventing onset or recurrence of an autoimmune disorder, such as rheumatoid arthritis, lupus (e.g., systemic lupus erythematosus (SLE)), lupus nephritis, vasculitis (e.g., ANCA-associated vasculitis), thrombosis (e.g., venous thrombosis), inflammatory bowel disease (IBD) (e.g., ulcerative colitis, Crohn's disease), or another autoimmune disorder disclosed herein. Yet further embodiments include a method of preventing onset or recurrence of an autoimmune disorder, such as rheumatoid arthritis, lupus, lupus nephritis, vasculitis, thrombosis (e.g., venous thrombosis), inflammatory bowel disease (IBD), colitis, ulcerative colitis, or another autoimmune disorder disclosed herein, in a subject in need thereof, comprising administering an effective amount of the isolated antibody of any one of embodiments 1-87 or the pharmaceutical composition of claim 88 to the subject. In some embodiments, the subject has been determined to be susceptible to onset or recurrence of the disorder. In some such uses and methods herein, the antibody is administered to the subject along with an additional therapeutic agent.

In additional embodiments, the isolated antibody of any one of embodiments 1-87 or the pharmaceutical composition of embodiment 88 may be used in the preparation of a medicament for treating cancer, or the isolated antibody or pharmaceutical composition may be for use in treating cancer. Further embodiments also include a method of treating cancer in a subject in need thereof, comprising administering to the subject an effective amount of the isolated antibody of any one of embodiments 1-87 or the pharmaceutical composition of embodiment 88. In some cases, such a method or use comprises administering to the subject at least one further therapeutic agent, such as an immune checkpoint inhibitor, a chemotherapy agent, an anti-angiogenesis agent, or an anti-neoplastic agent, or a further therapy such as radiation therapy, hormonal therapy, or surgical treatment.

In further embodiments, an antibody of any one of embodiments 1-87 or the pharmaceutical composition of embodiment 88 may be used in the preparation of a medicament for treating an infectious disease, or an isolated antibody of any one of embodiments 1-87 or the pharmaceutical composition of claim 88 may be for use in treating an infectious disease. Yet further embodiments include a method of treating an infectious disease in a subject in need thereof, comprising administering an effective amount of an antibody herein. In some such uses and methods herein, the antibody is administered along with an additional therapeutic agent.

It is to be understood that both the foregoing general description and the following detailed description are exemplary and explanatory only and are not restrictive of the claims. All references cited herein are incorporated in their entirety by reference.

### BRIEF DESCRIPTION OF THE DRAWINGS

FIGS. 1A-1D show activities of exemplary murine anti-human PAD4 antibody (Ab) clones. FIG. 1A and FIG. 1B show effect of antibody clones on PAD4 activity, based on conversion of arginine to citrulline, with FIG. 1A showing percent PAD4 activity and FIG. 1B showing citrulline concentration in the presence of the antibody clones. FIG. 1C and FIG. 1D show that antibodies inhibit PAD4 in a dose-dependent manner. FIG. 1C shows a dose-response curve for antibody clone 13. FIG. 1D shows a dose-response curve for antibody clone 20.

FIGS. 1E-1H show the VH and VL amino acid sequences of clones 13 (VH=FIG. 1E; VL=FIG. 1F) and 20 (VH=FIG. 1G; VL=FIG. 1H). In the sequences shown in FIGS. 1E-1H, (1) the HCDR1, HCDR2, and HCDR3 sequences are located at amino acid positions 26-35, 50-65, and 95-102, respectively; and (2) the LCDR1, LCDR2, and LCDR3 sequences are located at amino acid positions 24-34, 50-56, and 89-97, respectively. In FIGS. 1E and 1G, HCDR1 at is shown at amino acid positions 26-35 (FIGS. 1E and 1G) according to the AbM definition of CDRs (Abhinandan and Martin, Mol. Immunol. 45:3832-3839 (2008); Swindells et al., J. Mol. Biol. 429:356-364 (2017)). According to the Kabat numbering scheme, HCDR1 is at amino acid positions 31-35 instead (not shown). The remaining CDRs (HCDR2, HCDR3, LCDR1, LCDR2, and LCDR3; FIGS. 1E-1H) are numbered according to the Kabat numbering scheme.

FIG. 2 shows that the PAD4 antibodies are specific for PAD4 and do not substantially inhibit the activity of PAD2.

FIGS. 3A-3X show antibody stability as assessed by size exclusion chromatography (SEC) and peptide mapping. FIG. 3A shows SEC results for 20 exemplary humanized (hz) clone 13 and 20 antibodies. FIG. 3B shows peptide mapping results for exemplary hz clone 20 antibodies. FIGS. 3C-3D show peptide mapping results for exemplary hz clone 13 antibodies. FIG. 3E and FIG. 3F show SEC results for hz13-12 and hz13-5, respectively. FIGS. 3G-3J show peptide mapping results for hz13-12. FIGS. 3K-3N show peptide mapping results for hz13-5. FIG. 3O and FIG. 3P show SEC results for hz13-12 D31E and hz13-5 D31E, respectively. FIG. 3Q and FIG. 3R show peptide mapping results for hz13-12 D31E and hz13-5 D31E, respectively. FIG. 3S shows SEC results for hz13-5. FIG. 3T shows SEC results for hz13-5 D31E. FIG. 3U and FIG. 3V show peptide mapping results for high concentration hz13-5 and hz13-5D31E, respectively. FIG. 3W and FIG. 3X show SEC results and peptide mapping results, respectively, for hz13-5 D31E.

FIGS. 4A-4B show epitopes determined for clone 13 (FIG. 4A) and clone 20 (FIG. 4B) by HDX-MS. Regions in hPAD4 with significant HDX reduction upon Fab binding are boxed (black lined box for primary epitope; gray lined box for secondary epitope). For each antibody, the primary epitope is shown in underlined black text above the corresponding black lined box, and the secondary epitope is shown in smaller text above the corresponding gray lined box.

FIGS. 5A-5B show binding residues determined for clone 13 (FIG. 5A) and clone 20 (FIG. 5B) by FPOP, GEE, and DEPC labeling. The protected residues, with statistical significance of the difference in % labeling between hPAD4 and hPAD4/Fab (based on student T-test p value <0.01), are marked with star. Insufficient MS/MS fragments were obtained to differentiate the protection on H644 and C645; both of these residues were considered binding residues for clone 20.

FIG. 6 shows an epitope summary for clone 13 and clone 20. The epitope regions determined by HDX-MS are boxed. The binding residues determined by FPOP, GEE, and DEPC labeling are underlined and highlighted in bold.

FIGS. 7A-7B show sequence coverage by pepsin for the clone 13 heavy chain (HC; FIG. 7A) and the clone 13 light chain (LC; FIG. 7B). Each bar indicates a pepsin peptide. Complimentary Determining Regions (CDRs) are boxed.

FIGS. 8A-8B show regions with significant HDX reduction in the clone 13 Fab HC (FIG. 8A), and the clone 13 Fab LC (FIG. 8B) upon binding with hPAD4. Paratope regions for clone 13 are boxed.

FIGS. 9A-9B show the cryo-electron microscopy (Cryo-EM) structure of the PAD4.clone 13 Fab complex. FIG. 9A shows a PAD4.clone 13 Fab monomer. PAD4 residues are numbered in three letter code. The approximate position of the catalytic site is circled. FIG. 9B shows a PAD4.Fab13 dimer.

FIG. 10 shows residue D31 of clone 13 and interactions at the interface of PAD4 and the heavy chain of the clone 13 Fab. Fab residues are numbered in the single residue code whereas PAD4 residues are numbered in three-letter code.

FIG. 11 shows the substrate binding site of human PAD4 (PDB Code 2DEW). The lysine loop and the catalytic coil are shown (the lysine loop includes LYS-519 and ILE-526 and the catalytic coil includes ILE-630 and ILE-638). The arginine residue of the co-crystallized substrate peptide is shown in stick representation. Ca²⁺ ions from 2DEW structure are shown as spheres.

FIG. 12 shows superposition of PAD4 chains from 2DEW and PAD.clone 13 Fab cryoEM structures. 2DEW is shown in cartoon representation in light gray shade whereas PAD4 from PAD4.clone 13 Fab cryoEM structure is shown as black cartoon. Heavy and Light chains of clone 13 Fab are shown as thin ribbons. PAD4 residues are numbered in three letter code whereas clone 13 Fab residues are numbered in single letter code. Movement of the lysine loop and its impact on the catalytic coil has been shown by black arrows. Ca²⁺ ions from 2DEW structure are shown as spheres.

FIGS. 13A-13N show heatmaps (FIGS. 13A-13L) and SPR (FIGS. 13M-13N) measurements from the studies of pH dependent anti-PAD4 antibodies. In the heatmaps shown in FIGS. 13A-13L, (1) ER values less than 1 indicated that the substitution would weaken binding to PAD4, (2) numbers greater than 1.00 indicate an amino acid substitution would result in stronger binding to PAD4, and (3) 1.00 indicates an amino acid substitution that would not result in a change in binding (i.e., binding was not stronger or weaker because of the mutation). The pH dependent anti-PAD4 antibodies were tested at pH 7 (FIGS. 13A, 13C, 13E, 13G, 13I, and 13K) and pH 6 (FIGS. 13B, 13D, 13F, 13H, 13J, and 13L). FIG. 13A shows HCDR1 of clone 13 (mAb13) at pH 7. FIG. 13B shows HCDR1 of clone 13 (mAb13) at pH 6. FIG. 13C shows HCDR2 of clone 13 (mAb13) at pH 7. FIG. 13D shows HCDR2 of clone 13 (mAb13) at pH 6. FIG. 13E shows HCDR3 of clone 13 (mAb13) at pH 7. FIG. 13F shows HCDR3 of clone 13 (mAb13) at pH 6. FIG. 13G shows LCDR1 of clone 13 (mAb13) at pH 7. FIG. 13H shows LCDR1 of clone 13 (mAb13) at pH 6. FIG. 13I shows LCDR2 of clone 13 (mAb13) at pH 7. FIG. 13J shows LCDR2 of clone 13 (mAb13) at pH 6. FIG. 13K shows HCDR3 of clone 13 (mAb13) at pH 7. FIG. 13L shows LCDR3 of clone 13 (mAb13) at pH 6. FIGS. 13M-13N show SPR measurements recorded at pH 7.6 (FIG. 13M) and pH 6.0 (FIG. 13N) of an exemplary anti-PAD4 antibody hz13-5. The pH-dependent variant* is hz13-5 VH_D31H::Vk_I30H. Slightly increasing signal during the dissociation phase was observed at pH 6.0 likely due to lower stability of PAD4 at pH 6.0 which may lead to some aggregation of PAD4 on the surface.

FIG. 14 shows percentage of healthy volunteer human donors (n=40) with a positive CD4+ proliferation response after seven days of incubation of autologous antigen-pulsed monocyte-derived DCs with antibodies Avastin, IL-21R mAb (ATR-107), clone 13-based hz13-12 antibody, clone 20-based hz20-7 clone 13-based hz13-5, and clone 13-based hz13-5 D31E.

FIG. 15 shows dose-dependent inhibition of human PAD4 with antibody hz13-5 D31E at four concentrations of recombinant human PAD4 (rhPAD4) ranging from 13.5 nM (1 μg/mL) to 108 nM (8 μg/mL).

FIGS. 16A-16F show reduced extracellular citrullinated histone 3 in lipopolysaccharide (LPS)-stimulated human CD14+ monocytes treated with the specified concentrations of indicated clone 13 (C13) or clone 20 (C20) antibody variant or with isotype control antibody. FIG. 16A shows citrullinated histone 3 levels in monocytes incubated with hz13-5. FIG. 16B shows citrullinated histone 3 levels in monocytes incubated with hz13-5 D31E. FIG. 16C citrullinated histone 3 levels in monocytes incubated with hz20-2. FIG. 16D shows citrullinated histone 3 levels in monocytes incubated with hz20-7. FIG. 16E shows citrullinated histone 3 levels in monocytes incubated with hz13-3. FIG. 16F shows citrullinated histone 3 levels in monocytes incubated with hz13-12.

FIGS. 17A-17F show reduced secretion of GM-CSF (ng/mL) in LPS-stimulated human CD14+ monocytes treated with the specified concentrations of indicated clone 13 (C13) or clone 20 (C20) antibody variant or with isotype control antibody. FIG. 17A shows detection of GM-CSF in monocytes incubated with hz13-5. FIG. 17B shows GM-CSF levels in monocytes incubated with hz13-5 D31E. FIG. 17C shows GM-CSF levels in monocytes incubated with hz13-3. FIG. 17D shows GM-CSF levels in monocytes incubated with hz13-12. FIG. 17E shows GM-CSF levels in monocytes incubated with hz20-2. FIG. 17F shows GM-CSF levels in monocytes incubated with hz20-7.

FIGS. 18A-18B show reduced GM-CSF gene expression (fold induction, calculated based on 2,-deltadeltaCT)) in LPS-stimulated human CD14+ monocytes treated with the specified concentrations of indicated clone 13 (C13) antibody variant. FIG. 18A shows fold induction of GM-CSF in monocytes incubated with hz13-5. FIG. 18B shows fold induction of GM-CSF in monocytes incubated with hz13-5 D31E. GM-CSF gene Ct values were normalized to housekeeping gene the Peptidyl-prolyl cis-trans isomerase A (PPIA) by subtracting from PPIA Ct values to generate DCts. The relative quantity (RQ) value (fold induction) for each sample to the housekeepers was calculated using the following formula: RQ=2^{-DCt}.

FIGS. 19A-19B shows detections of the anti-PAD4 antibody hz13-5 D31E in acidic cellular compartments. FIG. 19A shows images of donor GAC-077 of Fabfluor-pH red staining (left panels) and overlay of FabFluor-pH with Cytotox Green staining (middle and right panels; co-staining appears yellow) at each indicated conditions (middle panels=50 μg/mL hz13-5 D31E; right panels=50 μg/mL isotype control) at 48 h. FIG. 19B shows antibody internalization area (μm²/image) measured using FAbFluor-pH staining across conditions for the 2 donors GAC-077 and GAC-045.

FIG. 20 shows in vivo efficacy of an exemplary anti-PAD4 antibody, the anti-murine PAD4 mAb, mumAb, in a lipopolysaccharide (LPS) induced acute lung inflammation pharmacodynamics (ALI PD) model. The 30 mpk and 100 mpk treatments respectively resulted in 74% and 83% inhibition of extracellular citrullinated H3 compared to the isotype control (100 mpk) condition. “Mpk”=mg/kg. “IC”=Isotype Control antibody. “Naïve”=not induced with LPS. Percent inhibition was calculated as =[(IC−naïve)−(PAD4 mAb-naïve)/(IC−naïve)]×100.

FIGS. 21A-21B show experimental design of acute joint inflammation (AJI) and chronic joint inflammation (CJI) studies. FIG. 21A shows the timetables for administration of treatment (subcutaneous anti-PAD4 antibody or isotype control), administration of LPS to induce inflammation, and assessment of outcomes. FIG. 21B shows procedures for induction and assessment of outcomes.

FIGS. 22A-22B show reduction of extracellular citrullinated proteins by the anti-murine PAD4 mAb, mumAb, in an LPS induced acute joint inflammation (AJI) pharmacodynamics (PD) model. FIG. 22A shows the Cit-ITIH4 results. FIG. 22B shows the Cit-PRG4 results. Percent inhibition was calculated in an analogous manner as described for FIG. 20. “Mpk”=mg/kg. “Isotype”=Isotype Control antibody (IC). “Naïve”=not injected with LPS.

FIGS. 23A-23B show reduction in extracellular citrullinated proteins by the anti-murine PAD4 mAb, mumAb, in an LPS induced chronic joint inflammation (CJI) pharmacodynamics (PD) model. FIG. 23A shows the Cit-ITIH4 results. FIG. 23B shows the Cit-PRG4 results. Percent inhibition was calculated in an analogous manner as described for FIG. 20. “Mpk”=mg/kg. “Isotype”=Isotype Control antibody (IC). “Naïve”=not injected with LPS. “PBS”=naïve mice sham injected with phosphate buffered saline (PBS) in place of LPS.

FIGS. 24A-24B show reduction of NETosis (FIG. 24A) and METosis (FIG. 24B) by the anti-murine PAD4 mAb anti-PAD4 mAb, mumAb, in a pristane-induced peritonitis model. N=6 per treatment group. *p<0.05, **p<0.01, ***p<0.001 versus isotype control by one-way ANOVA with a Dunnett test. “Mpk”=mg/kg. “ISO”=Isotype Control antibody (IC). “Naïve”=sham injected with PBS in place of LPS.

FIGS. 25A-25B show reduction of Cit-H3 in neutrophils and monocytes by anti-murine PAD4 mAb, mumAb, in the pristane-induced peritonitis model. FIG. 25A shows % cells and FIG. 25B shows cell numbers. N=6 per treatment group. *p<0.05, **p<0.01, ***p<0.001 versus isotype control by one-way ANOVA with a Dunnett test. “Mpk”=mg/kg. “ISO”=Isotype Control antibody (IC). “Naïve”=did not receive pristane injection.

FIGS. 26A-26B show reduction of Cit-H3 in M1 macrophages (FIG. 26A) and M2 macrophages (FIG. 26B) by anti-murine PAD4 mAb, mumAb, in the pristane-induced peritonitis model. N=6 per treatment group. *p<0.05, **p<0.01, ***p<0.001 versus isotype control by one-way ANOVA with a Dunnett test. “Mpk”=mg/kg. “ISO”=Isotype Control antibody (IC). “Naïve”=not induced with pristane.

FIGS. 27A-27B show reduction in elastase (FIG. 27A) and MPO (FIG. 27B) in peritoneal fluid samples by anti-murine PAD4 mAb, mumAb, in the pristane-induced peritonitis model. N=6 per treatment group. *p<0.05, **p<0.01, ***p<0.001, ****p<0.0001 versus isotype control by one-way ANOVA with a Dunnett test. “Mpk”=mg/kg. “ISO”=Isotype Control antibody (IC). “Naïve”=not induced with pristane.

FIGS. 28A-28B show reduction in chemokines and cytokines by anti-murine PAD4 mAb, mumAb, in the pristane-induced peritonitis model. FIG. 28A shows GRO beta/MIP-2 alpha, GRO alpha/KC, MCP 1, and MIP 1beta levels in peritoneal fluid. FIG. 28B shows IL-6 and MIP 3a levels in plasma. N=6 per treatment group. *p<0.05, **p<0.01, ***p<0.001, ****p<0.0001 versus isotype control by one-way ANOVA with a Dunnett test. “Mpk”=mg/kg. “ISO”=Isotype Control antibody (IC). “Naïve”=not induced with pristane.

FIGS. 29A-29C show results from a preventative study in a collagen-induced arthritis (CIA) model. Anti-murine PAD4 mAb, mumAb, reduced clinical score (FIG. 29A), disease incidences (FIG. 29B), and paw weight (FIG. 29C). N=10-15 per treatment group. *p<0.05, **p<0.01, ***p<0.001 versus IC by one-way ANOVA with a Dunnett test. “IC”=Isotype Control antibody. “Naïve”=not induced with collagen.

FIGS. 30A-30E show results from a preventative study in a CIA model. Anti-murine PAD4 mAb, mumAb, reduced immune cell infiltration in the paw. FIG. 30A: CD45+ cells. FIG. 30B: total myeloid cells; CD11b+ cells. FIG. 30C: neutrophils; CD11b+ Ly6G+ Ly6C− cells. FIG. 30D: monocytes; CD11b+ Ly6G− Ly6C+ cells. FIG. 30E: macrophages; F4/80+ cells. N=10-15 per treatment group. N=5 in naïve group. *p<0.05, **p<0.01, ***p<0.001 versus IC by one-way ANOVA with a Dunnett test. “Mpk”=mg/kg. “ISO”=Isotype Control antibody (IC). “Naïve”=not induced with collagen.

FIGS. 31A-31B show results from a preventative study in a CIA model. Anti-murine PAD4 mAb, mumAb, reduced NETosis (FIG. 31A) and METosis (FIG. 31B, in which the left graph shows number of monocytes and the right graph shows numbers of macrophages) in mouse paw. N=10-15 per treatment group. N=5 in naïve group. *p<0.05, **p<0.01, ***p<0.001 versus IC by one-way ANOVA with a Dunnett test. “Mpk”=mg/kg. “ISO”=Isotype Control antibody (IC). “Naïve”=not induced with collagen.

FIGS. 32A-32C show results from a preventative study in a CIA model. Anti-murine PAD4 mAb, mumAb, reduced total Cit-H3 in neutrophils (FIG. 32A), monocytes (FIG. 32B), and M2 macrophages (FIG. 32C) in mouse paw. N=10-15 per treatment group. N=5 in naïve group. *p<0.05, **p<0.01, ***p<0.001 versus IC by one-way ANOVA with a Dunnett test. “Mpk”=mg/kg. “ISO”=Isotype Control antibody (IC).

FIGS. 33A-33C show results from a preventative study in a CIA model. Anti-murine PAD4 mAb, mumAb, reduced PAD4 (FIG. 33A), MPO (FIG. 33B), and neutrophil elastase (FIG. 33C) in mouse paw. N=10-15 per treatment group. N=5 in naïve group. *p<0.05, **p<0.01, ***p<0.001 versus vehicle by one-way ANOVA with a Dunnett test. “Mpk”=mg/kg. “Naïve”=not induced with collagen.

FIGS. 34A-FIG. 34B show results from a preventative study in a CIA model. Anti-murine PAD4 mAb, mumAb, reduced anti-citrullinated peptide antibody (ACPA; FIG. 34A) in paw and serum and also reduced serum anti-CII antibody titer (FIG. 34B). N=10-15 per treatment group. N=5 in naïve group. *p<0.05, **p<0.01, ***p<0.001 versus IC by one-way ANOVA with a Dunnett test. “Mpk”=mg/kg. “Naïve”=not induced with collagen.

FIG. 35 shows results from a preventative study in a CIA model. Anti-murine PAD4 mAb, mumAb, reduced paw histology score (% reductions provided are relative to IC). N=10-15 per treatment group. *p<0.05, **p<0.01, ***p<0.001 versus vehicle by one-way ANOVA with a Dunnett test. “Mpk”=mg/kg. “IC”=Isotype Control antibody.

FIGS. 36A-36C show results from a semi-therapeutic study in a CIA model. Anti-murine PAD4 mAb, mumAb, reduced clinical score (FIG. 36A), disease incidences (FIG. 36B), and paw weight (FIG. 36C). N=10-15 per treatment group. *p<0.05, **p<0.01, ***p<0.001 versus IC by one-way ANOVA with a Dunnett test. “IC”=Isotype Control antibody.

FIGS. 37A-37E show results from a semi-therapeutic study in a CIA model. Anti-murine PAD4 mAb, mumAb, reduced immune cell infiltration in the paw. FIG. 37A: total cell infiltration; CD45+ cells. FIG. 37B: total myeloid cells; CD11b+ cells. FIG. 37C: neutrophils; CD11b+ Ly6G+ Ly6C− cells. FIG. 37D: monocytes; Ly6C+ cells. FIG. 37E: macrophages; F4/80+ cells. N=10-15 per treatment group. N=5 in naïve group. *p<0.05, **p<0.01, ***p<0.001 versus IC by one-way ANOVA with a Dunnett test. “Mpk”=mg/kg. “ISO”=Isotype Control antibody (IC). “Naïve”=not induced with collagen.

FIGS. 38A-38B show results from a semi-therapeutic study in a CIA model. Anti-murine PAD4 mAb, mumAb, reduced NETosis (FIG. 38A) and METosis (FIG. 38B) in mouse paw. N=10-15 per treatment group. N=5 in naïve group. *p<0.05, **p<0.01, ***p<0.001 versus IC by one-way ANOVA with a Dunnett test. “Mpk”=mg/kg. “ISO”=Isotype Control antibody (IC). “Naïve”=not induced with collagen.

FIGS. 39A-39C show results from a semi-therapeutic study in a CIA model. Anti-murine PAD4 mAb, mumAb, reduced total Cit-H3 in neutrophils (FIG. 39A), monocytes (FIG. 39B), and M2 macrophages (FIG. 39C) in mouse paw. N=10-15 per treatment group. N=5 in naïve group. *p<0.05, **p<0.01, ***p<0.001 versus vehicle by one-way ANOVA with a Dunnett test. “Mpk”=mg/kg. “ISO”=Isotype Control antibody (IC). “Naïve”=not induced with collagen.

FIGS. 40A-40C show results from a semi-therapeutic study in a CIA model. Anti-murine PAD4 mAb, mumAb, reduced PAD4 (FIG. 40A), MPO (FIG. 40B), and neutrophil elastase (FIG. 40C) in mouse paw. N=10-15 per treatment group. N=5 in naïve group. *p<0.05, **p<0.01, ***p<0.001 versus vehicle by one-way ANOVA with a Dunnett test. “Mpk”=mg/kg. “Naïve”=not induced with collagen.

FIGS. 41A-41B show results from a semi-therapeutic study in a CIA model. Anti-murine PAD4 mAb, mumAb, reduced ACPA in paw and serum (FIG. 41A, right and left panels respectively) and serum anti-CII antibody titer (FIG. 41B). N=10-15 per treatment group. N=5 in naïve group. *p<0.05, **p<0.01, ***p<0.001 versus vehicle by one-way ANOVA with a Dunnett test. “Mpk”=mg/kg.

FIG. 42 show results from a semi-therapeutic study in a CIA model. Anti-murine PAD4 mAb, mumAb, reduced paw histology score. N=10-15 per treatment group. *p<0.05, **p<0.01, ***p<0.001 versus vehicle by one-way ANOVA with a Dunnett test. “Mpk”=mg/kg. “IC”=Isotype Control antibody.

FIGS. 43A-43E show efficacy of anti-human PAD4 mAbs clone 20 and humanized derivatives in an LPS ALI PD model. FIG. 43A, FIG. 43B, and FIG. 43C respectively show the effects of clone 20, humanized clone 20-based hz20-2 antibody and humanized clone 20-based hz20-7 antibody at the indicated doses on extracellular citrullinated H3 relative to total extracellular H3; the percent reductions indicate reductions relative to the isotype control condition. FIG. 43D and FIG. 43E respectively show the effects of clone 20-based hz20-2 antibody and clone 20-based hz20-7 antibody at the indicated doses on extracellular citrullinated ITIH4 relative to total extracellular ITIH4; the percent reductions indicate reductions relative to the isotype control condition. “Mpk”=mg/kg. “IC”=Isotype Control antibody. “Naïve”=not nebulized with LPS.

FIGS. 44A-44E show efficacy of anti-human PAD4 mAbs clone 13 and humanized derivatives in an LPS ALI PD model. FIG. 44A, FIG. 44B, and FIG. 44C respectively show the effects of clone 13, humanized clone 13-based hz13-12 antibody, and humanized clone 13-based hz13-5 antibody at the indicated doses on extracellular citrullinated H3 relative to total extracellular H3; the percent reductions indicate reductions relative to the isotype control condition. FIG. 44D and FIG. 44E respectively show the effects of clone 13-based hz13-12 antibody and clone 13-based hz13-5 antibody at the indicated doses on extracellular citrullinated ITIH4 relative to total extracellular ITIH4; the percent reductions indicate reductions relative to the isotype control condition. “Mpk”=mg/kg. “IC”=Isotype Control antibody. “Naïve”=not nebulized with LPS.

FIGS. 45A-45E show experimental design and human PAD4 endpoints of an LPS AJI PD study using Hu-PAD4 knock-in mice. FIG. 45A shows a study timetable. FIG. 45B shows an overview of experimental procedures including treatment at the knee joint and extraction of citrullinated proteins from explanted tissue. Extracellular Cit-PRG4 (FIG. 45C), extracellular Cit-ITIH4 (FIG. 45D) and human PAD4 (FIG. 45E) of patella explant supernatant are shown.

FIGS. 46A-46C show efficacy of anti-human PAD4 antibodies in an LPS AJI PD study. The effects of clone 13-based hz13-12 antibody (FIG. 46A), clone 13-based hz13-5 antibody (FIG. 46B), and clone 20-based hz20-2 antibody (FIG. 46C) at the indicated doses on extracellular citrullinated ITIH4 relative to total extracellular ITIH4 are shown; the percent reductions indicate reductions relative to the isotype control condition. “Mpk”=mg/kg. “IC”=Isotype Control antibody. “Naïve”=not injected with pristane. “PBS”=naïve, injected with phosphate buffered saline in place of LPS.

FIGS. 47A-47C show efficacy of anti-human PAD4 antibodies in an LPS AJI PD study. Extracellular Cit-PRG4 of patella explant supernatant is shown. The effects of clone 13-based hz13-12 antibody (FIG. 47A), clone 13-based hz13-5 antibody (FIG. 47B), and clone 20-based hz20-2 antibody (FIG. 47C) at the indicated doses on extracellular citrullinated PRG4 relative to total extracellular PRG4 are shown; the percent reductions indicate reductions relative to the isotype control condition. “Mpk”=mg/kg. “IC”=Isotype Control antibody. “PBS”=phosphate buffered saline. “Naïve”=not injected with LPS.

FIGS. 48A-48B show efficacy of anti-human PAD4 antibodies hz13-5 and hz13-5 D31E in an LPS AJI study. These antibodies inhibited citrullination of ITIH4 (FIG. 48A) and PRG4 (FIG. 48B); the results showed dose dependent inhibition by the hz13-5 D31E antibody. “Mpk”=mg/kg. “IC”=Isotype Control antibody. “Naïve”=not injected with LPS.

FIGS. 49A-49B show that the hz13-5 D31E antibody maintained potency in the presence of endogenous PAD4 antibodies from RA patients. FIG. 49A shows PAD4 autoantibodies measured by OD450 in ELISA from purified IgG of serum samples of 21 RA patients and 10 healthy control subjects (NHV). FIG. 49B shows inhibition of H3 citrullination by hz13-5 D31E in the presence of purified IgG from RA (closed squares) or NHV (open squares) serum. Dotted lines represent H3 citrullination by PAD4 in the presence of purified IgG from RA donors (lower line) and NHV donors (upper line) without hz13-5 D31E.

### DETAILED DESCRIPTION OF CERTAIN EMBODIMENTS

### I. Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

Exemplary techniques used in connection with recombinant DNA, oligonucleotide synthesis, tissue culture and transformation (e.g., electroporation, lipofection), enzymatic reactions, and purification techniques are described, e.g., in Sambrook et al. Molecular Cloning: A Laboratory Manual (2nd ed., Cold Spring Harbor Laboratory Press, Cold Spring Harbor, N.Y. (1989)), among other places.

As used herein, the term “about” refers to a numeric value, including, for example, whole numbers, fractions, and percentages, whether or not explicitly indicated. The term “about” generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

The term “polypeptide” refers to a polymer of amino acid residues, and is not limited to a minimum length. A “protein” may comprise one or more polypeptides. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” or “protein” refers to a polypeptide or protein, respectively, which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts that produce the proteins or errors due to PCR amplification. A protein may comprise two or more polypeptides.

“PAD4” or “protein arginine deiminase 4” or “peptidyl arginine deiminase 4,” as used herein, refers to human PAD4 (huPAD4; UniProt ID: Q9UM07), unless expressly noted otherwise (i.e., murine PAD4, cynomolgus PAD4, or the like). Exemplary human PAD4 amino acid sequences are shown in SEQ ID NO: 1 and SEQ ID NO: 2 and SEQ ID NO: 3.

The term “antibody” herein refers to a molecule comprising at least complementarity-determining region (CDR) 1, CDR2, and CDR3 of a heavy chain and at least CDR1, CDR2, and CDR3 of a light chain, wherein the molecule is capable of binding to antigen. The term is used in the broadest sense and encompasses various antibody structures, including but not limited to monoclonal antibodies, polyclonal antibodies, multi specific antibodies (e.g., bispecific antibodies, diabodies, etc.), full length antibodies, single-chain antibodies, antibody conjugates, and antibody fragments, so long as they exhibit the desired PAD4-specific binding activity.

An “isolated” antibody is one that has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

An “antigen” refers to the target of an antibody, i.e., the molecule to which the antibody specifically binds. The term “epitope” denotes the site on an antigen, either proteinaceous or non-proteinaceous, to which an antibody binds. Epitopes on a protein can be formed both from contiguous amino acid stretches (linear epitope) or comprise non-contiguous amino acids (conformational epitope), e.g., coming in spatial proximity due to the folding of the antigen, i.e., by the tertiary folding of a proteinaceous antigen. Linear epitopes are typically still bound by an antibody after exposure of the proteinaceous antigen to denaturing agents, whereas conformational epitopes are typically destroyed upon treatment with denaturing agents.

An “anti-PAD4 antibody” or a “PAD4-antibody” or an “antibody that specifically binds to PAD4” or an “antibody that binds to PAD4” and similar phrases refer to an antibody that specifically binds to PAD4 as defined herein.

The term “heavy chain” refers to a polypeptide comprising at least a heavy chain variable region, with or without a leader sequence. In some embodiments, a heavy chain comprises at least a portion of a heavy chain constant region. The term “full-length heavy chain” refers to a polypeptide comprising a heavy chain variable region and a heavy chain constant region, with or without a leader sequence.

The term “light chain” refers to a polypeptide comprising at least a light chain variable region, with or without a leader sequence. In some embodiments, a light chain comprises at least a portion of a light chain constant region. The term “full-length light chain” refers to a polypeptide comprising a light chain variable region and a light chain constant region, with or without a leader sequence.

The term “complementarity determining regions” (“CDRs”) as used herein refers to each of the regions of an antibody variable region which are hypervariable in sequence and which determine antigen binding specificity. Generally, antibodies comprise six CDRs: three in the VH (CDR-H1 or heavy chain CDR1, CDR-H2, CDR-H3), and three in the VL (CDR-L1, CDR-L2, CDR-L3). Unless otherwise indicated, the CDRs are determined according to the sequence table herein.

“Framework” or “FR” refers to the residues of the variable region residues that are not part of the complementary determining regions (CDRs). The FR of a variable region generally consists of four FRs: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). See, e.g., Kindt et al. Kuby Immunology, 6th ed., W.H. Freeman and Co., page 91 (2007). A variable domain may comprise heavy chain (HC) CDR1-FR2-CDR2-FR3-CDR3 with or without all or a portion of FR1 and/or FR4; and light chain (LC) CDR1-FR2-CDR2-FR3-CDR3 with or without all or a portion of FR1 and/or FR4. That is, a variable domain may lack a portion of FR1 and/or FR4 so long as it retains antigen-binding activity. A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The light chain and heavy chain “constant regions” of an antibody refer to additional sequence portions outside of the FRs and CDRs and variable regions. Certain antibody fragments may lack all or some of the constant regions. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain at Gly446 and Lys447 (EU numbering). Antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine and lysine, respectively. Therefore, the C-terminal lysine, or the C-terminal glycine and lysine, of the Fc region may or may not be present. Thus, a “full-length heavy chain constant region” or a “full length antibody” for example, which is a human IgG1 antibody, includes an IgG1 with both a C-terminal glycine and lysine, without the C-terminal lysine, or without both the C-terminal glycine and lysine. Unless otherwise specified herein, numbering of amino acid residues in the Fc region or constant region is according to the EU numbering system, also called the EU index, as described in Kabat et al., Sequences of Proteins of Immunological Interest, 5^{th} Ed. Public Health Service, National Institutes of Health, Bethesda, MD, 1991.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen (i.e., PAD4) to which the intact antibody binds. Examples of antibody fragments include but are not limited to Fv, Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The terms “full length antibody”, “intact antibody”, and “whole antibody” are used herein interchangeably to refer to an antibody having a structure substantially similar to a native antibody structure or, in the case of an IgG antibody, having heavy chains that contain an Fc region as defined herein above.

The term “chimeric” antibody refers to an antibody in which a portion of the heavy and/or light chain is derived from a particular source or species, while the remainder of the heavy and/or light chain is derived from a different source or species.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method.

A “multispecific” antibody is one that binds specifically to more than one target antigen, while a “bispecific” antibody is one that binds specifically to two antigens. An “antibody conjugate” is an antibody conjugated to one or more heterologous molecule(s), including but not limited to a therapeutic agent or a label.

Antibodies may be modified as part of the production process in certain host cells or through metabolism in vivo. An antibody or antibody region amino acid sequence herein is intended to encompass not only the specific amino acid sequence, but also that sequence as post-translationally modified, for instance, including side chain modifications and cleavages. Such a post-translational modification can occur, for instance, as a result of production of the antibody in a host cell and/or as a result of post-translational modification in vivo in an animal (e.g., a human).

In some embodiments, an antibody disclosed herein comprises a post-translational modification (e.g., one or more post-translational modifications). Post-translational modifications can include, e.g., ubiquitination, phosphorylation, acetylation, hydroxylation, methylation, glycyosylation, AMPylation, prenylation, deamidation, elimylation, citrullination, and carbamoylation. In some embodiments, the antibody is not post-translationally modified.

As noted above, antibodies can undergo post-translational cleavage of one or more, particularly one or two, amino acids from the C-terminus of the heavy chain, often a Gly-Lys. This cleavage can occur, for instance, as a result of the process of production of the antibody in a host cell. An antibody produced by expression of a specific nucleic acid molecule encoding a full-length heavy chain can include the full-length heavy chain, or it can include a cleaved variant of the full-length heavy chain, such as a heavy chain lacking a C-terminal Lys or a C-terminal Gly-Lys.

Other types of post-translational modifications can occur during production of antibodies, or otherwise in vivo, such as the modification of an amino acid side chain. For instance, an N-terminal Glu or Gln residue on an antibody chain can be post-translationally modified to an N-terminal pyroglutamate (also known as pyrrolidine carboxylate; abbreviated pE).

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The term “signal sequence” or “leader sequence” refers to a sequence of amino acid residues located at the N terminus of a polypeptide that facilitates secretion of a polypeptide from a mammalian cell. A leader sequence may be cleaved upon export of the polypeptide from the mammalian cell, forming a mature protein. Leader sequences may be natural or synthetic, and they may be heterologous or homologous to the protein to which they are attached. Nonlimiting exemplary leader sequences also include leader sequences from heterologous proteins. In some embodiments, an antibody lacks a leader sequence. In some embodiments, an antibody comprises at least one leader sequence, which may be selected from native antibody leader sequences and heterologous leader sequences.

The term “nucleic acid molecule” or “polynucleotide” includes any compound and/or substance that comprises a polymer of nucleotides. Each nucleotide is composed of a base, specifically a purine- or pyrimidine base (i.e. cytosine I, guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA, circular RNA) vectors, can be unmodified or modified.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-PAD4 antibody” refers to one or more nucleic acid molecules encoding anti-PAD4 antibody heavy and light chains (or fragments thereof), including such nucleic acid molecule(s) in a single vector or separate vectors, and such nucleic acid molecule(s) present at one or more locations in a host cell.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The terms “host cell”, “host cell line”, and “host cell culture” are used interchangeably and refer to cells into which exogenous nucleic acid has been introduced, including the progeny of such cells.

In this disclosure, “binds” or “binding” or “specific binding” and similar terms, when referring to a protein and its ligand or an antibody and its antigen target for example, or some other binding pair, means that the binding affinity between the members of the binding pair is sufficiently strong that the interaction cannot be due to random molecular associations (i.e. “nonspecific binding”). Such binding typically requires a dissociation constant (K_{D}) of 1 μM or less, and may often involve a K_{D} of 100 nM or less.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). Affinity can generally be represented by the dissociation constant (K_{D}). Affinity of an antibody for an antigen can be measured by common methods known in the art, such as surface plasmon resonance (SPR), for instance.

The term “agonist” as used herein refers to a substance, such as an antibody, that causes an increase in at least one activity or function of a molecule to which it binds, or otherwise activates or helps to activate the molecule. The term “antagonist” or “inhibitor” as used herein refers to a substance, such as an antibody, that causes a decrease in at least one activity or function of a molecule to which it binds, or that otherwise blocks or inhibits at least one activity or function of the molecule.

The terms “inhibition” or “inhibit” more generally refer to a decrease or cessation of any event (such as protein ligand binding) or to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. It is not necessary that the inhibition or reduction be complete. For example, in certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater.

“Treatment” as used herein, covers any administration or application of a therapeutic for disease in a human, and includes inhibiting the disease or progression of the disease or one or more disease symptoms, inhibiting or slowing the disease or its progression or one or more of its symptoms, arresting its development, partially or fully relieving the disease or one or more of its symptoms, or preventing a recurrence of one or more symptoms of the disease.

The terms “subject” and “patient” are used interchangeably herein to refer to a human unless expressly indicated otherwise (i.e., a murine subject or the like).

An “autoimmune disease” or “autoimmune disorder,” as used herein, encompasses a disease characterized by the subject's immune system attacking its own normal cells and tissues, and also encompasses immune-mediated diseases which may or may not be characterized by presence of auto-antibodies. The disclosure provides many nonlimiting examples of autoimmune diseases throughout. Some nonlimiting examples of autoimmune diseases include rheumatoid arthritis (RA), lupus (e.g., systemic lupus erythematosus (SLE)), lupus nephritis, vasculitis (e.g., ANCA-associated vasculitis), thrombosis (e.g., venous thrombosis), and inflammatory bowel disease (IBD) (e.g., ulcerative colitis, Crohn's disease). The terms “disease” and “disorder” are used interchangeably herein. In some embodiments, the autoimmune disease is characterized by the presence of auto-antibodies.

The term “effective amount” or “therapeutically effective amount” refers to an amount of a drug effective for treatment of a disease or disorder in a subject, such as to partially or fully relieve one or more symptoms. In some embodiments, an effective amount refers to an amount effective, at dosages and for periods of time necessary, to achieve the desired therapeutic or prophylactic result.

A “biological sample” as used herein refers to a sample taken from a subject or from an animal. Examples of biological samples include tissue samples and liquid biological samples, such as whole blood, serum, plasma, blood supernatant, or synovial fluid. A biological sample may be taken directly from a subject or may be first chemically or physically modified in some fashion prior to use, for example, in order to assist in analysis of the sample.

### II. Exemplary Anti-PAD4 Antibodies

Provided herein are antibodies that bind specifically to protein arginine deiminase 4 (PAD4). In some embodiments, the antibodies inhibit the activity of PAD4, such as the citrullination of arginine.

Clone 13 and Related Antibodies

The disclosure herein, for example, relates to a group of antibodies based on a murine anti-human antibody called “clone 13,” as described further in Example 1 below, for example. For instance, clone 13 was prepared in its original murine anti-human form, and was then humanized to create a series of antibodies called hz13-1 to hz13-12, of which hz13-5 and hz13-12 were further modified at position D31 to D31E (antibodies hz13-5 D31E and hz13-12 D31E), as described in the Examples herein. A cryo EM study of a clone 13 Fab binding to PAD4 and paratope mapping to identify the portions of the clone 13 variable regions and its humanized variants that directly contact PAD4 provided further structural information as to the parts of clone 13 variable regions and those of its related humanized antibodies that dictate PAD4 binding. (See Examples 12-13 below.) Antibody hz13-5 was also further modified to identify antibodies with pH dependent binding to PAD4. (See subsection below and Example 14.)

For example, in some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of SEQ ID NO: 4, an HCDR2 comprising the amino acid sequence of SEQ ID NO: 5, and an HCDR3 comprising the amino acid sequence of SEQ ID NO: 6; and a light chain variable region (VL) comprising a light chain complementarity determining region 1 (LCDR1) comprising the amino acid sequence of SEQ ID NO: 7, an LCDR2 comprising the amino acid sequence of SEQ ID NO: 8, and an LCDR3 comprising the amino acid sequence of SEQ ID NO: 9.

In some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of SEQ ID NO: 62, an HCDR2 comprising the amino acid sequence of SEQ ID NO: 5, and an HCDR3 comprising the amino acid sequence of SEQ ID NO: 6; and a light chain variable region (VL) comprising a light chain complementarity determining region 1 (LCDR1) comprising the amino acid sequence of SEQ ID NO: 7, an LCDR2 comprising the amino acid sequence of SEQ ID NO: 8, and an LCDR3 comprising the amino acid sequence of SEQ ID NO: 9.

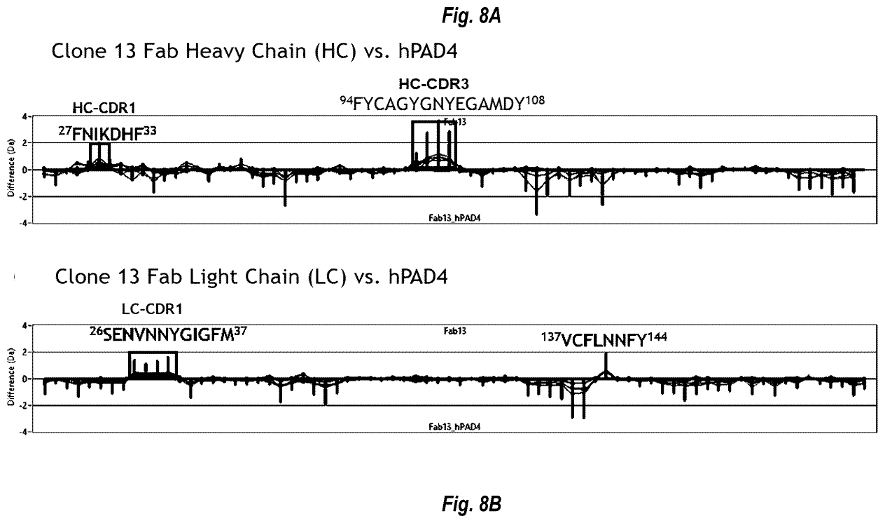

In some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of SEQ ID NO: 4 or 62 and an HCDR3 comprising the amino acid sequence of SEQ ID NO: 6; and a light chain variable region (VL) comprising a light chain complementarity determining region 1 (LCDR1) comprising the amino acid sequence of SEQ ID NO: 7. In particular, paratope mapping and structural analysis of antibodies comprising the above sets of heavy and light chain CDRs revealed that it is the HCDR1, HCDR3, and LCDR1 that make contact with PAD4. (See FIGS. 8A-8B.) Hence, in some embodiments, antibodies herein comprise the above set of these three CDRs. In some embodiments, the antibodies comprise a VH comprising a HCDR1 comprising the amino acid sequence of SEQ ID NO: 4 and an HCDR3 comprising the amino acid sequence of SEQ ID NO: 6; and a VL comprising an LCDR1 comprising the amino acid sequence of SEQ ID NO: 7. In other embodiments, the antibodies comprise a VH comprising a HCDR1 comprising the amino acid sequence of SEQ ID NO: 62 and an HCDR3 comprising the amino acid sequence of SEQ ID NO: 6; and a VL comprising an LCDR1 comprising the amino acid sequence of SEQ ID NO: 7.

In some embodiments, the antibody comprises a heavy chain variable region (VH) comprising the amino acid sequence of SEQ ID NO: 221 or 225 and the amino acid sequence of SEQ ID NO: 222; and a light chain variable region (VL) comprising the amino acid sequence of SEQ ID NO: 223. Each of these paratope regions was identified as contacting PAD4, as described in the Examples and in FIG. 8A-8B. In some embodiments, the antibody further comprises a light chain constant region comprising the amino acid sequence of SEQ ID NO: 224.

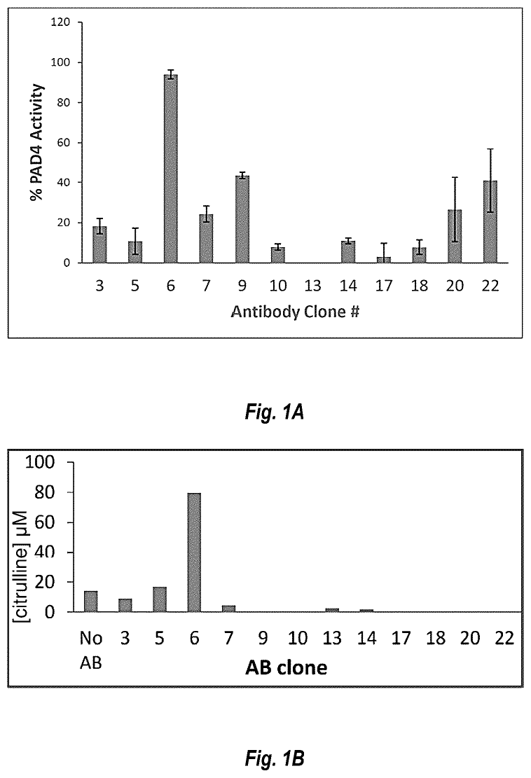

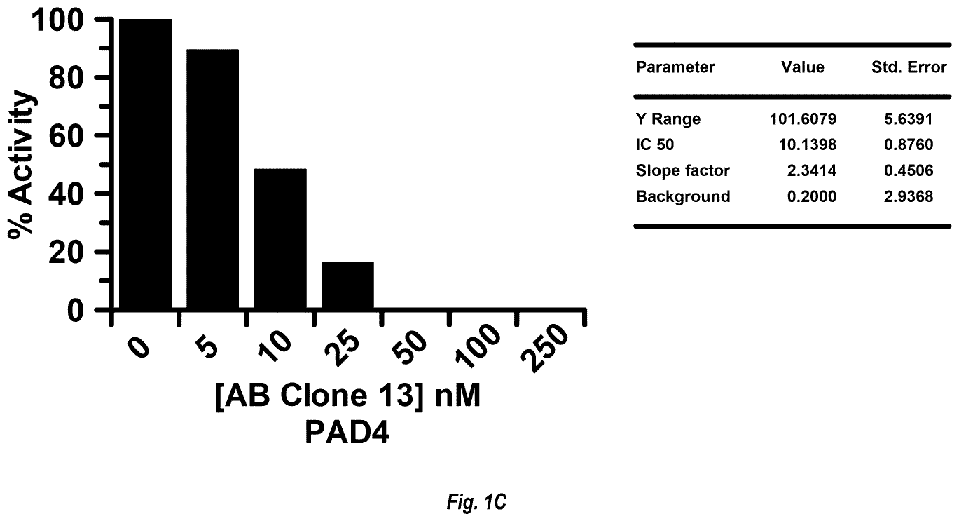

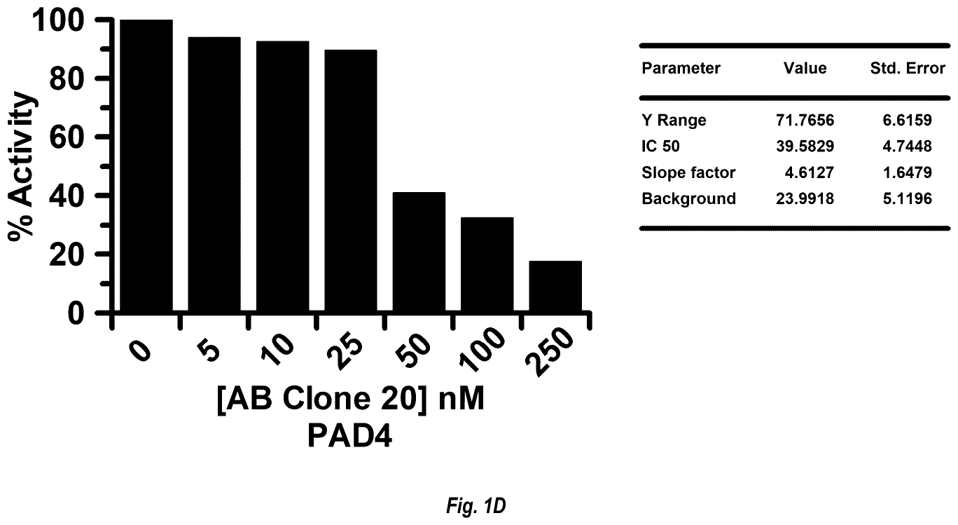

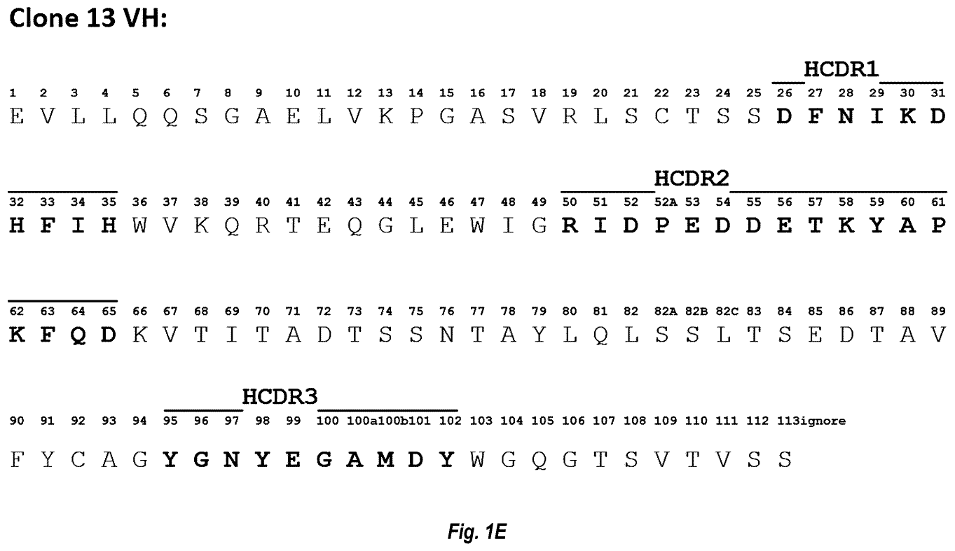

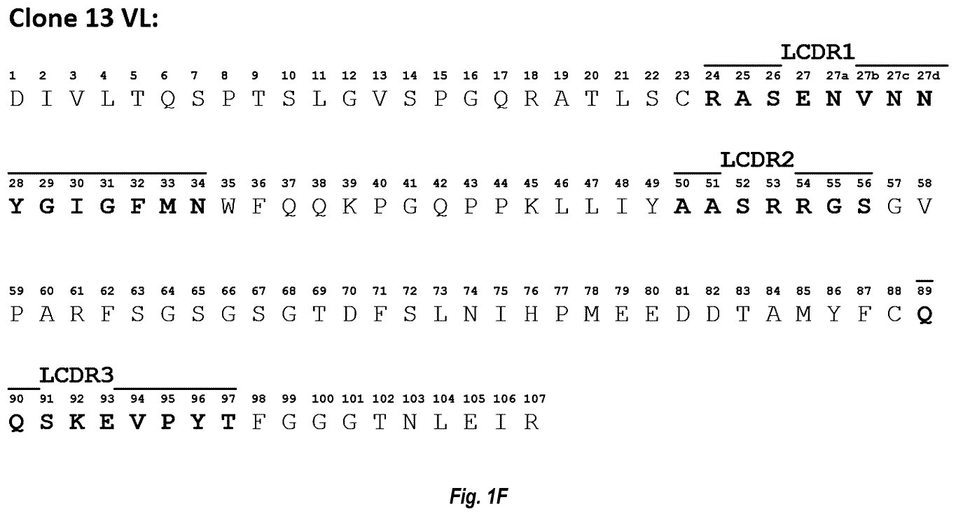

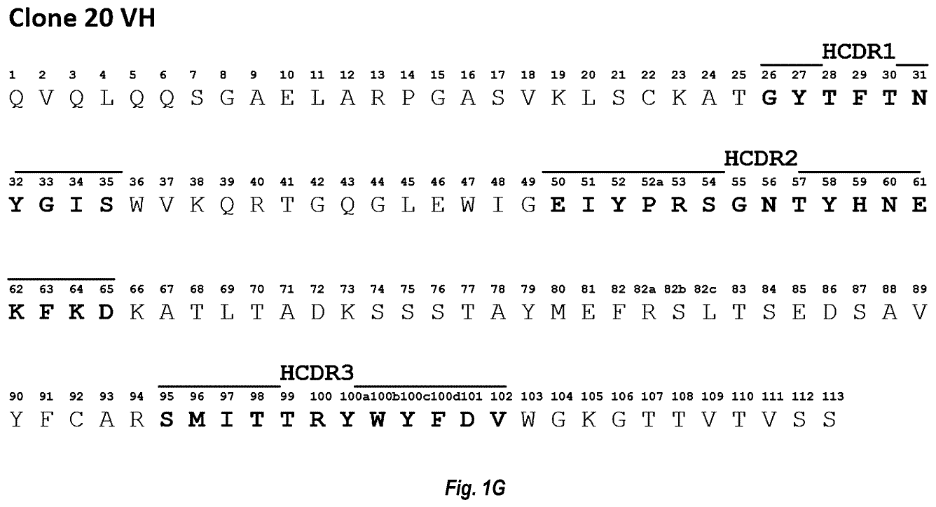

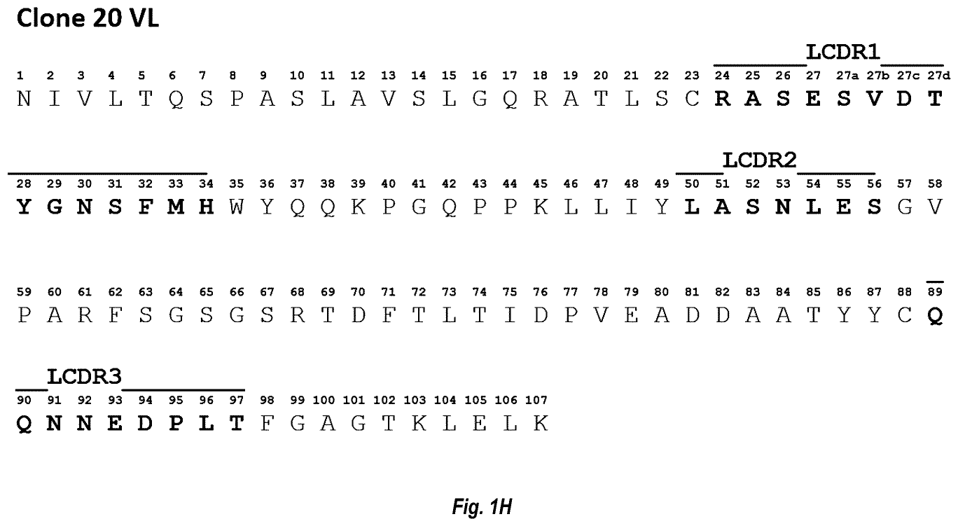

In some embodiments, the antibody comprises a VH comprising a glycine at Kabat position 94 (Gly94). (See FIG. 1E.) Thus, for example, the originally identified mouse anti-human clone 13 antibody comprises a glycine at Kabat position 94, but several human framework regions do not. Thus, in certain humanized antibodies, a back mutation is necessary to obtain a glycine at Kabat position 94. Gly94 is an amino acid residue in the loop adjacent to the VH CDR3 (i.e., the VH CDR3 loop). In some embodiments, Gly94 is implicated in the flexibility and/or the geometry of the VH CDR3 loop. In some embodiments, Gly94 may interact with VH CDR3. In some embodiments, Gly94 is implicated in the activity of the anti-PAD4 antibody. In some embodiments, mutation of Gly94 to a different amino acid, such as a threonine, results in a decrease in flexibility in the VH CDR3 loop, a change in geometry in the VH CDR3 loop, a decrease in interaction between the amino acid at position 94 and the VH CDR3 loop, a decrease in binding of the anti-PAD4 antibody to PAD4, a decrease in activity of the anti-PAD4 antibody, an increase in binding of the anti-PAD4 antibody to extracellular matrix (ECM) proteins, a change in anti-PAD4 antibody secondary structure, a decrease in the stability of the anti-PAD4 antibody, or any combination thereof. Accordingly, in some embodiments, the VH of the antibody comprises a glycine at Kabat position 94 (position 98 of SEQ ID NO: 10). (See FIG. 1E for a depiction of this residue position located just prior to HCDR3.)

In some embodiments, the antibody comprises a VH that is at least 90%, at least 95%, at least 97%, or at least 99% identical to the amino acid sequence of any one of SEQ ID NOs: 10, 14, 18, 22, 26, 30, 34, 38, 42, 46, 50, 54, 58, or 68. In some embodiments, the antibody comprises a VL that is at least 90%, at least 95%, at least 97%, or at least 99% identical to the amino acid sequence of any one of SEQ ID NOs: 12, 16, 20, 24, 28, 32, 36, 40, 44, 48, 52, 56, 60, or 70. In some embodiments, the antibody comprises both a VH that is at least 90%, at least 95%, at least 97%, or at least 99% identical to the amino acid sequence of any one of SEQ ID NOs: 10, 14, 18, 22, 26, 30, 34, 38, 42, 46, 50, 54, 58, or 68; and a VL that is at least 90%, at least 95%, at least 97%, or at least 99% identical to the amino acid sequence of any one of SEQ ID NOs: 12, 16, 20, 24, 28, 32, 36, 40, 44, 48, 52, 56, 60, or 70. In some embodiments, the antibody comprises a VH comprising the amino acid sequence of any one of SEQ ID Nos: 10, 14, 18, 22, 26, 30, 34, 38, 42, 46, 50, 54, 58, or 68 modified by 1-10 amino acid substitutions, 1-5 amino acid substitutions, or 1-3 amino acid substitutions. In some embodiments, the antibody comprises a VL comprising the amino acid sequence of any one of SEQ ID Nos: 12, 16, 20, 24, 28, 32, 36, 40, 44, 48, 52, 56, 60, or 70 modified by 1-10 amino acid substitutions, 1-5 amino acid substitutions, or 1-3 amino acid substitutions. In some embodiments, the antibody comprises a both a VH comprising the amino acid sequence of any one of SEQ ID Nos: 10, 14, 18, 22, 26, 30, 34, 38, 42, 46, 50, 54, 58, or 68 modified by 1-10 amino acid substitutions, 1-5 amino acid substitutions, or 1-3 amino acid substitutions and a VL comprising the amino acid sequence of any one of SEQ ID Nos: 12, 16, 20, 24, 28, 32, 36, 40, 44, 48, 52, 56, 60, or 70 modified by 1-10 amino acid substitutions, 1-5 amino acid substitutions, or 1-3 amino acid substitutions. In some such cases, the antibody VH and VL further comprise (a) an HCDR1 comprising the amino acid sequence of SEQ ID NO: 4 or 62 and an HCDR3 comprising the amino acid sequence of SEQ ID NO: 6 and an LCDR1 comprising the amino acid sequence of SEQ ID NO: 7, (b) an HCDR1 comprising the amino acid sequence of SEQ ID NO: 4 or 62, an HCDR2 comprising the amino acid sequence of SEQ ID NO: 5, an HCDR3 comprising the amino acid sequence of SEQ ID NO: 6, an LCDR1 comprising the amino acid sequence of SEQ ID NO: 7, an LCDR2 comprising the amino acid sequence of SEQ ID NO: 8 an LCDR3 comprising the amino acid sequence of SEQ ID NO: 9, or (c) the amino acid sequence of SEQ ID NO: 221 or 225 in the VH and the amino acid sequence of SEQ ID NO: 222 in the VL, further optionally with a light chain constant region comprising the amino acid sequence of SEQ ID NO: 224. Thus, in such cases, the variation in the VH and VL compared to the above listed sequence identification numbers is located in regions outside of these specific CDR or paratope sequences. In some embodiments, the antibody comprises a VH comprising the amino acid sequence of any one of SEQ ID Nos: 10, 14, 18, 22, 26, 30, 34, 38, 42, 46, 50, 54, 58, or 68. In some embodiments, the antibody comprises a VL comprising the amino acid sequence of any one of SEQ ID Nos: 12, 16, 20, 24, 28, 32, 36, 40, 44, 48, 52, 56, 60, or 70.

In yet further embodiments, the antibody comprises both a VH comprising the amino acid sequence of any one of SEQ ID Nos: 10, 14, 18, 22, 26, 30, 34, 38, 42, 46, 50, 54, 58, or 68; and a VL comprising the amino acid sequence of any one of SEQ ID Nos: 12, 16, 20, 24, 28, 32, 36, 40, 44, 48, 52, 56, 60, or 70. Thus, for example, the following exemplary antibodies are within the scope of this disclosure:
An isolated antibody that specifically binds to protein arginine deiminase 4 (PAD4), wherein the antibody comprises a VH comprising the amino acid sequence of SEQ ID NO: 10 and a VL comprising the amino acid sequence of SEQ ID NO: 12;
An isolated antibody that specifically binds to protein arginine deiminase 4 (PAD4), wherein the antibody comprises a VH comprising the amino acid sequence of SEQ ID NO: 14 and a VL comprising the amino acid sequence of SEQ ID NO: 16;
An isolated antibody that specifically binds to protein arginine deiminase 4 (PAD4), wherein the antibody comprises a VH comprising the amino acid sequence of SEQ ID NO: 18 and a VL comprising the amino acid sequence of SEQ ID NO: 20;
An isolated antibody that specifically binds to protein arginine deiminase 4 (PAD4), wherein the antibody comprises a VH comprising the amino acid sequence of SEQ ID NO: 22 and a VL comprising the amino acid sequence of SEQ ID NO: 24;
An isolated antibody that specifically binds to protein arginine deiminase 4 (PAD4), wherein the antibody comprises a VH comprising the amino acid sequence of SEQ ID NO: 26 and a VL comprising the amino acid sequence of SEQ ID NO: 28;
An isolated antibody that specifically binds to protein arginine deiminase 4 (PAD4), wherein the antibody comprises a VH comprising the amino acid sequence of SEQ ID NO: 30 and a VL comprising the amino acid sequence of SEQ ID NO: 32;
An isolated antibody that specifically binds to protein arginine deiminase 4 (PAD4), wherein the antibody comprises a VH comprising the amino acid sequence of SEQ ID NO: 34 and a VL comprising the amino acid sequence of SEQ ID NO: 36;
An isolated antibody that specifically binds to protein arginine deiminase 4 (PAD4), wherein the antibody comprises a VH comprising the amino acid sequence of SEQ ID NO: 38 and a VL comprising the amino acid sequence of SEQ ID NO: 40;
An isolated antibody that specifically binds to protein arginine deiminase 4 (PAD4), wherein the antibody comprises a VH comprising the amino acid sequence of SEQ ID NO: 42 and a VL comprising the amino acid sequence of SEQ ID NO: 44;
An isolated antibody that specifically binds to protein arginine deiminase 4 (PAD4), wherein the antibody comprises a VH comprising the amino acid sequence of SEQ ID NO: 46 and a VL comprising the amino acid sequence of SEQ ID NO: 48;
An isolated antibody that specifically binds to protein arginine deiminase 4 (PAD4), wherein the antibody comprises a VH comprising the amino acid sequence of SEQ ID NO: 50 and a VL comprising the amino acid sequence of SEQ ID NO: 52;
An isolated antibody that specifically binds to protein arginine deiminase 4 (PAD4), wherein the antibody comprises a VH comprising the amino acid sequence of SEQ ID NO: 54 and a VL comprising the amino acid sequence of SEQ ID NO: 56;
An isolated antibody that specifically binds to protein arginine deiminase 4 (PAD4), wherein the antibody comprises a VH comprising the amino acid sequence of SEQ ID NO: 58 and a VL comprising the amino acid sequence of SEQ ID NO: 60;
An isolated antibody that specifically binds to protein arginine deiminase 4 (PAD4), wherein the antibody comprises a VH comprising the amino acid sequence of SEQ ID NO: 68 and a VL comprising the amino acid sequence of SEQ ID NO: 70.

In any of the above antibodies, in some cases the antibody binds to an epitope on PAD4 comprising SEQ ID NO: 217 and SEQ ID NO: 218.

In some embodiments herein, the antibody is an IgA, IgG, or IgM antibody. In some cases, the antibody is an IgG antibody, such as a human IgG1, IgG2, IgG3, or IgG4 antibody or a murine IgG1 or IgG2 antibody. In some cases, the antibody comprises a wild-type, human IgG1, IgG2, or IgG4 heavy chain constant region. In some embodiments, the antibody comprises a full length heavy chain and/or a full length light chain. In other cases, the antibody lacks a C-terminal lysine at the end of the heavy chain constant region. In yet other cases, the antibody lacks a C-terminal glycine-lysine at the end of the heavy chain constant region. In some cases, the antibody is an antibody fragment, such as an Fv, single-chain Fv (scFv), Fab, Fab′, or (Fab′)₂.

In some embodiments, the antibody is a bispecific or multispecific antibody, or which is conjugated covalently or noncovalently to at least one other molecule. In some embodiments, the antibody is conjugated covalently or noncovalently to at least one other molecule, wherein the at least one other molecule comprises a detection label and/or a drug.

In some embodiments, the antibody comprises a human IgG1 heavy chain constant region comprising an amino acid sequence at least 90%, at least 95%, at least 97%, or at least 99% identical to the amino acid sequence of any one of SEQ ID Nos: 174, 176, 178, 180, 182, 184, 186, 188, 190, or 192. In some embodiments, the antibody comprises a light chain constant region comprising an amino acid sequence at least 90%, at least 95%, at least 97%, or at least 99% identical to the amino acid sequence of SEQ ID NO: 194. In some embodiments, the antibody comprises both a human IgG1 heavy chain constant region comprising an amino acid sequence at least 90%, at least 95%, at least 97%, or at least 99% identical to the amino acid sequence of any one of SEQ ID Nos: 174, 176, 178, 180, 182, 184, 186, 188, 190, or 192; and a light chain constant region comprising an amino acid sequence at least 90%, at least 95%, at least 97%, or at least 99% identical to the amino acid sequence of SEQ ID NO: 194. In some cases, the antibody comprises a heavy chain constant region comprising an amino acid sequence modified by 1-10 amino acid substitutions, 1-5 amino acid substitutions, or 1-3 amino acid substitutions compared to the amino acid sequence of any one of SEQ ID Nos: 174, 176, 178, 180, 182, 184, 186, 188, 190, or 192. In some cases, the antibody comprises a light chain constant region comprising an amino acid sequence modified by 1-10 amino acid substitutions, 1-5 amino acid substitutions, or 1-3 amino acid substitutions compared to the amino acid sequence of SEQ ID NO: 194. In some cases, the antibody comprises both a heavy chain constant region comprising an amino acid sequence modified by 1-10 amino acid substitutions, 1-5 amino acid substitutions, or 1-3 amino acid substitutions compared to the amino acid sequence of any one of SEQ ID Nos: 174, 176, 178, 180, 182, 184, 186, 188, 190, or 192; and a light chain constant region comprising an amino acid sequence modified by 1-10 amino acid substitutions, 1-5 amino acid substitutions, or 1-3 amino acid substitutions compared to the amino acid sequence of SEQ ID NO: 194. In some embodiments, the antibody comprises a human IgG1 heavy chain constant region comprising amino acid sequence of any one of SEQ ID Nos: 174, 176, 178, 180, 182, 184, 186, 188, 190, or 192. In some embodiments, the antibody comprises a light chain constant region comprising the amino acid sequence of SEQ ID NO: 194. In some embodiments, the antibody comprises both a human IgG1 heavy chain constant region comprising the amino acid sequence of any one of SEQ ID Nos: 174, 176, 178, 180, 182, 184, 186, 188, 190, or 192; and a light chain constant region comprising the amino acid sequence of SEQ ID NO: 194.

Clone 20 and Related Antibodies

The disclosure further relates to a second mouse anti-human antibody, clone 20, and its humanized variants hz20-1 to hz20-14, as described in the Examples below. Accordingly, in some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of SEQ ID NO: 72, an HCDR2 comprising the amino acid sequence of SEQ ID NO: 73, and an HCDR3 comprising the amino acid sequence of SEQ ID NO: 74; and a light chain variable region (VL) comprising a light chain complementarity determining region 1 (LCDR1) comprising the amino acid sequence of SEQ ID NO: 75, an LCDR2 comprising the amino acid sequence of SEQ ID NO: 76, and an LCDR3 comprising the amino acid sequence of SEQ ID NO: 77.

In some embodiments, the antibody comprises a VH that is at least 90%, at least 95%, at least 97%, or at least 99% identical to the amino acid sequence of any one of SEQ ID NOs: 78, 82, 86, 90, 94, 98, 102, 106, 110, 114, 118, 122, 126, 130, or 134. In some embodiments, the antibody comprises a VL that is at least 90%, at least 95%, at least 97%, or at least 99% identical to the amino acid sequence of any one of SEQ ID NOs: 80, 84, 88, 92, 96, 100, 104, 108, 112, 116, 120, 124, 128, 132, or 136. In some embodiments, the antibody comprises a VH comprising the amino acid sequence of any one of SEQ ID Nos: 78, 82, 86, 90, 94, 98, 102, 106, 110, 114, 118, 122, 126, 130, or 134 modified by 1-10 amino acid substitutions, 1-5 amino acid substitutions, or 1-3 amino acid substitutions; and wherein the antibody comprises a VL comprising the amino acid sequence of any one of SEQ ID Nos: 80, 84, 88, 92, 96, 100, 104, 108, 112, 116, 120, 124, 128, 132, or 136 modified by 1-10 amino acid substitutions, 1-5 amino acid substitutions, or 1-3 amino acid substitutions. In some such cases, the antibody VH further comprises an HCDR1 comprising the amino acid sequence of SEQ ID NO: 72, an HCDR2 comprising the amino acid sequence of SEQ ID NO: 73, or an HCDR3 comprising the amino acid sequence of SEQ ID NO: 74. In some cases, the antibody VL further comprises an LCDR1 comprising the amino acid sequence of SEQ ID NO: 75, an LCDR2 comprising the amino acid sequence of SEQ ID NO: 76, or an LCDR3 comprising the amino acid sequence of SEQ ID NO: 77. In some embodiments, the antibody comprises a VH comprising the amino acid sequence of any one of SEQ ID Nos: 78, 82, 86, 90, 94, 98, 102, 106, 110, 114, 118, 122, 126, 130, or 134. In some embodiments, the antibody comprises a VL comprising the amino acid sequence of any one of SEQ ID Nos: 80, 84, 88, 92, 96, 100, 104, 108, 112, 116, 120, 124, 128, 132, or 136.

In yet further embodiments, the antibody comprises a VH comprising the amino acid sequence of any one of SEQ ID Nos: 78, 82, 86, 90, 94, 98, 102, 106, 110, 114, 118, 122, 126, 130, or 134; and comprises a VL comprising the amino acid sequence of any one of SEQ ID Nos: 80, 84, 88, 92, 96, 100, 104, 108, 112, 116, 120, 124, 128, 132, or 136. Thus, for example, the following exemplary antibodies are within the scope of this disclosure:
An isolated antibody that specifically binds to protein arginine deiminase 4 (PAD4), wherein the antibody comprises a VH comprising the amino acid sequence of SEQ ID NO: 78 and a VL comprising the amino acid sequence of SEQ ID NO: 80;
An isolated antibody that specifically binds to protein arginine deiminase 4 (PAD4), wherein the antibody comprises a VH comprising the amino acid sequence of SEQ ID NO: 82 and a VL comprising the amino acid sequence of SEQ ID NO: 84;
An isolated antibody that specifically binds to protein arginine deiminase 4 (PAD4), wherein the antibody comprises a VH comprising the amino acid sequence of SEQ ID NO: 86 and a VL comprising the amino acid sequence of SEQ ID NO: 88;
An isolated antibody that specifically binds to protein arginine deiminase 4 (PAD4), wherein the antibody comprises a VH comprising the amino acid sequence of SEQ ID NO: 90 and a VL comprising the amino acid sequence of SEQ ID NO: 92;
An isolated antibody that specifically binds to protein arginine deiminase 4 (PAD4), wherein the antibody comprises a VH comprising the amino acid sequence of SEQ ID NO: 94 and a VL comprising the amino acid sequence of SEQ ID NO: 96;
An isolated antibody that specifically binds to protein arginine deiminase 4 (PAD4), wherein the antibody comprises a VH comprising the amino acid sequence of SEQ ID NO: 98 and a VL comprising the amino acid sequence of SEQ ID NO: 100;
An isolated antibody that specifically binds to protein arginine deiminase 4 (PAD4), wherein the antibody comprises a VH comprising the amino acid sequence of SEQ ID NO: 102 and a VL comprising the amino acid sequence of SEQ ID NO: 104;
An isolated antibody that specifically binds to protein arginine deiminase 4 (PAD4), wherein the antibody comprises a VH comprising the amino acid sequence of SEQ ID NO: 106 and a VL comprising the amino acid sequence of SEQ ID NO: 108;
An isolated antibody that specifically binds to protein arginine deiminase 4 (PAD4), wherein the antibody comprises a VH comprising the amino acid sequence of SEQ ID NO: 110 and a VL comprising the amino acid sequence of SEQ ID NO: 112;
An isolated antibody that specifically binds to protein arginine deiminase 4 (PAD4), wherein the antibody comprises a VH comprising the amino acid sequence of SEQ ID NO: 114 and a VL comprising the amino acid sequence of SEQ ID NO: 116;
An isolated antibody that specifically binds to protein arginine deiminase 4 (PAD4), wherein the antibody comprises a VH comprising the amino acid sequence of SEQ ID NO: 118 and a VL comprising the amino acid sequence of SEQ ID NO: 120;
An isolated antibody that specifically binds to protein arginine deiminase 4 (PAD4), wherein the antibody comprises a VH comprising the amino acid sequence of SEQ ID NO: 122 and a VL comprising the amino acid sequence of SEQ ID NO: 124;
An isolated antibody that specifically binds to protein arginine deiminase 4 (PAD4), wherein the antibody comprises a VH comprising the amino acid sequence of SEQ ID NO: 126 and a VL comprising the amino acid sequence of SEQ ID NO: 128;
An isolated antibody that specifically binds to protein arginine deiminase 4 (PAD4), wherein the antibody comprises a VH comprising the amino acid sequence of SEQ ID NO: 130 and a VL comprising the amino acid sequence of SEQ ID NO: 132;
An isolated antibody that specifically binds to protein arginine deiminase 4 (PAD4), wherein the antibody comprises a VH comprising the amino acid sequence of SEQ ID NO: 134 and a VL comprising the amino acid sequence of SEQ ID NO: 136.

In any of the above antibodies, in some cases, the antibody binds to an epitope on PAD4 comprising SEQ ID NO: 219 and SEQ ID NO: 220.

In some embodiments herein, the antibody is an IgA, IgG, or IgM antibody. In some cases, the antibody is an IgG antibody, such as a human IgG1, IgG2, IgG3, or IgG4 antibody or a murine IgG1 or IgG2 antibody. In some cases, the antibody comprises a wild-type, human IgG1, IgG2, or IgG4 heavy chain constant region. In certain aspects, the antibody is of the human IgG1 isotype. In certain aspects, the antibody is of the human IgG1 isotype with a P329G, L234A and L235A (LALAPG; EU numbering) mutation to reduce Fc-region effector function. In other aspects, the antibody is of the human IgG2 isotype. In certain aspects, the antibody is of the IgG4 isotype with an S228P mutation (EU numbering) in the hinge region to improve stability of IgG4 antibody. In some aspects, the antibody (e.g., a non-humanized antibody) may have a non-human IgG constant region, and may be, for example, a murine IgG2a antibody such as a murine IgG2a LALAPG antibody. In some embodiments, the antibody comprises a full length heavy chain and/or a full length light chain. In other cases, the antibody lacks a C-terminal lysine at the end of the heavy chain constant region. In yet other cases, the antibody lacks a C-terminal glycine-lysine at the end of the heavy chain constant region. In some cases, the antibody is an antibody fragment, such as an Fv, single-chain Fv (scFv), Fab, Fab′, or (Fab′)₂.

In some embodiments, the antibody is a bispecific or multispecific antibody, or which is conjugated covalently or noncovalently to at least one other molecule. In some embodiments, the antibody is conjugated covalently or noncovalently to at least one other molecule, wherein the at least one other molecule comprises a detection label and/or a drug.

In some embodiments, the antibody comprises a human IgG1 heavy chain constant region comprising an amino acid sequence at least 90%, at least 95%, at least 97%, or at least 99% identical to the amino acid sequence of any one of SEQ ID Nos: 174, 176, 178, 180, 182, 184, 186, 188, 190, or 192. In some embodiments, the antibody comprises a light chain constant region comprising an amino acid sequence at least 90%, at least 95%, at least 97%, or at least 99% identical to the amino acid sequence of SEQ ID NO: 194. In some embodiments, the antibody comprises both a human IgG1 heavy chain constant region comprising an amino acid sequence at least 90%, at least 95%, at least 97%, or at least 99% identical to the amino acid sequence of any one of SEQ ID Nos: 174, 176, 178, 180, 182, 184, 186, 188, 190, or 192; and a light chain constant region comprising an amino acid sequence at least 90%, at least 95%, at least 97%, or at least 99% identical to the amino acid sequence of SEQ ID NO: 194. In some cases, the antibody comprises a heavy chain constant region comprising an amino acid sequence modified by 1-10 amino acid substitutions, 1-5 amino acid substitutions, or 1-3 amino acid substitutions compared to the amino acid sequence of any one of SEQ ID Nos: 174, 176, 178, 180, 182, 184, 186, 188, 190, or 192. In some cases, the antibody comprises a light chain constant region comprising an amino acid sequence modified by 1-10 amino acid substitutions, 1-5 amino acid substitutions, or 1-3 amino acid substitutions compared to the amino acid sequence of SEQ ID NO: 194. In some cases, the antibody comprises both a heavy chain constant region comprising an amino acid sequence modified by 1-10 amino acid substitutions, 1-5 amino acid substitutions, or 1-3 amino acid substitutions compared to the amino acid sequence of any one of SEQ ID Nos: 174, 176, 178, 180, 182, 184, 186, 188, 190, or 192; and a light chain constant region comprising an amino acid sequence modified by 1-10 amino acid substitutions, 1-5 amino acid substitutions, or 1-3 amino acid substitutions compared to the amino acid sequence of SEQ ID NO: 194. In some embodiments, the antibody comprises a human IgG1 heavy chain constant region comprising amino acid sequence of any one of SEQ ID Nos: 174, 176, 178, 180, 182, 184, 186, 188, 190, or 192. In some embodiments, the antibody comprises a light chain constant region comprising the amino acid sequence of SEQ ID NO: 194. In some embodiments, the antibody comprises both a human IgG1 heavy chain constant region comprising the amino acid sequence of any one of SEQ ID Nos: 174, 176, 178, 180, 182, 184, 186, 188, 190, or 192; and a light chain constant region comprising the amino acid sequence of SEQ ID NO: 194.

pH Dependent Clone 13-Related Antibodies

In some embodiments, the disclosure relates to one or more antibody variants of parental antibody clone 13 or hz13-5, in which particular residues are modified as described below in Example 14 below. In certain cases, though not in all cases, these modifications were shown to impact the pH dependence of PAD4 binding, as described in Example 14.

In some embodiments, the disclosure relates to an isolated antibody that specifically binds to protein arginine deiminase 4 (PAD4), wherein the antibody comprises a VH comprising an HCDR1 comprising the amino acid sequence of positions 26-35, an HCDR2 comprising the amino acid sequence of positions 50-66, and an HCDR3 comprising the amino acid sequence of positions 99-108 of SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, or SEQ ID NO: 168. In some embodiments above, the antibody further comprises a VL comprising an LCDR1 comprising the amino acid sequence of residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170. In other embodiments above, the antibody further comprises a VL comprising an LCDR1 comprising the amino acid sequence of residues 24-38 of SEQ ID NO: 172, an LCDR2 comprising the amino acid sequence of residues 54-60 of SEQ ID NO: 172, and an LCDR3 comprising the amino acid sequence of residues 93-101 of SEQ ID NO: 172.

In some embodiments, the antibody comprises:
a VH comprising an HCDR1 comprising the amino acid sequence of positions 26-35 of SEQ ID NO: 138, an HCDR2 comprising the amino acid sequence of positions 50-66 of SEQ ID NO: 138, and an HCDR3 comprising the amino acid sequence of positions 99-108 of SEQ ID NO: 138, and a VL comprising an LCDR1 comprising the amino acid sequence of residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170;
a VH comprising an HCDR1 comprising the amino acid sequence of positions 26-35 of SEQ ID NO: 140, an HCDR2 comprising the amino acid sequence of positions 50-66 of SEQ ID NO: 140, and an HCDR3 comprising the amino acid sequence of positions 99-108 of SEQ ID NO: 140, and a VL comprising an LCDR1 comprising the amino acid sequence of residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170;
a VH comprising an HCDR1 comprising the amino acid sequence of positions 26-35 of SEQ ID NO: 142, an HCDR2 comprising the amino acid sequence of positions 50-66 of SEQ ID NO: 142, and an HCDR3 comprising the amino acid sequence of positions 99-108 of SEQ ID NO: 142, and a VL comprising an LCDR1 comprising the amino acid sequence of residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170;
a VH comprising an HCDR1 comprising the amino acid sequence of positions 26-35 of SEQ ID NO: 144, an HCDR2 comprising the amino acid sequence of positions 50-66 of SEQ ID NO: 144, and an HCDR3 comprising the amino acid sequence of positions 99-108 of SEQ ID NO: 144, and a VL comprising an LCDR1 comprising the amino acid sequence of residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170;
a VH comprising an HCDR1 comprising the amino acid sequence of positions 26-35 of SEQ ID NO: 146, an HCDR2 comprising the amino acid sequence of positions 50-66 of SEQ ID NO: 146, and an HCDR3 comprising the amino acid sequence of positions 99-108 of SEQ ID NO: 146, and a VL comprising an LCDR1 comprising the amino acid sequence of residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170;
a VH comprising an HCDR1 comprising the amino acid sequence of positions 26-35 of SEQ ID NO: 148, an HCDR2 comprising the amino acid sequence of positions 50-66 of SEQ ID NO: 148, and an HCDR3 comprising the amino acid sequence of positions 99-108 of SEQ ID NO: 148, and a VL comprising an LCDR1 comprising the amino acid sequence of residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170;
a VH comprising an HCDR1 comprising the amino acid sequence of positions 26-35 of SEQ ID NO: 150, an HCDR2 comprising the amino acid sequence of positions 50-66 of SEQ ID NO: 150, and an HCDR3 comprising the amino acid sequence of positions 99-108 of SEQ ID NO: 150, and a VL comprising an LCDR1 comprising the amino acid sequence of residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170;
a VH comprising an HCDR1 comprising the amino acid sequence of positions 26-35 of SEQ ID NO: 152, an HCDR2 comprising the amino acid sequence of positions 50-66 of SEQ ID NO: 152, and an HCDR3 comprising the amino acid sequence of positions 99-108 of SEQ ID NO: 152, and a VL comprising an LCDR1 comprising the amino acid sequence of residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170;
a VH comprising an HCDR1 comprising the amino acid sequence of positions 26-35 of SEQ ID NO: 154, an HCDR2 comprising the amino acid sequence of positions 50-66 of SEQ ID NO: 154, and an HCDR3 comprising the amino acid sequence of positions 99-108 of SEQ ID NO: 154, and a VL comprising an LCDR1 comprising the amino acid sequence of residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170;
a VH comprising an HCDR1 comprising the amino acid sequence of positions 26-35 of SEQ ID NO: 156, an HCDR2 comprising the amino acid sequence of positions 50-66 of SEQ ID NO: 156, and an HCDR3 comprising the amino acid sequence of positions 99-108 of SEQ ID NO: 156, and a VL comprising an LCDR1 comprising the amino acid sequence of residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170;
a VH comprising an HCDR1 comprising the amino acid sequence of positions 26-35 of SEQ ID NO: 158, an HCDR2 comprising the amino acid sequence of positions 50-66 of SEQ ID NO: 158, and an HCDR3 comprising the amino acid sequence of positions 99-108 of SEQ ID NO: 158, and a VL comprising an LCDR1 comprising the amino acid sequence of residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170;
a VH comprising an HCDR1 comprising the amino acid sequence of positions 26-35 of SEQ ID NO: 160, an HCDR2 comprising the amino acid sequence of positions 50-66 of SEQ ID NO: 160, and an HCDR3 comprising the amino acid sequence of positions 99-108 of SEQ ID NO: 160, and a VL comprising an LCDR1 comprising the amino acid sequence of residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170;
a VH comprising an HCDR1 comprising the amino acid sequence of positions 26-35 of SEQ ID NO: 162, an HCDR2 comprising the amino acid sequence of positions 50-66 of SEQ ID NO: 162, and an HCDR3 comprising the amino acid sequence of positions 99-108 of SEQ ID NO: 162, and a VL comprising an LCDR1 comprising the amino acid sequence of residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170;
a VH comprising an HCDR1 comprising the amino acid sequence of positions 26-35 of SEQ ID NO: 164, an HCDR2 comprising the amino acid sequence of positions 50-66 of SEQ ID NO: 164, and an HCDR3 comprising the amino acid sequence of positions 99-108 of SEQ ID NO: 164, and a VL comprising an LCDR1 comprising the amino acid sequence of residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170;
a VH comprising an HCDR1 comprising the amino acid sequence of positions 26-35 of SEQ ID NO: 166, an HCDR2 comprising the amino acid sequence of positions 50-66 of SEQ ID NO: 166, and an HCDR3 comprising the amino acid sequence of positions 99-108 of SEQ ID NO: 166, and a VL comprising an LCDR1 comprising the amino acid sequence of residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170; or
a VH comprising an HCDR1 comprising the amino acid sequence of positions 26-35 of SEQ ID NO: 168, an HCDR2 comprising the amino acid sequence of positions 50-66 of SEQ ID NO: 168, and an HCDR3 comprising the amino acid sequence of positions 99-108 of SEQ ID NO: 168, and a VL comprising an LCDR1 comprising the amino acid sequence of residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170.

In some embodiments, the antibody comprises:
a VH comprising an HCDR1 comprising the amino acid sequence of positions 26-35 of SEQ ID NO: 138, an HCDR2 comprising the amino acid sequence of positions 50-66 of SEQ ID NO: 138, and an HCDR3 comprising the amino acid sequence of positions 99-108 of SEQ ID NO: 138, and a VL comprising an LCDR1 comprising the amino acid sequence of residues 24-38 of SEQ ID NO: 172, an LCDR2 comprising the amino acid sequence of residues 54-60 of SEQ ID NO: 172, and an LCDR3 comprising the amino acid sequence of residues 93-101 of SEQ ID NO: 172;
a VH comprising an HCDR1 comprising the amino acid sequence of positions 26-35 of SEQ ID NO: 140, an HCDR2 comprising the amino acid sequence of positions 50-66 of SEQ ID NO: 140, and an HCDR3 comprising the amino acid sequence of positions 99-108 of SEQ ID NO: 140, and a VL comprising an LCDR1 comprising the amino acid sequence of residues 24-38 of SEQ ID NO: 172, an LCDR2 comprising the amino acid sequence of residues 54-60 of SEQ ID NO: 172, and an LCDR3 comprising the amino acid sequence of residues 93-101 of SEQ ID NO: 172;
a VH comprising an HCDR1 comprising the amino acid sequence of positions 26-35 of SEQ ID NO: 142, an HCDR2 comprising the amino acid sequence of positions 50-66 of SEQ ID NO: 142, and an HCDR3 comprising the amino acid sequence of positions 99-108 of SEQ ID NO: 142, and a VL comprising an LCDR1 comprising the amino acid sequence of residues 24-38 of SEQ ID NO: 172, an LCDR2 comprising the amino acid sequence of residues 54-60 of SEQ ID NO: 172, and an LCDR3 comprising the amino acid sequence of residues 93-101 of SEQ ID NO: 172;
a VH comprising an HCDR1 comprising the amino acid sequence of positions 26-35 of SEQ ID NO: 144, an HCDR2 comprising the amino acid sequence of positions 50-66 of SEQ ID NO: 144, and an HCDR3 comprising the amino acid sequence of positions 99-108 of SEQ ID NO: 144, and a VL comprising an LCDR1 comprising the amino acid sequence of residues 24-38 of SEQ ID NO: 172, an LCDR2 comprising the amino acid sequence of residues 54-60 of SEQ ID NO: 172, and an LCDR3 comprising the amino acid sequence of residues 93-101 of SEQ ID NO: 172;
a VH comprising an HCDR1 comprising the amino acid sequence of positions 26-35 of SEQ ID NO: 146, an HCDR2 comprising the amino acid sequence of positions 50-66 of SEQ ID NO: 146, and an HCDR3 comprising the amino acid sequence of positions 99-108 of SEQ ID NO: 146, and a VL comprising an LCDR1 comprising the amino acid sequence of residues 24-38 of SEQ ID NO: 172, an LCDR2 comprising the amino acid sequence of residues 54-60 of SEQ ID NO: 172, and an LCDR3 comprising the amino acid sequence of residues 93-101 of SEQ ID NO: 172;
a VH comprising an HCDR1 comprising the amino acid sequence of positions 26-35 of SEQ ID NO: 148, an HCDR2 comprising the amino acid sequence of positions 50-66 of SEQ ID NO: 148, and an HCDR3 comprising the amino acid sequence of positions 99-108 of SEQ ID NO: 148, and a VL comprising an LCDR1 comprising the amino acid sequence of residues 24-38 of SEQ ID NO: 172, an LCDR2 comprising the amino acid sequence of residues 54-60 of SEQ ID NO: 172, and an LCDR3 comprising the amino acid sequence of residues 93-101 of SEQ ID NO: 172;
a VH comprising an HCDR1 comprising the amino acid sequence of positions 26-35 of SEQ ID NO: 150, an HCDR2 comprising the amino acid sequence of positions 50-66 of SEQ ID NO: 150, and an HCDR3 comprising the amino acid sequence of positions 99-108 of SEQ ID NO: 150, and a VL comprising an LCDR1 comprising the amino acid sequence of residues 24-38 of SEQ ID NO: 172, an LCDR2 comprising the amino acid sequence of residues 54-60 of SEQ ID NO: 172, and an LCDR3 comprising the amino acid sequence of residues 93-101 of SEQ ID NO: 172;
a VH comprising an HCDR1 comprising the amino acid sequence of positions 26-35 of SEQ ID NO: 152, an HCDR2 comprising the amino acid sequence of positions 50-66 of SEQ ID NO: 152, and an HCDR3 comprising the amino acid sequence of positions 99-108 of SEQ ID NO: 152, and a VL comprising an LCDR1 comprising the amino acid sequence of residues 24-38 of SEQ ID NO: 172, an LCDR2 comprising the amino acid sequence of residues 54-60 of SEQ ID NO: 172, and an LCDR3 comprising the amino acid sequence of residues 93-101 of SEQ ID NO: 172;
a VH comprising an HCDR1 comprising the amino acid sequence of positions 26-35 of SEQ ID NO: 154, an HCDR2 comprising the amino acid sequence of positions 50-66 of SEQ ID NO: 154, and an HCDR3 comprising the amino acid sequence of positions 99-108 of SEQ ID NO: 154, and a VL comprising an LCDR1 comprising the amino acid sequence of residues 24-38 of SEQ ID NO: 172, an LCDR2 comprising the amino acid sequence of residues 54-60 of SEQ ID NO: 172, and an LCDR3 comprising the amino acid sequence of residues 93-101 of SEQ ID NO: 172;
a VH comprising an HCDR1 comprising the amino acid sequence of positions 26-35 of SEQ ID NO: 156, an HCDR2 comprising the amino acid sequence of positions 50-66 of SEQ ID NO: 156, and an HCDR3 comprising the amino acid sequence of positions 99-108 of SEQ ID NO: 156, and a VL comprising an LCDR1 comprising the amino acid sequence of residues 24-38 of SEQ ID NO: 172, an LCDR2 comprising the amino acid sequence of residues 54-60 of SEQ ID NO: 172, and an LCDR3 comprising the amino acid sequence of residues 93-101 of SEQ ID NO: 172;
a VH comprising an HCDR1 comprising the amino acid sequence of positions 26-35 of SEQ ID NO: 158, an HCDR2 comprising the amino acid sequence of positions 50-66 of SEQ ID NO: 158, and an HCDR3 comprising the amino acid sequence of positions 99-108 of SEQ ID NO: 158, and a VL comprising an LCDR1 comprising the amino acid sequence of residues 24-38 of SEQ ID NO: 172, an LCDR2 comprising the amino acid sequence of residues 54-60 of SEQ ID NO: 172, and an LCDR3 comprising the amino acid sequence of residues 93-101 of SEQ ID NO: 172;
a VH comprising an HCDR1 comprising the amino acid sequence of positions 26-35 of SEQ ID NO: 160, an HCDR2 comprising the amino acid sequence of positions 50-66 of SEQ ID NO: 160, and an HCDR3 comprising the amino acid sequence of positions 99-108 of SEQ ID NO: 160, and a VL comprising an LCDR1 comprising the amino acid sequence of residues 24-38 of SEQ ID NO: 172, an LCDR2 comprising the amino acid sequence of residues 54-60 of SEQ ID NO: 172, and an LCDR3 comprising the amino acid sequence of residues 93-101 of SEQ ID NO: 172;
a VH comprising an HCDR1 comprising the amino acid sequence of positions 26-35 of SEQ ID NO: 162, an HCDR2 comprising the amino acid sequence of positions 50-66 of SEQ ID NO: 162, and an HCDR3 comprising the amino acid sequence of positions 99-108 of SEQ ID NO: 162, and a VL comprising an LCDR1 comprising the amino acid sequence of residues 24-38 of SEQ ID NO: 172, an LCDR2 comprising the amino acid sequence of residues 54-60 of SEQ ID NO: 172, and an LCDR3 comprising the amino acid sequence of residues 93-101 of SEQ ID NO: 172;
a VH comprising an HCDR1 comprising the amino acid sequence of positions 26-35 of SEQ ID NO: 164, an HCDR2 comprising the amino acid sequence of positions 50-66 of SEQ ID NO: 164, and an HCDR3 comprising the amino acid sequence of positions 99-108 of SEQ ID NO: 164, and a VL comprising an LCDR1 comprising the amino acid sequence of residues 24-38 of SEQ ID NO: 172, an LCDR2 comprising the amino acid sequence of residues 54-60 of SEQ ID NO: 172, and an LCDR3 comprising the amino acid sequence of residues 93-101 of SEQ ID NO: 172;
a VH comprising an HCDR1 comprising the amino acid sequence of positions 26-35 of SEQ ID NO: 166, an HCDR2 comprising the amino acid sequence of positions 50-66 of SEQ ID NO: 166, and an HCDR3 comprising the amino acid sequence of positions 99-108 of SEQ ID NO: 166, and a VL comprising an LCDR1 comprising the amino acid sequence of residues 24-38 of SEQ ID NO: 172, an LCDR2 comprising the amino acid sequence of residues 54-60 of SEQ ID NO: 172, and an LCDR3 comprising the amino acid sequence of residues 93-101 of SEQ ID NO: 172; or
a VH comprising an HCDR1 comprising the amino acid sequence of positions 26-35 of SEQ ID NO: 168, an HCDR2 comprising the amino acid sequence of positions 50-66 of SEQ ID NO: 168, and an HCDR3 comprising the amino acid sequence of positions 99-108 of SEQ ID NO: 168, and a VL comprising an LCDR1 comprising the amino acid sequence of residues 24-38 of SEQ ID NO: 172, an LCDR2 comprising the amino acid sequence of residues 54-60 of SEQ ID NO: 172, and an LCDR3 comprising the amino acid sequence of residues 93-101 of SEQ ID NO: 172.

In some embodiments above, the antibody comprises a VH comprising an amino acid sequence that is at least 90% identical to, at least 95% identical to, or at least 97% identical to the amino acid sequence of SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, or SEQ ID NO: 168. In some embodiments above, the antibody comprises a VL comprising an amino acid sequence that is at least 90% identical to, at least 95% identical to, or at least 97% identical to the amino acid sequence of SEQ ID NO: 170. In other embodiments, the antibody comprises a VL comprising an amino acid sequence that is at least 90% identical to, at least 95% identical to, or at least 97% identical to the amino acid sequence of SEQ ID NO: 172. In some embodiments, the antibody further comprises the corresponding HCDR1, HCDR2, HCDR3, LCDR1, LCDR2, and LCDR3 as provided above.

In some embodiments above, the antibody comprises a VH comprising the amino acid sequence of SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, or SEQ ID NO: 168. In some embodiments above, the antibody comprises a VL comprising the amino acid sequence of SEQ ID NO: 170. In some embodiments, the antibody comprises a VL comprising the amino acid sequence of SEQ ID NO: 172. In some embodiments above, the antibody comprises a VH comprising the amino acid sequence of SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, or SEQ ID NO: 168, and also a VL comprising the amino acid sequence of SEQ ID NO: 170. In some embodiments above, the antibody comprises a VH comprising the amino acid sequence of SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, or SEQ ID NO: 168, and also a VL comprising the amino acid sequence of SEQ ID NO: 172.

In some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of positions 26-35 of SEQ ID NO: 138, an HCDR2 comprising the amino acid sequence of positions 50-66 of SEQ ID NO: 138, and an HCDR3 comprising the amino acid sequence of positions 99-108 of SEQ ID NO: 138; and a light chain variable region (VL) comprising a light chain complementarity determining region 1 (LCDR1) comprising the amino acid sequence residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170. In some embodiments, the antibody comprises a VH that is at least 90%, at least 95%, at least 97%, or at least 99% identical to the amino acid sequence of SEQ ID NO: 138. In some embodiments, the antibody comprises a VL that is at least 90%, at least 95%, at least 97%, or at least 99% identical to the amino acid sequence of SEQ ID NO: 170. In some embodiments, the antibody comprises both a VH that is at least 90%, at least 95%, at least 97%, or at least 99% identical to the amino acid sequence of SEQ ID NO: 138; and a VL that is at least 90%, at least 95%, at least 97%, or at least 99% identical to the amino acid sequence of SEQ ID NO: 170. In some embodiments, the antibody comprises a VH comprising the amino acid sequence of SEQ ID No: 138 modified by 1-10 amino acid substitutions, 1-5 amino acid substitutions, or 1-3 amino acid substitutions. In some embodiments, the antibody comprises a VL comprising the amino acid sequence of SEQ ID No: 170 modified by 1-10 amino acid substitutions, 1-5 amino acid substitutions, or 1-3 amino acid substitutions. In some embodiments, the antibody comprises both a VH comprising the amino acid sequence of SEQ ID No: 138 modified by 1-10 amino acid substitutions, 1-5 amino acid substitutions, or 1-3 amino acid substitutions and a VL comprising the amino acid sequence of SEQ ID No: 170 modified by 1-10 amino acid substitutions, 1-5 amino acid substitutions, or 1-3 amino acid substitutions. In some embodiments, the antibody comprises a VL that is at least 90%, at least 95%, at least 97%, or at least 99% identical to the amino acid sequence of SEQ ID NO: 172. In some embodiments, the antibody comprises both a VH that is at least 90%, at least 95%, at least 97%, or at least 99% identical to the amino acid sequence of SEQ ID NO: 138; and a VL that is at least 90%, at least 95%, at least 97%, or at least 99% identical to the amino acid sequence of SEQ ID NO: 172. In some embodiments, the antibody comprises a VH comprising the amino acid sequence of SEQ ID No: 138 modified by 1-10 amino acid substitutions, 1-5 amino acid substitutions, or 1-3 amino acid substitutions. In some embodiments, the antibody comprises a VL comprising the amino acid sequence of SEQ ID No: 172 modified by 1-10 amino acid substitutions, 1-5 amino acid substitutions, or 1-3 amino acid substitutions. In some embodiments, the antibody comprises both a VH comprising the amino acid sequence of SEQ ID No: 138 modified by 1-10 amino acid substitutions, 1-5 amino acid substitutions, or 1-3 amino acid substitutions and a VL comprising the amino acid sequence of SEQ ID No: 172 modified by 1-10 amino acid substitutions, 1-5 amino acid substitutions, or 1-3 amino acid substitutions.

In some embodiments, the antibody comprises a VH comprising the amino acid sequence of SEQ ID No: 138. In some embodiments, the antibody comprises a VH comprising the amino acid sequence of SEQ ID No: 138; and a VL that is at least 90%, at least 95%, at least 97%, or at least 99% identical to the amino acid sequence of SEQ ID NO: 170. In some embodiments, the antibody comprises a VL comprising the amino acid sequence of SEQ ID No: 170. In some embodiments, the antibody comprises a VH that is at least 90%, at least 95%, at least 97%, or at least 99% identical to the amino acid sequence of SEQ ID NO: 138; and a VL comprising the amino acid sequence of SEQ ID No: 170. In some embodiments, the antibody comprises a VH comprising the amino acid sequence of SEQ ID No: 138 and a VL comprising the amino acid sequence of SEQ ID No: 170.

In some embodiments, the antibody comprises a VH comprising the amino acid sequence of SEQ ID No: 138. In some embodiments, the antibody comprises a VH comprising the amino acid sequence of SEQ ID No: 138; and a VL that is at least 90%, at least 95%, at least 97%, or at least 99% identical to the amino acid sequence of SEQ ID NO: 172. In some embodiments, the antibody comprises a VL comprising the amino acid sequence of SEQ ID No: 172. In some embodiments, the antibody comprises a VH that is at least 90%, at least 95%, at least 97%, or at least 99% identical to the amino acid sequence of SEQ ID NO: 138; and a VL comprising the amino acid sequence of SEQ ID No: 172. In some embodiments, the antibody comprises a VH comprising the amino acid sequence of SEQ ID No: 138 and a VL comprising the amino acid sequence of SEQ ID No: 172.

In some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of positions 26-35 of SEQ ID NO: 140, an HCDR2 comprising the amino acid sequence of positions 50-66 of SEQ ID NO: 140, and an HCDR3 comprising the amino acid sequence of positions 99-108 of SEQ ID NO: 140; and a light chain variable region (VL) comprising a light chain complementarity determining region 1 (LCDR1) comprising the amino acid sequence residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170. In some embodiments, the antibody comprises a VH that is at least 90%, at least 95%, at least 97%, or at least 99% identical to the amino acid sequence of SEQ ID NO: 140. In some embodiments, the antibody comprises a VL that is at least 90%, at least 95%, at least 97%, or the antibody comprises both a VH that is at least 90%, at least 95%, at least 97%, or at least 99% identical to the amino acid sequence of SEQ ID NO: 140; and a VL that is at least 90%, at least 95%, at least 97%, or at least 99% identical to the amino acid sequence of SEQ ID NO: 170. In some embodiments, the antibody comprises a VH comprising the amino acid sequence of SEQ ID No: 140 modified by 1-10 amino acid substitutions, 1-5 amino acid substitutions, or 1-3 amino acid substitutions. In some embodiments, the antibody comprises a VL comprising the amino acid sequence of SEQ ID No: 170 modified by 1-10 amino acid substitutions, 1-5 amino acid substitutions, or 1-3 amino acid substitutions. In some embodiments, the antibody comprises both a VH comprising the amino acid sequence of SEQ ID No: 140 modified by 1-10 amino acid substitutions, 1-5 amino acid substitutions, or 1-3 amino acid substitutions and a VL comprising the amino acid sequence of SEQ ID No: 170 modified by 1-10 amino acid substitutions, 1-5 amino acid substitutions, or 1-3 amino acid substitutions. In some embodiments, the antibody comprises a VL that is at least 90%, at least 95%, at least 97%, or at least 99% identical to the amino acid sequence of SEQ ID NO: 172. In some embodiments, the antibody comprises both a VH that is at least 90%, at least 95%, at least 97%, or at least 99% identical to the amino acid sequence of SEQ ID NO: 140; and a VL that is at least 90%, at least 95%, at least 97%, or at least 99% identical to the amino acid sequence of SEQ ID NO: 172. In some embodiments, the antibody comprises a VH comprising the amino acid sequence of SEQ ID No: 140 modified by 1-10 amino acid substitutions, 1-5 amino acid substitutions, or 1-3 amino acid substitutions. In some embodiments, the antibody comprises a VL comprising the amino acid sequence of SEQ ID No: 172 modified by 1-10 amino acid substitutions, 1-5 amino acid substitutions, or 1-3 amino acid substitutions. In some embodiments, the antibody comprises both a VH comprising the amino acid sequence of SEQ ID No: 140 modified by 1-10 amino acid substitutions, 1-5 amino acid substitutions, or 1-3 amino acid substitutions and a VL comprising the amino acid sequence of SEQ ID No: 172 modified by 1-10 amino acid substitutions, 1-5 amino acid substitutions, or 1-3 amino acid substitutions.

In some embodiments, the antibody comprises a VH comprising the amino acid sequence of SEQ ID No: 140. In some embodiments, the antibody comprises a VH comprising the amino acid sequence of SEQ ID No: 140; and a VL that is at least 90%, at least 95%, at least 97%, or at least 99% identical to the amino acid sequence of SEQ ID NO: 170. In some embodiments, the antibody comprises a VL comprising the amino acid sequence of SEQ ID No: 170. In some embodiments, the antibody comprises a VH that is at SEQ ID NO: 140; and a VL comprising the amino acid sequence of SEQ ID No: 170. In some embodiments, the antibody comprises a VH comprising the amino acid sequence of SEQ ID No: 140 and a VL comprising the amino acid sequence of SEQ ID No: 170.

In some embodiments, the antibody comprises a VH comprising the amino acid sequence of SEQ ID No: 140. In some embodiments, the antibody comprises a VH comprising the amino acid sequence of SEQ ID No: 140; and a VL that is at least 90%, at least 95%, at least 97%, or at least 99% identical to the amino acid sequence of SEQ ID NO: 172. In some embodiments, the antibody comprises a VL comprising the amino acid sequence of SEQ ID No: 172. In some embodiments, the antibody comprises a VH that is at least 90%, at least 95%, at least 97%, or at least 99% identical to the amino acid sequence of SEQ ID NO: 140; and a VL comprising the amino acid sequence of SEQ ID No: 172. In some embodiments, the antibody comprises a VH comprising the amino acid sequence of SEQ ID No: 140 and a VL comprising the amino acid sequence of SEQ ID No: 172.

In some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of positions 26-35 of SEQ ID NO: 142, an HCDR2 comprising the amino acid sequence of positions 50-66 of SEQ ID NO: 142, and an HCDR3 comprising the amino acid sequence of positions 99-108 of SEQ ID NO: 142; and a light chain variable region (VL) comprising a light chain complementarity determining region 1 (LCDR1) comprising the amino acid sequence residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170. In some embodiments, the antibody comprises a VH that is at least 90%, at least 95%, at least 97%, or at least 99% identical to the amino acid sequence of SEQ ID NO: 142. In some embodiments, the antibody comprises a VL that is at least 90%, at least 95%, at least 97%, or at least 99% identical to the amino acid sequence of SEQ ID NO: 170. In some embodiments, the antibody comprises both a VH that is at least 90%, at least 95%, at least 97%, or at least 99% identical to the amino acid sequence of SEQ ID NO: 142; and a VL that is at least 90%, at least 95%, at least 97%, or at least 99% identical to the amino acid sequence of SEQ ID NO: 170. In some embodiments, the antibody comprises a VH comprising the amino acid sequence of SEQ ID No: 142 modified by 1-10 amino acid substitutions, 1-5 amino acid substitutions, or 1-3 amino acid substitutions. In some embodiments, the antibody comprises substitutions, 1-5 amino acid substitutions, or 1-3 amino acid substitutions. In some embodiments, the antibody comprises both a VH comprising the amino acid sequence of SEQ ID No: 142 modified by 1-10 amino acid substitutions, 1-5 amino acid substitutions, or 1-3 amino acid substitutions and a VL comprising the amino acid sequence of SEQ ID No: 170 modified by 1-10 amino acid substitutions, 1-5 amino acid substitutions, or 1-3 amino acid substitutions. In some embodiments, the antibody comprises a VL that is at least 90%, at least 95%, at least 97%, or at least 99% identical to the amino acid sequence of SEQ ID NO: 172. In some embodiments, the antibody comprises both a VH that is at least 90%, at least 95%, at least 97%, or at least 99% identical to the amino acid sequence of SEQ ID NO: 142; and a VL that is at least 90%, at least 95%, at least 97%, or at least 99% identical to the amino acid sequence of SEQ ID NO: 172. In some embodiments, the antibody comprises a VH comprising the amino acid sequence of SEQ ID No: 142 modified by 1-10 amino acid substitutions, 1-5 amino acid substitutions, or 1-3 amino acid substitutions. In some embodiments, the antibody comprises a VL comprising the amino acid sequence of SEQ ID No: 172 modified by 1-10 amino acid substitutions, 1-5 amino acid substitutions, or 1-3 amino acid substitutions. In some embodiments, the antibody comprises both a VH comprising the amino acid sequence of SEQ ID No: 142 modified by 1-10 amino acid substitutions, 1-5 amino acid substitutions, or 1-3 amino acid substitutions and a VL comprising the amino acid sequence of SEQ ID No: 172 modified by 1-10 amino acid substitutions, 1-5 amino acid substitutions, or 1-3 amino acid substitutions.

In some embodiments, the antibody comprises a VH comprising the amino acid sequence of SEQ ID No: 142. In some embodiments, the antibody comprises a VH comprising the amino acid sequence of SEQ ID No: 142; and a VL that is at least 90%, at least 95%, at least 97%, or at least 99% identical to the amino acid sequence of SEQ ID NO: 170. In some embodiments, the antibody comprises a VL comprising the amino acid sequence of SEQ ID No: 170. In some embodiments, the antibody comprises a VH that is at least 90%, at least 95%, at least 97%, or at least 99% identical to the amino acid sequence of SEQ ID NO: 142; and a VL comprising the amino acid sequence of SEQ ID No: 170. In some embodiments, the antibody comprises a VH comprising the amino acid sequence of SEQ ID No: 142 and a VL comprising the amino acid sequence of SEQ ID No: 170.

In some embodiments, the antibody comprises a VH comprising the amino acid sequence of SEQ ID No: 142. In some embodiments, the antibody comprises a VH comprising the amino acid sequence of SEQ ID No: 142; and a VL that is at least 90%, at least 95%, at least 97%, or at least 99% identical to the amino acid sequence of SEQ ID NO: 172. In some embodiments, the antibody comprises a VL comprising the amino acid sequence of SEQ ID No: 172. In some embodiments, the antibody comprises a VH that is at least 90%, at least 95%, at least 97%, or at least 99% identical to the amino acid sequence of SEQ ID NO: 142; and a VL comprising the amino acid sequence of SEQ ID No: 172. In some embodiments, the antibody comprises a VH comprising the amino acid sequence of SEQ ID No: 142 and a VL comprising the amino acid sequence of SEQ ID No: 172.

In some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of positions 26-35 of SEQ ID NO: 144, an HCDR2 comprising the amino acid sequence of positions 50-66 of SEQ ID NO: 144, and an HCDR3 comprising the amino acid sequence of positions 99-108 of SEQ ID NO: 144; and a light chain variable region (VL) comprising a light chain complementarity determining region 1 (LCDR1) comprising the amino acid sequence residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170. In some embodiments, the antibody comprises a VH that is at least 90%, at least 95%, at least 97%, or at least 99% identical to the amino acid sequence of SEQ ID NO: 144. In some embodiments, the antibody comprises a VL that is at least 90%, at least 95%, at least 97%, or at least 99% identical to the amino acid sequence of SEQ ID NO: 170. In some embodiments, the antibody comprises both a VH that is at least 90%, at least 95%, at least 97%, or at least 99% identical to the amino acid sequence of SEQ ID NO: 144; and a VL that is at least 90%, at least 95%, at least 97%, or at least 99% identical to the amino acid sequence of SEQ ID NO: 170. In some embodiments, the antibody comprises a VH comprising the amino acid sequence of SEQ ID No: 144 modified by 1-10 amino acid substitutions, 1-5 amino acid substitutions, or 1-3 amino acid substitutions. In some embodiments, the antibody comprises a VL comprising the amino acid sequence of SEQ ID No: 170 modified by 1-10 amino acid substitutions, 1-5 amino acid substitutions, or 1-3 amino acid substitutions. In some embodiments, the antibody comprises both a VH comprising the amino acid sequence of SEQ ID No: 144 modified by 1-10 amino acid substitutions, 1-5 amino acid substitutions, or 1-3 amino acid substitutions and a VL comprising the amino acid sequence of SEQ ID No: 170 modified by 1-10 amino acid substitutions, 1-5 amino acid substitutions, or 1-3 amino acid substitutions. In some embodiments, the antibody comprises a VL that is at least 90%, at least In some embodiments, the antibody comprises both a VH that is at least 90%, at least 95%, at least 97%, or at least 99% identical to the amino acid sequence of SEQ ID NO: 144; and a VL that is at least 90%, at least 95%, at least 97%, or at least 99% Identical to the amino acid sequence of SEQ ID NO: 172. In some embodiments, the antibody comprises a VH comprising the amino acid sequence of SEQ ID No: 144 modified by 1-10 amino acid substitutions, 1-5 amino acid substitutions, or 1-3 amino acid substitutions. In some embodiments, the antibody comprises a VL comprising the amino acid sequence of SEQ ID No: 172 modified by 1-10 amino acid substitutions, 1-5 amino acid substitutions, or 1-3 amino acid substitutions. In some embodiments, the antibody comprises both a VH comprising the amino acid sequence of SEQ ID No: 144 modified by 1-10 amino acid substitutions, 1-5 amino acid substitutions, or 1-3 amino acid substitutions and a VL comprising the amino acid sequence of SEQ ID No: 172 modified by 1-10 amino acid substitutions, 1-5 amino acid substitutions, or 1-3 amino acid substitutions.

In some embodiments, the antibody comprises a VH comprising the amino acid sequence of SEQ ID No: 144. In some embodiments, the antibody comprises a VH comprising the amino acid sequence of SEQ ID No: 144; and a VL that is at least 90%, at least 95%, at least 97%, or at least 99% identical to the amino acid sequence of SEQ ID NO: 170. In some embodiments, the antibody comprises a VL comprising the amino acid sequence of SEQ ID No: 170. In some embodiments, the antibody comprises a VH that is at least 90%, at least 95%, at least 97%, or at least 99% identical to the amino acid sequence of SEQ ID NO: 144; and a VL comprising the amino acid sequence of SEQ ID No: 170. In some embodiments, the antibody comprises a VH comprising the amino acid sequence of SEQ ID No: 144 and a VL comprising the amino acid sequence of SEQ ID No: 170.

In some embodiments, the antibody comprises a VH comprising the amino acid sequence of SEQ ID No: 144. In some embodiments, the antibody comprises a VH comprising the amino acid sequence of SEQ ID No: 144; and a VL that is at least 90%, at least 95%, at least 97%, or at least 99% identical to the amino acid sequence of SEQ ID NO: 172. In some embodiments, the antibody comprises a VL comprising the amino acid sequence of SEQ ID No: 172. In some embodiments, the antibody comprises a VH that is at least 90%, at least 95%, at least 97%, or at least 99% identical to the amino acid sequence of SEQ ID NO: 144; and a VL comprising the amino acid sequence of SEQ ID No: 172. In some embodiments, the antibody comprises a VH comprising the amino acid sequence of SEQ ID No: 144 and a VL comprising the amino acid sequence of SEQ ID No: 172.

In some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of positions 26-35 of SEQ ID NO: 146, an HCDR2 comprising the amino acid sequence of positions 50-66 of SEQ ID NO: 146, and an HCDR3 comprising the amino acid sequence of positions 99-108 of SEQ ID NO: 146; and a light chain variable region (VL) comprising a light chain complementarity determining region 1 (LCDR1) comprising the amino acid sequence residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170. In some embodiments, the antibody comprises a VH that is at least 90%, at least 95%, at least 97%, or at least 99% identical to the amino acid sequence of SEQ ID NO: 146. In some embodiments, the antibody comprises a VL that is at least 90%, at least 95%, at least 97%, or at least 99% identical to the amino acid sequence of SEQ ID NO: 170. In some embodiments, the antibody comprises both a VH that is at least 90%, at least 95%, at least 97%, or at least 99% identical to the amino acid sequence of SEQ ID NO: 146; and a VL that is at least 90%, at least 95%, at least 97%, or at least 99% identical to the amino acid sequence of SEQ ID NO: 170. In some embodiments, the antibody comprises a VH comprising the amino acid sequence of SEQ ID No: 146 modified by 1-10 amino acid substitutions, 1-5 amino acid substitutions, or 1-3 amino acid substitutions. In some embodiments, the antibody comprises a VL comprising the amino acid sequence of SEQ ID No: 170 modified by 1-10 amino acid substitutions, 1-5 amino acid substitutions, or 1-3 amino acid substitutions. In some embodiments, the antibody comprises both a VH comprising the amino acid sequence of SEQ ID No: 146 modified by 1-10 amino acid substitutions, 1-5 amino acid substitutions, or 1-3 amino acid substitutions and a VL comprising the amino acid sequence of SEQ ID No: 170 modified by 1-10 amino acid substitutions, 1-5 amino acid substitutions, or 1-3 amino acid substitutions. In some embodiments, the antibody comprises a VL that is at least 90%, at least 95%, at least 97%, or at least 99% identical to the amino acid sequence of SEQ ID NO: 172. In some embodiments, the antibody comprises both a VH that is at least 90%, at least 95%, at least 97%, or at least 99% identical to the amino acid sequence of SEQ ID NO: 146; and a VL that is at least 90%, at least 95%, at least 97%, or at least 99% identical to the amino acid sequence of SEQ ID NO: 172. In some embodiments, the antibody comprises a VH comprising the amino acid sequence of SEQ ID No: 146 modified by 1-10 amino acid substitutions, 1-5 amino acid substitutions, or 1-3 amino acid substitutions. In some embodiments, the antibody comprises a VL comprising the amino acid sequence of SEQ ID No: 172 modified by 1-10 amino acid substitutions, 1-5 amino acid substitutions, or 1-3 amino acid substitutions. In some embodiments, the antibody comprises both a VH comprising the amino acid sequence of SEQ ID No: 146 modified by 1-10 amino acid substitutions, 1-5 amino acid substitutions, or 1-3 amino acid substitutions and a VL comprising the amino acid sequence of SEQ ID No: 172 modified by 1-10 amino acid substitutions, 1-5 amino acid substitutions, or 1-3 amino acid substitutions.

In some embodiments, the antibody comprises a VH comprising the amino acid sequence of SEQ ID No: 146. In some embodiments, the antibody comprises a VH comprising the amino acid sequence of SEQ ID No: 146; and a VL that is at least 90%, at least 95%, at least 97%, or at least 99% identical to the amino acid sequence of SEQ ID NO: 170. In some embodiments, the antibody comprises a VL comprising the amino acid sequence of SEQ ID No: 170. In some embodiments, the antibody comprises a VH that is at least 90%, at least 95%, at least 97%, or at least 99% identical to the amino acid sequence of SEQ ID NO: 146; and a VL comprising the amino acid sequence of SEQ ID No: 170. In some embodiments, the antibody comprises a VH comprising the amino acid sequence of SEQ ID No: 146 and a VL comprising the amino acid sequence of SEQ ID No: 170.

In some embodiments, the antibody comprises a VH comprising the amino acid sequence of SEQ ID No: 146. In some embodiments, the antibody comprises a VH comprising the amino acid sequence of SEQ ID No: 146; and a VL that is at least 90%, at least 95%, at least 97%, or at least 99% identical to the amino acid sequence of SEQ ID NO: 172. In some embodiments, the antibody comprises a VL comprising the amino acid sequence of SEQ ID No: 172. In some embodiments, the antibody comprises a VH that is at least 90%, at least 95%, at least 97%, or at least 99% identical to the amino acid sequence of SEQ ID NO: 146; and a VL comprising the amino acid sequence of SEQ ID No: 172. In some embodiments, the antibody comprises a VH comprising the amino acid sequence of SEQ ID No: 146 and a VL comprising the amino acid sequence of SEQ ID No: 172.

In some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of positions 26-35 of SEQ ID NO: 148, an HCDR2 comprising the amino acid sequence of positions 50-66 of SEQ ID NO: 148, and an HCDR3 comprising the amino acid sequence of positions 99-108 of SEQ ID NO: 148; and a light chain variable region (VL) comprising a light chain complementarity determining region 1 (LCDR1) comprising the amino acid sequence residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170. In some embodiments, the antibody comprises a VH that is at least 90%, at least 95%, at least 97%, or at least 99% identical to the amino acid sequence of SEQ ID NO: 148. In some embodiments, the antibody comprises a VL that is at least 90%, at least 95%, at least 97%, or at least 99% identical to the amino acid sequence of SEQ ID NO: 170. In some embodiments, the antibody comprises both a VH that is at least 90%, at least 95%, at least 97%, or at least 99% identical to the amino acid sequence of SEQ ID NO: 148; and a VL that is at least 90%, at least 95%, at least 97%, or at least 99% identical to the amino acid sequence of SEQ ID NO: 170. In some embodiments, the antibody comprises a VH comprising the amino acid sequence of SEQ ID No: 148 modified by 1-10 amino acid substitutions, 1-5 amino acid substitutions, or 1-3 amino acid substitutions. In some embodiments, the antibody comprises a VL comprising the amino acid sequence of SEQ ID No: 170 modified by 1-10 amino acid substitutions, 1-5 amino acid substitutions, or 1-3 amino acid substitutions. In some embodiments, the antibody comprises both a VH comprising the amino acid sequence of SEQ ID No: 148 modified by 1-10 amino acid substitutions, 1-5 amino acid substitutions, or 1-3 amino acid substitutions and a VL comprising the amino acid sequence of SEQ ID No: 170 modified by 1-10 amino acid substitutions, 1-5 amino acid substitutions, or 1-3 amino acid substitutions. In some embodiments, the antibody comprises a VL that is at least 90%, at least 95%, at least 97%, or at least 99% identical to the amino acid sequence of SEQ ID NO: 172. In some embodiments, the antibody comprises both a VH that is at least 90%, at least 95%, at least 97%, or at least 99% identical to the amino acid sequence of SEQ ID NO: 148; and a VL that is at least 90%, at least 95%, at least 97%, or at least 99% identical to the amino acid sequence of SEQ ID NO: 172. In some embodiments, the antibody comprises a VH comprising the amino acid sequence of SEQ ID No: 148 modified by 1-10 amino acid substitutions, 1-5 amino acid substitutions, or 1-3 amino acid substitutions. In some embodiments, the antibody comprises a VL comprising the amino acid sequence of SEQ ID No: 172 modified by 1-10 amino acid substitutions, 1-5 amino acid substitutions, or 1-3 amino acid substitutions. In some embodiments, the antibody comprises both a VH comprising the amino acid sequence of SEQ ID No: 148 modified by 1-10 amino acid substitutions, 1-5 amino acid substitutions, or 1-3 amino acid substitutions and a VL comprising the amino acid sequence of SEQ ID No: 172 modified by 1-10 amino acid substitutions, 1-5 amino acid substitutions, or 1-3 amino acid substitutions.

In some embodiments, the antibody comprises a VH comprising the amino acid sequence of SEQ ID No: 148. In some embodiments, the antibody comprises a VH comprising the amino acid sequence of SEQ ID No: 148; and a VL that is at least 90%, at least 95%, at least 97%, or at least 99% identical to the amino acid sequence of SEQ ID NO: 170. In some embodiments, the antibody comprises a VL comprising the amino acid sequence of SEQ ID No: 170. In some embodiments, the antibody comprises a VH that is at least 90%, at least 95%, at least 97%, or at least 99% identical to the amino acid sequence of SEQ ID NO: 148; and a VL comprising the amino acid sequence of SEQ ID No: 170. In some embodiments, the antibody comprises a VH comprising the amino acid sequence of SEQ ID No: 148 and a VL comprising the amino acid sequence of SEQ ID No: 170.

In some embodiments, the antibody comprises a VH comprising the amino acid sequence of SEQ ID No: 148. In some embodiments, the antibody comprises a VH comprising the amino acid sequence of SEQ ID No: 148; and a VL that is at least 90%, at least 95%, at least 97%, or at least 99% identical to the amino acid sequence of SEQ ID NO: 172. In some embodiments, the antibody comprises a VL comprising the amino acid sequence of SEQ ID No: 172. In some embodiments, the antibody comprises a VH that is at least 90%, at least 95%, at least 97%, or at least 99% identical to the amino acid sequence of SEQ ID NO: 148; and a VL comprising the amino acid sequence of SEQ ID No: 172. In some embodiments, the antibody comprises a VH comprising the amino acid sequence of SEQ ID No: 148 and a VL comprising the amino acid sequence of SEQ ID No: 172.

In some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of positions 26-35 of SEQ ID NO: 150, an HCDR2 comprising the amino acid sequence of positions 50-66 of SEQ ID NO: 150, and an HCDR3 comprising the amino acid sequence of positions 99-108 of SEQ ID NO: 150; and a light chain variable region (VL) comprising a light chain complementarity determining region 1 (LCDR1) comprising the amino acid sequence residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170. In some embodiments, the antibody comprises a VH that is at least 90%, at least 95%, at least 97%, or at least 99% identical to the amino acid sequence of SEQ ID NO: 150. In some embodiments, the antibody comprises a VL that is at least 90%, at least 95%, at least 97%, or the antibody comprises both a VH that is at least 90%, at least 95%, at least 97%, or at least 99% identical to the amino acid sequence of SEQ ID NO: 150; and a VL that is at least 90%, at least 95%, at least 97%, or at least 99% identical to the amino acid sequence of SEQ ID NO: 170. In some embodiments, the antibody comprises a VH comprising the amino acid sequence of SEQ ID No: 150 modified by 1-10 amino acid substitutions, 1-5 amino acid substitutions, or 1-3 amino acid substitutions. In some embodiments, the antibody comprises a VL comprising the amino acid sequence of SEQ ID No: 170 modified by 1-10 amino acid substitutions, 1-5 amino acid substitutions, or 1-3 amino acid substitutions. In some embodiments, the antibody comprises both a VH comprising the amino acid sequence of SEQ ID No: 150 modified by 1-10 amino acid substitutions, 1-5 amino acid substitutions, or 1-3 amino acid substitutions and a VL comprising the amino acid sequence of SEQ ID No: 170 modified by 1-10 amino acid substitutions, 1-5 amino acid substitutions, or 1-3 amino acid substitutions. In some embodiments, the antibody comprises a VL that is at least 90%, at least 95%, at least 97%, or at least 99% identical to the amino acid sequence of SEQ ID NO: 172. In some embodiments, the antibody comprises both a VH that is at least 90%, at least 95%, at least 97%, or at least 99% identical to the amino acid sequence of SEQ ID NO: 150; and a VL that is at least 90%, at least 95%, at least 97%, or at least 99% identical to the amino acid sequence of SEQ ID NO: 172. In some embodiments, the antibody comprises a VH comprising the amino acid sequence of SEQ ID No: 150 modified by 1-10 amino acid substitutions, 1-5 amino acid substitutions, or 1-3 amino acid substitutions. In some embodiments, the antibody comprises a VL comprising the amino acid sequence of SEQ ID No: 172 modified by 1-10 amino acid substitutions, 1-5 amino acid substitutions, or 1-3 amino acid substitutions. In some embodiments, the antibody comprises both a VH comprising the amino acid sequence of SEQ ID No: 150 modified by 1-10 amino acid substitutions, 1-5 amino acid substitutions, or 1-3 amino acid substitutions and a VL comprising the amino acid sequence of SEQ ID No: 172 modified by 1-10 amino acid substitutions, 1-5 amino acid substitutions, or 1-3 amino acid substitutions.

In some embodiments, the antibody comprises a VH comprising the amino acid sequence of SEQ ID No: 150. In some embodiments, the antibody comprises a VH comprising the amino acid sequence of SEQ ID No: 150; and a VL that is at least 90%, at least 95%, at least 97%, or at least 99% identical to the amino acid sequence of SEQ ID NO: 170. In some embodiments, the antibody comprises a VL comprising the amino acid sequence of SEQ ID No: 170. In some embodiments, the antibody comprises a VH that is at SEQ ID NO: 150; and a VL comprising the amino acid sequence of SEQ ID No: 170. In some embodiments, the antibody comprises a VH comprising the amino acid sequence of SEQ ID No: 150 and a VL comprising the amino acid sequence of SEQ ID No: 170.

In some embodiments, the antibody comprises a VH comprising the amino acid sequence of SEQ ID No: 150. In some embodiments, the antibody comprises a VH comprising the amino acid sequence of SEQ ID No: 150; and a VL that is at least 90%, at least 95%, at least 97%, or at least 99% identical to the amino acid sequence of SEQ ID NO: 172. In some embodiments, the antibody comprises a VL comprising the amino acid sequence of SEQ ID No: 172. In some embodiments, the antibody comprises a VH that is at least 90%, at least 95%, at least 97%, or at least 99% identical to the amino acid sequence of SEQ ID NO: 150; and a VL comprising the amino acid sequence of SEQ ID No: 172. In some embodiments, the antibody comprises a VH comprising the amino acid sequence of SEQ ID No: 150 and a VL comprising the amino acid sequence of SEQ ID No: 172.

In some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of positions 26-35 of SEQ ID NO: 152, an HCDR2 comprising the amino acid sequence of positions 50-66 of SEQ ID NO: 152, and an HCDR3 comprising the amino acid sequence of positions 99-108 of SEQ ID NO: 152; and a light chain variable region (VL) comprising a light chain complementarity determining region 1 (LCDR1) comprising the amino acid sequence residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170. In some embodiments, the antibody comprises a VH that is at least 90%, at least 95%, at least 97%, or at least 99% identical to the amino acid sequence of SEQ ID NO: 152. In some embodiments, the antibody comprises a VL that is at least 90%, at least 95%, at least 97%, or at least 99% identical to the amino acid sequence of SEQ ID NO: 170. In some embodiments, the antibody comprises both a VH that is at least 90%, at least 95%, at least 97%, or at least 99% identical to the amino acid sequence of SEQ ID NO: 152; and a VL that is at least 90%, at least 95%, at least 97%, or at least 99% identical to the amino acid sequence of SEQ ID NO: 170. In some embodiments, the antibody comprises a VH comprising the amino acid sequence of SEQ ID No: 152 modified by 1-10 amino acid substitutions, 1-5 amino acid substitutions, or 1-3 amino acid substitutions. In some embodiments, the antibody comprises substitutions, 1-5 amino acid substitutions, or 1-3 amino acid substitutions. In some embodiments, the antibody comprises both a VH comprising the amino acid sequence of SEQ ID No: 152 modified by 1-10 amino acid substitutions, 1-5 amino acid substitutions, or 1-3 amino acid substitutions and a VL comprising the amino acid sequence of SEQ ID No: 170 modified by 1-10 amino acid substitutions, 1-5 amino acid substitutions, or 1-3 amino acid substitutions. In some embodiments, the antibody comprises a VL that is at least 90%, at least 95%, at least 97%, or at least 99% identical to the amino acid sequence of SEQ ID NO: 172. In some embodiments, the antibody comprises both a VH that is at least 90%, at least 95%, at least 97%, or at least 99% identical to the amino acid sequence of SEQ ID NO: 152; and a VL that is at least 90%, at least 95%, at least 97%, or at least 99% identical to the amino acid sequence of SEQ ID NO: 172. In some embodiments, the antibody comprises a VH comprising the amino acid sequence of SEQ ID No: 152 modified by 1-10 amino acid substitutions, 1-5 amino acid substitutions, or 1-3 amino acid substitutions. In some embodiments, the antibody comprises a VL comprising the amino acid sequence of SEQ ID No: 172 modified by 1-10 amino acid substitutions, 1-5 amino acid substitutions, or 1-3 amino acid substitutions. In some embodiments, the antibody comprises both a VH comprising the amino acid sequence of SEQ ID No: 152 modified by 1-10 amino acid substitutions, 1-5 amino acid substitutions, or 1-3 amino acid substitutions and a VL comprising the amino acid sequence of SEQ ID No: 172 modified by 1-10 amino acid substitutions, 1-5 amino acid substitutions, or 1-3 amino acid substitutions.

In some embodiments, the antibody comprises a VH comprising the amino acid sequence of SEQ ID No: 152. In some embodiments, the antibody comprises a VH comprising the amino acid sequence of SEQ ID No: 152; and a VL that is at least 90%, at least 95%, at least 97%, or at least 99% identical to the amino acid sequence of SEQ ID NO: 170. In some embodiments, the antibody comprises a VL comprising the amino acid sequence of SEQ ID No: 170. In some embodiments, the antibody comprises a VH that is at least 90%, at least 95%, at least 97%, or at least 99% identical to the amino acid sequence of SEQ ID NO: 152; and a VL comprising the amino acid sequence of SEQ ID No: 170. In some embodiments, the antibody comprises a VH comprising the amino acid sequence of SEQ ID No: 152 and a VL comprising the amino acid sequence of SEQ ID No: 170.

In some embodiments, the antibody comprises a VH comprising the amino acid sequence of SEQ ID No: 152. In some embodiments, the antibody comprises a VH comprising the amino acid sequence of SEQ ID No: 152; and a VL that is at least 90%, at least 95%, at least 97%, or at least 99% identical to the amino acid sequence of SEQ ID NO: 172. In some embodiments, the antibody comprises a VL comprising the amino acid sequence of SEQ ID No: 172. In some embodiments, the antibody comprises a VH that is at least 90%, at least 95%, at least 97%, or at least 99% identical to the amino acid sequence of SEQ ID NO: 152; and a VL comprising the amino acid sequence of SEQ ID No: 172. In some embodiments, the antibody comprises a VH comprising the amino acid sequence of SEQ ID No: 152 and a VL comprising the amino acid sequence of SEQ ID No: 172.

In some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of positions 26-35 of SEQ ID NO: 154, an HCDR2 comprising the amino acid sequence of positions 50-66 of SEQ ID NO: 154, and an HCDR3 comprising the amino acid sequence of positions 99-108 of SEQ ID NO: 154; and a light chain variable region (VL) comprising a light chain complementarity determining region 1 (LCDR1) comprising the amino acid sequence residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170. In some embodiments, the antibody comprises a VH that is at least 90%, at least 95%, at least 97%, or at least 99% identical to the amino acid sequence of SEQ ID NO: 154. In some embodiments, the antibody comprises a VL that is at least 90%, at least 95%, at least 97%, or at least 99% identical to the amino acid sequence of SEQ ID NO: 170. In some embodiments, the antibody comprises both a VH that is at least 90%, at least 95%, at least 97%, or at least 99% identical to the amino acid sequence of SEQ ID NO: 154; and a VL that is at least 90%, at least 95%, at least 97%, or at least 99% identical to the amino acid sequence of SEQ ID NO: 170. In some embodiments, the antibody comprises a VH comprising the amino acid sequence of SEQ ID No: 154 modified by 1-10 amino acid substitutions, 1-5 amino acid substitutions, or 1-3 amino acid substitutions. In some embodiments, the antibody comprises a VL comprising the amino acid sequence of SEQ ID No: 170 modified by 1-10 amino acid substitutions, 1-5 amino acid substitutions, or 1-3 amino acid substitutions. In some embodiments, the antibody comprises both a VH comprising the amino acid sequence of SEQ ID No: 154 modified by 1-10 amino acid substitutions, 1-5 amino acid substitutions, or 1-3 amino acid substitutions and a VL comprising the amino acid sequence of SEQ ID No: 170 modified by 1-10 amino acid substitutions, 1-5 amino acid substitutions, or 1-3 amino acid substitutions. In some embodiments, the antibody comprises a VL that is at least 90%, at least In some embodiments, the antibody comprises both a VH that is at least 90%, at least 95%, at least 97%, or at least 99% identical to the amino acid sequence of SEQ ID NO: 154; and a VL that is at least 90%, at least 95%, at least 97%, or at least 99% identical to the amino acid sequence of SEQ ID NO: 172. In some embodiments, the antibody comprises a VH comprising the amino acid sequence of SEQ ID No: 154 modified by 1-10 amino acid substitutions, 1-5 amino acid substitutions, or 1-3 amino acid substitutions. In some embodiments, the antibody comprises a VL comprising the amino acid sequence of SEQ ID No: 172 modified by 1-10 amino acid substitutions, 1-5 amino acid substitutions, or 1-3 amino acid substitutions. In some embodiments, the antibody comprises both a VH comprising the amino acid sequence of SEQ ID No: 154 modified by 1-10 amino acid substitutions, 1-5 amino acid substitutions, or 1-3 amino acid substitutions and a VL comprising the amino acid sequence of SEQ ID No: 172 modified by 1-10 amino acid substitutions, 1-5 amino acid substitutions, or 1-3 amino acid substitutions.

In some embodiments, the antibody comprises a VH comprising the amino acid sequence of SEQ ID No: 154. In some embodiments, the antibody comprises a VH comprising the amino acid sequence of SEQ ID No: 154; and a VL that is at least 90%, at least 95%, at least 97%, or at least 99% identical to the amino acid sequence of SEQ ID NO: 170. In some embodiments, the antibody comprises a VL comprising the amino acid sequence of SEQ ID No: 170. In some embodiments, the antibody comprises a VH that is at least 90%, at least 95%, at least 97%, or at least 99% identical to the amino acid sequence of SEQ ID NO: 154; and a VL comprising the amino acid sequence of SEQ ID No: 170. In some embodiments, the antibody comprises a VH comprising the amino acid sequence of SEQ ID No: 154 and a VL comprising the amino acid sequence of SEQ ID No: 170.

In some embodiments, the antibody comprises a VH comprising the amino acid sequence of SEQ ID No: 154. In some embodiments, the antibody comprises a VH comprising the amino acid sequence of SEQ ID No: 154; and a VL that is at least 90%, at least 95%, at least 97%, or at least 99% identical to the amino acid sequence of SEQ ID NO: 172. In some embodiments, the antibody comprises a VL comprising the amino acid sequence of SEQ ID No: 172. In some embodiments, the antibody comprises a VH that is at least 90%, at least 95%, at least 97%, or at least 99% identical to the amino acid sequence of SEQ ID NO: 154; and a VL comprising the amino acid sequence of SEQ ID No: 172. In some embodiments, the antibody comprises a VH comprising the amino acid sequence of SEQ ID No: 154 and a VL comprising the amino acid sequence of SEQ ID No: 172.

In some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of positions 26-35 of SEQ ID NO: 156, an HCDR2 comprising the amino acid sequence of positions 50-66 of SEQ ID NO: 156, and an HCDR3 comprising the amino acid sequence of positions 99-108 of SEQ ID NO: 156; and a light chain variable region (VL) comprising a light chain complementarity determining region 1 (LCDR1) comprising the amino acid sequence residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170. In some embodiments, the antibody comprises a VH that is at least 90%, at least 95%, at least 97%, or at least 99% identical to the amino acid sequence of SEQ ID NO: 156. In some embodiments, the antibody comprises a VL that is at least 90%, at least 95%, at least 97%, or at least 99% identical to the amino acid sequence of SEQ ID NO: 170. In some embodiments, the antibody comprises both a VH that is at least 90%, at least 95%, at least 97%, or at least 99% identical to the amino acid sequence of SEQ ID NO: 156; and a VL that is at least 90%, at least 95%, at least 97%, or at least 99% identical to the amino acid sequence of SEQ ID NO: 170. In some embodiments, the antibody comprises a VH comprising the amino acid sequence of SEQ ID No: 156 modified by 1-10 amino acid substitutions, 1-5 amino acid substitutions, or 1-3 amino acid substitutions. In some embodiments, the antibody comprises a VL comprising the amino acid sequence of SEQ ID No: 170 modified by 1-10 amino acid substitutions, 1-5 amino acid substitutions, or 1-3 amino acid substitutions. In some embodiments, the antibody comprises both a VH comprising the amino acid sequence of SEQ ID No: 156 modified by 1-10 amino acid substitutions, 1-5 amino acid substitutions, or 1-3 amino acid substitutions and a VL comprising the amino acid sequence of SEQ ID No: 170 modified by 1-10 amino acid substitutions, 1-5 amino acid substitutions, or 1-3 amino acid substitutions. In some embodiments, the antibody comprises a VL that is at least 90%, at least 95%, at least 97%, or at least 99% identical to the amino acid sequence of SEQ ID NO: 172. In some embodiments, the antibody comprises both a VH that is at least 90%, at least 95%, at least 97%, or at least 99% identical to the amino acid sequence of SEQ ID NO: 156; and a VL that is at least 90%, at least 95%, at least 97%, or at least 99% identical to the amino acid sequence of SEQ ID NO: 172. In some embodiments, the antibody comprises a VH comprising the amino acid sequence of SEQ ID No: 156 modified by 1-10 amino acid substitutions, 1-5 amino acid substitutions, or 1-3 amino acid substitutions. In some embodiments, the antibody comprises a VL comprising the amino acid sequence of SEQ ID No: 172 modified by 1-10 amino acid substitutions, 1-5 amino acid substitutions, or 1-3 amino acid substitutions. In some embodiments, the antibody comprises both a VH comprising the amino acid sequence of SEQ ID No: 156 modified by 1-10 amino acid substitutions, 1-5 amino acid substitutions, or 1-3 amino acid substitutions and a VL comprising the amino acid sequence of SEQ ID No: 172 modified by 1-10 amino acid substitutions, 1-5 amino acid substitutions, or 1-3 amino acid substitutions.

In some embodiments, the antibody comprises a VH comprising the amino acid sequence of SEQ ID No: 156. In some embodiments, the antibody comprises a VH comprising the amino acid sequence of SEQ ID No: 156; and a VL that is at least 90%, at least 95%, at least 97%, or at least 99% identical to the amino acid sequence of SEQ ID NO: 170. In some embodiments, the antibody comprises a VL comprising the amino acid sequence of SEQ ID No: 170. In some embodiments, the antibody comprises a VH that is at least 90%, at least 95%, at least 97%, or at least 99% identical to the amino acid sequence of SEQ ID NO: 156; and a VL comprising the amino acid sequence of SEQ ID No: 170. In some embodiments, the antibody comprises a VH comprising the amino acid sequence of SEQ ID No: 156 and a VL comprising the amino acid sequence of SEQ ID No: 170.

In some embodiments, the antibody comprises a VH comprising the amino acid sequence of SEQ ID No: 156. In some embodiments, the antibody comprises a VH comprising the amino acid sequence of SEQ ID No: 156; and a VL that is at least 90%, at least 95%, at least 97%, or at least 99% identical to the amino acid sequence of SEQ ID NO: 172. In some embodiments, the antibody comprises a VL comprising the amino acid sequence of SEQ ID No: 172. In some embodiments, the antibody comprises a VH that is at least 90%, at least 95%, at least 97%, or at least 99% identical to the amino acid sequence of SEQ ID NO: 156; and a VL comprising the amino acid sequence of SEQ ID No: 172. In some embodiments, the antibody comprises a VH comprising the amino acid sequence of SEQ ID No: 156 and a VL comprising the amino acid sequence of SEQ ID No: 172.

In some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of positions 26-35 of SEQ ID NO: 158, an HCDR2 comprising the amino acid sequence of positions 50-66 of SEQ ID NO: 158, and an HCDR3 comprising the amino acid sequence of positions 99-108 of SEQ ID NO: 158; and a light chain variable region (VL) comprising a light chain complementarity determining region 1 (LCDR1) comprising the amino acid sequence residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170. In some embodiments, the antibody comprises a VH that is at least 90%, at least 95%, at least 97%, or at least 99% identical to the amino acid sequence of SEQ ID NO: 158. In some embodiments, the antibody comprises a VL that is at least 90%, at least 95%, at least 97%, or at least 99% identical to the amino acid sequence of SEQ ID NO: 170. In some embodiments, the antibody comprises both a VH that is at least 90%, at least 95%, at least 97%, or at least 99% identical to the amino acid sequence of SEQ ID NO: 158; and a VL that is at least 90%, at least 95%, at least 97%, or at least 99% identical to the amino acid sequence of SEQ ID NO: 170. In some embodiments, the antibody comprises a VH comprising the amino acid sequence of SEQ ID No: 158 modified by 1-10 amino acid substitutions, 1-5 amino acid substitutions, or 1-3 amino acid substitutions. In some embodiments, the antibody comprises a VL comprising the amino acid sequence of SEQ ID No: 170 modified by 1-10 amino acid substitutions, 1-5 amino acid substitutions, or 1-3 amino acid substitutions. In some embodiments, the antibody comprises both a VH comprising the amino acid sequence of SEQ ID No: 158 modified by 1-10 amino acid substitutions, 1-5 amino acid substitutions, or 1-3 amino acid substitutions and a VL comprising the amino acid sequence of SEQ ID No: 170 modified by 1-10 amino acid substitutions, 1-5 amino acid substitutions, or 1-3 amino acid substitutions. In some embodiments, the antibody comprises a VL that is at least 90%, at least 95%, at least 97%, or at least 99% identical to the amino acid sequence of SEQ ID NO: 172. In some embodiments, the antibody comprises both a VH that is at least 90%, at least 95%, at least 97%, or at least 99% identical to the amino acid sequence of SEQ ID NO: 158; and a VL that is at least 90%, at least 95%, at least 97%, or at least 99% identical to the amino acid sequence of SEQ ID NO: 172. In some embodiments, the antibody comprises a VH comprising the amino acid sequence of SEQ ID No: 158 modified by 1-10 amino acid substitutions, 1-5 amino acid substitutions, or 1-3 amino acid substitutions. In some embodiments, the antibody comprises a VL comprising the amino acid sequence of SEQ ID No: 172 modified by 1-10 amino acid substitutions, 1-5 amino acid substitutions, or 1-3 amino acid substitutions. In some embodiments, the antibody comprises both a VH comprising the amino acid sequence of SEQ ID No: 158 modified by 1-10 amino acid substitutions, 1-5 amino acid substitutions, or 1-3 amino acid substitutions and a VL comprising the amino acid sequence of SEQ ID No: 172 modified by 1-10 amino acid substitutions, 1-5 amino acid substitutions, or 1-3 amino acid substitutions.

In some embodiments, the antibody comprises a VH comprising the amino acid sequence of SEQ ID No: 158. In some embodiments, the antibody comprises a VH comprising the amino acid sequence of SEQ ID No: 158; and a VL that is at least 90%, at least 95%, at least 97%, or at least 99% identical to the amino acid sequence of SEQ ID NO: 170. In some embodiments, the antibody comprises a VL comprising the amino acid sequence of SEQ ID No: 170. In some embodiments, the antibody comprises a VH that is at least 90%, at least 95%, at least 97%, or at least 99% identical to the amino acid sequence of SEQ ID NO: 158; and a VL comprising the amino acid sequence of SEQ ID No: 170. In some embodiments, the antibody comprises a VH comprising the amino acid sequence of SEQ ID No: 158 and a VL comprising the amino acid sequence of SEQ ID No: 170.

In some embodiments, the antibody comprises a VH comprising the amino acid sequence of SEQ ID No: 158. In some embodiments, the antibody comprises a VH comprising the amino acid sequence of SEQ ID No: 158; and a VL that is at least 90%, at least 95%, at least 97%, or at least 99% identical to the amino acid sequence of SEQ ID NO: 172. In some embodiments, the antibody comprises a VL comprising the amino acid sequence of SEQ ID No: 172. In some embodiments, the antibody comprises a VH that is at least 90%, at least 95%, at least 97%, or at least 99% identical to the amino acid sequence of SEQ ID NO: 158; and a VL comprising the amino acid sequence of SEQ ID No: 172. In some embodiments, the antibody comprises a VH comprising the amino acid sequence of SEQ ID No: 158 and a VL comprising the amino acid sequence of SEQ ID No: 172.

In some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of positions 26-35 of SEQ ID NO: 160, an HCDR2 comprising the amino acid sequence of positions 50-66 of SEQ ID NO: 160, and an HCDR3 comprising the amino acid sequence of positions 99-108 of SEQ ID NO: 160; and a light chain variable region (VL) comprising a light chain complementarity determining region 1 (LCDR1) comprising the amino acid sequence residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170. In some embodiments, the antibody comprises a VH that is at least 90%, at least 95%, at least 97%, or at least 99% identical to the amino acid sequence of SEQ ID NO: 160. In some embodiments, the antibody comprises a VL that is at least 90%, at least 95%, at least 97%, or the antibody comprises both a VH that is at least 90%, at least 95%, at least 97%, or at least 99% identical to the amino acid sequence of SEQ ID NO: 160; and a VL that is at least 90%, at least 95%, at least 97%, or at least 99% identical to the amino acid sequence of SEQ ID NO: 170. In some embodiments, the antibody comprises a VH comprising the amino acid sequence of SEQ ID No: 160 modified by 1-10 amino acid substitutions, 1-5 amino acid substitutions, or 1-3 amino acid substitutions. In some embodiments, the antibody comprises a VL comprising the amino acid sequence of SEQ ID No: 170 modified by 1-10 amino acid substitutions, 1-5 amino acid substitutions, or 1-3 amino acid substitutions. In some embodiments, the antibody comprises both a VH comprising the amino acid sequence of SEQ ID No: 160 modified by 1-10 amino acid substitutions, 1-5 amino acid substitutions, or 1-3 amino acid substitutions and a VL comprising the amino acid sequence of SEQ ID No: 170 modified by 1-10 amino acid substitutions, 1-5 amino acid substitutions, or 1-3 amino acid substitutions. In some embodiments, the antibody comprises a VL that is at least 90%, at least 95%, at least 97%, or at least 99% identical to the amino acid sequence of SEQ ID NO: 172. In some embodiments, the antibody comprises both a VH that is at least 90%, at least 95%, at least 97%, or at least 99% identical to the amino acid sequence of SEQ ID NO: 160; and a VL that is at least 90%, at least 95%, at least 97%, or at least 99% identical to the amino acid sequence of SEQ ID NO: 172. In some embodiments, the antibody comprises a VH comprising the amino acid sequence of SEQ ID No: 160 modified by 1-10 amino acid substitutions, 1-5 amino acid substitutions, or 1-3 amino acid substitutions. In some embodiments, the antibody comprises a VL comprising the amino acid sequence of SEQ ID No: 172 modified by 1-10 amino acid substitutions, 1-5 amino acid substitutions, or 1-3 amino acid substitutions. In some embodiments, the antibody comprises both a VH comprising the amino acid sequence of SEQ ID No: 160 modified by 1-10 amino acid substitutions, 1-5 amino acid substitutions, or 1-3 amino acid substitutions and a VL comprising the amino acid sequence of SEQ ID No: 172 modified by 1-10 amino acid substitutions, 1-5 amino acid substitutions, or 1-3 amino acid substitutions.

In some embodiments, the antibody comprises a VH comprising the amino acid sequence of SEQ ID No: 160. In some embodiments, the antibody comprises a VH comprising the amino acid sequence of SEQ ID No: 160; and a VL that is at least 90%, at least 95%, at least 97%, or at least 99% identical to the amino acid sequence of SEQ ID NO: 170. In some embodiments, the antibody comprises a VL comprising the amino acid sequence of SEQ ID No: 170. In some embodiments, the antibody comprises a VH that is at SEQ ID NO: 160; and a VL comprising the amino acid sequence of SEQ ID No: 170. In some embodiments, the antibody comprises a VH comprising the amino acid sequence of SEQ ID No: 160 and a VL comprising the amino acid sequence of SEQ ID No: 170.

In some embodiments, the antibody comprises a VH comprising the amino acid sequence of SEQ ID No: 160. In some embodiments, the antibody comprises a VH comprising the amino acid sequence of SEQ ID No: 160; and a VL that is at least 90%, at least 95%, at least 97%, or at least 99% identical to the amino acid sequence of SEQ ID NO: 172. In some embodiments, the antibody comprises a VL comprising the amino acid sequence of SEQ ID No: 172. In some embodiments, the antibody comprises a VH that is at least 90%, at least 95%, at least 97%, or at least 99% identical to the amino acid sequence of SEQ ID NO: 160; and a VL comprising the amino acid sequence of SEQ ID No: 172. In some embodiments, the antibody comprises a VH comprising the amino acid sequence of SEQ ID No: 160 and a VL comprising the amino acid sequence of SEQ ID No: 172.

In some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of positions 26-35 of SEQ ID NO: 162, an HCDR2 comprising the amino acid sequence of positions 50-66 of SEQ ID NO: 162, and an HCDR3 comprising the amino acid sequence of positions 99-108 of SEQ ID NO: 162; and a light chain variable region (VL) comprising a light chain complementarity determining region 1 (LCDR1) comprising the amino acid sequence residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170. In some embodiments, the antibody comprises a VH that is at least 90%, at least 95%, at least 97%, or at least 99% identical to the amino acid sequence of SEQ ID NO: 162. In some embodiments, the antibody comprises a VL that is at least 90%, at least 95%, at least 97%, or at least 99% identical to the amino acid sequence of SEQ ID NO: 170. In some embodiments, the antibody comprises both a VH that is at least 90%, at least 95%, at least 97%, or at least 99% identical to the amino acid sequence of SEQ ID NO: 162; and a VL that is at least 90%, at least 95%, at least 97%, or at least 99% identical to the amino acid sequence of SEQ ID NO: 170. In some embodiments, the antibody comprises a VH comprising the amino acid sequence of SEQ ID No: 162 modified by 1-10 amino acid substitutions, 1-5 amino acid substitutions, or 1-3 amino acid substitutions. In some embodiments, the antibody comprises substitutions, 1-5 amino acid substitutions, or 1-3 amino acid substitutions. In some embodiments, the antibody comprises both a VH comprising the amino acid sequence of SEQ ID No: 162 modified by 1-10 amino acid substitutions, 1-5 amino acid substitutions, or 1-3 amino acid substitutions and a VL comprising the amino acid sequence of SEQ ID No: 170 modified by 1-10 amino acid substitutions, 1-5 amino acid substitutions, or 1-3 amino acid substitutions. In some embodiments, the antibody comprises a VL that is at least 90%, at least 95%, at least 97%, or at least 99% identical to the amino acid sequence of SEQ ID NO: 172. In some embodiments, the antibody comprises both a VH that is at least 90%, at least 95%, at least 97%, or at least 99% identical to the amino acid sequence of SEQ ID NO: 162; and a VL that is at least 90%, at least 95%, at least 97%, or at least 99% identical to the amino acid sequence of SEQ ID NO: 172. In some embodiments, the antibody comprises a VH comprising the amino acid sequence of SEQ ID No: 162 modified by 1-10 amino acid substitutions, 1-5 amino acid substitutions, or 1-3 amino acid substitutions. In some embodiments, the antibody comprises a VL comprising the amino acid sequence of SEQ ID No: 172 modified by 1-10 amino acid substitutions, 1-5 amino acid substitutions, or 1-3 amino acid substitutions. In some embodiments, the antibody comprises both a VH comprising the amino acid sequence of SEQ ID No: 162 modified by 1-10 amino acid substitutions, 1-5 amino acid substitutions, or 1-3 amino acid substitutions and a VL comprising the amino acid sequence of SEQ ID No: 172 modified by 1-10 amino acid substitutions, 1-5 amino acid substitutions, or 1-3 amino acid substitutions.

In some embodiments, the antibody comprises a VH comprising the amino acid sequence of SEQ ID No: 162. In some embodiments, the antibody comprises a VH comprising the amino acid sequence of SEQ ID No: 162; and a VL that is at least 90%, at least 95%, at least 97%, or at least 99% identical to the amino acid sequence of SEQ ID NO: 170. In some embodiments, the antibody comprises a VL comprising the amino acid sequence of SEQ ID No: 170. In some embodiments, the antibody comprises a VH that is at least 90%, at least 95%, at least 97%, or at least 99% identical to the amino acid sequence of SEQ ID NO: 162; and a VL comprising the amino acid sequence of SEQ ID No: 170. In some embodiments, the antibody comprises a VH comprising the amino acid sequence of SEQ ID No: 162 and a VL comprising the amino acid sequence of SEQ ID No: 170.

In some embodiments, the antibody comprises a VH comprising the amino acid sequence of SEQ ID No: 162. In some embodiments, the antibody comprises a VH comprising the amino acid sequence of SEQ ID No: 162; and a VL that is at least 90%, at least 95%, at least 97%, or at least 99% identical to the amino acid sequence of SEQ ID NO: 172. In some embodiments, the antibody comprises a VL comprising the amino acid sequence of SEQ ID No: 172. In some embodiments, the antibody comprises a VH that is at least 90%, at least 95%, at least 97%, or at least 99% identical to the amino acid sequence of SEQ ID NO: 162; and a VL comprising the amino acid sequence of SEQ ID No: 172. In some embodiments, the antibody comprises a VH comprising the amino acid sequence of SEQ ID No: 162 and a VL comprising the amino acid sequence of SEQ ID No: 172.

In some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of positions 26-35 of SEQ ID NO: 164, an HCDR2 comprising the amino acid sequence of positions 50-66 of SEQ ID NO: 164, and an HCDR3 comprising the amino acid sequence of positions 99-108 of SEQ ID NO: 164; and a light chain variable region (VL) comprising a light chain complementarity determining region 1 (LCDR1) comprising the amino acid sequence residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170. In some embodiments, the antibody comprises a VH that is at least 90%, at least 95%, at least 97%, or at least 99% identical to the amino acid sequence of SEQ ID NO: 164. In some embodiments, the antibody comprises a VL that is at least 90%, at least 95%, at least 97%, or at least 99% identical to the amino acid sequence of SEQ ID NO: 170. In some embodiments, the antibody comprises both a VH that is at least 90%, at least 95%, at least 97%, or at least 99% identical to the amino acid sequence of SEQ ID NO: 164; and a VL that is at least 90%, at least 95%, at least 97%, or at least 99% identical to the amino acid sequence of SEQ ID NO: 170. In some embodiments, the antibody comprises a VH comprising the amino acid sequence of SEQ ID No: 164 modified by 1-10 amino acid substitutions, 1-5 amino acid substitutions, or 1-3 amino acid substitutions. In some embodiments, the antibody comprises a VL comprising the amino acid sequence of SEQ ID No: 170 modified by 1-10 amino acid substitutions, 1-5 amino acid substitutions, or 1-3 amino acid substitutions. In some embodiments, the antibody comprises both a VH comprising the amino acid sequence of SEQ ID No: 164 modified by 1-10 amino acid substitutions, 1-5 amino acid substitutions, or 1-3 amino acid substitutions and a VL comprising the amino acid sequence of SEQ ID No: 170 modified by 1-10 amino acid substitutions, 1-5 amino acid substitutions, or 1-3 amino acid substitutions. In some embodiments, the antibody comprises a VL that is at least 90%, at least In some embodiments, the antibody comprises both a VH that is at least 90%, at least 95%, at least 97%, or at least 99% identical to the amino acid sequence of SEQ ID NO: 164; and a VL that is at least 90%, at least 95%, at least 97%, or at least 99% identical to the amino acid sequence of SEQ ID NO: 172. In some embodiments, the antibody comprises a VH comprising the amino acid sequence of SEQ ID No: 164 modified by 1-10 amino acid substitutions, 1-5 amino acid substitutions, or 1-3 amino acid substitutions. In some embodiments, the antibody comprises a VL comprising the amino acid sequence of SEQ ID No: 172 modified by 1-10 amino acid substitutions, 1-5 amino acid substitutions, or 1-3 amino acid substitutions. In some embodiments, the antibody comprises both a VH comprising the amino acid sequence of SEQ ID No: 164 modified by 1-10 amino acid substitutions, 1-5 amino acid substitutions, or 1-3 amino acid substitutions and a VL comprising the amino acid sequence of SEQ ID No: 172 modified by 1-10 amino acid substitutions, 1-5 amino acid substitutions, or 1-3 amino acid substitutions.

In some embodiments, the antibody comprises a VH comprising the amino acid sequence of SEQ ID No: 164. In some embodiments, the antibody comprises a VH comprising the amino acid sequence of SEQ ID No: 164; and a VL that is at least 90%, at least 95%, at least 97%, or at least 99% identical to the amino acid sequence of SEQ ID NO: 170. In some embodiments, the antibody comprises a VL comprising the amino acid sequence of SEQ ID No: 170. In some embodiments, the antibody comprises a VH that is at least 90%, at least 95%, at least 97%, or at least 99% identical to the amino acid sequence of SEQ ID NO: 164; and a VL comprising the amino acid sequence of SEQ ID No: 170. In some embodiments, the antibody comprises a VH comprising the amino acid sequence of SEQ ID No: 164 and a VL comprising the amino acid sequence of SEQ ID No: 170.

In some embodiments, the antibody comprises a VH comprising the amino acid sequence of SEQ ID No: 164. In some embodiments, the antibody comprises a VH comprising the amino acid sequence of SEQ ID No: 164; and a VL that is at least 90%, at least 95%, at least 97%, or at least 99% identical to the amino acid sequence of SEQ ID NO: 172. In some embodiments, the antibody comprises a VL comprising the amino acid sequence of SEQ ID No: 172. In some embodiments, the antibody comprises a VH that is at least 90%, at least 95%, at least 97%, or at least 99% identical to the amino acid sequence of SEQ ID NO: 164; and a VL comprising the amino acid sequence of SEQ ID No: 172. In some embodiments, the antibody comprises a VH comprising the amino acid sequence of SEQ ID No: 164 and a VL comprising the amino acid sequence of SEQ ID No: 172.

In some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of positions 26-35 of SEQ ID NO: 166, an HCDR2 comprising the amino acid sequence of positions 50-66 of SEQ ID NO: 166, and an HCDR3 comprising the amino acid sequence of positions 99-108 of SEQ ID NO: 166; and a light chain variable region (VL) comprising a light chain complementarity determining region 1 (LCDR1) comprising the amino acid sequence residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170. In some embodiments, the antibody comprises a VH that is at least 90%, at least 95%, at least 97%, or at least 99% identical to the amino acid sequence of SEQ ID NO: 166. In some embodiments, the antibody comprises a VL that is at least 90%, at least 95%, at least 97%, or at least 99% identical to the amino acid sequence of SEQ ID NO: 170. In some embodiments, the antibody comprises both a VH that is at least 90%, at least 95%, at least 97%, or at least 99% identical to the amino acid sequence of SEQ ID NO: 166; and a VL that is at least 90%, at least 95%, at least 97%, or at least 99% identical to the amino acid sequence of SEQ ID NO: 170. In some embodiments, the antibody comprises a VH comprising the amino acid sequence of SEQ ID No: 166 modified by 1-10 amino acid substitutions, 1-5 amino acid substitutions, or 1-3 amino acid substitutions. In some embodiments, the antibody comprises a VL comprising the amino acid sequence of SEQ ID No: 170 modified by 1-10 amino acid substitutions, 1-5 amino acid substitutions, or 1-3 amino acid substitutions. In some embodiments, the antibody comprises both a VH comprising the amino acid sequence of SEQ ID No: 166 modified by 1-10 amino acid substitutions, 1-5 amino acid substitutions, or 1-3 amino acid substitutions and a VL comprising the amino acid sequence of SEQ ID No: 170 modified by 1-10 amino acid substitutions, 1-5 amino acid substitutions, or 1-3 amino acid substitutions. In some embodiments, the antibody comprises a VL that is at least 90%, at least 95%, at least 97%, or at least 99% identical to the amino acid sequence of SEQ ID NO: 172. In some embodiments, the antibody comprises both a VH that is at least 90%, at least 95%, at least 97%, or at least 99% identical to the amino acid sequence of SEQ ID NO: 166; and a VL that is at least 90%, at least 95%, at least 97%, or at least 99% identical to the amino acid sequence of SEQ ID NO: 172. In some embodiments, the antibody comprises a VH comprising the amino acid sequence of SEQ ID No: 166 modified by 1-10 amino acid substitutions, 1-5 amino acid substitutions, or 1-3 amino acid substitutions. In some embodiments, the antibody comprises a VL comprising the amino acid sequence of SEQ ID No: 172 modified by 1-10 amino acid substitutions, 1-5 amino acid substitutions, or 1-3 amino acid substitutions. In some embodiments, the antibody comprises both a VH comprising the amino acid sequence of SEQ ID No: 166 modified by 1-10 amino acid substitutions, 1-5 amino acid substitutions, or 1-3 amino acid substitutions and a VL comprising the amino acid sequence of SEQ ID No: 172 modified by 1-10 amino acid substitutions, 1-5 amino acid substitutions, or 1-3 amino acid substitutions.

In some embodiments, the antibody comprises a VH comprising the amino acid sequence of SEQ ID No: 166. In some embodiments, the antibody comprises a VH comprising the amino acid sequence of SEQ ID No: 166; and a VL that is at least 90%, at least 95%, at least 97%, or at least 99% identical to the amino acid sequence of SEQ ID NO: 170. In some embodiments, the antibody comprises a VL comprising the amino acid sequence of SEQ ID No: 170. In some embodiments, the antibody comprises a VH that is at least 90%, at least 95%, at least 97%, or at least 99% identical to the amino acid sequence of SEQ ID NO: 166; and a VL comprising the amino acid sequence of SEQ ID No: 170. In some embodiments, the antibody comprises a VH comprising the amino acid sequence of SEQ ID No: 166 and a VL comprising the amino acid sequence of SEQ ID No: 170.

In some embodiments, the antibody comprises a VH comprising the amino acid sequence of SEQ ID No: 166. In some embodiments, the antibody comprises a VH comprising the amino acid sequence of SEQ ID No: 166; and a VL that is at least 90%, at least 95%, at least 97%, or at least 99% identical to the amino acid sequence of SEQ ID NO: 172. In some embodiments, the antibody comprises a VL comprising the amino acid sequence of SEQ ID No: 172. In some embodiments, the antibody comprises a VH that is at least 90%, at least 95%, at least 97%, or at least 99% identical to the amino acid sequence of SEQ ID NO: 166; and a VL comprising the amino acid sequence of SEQ ID No: 172. In some embodiments, the antibody comprises a VH comprising the amino acid sequence of SEQ ID No: 166 and a VL comprising the amino acid sequence of SEQ ID No: 172.

In some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of positions 26-35 of SEQ ID NO: 168, an HCDR2 comprising the amino acid sequence of positions 50-66 of SEQ ID NO: 168, and an HCDR3 comprising the amino acid sequence of positions 99-108 of SEQ ID NO: 168; and a light chain variable region (VL) comprising a light chain complementarity determining region 1 (LCDR1) comprising the amino acid sequence residues 24-38 of SEQ ID NO: 170, an LCDR2 comprising the amino acid sequence of residues 54-60 of SEQ ID NO: 170, and an LCDR3 comprising the amino acid sequence of residues 93-101 of SEQ ID NO: 170. In some embodiments, the antibody comprises a VH that is at least 90%, at least 95%, at least 97%, or at least 99% identical to the amino acid sequence of SEQ ID NO: 168. In some embodiments, the antibody comprises a VL that is at least 90%, at least 95%, at least 97%, or at least 99% identical to the amino acid sequence of SEQ ID NO: 170. In some embodiments, the antibody comprises both a VH that is at least 90%, at least 95%, at least 97%, or at least 99% identical to the amino acid sequence of SEQ ID NO: 168; and a VL that is at least 90%, at least 95%, at least 97%, or at least 99% identical to the amino acid sequence of SEQ ID NO: 170. In some embodiments, the antibody comprises a VH comprising the amino acid sequence of SEQ ID No: 168 modified by 1-10 amino acid substitutions, 1-5 amino acid substitutions, or 1-3 amino acid substitutions. In some embodiments, the antibody comprises a VL comprising the amino acid sequence of SEQ ID No: 170 modified by 1-10 amino acid substitutions, 1-5 amino acid substitutions, or 1-3 amino acid substitutions. In some embodiments, the antibody comprises both a VH comprising the amino acid sequence of SEQ ID No: 168 modified by 1-10 amino acid substitutions, 1-5 amino acid substitutions, or 1-3 amino acid substitutions and a VL comprising the amino acid sequence of SEQ ID No: 170 modified by 1-10 amino acid substitutions, 1-5 amino acid substitutions, or 1-3 amino acid substitutions. In some embodiments, the antibody comprises a VL that is at least 90%, at least 95%, at least 97%, or at least 99% identical to the amino acid sequence of SEQ ID NO: 172. In some embodiments, the antibody comprises both a VH that is at least 90%, at least 95%, at least 97%, or at least 99% identical to the amino acid sequence of SEQ ID NO: 168; and a VL that is at least 90%, at least 95%, at least 97%, or at least 99% identical to the amino acid sequence of SEQ ID NO: 172. In some embodiments, the antibody comprises a VH comprising the amino acid sequence of SEQ ID No: 168 modified by 1-10 amino acid substitutions, 1-5 amino acid substitutions, or 1-3 amino acid substitutions. In some embodiments, the antibody comprises a VL comprising the amino acid sequence of SEQ ID No: 172 modified by 1-10 amino acid substitutions, 1-5 amino acid substitutions, or 1-3 amino acid substitutions. In some embodiments, the antibody comprises both a VH comprising the amino acid sequence of SEQ ID No: 168 modified by 1-10 amino acid substitutions, 1-5 amino acid substitutions, or 1-3 amino acid substitutions and a VL comprising the amino acid sequence of SEQ ID No: 172 modified by 1-10 amino acid substitutions, 1-5 amino acid substitutions, or 1-3 amino acid substitutions.

In some embodiments, the antibody comprises a VH comprising the amino acid sequence of SEQ ID No: 168. In some embodiments, the antibody comprises a VH comprising the amino acid sequence of SEQ ID No: 168; and a VL that is at least 90%, at least 95%, at least 97%, or at least 99% identical to the amino acid sequence of SEQ ID NO: 170. In some embodiments, the antibody comprises a VL comprising the amino acid sequence of SEQ ID No: 170. In some embodiments, the antibody comprises a VH that is at least 90%, at least 95%, at least 97%, or at least 99% identical to the amino acid sequence of SEQ ID NO: 168; and a VL comprising the amino acid sequence of SEQ ID No: 170. In some embodiments, the antibody comprises a VH comprising the amino acid sequence of SEQ ID No: 168 and a VL comprising the amino acid sequence of SEQ ID No: 170.

In some embodiments, the antibody comprises a VH comprising the amino acid sequence of SEQ ID No: 168. In some embodiments, the antibody comprises a VH comprising the amino acid sequence of SEQ ID No: 168; and a VL that is at least 90%, at least 95%, at least 97%, or at least 99% identical to the amino acid sequence of SEQ ID NO: 172. In some embodiments, the antibody comprises a VL comprising the amino acid sequence of SEQ ID No: 172. In some embodiments, the antibody comprises a VH that is at least 90%, at least 95%, at least 97%, or at least 99% identical to the amino acid sequence of SEQ ID NO: 168; and a VL comprising the amino acid sequence of SEQ ID No: 172. In some embodiments, the antibody comprises a VH comprising the amino acid sequence of SEQ ID No: 168 and a VL comprising the amino acid sequence of SEQ ID No: 172.

In some cases, the antibody is an IgG antibody, such as a human IgG1, IgG2, IgG3, or IgG4 antibody or a murine IgG1 or IgG2 antibody. In some cases, the antibody comprises a wild-type, human IgG1, IgG2, or IgG4 heavy chain constant region. In some embodiments, the antibody comprises a full length heavy chain and/or a full length light chain. In other cases, the antibody lacks a C-terminal lysine at the end of the heavy chain constant region. In yet other cases, the antibody lacks a C-terminal glycine-lysine at the end of the heavy chain constant region. In some cases, the antibody is an antibody fragment, such as an Fv, single-chain Fv (scFv), Fab, Fab′, or (Fab′)₂.

In some embodiments, the antibody is a bispecific or multispecific antibody, or which is conjugated covalently or noncovalently to at least one other molecule. In some embodiments, the antibody is conjugated covalently or noncovalently to at least one other molecule, wherein the at least one other molecule comprises a detection label and/or a drug.

In some embodiments, the antibody comprises a human IgG1 heavy chain constant region comprising an amino acid sequence at least 90%, at least 95%, at least 97%, or at least 99% identical to the amino acid sequence of any one of SEQ ID Nos: 174, 176, 178, 180, 182, 184, 186, 188, 190, or 192. In some embodiments, the antibody comprises a light chain constant region comprising an amino acid sequence at least 90%, at least 95%, at least 97%, or at least 99% identical to the amino acid sequence of SEQ ID NO: 194. In some embodiments, the antibody comprises both a human IgG1 heavy chain constant region comprising an amino acid sequence at least 90%, at least 95%, at least 97%, or at least 99% identical to the amino acid sequence of any one of SEQ ID Nos: 174, 176, 178, 180, 182, 184, 186, 188, 190, or 192; and a light chain constant region comprising an amino acid sequence at least 90%, at least 95%, at least 97%, or at least 99% identical to the amino acid sequence of SEQ ID NO: 194. In some cases, the antibody comprises a heavy chain constant region comprising an amino acid sequence modified by 1-10 amino acid substitutions, 1-5 amino acid substitutions, or 1-3 amino acid substitutions compared to the amino acid sequence of any one of SEQ ID Nos: 174, 176, 178, 180, 182, 184, 186, 188, 190, or 192. In some cases, the antibody comprises a light chain constant region comprising an amino acid sequence modified by 1-10 amino acid substitutions, 1-5 amino acid substitutions, or 1-3 amino acid substitutions compared to the amino acid sequence of SEQ ID NO: 194. In some cases, the antibody comprises both a heavy chain constant region comprising an amino acid sequence modified by 1-10 amino acid substitutions, 1-5 amino acid substitutions, or 1-3 amino acid substitutions compared to the amino acid sequence of any one of SEQ ID Nos: 174, 176, 178, 180, 182, 184, 186, 188, 190, or 192; and a light chain constant region comprising an amino acid sequence modified by 1-10 amino acid substitutions, 1-5 amino acid substitutions, or 1-3 amino acid substitutions compared to the amino acid sequence of SEQ ID NO: 194. In some embodiments, the antibody comprises a human IgG1 heavy chain constant region comprising amino acid sequence of any one of SEQ ID Nos: 174, 176, 178, 180, 182, 184, 186, 188, 190, or 192. In some embodiments, the antibody comprises a light chain constant region comprising the amino acid sequence of SEQ ID NO: 194. In some embodiments, the antibody comprises both a human IgG1 heavy chain constant region comprising the amino acid sequence of any one of SEQ ID Nos: 174, 176, 178, 180, 182, 184, 186, 188, 190, or 192; and a light chain constant region comprising the amino acid sequence of SEQ ID NO: 194.

Murine Antibodies

In some embodiments, the disclosure relates to an isolated antibody that specifically binds to murine protein arginine deiminase 4 (PAD4), wherein the antibody comprises a VH comprising a HCDR1 comprising the amino acid sequence of positions 31-35 of SEQ ID NO: 208, an HCDR2 comprising the amino acid sequence of positions 50-66 of SEQ ID NO: 208, and an HCDR3 comprising the amino acid sequence of positions 99-107 of SEQ ID NO: 208; and wherein the antibody comprises a light chain variable region (VL) comprising a light chain complementarity determining region 1 (LCDR1) comprising the amino acid sequence of residues 24-38 of SEQ ID NO: 210, an LCDR2 comprising the amino acid sequence of residues 54-60 of SEQ ID NO: 210, and an LCDR3 comprising the amino acid sequence of residues 93-101 of SEQ ID NO: 210.

In some embodiments, the antibody comprises a VH that is at least 90%, at least 95%, at least 97%, or at least 99% identical to the amino acid sequence of SEQ ID No: 208. In some embodiments. In some embodiments, the antibody comprises a VL that is at least 90%, at least 95%, at least 97%, or at least 99% identical to the amino acid sequence of SEQ ID No: 210. In some embodiments, the antibody comprises both a VH that is at least 90%, at least 95%, at least 97%, or at least 99% identical to the amino acid sequence of SEQ ID No: 208; and a VL that is at least 90%, at least 95%, at least 97%, or at least 99% identical to the amino acid sequence of SEQ ID No: 210. In some embodiments, the antibody comprises a VH comprising the amino acid of SEQ ID No: 208 modified by 1-10 amino acid substitutions, 1-5 amino acid substitutions, or 1-3 amino acid substitutions. In some embodiments, the antibody comprises a VL comprising the amino acid sequence of any one of SEQ ID Nos: 210 modified by 1-10 amino acid substitutions, 1-5 amino acid substitutions, or 1-3 amino acid substitutions. In some embodiments, the antibody comprises both a VH comprising the amino acid of SEQ ID No: 208 modified by 1-10 amino acid substitutions, 1-5 amino acid substitutions, or 1-3 amino acid substitutions; and a VL comprising the amino acid sequence of any one of SEQ ID Nos: 210 modified by 1-10 amino acid substitutions, 1-5 amino acid substitutions, or 1-3 amino acid substitutions. In some such cases, the antibody further comprises an HCDR1 comprising the amino acid sequence of positions 31-35 of SEQ ID NO: 208, an HCDR2 comprising the amino acid sequence of positions 50-66 of SEQ ID NO: 208, and an HCDR3 comprising the amino acid sequence of positions 99-107 of SEQ ID NO: 208, an LCDR1 comprising the amino acid sequence of residues 24-38 of SEQ ID NO: 210, an LCDR2 comprising the amino acid sequence of residues 54-60 of SEQ ID NO: 210, and an LCDR3 comprising the amino acid sequence of residues 93-101 of SEQ ID NO: 210.

In some embodiments, the antibody comprises a VH comprising the amino acid sequence of SEQ ID NO: 208. In some embodiments, the antibody comprises a VL comprising the amino acid sequence of SEQ ID NO: 210. In some embodiments, the antibody comprises a VH comprising the amino acid sequence of SEQ ID NO: 208 and a VL comprising the amino acid sequence of SEQ ID NO: 210.

In some embodiments herein, the antibody is an IgA, IgG, or IgM antibody. In some cases, the antibody is a murine IgG1 or IgG2 antibody. In some embodiments, the antibody comprises a full length heavy chain and/or a full length light chain. In other cases, the antibody lacks a C-terminal lysine at the end of the heavy chain constant region. In yet other cases, the antibody lacks a C-terminal glycine-lysine at the end of the heavy chain constant region. In some cases, the antibody is an antibody fragment, such as an Fv, single-chain Fv (scFv), Fab, Fab′, or (Fab′)₂.

In some embodiments, the antibody is a bispecific or multispecific antibody, or which is conjugated covalently or noncovalently to at least one other molecule. In some embodiments, the antibody is conjugated covalently or noncovalently to at least one other molecule, wherein the at least one other molecule comprises a detection label and/or a drug.

In some embodiments, the antibody comprises a HC comprising the amino acid sequence of SEQ ID No: 212. In some embodiments, the antibody comprises a LC comprising the amino acid sequence of SEQ ID No: 214. In some embodiments, the antibody comprises both a HC comprising the amino acid sequence of SEQ ID No: 212, and a LC comprising the amino acid sequence of SEQ ID No: 214.

### III. Exemplary Antibody Variants, Fragments, and Constant Regions

In many embodiments, an antibody specifically binding to PAD4 may further incorporate any of the features, singly or in combination, as described in the sections that follow.

A. Antibody Fragments

In certain embodiments, an antibody provided herein is an antibody fragment. Antibody fragments include, but are not limited to, Fab, Fab′, Fab′-SH, F(ab′)2, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,′87,458. For discussion of Fab and F(a″)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

B. Bispecific or Multispecific Antibodies

In certain embodiments, an antibody provided herein is a multispecific antibody, for example, a bispecific antibody. Multispecific antibodies are monoclonal antibodies that have binding specificities for at least two different sites. In certain embodiments, one of the binding specificities is TREM2 and the other is for any other antigen. In certain embodiments, bispecific antibodies may bind to two different epitopes of TREM2. Bispecific antibodies may also be used to localize drugs such as cytotoxic agents or to localize detection labels to cells that express TREM2. some embodiments, the multispecific antibody (e.g., bispecific antibody) comprises a first variable domain comprising the CDRs or variable regions as described herein. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 18(5):1547-1553 (1992)); using“diabod” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576).

C. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

In some embodiments, the humanized antibodies may comprise a human IgG1, IgG2, IgG3, or IgG4 heavy chain constant region.

D. Glycosylation and Pegylation Variants

In certain embodiments, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for antigen. Such an approach is described in further detail in U.S. Pat. Nos. 5,714,350 and 6,350,861 by Co et al.

Glycosylation of the constant region on N297 can be prevented by mutating the N297 residue to another residue, e.g., N297A, and/or by mutating an adjacent amino acid, e.g., 298 to thereby reduce glycosylation on N297.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies described herein to thereby produce an antibody with altered glycosylation. For example, EP 1,176,195 by Hanai et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Led 3 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases {e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17: 176-180).

Another modification of the antibodies described herein is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. In some embodiments, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (CI-CIO) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies described herein. See for example, EP 0 154 316 by Nishimura et al. and EP 0 401 384 by Ishikawa et al.

E. Constant Regions

In some embodiments, an antibody described herein comprises one or more human constant regions. In some embodiments, the human heavy chain constant region is of an isotype selected from IgA, IgG, and IgD. In some embodiments, the human light chain constant region is of an isotype selected from κ and λ. In some embodiments, an antibody described herein comprises a human IgG constant region, such as an IgG1, IgG2, IgG3, or IgG4. In some embodiments, an antibody described herein comprises a human IgG4 heavy chain constant region. In some such embodiments, an antibody described herein comprises an S241P mutation in the human IgG4 constant region. In some embodiments, an antibody described herein comprises a human IgG4 constant region and a human κ light chain.

The choice of heavy chain constant region can determine whether or not an antibody will have effector function in vivo. Such effector function, in some embodiments, includes antibody-dependent cell-mediated cytotoxicity (ADCC) and/or complement-dependent cytotoxicity (CDC), and can result in killing of the cell to which the antibody is bound. In some methods of treatment, including methods of treating some cancers, cell killing may be desirable, for example, when the antibody binds to a cell that supports the maintenance or growth of the tumor. Exemplary cells that may support the maintenance or growth of a tumor include, but are not limited to, tumor cells themselves, cells that aid in the recruitment of vasculature to the tumor, and cells that provide ligands, growth factors, or counter-receptors that support or promote tumor growth or tumor survival. In some embodiments, when effector function is desirable, an antibody comprising a human IgG1 heavy chain or a human IgG3 heavy chain is selected.

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibodies with certain improved properties. For example, in some embodiments an antibody may be afucosylated, for example, by mutating residues such as Asn297 that are normally glycosylated with fucose-containing glycosylations, or through other means. In some embodiments, antibodies herein may comprise an afucosylated human IgG1 constant region.

Antibodies are further provided with bisected oligosaccharides, e.g., in which a biantennary oligosaccharide attached to the Fc region of the antibody is bisected by GlcNAc. Such antibodies may have reduced fucosylation and/or improved ADCC function. Examples of such antibodies are described, e.g., in WO 2003/011878 (Jean-Mairet et al.); U.S. Pat. No. 6,602,684 (Umana et al.); and US 2005/0123546 (Umana et al.). Antibodies with at least one galactose residue in the oligosaccharide attached to the Fc region are also provided. Such antibodies may have improved CDC function. Such antibodies are described, e.g., in WO 1997/30087 (Patel et al.); WO 1998/58964 (Raju, S.); and WO 1999/22764 (Raju, S.).

Antibodies are also provided with amino-terminal leader extensions. For example, one or more amino acid residues of the amino-terminal leader sequence are present at the amino-terminus of any one or more heavy or light chains of an antibody. An exemplary amino-terminal leader extension comprises or consists of three amino acid residues, VHS, present on one or both light chains of an antibody.

The in vivo or serum half-life of human FcRn high affinity binding polypeptides can be assayed, e.g., in transgenic mice, in humans, or in non-human primates to which the polypeptides with a variant Fc region are administered. See also, e.g., Petkova et al. International Immunology 18(12):1759-1769 (2006).

In some embodiments of the invention, an afucosylated antibody mediates ADCC in the presence of human effector cells more effectively than a parent antibody that comprises fucose, Generally, ADCC activity may be determined using the in vitro ADCC assay as herein disclosed, but other assays or methods for determining ADCC activity, e.g. in an animal model etc., are contemplated.

In certain embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320, 322, 330, and/or 331 (EU numbering) can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In some examples, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In some examples, one or more amino acid residues within amino acid positions 231 and 239 are altered to thereby alter the ability of the antibody to fix complement. This approach is described further in PCT Publication WO 94/29351 by Bodmer et al. In some examples, the Fc region can be modified to decrease antibody dependent cellular cytotoxicity (ADCC) and/or to decrease the affinity for an Fcγ receptor by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439 (EU numbering). Exemplary substitutions include 236A, 239D, 239E, 268D, 267E, 268E, 268F, 324T, 332D, and 332E. Exemplary variants include 239D/332E, 236A/332E, 236A/239D/332E, 268F/324T, 267E/268F, 267E/324T, and 267E/268F7324T (EU numbering). Other Fc modifications that can be made to Fcs are those for reducing or ablating binding to FcγR and/or complement proteins, thereby reducing or ablating Fc-mediated effector functions such as ADCC, ADCP, and CDC. Exemplary modifications include but are not limited substitutions, insertions, and deletions at positions 234, 235, 236, 237, 267, 269, 325, 328, 330, and/or 331 (e.g., 330 and 331), wherein numbering is according to the EU index. Exemplary substitutions include but are not limited to 234A, 235E, 236R, 237A, 267R, 269R, 325L, 328R, 330S, and 331S (e.g., 330S, and 331S), wherein numbering is according to the EU index. An Fc variant can comprise 236R/328R. Other modifications for reducing FcγR and complement interactions include substitutions 297A, 234A, 235A, 237A, 318A, 228P, 236E, 268Q, 309L, 330S, 331 S, 220S, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691. For example, the human IgG1.3 Fc constant region contains L234A, L235E, and G237A substitutions. The IgG1fa.P238K (or IgG1.P238K) contains a P238K substitution. The IgG1.1f comprises L234A, L235E, G237A, A330S, and P331S substitutions. (All numbering under the EU index.)

Fc variants that enhance affinity for an inhibitory receptor FcγRIIb can also be used. Such variants can provide an Fc fusion protein with immunomodulatory activities related to FcγRIIb cells, including for example B cells and monocytes. In one embodiment, the Fc variants provide selectively enhanced affinity to FcγRIIb relative to one or more activating receptors. Modifications for altering binding to FcγRIIb include one or more modifications at a position selected from the group consisting of 234, 235, 236, 237, 239, 266, 267, 268, 325, 326, 327, 328, 330, 331, and 332, according to the EU index. Exemplary substitutions for enhancing FcγRIIb affinity include but are not limited to 234A, 234D, 234E, 234F, 234W, 235D, 235E, 235F, 235R, 235Y, 236D, 236N, 237A, 237D, 237N, 239D, 239E, 266M, 267D, 267E, 268D, 268E, 327D, 327E, 328F, 328W, 328Y, 330S, 331S, and 332E. Exemplary substitutions include 235Y, 236D, 239D, 266M, 267E, 268D, 268E, 328F, 328W, and 328Y. Other Fc variants for enhancing binding to FcγRIIb include 235Y/267E, 236D/267E, 239D/268D, 239D/267E, 267E/268D, 267E/268E, and 267E/328F. (All numbering under the EU index.)

Other modifications for enhancing FcγR and complement interactions include but are not limited to substitutions 298A, 333A, 334A, 326A, 2471, 339D, 339Q, 280H, 290S, 298D, 298V, 243L, 292P, 300L, 396L, 3051, and 396L. These and other modifications are reviewed in Strohl, 2009, Current Opinion in Biotechnology 20:685-691. Fc modifications that increase binding to an Fcγ receptor include amino acid modifications at any one or more of amino acid positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 324, 327, 329, 330, 335, 337, 338, 340, 360, 373, 376, 379, 382, 388, 389, 398, 414, 416, 419, 430, 434, 435, 437, 438 or 439 of the Fc region, wherein the numbering of the residues in the Fc region is that of the EU index as in Patent Publication No. WO 00/42072.

Optionally, the Fc region can comprise a non-naturally occurring amino acid residue at additional and/or alternative positions known to one skilled in the art (see, e.g., U.S. Pat. Nos. 5,624,821; 6,277,375; 6,737,056; 6,194,551; 7,317,091; 8,101,720; PCX Patent Publications WO 00/42072; WO 01/58957; WO 02/06919; WO 04/016750; WO 04/029207; WO 04/035752; WO 04/074455; WO 04/099249; WO 04/063351; WO 05/070963; WO 05/040217, WO 05/092925 and WO 06/0201 14).

The affinities and binding properties of an Fc region for its ligand can be determined by a variety of in vitro assay methods (biochemical or immunological based assays) known in the art including but not limited to, equilibrium methods (e.g., enzyme-linked immunoabsorbent assay (ELISA), or radioimmunoassay (RIA)), or kinetics (e.g., BIACORE analysis), and other methods such as indirect binding assays, competitive inhibition assays, fluorescence resonance energy transfer (FRET), gel electrophoresis and chromatography (e.g., gel filtration). These and other methods can utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental immunology, 4th Ed., Lippincott-Raven, Philadelphia (1999), which focuses on antibody-immunogen interactions.

In certain embodiments, the antibody is modified to increase its biological half-life. Various approaches are possible. For example, this can be done by increasing the binding affinity of the Fc region for FcRn, For example, one or more of more of following residues can be mutated: 252, 254, 256, 433, 435, 436, as described in U.S. Pat. No. 6,277,375. Specific exemplary substitutions include one or more of the following: T252L, T254S, and/or T256F. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Other exemplary variants that increase binding to FcRn and/or improve pharmacokinetic properties include substitutions at positions 259, 308, 428, and 434, including for example 2591, 308F, 428L, 428M, 434S, 4341 1. 434F, 434Y, and 434X1. Other variants that increase Fc binding to FcRn include: 250E, 250Q, 428 L, 428F, 250Q/428L (Hinton et al. 2004, J. Biol. Chem. 279(8): 6213-6216, Hinton et al. 2006 Journal of Immunology 176:346-356), 256A, 272A, 286A, 305A, 307A, 307Q, 311A, 312A, 376A, 378Q, 380A, 382A, 434A (Shields et al., Journal of Biological Chemistry, 2001, 276(9):6591-6604), 252F, 252T, 252Y, 252W, 254T, 256S, 256R, 256Q, 256E, 256D, 256T, 309P, 311S, 433R, 433S, 4331, 433P, 433Q, 434H, 434F, 434Y, 252Y/254T/256E, 433K/434F/436H, 308T/309P/311S (Dall'Acqua et al. Journal of Immunology, 2002, 169:5171-5180, Dal'Acqua et al., 2006, Journal of Biological Chemistry 281:23514-23524). Other modifications for modulating FcRn binding are described in Yeung et al., 2010, J Immunol, 182:7663-7671.

In certain embodiments, hybrid IgG isotypes with particular biological characteristics can be used. For example, an IgG1/IgG3 hybrid variant can be constructed by substituting IgG1 positions in the CH2 and/or CH3 region with the amino acids from IgG3 at positions where the two isotypes differ. Thus a hybrid variant IgG antibody can be constructed that comprises one or more substitutions, e.g., 274Q, 276K, 300F, 339T, 356E, 358M, 384S, 392N, 397M, 4221, 435R, and 436F. In some embodiments described herein, an IgG1/IgG2 hybrid variant can be constructed by substituting IgG2 positions in the CH2 and/or CH3 region with amino acids from IgG1 at positions where the two isotypes differ. Thus a hybrid variant IgG antibody can be constructed that comprises one or more substitutions, e.g., one or more of the following amino acid substitutions: 233E, 234L, 235L, +236G (referring to an insertion of a glycine at position 236), and 327A.

Moreover, the binding sites on human IgG1 for FcγRI, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A, which has been shown to exhibit enhanced FcγRIIIa binding and ADCC activity (Shields et al., 2001). Other IgG1 variants with strongly enhanced binding to FcγRIIIa have been identified, including variants with S239D/I332E and S239D/I332E/A330L mutations which showed the greatest increase in affinity for FcγRIIIa, a decrease in FcγRIIb binding, and strong cytotoxic activity in cynomolgus monkeys (Lazar et al., 2006). Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/I332E variant showed an enhanced capacity to deplete B cells in monkeys (Lazar et al., 2006). In addition, IgG1 mutants containing L235V, F243L, R292P, Y300L and P396L mutations which exhibited enhanced binding to FcγRIIIa and concomitantly enhanced ADCC activity in transgenic mice expressing human FcγRIIIa in models of B cell malignancies and breast cancer have been identified (Stavenhagen et al., 2007; Nordstrom et al., 2011). Other Fc mutants that can be used include: S298A/E333A/L334A, S239D/I332E, S239D/I332E/A330L, L235V/F243L/R292P/Y300L/P396L, and M428L/N434S.

In certain embodiments, an Fc is chosen that has reduced binding to FcγRs. An exemplary Fc, e.g., IgG1 Fc, with reduced FcγR binding comprises the following three amino acid substitutions: L234A, L235E and G237A.

In certain embodiments, an Fc is chosen that has reduced complement fixation. An exemplary Fc, e.g., IgG1 Fc, with reduced complement fixation has the following two amino acid substitutions: A330S and P331S.

In certain embodiments, an Fc is chosen that has essentially no effector function, i.e., it has reduced binding to FcγRs and reduced complement fixation. An exemplary Fc, e.g., IgG1 Fc, that is effectorless comprises the following five mutations: L234A, L235E, G237A, A330S and P331S.

When using an IgG4 constant domain, it can include the substitution S228P, which mimics the hinge sequence in IgG1 and thereby stabilizes IgG4 molecules. Fc modifications described in WO 2017/087678 or WO2016081746 may also be used.

In certain embodiments, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for antigen. Such an approach is described in further detail in U.S. Pat. Nos. 5,714,350 and 6,350,861 by Co et al.

Glycosylation of the constant region on N297 can be prevented by mutating the N297 residue to another residue, e.g., N297A, and/or by mutating an adjacent amino acid, e.g., 298 to thereby reduce glycosylation on N297.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies described herein to thereby produce an antibody with altered glycosylation. For example, EP 1,176,195 by Hanai et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Led 3 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases {e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17: 176-180).

Another modification of the antibodies described herein is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. In some embodiments, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (CI-CIO) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies described herein. See for example, EP 0 154 316 by Nishimura et al. and EP 0 401 384 by Ishikawa et al.

### IV. Exemplary Properties of Particular Anti-PAD4 Antibodies

In many embodiments, an antibody specifically binding to PAD4 may comprise any of the following properties, singly or in combination.

In some cases, binding of an antibody to a ligand such as PAD4 may be determined by surface plasmon resonance (SPR). In some embodiments, the antibody specifically binds to PAD4 in an SPR assay with a K_{D} less than 5 nM, less than 1 nM, less than 0.5 nM, less than 0.1 nM, from 0.01 nM to 5 nM, from 0.01 nM to 1 nM, from 0.05 nM to 1 nM, from 0.1 nM to 1 nM, from 0.1 nM to 0.5 nM, from 0.05 nM to 0.5 nM, from 0.05 nM to 0.1 nM, or from 0.5 nM to 1 nM. For example, such a K_{D} may be obtained by an SPR assay. In some embodiments, the antibody specifically binds to PAD4 in an SPR assay with a K_{D} less than 5 nM, less than 1 nM, less than 0.5 nM, less than 0.1 nM, from 0.01 nM to 5 nM, from 0.01 nM to 1 nM, from 0.05 nM to 1 nM, from 0.1 nM to 1 nM, from 0.1 nM to 0.5 nM, from 0.05 nM to 0.5 nM, from 0.05 nM to 0.1 nM, or from 0.5 nM to 1 nM in the presence of 1-2 mM calcium chloride (e.g., in the presence of 1 mM calcium chloride). In some embodiments, the antibody specifically binds to PAD4 by SPR with a K_{D} less than 5 nM, less than 1 nM, less than 0.5 nM, less than 0.1 nM, from 0.01 nM to 5 nM, from 0.01 nM to 1 nM, from 0.05 nM to 1 nM, from 0.1 nM to 1 nM, from 0.1 nM to 0.5 nM, from 0.05 nM to 0.5 nM, from 0.05 nM to 0.1 nM, or from 0.5 nM to 1 nM both in the presence of 1-2 mM calcium chloride (e.g., 1 mM calcium chloride) and in the absence of calcium ion (due to absence of added calcium salt as well as the presence of EDTA, such as 1 mM or 2 mM EDTA). Thus, in such cases, the antibody specifically binds to PAD4 in the presence and absence of calcium ion such as calcium chloride. See, for instance, Tables 2 and 3 herein for examples.

In some embodiments, the anti-PAD4 antibody does not bind to human protein arginine deiminase 2 (PAD2). Thus, in a binding assay such as by SPR, binding to PAD2 is too weak to be detected. In some cases, the antibody specifically binds to human PAD4, for example, as described just above, but does not bind to murine PAD4 (i.e., binding to murine PAD4 is not detected in an SPR or similar assay). In some cases, the antibody specifically binds to human PAD4, for example, as described just above, but does not bind to cynomolgus PAD4 (i.e., binding to cynomolgus PAD4 is not detected in an SPR assay), while in other cases, the antibody specifically binds to both human and cynomolgus PAD4 in an SPR assay. In some cases, the antibody binds specifically to both human and cynomolgus PAD4 but not to murine PAD4 by SPR. In other cases, the antibody binds specifically to human PAD4 but not to cynomolgus or murine PAD4 as determined by SPR.

In some embodiments, the antibody has an ECM score of less than 50, less than 30, less than 10, less than 5, 1-30, 1-20, 1-10, 1-5, 1-3, 1, 2, 3, 4, or 5 in an ECM assay. (See, for example Table 5.) As described in Example 5 below, ECM scores indicate the degree to which an antibody binds nonspecifically to the extracellular matrix (ECM). An assay may be conducted using pre-coated ECM plates, such as a commercially available 96-well plate. ECM scores can be determined after a 1 hour incubation of antibody with an ECM-coated plate, subsequent incubation with HRP conjugated detection antibody and reaction with TMB substrate, and then dividing the absorbance value measured at 450 nm by that of a control well with no antibody addition. (See Example 5 below.)

In some embodiments, an antibody herein has a hydrophobic interaction chromatography (HIC) retention time of 9-11 minutes on a TSKgel Butyl-NPR column (4.6 mm×3.5 cm, 2.5 μm particle size, Tosoh P/N 14947) on an Agilent 1260 Infinity II HPLC system using a linear gradient of mobile phase A (0.1 M sodium phosphate pH 7.0, 2 M ammonium sulfate) and mobile phase B (solution 0.1 M sodium phosphate pH 7.0) for 20 min at a flow rate of 1.0 ml/min at 25° C. column temperature. (See Example 6 below.) In some embodiments, the antibody has a melting temperature (Tm) between 60 and 70° C. at both pH 6.0 and pH 8.3, as measured by intrinsic fluorescence. In some embodiments, the antibody has an aggregation temperature (Tagg) between 64 and 75° C. at pH 6.0 and between 60 and 70° C. at pH 8.3, as measured by static light scattering. (See Example 7.)

In some embodiments, the antibody has a relatively low immunogenicity as measured using an Epivax® immunogenicity test. The commercial in silico immunogenicity risk assessment algorithm (Epivax®) ranks peptide MHC class II binding across 8 human HLA DRB1 allele super types to cover >90% of the variability present in the human population (De Groot and Martin, Clin Immunol, 2009, 131(2): p. 189-201.) (See Example 15.) For example, the in silico immunogenicity score for the drugs Campath® (alemtuzumab), Rituxan® (rituximab), Zenapax® (daclizumab), Humicade® (CDP-571), Mylotarg® (gemtuzumab), and Avastin® (bevacizumab) ranges from 0% to 45%, as shown in Table 10 below, with Campath® (alemtuzumab), Rituxan® (rituximab), Zenapax® (daclizumab), and Humicade® (CDP-571) each from 45% to 7%, respectively. In contrast, in some embodiments, antibodies herein have immunogenicity scores ranging from 0-5%, such as 0-2%, or 0-1% in the same assay. Certain antibodies herein have immunogenicity scores of 0%. (See Table 10.) Thus, in certain cases, the antibody is less immunogenic than one, two, or all three of Campath® (alemtuzumab), Rituxan® (rituximab), and Zenapax® (daclizumab), as measured in an in silico immunogenicity assay.

In some embodiments, the antibody inhibits the enzymatic activity of PAD4 in vitro. For example, in some cases, the antibody inhibits PAD4 conversion of arginine in peptide substrate TSTGGRQGSHH (SEQ ID NO: 216) to citrulline with an IC50 of 10-200 nM, 50-200 nM, 10-100 nM, 20-100 nM, or 50-100 nM in vitro. (See Example 4, Table 4.) For example, in some cases, the antibody inhibits PAD4 conversion of arginine in peptide substrate (SHQESTRGKSKGKAAAAA; SEQ ID NO: 232) to citrulline in vitro. For example, in some cases, it does so with an IC50 of 0.1-10 nM, such as 0.2-5 nM, optionally in a dose-dependent manner with a PAD4 concentration of 1-8 μg/mL. (See, for example, Example 16 below.)

In some embodiments, the antibody inhibits the enzymatic activity of PAD4 in vitro in the presence of an endogenous anti-PAD4 antibody (e.g., a polyclonal mixture of endogenous anti-PAD4 antibodies) from a human subject (e.g., a patient with a disease, e.g., a disease disclosed herein, e.g., an inflammatory or autoimmune disease, e.g. rheumatoid arthritis). The endogenous anti-PAD4 antibody can include or be, e.g., a PAD4 inhibiting antibody and/or a PAD4 activating antibody. In some embodiments, the antibody inhibits the enzymatic activity of PAD4 in vitro in the presence of an endogenous antibody that binds and activates human PAD4 (an activating antibody). In some embodiments, the activating antibody is an antibody derived from a patient with rheumatoid arthritis. In some embodiments, the activating antibody is an antibody that is cross reactive for human PAD3 and human PAD4.

In some cases, the antibody reduces the amount of extracellular citrullinated histone H3 in LPS-stimulated human blood monocytes compared to an isotype control antibody. In some cases, the antibody reduces secretion of GM-CSF in LPS-stimulated human blood monocytes compared to an isotype control antibody. In some cases, the antibody reduces gene expression of GM-CSF in LPS-stimulated human blood monocytes compared to an isotype control antibody. In some cases, the antibody has two of these three properties. In some cases, the antibody has all three of these properties. In some embodiments, the LPS-stimulated human blood monocytes are CD14+CD16− monocytes isolated from fresh human PBMCs. (See Example 17 herein.) In some embodiments, the antibody is capable of being internalized by LPS-stimulated human blood monocytes. For example, an antibody may be labeled with a dye to detect internalization by monocytes. In some cases, the antibody may be internalized by LPS-stimulated CD14+ human monocytes. (See Example 18.) Such results indicate that anti-PAD4 antibodies herein can act intracellularly in monocytes, and in some embodiments may block functions of PAD4, both extracellular and intracellular.

In some embodiments, the antibody inhibits PAD4 function in an inflamed lung. For example, this may be demonstrated by decreased citrullination of histone H3 or ITIH4 in broncheoalveolar lavage fluid (BALF) collected from the lung, as described in Example 20 or 25 herein. For example, BALF may be collected from mice with acute or chronic lung inflammation and assayed for the amount of citrullinated H3 or ITIH4 protein in the presence of the antibody and in the presence of a wild-type control. In some embodiments, the mice are human PAD4 knock-in mice. In some cases, the amount of citrullinated H3 is reduced by at least 15%, at least 30%, at least 40%, at least 50%, or at least 60%, and/or the amount of citrullinated ITIH4 is reduced by at least 50%, at least 70%, or at least 80% in the presence of the antibody in human PAD4 knock-in mice, compared to an isotope control (IC) antibody, for example, as calculated by the following formula: % inhibition=[(IC−naïve)−(PAD4 mAb−naïve)/(IC−naïve)]×100, in which the amount of citrullinated H3 or ITIH4 is measured for untreated (naïve) mice, mice receiving IC, and mice receiving an anti-PAD4 antibody.

In some embodiments, the antibody inhibits PAD4 function in an inflamed joint, for instance, as described in Examples 21, 22, and/or 26. This may be demonstrated, for example, by decreased citrullination of ITIH4 and/or PRG4 in joint tissue in a mouse model with acute or chronic joint inflammation, such as induced by LPS injection of a joint. In some cases, the mice may be human PAD4 knock-in mice. In some cases, the antibody reduces the amount of extracellular citrullinated PRG4 and/or citrullinated ITIH4 in mouse patella in an LPS-induced acute joint injury model with human PAD4 knock-in mice compared to an isotype control antibody. In some cases, the EC50 of reduction of citrullinated ITIH4 and/or citrullinated PRG4 is 2 nM or lower, 1 nM or lower, 0.1 nM or lower, 0.05-2 nM, or 0.1-1 nM.

In some embodiments, an antibody herein may reduce pristane-induced extracellular trap forming neutrophils (NETosis) and/or extracellular trap forming monocytes (METosis). (See Example 23.) METosis is the process by which extracellular traps composed of cellular DNA studded with histones and cellular proteins are released from monocytes or macrophages. This net-like material formed by either METosis or NETosis (neutrophil origin) is important in defense against microbes but is also primary drivers of autoimmune pathology and aseptic inflammation. For example, mice, such as human PAD4 knock-in mice, may be injected intraperitoneally with pristane, for example, following treatment with an antibody herein or with an isotype control antibody. In some cases, in such a pristane-induced mouse model, an antibody herein may reduce citrullination of H3 in neutrophils, monocytes, M1 macrophages, and/or M2 macrophages in peritoneal fluid compared to an isotype control antibody. In further cases, the antibody may also reduce the amount of soluble markers of neutrophils and monocytes/macrophages in the mice in peritoneal fluid, such as elastase, MPO, MIP-2 alpha, GRO alpha/KC, MCP1, MIP 1beta, IL6, and MIP3 alpha. (See Example 23.)

In some embodiments, in a collagen-induced arthritis mouse model, an antibody herein may inhibit PAD4-dependent responses. (See Example 24.) For example, in some embodiments, an antibody herein significantly reduces the arthritis clinical score of mice in the arthritis model compared to an isotype control, according to the following scale: (1) normal; (2) mild, with definite redness and swelling of the ankle or wrist, or with apparent redness and swelling limited to individual digits, regardless of the number of affected digits; (3) moderate redness and swelling of ankle or wrist; (4) severe redness and swelling of the entire paw including digits; (5) maximally inflamed limb with involvement of multiple joints. For example, in some cases, an antibody herein also reduces NETosis and METosis as assessed by SG+ MPO+ neutrophils, and SG+ MPO+ monocytes/macrophages, respectively, and/or reduces soluble markers of monocytes/macrophages, such as elastase and MPO, and or reduces the proportion of citrulline in H3 protein in neutrophils, monocytes, and/or macrophages. (See Example 24.)

In some embodiments, the antibody inhibits citrullination of one or more of proteoglycan 4 (PRG4), fibrinogen A (FGA), Inter-alpha-trypsin inhibitor heavy chain H4 (ITIH4), Alpha-1-microglobulin/bikunin precursor (AMBP) and gelsolin (GSN) in serum both in the presence and absence of disease-related anti-PAD4 antibodies, for example, as described in Example 27. In some such cases, the presence of disease-related anti-PAD4 antibodies in a sample from an RA patient does not significantly affect the inhibitory activity of the antibody, i.e., when compared to the inhibitory activity of the antibody in a sample from a normal, healthy subject. In some embodiments, the antibody does not cross-react with normal human tissue and does not bind to membranes of normal human tissue cells when incubated with human tissue samples in vitro at 1-5 μg/mL, such as in an assay as described in Example 28. In some embodiments, the antibody does not induce phagocytosis by neutrophils in whole blood after incubation followed by incubation with opsonized conjugated E. coli particles, for instance, such as in an assay as described in Example 29. In some embodiments, the antibody does not induce respiratory burst in neutrophils at concentrations up to 400 μg/mL, such as in an assay as described in Example 29.

### V. Exemplary Anti-PAD4 Antibodies

A. Antibody hz13-5 D31E

In some embodiments, the disclosure relates to an isolated antibody that specifically binds to protein arginine deiminase 4 (PAD4), wherein the antibody comprises (i) a heavy chain variable region (VH) comprising a heavy chain complementarity determining region 1 (HCDR1) comprising the amino acid sequence of SEQ ID NO: 62 and an HCDR3 comprising the amino acid sequence of SEQ ID NO: 6 and (ii) a light chain variable region (VL) comprising a light chain complementarity determining region 1 (LCDR1) comprising the amino acid sequence of SEQ ID NO: 7. In some embodiments, the VH comprises an HCDR2 comprising the amino acid sequence of SEQ ID NO: 5 and the VL comprises an LCDR2 comprising the amino acid sequence of SEQ ID NO: 8 and an LCDR3 comprising the amino acid sequence of SEQ ID NO: 9. In some embodiments, the antibody comprises a VH that is at least 90%, at least 95%, at least 97%, or at least 99% identical to the amino acid sequence of SEQ ID NO: 68. In some embodiments, the antibody comprises a VL that is at least 90%, at least 95%, at least 97%, or at least 99% identical to the amino acid sequence of SEQ ID NO: 70. In some embodiments, the antibody comprises both a VH that is at least 90%, at least 95%, at least 97%, or at least 99% identical to the amino acid sequence of SEQ ID NO: 68; and a VL that is at least 90%, at least 95%, at least 97%, or at least 99% identical to the amino acid sequence of SEQ ID NO: 70. In some embodiments, the VH is at least 90% identical to the amino acid sequence of SEQ ID NO: 68 and the VL is at least 90% identical to the amino acid sequence of SEQ ID NO: 70. In some embodiments, the VH is at least 95% identical to the amino acid sequence of SEQ ID NO: 68 and the VL is at least 95% identical to the amino acid sequence of SEQ ID NO: 70. In some embodiments, the VH is at least 97% identical to the amino acid sequence of SEQ ID NO: 68 and the VL is at least 97% identical to the amino acid sequence of SEQ ID NO: 70. In some embodiments, the antibody comprises a VH comprising the amino acid sequence of SEQ ID No: 68 modified by 1-10 amino acid substitutions, 1-5 amino acid substitutions, or 1-3 amino acid substitutions. In some embodiments, the antibody comprises a VL comprising the amino acid sequence of SEQ ID No: 70 modified by 1-10 amino acid substitutions, 1-5 amino acid substitutions, or 1-3 amino acid substitutions. In some embodiments, the antibody comprises a both a VH comprising the amino acid sequence of SEQ ID No: 68 modified by 1-10 amino acid substitutions, 1-5 amino acid substitutions, or 1-3 amino acid substitutions and a VL comprising the amino acid sequence of SEQ ID No: 70 modified by 1-10 amino acid substitutions, 1-5 amino acid substitutions, or 1-3 amino acid substitutions. In some embodiments, the VH of the antibody comprises a glycine at Kabat position 94 (corresponding to position 98 of SEQ ID NO: 10).

In some embodiments, the antibody comprises a VH comprising the amino acid sequence of SEQ ID No: 68. In some embodiments, the antibody comprises a VH comprising the amino acid sequence of SEQ ID No: 68; and a VL that is at least 90%, at least 95%, at least 97%, or at least 99% identical to the amino acid sequence of SEQ ID NO: 70. In some embodiments, the antibody comprises a VL comprising the amino acid sequence of SEQ ID No: 70. In some embodiments, the antibody comprises a VH that is at least 90%, at least 95%, at least 97%, or at least 99% identical to the amino acid sequence of SEQ ID NO: 68; and a VL comprising the amino acid sequence of SEQ ID No: 70. In some embodiments, the antibody comprises a VH comprising the amino acid sequence of SEQ ID No: 68 and a VL comprising the amino acid sequence of SEQ ID No: 70.

In some cases, the antibody is an IgG antibody, such as a human IgG1, IgG2, IgG3, or IgG4 antibody. In some embodiments, the antibody comprises a human IgG1 heavy chain constant region comprising an amino acid sequence at least 90%, at least 95%, at least 97%, or at least 99% identical to the amino acid sequence of any one of SEQ ID Nos: 174, 176, 178, 180, 182, 184, 186, 188, 190, or 192. In some embodiments, the antibody comprises a light chain constant region comprising an amino acid sequence at least 90%, at least 95%, at least 97%, or at least 99% identical to the amino acid sequence of SEQ ID NO: 194. In some embodiments, the antibody comprises both a human IgG1 heavy chain constant region comprising an amino acid sequence at least 90%, at least 95%, at least 97%, or at least 99% identical to the amino acid sequence of any one of SEQ ID Nos: 174, 176, 178, 180, 182, 184, 186, 188, 190, or 192; and a light chain constant region comprising an amino acid sequence at least 90%, at least 95%, at least 97%, or at least 99% identical to the amino acid sequence of SEQ ID NO: 194. In some cases, the antibody comprises a heavy chain constant region comprising an amino acid sequence modified by 1-10 amino acid substitutions, 1-5 amino acid substitutions, or 1-3 amino acid substitutions compared to the amino acid sequence of any one of SEQ ID Nos: 174, 176, 178, 180, 182, 184, 186, 188, 190, or 192. In some cases, the antibody comprises a light chain constant region comprising an amino acid sequence modified by 1-10 amino acid substitutions, 1-5 amino acid substitutions, or 1-3 amino acid substitutions compared to the amino acid sequence of SEQ ID NO: 194. In some cases, the antibody comprises both a heavy chain constant region comprising an amino acid sequence modified by 1-10 amino acid substitutions, 1-5 amino acid substitutions, or 1-3 amino acid substitutions compared to the amino acid sequence of any one of SEQ ID Nos: 174, 176, 178, 180, 182, 184, 186, 188, 190, or 192; and a light chain constant region comprising an amino acid sequence modified by 1-10 amino acid substitutions, 1-5 amino acid substitutions, or 1-3 amino acid substitutions compared to the amino acid sequence of SEQ ID NO: 194. In some embodiments, the antibody comprises a human IgG1 heavy chain constant region comprising amino acid sequence of any one of SEQ ID Nos: 174, 176, 178, 180, 182, 184, 186, 188, 190, or 192. In some embodiments, the antibody comprises a light chain constant region comprising the amino acid sequence of SEQ ID NO: 194. In some embodiments, the antibody comprises both a human IgG1 heavy chain constant region comprising the amino acid sequence of any one of SEQ ID Nos: 174, 176, 178, 180, 182, 184, 186, 188, 190, or 192; and a light chain constant region comprising the amino acid sequence of SEQ ID NO: 194.

In some cases, the antibody comprises a heavy chain (HC) comprising the amino acid sequence of SEQ ID NO: 68 followed by the amino acid sequence of SEQ ID NO: 178 and a light chain (LC) comprising the amino acid sequence of SEQ ID NO: 70 followed by the amino acid sequence of SEQ ID NO: 194. In some cases, the antibody comprises a heavy chain (HC) comprising the amino acid sequence of SEQ ID NO: 68 followed by the amino acid sequence of SEQ ID NO: 180 and a light chain (LC) comprising the amino acid sequence of SEQ ID NO: 70 followed by the amino acid sequence of SEQ ID NO: 194. In some cases, the antibody comprises an HC comprising the amino acid sequence of SEQ ID NO: 196 and a light chain comprising the amino acid sequence of SEQ ID NO: 200. In some cases, the antibody comprises an HC comprising the amino acid sequence of SEQ ID NO: 198 and a light chain comprising the amino acid sequence of SEQ ID NO: 200. In some cases, the antibody comprises an HC consisting of the amino acid sequence of SEQ ID NO: 196 and a light chain consisting of the amino acid sequence of SEQ ID NO: 200. In some cases, the antibody comprises an HC consisting of the amino acid sequence of SEQ ID NO: 198 and a light chain consisting of the amino acid sequence of SEQ ID NO: 200.

In some cases the antibody binds to an epitope on PAD4 comprising SEQ ID NO: 217 and SEQ ID NO: 218.

In some embodiments, the antibody specifically binds to PAD4 in an SPR assay with a K_{D} from 0.05 nM to 1 nM, from 0.1 nM to 1 nM, from 0.1 nM to 0.5 nM, from 0.05 nM to 0.5 nM, or from 0.05 nM to 0.1 nM, or of 0.09 nM, 0.1 nM, or 0.2 nM, for example, both in the presence of 1-2 mM calcium chloride (e.g., 1 mM calcium chloride) and in the absence of calcium ion (due to absence of added calcium salt as well as the presence of EDTA, such as 1 mM or 2 mM EDTA). See Tables 2 and 3. In some embodiments, the antibody binds specifically to human PAD4 but not to cynomolgus or murine PAD4 by SPR.

In some embodiments, the antibody has an immunogenicity score of 0-1%, 0-0.5%, or 0%, 0.5%, or 1% as measured using an Epivax® immunogenicity test. In some cases, the antibody has a score of 0%. (See Table 10.) In some cases, the antibody is less immunogenic that one, two, or all three of Campath® (alemtuzumab), Rituxan® (rituximab), and Zenapax® (daclizumab), as measured in an in silico immunogenicity assay.

In some embodiments, the antibody inhibits PAD4 conversion of arginine in peptide substrate (SHQESTRGKSKGKAAAAA; SEQ ID NO: 232) to citrulline in vitro with an IC50 of 0.1-10 nM, such as 0.2-5 nM, optionally in a dose-dependent manner with a PAD4 concentration of 1-8 μg/mL. (See, for example, Example 16 below.)

In some embodiments, the antibody inhibits the enzymatic activity of PAD4 in vitro in the presence of an antibody that binds and activates human PAD4 (an activating antibody). In some embodiments, the activating antibody is an antibody derived from a patient with rheumatoid arthritis. In some embodiments, the activating antibody is an antibody that is cross reactive for human PAD3 and human PAD4.

In some cases, the antibody reduces the amount of extracellular citrullinated histone H3 in LPS-stimulated human blood monocytes compared to an isotype control antibody. In some embodiments, the LPS-stimulated human blood monocytes are CD14+CD16-monocytes isolated from fresh human PBMCs. (See Example 17 herein.) In some embodiments, the antibody is capable of being internalized by LPS-stimulated human blood monocytes, such as by LPS-stimulated CD14+ human monocytes. (See Example 18.)

In some embodiments, the antibody inhibits PAD4 function in an inflamed lung. For example, this may be demonstrated by decreased citrullination of histone H3 or ITIH4 in broncheoalveolar lavage fluid (BALF) collected from the lung, as described in Example 25 herein. For example, BALF may be collected from mice with acute or chronic lung inflammation and assayed for the amount of citrullinated H3 or ITIH4 protein in the presence of the antibody and in the presence of a wild-type control. In some embodiments, the mice are human PAD4 knock-in mice. In some cases, the amount of citrullinated H3 is reduced by at least 15%, at least 30%, at least 40%, at least 50%, or at least 60%, and/or the amount of citrullinated ITIH4 is reduced by at least 50%, at least 70%, or at least 80% in the presence of the antibody in human PAD4 knock-in mice, compared to an isotope control (IC) antibody, for example, as calculated by the following formula: % inhibition=[(IC−naïve)−(PAD4 mAb−naïve)/(IC−naïve)]×100, in which the amount of citrullinated H3 or ITIH4 is measured for untreated (naïve) mice, mice receiving IC, and mice receiving an anti-PAD4 antibody.

In some embodiments, the antibody inhibits PAD4 function in an inflamed joint, as described in Example 26. This may be demonstrated, for example, by decreased citrullination of ITIH4 and/or PRG4 in joint tissue in a mouse model with acute or chronic joint inflammation, such as induced by LPS injection of a joint. In some cases, the mice may be human PAD4 knock-in mice. In some cases, the antibody reduces the amount of extracellular citrullinated PRG4 and/or citrullinated ITIH4 in mouse patella in an LPS-induced acute joint injury model with human PAD4 knock-in mice compared to an isotype control antibody. In some cases, the EC50 of reduction of citrullinated ITIH4 and/or citrullinated PRG4 is 2 nM or lower, 1 nM or lower, 0.1 nM or lower, 0.05-2 nM, or 0.1-1 nM.

In some embodiments, the antibody may be useful in inhibiting citrullination of proteins either in a subject, or in vitro, such as in a biological sample. For instance, in some embodiments, the antibody inhibits citrullination of one or more of proteoglycan 4 (PRG4), fibrinogen A (FGA), Inter-alpha-trypsin inhibitor heavy chain H4 (ITIH4), alpha-1-microglobulin/bikunin precursor (AMBP) and gelsolin (GSN) in serum both in the presence and absence of disease-related anti-PAD4 antibodies, for example, as described in Example 27. In some such cases, the presence of disease-related anti-PAD4 antibodies in a sample from an RA patient does not significantly affect the inhibitory activity of the antibody, for instance, when compared to the inhibitory activity of the antibody in a sample from a normal, healthy subject. In some embodiments, the antibody does not cross-react with normal human tissue and does not bind to membranes of normal human tissue cells when incubated with human tissue samples in vitro at 1-5 μg/mL, such as in an assay as described in Example 28. In some embodiments, the antibody does not induce phagocytosis by neutrophils in whole blood after incubation followed by incubation with opsonized conjugated E. coli particles, for instance, such as in an assay as described in Example 29. In some embodiments, the antibody does not induce respiratory burst in neutrophils at concentrations up to 400 μg/mL, such as in an assay as described in Example 29.

B. Antibody hz13-5

In some embodiments, the disclosure relates to an isolated antibody that specifically binds to protein arginine deiminase 4 (PAD4), wherein the antibody comprises (i) a heavy chain variable region (VH) comprising a heavy chain complementarity determining region 1 (HCDR1) comprising the amino acid sequence of SEQ ID NO: 4 and an HCDR3 comprising the amino acid sequence of SEQ ID NO: 6 and (ii) a light chain variable region (VL) comprising a light chain complementarity determining region 1 (LCDR1) comprising the amino acid sequence of SEQ ID NO: 7. In some embodiments, the VH comprises an HCDR2 comprising the amino acid sequence of SEQ ID NO: 5 and the VL comprises an LCDR2 comprising the amino acid sequence of SEQ ID NO: 8 and an LCDR3 comprising the amino acid sequence of SEQ ID NO: 9. In some embodiments, the antibody comprises a VH that is at least 90%, at least 95%, at least 97%, or at least 99% identical to the amino acid sequence of SEQ ID NO: 30. In some embodiments, the antibody comprises a VL that is at least 90%, at least 95%, at least 97%, or at least 99% identical to the amino acid sequence of SEQ ID NO: 32. In some embodiments, the antibody comprises both a VH that is at least 90%, at least 95%, at least 97%, or at least 99% identical to the amino acid sequence of SEQ ID NO: 30; and a VL that is at least 90%, at least 95%, at least 97%, or at least 99% identical to the amino acid sequence of SEQ ID NO: 32. In some embodiments, the VH is at least 90% identical to the amino acid sequence of SEQ ID NO: 30 and the VL is at least 90% identical to the amino acid sequence of SEQ ID NO: 32. In some embodiments, the VH is at least 95% identical to the amino acid sequence of SEQ ID NO: 30 and the VL is at least 95% identical to the amino acid sequence of SEQ ID NO: 32. In some embodiments, the VH is at least 97% identical to the amino acid sequence of SEQ ID NO: 30 and the VL is at least 97% identical to the amino acid sequence of SEQ ID NO: 32. In some embodiments, the antibody comprises a VH comprising the amino acid sequence of SEQ ID No: 30 modified by 1-10 amino acid substitutions, 1-5 amino acid substitutions, or 1-3 amino acid substitutions. In some embodiments, the antibody comprises a VL comprising the amino acid sequence of SEQ ID No: 32 modified by 1-10 amino acid substitutions, 1-5 amino acid substitutions, or 1-3 amino acid substitutions. In some embodiments, the antibody comprises a both a VH comprising the amino acid sequence of SEQ ID No: 30 modified by 1-10 amino acid substitutions, 1-5 amino acid substitutions, or 1-3 amino acid substitutions and a VL comprising the amino acid sequence of SEQ ID No: 32 modified by 1-10 amino acid substitutions, 1-5 amino acid substitutions, or 1-3 amino acid substitutions. In some embodiments, the VH of the antibody comprises a glycine at Kabat position 94 (corresponding to position 98 of SEQ ID NO: 10).

In some embodiments, the antibody comprises a VH comprising the amino acid sequence of SEQ ID No: 30. In some embodiments, the antibody comprises a VH comprising the amino acid sequence of SEQ ID No: 30; and a VL that is at least 90%, at least 95%, at least 97%, or at least 99% identical to the amino acid sequence of SEQ ID NO: 32. In some embodiments, the antibody comprises a VL comprising the amino acid sequence of SEQ ID No: 32. In some embodiments, the antibody comprises a VH that is at least 90%, at least 95%, at least 97%, or at least 99% identical to the amino acid sequence of SEQ ID NO: 30; and a VL comprising the amino acid sequence of SEQ ID No: 32. In some embodiments, the antibody comprises a VH comprising the amino acid sequence of SEQ ID No: 30 and a VL comprising the amino acid sequence of SEQ ID No: 32.

In some cases, the antibody is an IgG antibody, such as a human IgG1, IgG2, IgG3, or IgG4 antibody. In some embodiments, the antibody comprises a human IgG1 heavy chain constant region comprising an amino acid sequence at least 90%, at least 95%, at least 97%, or at least 99% identical to the amino acid sequence of any one of SEQ ID Nos: 174, 176, 178, 180, 182, 184, 186, 188, 190, or 192. In some embodiments, the antibody comprises a light chain constant region comprising an amino acid sequence at least 90%, at least 95%, at least 97%, or at least 99% identical to the amino acid sequence of SEQ ID NO: 194. In some embodiments, the antibody comprises both a human IgG1 heavy chain constant region comprising an amino acid sequence at least 90%, at least 95%, at least 97%, or at least 99% identical to the amino acid sequence of any one of SEQ ID Nos: 174, 176, 178, 180, 182, 184, 186, 188, 190, or 192; and a light chain constant region comprising an amino acid sequence at least 90%, at least 95%, at least 97%, or at least 99% identical to the amino acid sequence of SEQ ID NO: 194. In some cases, the antibody comprises a heavy chain constant region comprising an amino acid sequence modified by 1-10 amino acid substitutions, 1-5 amino acid substitutions, or 1-3 amino acid substitutions compared to the amino acid sequence of any one of SEQ ID Nos: 174, 176, 178, 180, 182, 184, 186, 188, 190, or 192. In some cases, the antibody comprises a light chain constant region comprising an amino acid sequence modified by 1-10 amino acid substitutions, 1-5 amino acid substitutions, or 1-3 amino acid substitutions compared to the amino acid sequence of SEQ ID NO: 194. In some cases, the antibody comprises both a heavy chain constant region comprising an amino acid sequence modified by 1-10 amino acid substitutions, 1-5 amino acid substitutions, or 1-3 amino acid substitutions compared to the amino acid sequence of any one of SEQ ID Nos: 174, 176, 178, 180, 182, 184, 186, 188, 190, or 192; and a light chain constant region comprising an amino acid sequence modified by 1-10 amino acid substitutions, 1-5 amino acid substitutions, or 1-3 amino acid substitutions compared to the amino acid sequence of SEQ ID NO: 194. In some embodiments, the antibody comprises a human IgG1 heavy chain constant region comprising amino acid sequence of any one of SEQ ID Nos: 174, 176, 178, 180, 182, 184, 186, 188, 190, or 192. In some embodiments, the antibody comprises a light chain constant region comprising the amino acid sequence of SEQ ID NO: 194. In some embodiments, the antibody comprises both a human IgG1 heavy chain constant region comprising the amino acid sequence of any one of SEQ ID Nos: 174, 176, 178, 180, 182, 184, 186, 188, 190, or 192; and a light chain constant region comprising the amino acid sequence of SEQ ID NO: 194.

In some cases, the antibody comprises a heavy chain (HC) comprising the amino acid sequence of SEQ ID NO: 30 followed by the amino acid sequence of SEQ ID NO: 178 and a light chain (LC) comprising the amino acid sequence of SEQ ID NO: 32 followed by the amino acid sequence of SEQ ID NO: 194. In some cases, the antibody comprises a heavy chain (HC) comprising the amino acid sequence of SEQ ID NO: 30 followed by the amino acid sequence of SEQ ID NO: 180 and a light chain (LC) comprising the amino acid sequence of SEQ ID NO: 32 followed by the amino acid sequence of SEQ ID NO: 194.

In some cases the antibody binds to an epitope on PAD4 comprising SEQ ID NO: 217 and SEQ ID NO: 218.

In some embodiments, the antibody specifically binds to PAD4 in an SPR assay with a K_{D} from 0.05 nM to 1 nM, from 0.1 nM to 1 nM, from 0.1 nM to 0.5 nM, from 0.05 nM to 0.5 nM, or from 0.05 nM to 0.1 nM, or of 0.09 nM, 0.1 nM, or 0.2 nM, for example, both in the presence of 1-2 mM calcium chloride (e.g., 1 mM calcium chloride) and in the absence of calcium ion (due to absence of added calcium salt as well as the presence of EDTA, such as 1 mM or 2 mM EDTA). See Tables 2 and 3. In some embodiments, the antibody binds specifically to human PAD4 but not to cynomolgus or murine PAD4 by SPR.

In some embodiments, the antibody has an ECM score of less than 10, less than 5, 1-5, 1-2, or 1. (See, for example Table 5.)

In some embodiments, the antibody has an immunogenicity score of 0-1%, 0-0.5%, or 0%, 0.5%, or 1% as measured using an Epivax® immunogenicity test. (See Table 10.) In some cases, the antibody is less immunogenic that one, two, or all three of Campath® (alemtuzumab), Rituxan® (rituximab), and Zenapax® (daclizumab), as measured in an in silico immunogenicity assay.

In some embodiments, the antibody inhibits PAD4 conversion of arginine in peptide substrate (TSTGGRQGSHH; SEQ ID NO: 216) to citrulline in vitro with an IC50 of 25-100 nM or 30-60 nM or 40-60 nM, for example, in an assay as shown in Example 4 below.

In some embodiments, the antibody inhibits the enzymatic activity of PAD4 in vitro in the presence of an antibody that binds and activates human PAD4 (an activating antibody). In some embodiments, the activating antibody is an antibody derived from a patient with rheumatoid arthritis. In some embodiments, the activating antibody is an antibody that is cross reactive for human PAD3 and human PAD4.

In some cases, the antibody reduces the amount of extracellular citrullinated histone H3 in LPS-stimulated human blood monocytes compared to an isotype control antibody. In some embodiments, the LPS-stimulated human blood monocytes are CD14+CD16− monocytes isolated from fresh human PBMCs. (See Example 17 herein.) In some embodiments, the antibody is capable of being internalized by LPS-stimulated human blood monocytes, such as by LPS-stimulated CD14+ human monocytes. (See Example 18.)

In some embodiments, the antibody inhibits PAD4 function in an inflamed lung. For example, this may be demonstrated by decreased citrullination of histone H3 or ITIH4 in broncheoalveolar lavage fluid (BALF) collected from the lung, as described in Example 25 herein. For example, BALF may be collected from mice with acute or chronic lung inflammation and assayed for the amount of citrullinated H3 or ITIH4 protein in the presence of the antibody and in the presence of a wild-type control. In some embodiments, the mice are human PAD4 knock-in mice. In some cases, the amount of citrullinated H3 is reduced by at least 15%, at least 30%, at least 40%, at least 50%, or at least 60%, and/or the amount of citrullinated ITIH4 is reduced by at least 50%, at least 70%, or at least 80% in the presence of the antibody in human PAD4 knock-in mice, compared to an isotope control (IC) antibody, for example, as calculated by the following formula: % inhibition=[(IC−na-ve)−(PAD4 mAb−naïve)/(IC−naïve)]×100, in which the amount of citrullinated H3 or ITIH4 is measured for untreated (naïve) mice, mice receiving IC, and mice receiving an anti-PAD4 antibody.

In some embodiments, the antibody inhibits PAD4 function in an inflamed joint, as described in Example 26. This may be demonstrated, for example, by decreased citrullination of ITIH4 and/or PRG4 in joint tissue in a mouse model with acute or chronic joint inflammation, such as induced by LPS injection of a joint. In some cases, the mice may be human PAD4 knock-in mice. In some cases, the antibody reduces the amount of extracellular citrullinated PRG4 and/or citrullinated ITIH4 in mouse patella in an LPS-induced acute joint injury model with human PAD4 knock-in mice compared to an isotype control antibody. In some cases, the EC50 of reduction of citrullinated ITIH4 and/or citrullinated PRG4 is 2 nM or lower, 1 nM or lower, 0.1 nM or lower, 0.05-2 nM, or 0.1-1 nM.

C. Antibody hz13-12

In some embodiments, the disclosure relates to an isolated antibody that specifically binds to protein arginine deiminase 4 (PAD4), wherein the antibody comprises (i) a heavy chain variable region (VH) comprising a heavy chain complementarity determining region 1 (HCDR1) comprising the amino acid sequence of SEQ ID NO: 62 and an HCDR3 comprising the amino acid sequence of SEQ ID NO: 6 and (ii) a light chain variable region (VL) comprising a light chain complementarity determining region 1 (LCDR1) comprising the amino acid sequence of SEQ ID NO: 7. In some embodiments, the VH comprises an HCDR2 comprising the amino acid sequence of SEQ ID NO: 5 and the VL comprises an LCDR2 comprising the amino acid sequence of SEQ ID NO: 8 and an LCDR3 comprising the amino acid sequence of SEQ ID NO: 9. In some embodiments, the antibody comprises a VH that is at least 90%, at least 95%, at least 97%, or at least 99% identical to the amino acid sequence of SEQ ID NO: 58. In some embodiments, the antibody comprises a VL that is at least 90%, at least 95%, at least 97%, or at least 99% identical to the amino acid sequence of SEQ ID NO: 60. In some embodiments, the antibody comprises both a VH that is at least 90%, at least 95%, at least 97%, or at least 99% identical to the amino acid sequence of SEQ ID NO: 58; and a VL that is at least 90%, at least 95%, at least 97%, or at least 99% identical to the amino acid sequence of SEQ ID NO: 60. In some embodiments, the VH is at least 90% identical to the amino acid sequence of SEQ ID NO: 58 and the VL is at least 90% identical to the amino acid sequence of SEQ ID NO: 60. In some embodiments, the VH is at least 95% identical to the amino acid sequence of SEQ ID NO: 58 and the VL is at least 95% identical to the amino acid sequence of SEQ ID NO: 60. In some embodiments, the VH is at least 97% identical to the amino acid sequence of SEQ ID NO: 58 and the VL is at least 97% identical to the amino acid sequence of SEQ ID NO: 60. In some embodiments, the antibody comprises a VH comprising the amino acid sequence of SEQ ID No: 58 modified by 1-10 amino acid substitutions, 1-5 amino acid substitutions, or 1-3 amino acid substitutions. In some embodiments, the antibody comprises a VL comprising the amino acid sequence of SEQ ID No: 60 modified by 1-10 amino acid substitutions, 1-5 amino acid substitutions, or 1-3 amino acid substitutions. In some embodiments, the antibody comprises both a VH comprising the amino acid sequence of SEQ ID No: 58 modified by 1-10 amino acid substitutions, 1-5 amino acid substitutions, or 1-3 amino acid substitutions and a VL comprising the amino acid sequence of SEQ ID No: 60 modified by 1-10 amino acid substitutions, 1-5 amino acid substitutions, or 1-3 amino acid substitutions. In some embodiments, the VH of the antibody comprises a glycine at Kabat position 94 (corresponding to position 98 of SEQ ID NO: 10).

In some embodiments, the antibody comprises a VH comprising the amino acid sequence of SEQ ID No: 58. In some embodiments, the antibody comprises a VH comprising the amino acid sequence of SEQ ID No: 58; and a VL that is at least 90%, at least 95%, at least 97%, or at least 99% identical to the amino acid sequence of SEQ ID NO: 60. In some embodiments, the antibody comprises a VL comprising the amino acid sequence of SEQ ID No: 60. In some embodiments, the antibody comprises a VH that is at least 90%, at least 95%, at least 97%, or at least 99% identical to the amino acid sequence of SEQ ID NO: 58; and a VL comprising the amino acid sequence of SEQ ID No: 60. In some embodiments, the antibody comprises a VH comprising the amino acid sequence of SEQ ID No: 58 and a VL comprising the amino acid sequence of SEQ ID No: 60.

In some cases, the antibody is an IgG antibody, such as a human IgG1, IgG2, IgG3, or IgG4 antibody. In some embodiments, the antibody comprises a human IgG1 heavy chain constant region comprising an amino acid sequence at least 90%, at least 95%, at least 97%, or at least 99% identical to the amino acid sequence of any one of SEQ ID Nos: 174, 176, 178, 180, 182, 184, 186, 188, 190, or 192. In some embodiments, the antibody comprises a light chain constant region comprising an amino acid sequence at least 90%, at least 95%, at least 97%, or at least 99% identical to the amino acid sequence of SEQ ID NO: 194. In some embodiments, the antibody comprises both a human IgG1 heavy chain constant region comprising an amino acid sequence at least 90%, at least 95%, at least 97%, or at least 99% identical to the amino acid sequence of any one of SEQ ID Nos: 174, 176, 178, 180, 182, 184, 186, 188, 190, or 192; and a light chain constant region comprising an amino acid sequence at least 90%, at least 95%, at least 97%, or at least 99% identical to the amino acid sequence of SEQ ID NO: 194. In some cases, the antibody comprises a heavy chain constant region comprising an amino acid sequence modified by 1-10 amino acid substitutions, 1-5 amino acid substitutions, or 1-3 amino acid substitutions compared to the amino acid sequence of any one of SEQ ID Nos: 174, 176, 178, 180, 182, 184, 186, 188, 190, or 192. In some cases, the antibody comprises a light chain constant region comprising an amino acid sequence modified by 1-10 amino acid substitutions, 1-5 amino acid substitutions, or 1-3 amino acid substitutions compared to the amino acid sequence of SEQ ID NO: 194. In some cases, the antibody comprises both a heavy chain constant region comprising an amino acid sequence modified by 1-10 amino acid substitutions, 1-5 amino acid substitutions, or 1-3 amino acid substitutions compared to the amino acid sequence of any one of SEQ ID Nos: 174, 176, 178, 180, 182, 184, 186, 188, 190, or 192; and a light chain constant region comprising an amino acid sequence modified by 1-10 amino acid substitutions, 1-5 amino acid substitutions, or 1-3 amino acid substitutions compared to the amino acid sequence of SEQ ID NO: 194. In some embodiments, the antibody comprises a human IgG1 heavy chain constant region comprising amino acid sequence of any one of SEQ ID Nos: 174, 176, 178, 180, 182, 184, 186, 188, 190, or 192. In some embodiments, the antibody comprises a light chain constant region comprising the amino acid sequence of SEQ ID NO: 194. In some embodiments, the antibody comprises both a human IgG1 heavy chain constant region comprising the amino acid sequence of any one of SEQ ID Nos: 174, 176, 178, 180, 182, 184, 186, 188, 190, or 192; and a light chain constant region comprising the amino acid sequence of SEQ ID NO: 194.

In some cases, the antibody comprises a heavy chain (HC) comprising the amino acid sequence of SEQ ID NO: 58 followed by the amino acid sequence of SEQ ID NO: 178 and a light chain (LC) comprising the amino acid sequence of SEQ ID NO: 60 followed by the amino acid sequence of SEQ ID NO: 194. In some cases, the antibody comprises a heavy chain (HC) comprising the amino acid sequence of SEQ ID NO: 58 followed by the amino acid sequence of SEQ ID NO: 180 and a light chain (LC) comprising the amino acid sequence of SEQ ID NO: 60 followed by the amino acid sequence of SEQ ID NO: 194.

In some cases the antibody binds to an epitope on PAD4 comprising SEQ ID NO: 217 and SEQ ID NO: 218.

In some embodiments, the antibody specifically binds to PAD4 in an SPR assay with a K_{D} from 0.05 nM to 1 nM, from 0.1 nM to 1 nM, from 0.1 nM to 0.5 nM, from 0.05 nM to 0.5 nM, or from 0.05 nM to 0.1 nM, or of 0.09 nM, 0.1 nM, or 0.2 nM, for example, both in the presence of 1-2 mM calcium chloride (e.g., 1 mM calcium chloride) and in the absence of calcium ion (due to absence of added calcium salt as well as the presence of EDTA, such as 1 mM or 2 mM EDTA). See Tables 2 and 3. In some embodiments, the antibody binds specifically to human PAD4 but not to cynomolgus or murine PAD4 by SPR.

In some embodiments, the antibody has an ECM score of less than 10, less than 5, 1-5, 1-2, or 1. (See, for example Table 5.)

In some embodiments, the antibody has an immunogenicity score of 0-1%, 0-0.5%, or 0%, 0.5%, or 1% as measured using an Epivax® immunogenicity test. In some cases, the antibody has a score of 0%. (See Table 10.) In some cases, the antibody is less immunogenic that one, two, or all three of Campath® (alemtuzumab), Rituxan® (rituximab), and Zenapax® (daclizumab), as measured in an in silico immunogenicity assay.

In some embodiments, the antibody inhibits PAD4 conversion of arginine in peptide substrate (TSTGGRQGSHH; SEQ ID NO: 216) to citrulline in vitro with an IC50 of 25-100 nM or 30-60 nM or 40-60 nM, for example, in an assay as shown in Example 4 below.

In some embodiments, the antibody inhibits the enzymatic activity of PAD4 in vitro in the presence of an antibody that binds and activates human PAD4 (an activating antibody). In some embodiments, the activating antibody is an antibody derived from a patient with rheumatoid arthritis. In some embodiments, the activating antibody is an antibody that is cross reactive for human PAD3 and human PAD4.

In some cases, the antibody reduces the amount of extracellular citrullinated histone H3 in LPS-stimulated human blood monocytes compared to an isotype control antibody. In some embodiments, the LPS-stimulated human blood monocytes are CD14+CD16-monocytes isolated from fresh human PBMCs. (See Example 17 herein.) In some embodiments, the antibody is capable of being internalized by LPS-stimulated human blood monocytes, such as by LPS-stimulated CD14+ human monocytes. (See Example 18.)

In some embodiments, the antibody inhibits PAD4 function in an inflamed lung. For example, this may be demonstrated by decreased citrullination of histone H3 or ITIH4 in broncheoalveolar lavage fluid (BALF) collected from the lung, as described in Example 25 herein. For example, BALF may be collected from mice with acute or chronic lung inflammation and assayed for the amount of citrullinated H3 or ITIH4 protein in the presence of the antibody and in the presence of a wild-type control. In some embodiments, the mice are human PAD4 knock-in mice. In some cases, the amount of citrullinated H3 is reduced by at least 15%, at least 30%, at least 40%, at least 50%, or at least 60%, and/or the amount of citrullinated ITIH4 is reduced by at least 50%, at least 70%, or at least 80% in the presence of the antibody in human PAD4 knock-in mice, compared to an isotope control (IC) antibody, for example, as calculated by the following formula: % inhibition=[(IC−na-ve)−(PAD4 mAb−naïve)/(IC−naïve)]×100, in which the amount of citrullinated H3 or ITIH4 is measured for untreated (naïve) mice, mice receiving IC, and mice receiving an anti-PAD4 antibody.

In some embodiments, the antibody inhibits PAD4 function in an inflamed joint, as described in Example 26. This may be demonstrated, for example, by decreased citrullination of ITIH4 and/or PRG4 in joint tissue in a mouse model with acute or chronic joint inflammation, such as induced by LPS injection of a joint. In some cases, the mice may be human PAD4 knock-in mice. In some cases, the antibody reduces the amount of extracellular citrullinated PRG4 and/or citrullinated ITIH4 in mouse patella in an LPS-induced acute joint injury model with human PAD4 knock-in mice compared to an isotype control antibody. In some cases, the EC50 of reduction of citrullinated ITIH4 and/or citrullinated PRG4 is 2 nM or lower, 1 nM or lower, 0.1 nM or lower, 0.05-2 nM, or 0.1-1 nM.

D. Antibody hz20-2

In some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of SEQ ID NO: 72, an HCDR2 comprising the amino acid sequence of SEQ ID NO: 73, and an HCDR3 comprising the amino acid sequence of SEQ ID NO: 74; and a light chain variable region (VL) comprising a light chain complementarity determining region 1 (LCDR1) comprising the amino acid sequence of SEQ ID NO: 75, an LCDR2 comprising the amino acid sequence of SEQ ID NO: 76, and an LCDR3 comprising the amino acid sequence of SEQ ID NO: 77. In some embodiments, the antibody comprises a VH that is at least 90%, at least 95%, at least 97%, or at least 99% identical to the amino acid sequence of SEQ ID NO: 86. In some embodiments, the antibody comprises a VL that is at least 90%, at least 95%, at least 97%, or at least 99% identical to the amino acid sequence of SEQ ID NO: 88. In some embodiments, the antibody comprises both a VH that is at least 90%, at least 95%, at least 97%, or at least 99% identical to the amino acid sequence of SEQ ID NO: 86; and a VL that is at least 90%, at least 95%, at least 97%, or at least 99% identical to the amino acid sequence of SEQ ID NO: 88. In some embodiments, the VH is at least 90% identical to the amino acid sequence of SEQ ID NO: 86 and the VL is at least 90% identical to the amino acid sequence of SEQ ID NO: 88. In some embodiments, the VH is at least 95% identical to the amino acid sequence of SEQ ID NO: 86 and the VL is at least 95% identical to the amino acid sequence of SEQ ID NO: 88. In some embodiments, the VH is at least 97% identical to the amino acid sequence of SEQ ID NO: 86 and the VL is at least 97% identical to the amino acid sequence of SEQ ID NO: 88. In some embodiments, the antibody comprises a VH comprising the amino acid sequence of SEQ ID No: 86 modified by 1-10 amino acid substitutions, 1-5 amino acid substitutions, or 1-3 amino acid substitutions. In some embodiments, the antibody comprises a VL comprising the amino acid sequence of SEQ ID No: 88 modified by 1-10 amino acid substitutions, 1-5 amino acid substitutions, or 1-3 amino acid substitutions. In some embodiments, the antibody comprises both a VH comprising the amino acid sequence of SEQ ID No: 86 modified by 1-10 amino acid substitutions, 1-5 amino acid substitutions, or 1-3 amino acid substitutions and a VL comprising the amino acid sequence of SEQ ID No: 88 modified by 1-10 amino acid substitutions, 1-5 amino acid substitutions, or 1-3 amino acid substitutions.

In some embodiments, the antibody comprises a VH comprising the amino acid sequence of SEQ ID No: 86. In some embodiments, the antibody comprises a VH comprising the amino acid sequence of SEQ ID No: 86; and a VL that is at least 90%, at least 95%, at least 97%, or at least 99% identical to the amino acid sequence of SEQ ID NO: 88. In some embodiments, the antibody comprises a VL comprising the amino acid sequence of SEQ ID No: 88. In some embodiments, the antibody comprises a VH that is at least 90%, at least 95%, at least 97%, or at least 99% identical to the amino acid sequence of SEQ ID NO: 86; and a VL comprising the amino acid sequence of SEQ ID No: 88. In some embodiments, the antibody comprises a VH comprising the amino acid sequence of SEQ ID No: 86 and a VL comprising the amino acid sequence of SEQ ID No: 88.

In some cases, the antibody is an IgG antibody, such as a human IgG1, IgG2, IgG3, or IgG4 antibody. In some embodiments, the antibody comprises a human IgG1 heavy chain constant region comprising an amino acid sequence at least 90%, at least 95%, at least 97%, or at least 99% identical to the amino acid sequence of any one of SEQ ID Nos: 174, 176, 178, 180, 182, 184, 186, 188, 190, or 192. In some embodiments, the antibody comprises a light chain constant region comprising an amino acid sequence at least 90%, at least 95%, at least 97%, or at least 99% identical to the amino acid sequence of SEQ ID NO: 194. In some embodiments, the antibody comprises both a human IgG1 heavy chain constant region comprising an amino acid sequence at least 90%, at least 95%, at least 97%, or at least 99% identical to the amino acid sequence of any one of SEQ ID Nos: 174, 176, 178, 180, 182, 184, 186, 188, 190, or 192; and a light chain constant region comprising an amino acid sequence at least 90%, at least 95%, at least 97%, or at least 99% identical to the amino acid sequence of SEQ ID NO: 194. In some cases, the antibody comprises a heavy chain constant region comprising an amino acid sequence modified by 1-10 amino acid substitutions, 1-5 amino acid substitutions, or 1-3 amino acid substitutions compared to the amino acid sequence of any one of SEQ ID Nos: 174, 176, 178, 180, 182, 184, 186, 188, 190, or 192. In some cases, the antibody comprises a light chain constant region comprising an amino acid sequence modified by 1-10 amino acid substitutions, 1-5 amino acid substitutions, or 1-3 amino acid substitutions compared to the amino acid sequence of SEQ ID NO: 194. In some cases, the antibody comprises both a heavy chain constant region comprising an amino acid sequence modified by 1-10 amino acid substitutions, 1-5 amino acid substitutions, or 1-3 amino acid substitutions compared to the amino acid sequence of any one of SEQ ID Nos: 174, 176, 178, 180, 182, 184, 186, 188, 190, or 192; and a light chain constant region comprising an amino acid sequence modified by 1-10 amino acid substitutions, 1-5 amino acid substitutions, or 1-3 amino acid substitutions compared to the amino acid sequence of SEQ ID NO: 194. In some embodiments, the antibody comprises a human IgG1 heavy chain constant region comprising amino acid sequence of any one of SEQ ID Nos: 174, 176, 178, 180, 182, 184, 186, 188, 190, or 192. In some embodiments, the antibody comprises a light chain constant region comprising the amino acid sequence of SEQ ID NO: 194. In some embodiments, the antibody comprises both a human IgG1 heavy chain constant region comprising the amino acid sequence of any one of SEQ ID Nos: 174, 176, 178, 180, 182, 184, 186, 188, 190, or 192; and a light chain constant region comprising the amino acid sequence of SEQ ID NO: 194.

In some cases, the antibody comprises a heavy chain (HC) comprising the amino acid sequence of SEQ ID NO: 86 followed by the amino acid sequence of SEQ ID NO: 178 and a light chain (LC) comprising the amino acid sequence of SEQ ID NO: 88 followed by the amino acid sequence of SEQ ID NO: 194. In some cases, the antibody comprises a heavy chain (HC) comprising the amino acid sequence of SEQ ID NO: 86 followed by the amino acid sequence of SEQ ID NO: 180 and a light chain (LC) comprising the amino acid sequence of SEQ ID NO: 88 followed by the amino acid sequence of SEQ ID NO: 194.

In some cases the antibody binds to an epitope on PAD4 comprising SEQ ID NO: 219 and SEQ ID NO: 220.

In some embodiments, the antibody specifically binds to PAD4 in an SPR assay with a K_{D} from 0.05 nM to 1 nM, from 0.1 nM to 1 nM, from 0.1 nM to 0.5 nM, from 0.05 nM to 0.5 nM, or from 0.05 nM to 0.1 nM, or of 0.09 nM, 0.1 nM, or 0.2 nM, for example, both in the presence of 1-2 mM calcium chloride (e.g., 1 mM calcium chloride) and in the absence of calcium ion (due to absence of added calcium salt as well as the presence of EDTA, such as 1 mM or 2 mM EDTA). See Tables 2 and 3. In some embodiments, the antibody binds specifically to human PAD4 but not to murine PAD4 as determined using SPR. In some embodiments, the antibody binds specifically to human PAD4 but not to cynomolgous monkey PAD4 as determined using SPR.

In some embodiments, the antibody has an ECM score of less than 20, less than 10, 4-10, 4-8, or 5-7. (See, for example Table 5.)

In some embodiments, the antibody has an immunogenicity score of 0-5%, 0-3%, or 1-2%, as measured using an Epivax® immunogenicity test. (See Table 10.) In some cases, the antibody is less immunogenic that one, two, or all three of Campath® (alemtuzumab), Rituxan® (rituximab), and Zenapax® (daclizumab), as measured in an in silico immunogenicity assay.

In some embodiments, the antibody inhibits PAD4 conversion of arginine in peptide substrate (TSTGGRQGSHH; SEQ ID NO: 216) to citrulline in vitro with an IC50 of 50-100 nM or 50-80 nM or 60-80 nM, for example, in an assay as shown in Example 4 below.

In some embodiments, the antibody inhibits the enzymatic activity of PAD4 in vitro in the presence of an antibody that binds and activates human PAD4 (an activating antibody). In some embodiments, the activating antibody is an antibody derived from a patient with rheumatoid arthritis. In some embodiments, the activating antibody is an antibody that is cross reactive for human PAD3 and human PAD4.

In some cases, the antibody reduces the amount of extracellular citrullinated histone H3 in LPS-stimulated human blood monocytes compared to an isotype control antibody. In some embodiments, the LPS-stimulated human blood monocytes are CD14+CD16-monocytes isolated from fresh human PBMCs. (See Example 17 herein.) In some embodiments, the antibody is capable of being internalized by LPS-stimulated human blood monocytes, such as by LPS-stimulated CD14+ human monocytes. (See Example 18.)

In some embodiments, the antibody inhibits PAD4 function in an inflamed lung. For example, this may be demonstrated by decreased citrullination of histone H3 or ITIH4 in broncheoalveolar lavage fluid (BALF) collected from the lung, as described in Example 25 herein. For example, BALF may be collected from mice with acute or chronic lung inflammation and assayed for the amount of citrullinated H3 or ITIH4 protein in the presence of the antibody and in the presence of a wild-type control. In some embodiments, the mice are human PAD4 knock-in mice. In some cases, the amount of citrullinated H3 is reduced by at least 10% or at least 15% and/or the amount of citrullinated ITIH4 is reduced by at least 50%, at least 70%, or at least 80% in the presence of the antibody in human PAD4 knock-in mice, compared to an isotope control (IC) antibody, for example, as calculated by the following formula: % inhibition=[(IC−naïve))−(PAD4 mAb−naïve)/(IC−naïve)]×100, in which the amount of citrullinated H3 or ITIH4 is measured for untreated (naïve) mice, mice receiving IC, and mice receiving an anti-PAD4 antibody.

In some embodiments, the antibody inhibits PAD4 function in an inflamed joint, as described in Example 26. This may be demonstrated, for example, by decreased citrullination of ITIH4 and/or PRG4 in joint tissue in a mouse model with acute or chronic joint inflammation, such as induced by LPS injection of a joint. In some cases, the mice may be human PAD4 knock-in mice. In some cases, the antibody reduces the amount of extracellular citrullinated PRG4 and/or citrullinated ITIH4 in mouse patella in an LPS-induced acute joint injury model with human PAD4 knock-in mice compared to an isotype control antibody. In some cases, the EC50 of reduction of citrullinated ITIH4 and/or citrullinated PRG4 is 2 nM or lower, 1 nM or lower, or 0.5-1.5 nM.

E. Antibody hz20-7

In some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of SEQ ID NO: 72, an HCDR2 comprising the amino acid sequence of SEQ ID NO: 73, and an HCDR3 comprising the amino acid sequence of SEQ ID NO: 74; and a light chain variable region (VL) comprising a light chain complementarity determining region 1 (LCDR1) comprising the amino acid sequence of SEQ ID NO: 75, an LCDR2 comprising the amino acid sequence of SEQ ID NO: 76, and an LCDR3 comprising the amino acid sequence of SEQ ID NO: 77. In some embodiments, the antibody comprises a VH that is at least 90%, at least 95%, at least 97%, or at least 99% identical to the amino acid sequence of SEQ ID NO: 106. In some embodiments, the antibody comprises a VL that is at least 90%, at least 95%, at least 97%, or at least 99% identical to the amino acid sequence of SEQ ID NO: 108. In some embodiments, the antibody comprises both a VH that is at least 90%, at least 95%, at least 97%, or at least 99% identical to the amino acid sequence of SEQ ID NO: 106; and a VL that is at least 90%, at least 95%, at least 97%, or at least 99% identical to the amino acid sequence of SEQ ID NO: 108. In some embodiments, the VH is at least 90% identical to the amino acid sequence of SEQ ID NO: 106 and the VL is at least 90% identical to the amino acid sequence of SEQ ID NO: 108. In some embodiments, the VH is at least 95% identical to the amino acid sequence of SEQ ID NO: 106 and the VL is at least 95% identical to the amino acid sequence of SEQ ID NO: 108. In some embodiments, the VH is at least 97% identical to the amino acid sequence of SEQ ID NO: 106 and the VL is at least 97% identical to the amino acid sequence of SEQ ID NO: 108. In some embodiments, the antibody comprises a VH comprising the amino acid sequence of SEQ ID No: 106 modified by 1-10 amino acid substitutions, 1-5 amino acid substitutions, or 1-3 amino acid substitutions. In some embodiments, the antibody comprises a VL comprising the amino acid sequence of SEQ ID No: 108 modified by 1-10 amino acid substitutions, 1-5 amino acid substitutions, or 1-3 amino acid substitutions. In some embodiments, the antibody comprises both a VH comprising the amino acid sequence of SEQ ID No: 106 modified by 1-10 amino acid substitutions, 1-5 amino acid substitutions, or 1-3 amino acid substitutions and a VL comprising the amino acid sequence of SEQ ID No: 108 modified by 1-10 amino acid substitutions, 1-5 amino acid substitutions, or 1-3 amino acid substitutions.

In some embodiments, the antibody comprises a VH comprising the amino acid sequence of SEQ ID No: 106. In some embodiments, the antibody comprises a VH comprising the amino acid sequence of SEQ ID No: 106 and a VL that is at least 90%, at least 95%, at least 97%, or at least 99% identical to the amino acid sequence of SEQ ID NO: 108. In some embodiments, the antibody comprises a VL comprising the amino acid sequence of SEQ ID No: 108. In some embodiments, the antibody comprises a VH that is at least 90%, at least 95%, at least 97%, or at least 99% identical to the amino acid sequence of SEQ ID NO: 106; and a VL comprising the amino acid sequence of SEQ ID No: 106. In some embodiments, the antibody comprises a VH comprising the amino acid sequence of SEQ ID No: 106 and a VL comprising the amino acid sequence of SEQ ID No: 108.

In some cases, the antibody is an IgG antibody, such as a human IgG1, IgG2, IgG3, or IgG4 antibody. In some embodiments, the antibody comprises a human IgG1 heavy chain constant region comprising an amino acid sequence at least 90%, at least 95%, at least 97%, or at least 99% identical to the amino acid sequence of any one of SEQ ID Nos: 174, 176, 178, 180, 182, 184, 186, 188, 190, or 192. In some embodiments, the antibody comprises a light chain constant region comprising an amino acid sequence at least 90%, at least 95%, at least 97%, or at least 99% identical to the amino acid sequence of SEQ ID NO: 194. In some embodiments, the antibody comprises both a human IgG1 heavy chain constant region comprising an amino acid sequence at least 90%, at least 95%, at least 97%, or at least 99% identical to the amino acid sequence of any one of SEQ ID Nos: 174, 176, 178, 180, 182, 184, 186, 188, 190, or 192; and a light chain constant region comprising an amino acid sequence at least 90%, at least 95%, at least 97%, or at least 99% identical to the amino acid sequence of SEQ ID NO: 194. In some cases, the antibody comprises a heavy chain constant region comprising an amino acid sequence modified by 1-10 amino acid substitutions, 1-5 amino acid substitutions, or 1-3 amino acid substitutions compared to the amino acid sequence of any one of SEQ ID Nos: 174, 176, 178, 180, 182, 184, 186, 188, 190, or 192. In some cases, the antibody comprises a light chain constant region comprising an amino acid sequence modified by 1-10 amino acid substitutions, 1-5 amino acid substitutions, or 1-3 amino acid substitutions compared to the amino acid sequence of SEQ ID NO: 194. In some cases, the antibody comprises both a heavy chain constant region comprising an amino acid sequence modified by 1-10 amino acid substitutions, 1-5 amino acid substitutions, or 1-3 amino acid substitutions compared to the amino acid sequence of any one of SEQ ID Nos: 174, 176, 178, 180, 182, 184, 186, 188, 190, or 192; and a light chain constant region comprising an amino acid sequence modified by 1-10 amino acid substitutions, 1-5 amino acid substitutions, or 1-3 amino acid substitutions compared to the amino acid sequence of SEQ ID NO: 194. In some embodiments, the antibody comprises a human IgG1 heavy chain constant region comprising amino acid sequence of any one of SEQ ID Nos: 174, 176, 178, 180, 182, 184, 186, 188, 190, or 192. In some embodiments, the antibody comprises a light chain constant region comprising the amino acid sequence of SEQ ID NO: 194. In some embodiments, the antibody comprises both a human IgG1 heavy chain constant region comprising the amino acid sequence of any one of SEQ ID Nos: 174, 176, 178, 180, 182, 184, 186, 188, 190, or 192; and a light chain constant region comprising the amino acid sequence of SEQ ID NO: 194.

In some cases, the antibody comprises a heavy chain (HC) comprising the amino acid sequence of SEQ ID NO: 106 followed by the amino acid sequence of SEQ ID NO: 178 and a light chain (LC) comprising the amino acid sequence of SEQ ID NO: 108 followed by the amino acid sequence of SEQ ID NO: 194. In some cases, the antibody comprises a heavy chain (HC) comprising the amino acid sequence of SEQ ID NO: 106 followed by the amino acid sequence of SEQ ID NO: 180 and a light chain (LC) comprising the amino acid sequence of SEQ ID NO: 108 followed by the amino acid sequence of SEQ ID NO: 194.

In some cases the antibody binds to an epitope on PAD4 comprising SEQ ID NO: 219 and SEQ ID NO: 220.

In some embodiments, the antibody specifically binds to PAD4 in an SPR assay with a K_{D} from 0.1 nM to 1 nM, or from 0.3 nM to 1 nM, for example, both in the presence of 1-2 mM calcium chloride (e.g., 1 mM calcium chloride) and in the absence of calcium ion (due to absence of added calcium salt as well as the presence of EDTA, such as 1 mM or 2 mM EDTA). See Tables 2 and 3. In some embodiments, the antibody binds specifically to human PAD4 but not to murine PAD4 as determined using SPR. In some embodiments, the antibody binds specifically to human PAD4 but not to cynomolgous monkey PAD4 as determined using SPR.

In some embodiments, the antibody has an ECM score of less than 20, less than 10, 2-10, 4-8, or 4-7. (See, for example Table 5.)

In some embodiments, the antibody has an immunogenicity score of 0-5%, 0-3%, or 0-2%, as measured using an Epivax® immunogenicity test. In some cases, the antibody has a score of 0.5-1%. (See Table 10.) In some cases, the antibody is less immunogenic that one, two, or all three of Campath® (alemtuzumab), Rituxan® (rituximab), and Zenapax® (daclizumab), as measured in an in silico immunogenicity assay.

In some embodiments, the antibody inhibits PAD4 conversion of arginine in peptide substrate (TSTGGRQGSHH; SEQ ID NO: 216) to citrulline in vitro with an IC50 of 50-100 nM or 50-80 nM or 60-80 nM, for example, in an assay as shown in Example 4 below.

In some embodiments, the antibody inhibits the enzymatic activity of PAD4 in vitro in the presence of an antibody that binds and activates human PAD4 (an activating antibody). In some embodiments, the activating antibody is an antibody derived from a patient with rheumatoid arthritis. In some embodiments, the activating antibody is an antibody that is cross reactive for human PAD3 and human PAD4.

In some cases, the antibody reduces the amount of extracellular citrullinated histone H3 in LPS-stimulated human blood monocytes compared to an isotype control antibody. In some embodiments, the LPS-stimulated human blood monocytes are CD14+CD16− monocytes isolated from fresh human PBMCs. (See Example 17 herein.) In some embodiments, the antibody is capable of being internalized by LPS-stimulated human blood monocytes, such as by LPS-stimulated CD14+ human monocytes. (See Example 18.)

In some embodiments, the antibody inhibits PAD4 function in an inflamed lung. For example, this may be demonstrated by decreased citrullination of histone H3 or ITIH4 in broncheoalveolar lavage fluid (BALF) collected from the lung, as described in Example 25 herein. For example, BALF may be collected from mice with acute or chronic lung inflammation and assayed for the amount of citrullinated H3 or ITIH4 protein in the presence of the antibody and in the presence of a wild-type control. In some embodiments, the mice are human PAD4 knock-in mice. In some cases, the amount of citrullinated H3 is reduced by at least 10% or at least 15% and/or the amount of citrullinated ITIH4 is reduced by at least 50%, at least 70%, or at least 80% in the presence of the antibody in human PAD4 knock-in mice, compared to an isotope control (IC) antibody, for example, as calculated by the following formula: % inhibition=[(IC−naïve))−(PAD4 mAb−naîve)/(IC−naîve)]×100, in which the amount of citrullinated H3 or ITIH4 is measured for untreated (naïve) mice, mice receiving IC, and mice receiving an anti-PAD4 antibody.

F. pH Dependent Antibody hz13-5 VH_D31H:Vk_I30H

In some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of positions 26-35 of SEQ ID NO: 168, an HCDR2 comprising the amino acid sequence of positions 50-66 of SEQ ID NO: 168, and an HCDR3 comprising the amino acid sequence of positions 99-108 of SEQ ID NO: 168; and a light chain variable region (VL) comprising a light chain complementarity determining region 1 (LCDR1) comprising the amino acid sequence residues 24-38 of SEQ ID NO: 172, an LCDR2 comprising the amino acid sequence of residues 54-60 of SEQ ID NO: 172, and an LCDR3 comprising the amino acid sequence of residues 93-101 of SEQ ID NO: 172. In some embodiments, the antibody comprises a VH that is at least 90%, at least 95%, at least 97%, or at least 99% identical to the amino acid sequence of SEQ ID NO: 168. In some embodiments, the antibody comprises a VL that is at least 90%, at least 95%, at least 97%, or at least 99% identical to the amino acid sequence of SEQ ID NO: 172. In some embodiments, the antibody comprises both a VH that is at least 90%, at least 95%, at least 97%, or at least 99% identical to the amino acid sequence of SEQ ID NO: 168; and a VL that is at least 90%, at least 95%, at least 97%, or at least 99% identical to the amino acid sequence of SEQ ID NO: 172. In some embodiments, the antibody comprises both a VH that is at least 90% identical to the amino acid sequence of SEQ ID NO: 168; and a VL that is at least 90% identical to the amino acid sequence of SEQ ID NO: 172. In some embodiments, the antibody comprises both a VH that is at least 95% identical to the amino acid sequence of SEQ ID NO: 168; and a VL that is at least 95% identical to the amino acid sequence of SEQ ID NO: 172. In some embodiments, the antibody comprises both a VH that is at least 97% identical to the amino acid sequence of SEQ ID NO: 168; and a VL that is at least 97% identical to the amino acid sequence of SEQ ID NO: 172. In some embodiments, the antibody comprises a VH comprising the amino acid sequence of SEQ ID No: 168 modified by 1-10 amino acid substitutions, 1-5 amino acid substitutions, or 1-3 amino acid substitutions. In some embodiments, the antibody comprises a VL comprising the amino acid sequence of SEQ ID No: 172 modified by 1-10 amino acid substitutions, 1-5 amino acid substitutions, or 1-3 amino acid substitutions. In some embodiments, the antibody comprises both a VH comprising the amino acid sequence of SEQ ID No: 168 modified by 1-10 amino acid substitutions, 1-5 amino acid substitutions, or 1-3 amino acid substitutions and a VL comprising the amino acid sequence of SEQ ID No: 172 modified by 1-10 amino acid substitutions, 1-5 amino acid substitutions, or 1-3 amino acid substitutions. In some embodiments, the VH of the antibody comprises a glycine at Kabat position 94 (position 98 of SEQ ID NO: 10).

In some embodiments, the antibody comprises a VH comprising the amino acid sequence of SEQ ID No: 58. In some embodiments, the antibody comprises a VH comprising the amino acid sequence of SEQ ID No: 168; and a VL that is at least 90%, at least 95%, at least 97%, or at least 99% identical to the amino acid sequence of SEQ ID NO: 172. In some embodiments, the antibody comprises a VL comprising the amino acid sequence of SEQ ID No: 172. In some embodiments, the antibody comprises a VH that is at least 90%, at least 95%, at least 97%, or at least 99% identical to the amino acid sequence of SEQ ID NO: 168; and a VL comprising the amino acid sequence of SEQ ID No: 72. In some embodiments, the antibody comprises a VH comprising the amino acid sequence of SEQ ID No: 168 and a VL comprising the amino acid sequence of SEQ ID No: 172.

In some cases, the antibody comprises a heavy chain (HC) comprising the amino acid sequence of SEQ ID NO: 168 followed by the amino acid sequence of SEQ ID NO: 178 and a light chain (LC) comprising the amino acid sequence of SEQ ID NO: 172 followed by the amino acid sequence of SEQ ID NO: 194. In some cases, the antibody comprises a heavy chain (HC) comprising the amino acid sequence of SEQ ID NO: 168 followed by the amino acid sequence of SEQ ID NO: 180 and a light chain (LC) comprising the amino acid sequence of SEQ ID NO: 172 followed by the amino acid sequence of SEQ ID NO: 194.

In some cases, the antibody is an IgG antibody, such as a human IgG1, IgG2, IgG3, or IgG4 antibody. In some embodiments, the antibody comprises a human IgG1 heavy chain constant region comprising an amino acid sequence at least 90%, at least 95%, at least 97%, or at least 99% identical to the amino acid sequence of any one of SEQ ID Nos: 174, 176, 178, 180, 182, 184, 186, 188, 190, or 192. In some embodiments, the antibody comprises a light chain constant region comprising an amino acid sequence at least 90%, at least 95%, at least 97%, or at least 99% identical to the amino acid sequence of SEQ ID NO: 194. In some embodiments, the antibody comprises both a human IgG1 heavy chain constant region comprising an amino acid sequence at least 90%, at least 95%, at least 97%, or at least 99% identical to the amino acid sequence of any one of SEQ ID Nos: 174, 176, 178, 180, 182, 184, 186, 188, 190, or 192; and a light chain constant region comprising an amino acid sequence at least 90%, at least 95%, at least 97%, or at least 99% identical to the amino acid sequence of SEQ ID NO: 194. In some cases, the antibody comprises a heavy chain constant region comprising an amino acid sequence modified by 1-10 amino acid substitutions, 1-5 amino acid substitutions, or 1-3 amino acid substitutions compared to the amino acid sequence of any one of SEQ ID Nos: 174, 176, 178, 180, 182, 184, 186, 188, 190, or 192. In some cases, the antibody comprises a light chain constant region comprising an amino acid sequence modified by 1-10 amino acid substitutions, 1-5 amino acid substitutions, or 1-3 amino acid substitutions compared to the amino acid sequence of SEQ ID NO: 194. In some cases, the antibody comprises both a heavy chain constant region comprising an amino acid sequence modified by 1-10 amino acid substitutions, 1-5 amino acid substitutions, or 1-3 amino acid substitutions compared to the amino acid sequence of any one of SEQ ID Nos: 174, 176, 178, 180, 182, 184, 186, 188, 190, or 192; and a light chain constant region comprising an amino acid sequence modified by 1-10 amino acid substitutions, 1-5 amino acid substitutions, or 1-3 amino acid substitutions compared to the amino acid sequence of SEQ ID NO: 194. In some embodiments, the antibody comprises a human IgG1 heavy chain constant region comprising amino acid sequence of any one of SEQ ID Nos: 174, 176, 178, 180, 182, 184, 186, 188, 190, or 192. In some embodiments, the antibody comprises a light chain constant region comprising the amino acid sequence of SEQ ID NO: 194. In some embodiments, the antibody comprises both a human IgG1 heavy chain constant region comprising the amino acid sequence of any one of SEQ ID Nos: 174, 176, 178, 180, 182, 184, 186, 188, 190, or 192; and a light chain constant region comprising the amino acid sequence of SEQ ID NO: 194.

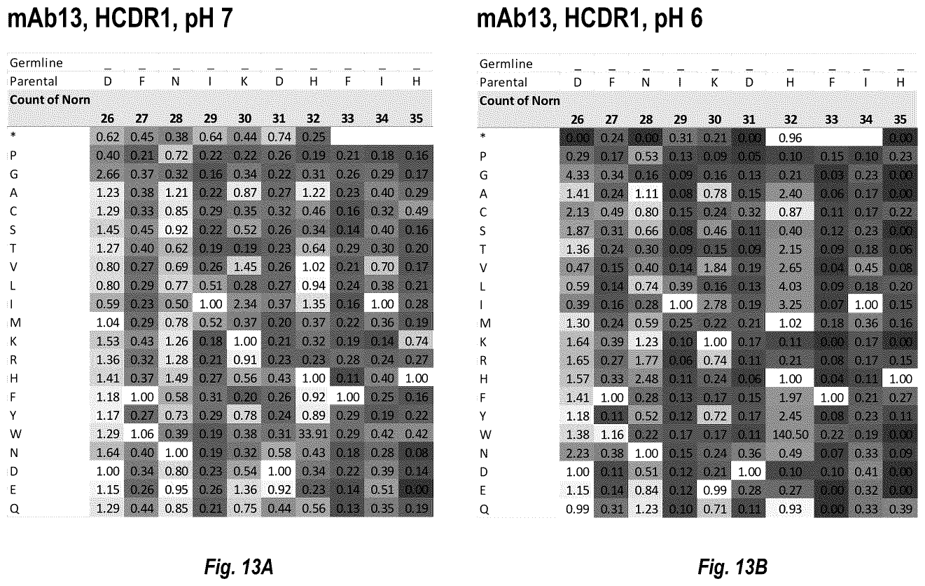

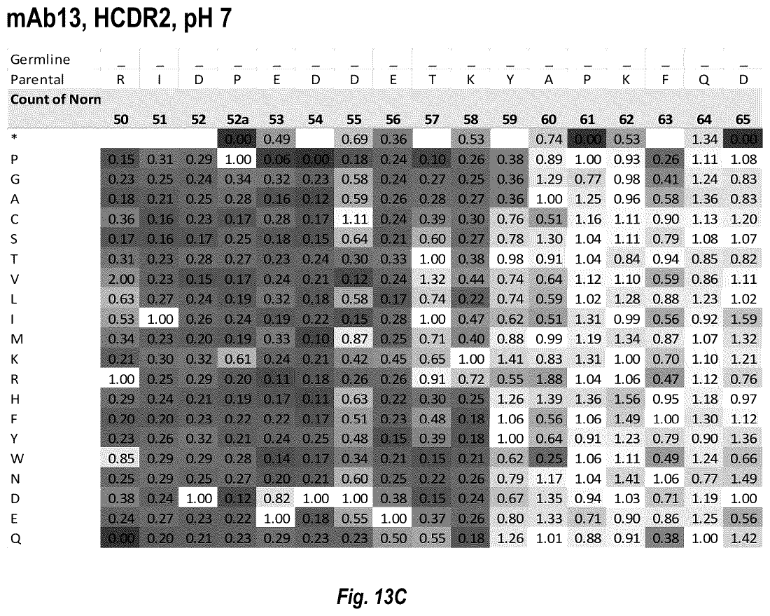

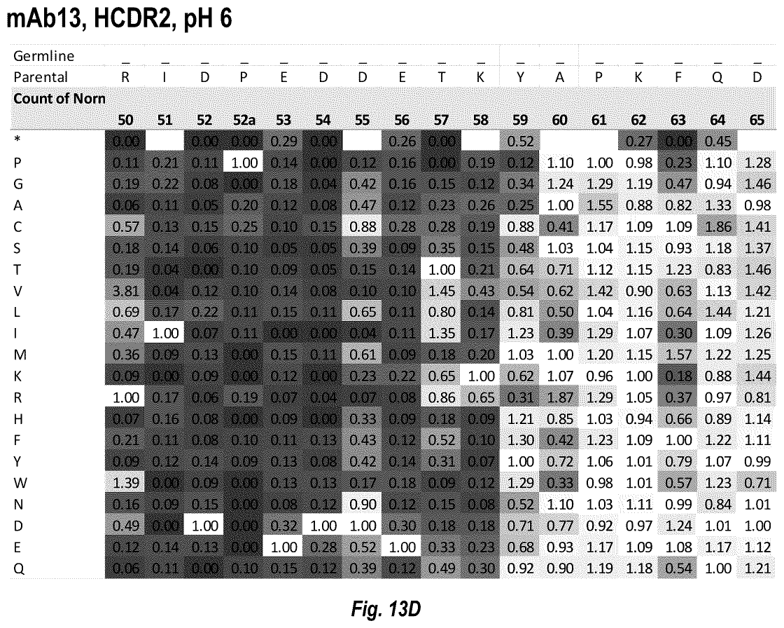

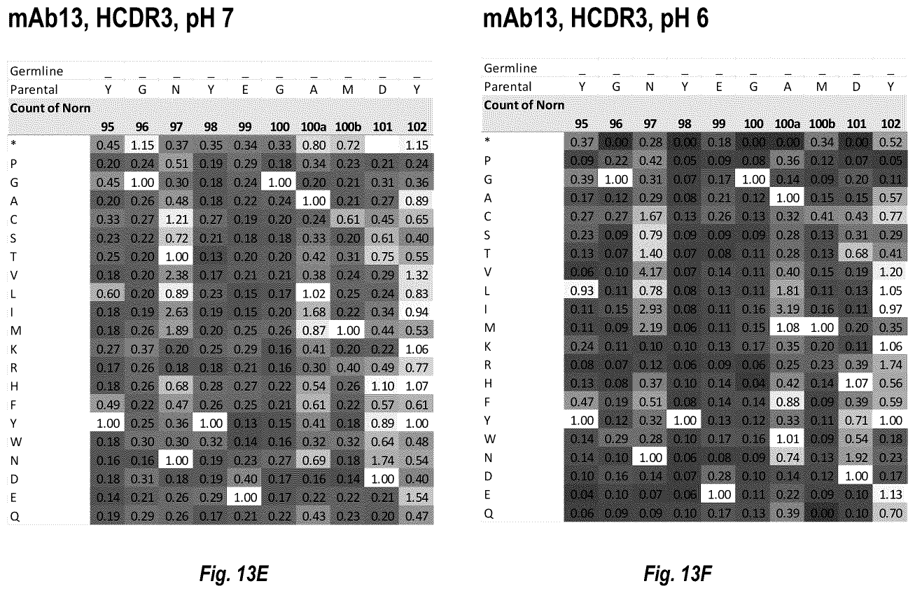

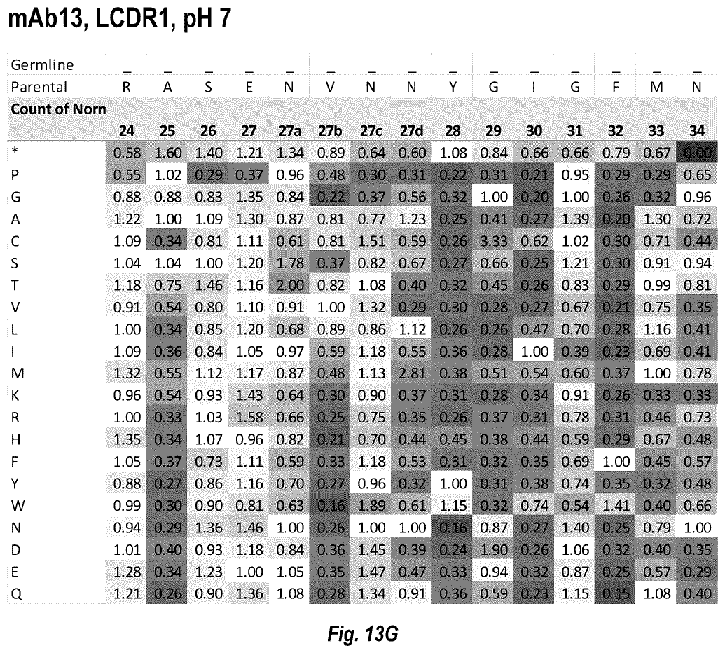

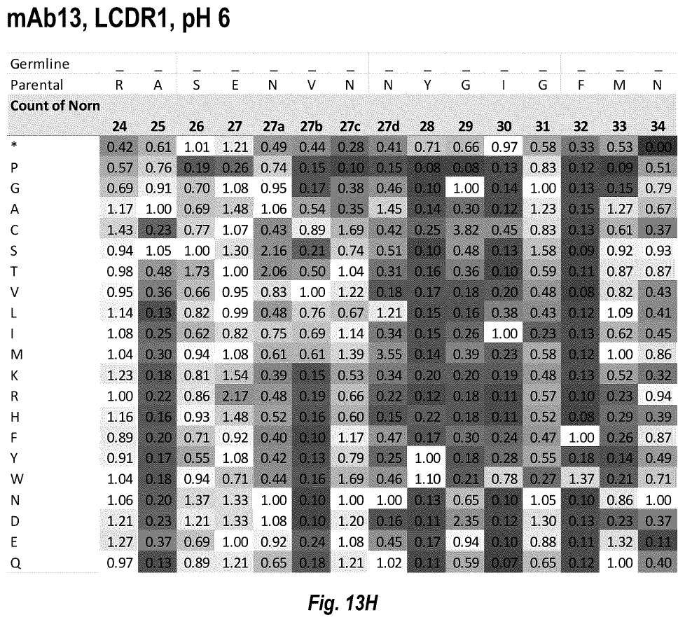

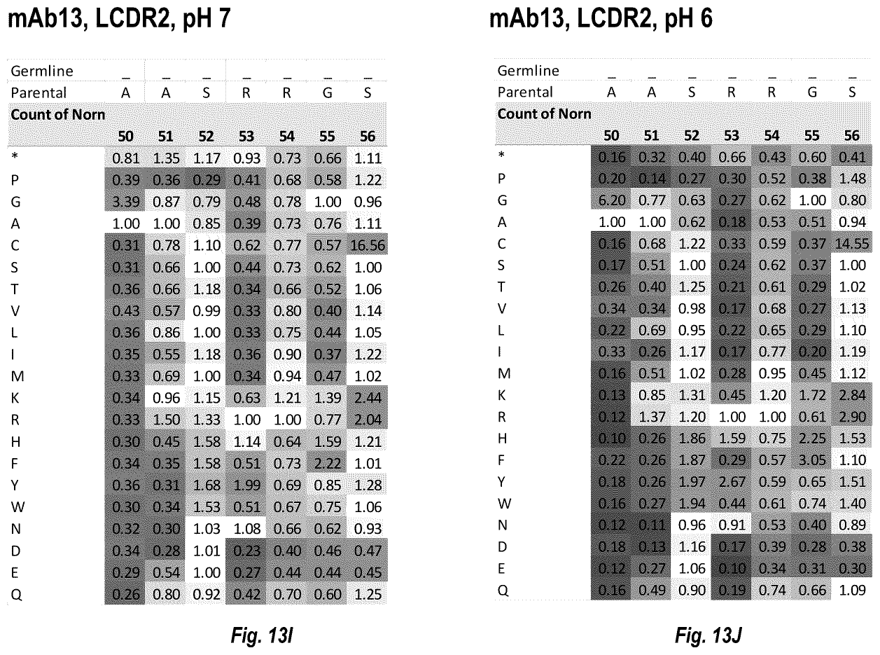

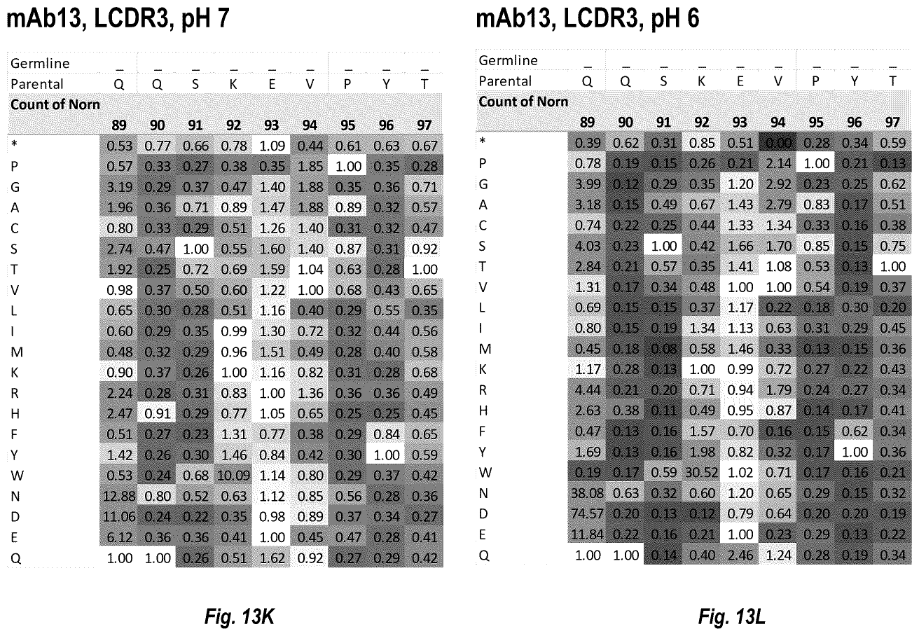

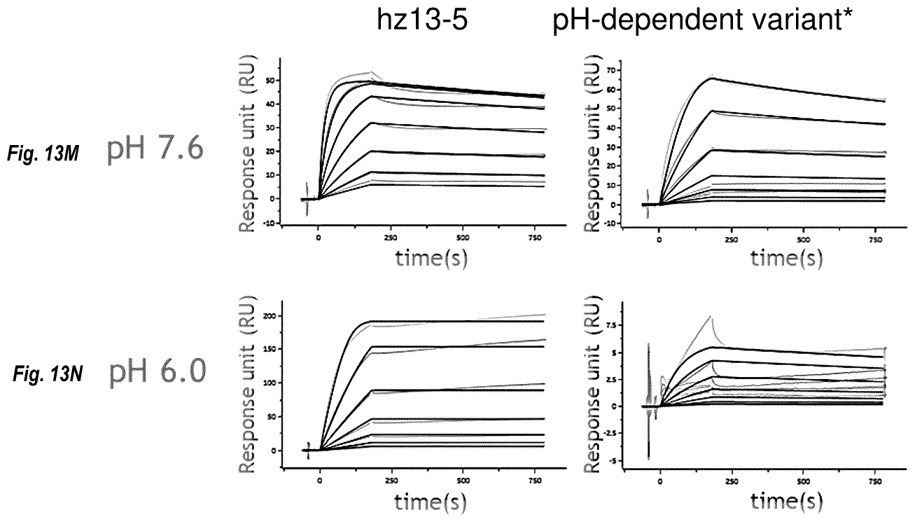

In the case of antibody hz13-5 VH_D31H:Vk_I30H, pH dependent binding to human PAD4 was observed. The binding activity of this mutant at pH 7.6 was comparable to (unmutated) hz13-5 binding to human PAD4 (see FIG. 13M). Whereas the parent hz13-5 retained binding at pH 6.0, the mutant hz13-5 VH_D31H::Vk_I30H demonstrated a loss of binding to human PAD4 at pH 6.0. See FIG. 13N.

G. Murine Surrogate Antibody mumAb

In some embodiments, the disclosure relates to an isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of positions 31-35 of SEQ ID NO: 208, an HCDR2 comprising the amino acid sequence of positions 50-66 of SEQ ID NO: 208, and an HCDR3 comprising the amino acid sequence of positions 99-107 of SEQ ID NO: 208; and a light chain variable region (VL) comprising a light chain complementarity determining region 1 (LCDR1) comprising the amino acid sequence of residues 24-38 of SEQ ID NO: 210, an LCDR2 comprising the amino acid sequence of residues 54-60 of SEQ ID NO: 210, and an LCDR3 comprising the amino acid sequence of residues 93-101 of SEQ ID NO: 210. In some embodiments, the antibody comprises a VH that is at least 90%, at least 95%, at least 97%, or at least 99% identical to the amino acid sequence of SEQ ID NO: 208. In some embodiments, the antibody comprises a VL that is at least 90%, at least 95%, at least 97%, or at least 99% identical to the amino acid sequence of SEQ ID NO: 210. In some embodiments, the antibody comprises both a VH that is at least 90%, at least 95%, at least 97%, or at least 99% identical to the amino acid sequence of SEQ ID NO: 208; and a VL that is at least 90%, at least 95%, at least 97%, or at least 99% identical to the amino acid sequence of SEQ ID NO: 210. In some embodiments, the antibody comprises both a VH that is at least 90% identical to the amino acid sequence of SEQ ID NO: 208; and a VL that is at least 90% identical to the amino acid sequence of SEQ ID NO: 210. In some embodiments, the antibody comprises both a VH that is at least 95% identical to the amino acid sequence of SEQ ID NO: 208; and a VL that is at least 95% identical to the amino acid sequence of SEQ ID NO: 210. In some embodiments, the antibody comprises both a VH that is at least 97% identical to the amino acid sequence of SEQ ID NO: 208; and a VL that is at least 97% identical to the amino acid sequence of SEQ ID NO: 210. In some embodiments, the antibody comprises a VH comprising the amino acid sequence of SEQ ID No: 208 modified by 1-10 amino acid substitutions, 1-5 amino acid substitutions, or 1-3 amino acid substitutions. In some embodiments, the antibody comprises a VL comprising the amino acid sequence of SEQ ID No: 210 modified by 1-10 amino acid substitutions, 1-5 amino acid substitutions, or 1-3 amino acid substitutions. In some embodiments, the antibody comprises both a VH comprising the amino acid sequence of SEQ ID No: 208 modified by 1-10 amino acid substitutions, 1-5 amino acid substitutions, or 1-3 amino acid substitutions and a VL comprising the amino acid sequence of SEQ ID No: 210 modified by 1-10 amino acid substitutions, 1-5 amino acid substitutions, or 1-3 amino acid substitutions.

In some embodiments, the antibody comprises a VH comprising the amino acid sequence of SEQ ID No: 208. In some embodiments, the antibody comprises a VH comprising the amino acid sequence of SEQ ID No: 208; and a VL that is at least 90%, at least 95%, at least 97%, or at least 99% identical to the amino acid sequence of SEQ ID NO: 210. In some embodiments, the antibody comprises a VL comprising the amino acid sequence of SEQ ID No: 210. In some embodiments, the antibody comprises a VH that is at least 90%, at least 95%, at least 97%, or at least 99% identical to the amino acid sequence of SEQ ID NO: 208; and a VL comprising the amino acid sequence of SEQ ID No: 210. In some embodiments, the antibody comprises a VH comprising the amino acid sequence of SEQ ID No: 208 and a VL comprising the amino acid sequence of SEQ ID No: 210.

In some cases, the antibody comprises a heavy chain (HC) comprising the amino acid sequence of SEQ ID NO: 212 and a light chain (LC) comprising the amino acid sequence of SEQ ID NO: 214.

The disclosure also encompasses, for example, one or more nucleic acid molecules encoding an anti-murine PAD4 antibody as described above, a vector comprising one or more nucleic acid molecules encoding the anti-murine PAD4 antibody, and a host cell or animal model that expresses the anti-murine PAD4 antibody (i.e., a host cell or animal model comprising a nucleic acid or vector encoding the anti-murine PAD4 antibody). Such a nucleic acid molecule, vector, or host cell may be as described in the sections that follow herein. Exemplary vectors include DNA vectors, RNA vectors (e.g., mRNA and circular RNA, self-amplifying RNA vectors, etc.), phage vectors, viral vectors (e.g., pox virus vectors, vaccinia virus vectors, adenovirus vectors, modified vaccinia virus Ankara (MVA) vectors, etc.), retroviral vectors, etc. Exemplary animal models include, for instance, murine models in which mice are administered with either an anti-murine PAD4 antibody as described above, or one or more nucleic acid molecules, vectors, or host cells that encode such an anti-murine PAD4 antibody.

Murine surrogate antibody mumAb was observed to have several in vitro and in vivo properties similar to those of other antibodies described herein, such as humanized clone 13 or clone 20 derivatives. For example, in some cases, the antibody inhibits PAD4 function in an inflamed lung. For example, this may be demonstrated by decreased citrullination of histone H3 or ITIH4 in broncheoalveolar lavage fluid (BALF) collected from the lung, as described in Example 20 herein. For example, BALF may be collected from mice with a normal murine PAD4 with acute or chronic lung inflammation and assayed for the amount of citrullinated H3 or ITIH4 protein in the presence of the antibody and in the presence of a wild-type control. In some cases, the amount of citrullinated H3 is reduced by at least 15%, at least 30%, at least 40%, at least 50%, or at least 60%, and/or the amount of citrullinated ITIH4 is reduced by at least 50%, at least 70%, or at least 80% in the presence of the antibody in the mice, compared to an isotope control (IC) antibody, for example, as calculated by the following formula: % inhibition=[(IC−naïve))−(PAD4 mAb−naïve)/(IC−naïve)]×100, in which the amount of citrullinated H3 or ITIH4 is measured for untreated (naïve) mice, mice receiving IC, and mice receiving an anti-PAD4 antibody.

In some embodiments, the antibody inhibits PAD4 function in an inflamed joint, for instance, as exemplified and described in Examples 21 and 22. This may be demonstrated, for example, by decreased citrullination of ITIH4 and/or PRG4 in joint tissue in a mouse model with normal murine PAD4 with acute or chronic joint inflammation, such as induced by LPS injection of a joint. In some cases, the antibody reduces the amount of extracellular citrullinated PRG4 and/or citrullinated ITIH4 in mouse patella in an LPS-induced acute joint injury model with human PAD4 knock-in mice compared to an isotype control antibody.

In some embodiments, an antibody herein may reduce pristane-induced extracellular trap forming neutrophils (NETosis) and/or extracellular trap forming monocytes (METosis). (See Example 23.) For example, mice may be injected intraperitoneally with pristane, for example, following treatment with an antibody herein or with an isotype control antibody. In some cases, in such a pristane-induced mouse model, the antibody reduces citrullination of H3 in neutrophils, monocytes, M1 macrophages, and/or M2 macrophages in peritoneal fluid compared to an isotype control antibody. In further cases, the antibody reduces the amount of soluble markers of neutrophils and monocytes/macrophages in the mice in peritoneal fluid, such as elastase, MPO, MIP-2 alpha, GRO alpha/KC, MCP1, MIP 1beta, IL6, and MIP3 alpha. (See Example 23.)

In some embodiments, in a collagen-induced arthritis mouse model, the antibody inhibits PAD4-dependent responses. (See Example 24.) For example, in some embodiments, an antibody herein significantly reduces the arthritis clinical score of mice in the arthritis model compared to an isotype control, according to the following scale: (1) normal; (2) mild, with definite redness and swelling of the ankle or wrist, or with apparent redness and swelling limited to individual digits, regardless of the number of affected digits; (3) moderate redness and swelling of ankle or wrist; (4) severe redness and swelling of the entire paw including digits; (5) maximally inflamed limb with involvement of multiple joints. For example, in some cases, the antibody also reduces NETosis and METosis as assessed by SG+ MPO+ neutrophils, and SG+ MPO+ monocytes/macrophages, respectively, and/or reduces soluble markers of monocytes/macrophages, such as elastase and MPO, and or reduces the proportion of citrulline in H3 protein in neutrophils, monocytes, and/or macrophages. (See Example 24.)

### VI. Nucleic Acid Molecules Encoding Anti-PAD4 Antibodies

Nucleic acid molecules comprising polynucleotides that encode one or more chains of anti-PAD4 antibodies are provided. In some embodiments, a nucleic acid molecule comprises a polynucleotide that encodes a heavy chain or a light chain of an anti-PAD4 antibody. In some embodiments, a nucleic acid molecule comprises both a polynucleotide that encodes a heavy chain and a polynucleotide that encodes a light chain, of an anti-PAD4 antibody. In some embodiments, a first nucleic acid molecule comprises a first polynucleotide that encodes a heavy chain and a second nucleic acid molecule comprises a second polynucleotide that encodes a light chain.

Appropriate examples of polynucleotides for encoding heavy chain and light chain polypeptides, such as a VH, VL, HC, or LC of an antibody herein are provided in the sequence table below, or include variants of those sequences that are degenerate to the provided sequences, i.e., containing one or more codon swaps compared to the provided sequences. For example, given that the genetic code is redundant in that in many cases more than one codon can code for a single amino acid residue, one codon can be swapped for another codon that encodes the same amino acid residue in certain situations, such as due to codon preferences by particular host cells used to express the antibodies.

In some such embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain and a light chain linked together.

In some embodiments, a polynucleotide encoding a heavy chain or light chain of an anti-PAD4 antibody comprises a nucleotide sequence that encodes a leader sequence, which, when translated, is located at the N terminus of the heavy chain or light chain. The leader sequence may be the native heavy or light chain leader sequence, or may be another heterologous leader sequence.

Nucleic acid molecules may be constructed using recombinant DNA techniques conventional in the art. In some embodiments, a nucleic acid molecule is an expression vector that is suitable for expression in a selected host cell.

### VII. Anti-PAD4 Antibody Expression and Production

A. Vectors

Vectors comprising polynucleotides that encode anti-PAD4 heavy chains and/or anti-PAD4 light chains are provided. Vectors comprising polynucleotides that encode anti-PAD4 heavy chains and/or anti-PAD4 light chains are also provided. Such vectors include, but are not limited to, DNA vectors, RNA vectors (e.g., mRNA and circular RNA, self-amplifying RNA vectors, etc.), phage vectors, viral vectors (e.g., pox virus vectors, vaccinia virus vectors, adenovirus vectors, modified vaccinia virus Ankara (MVA) vectors, etc.), retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is an scFv.

In some embodiments, a first vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a vector is selected that is optimized for expression of polypeptides in CHO or CHO-derived cells, or in NSO cells. Exemplary such vectors are described, e.g., in Running Deer et al., Biotechnol. Prog. 20:880-889 (2004).

In some embodiments, a vector is chosen for in vivo expression anti-PAD4 heavy chains and/or anti-PAD4 light chains in animals, including humans. In some such embodiments, expression of the polypeptide is under the control of a promoter that functions in a tissue-specific manner. For example, liver-specific promoters are described, e.g., in PCT Publication No. WO 2006/076288.

B. Host Cells

In various embodiments, anti-PAD4 heavy chains and/or anti-PAD4 light chains may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S and DG44 cells; PER.C6® cells (Crucell); and NSO cells. In some embodiments, anti-PAD4 heavy chains and/or anti-PAD4 light chains may be expressed in yeast. See, e.g., U.S. Publication No. US 2006/0270045 A1. In some embodiments, a particular eukaryotic host cell is selected based on its ability to make desired post-translational modifications to the anti-PAD4 heavy chains and/or anti-PAD4 light chains. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, e.g., in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

In some embodiments, one or more polypeptides may be produced in vivo in an animal that has been engineered or transfected with one or more nucleic acid molecules encoding the polypeptides, according to any suitable method.

C. Purification of Anti-PAD4 Antibodies

Anti-PAD4 antibodies may be purified by any suitable method. Such methods include, but are not limited to, the use of affinity matrices or hydrophobic interaction chromatography or size exclusion chromatography. (See, for example, Example 2 for description of purification of humanized antibodies.)

D. Cell-Free Production of Anti-PAD4 Antibodies

In some embodiments, an anti-PAD4 antibody is produced in a cell-free system. Nonlimiting exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

### VIII. Therapeutic Compositions and Methods

A. Methods of Treating Diseases Using Anti-PAD4 Antibodies

Antibodies of the invention, and compositions comprising antibodies of the invention, are provided for use in methods of treatment of a disease or disorder in a subject, e.g., a human or other animal. Methods of treating disease comprising administering anti-PAD4 antibodies are also provided. The terms “disease” and “disorder” are used interchangeably herein in the context of an indication to be treated.

In some embodiments, the disorder is cancer or an autoimmune disorder or an infectious disease. In some embodiments, the disorder is a disorder associated with NETosis, METosis, presence of anti-citrullinated protein antibodies (ACPA), increased PAD4 expression, or increased PAD4 activity such as increased citrullination of polypeptides such as histone H3.

For example, citrullination by PAD4 is a stress response and may serve as a signal for removal of stressed cells. (Brentville et al., Oncoimmunology 8: e1576490 (2019).) Proteins citrullinated by PAD4 become antigenic substrates and are targets for both cellular (i.e., T cell) and humoral (i.e., B cell-derived antibody) adaptive immune responses. (See, e.g., Curran et al. Nat. Rev. Rheumatol. 16: 301-15 (2020); Brentville et al.) Thus, PAD4 activity may lead to generation of anti-citrullinated protein antibodies (ACPA). In neutrophils, PAD4 also plays a role in a process called NETosis, by which neutrophils extrude a complex of decondensed chromatin structures containing a DNA scaffold, citrullinated histones, and anti-bacterial neutrophilic granules. (Li et al. J. Exp. Med. 207: 1853-62 (2010).) These extruded complexes are called neutrophil extracellular traps (NET) and, during NETosis, these NETs trap and kill invading microbes as part of the innate immune response. (Chamardani et al., Mol. Cell. Biochem. 477: 673-88 (2022).) A similar process involving monocytes and macrophages is called METosis and involves formation of monocyte extracellular traps (MET).

Certain diseases and disorders are associated with NETosis, METosis, presence of anti-citrullinated protein antibodies (ACPA), increased PAD4 expression, or increased PAD4 activity such as increased citrullination of polypeptides such as histone H3. The disclosure herein also contemplates use of an antibody herein for treating such diseases and disorders. The disclosure herein also contemplates use of an antibody herein for inhibiting NETosis or METosis in a subject. The disclosure herein further contemplates use of an antibody herein for inhibiting citrullination in a subject. Inhibition of citrullination may comprise citrullination at one or more proteins found in serum, whole blood, blood plasma, blood supernatant, or synovial fluid or in other bodily fluids or tissues. Examples include, for instance, in one or more of proteoglycan 4 (PRG4), fibrinogen A (FGA), Inter-alpha-trypsin inhibitor heavy chain H4 (ITIH4), alpha-1-microglobulin/bikunin precursor (AMBP) and gelsolin (GSN). In some cases, the anti-PAD4 antibody may be used to inhibit NETosis or METosis in a subject with, for example, an autoimmune disease, or another condition disclosed herein. In some cases, the anti-PAD4 antibody may be used to inhibit citrullination in a subject with, for example, an autoimmune disease, or another condition disclosed herein.

In some cases, an antibody herein may be used to “prevent onset or recurrence” of a disorder, such as an autoimmune disorder, or a disorder associated with NETosis or METosis, presence of anti-citrullinated protein antibodies (ACPA), increased PAD4 expression, or increased PAD4 activity such as increased citrullination of polypeptides such as histone H3. As used herein, “preventing onset or recurrence” means that inhibiting the onset or recurrence of at least one symptom associated with the disorder in a subject, such as in a subject determined to be susceptible to developing symptoms or a subject in remission or whose previous symptoms have abated, for example due to treatment with other therapies (e.g., inhibiting onset of inflammation of a joint in an RA subject, or recurrence of joint inflammation in an RA subject). As used herein, “preventing onset or recurrence” also encompasses inhibiting an increase in at least one symptom of the disorder, such as in a subject whose symptoms have abated to a low level (e.g., a significant increase in joint inflammation in an RA subject). Thus, in such cases, an antibody herein may be provided to a subject who does not presently show symptoms of the disorder, in order to stop or slow the onset of symptoms, or the antibody may be provided to a subject who is in remission in order to stop or slow the onset of new symptoms of the disorder or the onset of related disorders. For example, a subject may have sub-clinical evidence of a disorder, such as one or more of presence of ACPA, presence of rheumatoid factor (RF), or increased PAD4 expression, or increased citrullination of polypeptides, for example, as detected in a biological sample such as whole blood, plasma, serum, blood supernatant, or synovial fluid, but may not yet show symptoms of the disorder.

In some embodiments, the disorder is an autoimmune disorder. For instance, a variety of data suggest that PAD4 plays a role in autoimmune diseases such as rheumatoid arthritis (RA), lupus (including systemic lupus erythematosus (SLE), lupus nephritis, vasculitis (including anti-neutrophilic cytoplasmic antibody (ANCA)-associated vasculitis, inflammatory bowel disease (IBD) (including ulcerative colitis and Crohn's disease), thrombosis (e.g., venous thrombosis), antiphospholipid antibody syndrome, and cystic fibrosis. (See, e.g., Curran et al.; Yadav et al., J. Cyst. Fibros. 18: 636-45 (2018); Wang et al., Front. Immunol. 13: 895216 (2022); Fresneda Alarcon et al. Frong. Immunol. 12: 649693 (2021); Weeding et al., Clin. Immunol. 196: 110-116 (2018); Xu et al., Chinese J. Microbiology and Immunology 12: 115-121 (2020); Yoshida et al., Clin. Kidney J. 6: 308-12 (2013); O'Sullivan et al., Rheumatology, 58(Suppl. 2): kez061.024 (2019); Pan et al., Authorea Preprints, 2021, DOI: 10.22541/au.161590650.07168461/v1.)

In some embodiments, the autoimmune disorder comprises or is rheumatoid arthritis (RA). In some embodiments, the disorder is RA, or the subject to be treated has been diagnosed with RA. In some embodiments, the subject is considered at risk of developing RA. In some embodiments, the RA is juvenile-onset RA, juvenile idiopathic arthritis (JIA), or juvenile rheumatoid arthritis (JRA). In some embodiments, the subject has rheumatoid synovitis or significant systemic involvement secondary to RA (including but not limited to vasculitis, pulmonary fibrosis or Felty's syndrome). In some embodiments, the subject is positive for anti-citrullinated protein antibodies (ACPA). In some embodiments, the subject is positive for anti-PAD4 autoantibodies. Typically, such anti-PAD4 autoantibodies activate PAD4.

Rheumatoid arthritis (RA) is a major autoimmune disease the pathobiology of which commonly includes the presence of auto-antibodies including anti-citrullinated protein antibodies (ACPA). PAD4, a post-translational modification enzyme, citrullinates proteins that serve as neo-auto-antigens. These neo-auto-antigens, when presented, result in the production of ACPAs and are recognized by ACPAs to form immune complexes, thus leading to the initiation and progression of the disease. The role of PAD4 in the pathogenesis of RA in an ACPA-independent fashion, for example, is reviewed in Curran A M, Naik P, Giles J T, Darrah E. Nat Rev Rheumatol. 2020 June; 16(6):301-315. The importance of PAD4 in RA has been further supported by several lines of evidence. For example, the PADI gene is identified as a risk locus for RA, as reviewed in Curran A M, Naik P, Giles J T, Darrah E. Nat Rev Rheumatol. 2020 June; 16(6):301-315. Single-nucleotide polymorphorphisms in the PADI4 gene that encodes PAD4 have been identified that contribute to a susceptibility haplotype for RA, for example. (Susuki et al., Nat. Genet. 34: 395-402 (2003).) Epigenetic changes at the promoter region of PADI4 were also found to correlate with RA disease activity and to the level of ACPA in RA subjects. (Kolarz et al., J. Clin. Med. 9: 2049 (2020); Reyes-Castillo et al., Clin. Exp. Immunol. 182: 119-31 (2015).) On a mechanistic level, PAD4 citrullinates various proteins known to be targets of ACPA, which antibodies are used as part of the classification and diagnosis of RA in subjects via the anti-CCP test. In addition to ACPA, anti-PAD4 antibodies are also present in a subset of RA patients and are significantly correlated with increased swollen joint count and RA disease severity. Among these anti-PAD4 antibodies found in RA subjects are antibodies that activate PAD4 in RA subjects. (Halvorsen et al., Ann. Rheum. Dis. 68: 249-52 (2009); Zhao et al., J. Rheumatol. 35: 969-74 (2008); Darrah et al., Sci. Transl. Med. 5: 186ra65 (2013).) As Curran et al. reviews, anti-PAD4 auto-antibodies have been detected in up to 45% patients with RA and shown to be associated with disease activity. Preclinically, deficiency of PAD4 has been shown to ameliorate experimental inflammatory arthritis mouse models (Weri Y. et al., Sci Rep. 2015 Aug. 21; 5:13041; Suzuki A et al., BMC Musculoskelet Disord. 2016 May 5; 17:205; Fukui S. et al., Arthritis Rheumatol. 2022 Feb. 15. doi: 10.1002/art.42093). Data in the Examples herein further shows the activity and efficacy of antibodies provided herein in murine models, including without limitation collagen induced arthritis, acute joint inflammation, and chronic joint inflammation models. In some embodiments, antibodies herein may be used not only for treating a subject with RA, but antibodies may be used for treating a subject at risk for developing RA. For example, in some embodiments, the subject at risk for developing RA has a first-degree relative with RA (i.e., a parent or sibling) and/or presence of anti-citrullinated protein antibodies (ACPA) in serum and/or presence of rheumatoid factor (RF) in serum. For example, presence of anti-citrullinated protein antibodies may be determined in some cases using an anti-CCP test, such as an ELISA test. For example, anti-CCP antibody test positivity (i.e., presence of ACPA) was found in 46% of 340 individuals who did not meet the classification criteria for RA but nonetheless had anti-CCP testing performed, such as due to joint pain or lung disease. Those 46% went on to meet the classification for RA within the subsequent 5 years. (Ford et al., Rheum. Dis. Clin North Am 45: 101-112 (2019). In some cases, such anti-CCP test results may be positive up to 10 years prior to onset of RA symptoms. (See, e.g., Jones et al., Curr. Op. Drug Discov. Dev., 12(5): 616-627 (2009).) PAD4 has been found in synovial fluid and synovial biopsies of RA subjects, along with citrullinated proteins, and it has also been found in NETs generated from neutrophils of RA subjects. It is thought that citrullination of these target proteins such as fibrinogen, vimentin, and histones, is promoted in the subclinical phase of RA development, and may be triggered by factors such as cigarette smoking (which is known to increase PAD expression in lung tissue) and periodontal disease (via PAD activity of the oral microbe P. gingivalis). (Curran et al.; Chang et al., Arthitis Res. Ther. 7: R268 (2005); Smolen et al., Nat. Rev. Dis. Priers 4: 18001 (2018).) Accordingly, in some cases, a subject at risk for developing RA has a history of smoking (e.g., cigarettes, cigars) or of using tobacco products (e.g., chewing tobacco), and/or has periodontal disease.

In some such cases, the subject at risk for developing RA does not have clinical symptoms of arthritis. In some embodiments, however, the subject shows subclinical symptoms of arthritis, such as joint inflammation visible by imaging, such as ultrasound or magnetic resonance imaging (MM), presence of ACPA via an anti-CCP test, presence of rheumatoid factor (RF), or an SNP or other genetic alteration in the PADI4 gene or its promoter region characteristic of subjects with RA. In some cases, the subject has such subclinical symptoms along with one or both of a first-degree relative with RA and serum ACPA or serum rheumatoid factor (RF). In other cases, the subject has been diagnosed with arthralgia or undifferentiated arthritis. For example, “arthralgia” herein refers to symptoms of pain or aching in at least one joint, such as an ankle, toe, shoulder, elbow, wrist, knee, hip, or one or more joints of the hand, fingers, or spine. A subject with arthralgia may also have tenderness, redness, warmth, loss of mobility, stiffness, weakness, numbness and/or tingling in one or more joints. “Undifferentiated arthritis” refers to diagnosis of arthritis in a subject for which the type of arthritis, such as RA or osteoarthritis, is not specified or cannot be determined. In the case of a subject with arthralgia or undifferentiated arthritis, the subject may also have one or more of a first-degree relative with RA, ACPA in serum, RF in serum, and subclinical joint inflammation (e.g., by ultrasound or MRI). For example, in such subjects at risk of developing RA, the treating may comprise, for example, lessening effects of one or more present clinical symptoms and/or one or more present sub-clinical symptoms. In some cases, the antibody herein may be administered in a method of preventing onset or recurrence of RA in a subject that is at risk of developing RA.

In some embodiments, an RA subject or a subject at risk of developing RA has a comorbidity. In some embodiments, the comorbidity is a lung disorder such as interstitial lung disease (ILD), pleural effusion, cricoarytenoiditis, constrictive or follicular bronchiolitis bronchiectasis, pulmonary vasculitis, or pulmonary hypertension. (See, e.g., S. Kadura & G. Raghu, Eur. Respiratory Rev. 30: 210011 (2021).) In some cases, the lung disorder is a parenchymal lung disease (e.g., pneumonia), an airway disease (e.g., cricoarytenoiditis), or a pleural disease (e.g., pleural effusion). (S. Kadura & G. Raghu.) In some embodiments, the comorbidity is a lung disorder characterized by inflammation and/or scarring (fibrosis) of the lung, such as interstitial lung disease (ILD), also known as pulmonary fibrosis. For instance, Examples 20 and 25 herein, using acute lung inflammation models, showed that several anti-PAD4 antibodies herein inhibited PAD4 function in inflamed lungs, as demonstrated by decreased citrullination of histone H3 and/or ITIH4 in broncheoalveolar lavage fluid (BALF) collected from the inflamed lungs (see, e.g., Table 17).

In other embodiments, the subject has not been diagnosed with RA, but has a lung disorder, such as a disorder characterized by inflammation and/or scarring of the lung, such as interstitial lung disease (ILD), also known as pulmonary fibrosis, or has a parenchymal lung disease (e.g., pneumonia), an airway disease (e.g., cricoarytenoiditis), or a pleural disease (e.g., pleural effusion), or has interstitial lung disease (ILD), pleural effusion, cricoarytenoiditis, constrictive or follicular bronchiolitis bronchiectasis, pulmonary vasculitis, or pulmonary hypertension. For example, ILD can also be a comorbidity with other autoimmune diseases such as scleroderma, dermatomyositis and polymyositis, mixed connective tissue disease, Sjogren's syndrome, and sarcoidosis, as well as result from certain infectious diseases such as pneumonia, or exposure to certain drugs or harmful substances such as asbestos, or can result from uncontrolled gastroesophageal reflux.

In some embodiments, the autoimmune disorder comprises or is a rheumatic autoimmune disease other than RA. PAD4 gene polymorphism, for example, is not only associated with RA, but is also associated with lupus, such as systemic lupus erythematosus (SLE), cutaneous lupus erythematosus, and lupus nephritis. For example, Padi4 −/− individuals have been found to display decreased autoantibodies, type I IFN responses, immune cell activation, vascular dysfunction, and NET immunogenicity. Human T cells express both PAD4 and PAD2, and when exposed to either PAD2 or PAD4 inhibitors, display abrogation of Th1 polarization. In the case of lupus nephritis, for example, Padi4 knock-out mice showed significant improvements in proteinuria progression compared with wild-type mice, decreased neutrophil infiltration in kidneys, and reduced phosphorylation of p38 MAPK and lower expression of JNK-associated leucine zipper protein (JLP), a p38 MAPK scaffold protein. (See, for example, Massarenti et al., Scand. J. Rheumatol. 48(2): 133-140 (2019); Y. Liu et al., JCI Insight 3(23): e124729; N. Hanata et al., Front. Immunol. 11: 1095 (2020).)

NETosis is associated with the pathophysiology of lupus and other autoimmune and renal diseases, including, for instance systemic lupus erythematosus, vasculitis (e.g., ANCA-associated vasculitis), antiphospholipid antibody syndrome, type 1 diabetes mellitus, and renal inflammatory diseases (gomerulophritides, e.g., proliferative glomerulonephritis and non-proliferative gromerulonephritis), and is also associated with the pathophysiology of cancer. (See, e.g., Li et al., Molecular Cancer Therapeutics, 19: 1530-38 (2020), Teijeira et al. Immunity: 56, 856-871 (2020), Gupta, S. and Kaplan, M. J. Nat Rev Nephrol 12(7):402-413 (2016).) NETs are extracellular web-like structures composed of chromatin backbone and various peptides and proteins that are formed by neutrophils in response to various stimuli in a process called NETosis. NETosis has been found to involve the citrullination of histones, such as histone H3, which requires PAD4 activity. In vasculitis, for instance, NETosis is a key driver of disease. (See, for example, B. Arneth et al., Int. J. Med. Sci. 18: 1532-40 (2021); J M Berthelot et al., Joint Bone Spine 84(3): 255-262 (2017); Z L Wang et al., Beijing Da Xue Xue Bao Yi Xue Ban 46(2): 200-6 (2014).) PAD4 antibodies provided herein have been found to inhibit NETosis and citrullination of H3 (see Examples) and can be used to treat or prevent onset or recurrence of NETosis associated disease. In some embodiments, the disease is cancer (e.g., a cancer disclosed herein), or an autoimmune disease, such as, e.g., lupus (e.g., systemic lupus erythematosus), vasculitis (e.g., ANCA-associated vasculitis), antiphospholipid antibody syndrome, type 1 diabetes mellitus, inflammatory bowel disease (IBD) (e.g., ulcerative colitis and Crohn's disease), and cystic fibrosis, or a renal disease such as renal inflammatory disease (e.g., proliferative glomerulonephritis and non-proliferative gromerulonephritis). Accordingly, methods herein include methods of inhibiting NETosis or METosis in vivo in a subject, or in vitro, comprising administering an effective amount of an antibody herein.

Furthermore, in thrombosis, either absence of PAD4 or inhibition of PAD4 has been shown to abrogate thrombus formation induced by heparin, for example. In addition, injection of recombinant human PAD4 in vivo induces the formation of von Willebrand factor platelet strings in mesenteric venules, which is dependent on PAD4 enzymatic activity. There is also a reduction of endogenous ADAMTS13 activity in the plasma of wild-type mice injected with recombinant human PAD4. Administration of recombinant human PAD4 also decreased time to vessel occlusion and significantly reduced thrombus embolization. (See, for example, J. Perdomo et al., Nature Commun. 10(1): 1322 (2019); N. Sorvillo et al., Circulation Res. 125(5): 507-519 (2019).)

In addition, abnormal PAD4 activity is known to be associated with autoimmune disorders in addition to RA, such as multiple sclerosis (MS), autoimmune encephalomyelitis, obstructive nepropathy, Alzheimer's disease (AD), and inflammatory bowel disease (IBD) (e.g., ulcerative colitis and Crohn's disease), as well as ankylosing spondylitis, osteoarthritis, glaucoma, Scrapie, and HIV/AIDS. For example, elevated levels of PAD enzymes and/or citrullinated proteins have been found in all of those conditions. (See, e.g., Chumanevich et al., Am. J. Physiol. Gastrointest. Liver Physiol. 300(6): G929-G938 (2011); Jones et al., Curr. Op. Drug Discov. Dev., 12(5): 616-627 (2009).) For example, in MS, both in patients and in animal models, myelin basic protein was found to be abnormally deiminated and elevated levels of PAD4 were observed; while in Marburg MS, a particularly severe form of MS, levels of citrullinated myelin basic protein were found to be very high. (See, e.g., Jones et al., Curr. Op. Drug Discov. Dev., 12(5): 616-627 (2009).) Moreover, deamination of PAD substrates has been suggested to occur in response to TNF-alpha signaling, while anti-TNF-alpha antibodies have been used as treatments for various autoimmune conditions such as RA and IBD, suggesting that the elevated PAD activity may result from uncontrolled TNF-alpha signaling. (See Chumanevich et al., supra.) Chumanevich and colleagues, for example, found elevated levels of PAD4 in a colitis model and showed that a small molecule PAD inhibitor could be used to treat colitis in a dextran sulfate sodium (DSS)-induced murine colitis model. (Id.) Thus, in some embodiments, the autoimmune disorder comprises IBD. In some embodiments, the autoimmune disorder comprises colitis, such as ulcerative colitis, Crohn's disease, gluten-sensitive enteropathy, or Whipple's disease.

In some embodiments, the autoimmune disorder comprises lupus, such as systemic lupus erythematosus, cutaneous lupus erythematosus, or lupus nephritis. In some embodiments, the autoimmune disorder comprises vasculitis. Exemplary types of vasculitis include Bechet's Disease, Buerger's Disease (Thromboangiitis Obliterans), eosinophilic granulomatosis with polyangiitis (EGPA; formerly known as Churg Strauss), cryoglobulinemia, giant cell arteritis (temporal arteritis), Henoch-Schönlein purpura (HSP; IgA vasculitis), microscopic polyangiitis, polyarteritis nodosa, polymyalgia rheumatica, rheumatoid vasculitis, Takayasu's arteritis, granulomatosis with polyangiitis (GPA; formerly known as Wegener's), ANCA-associated vasculitis (such as PR3-ANCA associated vasculitis or MPO-ANCA associated vasculitis), hypersensitivity vasculitis, isolated aortitis, central nervous system vasculitis, primary angiitis of the central nervous system (PACNS), Kawasaki Disease, urticarial vasculitis, drug-induced vasculitis, relapsing polychondritis (RP). In some embodiments, the autoimmune disorder comprises thrombosis. In some cases, the autoimmune disorder comprises arthritis, such as, e.g., acute arthritis, chronic arthritis, gout or gouty arthritis, acute immunological arthritis, chronic inflammatory arthritis, degenerative arthritis, type II collagen-induced arthritis, infectious arthritis, septic arthritis, Lyme arthritis, proliferative arthritis, psoriatic arthritis, Still's disease, vertebral arthritis, osteoarthritis, arthritis chronica progrediente, arthritis deformans, polyarthritis chronica primaria, reactive arthritis, menopausal arthritis, estrogen-depletion arthritis, ankylosing spondylitis, or rheumatoid spondylitis. In some cases, the autoimmune disorder comprises multiple sclerosis (MS), such may include: primary progressive multiple sclerosis (PPMS), relapsing-remitting multiple sclerosis (RRMS), secondary progressive multiple sclerosis (SPMS), and progressive relapsing multiple sclerosis (PRMS). In some cases, the autoimmune disorder comprises systemic sclerosis (scleroderma), idiopathic inflammatory myopathy (such as, e.g., dermatomyositis, polymyositis, necrotizing autoimmune myopathy, or sporadic inclusion body myositis), Sjogren's syndrome, sarcoidosis, autoimmune hemolytic anemia, immune pancytopenia, paroxysmal nocturnal hemoglobinuria, autoimmune thrombocytopenia (such as, e.g., idiopathic thrombocytopenic purpura, immune-mediated thrombocytopenia, acute thrombocytopenic purpura, chronic thrombocytopenic purpura), thyroiditis (such as, e.g., Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), a demyelinating disease of the central and/or peripheral nervous system (such as, e.g., multiple sclerosis, idiopathic demyelinating polyneuropathy or Guillain-Barre syndrome, or chronic inflammatory demyelinating polyneuropathy), a hepatobiliary disease (such as, e.g., infectious hepatitis (e.g., hepatitis A, B, C, D, E or other non-hepatotropic virus), autoimmune chronic active hepatitis, primary biliary cirrhosis, granulomatous hepatitis, or sclerosing cholangitis), inflammatory bowel disease (IBD)(such as, e.g., ulcerative colitis, Crohn's disease, gluten-sensitive enteropathy, or Whipple's disease), an autoimmune or immune-mediated skin disease (such as, e.g., a bullous skin disease, erythema multiforme, contact dermatitis, or psoriasis), an allergic disease (such as, e.g., asthma, allergic rhinitis, atopic dermatitis, food hypersensitivity, or urticaria), an immunologic disease of the lung (such as, e.g., eosinophilic pneumonia, idiopathic pulmonary fibrosis or hypersensitivity pneumonitis), a transplantation associated disease (such as, e.g., graft rejection or graft-versus-host-disease), fibrosis (such as, e.g., kidney fibrosis or hepatic fibrosis), cardiovascular disease, including atherosclerosis and coronary artery disease, cardiovascular events associated with chronic kidney disease, myocardial infarction, and congestive heart failure, diabetes, including type II diabetes, Bronchiolitis obliterans with organizing pneumonia (BOOP), or hemophagocytic syndrome, macrophage activation syndrome, sarcoidosis, or periodontitis). In some cases, the autoimmune disorder comprises a methotrexate-resistant autoimmune disorder, such as methotrexate-resistant RA, lupus, vasculitis, thrombosis, MS, or the like. In some cases, the autoimmune disorder comprises a renal disease, such as a renal inflammatory disease such as, e.g., kidney fibrosis, chronic kidney disease, proliferative glomerulonephritis, or non-proliferative gromerulonephritis.

In some cases, the subject has a disorder that is associated with one or more of NETosis, METosis, presence of anti-citrullinated protein antibodies (ACPA), increased PAD4 expression, or increased PAD4 activity such as increased citrullination of polypeptides. In some cases, the disorder comprises acid-induced lung injury, acne (PAPA), acute lymphocytic leukemia, acute respiratory distress syndrome, Addison's disease, adrenal hyperplasia, adrenocortical insufficiency, ageing, AIDS, alcoholic hepatitis, alcoholic liver disease, allergen induced asthma, allergic bronchopulmonary, aspergillosis, allergic conjunctivitis, alopecia, Alzheimer's disease, amyloidosis, amyotrophic lateral sclerosis, weight loss, angina pectoris, angioedema, anhidrotic ecodermal dysplasia-ID, ankylosing spondylitis, anterior segment, inflammation, antiphospholipid syndrome, aphthous stomatitis, appendicitis, arthritis, asthma, atherosclerosis, atopic dermatitis, autoimmune diseases, autoimmune hepatitis, bee sting-induced inflammation, Bechet's disease, Bechet's syndrome, Bells Palsy, berylliosis, Blau syndrome, bone pain, bronchitis, bronchiolitis, burns, bursitis, cardiac hypertrophy, carpal tunnel syndrome, catabolic disorders, cataracts, cerebral aneurysm, chemical irritant-induced inflammation, chorioretinitis, chronic heart failure, chronic lung disease of prematurity, chronic lymphocytic leukemia, chronic obstructive pulmonary disease, colitis, complex regional pain syndrome, connective tissue disease, COPD, corneal ulcer, Crohn's disease, cryopyrin-associated periodic syndromes, cryptococcosis, cystic fibrosis, deficiency of the interleukin-1-receptor antagonist (DIRA), dermatitis, dermatitis endotoxemia, dermatomyositis, diffuse intrinsic pontine glioma, dry eye disease, endometriosis, endotoxemia, epicondylitis, erythroblastopenia, familial amyloidotic polyneuropathy, familial cold urticarial, familial Mediterranean fever, fetal growth retardation, glaucoma, glomerular disease, glomerular nephritis, gout, gouty arthritis, graft-versus-host disease, gut diseases, head injury, headache, hearing loss, heart disease, hemolytic anemia, Henoch-Scholein purpura, hepatitis, hereditary periodic fever syndrome, herpes zoster and simplex, HIV-1, Hodgkin's disease, Huntington's disease, hyaline membrane disease, hyperammonemia, hypercalcemia, hypercholesterolemia, hyperimmunoglobulinemia D with recurrent fever (HIDS), hypoplastic and other anemias, hypoplastic anemia, idiopathic thrombocytopenic purpura, incontinentia pigmenti, infectious mononucleosis, inflammatory bowel disease, inflammatory lung disease, inflammatory neuropathy, inflammatory pain, insect bite-induced inflammation, iritis, irritant-induced inflammation, ischemia/reperfusion, juvenile rheumatoid arthritis, keratitis, kidney disease, kidney injury caused by parasitic infections, kidney injury caused by parasitic infections, kidney transplant rejection prophylaxis, leptospirosis, Lewy body dementia, Loeffler's syndrome, lung injury, lupus, lupus nephritis, meningitis, mesothelioma, mixed connective tissue disease, Muckle-Wells syndrome (urticaria deafness amyloidosis), multiple sclerosis, multiple system atrophy, muscle wasting, muscular dystrophy, myasthenia gravis, myocarditis, mycosis fungoides, myelodysplastic syndrome, myositis, nasal sinusitis, necrotizing enterocolitis, neonatal onset multisystem inflammatory disease (NOMID), nephrotic syndrome, neuritis, neuropathological diseases, non-allergen induced asthma, obesity, ocular allergy, optic neuritis, organ transplant, osteoarthritis, otitis media, Paget's disease, pain, pancreatitis, Parkinson's disease, pemphigus, pericarditis, periodic fever, periodontitis, peritoneal endometriosis, pertussis, pharyngitis and adenitis (PFAPA syndrome), plant irritant-induced inflammation, pneumonia, pneumonitis, pneumocystis infection, poison ivy or urushiol oil-induced inflammation, polyarteritis nodosa, polychondritis, polycystic kidney disease, polymyositis, psoriasis, psychosocial stress diseases, pulmonary disease, pulmonary hypertension, pulmonary fibrosis, pyoderma gangrenosum, pyogenic sterile arthritis, renal disease, retinal disease, rheumatic carditis, rheumatic disease, rheumatoid arthritis, sarcoidosis, seborrhea, sepsis, severe pain, sickle cell, sickle cell anemia, silica-induced disease, Sjogren's syndrome, skin diseases, sleep apnea, spinal cord injury, spondylitis, spondyloarthropathy, Stevens-Johnson syndrome, stroke, subarachnoid hemorrhage, sunburn, temporal arteritis, tenosynovitis, thrombocytopenia, thyroiditis, tissue transplant, TNF receptor associated periodic syndrome (TRAPS), toxoplasmosis, transplant, traumatic brain injury, tuberculosis, type 1 diabetes, type 2 diabetes, ulcerative colitis, urticarial, uveitis (including nongranulamotous uveitis and granulomatous uveitis), wound healing, Wegener's granulomatosis, interstitial lung disease, psoriatic arthritis, juvenile idiopathic arthritis, antineutrophil cytoplasmic antibody (ANCA)-associated vasculitis, antiphospholipid antibody syndrome, deep vein thrombosis, fibrosis, Alzheimer's, scleroderma or CREST syndrome.

In some embodiments, the disorder is cancer. For example, neutrophil inflammation, neutrophil extracellular traps (NET), and/or monocyte extracellular traps (MET) have been identified in cancers, and are associated with poorer prognosis. (See, e.g., Li et al., Molecular Cancer Therapeutics, 19: 1530-38 (2020).) In some embodiments, the antibodies described herein may reduce extracellular trap forming neutrophils and/or inhibit NETosis, reduce extracellular trap forming monocytes and/or inhibit METosis, and/or attenuate cancer growth. For example, studies have shown that PAD4-catalyzed NET formation is upregulated in multiple tumors, and that PAD4 is overexpressed in a variety of cancers. (H. Chen et al., Cell Mol. Biol. Lett 26:9 (2021).) A small molecule PAD4 inhibitor was also shown to inhibit tumor growth and to inhibit histone H3 citrullination in a cancer model. (See Id.) Thus, in some embodiments, antibodies herein inhibit NETosis and/or METosis in a cancer subject. In addition, PAD4 has been reported to be highly expressed in certain tumor tissues and in blood samples of cancer patients. (See, e.g., Wang et al., Biomedicine & Pharmacotherapy 153: 113289 (2022).) PAD4 has also been reported to promote radioresistance, survival, migration and invasion of cancer cells. (Chen et al Cell Mol Biol Lett (2021) 26:9.) In some embodiments, the antibodies may inhibit the growth of at least one tumor in the patient and/or reduce the volume of at least one tumor in the patient. In some embodiments, the antibodies increase radiosensitivity or reduce radioresistance of a tumor.

In some embodiments, the cancer is a cancer that is typically responsive to immunotherapy. In some embodiments, the cancer is a cancer that is not typically responsive to immunotherapy. In some embodiments, the cancer comprises a solid tumor. In some embodiments, the cancer comprises a blood malignancy (liquid tumor).

In some embodiments, the cancer is carcinoma, lymphoma, blastoma, sarcoma, or leukemia. In some embodiments, the cancer is squamous cell cancer, small-cell lung cancer, pituitary cancer, esophageal cancer, astrocytoma, soft tissue sarcoma, non-small cell lung cancer (including squamous cell non-small cell lung cancer), adenocarcinoma of the lung, squamous carcinoma of the lung, cancer of the peritoneum, hepatocellular cancer, gastrointestinal cancer, pancreatic cancer, glioblastoma squamous cell carcinoma, small-cell lung cancer (SCLC), non-small cell lung cancer, squamous non-small cell lung cancer (NSCLC), non-squamous NSCLC, glioma, gastrointestinal cancer, renal cancer (e.g., clear cell renal carcinoma), ovarian cancer, liver cancer, colorectal cancer, endometrial cancer, kidney cancer (e.g., renal cell carcinoma (RCC)), prostate cancer (e.g., hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer (including triple-negative breast cancer, ER positive breast cancer, ER negative breast cancer, node positive breast cancer, and node negative breast cancer), colon carcinoma, and head and neck cancer (or carcinoma), gastric cancer, germ cell tumor, pediatric sarcoma, sinonasal natural killer/T-cell lymphoma, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, T-cell lymphoma, an environmentally-induced cancer (e.g., a cancer induced by asbestos, a virus-related cancer or a cancer of viral origin (e.g., human papilloma virus (HPV-related or -originating tumors)), a hematologic malignancy derived from either of the two major blood cell lineages (i.e., the myeloid cell line (which produces granulocytes, erythrocytes, thrombocytes, macrophages and mast cells) or lymphoid cell line (which produces B, T, NK and plasma cells), such as, e.g., a leukemia, lymphoma, or myeloma (of any type), e.g., acute, chronic, lymphocytic and/or myelogenous leukemia, such as acute leukemia (ALL), acute myelogenous leukemia (AML), chronic lymphocytic leukemia (CLL), and chronic myelogenous leukemia (CIVIL), undifferentiated AML (MO), myeloblastic leukemia (ML), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; a lymphoma, such as Hodgkin's lymphoma (HL), non-Hodgkin's lymphoma (NHL), B cell hematologic malignancy, e.g., B-cell lymphoma, T-cell lymphoma, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio-immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukemia (T-Lbly/T-ALL), peripheral T-cell lymphoma, lymphoblastic lymphoma, post-transplantation lymphoproliferative disorder, true histiocytic lymphoma, primary central nervous system lymphoma, primary effusion lymphoma, B cell lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; a hematopoietic tumor of myeloid lineage, a tumor of mesenchymal origin, including fibrosarcoma and rhabdomyoscarcoma; seminoma, teratocarcinoma, a tumor of the central or peripheral nervous system, such as astrocytoma, schwannoma; a tumors of mesenchymal origin, such as fibrosarcoma, rhabdomyoscaroma, and osteosarcoma; or another tumor, such as melanoma, xeroderma pigmentosum, keratoacanthoma, seminoma, thyroid follicular cancer, teratocarcinoma, a hematopoietic tumor of lymphoid lineage, for example a T-cell or B-cell tumor, such as a T-cell disorder such as T-prolymphocytic leukemia (T-PLL), such as of the small cell or cerebriform cell type; a large granular lymphocyte leukemia (LGL) of the T-cell type; a/d T-NHL hepatosplenic lymphoma; peripheral/post-thymic T cell lymphoma (pleomorphic or immunoblastic subtype); angiocentric (nasal) T-cell lymphoma; cancer of the head or neck, renal cancer, rectal cancer, cancer of the thyroid gland; acute myeloid lymphoma, or any combinations of said cancers. The methods described herein can also be used for treatment of metastatic cancers, unresectable cancers, refractory cancers (e.g., cancers refractory to previous immunotherapy, e.g., with an anti-CTLA-4 or anti-PD-1 antibody), and/or recurrent cancers.

In certain embodiments, an antibody described herein is administered to patients having a cancer that has exhibited an inadequate response to, or progressed on, a prior treatment, such as a standard of care treatment, e.g., a prior treatment with an immuno-oncology or immunotherapy drug. In some embodiments, the cancer is refractory or resistant to a prior treatment, either intrinsically refractory or resistant (e.g., refractory to an immune checkpoint inhibitor such as a PD-1 pathway antagonist), or a resistance or refractory state is acquired. For example, an antibody described herein may be administered to subjects who are not responsive or not sufficiently responsive to a first therapy, such as a standard of care therapy, or who have disease progression following treatment, e.g., with chemotherapy or with an immune checkpoint inhibitor such as a PD-1 pathway antagonist, either alone or in combination with another therapy (e.g., with an anti-PD-1 pathway antagonist therapy). In other embodiments, an antibody described herein is administered to patients who have not previously received (i.e., been treated with an immune checkpoint inhibitor, e.g., a PD-1 pathway antagonist. In some embodiments, an anti-PD1 pathway antagonist is a small molecule anti-PD-1, anti-PD-L1, or anti-CTLA4 antagonist, or is an anti-PD-1, anti-PD-L1, or anti-CTLA4 antibody, such as nivolumab, pembrolizumab, cemiplimab, atezolizumab, dostarlimab, durvalumab, avelumab, or ipilimumab.

In some embodiments, the antibodies herein are used for treating an infectious disease. For example, occurrence of NETosis has also been found in various infections. (See, e.g., Li et al., Molecular Cancer Therapeutics, 19: 1530-38 (2020).)

Infectious diseases that may be treated herein include, for example, viral diseases (including AIDS (HIV infection), hepatitis (A, B, C, D, and E), and herpes), bacterial infections, fungal infections, protozoal infections and parasitic infections. Examples of pathogenic infections include, but are not limited to, HIV, Hepatitis (A, B, & C), Influenza, Herpes (e.g., VZV, HSV-1, HAV-6, HSV-II, CMV, Epstein Barr virus), Giardia, Malaria, Leishmania, Staphylococcus aureus, Pseudomonas aeruginosa, adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus, arboviral encephalitis virus, chlamydia, rickettsial bacteria, mycobacteria, staphylococci, streptococci, pneumonococci, meningococci and gonococci, Klebsiella, Proteus, Serratia, Pseudomonas, Legionella, diphtheria, Salmonella, bacilli, cholera, tetanus, botulism, anthrax, plague, leptospirosis, Lyme disease bacteria, Candida (albicans, krusei, glabrata, tropicalis, etc.), Cryptococcus neoformans, Aspergillus (fumigatus, niger, etc.), Genus Mucorales (mucor, absihlorambzopus), Sporothrix schenkii, Blastomyces dermatitidis, Paracoccidioides brasiliensis, Coccidioides immitis and Histoplasma capsulatum, Entamoeba histolytica, Balantidium coli, Naegleria fowleri, Acanthamoeba sp Giardia Zambia, Cryptosporidium sp., Pneumocystis carinii, Plasmodium vivax, Babesia microti, Trypanosoma brucei, Trypanosoma cruzi, Leishmania donovani, Toxoplasma gondii, and Nippostrongylus brasiliensis. In some cases, the infectious disease is caused by a viral pathogen. In other cases, it is caused by a bacterial pathogen.

In some cases, an anti-PAD4 antibody herein may be used to inhibit NETosis and/or METosis in a subject, or in a biological sample. Inhibition of NETosis and/or METosis can be assessed, for instance, using a biological sample such as a whole blood sample, serum sample, plasma sample, synovial fluid sample, lung fluid sample, tissue sample (e.g., joint tissue sample, lung tissue sample), tumor sample, or other biological sample containing neutrophils, monocytes, and/or macrophages susceptible to NETosis or METosis. Accordingly, the disclosure includes use of an anti-PAD4 antibody disclosed herein for inhibiting NETosis and/or METosis in a subject, or in the preparation of a medicament for inhibiting NETosis and/or METosis in a subject, as well as a method of inhibiting NETosis and/or METosis either in a subject or in vitro in a biological sample, comprising administering an effective amount of an anti-PAD4 antibody as described herein.

In some cases, an anti-PAD4 antibody disclosed herein may be used to inhibit citrullination in a subject, or in a biological sample. Inhibition of citrullination in a subject can be assessed in vitro in a biological sample from the subject. The biological sample can be, for instance, a whole blood, serum, plasma, blood supernatant, synovial fluid, tissue (e.g., joint tissue, lung tissue) or tumor sample. Accordingly, the disclosure includes use of an anti-PAD4 antibody disclosed herein for inhibiting citrullination in a subject, or in the preparation of a medicament for inhibiting citrullination in a subject, as well as a method of inhibiting citrullination either in a subject, comprising administering an effective amount of an anti-PAD4 antibody disclosed herein to the subject. In some cases, citrullination (e.g., of one or more proteins or of specific citrullination sites on proteins) in a subject is inhibited in comparison to citrullination in the subject prior to anti-PAD4 antibody administration. This can be assessed, for example, by comparing citrullination in a biological sample from the subject obtained after antibody administration to the subject to citrullination in a biological sample obtained before antibody administration, or in comparison to a control biological sample, for instance. The disclosure also relates to inhibiting citrullination of a biological sample, comprising administering an effective amount of an anti-PAD4 antibody to the sample. In some cases, citrullination (e.g., of one or more proteins or of specific citrullination sites on proteins) in a biological sample that has been exposed to the anti-PAD4 antibody is inhibited in comparison to that in a control sample (e.g., a control sample that has not been exposed to the anti-PAD4 antibody, for instance, an untreated or pre-treatment control sample or a control sample that has been exposed to a control anti-idiotypic antibody).

Citrullination can be assessed, for instance, by assessing citrullination of a protein, or a peptide fragment thereof, in a biological sample. In some embodiments, the protein is a protein listed in Table 20. In some embodiments, the peptide is a peptide listed in Table 20. In some cases, more than one protein or peptide fragment may be assessed (e.g., more than one protein and/or more than one peptide fragment from the same protein). In some cases, one or more of the proteins or peptides listed in Table 20 may be used for determining citrullination. Citrullination can be assessed at a particular citrullination site, for instance, a citrullination site identified in Table 20 or a corresponding site. As used here, a “corresponding site” refers to a corresponding citrullination site that can be determined, for instance, using a sequence alignment. For example, a naturally occurring variant or isoform of a protein (e.g., a protein listed in Table 20) can be aligned with a protein sequence referenced in Table 20 to identify a citrullination site that corresponds to an identified citrullination site. In some embodiments, the citrullination is assessed using mass spectrometry (e.g., using LC/MS). Citrullination can be assessed in some embodiments using mass spectrometry to measure the concentration of a citrullinated protein or peptide and the concentration of the corresponding total protein or peptide (including modified and unmodified forms of the applicable protein). These concentrations can be measured, for instance, using mass spectrometry. These concentrations can, in some embodiments, be expressed as a citrullination ratio, which is a ratio of the concentration of citrullinated protein (or the concentration of a citrullinated peptide from the protein) to the concentration of the corresponding total protein. In some embodiments, citrullination is assessed by subjecting a biological sample to enzymatic digestion and assessing the concentration of a citrullinated peptide (such as a peptide listed in Table 18 or 20 herein) in the sample and the concentration of the corresponding total protein in the sample. In some embodiments, the concentration of corresponding total protein is measured by measuring the concentration of a signature peptide from the protein, which is a peptide that is not modified and therefore represents the total concentration of the corresponding protein (including any modified and unmodified forms of the protein). Comparison of the citrullination ratios assessed in two samples (e.g., pre and post-treatment samples, or in a treated sample and a control sample) can be used to assess inhibition of citrullination by an anti-PAD4 antibody. A lower citrullination ratio in a sample subjected to treatment with the anti-PAD4 antibody (e.g., in a sample from a subject subjected to treatment with the anti-PAD4 antibody) indicates inhibition of citrullination by the anti-PAD4 antibody. In some cases, citrullination can be assessed in two or more proteins or peptide fragments thereof, such as two or more of the proteins or peptide fragments provided in Table 18 or 20 herein.

TABLE 20
Proteins, peptides and specific citrullination sites that can be citrullinated by PAD4
and inhibited by an anti-PAD4 antibody disclosed herein

| | | | | Amino | |
|---|---|---|---|---|---|
| | | | | acids | |
| | | | Peptide fragment | (where | |
| | | | of protein with | the | |
| | | | citrullination | peptide is | |
| | | | site boldfaced and | in the | |
| Protein | | | underlined | protein | Citrullination |
| (abbreviation) | Abbreviation | UniProt ID | (SEQ ID NO) | sequence) | site |
| Proteoglycan 4 | PRG4 | Q92954 | AITTRSGQTLSK | 1387-1398 | R1391 of |
| | | | (SEQ ID NO: 236) | | Q92954 |
| fibrinogen alpha | FGA | P02671 | ESSSHHPGIAEFPSRGK | 559-575 | R573 of |
| chain | | | (SEQ ID NO: 241) | | P02671 |
| fibrinogen alpha | FGA | P02671 | QFTSSTSYNRGDSTFESK | 582-600 | R591 of |
| chain | | | (SEQ IN NO: 237) | | P02671 |
| complement C3 | C3 | P01024 | SGQSEDRQPVPGQQMTLK | 567-584 | R573 of |
| | | | (SEQ ID NO: 242) | | P01024 |
| complement C3 | C3 | P01024 | ASHLGLARSNLDEDIIAEENIVSR | 741-764 | R748 of |
| | | | (SEQ ID NO: 243) | | P01024 |
| inter-alpha-trypsin | ITIH4 | Q14624 | QLGLPGPPDVPDHAAYHPFRR | 669-689 | R688 of |
| inhibitor heavy chain | | | (SEQ ID NO: 238) | | Q14624 |
| H4 | | | | | |
| protein AMBP | AMBP | P02760 | GPCRAFIQLWAFDAVK | 294-309 | R297 of |
| | | | (SEQ ID NO: 239) | | P02760 |
| alpha-2- | A2M | P01023 | VGFYESDVMGRGHAR | 705-719 | R715 of |
| macroglobulin | | | (SEQ ID NO: 244) | | P01023 |
| gelsolin | GSN | P06396 | ATASRGASQAGAPQGR | 28-43 | R32 of |
| | | | (SEQ ID NO: 240) | | P06396 |
| haptoglobin | HP | P00738-1 | LRTEGDGVYTLNDK | 58-71 | R59 of |
| | | | (SEQ ID NO: 245) | | P00738-1 |
| serotransferrin | TF | P02787-1 | DLLFRDDTVCLAK | 647-660 | R651 of |
| | | | (SEQ ID NO: 246) | | P02787-1 |

In some embodiments, an anti-PAD4 antibody disclosed herein inhibits citrullination of one or more of proteoglycan 4 (PRG4), fibrinogen alpha chain (FGA), complement C3 (C3), inter-alpha-trypsin inhibitor heavy chain H4 (ITIH4), protein AMBP (AMBP), alpha-2 macroglobulin (A2M), gelsolin (GSN), haptoglobin (HP), or serotransferrin (TF). In some embodiments, the antibody inhibits citrullination at a citrullination site shown in Table 20 or in Table 18, or a corresponding site. In some embodiments, the antibody inhibits citrullination of at least one protein or peptide shown in Table 18. In some embodiments, the antibody inhibits citrullination of the arginine residue found in SEQ ID NO: 216 or SEQ ID NO: 232, for example. In some embodiments, the antibody inhibits citrullination of the arginine residue found in any one or more of SEQ ID NO: 216, SEQ ID NO: 232, or SEQ ID NO: 236-246, for example.

B. Routes of Administration and Carriers

In various embodiments, anti-PAD4 antibodies or anti-PAD4 antibody compositions may be administered in vivo by various routes, including, but not limited to, oral, intra-arterial, parenteral, intranasal, intramuscular, intracardiac, intraventricular, intratracheal, buccal, rectal, intraperitoneal, intradermal, topical, transdermal, and intrathecal, or otherwise by implantation or inhalation. The subject compositions may be formulated into preparations, such as liquid formulations or formulations suitable for injections, inhalations, and the like. Alternatively, in some embodiments, the composition may be provided as a lyophilized powder that may be reconstituted upon addition of an appropriate liquid or carrier, for example, sterile water. The appropriate formulation and route of administration may be selected according to the intended application.

In some embodiments, the administration is intravenous or subcutaneous. In some embodiments, the administration is intravenous. In some embodiments, the administration is subcutaneous.

In various embodiments, compositions comprising anti-PAD4 antibodies are provided in formulations with a wide variety of pharmaceutically acceptable carriers (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof. In various embodiments, compositions comprising anti-PAD4 antibodies may be formulated for injection, including subcutaneous administration, by dissolving, suspending, or emulsifying them with appropriate carriers.

In some embodiments, the carrier is a sterile aqueous solution, e.g., normal saline (e.g. 0.9% w/v sodium chloride) or dextrose in water (e.g., 5% w/v dextrose, also known as D5W). In one embodiment provided herein is a method of making a pharmaceutical composition (e.g. for injection, e.g., for intravenous or subcutaneous injection), the method comprising combining a composition of an anti-PAD4 antibody disclosed herein with a carrier to make the pharmaceutical composition. In some embodiments, the pharmaceutical composition comprises at least 0.25 mg/ml of the antibody. In some embodiments, the pharmaceutical composition comprises 0.25 mg/ml to 100 mg/ml of the antibody, such as from 0.25 mg/ml to 50 mg/ml, from 1 mg/ml to 50 mg/ml, or from 10 mg/ml to 50 mg/ml.

Pharmaceutical compositions may be administered in an amount effective for treatment of the specific indication. Pharmaceutical packs and kits comprising one or more containers, each containing one or more doses of an anti-PAD4 antibody are also provided. In some embodiments, a unit dosage is provided wherein the unit dosage contains a predetermined amount of a composition comprising an anti-PAD4 antibody, with or without one or more additional agents.

The anti-PAD4 antibody compositions may be administered as needed to subjects. Determination of the frequency of administration may be made by persons skilled in the art, such as an attending physician based on considerations of the condition being treated, age of the subject being treated, severity of the condition being treated, general state of health of the subject being treated and the like. In some embodiments, an effective dose of an anti-PAD4 antibody is administered to a subject one or more times. For example, in some embodiments, the anti-PAD4 antibody is administered once per week, once every two weeks, once every three weeks, once every month, once every four weeks, once every six weeks, once every two months, once every eight weeks, once every three months, or once every six months. In some such cases, the anti-PAD4 antibody is administered intravenously or subcutaneously at such a time period. In some cases, the administration is intravenous. In some cases, the antibody is administered for a period of at least six months, or at least one year, or at least two years, or at least three years to a subject, such as at one of the above dosing frequencies, such as intravenously or subcutaneously.

In some cases, the antibody is administered at a dose of 0.5 mg to 1000 mg, intravenously or subcutaneously, such as once every two weeks, once every three weeks, once every month, once every four weeks, once every six weeks, once every two months, once every eight weeks, once every three months, or once every six months. In some such cases, the antibody is administered at a dose of 1 mg to 900 mg, 1 mg to 300 mg, 1 mg to 100 mg, 3 mg to 300 mg, 5 mg to 300 mg.

C. Combination Therapy

Anti-PAD4 antibodies may be administered alone or with other modes of treatment. Such other modes of treatment may be provided before, substantially contemporaneous with, or after administration of an anti-PAD4 antibody. In some embodiments, the other mode of treatment comprises a standard of care treatment for the disease suffered by the patient. The antibodies described herein can be administered in the same composition as at least one additional therapeutic agent, or can be administered separately from at least one additional therapeutic agent. The antibodies described herein can also be chemically linked to additional therapeutic agent in some cases, such as within an antibody-drug conjugate.

For treatment of rheumatoid arthritis, for example, an anti-PAD4 antibody may be administered with one or more other therapeutic agents, for example, a disease-modifying anti-rheumatic drug (DMARD) such as methotrexate (Trexall® or Otrexup®), adalimumab (Humira®), etanercept (Enbrel®), infliximab (Remicade®), hydroxychloroquine (Plaquenil®), sulfasalazine (Azulfidine®), leflunomide (Arava®), abatacept (Orencia®), anakinra (Kineret®), Certolizumab (Cimzia®), golimumab (Simponi®), rituximab (Rituxan®), sarilumab (Kevzara®), tocilizumab (Actemra®), baricitinib (Olumiant®), tofacitinib (Xeljanz®), upadacitinib (Rinvoq®), and Orencia® (abatacept); an non-steroidal anti-inflammatory drug (NSAID) such as ibuprofen (Advil, Motrin, and diclofenac) and naproxen sodium; a COX-2 inhibitor (celecoxib or etoricoxib); a steroid such as prednisolone or prednisone. In some cases, an anti-PAD4 antibody may be administered with one or more of: anti-TNF agents (e.g., anti-TNF antibodies) such as infliximab (Remicade®), adalimumab (Humira®), golimumab (Simponi®), certolizumab (Cimzia®), and etanercept (Enbrel®); glucocorticoids such as prednisone or methylprednisolone; leflunomide (Arava®); azathioprine (Imuran® or Azasan®); JAK inhibitors such as CP 590690; SYK inhibitors such as R788; TYK2 inhibitors such as deucravacitinib (Sotyktu®), anti-IL-6 antibodies; anti-IL-6R antibodies; anti-CD-20 antibodies; anti-CD19 antibodies; anti-GM-CSF antibodies; and anti-GM-CSF-R antibodies. For treatment of autoimmune conditions, anti-PAD4 antibodies may be administered with other therapeutic agents, for example, interferon alpha; interferon beta; anti-Type I interferon receptor antibodies such as anifrolumab (Saphnelo®); prednisone; anti-alpha4 integrin antibodies such as Tysabri®; anti-BAFF/BLyS antibodies such as belimumab (Benlysta®); anti-CD20 antibodies such as Rituxan® (rituximab); calcineurin inhibitors such as cyclosporin or voclosporin (Lupkynis®); complement inhibitors such as eculizumab (Soliris®) or avacopan (Tavneos®); mycophenolate mofetil (CellCept®) or mycophenolate sodium (MyFortic®); cyclophosphamide (Cytoxan®); FTY720 (fingolimod, e.g., Gilenya®); and Cladribine® (Leustatin). In some cases, the anti-PAD4 antibody may be administered with methotrexate. In other cases, the anti-PAD4 antibody is administered in the absence of another drug. Anti-PAD4 antibodies may be administered along with other treatments considered the standard of care for the autoimmune disorder, for example, or may be added to or may follow another treatment regime, for example, if the treatment regime has been unsuccessful at meeting standard clinical treatment goals or there is desire for improvement of the treatment regime.

For the treatment of lupus, for example, anti-PAD4 antibodies may be administered with one or more therapeutic agents such as cyclosporine, tacrolimus, cyclophosphamide, azathioprine (Imuran®), mycophenolate (CellCept®), rituximab (Rituxan®), and Belimumab (Benlysta®), steroids (e.g., prednisone or prednisolone), blood pressure medication (e.g., antiotensin-convertin enzyme (ACE) inhibitors, angiotensin II receptor blockers (ARBs)).

For the treatment of vasculitis, for example, anti-PAD4 antibodies may be administered with one or more other therapeutic agents. Non-limiting examples of therapeutic agents include steroids (e.g., prednisone, prednisolone, methylprednisolone, or dexamethasone), methotrexate (Trexall®), azathioprine (Imuran®, Azasan®), mycophenolate (CellCept®), cyclophosphamide, tocilizumab (Actemra®), rituximab (Rituxan®), Avacopan, plasma exchange, mycophenolate mofetil (MMF), azathioprine (AZA), leflunomide (LEF), belimumab, meprolizumab, and omalizumab. For treatment of cancer, anti-PAD4 antibodies may be administered with one or more additional anti-cancer agents, such as an immune checkpoint inhibitor, a chemotherapeutic agent, growth inhibitory agent, radiotoxic agent, immunosuppressive agent, anti-cancer vaccine such as a gene therapy vaccine, anti-angiogenesis agent and/or anti-neoplastic composition. The antibodies described herein can be administered in the same composition as the additional anti-cancer agent, or can be administered separately from the anti-cancer agent. In the latter case (separate administration), the antibody can be administered before, after, or concurrently with the anti-cancer agent, or can be co-administered with other known therapeutic agents. In some embodiments, the combinations may be effectively combined with standard cancer treatments such as including radiation, surgery, and hormone deprivation.

Examples of immune checkpoint inhibitors include molecules that inhibit particular signaling pathways that regulate the immune system. See e.g., Weber (2010) Semin. Oncol. 37:430; Pardoll (2012) Nat. Rev. Cancer 12:252. Immune checkpoint inhibitors, in some embodiments, comprise an antagonist of PD-1, PD-L1, CTLA4, LAG-3, Galectin 1, Galectin 9, CEACAM-1, BTLA, CD25, CD69, TIGIT, CD113, GPR56, VISTA, B7-H3, B7-H4, 2B4, CD48, GARP, PD1H, LAIR1, TIM1, TIM3, TIM4, ILT4, IL-6, IL-10, TGFβ, VEGF, KIR, LAG-3, adenosine A2A receptor, PI3Kdelta, or IDO. In some embodiments, an immune checkpoint inhibitor comprises an agonist of B7-1, B7-2, CD28, 4-IBB (CD137), 4-1BBL, ICOS, ICOS-L, OX40, OX40L, GITR, GITRL, CD27, CD40, CD40L, DR3, CD28H, IL-2, IL-7, IL-12, IL-15, IL-21, IFNα, STING, or a Toll-like receptor agonist such as a TLR2/4 agonist. In some embodiments, an immune checkpoint inhibitor comprises an agent that binds to a member of the B7 family of membrane-bound proteins such as B7-1, B7-2, B7-H2 (ICOS-L), B7-H3, B7-H4, B7-H5 (VISTA), and B7-H6. In some embodiments, an immune checkpoint inhibitor binds to a member of the TNF receptor family or a co-stimulatory or co-inhibitory molecule binding to a member of the TNF receptor family such as CD40, CD40L, OX40, OX40L, GITR, GITRL, CD70, CD27L, CD30, CD30L, 4-1BBL, CD137 (4-IBB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, EDA1, EDA2, TACI, APRIL, BCMA, LTβR, LIGHT, DeR3, HVEM, VEGL/TL1A, TRAMP/DR3, TNFR1, TNFβ, TNFR2, TNFα, 1β2, FAS, FASL, RELT, DR6, TROY, or NGFβ. In some embodiments, an immune checkpoint inhibitor antagonizes or inhibits a cytokine that inhibits T cell activation such as IL-6, IL-10, TGFβ, VEGF. In some embodiments, an immune checkpoint inhibitor comprises an agonist of a cytokine that stimulates T cell activation such as IL-2, IL-7, IL-12, IL-15, IL-21, and IFNα. In some embodiments, the at least one immune stimulating agent comprises an antagonist of a chemokine, such as CXCR2, CXCR4, CCR2, or CCR4. In some embodiments, an immune checkpoint inhibitor comprises an antibody. In some embodiments, an immune checkpoint inhibitor comprises a vaccine, such as a mesothelin-targeting vaccine or attenuated listeria cancer vaccine such as CRS-207.

Exemplary non-limiting example targets of immune checkpoint inhibitors are CTLA-4, PD-1, and PD-L1. Non-limiting examples of such immune checkpoint inhibitors include anti-CTLA4, anti-PD-1, and anti-PD-L1 antibodies, such as, e.g., pembrolizumab (Keytruda®), ipilimumab (Yervoy®), nivolumab (Opdivo®), atezolizumab (Tecentriq®), avelumab (Bavencio®), dostarlimab (Jemperli®), cemiplimab (Libtayo®), and durvalumab (Imfinzi®).

In some embodiments, an antibody herein is administered in combination with at least one chemotherapeutic agent. Non-limiting examples of chemotherapeutic agents that can be administered in methods herein include, but are not limited to, alkylating agents such as thiotepa and Cytoxan®/Neosar® cyclosphosphamide; lenalidomide (Revlimid®); bortezomib (Velcade®); bendamustine (Treanda®); rituximab (Rituxan®); alemtuzumab (Campath®); ofatumumab (Kesimpta®); everolimus (Afinitor®, Zortress®); carfilzomib (Kyprolis™); ifosamade; dexamethasone; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil (Leukeran®), chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, Adriamycin® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine (Fludara®), 6-mercaptopurine, thiamiprine, thioguanine; pyrimidine analogs such as gemcitabine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; chlorambucaxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone′ 2″2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxanes, e.g., Taxol® paclitaxel (Bristol-Myers Squibb Oncology, Princeton, N.J.), Abraxane® Cremophor-free, albumin-engineered nanoparticle formulation of paclitaxel (American Pharmaceutical Partners, Schaumberg, Illinois), and Taxotere® doxetaxel (Rhone-Poulenc Rorer, Antony, Fran161hlorambucilbucil; Gemzar® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine (Oncovin®); thalidomide (Thalomid®); Navelbine® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluorometlhylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib (Tarceva)) and VEGF-A that reduce cell proliferation and pharmaceutically acceptable salts, acids or derivatives of any of the above.

Further nonlimiting exemplary chemotherapeutic agents that can be administered in methods herein include anti-hormonal agents that act to regulate or inhibit hormone action on cancers such as anti-estrogens and selective estrogen receptor modulators (SERMs), including, for example, tamoxifen (including Nolvadex® tamoxifen), raloxifene, droloxifene, 4-hydroxytamoxifen, trioxifene, keoxifene, LY117018, onapristone, and Fareston® toremifene; aromatase inhibitors that inhibit the enzyme aromatase, which regulates estrogen production in the adrenal glands, such as, for example, 4(5)-imidazoles, aminoglutethimide, Megase® megestrol acetate, Aromasin® exemestane, formestanie, fadrozole, Rivisor® vorozole, Femara® letrozole, and Arimidex® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in aberrant cell proliferation, such as, for example, PKC-alpha, Ralf and H-Ras; ribozymes such as a VEGF expression inhibitor (e.g., Angiozyme® ribozyme) and a HER2 expression inhibitor; vaccines such as gene therapy vaccines, for example, Allovectin® vaccine, Leuvectin® vaccine, and Vaxid® vaccine; Proleukin® rIL-2; Lurtotecan® topoisomerase 1 inhibitor; Abarelix® rmRH; and pharmaceutically acceptable salts, acids or derivatives of any of the above.

In some embodiments, an anti-angiogenesis agent may be administered in combination with an antibody disclosed herein. Non-limiting examples of an anti-angiogenesis agent can include an antibody or other antagonist to an angiogenic agent, e.g., antibodies to VEGF-A (e.g., bevacizumab (Avastin®)) or to the VEGF-A receptor (e.g., KDR receptor or Flt-1 receptor), anti-PDGFR inhibitors such as Gleevec ° (Imatinib Mesylate), small molecules that block VEGF receptor signaling (e.g., PTK787/ZK2284, SU6668, Sutent®/SU11248 (sunitinib malate), AMG706, or those described in, e.g., international patent application WO 2004/113304). Anti-angiogensis agents also include native angiogenesis inhibitors, e.g., angiostatin, endostatin, etc. See, e.g., Klagsbrun and D'Amore (1991) Annu. Rev. Physiol. 53:217-39; Streit and Detmar (2003) Oncogene 22:3172-3179 (e.g., Table 3 listing anti-angiogenic therapy in malignant melanoma); Ferrara & Alitalo (1999) Nature Medicine 5(12):1359-1364; Tonini et al. (2003) Oncogene 22:6549-6556 (e.g., Table 2 listing known anti-angiogenic factors); and, Sato (2003) Int. J. Clin. Oncol. 8:200-206 (e.g., Table 1 listing anti-angiogenic agents used in clinical trials).

In some embodiments, a tumor growth inhibitory agent may be administered in combination with an antibody disclosed herein. Non-limiting examples of growth inhibitory agents include, but are not limited to, agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (Taxotere®, Rhone-Poulenc Rorer), derived from the European yew, is a semisynthetic analogue of paclitaxel (Taxol®, Bristol-Myers Squibb). Paclitaxel and docetaxel promote the assembly of microtubules from tubulin dimers and stabilize microtubules by preventing depolymerization, which results in the inhibition of mitosis in cells.

For treatment of inflammatory or autoimmune or infectious disease conditions or cancer, in some embodiments, an anti-inflammatory drug may be administered in combination with an antibody disclosed herein. The anti-inflammatory drug can be, e.g., a steroid or a non-steroidal anti-inflammatory drug (NSAID). In cases where it is desirable to render aberrantly proliferative cells quiescent in conjunction with or prior to treatment with anti-PAD4 antibodies described herein, hormones and steroids (including synthetic analogs), such as 17a-Ethinylestradiol, Diethylstilbestrol, Testosterone, Prednisone, Fluoxymesterone, Dromostanolone propionate, Testolactone, Megestrolacetate, Methylprednisolone, Methyl-testosterone, Prednisolone, Triamcinolsone, Chlorotrianisene, Hydroxyprogesterone, Aminoglutethimide, Estramustine, Medroxyprogesteroneacetate, Leuprolide, Flutamide, Toremifene, or goserelin (ZOLADEX®), can also be administered to the patient.

An anti-PAD4 antibody described herein can also be combined with a vaccination protocol. Many experimental strategies for vaccination against infectious diseases and tumors have been devised (see Rosenberg, S., 2000, Development of Cancer Vaccines, ASCO Educational Book Spring: 60-62; Logothetis, C, 2000, ASCO Educational Book Spring: 300-302; Khayat, D. 2000, ASCO Educational Book Spring: 414-428; Foon, K. 2000, ASCO Educational Book Spring: 730-738; see also Restifo, N. and Sznol, M., Cancer Vaccines, Ch. 61, pp. 3023-3043 in DeVita et al. (eds.), 1997, Cancer: Principles and Practice of Oncology, Fifth Edition). Thus, in some embodiments, an antibody herein or a vaccine construct encoding an antibody herein may be administered along with an infectious disease or anti-cancer vaccine, for instance, or with a vaccination protocol employing cell-based therapies such as dendritic cells, or vaccine-like particles (VLPs).

In other cases, an antibody herein may be administered in combination with other therapies, such as radiation therapy in the case of a tumor, surgical interventions, or the like.

### EXAMPLES

### Example 1. Generation, Characterization and Humanization of Anti-PAD4 Antibodies

Recombinant human PAD4 protein (rhPAD4) was used to immunize five PAD−/− mice for the purpose of generating antibodies to human PAD4. Four immunization doses of rhPAD4 were administered over 56 days and mouse splenocytes were harvested for hybridoma fusion and culture at day 59-60. Hybridoma fusions were assayed by ELISA for binding to rhPAD4. Cells that tested positively were isolated, cultured, cloned, and stored as hybridoma clones for the production of anti-PAD4 monoclonal antibodies.

For each hybridoma antibody clone, the sequences for the antibody heavy chain and light chain variable domains, VH and VL, and the leader sequences were determined using the Sanger sequencing method and standard bioinformatic methods. The hybridoma antibody clones were assayed again by ELISA for binding to PAD4, and antibodies binding to PAD4 by ELISA were selected for further testing.

### A. Purification and Antibody Characterization Methods

Purification methods. Prior to further analysis, antibodies were purified from 60 mL of cultured supernatant of each hybridoma. Prepacked protein-A columns (GE Healthcare) were used for purification. Each cultured medium containing IgG1 was adjusted to high salt and high pH before passing through the protein-A column. After loading, the column was washed until no detectable protein was found in the flow-through, and eluted with 100 mM phosphate buffer, 25 mM Tris, pH. 2.5. For IgG2 antibodies, supernatant was loaded under neutral pH and eluted with a 1:1 mix of PBS, pH. 7.2 and 100 mM citrate, pH 3.0. Peak fractions of eluate were concentrated, sterile filtered, OD280 measured and stored at −80° C. Concentration was determined based on absorbance: 1 mg/mL of antibody is expected to have an absorbance of about 1.36 in a 1 cm light path cuvette at 280 nm.

Affinity testing methods. Affinity of each antibody clone for a GST-PAD4 fusion protein was determined by Biolayer Interferometry (BLI) using a BLITZ instrument (Forte Bio, Menlo Park, California), using a biosensor chip (Anti-GST chip, Forte Bio, Cat. No. 18-5096). The biosensor was treated with citrate buffer, PBS, or PBS/Ca for 15 seconds; recombinant human GST-PAD4 was added on a drop holder for 80 seconds to prime the biosensor with PAD4; antibody was then added on the drop holder for 80 seconds to allow binding between antibody and the PAD4; PBS/PBS-Ca was added on the drop holder for 60 seconds to remove unbound antibody. The association rate constants, kₒₙ, dissociation rate constants, k_{off}, and equilibrium dissociation constants, K_{D}, were determined using BLITZ Pro Software, version 1.2.0.49.

Activity testing methods. A further assay was conducted to determine the effect of antibody clones on citrulline production by both a recombinant human PAD4 (rhPAD4) and a recombinant human PAD2 (rhPAD2). To determine whether biochemical activity of rhPAD4 and rhPAD2 is inhibited by the antibody clones, 100 nM of each recombinant protein was incubated with increasing concentrations of the antibody clones in 100 mM Tris-HCl, pH 7.6, 1 mM CaCl₂), 2 mM DTT, and 50 mM NaCl for 15 minutes at 37° C. BASE (Nα-benzoyl-L-arginine ethyl ester hydrochloride) substrate (Sigma-Aldrich) (10 mM) was added and the reaction was allowed to proceed for 30 minutes at 37° C. The reaction was quenched with liquid nitrogen and citrulline production was quantified using the COLDER assay (Knipp and Vasak, Anal. Biochem. 2000, 286, 257-2641; Kearney et al. Biochemistry 2005, 44, 10570-10582). To determine the % PAD4 activity, citrulline production was determined in the presence and absence of antibody clone.

B. Antibody Characterization Results

Using the methods described above, twelve of the antibody clones were found to have significant anti-PAD4 activity, as shown in Table 1.

TABLE 1
Antibody Affinity Results.

| Clone # | kₒₙ (1/Ms) | k_{off} (1/s) | K_{D} (M) |
|---|---|---|---|
| 3 | 1.56E+04 | <1.00E−06 | <6.40E−11 |
| 5 | 1.72E+04 | <1.00E−06 | <5.80E−11 |
| 6 | 9.53E+03 | 1.44E−02 | 1.51E−06 |
| 7 | 8.18E+03 | <1.00E−06 | <1.22E−10 |
| 9 | 4.30E+02 | <1.00E−06 | <2.33E−9 |
| 10 | 1.89E+04 | <1.00E−06 | <5.31E−11 |
| 13 | 4.11E+04 | <1.00E−06 | <2.44E−11 |
| 14 | 1.14E+04 | <1.00E−06 | <8.77E−11 |
| 17 | 1.80E+04 | <1.00E−06 | <5.56E−11 |
| 18 | 1.73E+04 | <1.00E−06 | <5.79E−11 |
| 20 | 5.83E+02 | <1.00E−06 | <1.71E−9 |
| 22 | 5.22E+03 | 2.11E−03 | 3.10E−07 |

FIG. 1A shows % PAD4 activity and FIG. 1B shows absolute amounts of citrulline production determined using the activity assay at a concentration of 250 nM antibody. Several antibody clones inhibited the enzymatic activity of PAD4 in the citrulline production assay. In contrast, clone 6 showed a 5.7-fold increase in citrullination by PAD4, indicating that it activated PAD4 activity.

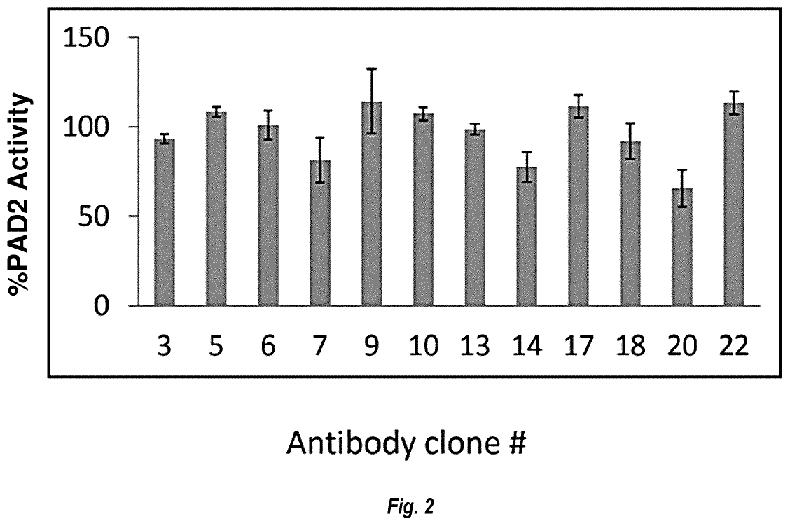

Clones 13 and 20 were found to inhibit PAD4 in a dose-dependent manner (FIG. 1C and FIG. 1D). For clone 13, the VH and VL amino acid sequences, and their CDRs, are shown in FIGS. 1E and 1F, respectively. For clone 20, the VH and VL amino acid sequences, and their CDRs, are shown in FIGS. 1G and 1H. The antibodies did not substantially inhibit the activity of the recombinant human PAD2 as is shown in FIG. 2.

Antibody clones 13 and 20 were humanized and tested as described below.

C. Humanization of Antibody Clones 13 and 20

Antibody clones 13 and 20 were humanized in silico and the resulting humanized antibodies were subsequently made and tested as described herein to create anti-human PAD4 antibodies that are less antigenic in humans and that have characteristics (e.g., affinity, inhibitory activity and other properties and activities described herein) as good or better than those of the parental murine antibodies. Murine amino acid residues were selected for grafting onto human immunoglobulin germline sequences with the aim of retaining or improving the affinity of the murine antibody for human PAD4.

The in silico humanization protocol described here is a general protocol that was used across all murine clones including clones 13 and 20, which is discussed here for the humanization of these two clones. Using the amino acid sequences of the variable domains of clones 13 and 20, the Complementarity Determining Regions (CDRs) of the variable heavy chains (VHs) and variable light chains (VLs) of clones 13 and 20 were identified according to the formulas of Kabat or Chothia (see, e.g., Kabat et al., Sequences of Proteins of Immunological Interest, 5th Ed. Public Health Service, National Institutes of Health, Bethesda, M D (1991); Chothia and Lesk, J. Mol. Biol. 196:901-917 (1987); also see bioinforg.uk/abs/info.html#kabatnum for information on defining the CDR residues according to the Kabat and Chothia formulas and other formulas. The VH and VL sequences of clones 13 and 20 were used to search the V and J human germline databases for heavy chain (HC) and light chain (kappa chain; KC) sequences from the IMGT® human repertoire of immunoglobulin functional genes (imgt.org/genedb/; Giudicelli V., Chaume D. and Lefranc M.-P. Nucleic Acids Res., 33: D256-D261 (2005), Lefranc and Lefranc, Biomedicines, 8(9):319 (2020)). Human germline sequences with closest identities to the murine sequences of clones 13 and 20 were identified. The murine CDR amino acid sequences from clones 13 or 20 were used to replace the corresponding human CDR amino acid sequences within the human germline sequences.

The amino acid sequences of Framework Regions (FRs) of the murine antibody and the corresponding FRs of the human antibody were aligned. This alignment was used to identify murine antibody FR residues that were non-homologous to the corresponding human antibody residues as these were possible candidates for back mutations as discussed below.

Three-dimensional models of clone 13 and 20 antibodies were constructed to determine if, in the course of humanization, mutating a murine amino acid residue at a given position to its human germline analog could be tolerated, specifically whether such amino acid substitution could be expected to exhibit one or more of the following effects: (1) noncovalent binding to antigen; (2) interaction with a CDR; (3) participation in interactions at the VL-VH interface; (4) stabilization of the immunoglobulin structure; or (5) formation of glycosylation sites. Furthermore, it was determined whether the substitution of any amino acid residue in the murine structure with a corresponding amino acid residue in the human antibody FR sequence could disrupt any of those effects. This was determined largely by calculating the free energy change associated with single-point mutations (ΔGₘᵤₜₐₜᵢₒₙ). If deemed likely to be disruptive (ΔGₘᵤₜₐₜᵢₒₙ>0), the original murine amino acid was retained at that position in certain of the humanized sequences, i.e., a “back mutation” of the humanized sequence to the murine sequence at that position was made. It should however be noted that ΔGₘᵤₜₐₜᵢₒₙ, while being a useful measure of the change in overall stability of the protein upon mutagenesis, was often not the only criterion for selecting back mutations. This point is illustrated below with a specific example, in the context of clone 13 humanization. In each case the murine VH and VL chains were humanized separately. For each chain a number of humanized constructs, varying in the framework back mutations but conserved in the CDRs, was designed. VH-VL sequences pairings were given preference based on their frequencies in an internal database of pairings (higher frequency pairings were preferred). This internal database incorporates both public information (based on immunoglobulin structures provided in the Protein Data Bank (www.rcsb.org) and known clinical antibodies) as well as non-public information from prior studies demonstrating favorable expression of certain pairings.

Following the humanization steps in the case of clone 20 resulted in antibodies retaining PAD4 binding affinity, but with high non-specific binding to extracellular matrix (ECM) proteins. Therefore, additional methods and analyses were carried out in the development of those anti-PAD4 antibodies, as described below.

1. Humanization of Clone 13

Upon experimental testing of humanized antibodies of clone 13 that were designed according to the methods described above, it was found that they did not comprise the desired antigen-binding and biophysical characteristics. An inspection of the sequence alignment of the parent antibody with the human germline antibodies nearest in sequence identity to the parent clone provided an important insight, which was that the Gly94 residue adjacent to VH CDR3 (FIG. 1E) was not present in the chosen human germline sequences. Instead, the human germline has a Thr at the equivalent position. Replacing the Gly at position 94 in the murine clone 13 VH parent sequence (SEQ ID NO: 10), which is a highly flexible amino acid residue, with a Thr residue, which is less flexible, likely caused an increased rigidity around the VH CDR3 loop in the resulting humanized construct. Based on energetic considerations (ΔGₘᵤₜₐₜᵢₒₙ) while Thr would have been the preferred residue at that position, keeping Gly at position 94 instead would be much more likely than Thr to maintain the conformational flexibility of the VH CDR3 loop necessary to bind the antigen, a crucial consideration in the design of humanized antibodies. Indeed, the presence of the Thr94 in the CDR graft resulted in loss of activity, as was observed based on experimental testing in several humanized antibodies (data not shown). To remedy this, the Thr was back-mutated to the Gly of the parent sequence. Among others, this extra step in the design of humanized anti-PAD4 antibodies led to antibodies with potent binding to PAD4, despite the relatively low sequence identity of the parent antibody and the selected human germline in the framework regions. Additionally, the resulting humanized antibodies maintained correct secondary structure and had low binding with ECM proteins.

2. Humanization of Clone 20

Humanized antibodies of clone 20, unlike clone 13, showed high affinity binding to the PAD4 as well as high non-specific binding to ECM proteins. An inspection of the VH-VL pairings showed that the human germlines initially chosen for VH and VL humanization were not commonly seen paired together in an antibody. Therefore, a different, and more favorable light chain (LC) germline sequence was chosen from the internal database that allows selection of favorable pairings of VH and VL sequences. This pairing led to humanized clone 20 antibodies that retained high PAD4 affinity and also exhibited reduced binding with ECM proteins compared with the parent clone 20.

3. IgG Germlines for Clones 13 and 20

With the methods described above, human IgG germlines were selected for humanization of the murine VH and VL sequences (See the Sequence Table, which indicates the germlines used in the “Description” column).

Two pairs of human variable germlines (VH germlines IGHV1-46*01 paired with VL germline IGKV4-1*01 and VH germline IGHV1-18*01 paired with VL germline IGKV1-39*01) were deemed equally suitable for humanizing clone 20, and accordingly humanized constructs involving both pairs of germlines for clone 20 were made.

For both clones 13 and 20, besides the CDR-grafted humanized constructs, constructs with certain back mutations were made in view of structural considerations. Clone 13-based antibodies are denoted hz13-1 to hz13-12 herein, while the murine anti-human clone 13 antibody is denoted mAb13. Similarly, clone 20-based antibodies are denoted hz20-1 to hz20-14 while the murine anti-human antibody is denoted mAb20. Properties of humanized clone 13 and clone 20 antibodies are described in the examples that follow.

### Example 2: Expression and Purification of Humanized Antibodies

Expression methods. The coding sequence for PAD4 mAb heavy chain (HC) and light chain (LC) were cloned into pTT22 gate or pTT5 vector. The constructs were amplified and used to transiently transfect Expi293F cells using the Expi293 Expression System (Thermo Fisher Scientific, catalog #A14635). 0.5 mg DNA was used to transfect each liter of cell culture. First, to check for expression levels, the PAD4 monoclonal antibodies were screened at a 3-ml scale with HC:LC ratios of 1:1 and 1:2. Samples were harvested on day 5 after transient transfection and their titers were analyzed using the Octet® system (Sartorius).

For production of each antibody at a 1-L scale, 900 ml of Expi293F cells were seeded in Expi293 Expression medium at 2.8×10e6 cell/mL using a 2-L corning flask. 0.5 mg DNA/L culture was added to 50 ml of prewarmed OPTIMEM media and mixed gently.

Ratios of HC:LC used were either 1:1 or 1:2, depending on preliminary screening results. ExpiFectamine™ 293 was mixed by pipetting gently up and down prior to use. 1.35 mL of Expifectamine293 was added to 50 mL of prewarmed OPTIMEM media and mixed gently. The mixture was incubated for 5 minutes. A DNA:ExpiFectamine™ 293 ratio of 1:2.7 was used. The diluted ExpiFectamine™ 293 reagent was added to the diluted DNA and mixed by swirling to produce ExpiFectamine™ 293-DNA complexes. The complexes were incubated at room temperature for 20 minutes. Then, 100 mL of transfection mixture was added to a shaker flask containing the 900 mL of cells and the flask was gently swirled during the addition. The cells were incubated at 37° C. and 8% CO₂ with shaking at 125 rpm in humidified atmosphere. Sixteen to twenty hours post-transfection), 5.0 mL of ExpiFectamine™ 293 Transfection Enhancer 1 and 50 mL of ExpiFectamine™ 293 Transfection Enhancer 2 was added to each flask. Antibodies were harvested on day 5 by centrifuging the samples at 2000 rpm. The supernatant for each sample was saved and filtered through a 0.2-μm filter in preparation for antibody purification.

Purification methods. Harvested PAD4 mAb supernatants from HEK 293 expression system was first captured using rProtein A Sepharose FF (Cytiva) affinity column pre-equilibrated with DPBS (Corning) on the AKTA Pure25 system. After capturing, column was washed with DPBS until reached baseline then PAD4 mAb was eluted with 80 mM NaAc (pH 2.8) into the collection bottle containing approximately 1/10V (of total elution volume) 1M Tris-HCl (pH 8.0) to neutralize the PAD4 mAb immediately upon elution. This rProtein A elution pool of PAD4 mAb was concentrated down to a smaller volume and further polished by SEC on a 26/600 Superdex-200 to remove any aggregates or multimers. Final 5200 PAD4 mAb monomeric peak was pooled, endotoxin was lowered using Mustang-Q Syringe filter (PALL Corp) and filter sterilized using 0.2 μm filter.

### Example 3: Binding Affinity of the Humanized Clone 13 and Humanized Clone 20 Antibodies

Humanized constructs of clone 13 and 20 were formatted as IgG1.3 antibodies. Their binding to human PAD4 was measured by Surface Plasmon Resonance (SPR). SPR measurements were performed with a Biacore® 8K, 8K+, or T200 instrument (Cytiva). The association rate constants, kₒₙ, dissociation rate constants, k_{off}, and equilibrium dissociation constants, K_{D}, of the antibodies with PAD4 were determined from these SPR measurements.

For SPR measurements, either an anti-human capture surface or Protein A surface (Cytiva catalog #29127555) was used. Anti-human capture surface was prepared by immobilizing anti-human capture antibody (Cytiva catalog #29234600) onto flow cells of a CM5 or CM4 biosensor following the manufacturer's amine coupling protocol (Cytiva catalog #BR-1006-33). SPR experiments were conducted at 37° C. using HBS-P (150 mM NaCl, 10 mM HEPES, pH 7.6, 0.05% Tween-20) (TEKNOVA catalog #H8032) with additional 150 mM NaCl and 1 mM or 2 mM CaCl₂) (see Table 2) or 2 mM EDTA (see Table 3) as running buffer. Antibodies were diluted to 1.5 μg/mL in the running buffer, and were captured across active biosensor flow cells at 10 μL/min for 15 to 30 seconds. Several concentrations of PAD4 (for instance, from 0.59 nM to 75 nM or from 1.56 nM to 50 nM for clone 13 and its derivatives and from 2.34 nM to 300 nM for clone 20 and its derivatives), were prepared using the running buffer, and injected over the captured antibodies at 30 μL/min. The association and dissociation of the antibodies with PAD4 were measured. One 30 second injection followed by one 15 second injection of 10 mM glycine pH 1.5 was used to regenerate the Protein A capture surface between assay cycles. For anti-human capture surface, two 30 second injections of 3 M MgCl₂ was used for regeneration. SPR measurements for mAb20 and mAb 13 SPR were done in the presence of 2 mM CaCl₂). The remaining antibodies were tested in the presence of 1 mM CaCl₂).

Rate constants kₒₙ and k_{off} were derived from reference flow cell- and 0 nM blank-subtracted sensorgrams, and were fit to a 1:1 binding model in Biacore® Insight Evaluation software version 3.0.12.15655. Deviations from 1:1 binding model were observed, which may be caused by increased dimerization of PAD4 at higher concentrations. The equilibrium dissociation constant, K_{D}, was calculated as the ratio of rate constants k_{off}/kₒₙ for each PAD4 antibody. The kₒₙ, k_{off}, and K_{D} values for the antibodies are presented in Table 2 and Table 3. For those antibodies that were tested in multiple SPR experiments, the average value and standard deviation from two or three separate experiments are listed in Tables 2 and 3.

TABLE 2
Binding Affinity of Humanized Antibodies as Determined
by SPR at 37° C., in the presence of Ca²⁺

| Antibody | kₒₙ (M⁻¹ s⁻¹) | k_{off} (s⁻¹) | K_{D} (nM) |
|---|---|---|---|
| mAb20* | 4.3E+04 ± 1.1E+04 | 6.2E−03 ± 5.7E−04 | 150 ± 50** |
| hz20-1 | 5.5E+05 ± 5.8E+05 | 8.9E−04 ± 2.2E−04 | 2.7 ± 1.5 |
| hz20-2 | 2.9E+05 ± 5.9E+04 | 2.9E−04 ± 1.8E−04 | 1.1 ± 0.9 |
| hz20-3 | 5.0E+05 | 2.3E−04 | 0.5 |
| hz20-4 | 2.7E+05 ± 1.5E+04 | 4.6E−04 ± 2.3E−04 | 1.7 ± 0.8 |
| hz20-5 | 2.5E+05 ± 4.5E+04 | 4.5E−04 ± 1.5E−04 | 1.9 ± 0.9 |
| hz20-6 | 3.9E+05 | 2.5E−04 | 0.6 |
| hz20-7 | 3.5E+05 | 2.6E−04 | 0.8 |
| hz20-8 | 3.2E+05 ± 5.2E+04 | 2.5E−04 ± 1.9E−04 | 0.8 ± 0.7 |
| hz20-9 | 4.9E+05 | 1.5E−04 | 0.3 |
| hz20-10 | 6.4E+05 | 1.7E−04 | 0.3 |
| hz20-11 | 3.0E+05 ± 3.2E+04 | 2.5E−04 ± 1.7E−04 | 0.9 ± 0.7 |
| hz20-12 | 3.2E+05 ± 4.8E+04 | 1.9E−04 ± 1.0E−04 | 0.6 ± 0.4 |
| hz20-13 | 3.9E+05 | 1.9E−04 | 0.5 |
| hz20-14 | 3.4E+05 | 2.0E−04 | 0.6 |
| mAb13* | 3.5E+05 ± 7.1E+03 | 1.1E−03 ± 2.7E−04 | 3.2 ± 0.8 |
| hz13-1 | 8.8E+05 ± 2.3E+05 | 1.5E−04 ± 8.6E−05 | 0.2 ± 0.1 |
| hz13-2 | 1.7E+06 ± 3.6E+05 | 1.2E−04 ± 5.7E−05 | 0.1 ± 0.03 |
| hz13-3 | 1.5E+06 ± 1.8E+05 | 1.3E−04 ± 6.9E−05 | 0.1 ± 0.05 |
| hz13-4 | 9.3E+05 | 1.3E−04 | 0.1 |
| hz13-5 | 1.6E+06 ± 7.6E+04 | 1.2E−04 ± 5.8E−05 | 0.1 ± 0.04 |
| hz13-5 D31E | 1.32E+06 ± 9.12E+05 | 1.71E−04 ± 7.56E−05 | 0.147 ± 0.057 |
| hz13-6 | 1.6E+06 ± 2.8E+05 | 1.2E−04 ± 3.6E−05 | 0.1 ± 0.02 |
| hz13-7 | 1.2E+06 ± 1.2E+05 | 1.5E−04 ± 5.1E−05 | 0.1 ± 0.05 |
| hz13-8 | 1.7E+06 ± 2.9E+05 | 1.4E−04 ± 4.4E−05 | 0.1 ± 0.02 |
| hz13-9 | 2.0E+06 ± 7.4E+05 | 1.6E−04 ± 3.8E−05 | 0.1 ± 0.01 |
| hz13-10 | 2.2E+06 | 1.7E−04 | 0.1 |
| hz13-11 | 1.9E+06 | 1.5E−04 | 0.1 |
| hz13-12 | 3.1E+06 | 2.0E−04 | 0.1 |
| hz13-12 D31E | 2.7E+06 | 3.0E−04 | 0.1 |

*SPR at 2 mM CaCl₂; all the humanized antibodies were tested in the presence of 1 mM CaC12
**Biphasic binding response observed, which yields an approximate KD value

The results indicate that the antibodies bind PAD4 with similar affinities, whether calcium is present (Table 2) or absent (due to binding by EDTA) (Table 3).

TABLE 3
Binding Affinity of Humanized Antibodies as Determined
by SPR at 37° C., in the presence of EDTA

| Anti- | | | |
|---|---|---|---|
| body | kₒₙ (M⁻¹ s⁻¹) | k_{off} (s⁻¹) | K_{D} (nM) |
| hz20-2 | 3.1E+05 ± 8.1E+04 | 3.2E−04 ± 1.1E−04 | 1.0 ± 0.1 |
| hz20-3 | 3.4E+05 ± 4.5E+03 | 3.1E−04 ± 2.0E−04 | 0.9 ± 0.6 |
| hz20-4 | 2.4E+05 ± 2.9E+04 | 7.3E−04 ± 5.3E−04 | 3.0 ± 1.9 |
| hz20-6 | 3.0E+05 ± 1.7E+04 | 3.7E−04 ± 2.2E−04 | 1.2 ± 0.7 |
| hz20-5 | 2.4E+05 ± 7.3E+04 | 4.0E−04 ± 4.8E−05 | 1.7 ± 0.3 |
| hz20-7 | 3.8E+05 ± 6.8E+04 | 2.1E−04 ± 6.4E−05 | 0.5 ± 0.1 |
| hz20-8 | 3.6E+05 ± 9.5E+04 | 2.2E−04 ± 3.5E−05 | 0.6 ± 0.1 |
| hz20-9 | 4.8E+05 ± 1.1E+05 | 1.8E−04 ± 4.2E−05 | 0.4 ± 0.0 |
| hz20-10 | 4.8E+05 ± 1.5E+05 | 1.8E−04 ± 5.2E−05 | 0.4 ± 0.0 |
| hz20-11 | 3.9E+05 ± 1.3E+05 | 2.2E−04 ± 1.4E−05 | 0.6 ± 0.2 |
| hz20-12 | 3.8E+05 ± 1.1E+05 | 2.1E−04 ± 2.5E−05 | 0.6 ± 0.1 |
| hz20-13 | 4.4E+05 ± 1.7E+05 | 1.8E−04 ± 2.5E−05 | 0.4 ± 0.1 |
| hz20-14 | 4.5E+05 ± 1.8E+05 | 1.8E−04 ± 2.2E−05 | 0.4 ± 0.1 |
| hz20-1 | 2.2E+05 ± 8.2E+04 | 5.4E−04 ± 5.1E−05 | 2.6 ± 0.7 |
| hz13-1 | 1.3E+06 | 4.0E−04 | 0.3 |
| hz13-2 | 2.0E+06 | 2.5E−04 | 0.1 |
| hz13-3 | 1.9E+06 | 2.1E−04 | 0.1 |
| hz13-4 | 1.4E+06 | 3.2E−04 | 0.2 |
| hz13-5 | 2.1E+06 | 2.1E−04 | 0.1 |
| hz13-5 | 1.17E+06 ± 1.16E+04 | 3.58E−04 ± 1.44E−05 | 0.306 ± 0.015 |
| D31E | | | |
| hz13-6 | 1.7E+06 | 2.3E−04 | 0.1 |
| hz13-7 | 2.0E+06 | 2.5E−04 | 0.1 |
| hz13-8 | 1.9E+06 | 1.6E−04 | 0.1 |
| hz13-9 | 2.0E+06 | 1.9E−04 | 0.1 |

### Example 4: Enzyme Kinetics of the Humanized Antibodies

Enzymatic Blocking Assay. The antibodies were tested for inhibition of PAD4 activity against the substrate TSTGGRQGSHH (SEQ ID NO: 216). PAD4 converts the arginine in the peptide substrate TSTGGRQGSHH to citrulline. This reaction can be monitored via RapidFire™ mass spectroscopy (Agilent). Antibodies were tested using TSTGGRQGSHH to determine if they were able to inhibit the activity of PAD4, resulting in a decrease in the citrulline product formation. The assay buffer was as follows: 100 mM HEPES pH 7.4, 200 mM NaCl, 2 mM CaCl₂, and 5 mM DTT. The assay conditions were as follows: 35 nM recombinant human PAD4, 500 μM TSTGGRQGSHH peptide, and 2 μl antibody solution. The total volume for each reaction in this assay was 20 μL. The stop solution used was 10% formic acid.

The antibodies were serially diluted at 3-fold intervals in the assay buffer. The highest antibody concentration was at least 10-times less concentrated than the stock concentration used in dose response curves.

Reaction mixtures were prepared in a microtiter plate using the assay buffer and conditions described above. 2 μl of each antibody solution was used in each well. The reaction mixtures were incubated at room temperature for 30 minutes. To stop the reaction, 10 μl of each of the reaction mixtures was then mixed with 40 μl of 10% formic acid. Before RapidFire™ mass spectroscopy analysis, the plate was stored at −80° C.

Thawed samples were loaded onto the Agilent RapidFire™ 300 and an Agilent “C” (C18) cartridge using a mobile phase of water containing 0.09% formic acid/0.01% trifluoroacetic acid for 3000 ms of desalting flowing at a rate of 1.5 ml/min. Once the samples were loaded and washed, a mobile phase of acetonitrile containing 0.09% formic acid/0.01% trifluoroacetic acid was used to elute the samples directly onto a Sciex API 4000 triple quadrupole mass spectrometer for 3000 ms at a flow rate of 1.25 ml/min. MRM transitions for substrate and product were monitored in positive ESI mode at m/z=562.3/969.7 and m/z=562.8/541.3 respectively. The dwell time for each transition was set at 100 ms, and the ESI voltage was used at 5500 with a source temperature of 650° C. Extracted ion peaks for each transition were integrated using the RapidFire™ Integrator software. These values were used to calculate percent inhibition where, Percent Inhibition=(1−Sample Reaction/Control Reaction)*100. The data were fit using a four-parameter logistic Hill equation in GraphPad® Prism™ to determine IC50. The resulting IC50 data are shown in Table 4.

TABLE 4
Kinetic Data for Humanized Antibodies.

| Antibody | IC₅₀ (average) (in nM) |
|---|---|
| mAb20 | 104 ± 30 |
| hz20-1 | 77 |
| hz20-2 | 73 ± 47 |
| hz20-3 | 58 ± 10 |
| hz20-4 | 118 |
| hz20-5 | 56.5 |
| hz20-6 | 64 ± 46 |
| hz20-7 | 64.7 |
| hz20-8 | 71.9 |
| hz20-9 | 107 |
| hz20-10 | 57.4 |
| hz20-11 | 65.7 |
| hz20-12 | 59 |
| hz20-13 | 52.6 |
| hz20-14 | 56.9 |
| mAb13 | 30 ± 9 |
| hz13-1 | 125 |
| hz13-2 | 96.4 |
| hz13-3 | 23.3 |
| hz13-4 | 115 |
| hz13-5 | 48.2 |
| hz13-6 | 39.1 |
| hz13-7 | 170 |
| hz13-8 | 62.8 |
| hz13-9 | 76 |
| hz13-10 | 55.8 |
| hz13-11 | 88.7 |
| hz13-12 | 42.4 |

### Example 5: Extracellular Matrix (ECM) Scores for the Humanized Antibodies

To determine whether the humanized antibodies might bind nonspecifically to extracellular matrix proteins, the following test was performed. 96-well Corning Thin-Layer Matrigel Matrix pre-coated extracellular matrix (ECM) plates were incubated for one hour at room temperature with 300 μL of blocking buffer (10% FCS in TBS, Alfa Aesar catalog #J61327). After incubation, antibodies at 1, 0.2, and 0.04 μM in 100 μL of fresh blocking buffer were added to the wells. Six wells had no sample addition for background and ECM score calculations. After one hour of sample incubation, the samples were removed and plates were washed with PBS-T wash buffer 3 times. 10 ng/mL of goat anti-human IgG-HRP conjugated detection antibody (Jackson ImmunoResearch catalog #109-035-008) was added at 10011.1 to each well. After another hour incubation at room temperature, the wells were washed 3 times with PBS-T wash buffer. After washing, 100 μl of TMB substrate (Surmodics catalog #TMBW-1000-01) was added to each well and allowed to react for 15 minutes, followed by addition of 100 μl of 1 M phosphoric acid stop solution. Absorbance was then read on a microplate reader at 450 nm and referenced at 620 nm. The ECM scores presented in Table 5 were calculated by dividing the absorbance values of the sample wells by the absorbance of the wells with no sample addition. A lower ECM score is generally desirable because it indicates less binding to extracellular matrix proteins. All humanized clone 13 constructs showed low ECM scores, whereas about half of the humanized clone 20 constructs showed higher ECM scores (a level of 6 or above; this cut off was chosen since all of the 48 clinically approved monoclonal antibodies that were tested using the same protocol showed ECM scores less than 6).

TABLE 5
ECM Scores for Humanized Antibodies.

| Antibody | ECM Score (1 μM) |
|---|---|
| hz20-1 | 1 |
| hz20-2 | 6 |
| hz20-3 | 20 |
| hz20-4 | 1 |
| hz20-5 | 3 |
| hz20-6 | 16 |
| hz20-7 | 5 |
| hz20-8 | 4 |
| hz20-9 | 19 |
| hz20-10 | 18 |
| hz20-11 | 4 |
| hz20-12 | 3 |
| hz20-13 | 19 |
| hz20-14 | 20 |
| hz13-1 | 1 |
| hz13-2 | 1 |
| hz13-3 | 1 |
| hz13-4 | 1 |
| hz13-5 | 1 |
| hz13-6 | 1 |
| hz13-7 | 1 |
| hz13-8 | 2 |
| hz13-9 | 2 |
| hz13-10 | 1 |
| hz13-11 | 1 |
| hz13-12 | 1 |

### Example 6: Antibody Hydrophobicity

Surface hydrophobicity of each humanized antibody was assessed by hydrophobic interaction chromatography (HIC). Ten μg of each antibody sample was loaded on a TSKgel Butyl-NPR column (4.6 mm×3.5 cm, 2.5 μm particle size, Tosoh P/N 14947) on an Agilent 1260 Infinity II HPLC system. A linear gradient of mobile phase A (0.1 M sodium phosphate pH 7.0, 2 M ammonium sulfate) and mobile phase B (solution 0.1 M sodium phosphate pH 7.0) was used for 20 min at a flow rate of 1.0 ml/min at 25° C. column temperature. HIC retention times (RT) of the humanized antibodies are listed in Table 6. Based on the HIC results, all the humanized antibodies have acceptable surface hydrophobicity when a set of 48 clinically approved monoclonal antibodies was used as a reference. (Approximately 75% of these clinically approved monoclonal antibodies, which were tested using the same experimental protocol, showed RT lower than 11 minutes).

TABLE 6
HIC Data for Humanized Antibodies.

| Antibody | HIC RT (minute) |
|---|---|
| hz20-1 | 10.2 |
| hz20-2 | 10 |
| hz20-3 | 9.7 |
| hz20-4 | 10 |
| hz20-5 | 10.2 |
| hz20-6 | 9.8 |
| hz20-7 | 10.2 |
| hz20-8 | 10.2 |
| hz20-9 | 10 |
| hz20-10 | 10 |
| hz20-11 | 10.3 |
| hz20-12 | 10.2 |
| hz20-13 | 10 |
| hz20-14 | 10 |
| hz13-1 | 9.6 |
| hz13-2 | 9.8 |
| hz13-3 | 9.8 |
| hz13-4 | 9.7 |
| hz13-5 | 9.8 |
| hz13-5 D31E | 9.9 |
| hz13-6 | 9.8 |
| hz13-7 | 9.7 |
| hz13-8 | 9.8 |
| hz13-9 | 9.8 |
| hz13-10 | 9.6 |
| hz13-11 | 9.6 |
| hz13-12 | 9.8 |

### Example 7: Stability of Humanized Antibodies

In experiments described in this Example, the stability of humanized clone 13 and humanized clone 20 antibodies (each with sequences as disclosed herein, including an IgG1.3 constant region, expressed and purified as described in Example 2) was assessed.

A. Freeze/Thaw Stability

The freezing stability of the antibodies was determined by performing 5 freeze/thaw cycles. A concentration of 1 mg/mL and 500 μL/vial of each antibody was added to a 2 mL screw cap clear vial (Agilent). The vials were placed in a Biocision CoolCell™ and stored at −80° C. for 2 hours. The CoolCell™ was removed and placed at room temperature (˜23° C.) for 2 hours. This was repeated a total of 5 times and the samples were analyzed. The antibodies subjected to freeze/thaw cycles did not show any significant change compared with the initial starting samples (data not shown).

B. Thermal Stability as Assessed by Melting Temperature, Aggregation Temperature, and Hydrodynamic Volume

1. Methods

The UNcle® biologics stability screening platform (Unchained Labs, Pleasanton, CA) was used to determine the melting temperature (Tₘ) using intrinsic fluorescence and aggregation temperature (T_{agg}) values using static light scattering, and for performing dynamic light scattering (DLS) pre- and post-thermal melt using fluorescence. The antibodies were loaded at 1 mg/mL in (i) 20 mM Histidine, 260 mM Sucrose, 50 μM DTPA, 0.05% PS80 for the pH 6.0 condition and in (ii) 20 mM Tris, 260 mM Sucrose, 50 μM DTPA, 0.05% PS80 for the pH 8.3 condition. DLS was performed on the samples at 20° C. with an acquisition time of 5 seconds and a total of 4 acquisitions. DLS was again performed post thermal ramp at a temperature of 90° C. A stepped thermal ramp was performed to determine the Tₘ and T_{agg} values starting at 20° C. and ending at 90° C. with a ramping rate of 1° C./minute with a 30 second step hold time. Data analysis was performed using the UNcle® analysis software. The Tₘ values were determined using the differential values between temperature steps. The T_{agg} values were determined using static light scattering (SLS) at 266 nm.

2. Results

The melting temperature Tₘ was determined for humanized PAD4 antibodies to assess their thermal stability. The Tₘ and T_{agg} values were determined at pH 6.0 and pH 8.3 as described above and the results are shown in Table 7. Overall, the antibodies showed higher thermal stability at pH 6.0 than pH 8.3, except that hz13-1 and hz13-7 had equivalent stability at pH 6.0 and pH 8.3. The Tₘ values at pH 6.0 ranged from −64° C. to −69° C., which are typical for an antibody. The Tₘ values at pH 8.3 ranged from −58° C. to −67° C., indicating that the antibodies typically had lower thermal stability in the higher pH buffer. Likewise, the antibodies have higher T_{agg} values at pH 6.0 (64° C. to 76° C.) compared to those at pH 8.3 (61° C. to 69° C.). Dynamic light scattering (DLS) was performed at the start of the thermal ramp and showed at hydrodynamic diameter of ˜10 nm for all constructs (data not shown), which is typical for an antibody.

TABLE 7
Melting temperature and Aggregation Temperature results in ° C.

| | |
|---|---|
| Tm values | Tagg values |

| Antibody | pH 6.0 | pH 8.3 | pH 6.0 | pH 8.3 |
|---|---|---|---|---|
| hz20-1 | 64.10 | 60.98 | 64.22 | 61.4 |
| hz20-2 | 67.30 | 61.67 | 69.44 | 63.6 |
| hz20-3 | 66.90 | 61.50 | 69.89 | 63.6 |
| hz20-4 | 65.65 | 62.51 | 66.94 | 64.6 |
| hz20-5 | 65.65 | 62.96 | 70.46 | 64.9 |
| hz20-6 | 66.55 | 61.69 | 71.05 | 63.7 |
| hz13-1 | 67.30 | 66.94 | 76.12 | 69.3 |
| hz13-2 | 68.65 | 65.91 | 74.20 | 66.8 |
| hz13-3 | 68.90 | 64.55 | 72.13 | 64.8 |
| hz13-4 | 68.95 | 63.20 | 70.35 | 64 |
| hz13-5 | 65.70 | 59.85 | 66.61 | 61.6 |
| hz13-6 | 63.95 | 57.87 | 64.72 | 59.6 |
| hz13-7 | 66.70 | 66.85 | 72.87 | 66.2 |
| hz13-8 | 68.43 | 64.23 | 70.83 | 65.2 |
| hz13-9 | 68.37 | 63.13 | 69.98 | 64.3 |
| hz13-10 | 67.90 | 63.60 | 70.12 | 65.94 |
| hz13-11 | 66.30 | 61.10 | 68.69 | 63.63 |
| hz13-12 | 66.90 | 61.40 | 67.97 | 64.70 |
| hz20-7 | 67.40 | 62.50 | 70.35 | 64.52 |
| hz20-8 | 66.80 | 63.60 | 69.65 | 65.61 |

C. Chemical Stability as Assessed by Size Exclusion Chromatography (SEC) and Peptide Mapping Volume

3. Methods

Antibody Handling. The antibodies were stored under temperature-controlled conditions at 4° C. or 40° C. in a Binder incubator. A concentration of 1 mg/mL with a formulation of 20 mM Histidine, 260 mM Sucrose, 50 μM DTPA, 0.05% PS80 for pH 6.0 with 1 mL/vial of each antibody was added to a 2 mL screw cap clear vial (Agilent) and parafilm tape was secured around the top to insure a tight seal for the initial for the truncated stability study. The vials were stored in the appropriate incubator. 500 μL of the sample was removed from each vial at 2 and 4 weeks for analysis. Chemical liabilities were analyzed by size exclusion chromatography and peptide mapping.

Size Exclusion Chromatography (SEC). SEC was used to determine the size homogeneity of the antibodies after 2 weeks and after 4 weeks of storage. An Agilent 1260 Infinity™ system was used with an Advancebio™ SEC 300 Å, 4.6×300 mm, 2.7 μm column. The running buffer was 100 mM potassium phosphate, 250 mM sodium chloride, pH 6.8. An amount of 25 μg of the sample was injected onto the column and run at a rate of 0.5 mL/minute for 15 minutes. The chromatograms were analyzed at 280 nm and the area of the peaks was used to determine the percentages of monomer, high molecular weight species, and low molecular weight species. A gel filtration standard from Bio-Rad Laboratories was used before and after runs to ensure column integrity.

LC-MS Tryptic Peptide Mapping and Analysis. The antibodies were denatured in the presence of 0.2% Rapigest surfactant (Waters Corp.) and reduced by dithiothreitol (DTT) at 80° C. for 30 min, alkylated by iodoacetamide (IAM) at room temp for 30 min in dark, and digested by trypsin at 37° C. for 4 hrs in Tris pH 7.4 followed by acidic quench.

Peptides were analyzed on an ACQUITY UPLC system (Waters, Manchester, U.K.) coupled to a Q-Exactive™ Plus mass spectrometer (Thermo Scientific, San Jose, CA). Peptides were eluted from a Waters BEH C18 column (130A 1.7 μm 2.1×150 mm, product #186002353), heated at 50° C., using a 60 min LC gradient. The gradient setting was 0.2% to 30% solvent B in 46 min at 0.2 mL/min flow rate. Solvent A was 0.1% formic acid in water and solvent B was 0.1% formic acid in acetonitrile. Mass spectrometer was operated under positive ion mode with ESI voltage at 3.5 kV, capillary temperature: 250° C., scan range: 320-1800, sheath gas flow rate: 45.

Peptide mapping data was analyzed by Biologic™ software (Protein Metrics, Cupertino, CA) with precursor mass tolerance of 6 ppm, and fragment mass tolerance of 10 ppm. Carbamidomethylation was set as the fixed modification, and variable modifications were searched against oxidation and deamidation. The MS/MS spectra and the levels of modification were manually verified and calculated. The oxidation, deamidation and isomerization levels of peptides were monitored. % Relative Modification was calculated based on AUC of modified peptide/(AUC of modified peptide+AUC of native peptide)*100.

4. Results

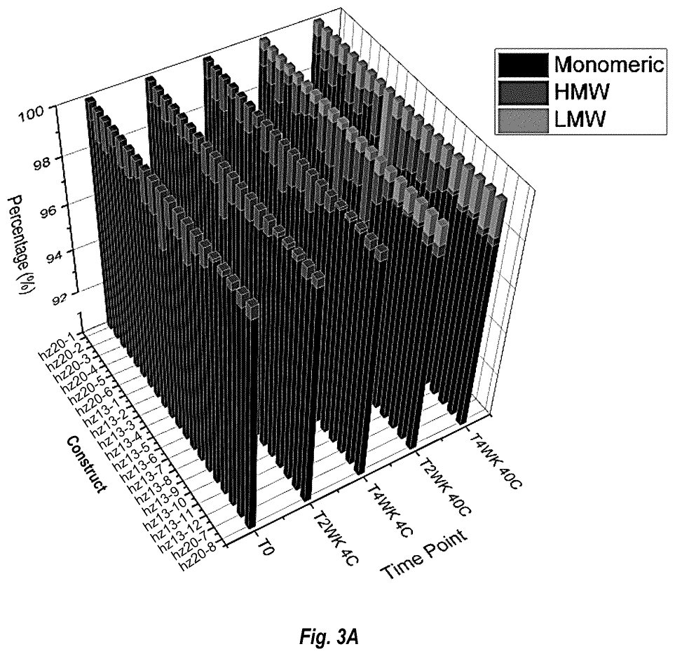

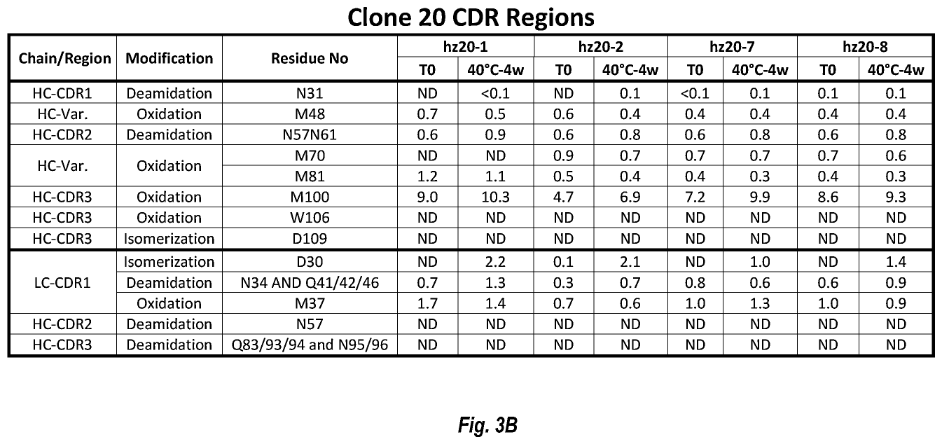

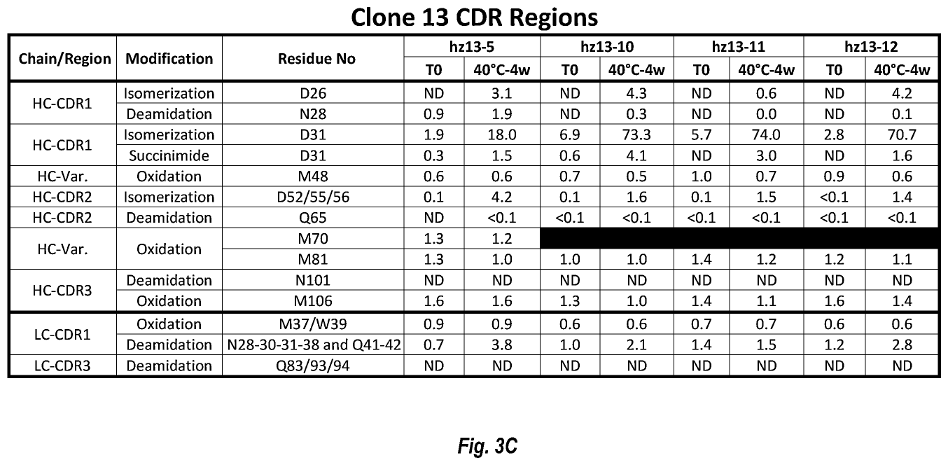

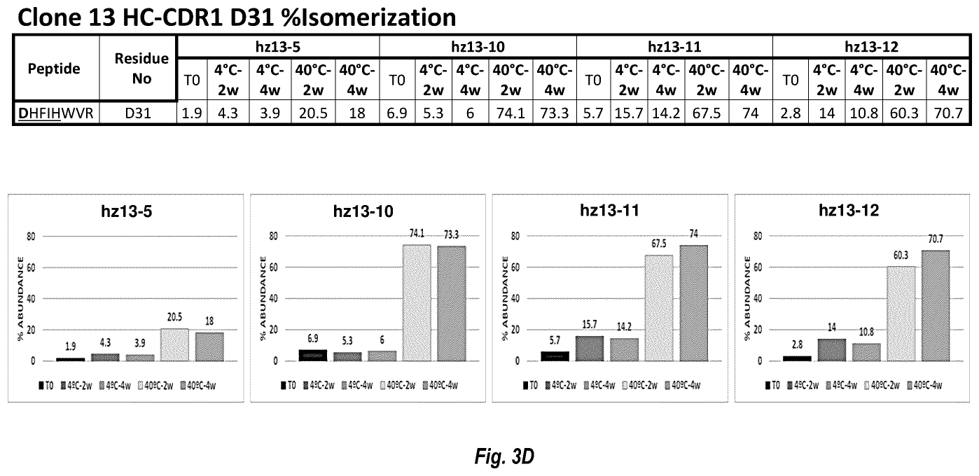

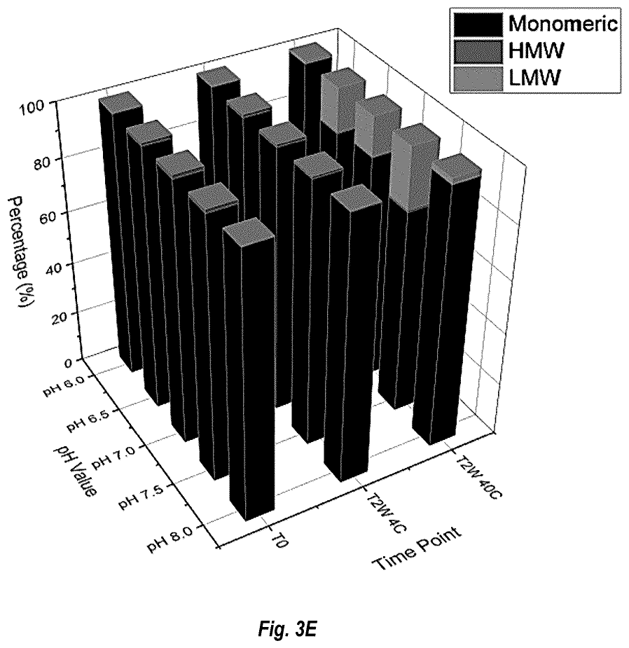

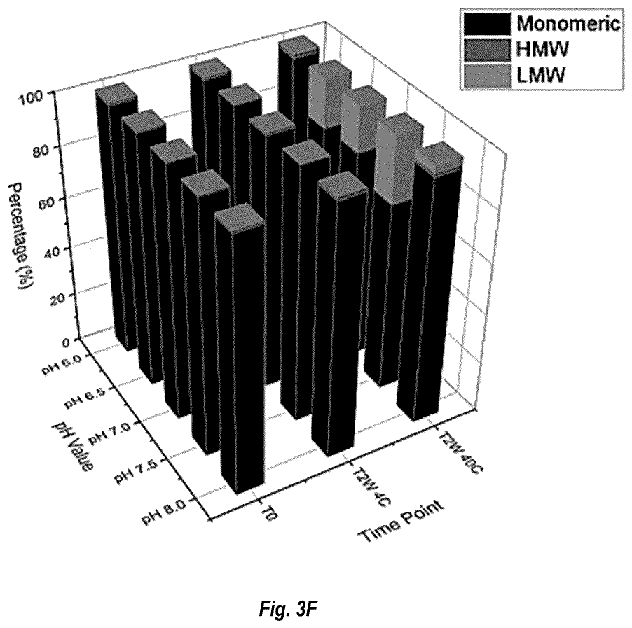

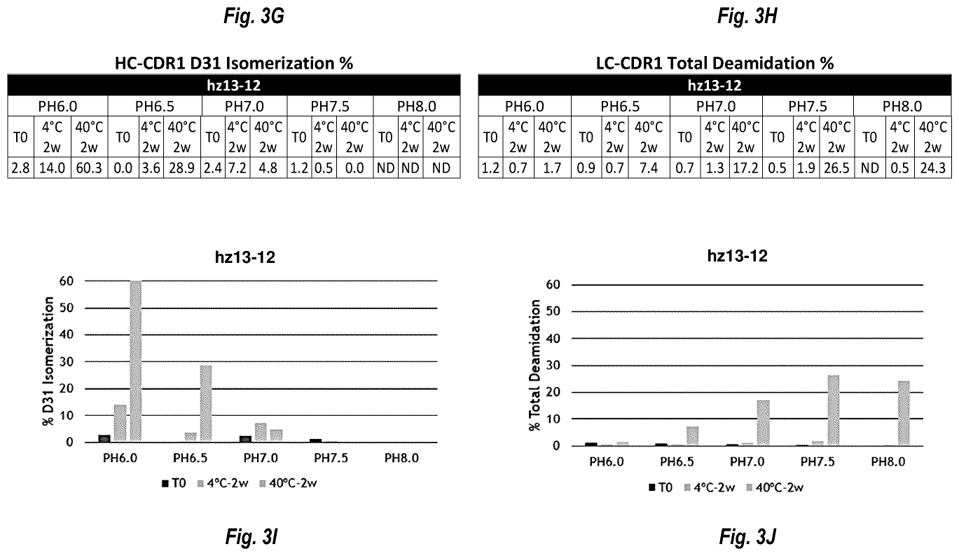

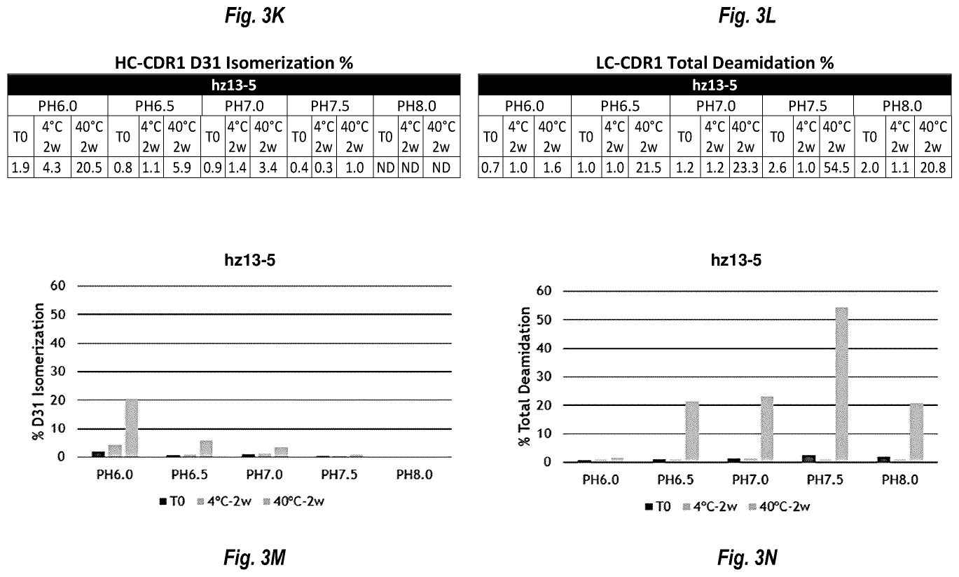

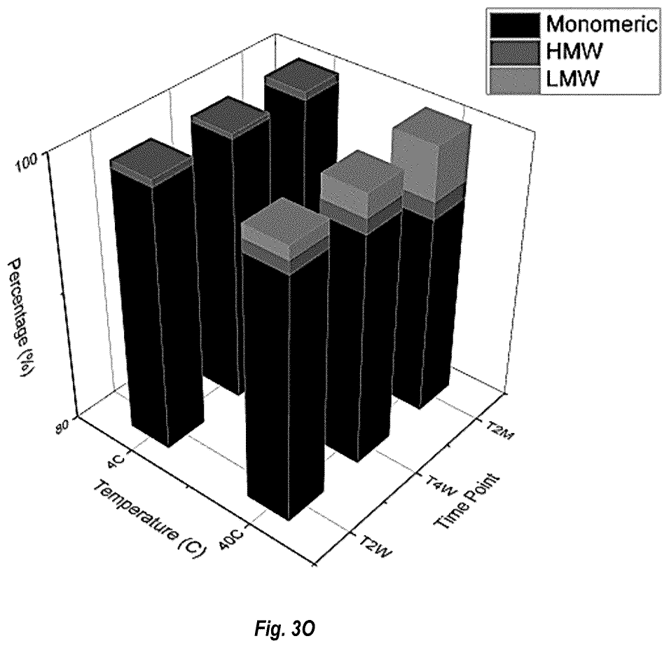

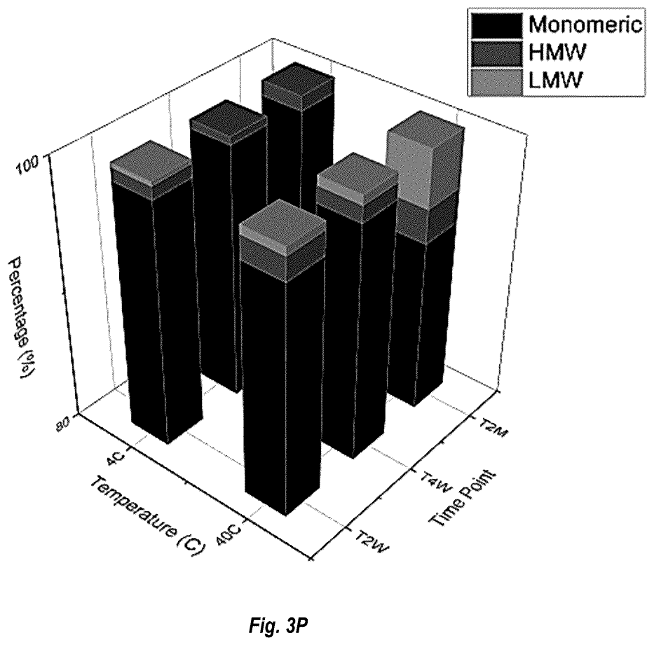

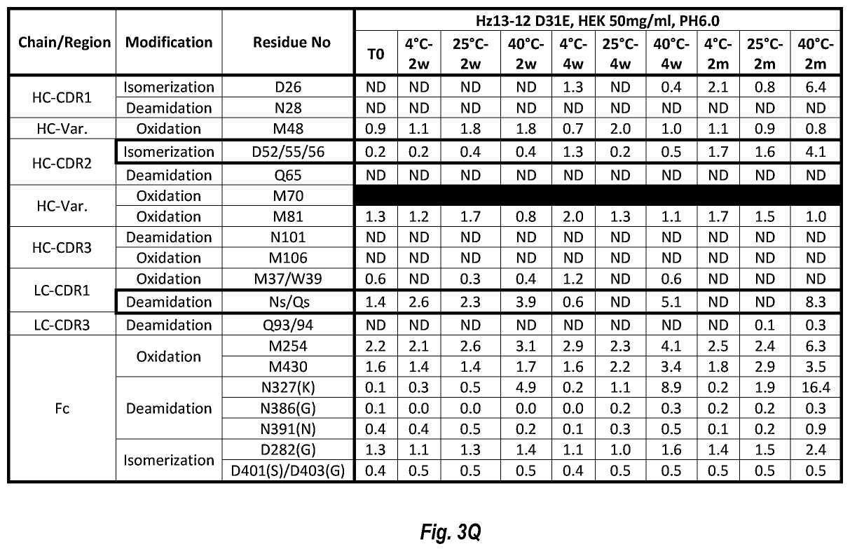

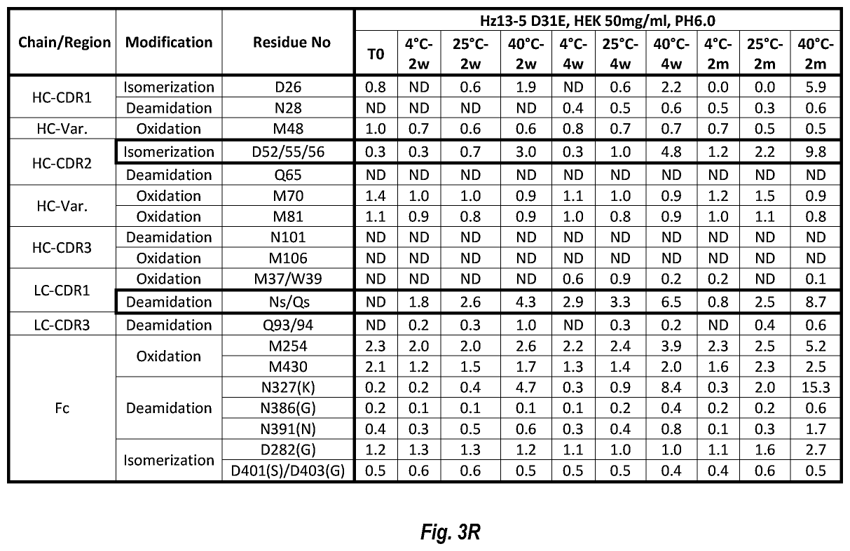

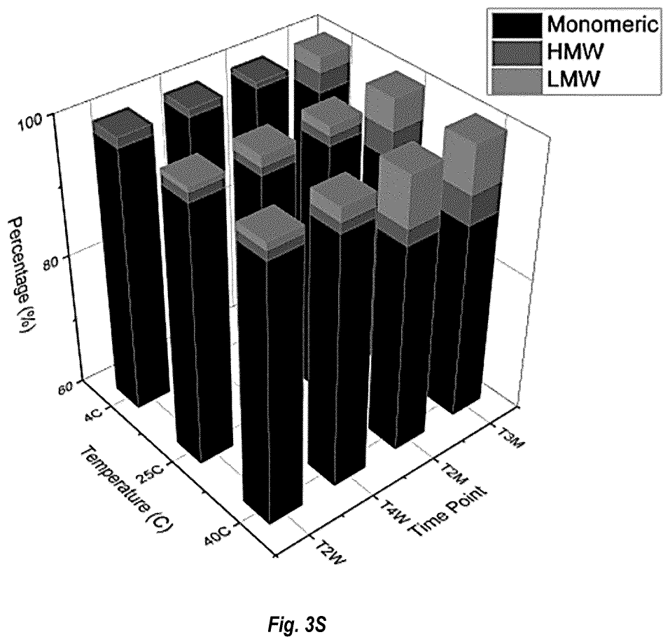

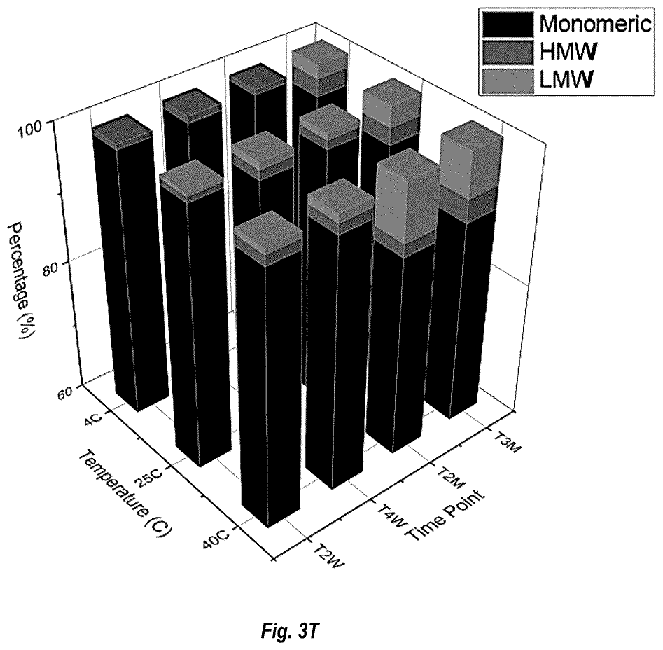

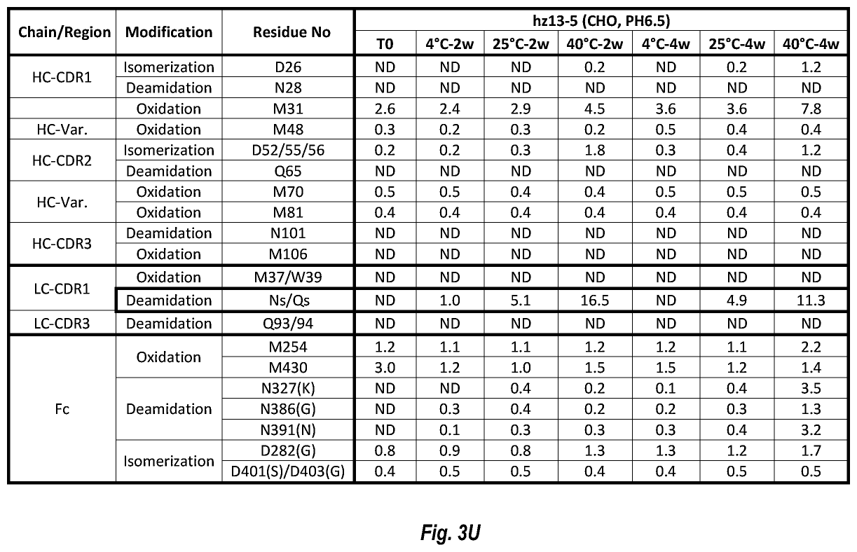

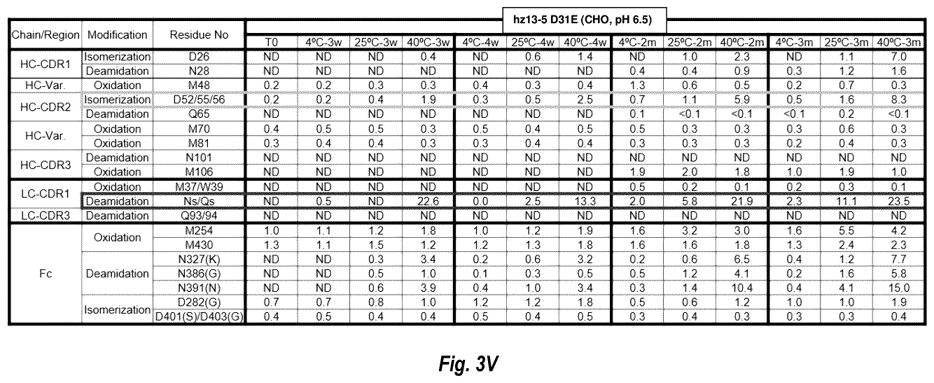

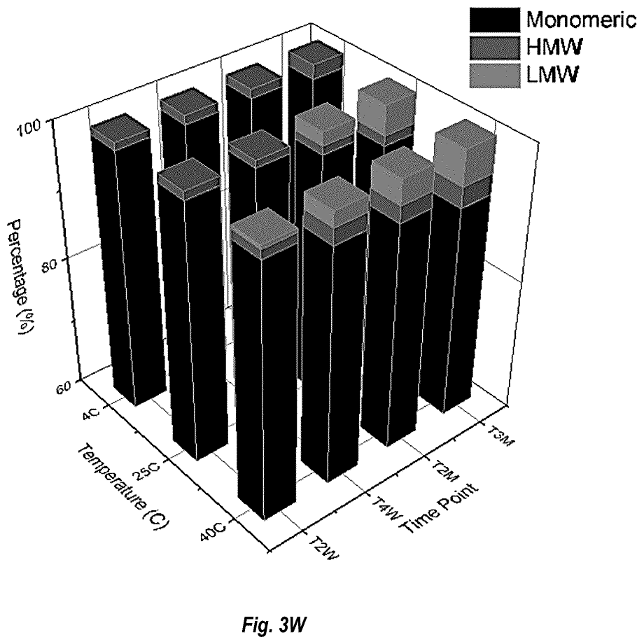

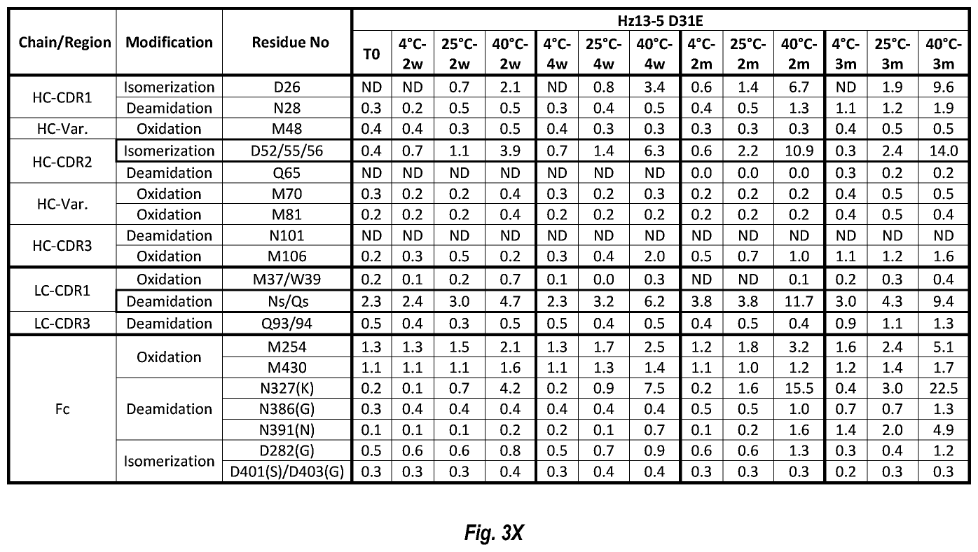

SEC. The SEC results are shown in FIG. 3A. The antibodies generally showed less than 5% drop in monomeric peak, at both 4° C. and 40° C. All of the antibodies showed very little, if any, change at 4° C. over the course of 4 weeks. At 40° C. they showed a significant increase in the low molecule weight (LMW) fragmentations and showed stable or slightly increased amounts of high molecular weight (HMW) aggregates compared to the starting time point.

Peptide Mapping. Four of the humanized clone 20 antibodies were examined by peptide mapping. The results indicated no substantial chemical modifications after storage for up to 4 weeks at 40° C. (FIG. 3B).

Four of the humanized clone 13 antibodies, hz13-5, hz13-10, hz13-11, and hz13-12, were examined by peptide mapping. The results showed that the humanized clone 13 antibodies had a propensity to isomerize, by as much as 74%, at D31 of heavy chain CDR1 (FIGS. 3C-3D). The isomerization observed at D31 was much less in the hz13-5 than in the other three clone 13 derived antibodies. Other locations did not show substantial chemical modifications (FIG. 3C). The D31 isomerization was notable but when the isomerized antibodies were examined by SPR, the isomerization did not affect the binding of the antibodies (data not shown).

### Example 8: Mitigating Isomerization at D31

Subsequent studies were conducted to evaluate approaches to mitigating the isomerization at position D31 of heavy chain CDR1 that was observed in the screening described above. The antibodies used in this Example had sequences as disclosed herein, including an IgG1.3 constant region, and were expressed and purified as described in Example 2.

A. pH Effect on Isomerization

1. Methods

A pH screen was performed on clone 13 derivatives hz13-5 and hz13-12. Since isomerization is thermal and pH dependent these two constructs were examined under controlled temperature conditions (4° C. or 40° C.) at pH 6, 6.5, 7.0, 7.5, and 8.0. The formulation buffer used was 20 mM histidine, 260 mM sucrose, 50 μM DTPA, 0.05% PS80 for pH 6.0 and pH 6.5. For pH 7.0, 7.5, and 8.0, the formulation buffer used was 20 mM sodium phosphate, 260 mM sucrose, 50 μM DTPA, 0.05% PS80. The samples were examined at a concentration of 1 mg/mL; 1 mL/vial of each antibody was added to a 2 mL screw cap clear vial (Agilent) and parafilm tape was secured around the top to insure a tight seal for the initial for the truncated stability study. The vials were stored in the appropriate incubator. 500 μL of the sample was removed from each vial at 2 and 4 weeks for analysis. Chemical liabilities were analyzed by size exclusion chromatography and peptide mapping. The SEC and peptide mapping were performed using methods as described in Example 7 above.

2. Results

The SEC results for hz13-12 and hz13-5 as a function of pH and time are shown in FIG. 3E and FIG. 3F respectively. With increasing pH, the amount of isomerization decreased as observed by peptide mapping. At pH 8.0 for both hz13-12 (FIGS. 3G-3J) and hz13-5 (FIGS. 3K-3N), the levels of isomerization were nondetectable. However, at pH levels of 6.5 or higher, deamidation was observed and the deamidation increased with increasing pH.

B. Effect of D31E Point Mutation

1. Methods

Another strategy that was investigated to reduce or eliminate the isomerization observed at position D31 of heavy chain CDR1 was to replace the aspartic acid at position 31 of heavy chain CDR1 with glutamic acid (D31E mutation). This results in a like-for-like charge substitution and because of the longer carbon chain on the R-group, the rate of isomerization may be reduced; this was confirmed experimentally.

Two such mutants were prepared: hz13-5 D31E and hz3-12 D31E. These mutants were produced in HEK cells, purified, and subjected to thermal stability testing over 2 months with testing performed at time points of TO (before storage) and at 2 weeks, 4 weeks, and 2 months following controlled temperature storage at 4° C., 25° C. and 40° C. in a Binder incubator. The samples were at a concentration of 50 mg/mL and in a formulation of 20 mM Histidine, 260 mM Sucrose, 50 μM DTPA, 0.05% PS80 for pH 6.0. 100 μL/vial of each antibody was added to a 2 mL screw cap clear vial (Agilent) with an insert and parafilm tape was secured around the top to insure a tight seal. The vials were stored in the appropriate incubator. 50 μL of the sample was removed from each vial at 2 weeks, 4 weeks, and 2 months for analysis by size exclusion chromatography and peptide mapping.

2. Results

The SEC results for hz13-12 D31E and hz13-5 D31E are shown in FIG. 3O and FIG. 3P respectively. The D31E mutant antibody samples incubated at 4° C. were stable with little to no change up to two months. The samples incubated at 40° C. show minor increases in LMW fragmentation, within acceptable limits. The peptide mapping results indicated that both hz13-12 D31E (FIG. 3Q) and hz13-5 D31E (FIG. 3R) showed acceptable chemical stability. At the elevated temperature of 40° C., there was a trend to increased isomerization at the D52/55/56 residues and deamidation at Ns/Qs residues. No substantial chemical modifications were observed at lower temperatures.

3. Conclusion

Increasing the pH to mitigate isomerization at position D31 tended to result in more deamidation in LC-CDR1, whereas the D31E point mutation did not result in this trade-off. The D31E mutant antibodies had acceptable chemical stability. Accordingly, the D31E mutation was identified as a favorable approach to mitigating isomerization at D31.

### Example 9: Stability of High Concentration Formulation

For subcutaneous administration, antibodies must be formulated at high concentrations. Two sets of high concentration screening experiments were performed.

High concentration thermal stability screenings of the hz13-5 parent and the hz13-5 D31E mutant were performed. The thermal stability study was over 3 months with time points of TO (time zero, before storage) and after controlled temperature storage at 2 weeks, 4 weeks, 2 months, and 3 months at 4° C., 25° C. and 40° C. in a Binder incubator. The antibodies were expressed in HEK or CHO cells, purified and formulated at a concentration of 150 mg/ml in 20 mM Histidine, 260 mM Sucrose, 50 μM DTPA, 0.05% PS80 at pH 6.5. 100 μL/vial of each antibody was added to a 2 mL screw cap clear vial (Agilent) with an insert and parafilm tape was secured around the top to insure a tight seal. The vials were stored in the appropriate incubator. 50 μL of the sample was removed from each vial at 2 weeks, 4 weeks, 2 months, and 3 months for analysis. Chemical liabilities were analyzed by SEC and peptide mapping. The SEC and peptide mapping were performed using methods as described in Example 7.

SEC was used to analyze the aggregation and fragmentation of the molecules over 3 months. Hz13-5 did not show changes in HMW or LMW until 3 months at 40° C., where a small increase was seen (FIG. 3S). At 25° C., a slight increase in LMW was seen at 2 weeks, 4 weeks, and 2 months. At 3 months both LMW and HMW increased but were within acceptable limits. At 40° C., a slight increase in LMW was seen at 2 weeks and 4 weeks, and at 2 months and 3 months, significant LMW occurred, as well as a slight increase in HMW. These were within acceptable limits. No significant changes were observed at 4° C. over at least 3 months. The same trends were seen for the hz13-5 D31E antibody (FIG. 3T).

Peptide mapping of hz13-5 parent was performed at 2 and 4 weeks at pH 6.5 (results shown in FIG. 3U). Under storage conditions (4° C.) the isomerization at position D31 was minimized. Peptide mapping of hz13-5 D31E was analyzed over three months (results shown in FIG. 3V). While there was an increase in chemical modification under stressed conditions, the antibody exhibited acceptable stability over 3 months at 4° C.

### Example 10: Stability of Purified Formulation of Hz13-5 D31E

Hz13-5 D31E was produced in CHO cells, purified, and formulated at a concentration of 54.6 mg/mL in 20 mM Histidine, 250 mM Sucrose, 50 μM DTPA, 0.05% PS80 at pH 6.0. 100 μL/vial of each antibody was added to a 2 mL screw cap clear vial (Agilent) with an insert and parafilm tape was secured around the top to insure a tight seal. The vials were stored in the appropriate incubator. The thermal stability study was over 3 months with time points of TO (time zero, before storage) and after controlled temperature storage at 2 weeks, 4 weeks, 2 months, and 3 months at 4° C., 25° C. and 40° C. in a Binder incubator. 50 μL of the sample was removed from each vial at 2 weeks, 4 weeks, 2 months, and 3 months for analysis. Chemical liabilities were analyzed by size exclusion chromatography (SEC) and peptide mapping. The SEC and peptide mapping were performed using methods as described in Example 7.

Hz13-5 D31E has an acceptable stability profile as the material exhibited minimal changes in SEC profile (FIG. 3W) and chemical stability at 4° C. (FIG. 3X).

### Example 11: Epitope Mapping of PAD4 for Humanized Clone 13 and Clone 20 Antibodies

The human PAD4 protein was analyzed for binding epitopes upon interaction with clone 13 and clone 20 antibodies. Methods used for this analysis were hydrogen/deuterium exchange mass spectrometry (HDX-MS), and orthogonal covalent labeling footprinting techniques, specifically fast photochemical oxidation of proteins (FPOP), glycine ethyl ester labeling (GEE), and diethylpyrocarbonate (DEPC).

A. Epitope Characterization Methods

Hydrogen/deuterium exchange mass spectrometry (HDX-MS) methods. First, non-deuterated experiments were carried out to generate a list of common peptides for recombinant human PAD4, and protein complexes of human PAD4 with the Fab of clone 13 (SEQ ID Nos: 226 and 228) or with the Fab of clone 20 (SEQ ID Nos: 229 and 231). The antibody concentrations were 15 μM and the protein to ligand molar ratio was a 1:1 molar ratio. In the HDX-MS experiment, 5 μL of each sample was diluted into 55 μL of D20 buffer (10 mM phosphate buffer, D₂O, pD 7.0) to start the labeling reactions. The reactions were carried out for different periods of time: 20 sec, 1 min, 10 min and 60 min. By the end of each labeling reaction period, the reaction was quenched by adding quenching buffer (100 mM phosphate buffer with 4 M GdnCl and 0.4 M TCEP, pH 2.5, 1:1, v/v). 50 μL of quenched sample was injected into Waters HDX-MS system for analysis. The deuterium uptake levels of common peptic peptides were monitored in the absence or presence of the antibodies.

Fast photochemical oxidation of proteins (FPOP) methods. Epitope mapping by FPOP was performed on recombinant human PAD4, protein complexes of human PAD4 with clone 13 Fab (SEQ ID Nos: 226 and 228), and protein complexes of human PAD4 with clone 20 Fab (SEQ ID Nos: 229 and 231) (10 μM, 1:1 molar ratio). A KrF excimer laser was used to generate hydroxyl radicals by the photolysis of H₂O₂. The excitation wavelength was set to be 248 nm to prevent laser-induced conformational changes in the protein. Immediately prior to labeling, 5 μL of histidine and 5 μL H₂O₂ were added to a sample of protein. The final volume of protein solution was 50 μL, the final concentration of histidine was 500 μM, and the final concentration of H₂O₂ was 15 mM. The sample was then injected into fused silica tubing with a UV transparent window. The laser was adjusted to 70 mJ/pulse at a frequency of 7.4 Hz. Both FPOP and no laser control experiments were performed in triplicate. Each replicate was collected in a microcentrifuge tube containing 11 μL of quenching solution (comprising 50 nM of catalase and 20 mM of methionine). The samples were denatured, reduced, alkylated, and digested with trypsin followed by LC/MS/MS analysis. The oxidation levels of peptides were monitored in the absence/presence of the antibodies. Only residues with statistically significant difference in % labeling between hPAD4 and hPAD4/Fab (based on student T-test p value <0.01) were considered protected residues.

GEE labeling methods. GEE labeling was initiated by mixing 10 μL of each 1 mg/mL sample (human PAD4, protein complexes of human PAD4 with clone 13 Fab, and protein complexes of human PAD4 with clone 20 Fab, with 1 μL of 2 M GEE and 1 μL of 50 mM 1-ethyl-3-(3-dimethylaminopropyl) carbodiimide (EDC) at room temperature for 10 min. The reaction was quenched by adding 10 μL of 1M ammonium acetate to sample. 17.5 μL of each GEE labeled sample was subjected to enzymatic digestion. The sample was denatured, reduced, alkylated, and digested with trypsin followed by LC/MS/MS analysis. Only residues with statistically significant difference in % labeling between hPAD4 and hPAD4/Fab (based on student T-test p value <0.01) were considered protected residues.

DEPC labeling methods. DEPC labeling was initiated by mixing 15 μL of each sample (human PAD4, protein complexes of human PAD4 with Fab of clone 13, and protein complexes of human PAD4 with Fab of clone 20 at 15 μM) with DEPC at a DEPC:protein molar ratio of 8:1. The labeling took place at 37° C. for 10 min followed by quenching with Imidazole at a ratio of 1:50. With respect to DEPC:imidazole molar ratio, the DEPC labeled samples were denatured, reduced, alkylated, and digested with trypsin or Glu-C followed by LC/MS/MS analysis. Only residues with statistically significant difference in % labeling between hPAD4 and hPAD4/Fab (based on student T-test p value <0.01) were considered protected residues.

B. Epitope Characterization Results

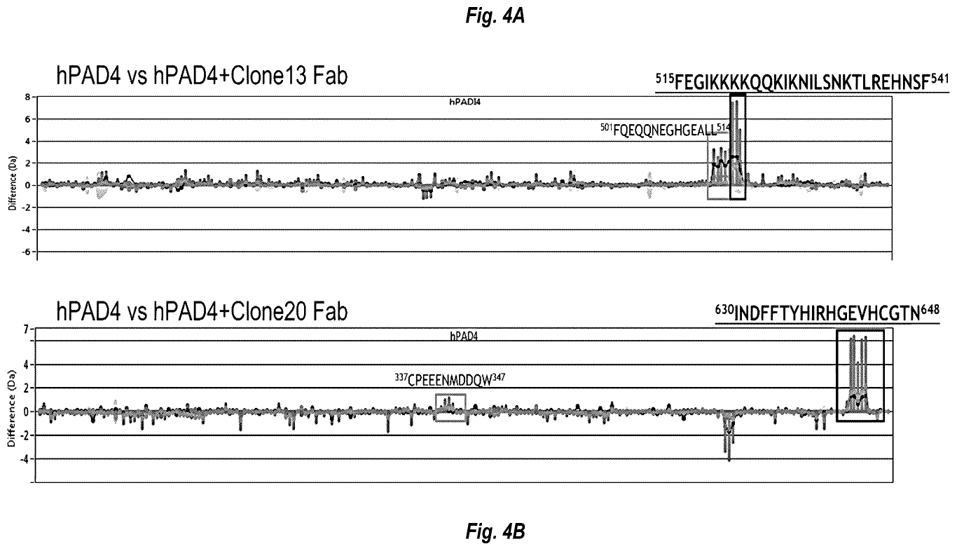

The epitopes of clone 13 and clone 20 were mapped using the methods described above. First, using HDX-MS, a sequence coverage of >98% was achieved for hPAD4. HDX-MS data analysis indicated that clone 13 and clone 20 have distinct epitopes in hPAD4 (FIG. 4A-FIG. 4B). Clone 13's primary HDX epitope is ⁵¹⁵FEGIKKKKQQKIKNILSNKTLREHNSF⁵⁴¹ (FIG. 4A; SEQ ID NO: 217). Clone 13's secondary HDX epitope: ⁵⁰¹FQEQQNEGHGEALL⁵¹⁴ (FIG. 4A; SEQ ID NO: 218). Clone 20's primary HDX epitope is ⁶³⁰INDFFTYHIRHGEVHCGTN⁶⁴⁸ (FIG. 4B; SEQ ID NO: 219). Clone 20's secondary HDX epitope is ³³⁷CPEEENMDDQW³⁴⁷ (FIG. 4B; SEQ ID NO: 220).

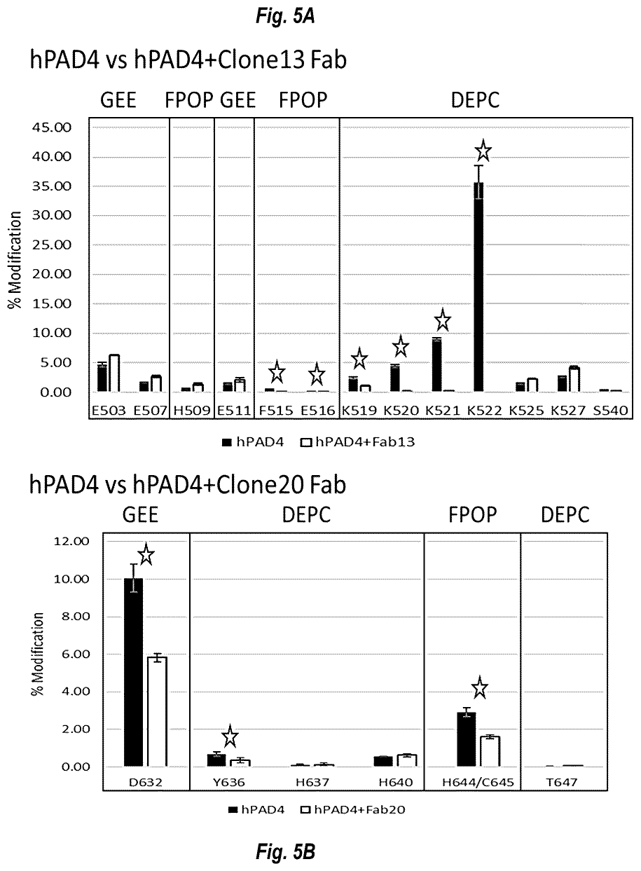

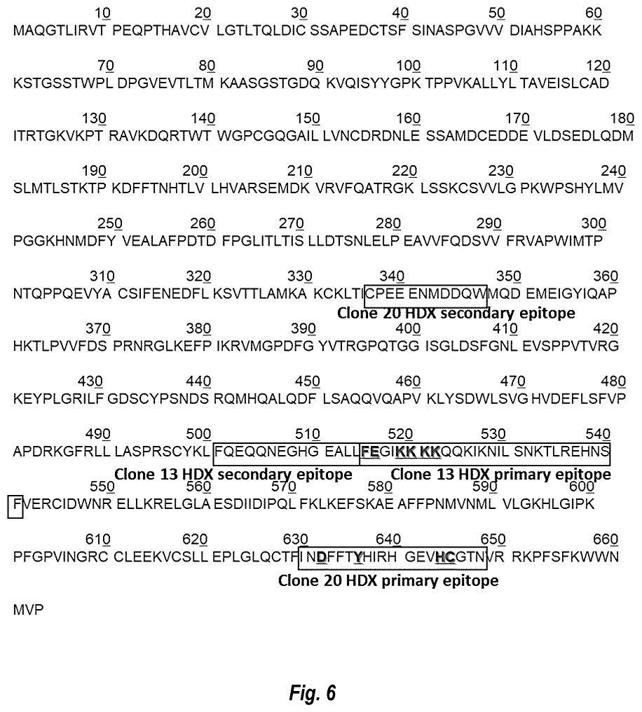

FPOP, GEE, and DEPC labeling methods demonstrated different residue-specific reactivities and provided complementary information across 100% sequence of hPAD4. Each of these labeling methods were performed separately and the % labeling from each method was examined across the entire sequence of hPAD4 with a focus on the epitope regions determined by HDX-MS as described above. The protected residues were determined based on the statistical significance of the difference in % labeling between hPAD4 and hPAD4/Fab (based on student T-test p value <0.01). FIG. 5A-FIG. 5B display the differential plots obtained from FPOP, GEE, and DEPC labeling experiments. The amino acid residues shown correspond to the labeled residues in the HDX epitope regions of clone 13 and clone 20. The protected residues with statistical significance were identified as follows: (1) protected residues by clone 13: F515, E516, K519, K520, K521, and K522; and (2) protected residues by clone 20: D632, Y636, and H644/C645. FIG. 6 shows the epitopes of clone 13 and clone 20 (as determined by HDX-MS, FPOP, GEE, and DEPC labeling) on corresponding protein sequences of hPAD4 (FIG. 6).

### Example 12: Identification of Paratopes on the Anti-PAD4 Clone 13 Antibody

This example describes the mapping of the paratope of anti-human PAD4 clone 13 parental antibody (mAb13) upon interaction with human PAD4.

A. HDX Methods for Paratope Mapping

Hydrogen/deuterium exchange mass spectrometry (HDX-MS) was utilized to probe the paratope, the binding site of mAb13, to human PAD4. Non-deuterated experiments were carried out to generate a list of common peptides for recombinant Fab of mAb13, and protein complex of human PAD4 with mAb13 (15 μM, 1:1 molar ratio). General HDX methods were as described in Example 11 above. The deuterium uptake levels of common peptic peptides were monitored in the absence or presence of human PAD4.

B. Paratope Mapping Results

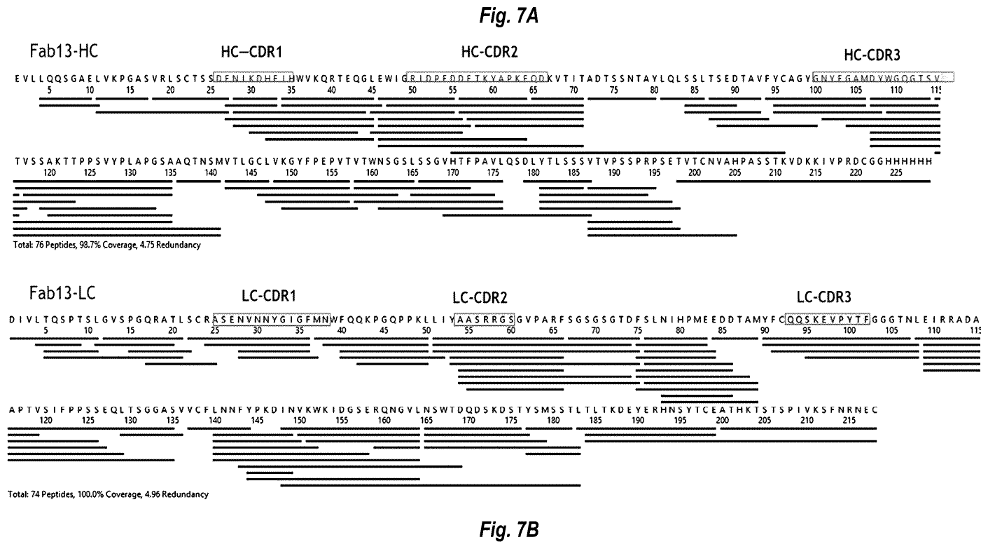

In mapping the paratope of mAb13, a sequence coverage >98% was achieved for both Heavy Chain (HC) and Light Chain (LC). Namely, a sequence coverage of 98.7%, with 4.75% redundancy was obtained for HC, and a sequence coverage of 100%, with 4.96% redundancy for LC (FIG. 7A-FIG. 7B). Paratope regions were determined based on the levels of HDX protection and are shown in FIG. 8A-FIG. 8B. For the HC, the first paratope region identified was ²⁷FNIKDHF³³ (SEQ ID NO: 221), which overlapped with the Heavy Chain Complementary Determining Region 1 (HC-CDR1) (FIG. 8A). The second paratope region identified for the HC was ⁹⁴FYCAGYGNYEGAMDY¹⁰⁸ (SEQ ID NO: 222, which overlapped with Heavy Chain Complementary Determining Region 3 (HC-CDR3) (FIG. 8A). For the LC, the first paratope region identified was ²⁶SENVNNYGIGFM³⁷ (SEQ ID NO: 223), which overlapped with Light Chain Complementary Determining Region 1 (LC-CDR1) (FIG. 8B). The other LC paratope region identified was ¹³⁷VCFLNNFY¹⁴⁴ (FIG. 8B; SEQ ID NO: 224) (which corresponds to the fragment VCLLNNFY (SEQ ID NO: 235) in an exemplary human LC constant region). The main paratope regions identified by HDX-MS were HC-CDR1, HC-CDR3 and LC-CDR1. Since all humanized clone 13 antibodies have the same CDRs as the parent clone 13, the paratope mapping data is applicable to all humanized variants of clone 13.

### Example 13: Cryo-Electron Microscopy (Cryo-EM) Structure of the PAD4.Clone 13 Fab Complex

Cryo-electron microscopy was used to determine the structure of a complex of PAD4 and the Clone 13 Fab.

A. Methods for Purification of PAD4 and Clone 13 Fab, and Methods for Complexation and Characterization of PAD4.Clone 13 Fab Complex

Human PAD4 (1-663) gene was cloned in pET28a vector (Novagen). PAD4 protein (N-His-TVMV-PAD4) (SEQ ID NO: 3) was expressed in Rosetta2 (DE3) pLysS E. coli cells. The cells were induced with 0.5 mM IPTG and grown overnight at 18° C. The protein was purified by Ni-NTA affinity chromatography followed by size-exclusion chromatography in the buffer 50 mM Tris, pH 8.5, 500 mM NaCl, 1 mM DTT, 1 mM EDTA.

Clone 13 Fab heavy chain (HC) and clone 13 Fab light chain (LC) were cloned in pTT5 vector, with a C-terminal His-tag in the HC (SEQ ID Nos: 226 and 228, respectively). Clone 13 Fab (comprising both HC and LC) was transiently expressed in Expi-293 cells. Clone 13 Fab was purified by Ni-NTA affinity chromatography followed by size exclusion chromatography in PBS.

For purification of the PAD4.clone 13 Fab complex, purified clone 13 Fab was mixed with PAD4 in three molar excess and incubated at 4° C. overnight. This was followed by size exclusion chromatography in a final buffer of 25 mM Tris pH 8.0, 250 mM NaCl, 1 mM DTT, and 1 mM EDTA.

B. Cryo-EM Methods

For grid preparation, 3 μL of 1 mg/mL PAD4.clone 13 Fab complex was applied to freshly glow discharged Quantifoil 0.6/1 300 mesh grid and blotted at 4° C. in 100% relative humidity for 4 seconds using a Vitrobot Mark IV (FEI) before plunge freezing in liquid ethane. Grids were then shipped to Beckman Center for Cryo-EM, Department of Biochemistry, University of Utah in a dry dewar, cooled to liquid nitrogen temperature, for data collection.

For data collection, 4999 high-magnification movie frames were recorded at University of Utah on a 300 kV Titan Krios microscope (Thermo Fisher Scientific), equipped with a GIF Quantum energy filter (Gatan) and a K3 Summit direct electron detector (Gatan). Movie frames were recorded using the EPU software with a calibrated pixel size 0.6723 Å and total dose ˜48e−/Å2 in the defocus range of −0.7 to −2.0 μm.

For data processing, the cryoSPARC package was used. Recorded movie frames were first dose-fractionated and corrected for beam induced motion using “Patch motion” from cryoSPARC. Contrast transfer functions (CTF) were calculated for all the motion corrected, aligned, and averaged movie frames using “Patch CTF” module of cryoSPARC. 4017 images were then selected based on CTF estimated resolution, ice thickness and total motion during motion correction. 564,384 particles were picked using a combination of blob picker and templated based picking as implemented in cryoSPARC. Picked particles were then subjected to 2D classification. 229,914 particles were selected after 2D classification. These selected particles were then classified in two pools using ab initio reconstruction program of cryoSPARC. The larger pool with 194,891 particles was then refined to ˜2.6 Å resolution. 194,891 particles were then further classified in two classes using another round of ab initio reconstruction. The larger pool of particles with 131,529 particles was then refined to −2.5 Å. All resolution estimates are based on the gold-standard Fourier-Shell Correlation threshold criterion of 0.143.

For model building and refinement, PAD4 and clone 13 Fab models were fitted into the cryoEM map using the UCSF Chimera software. The docked model was then subjected to iterative model building and real space refinement in Coot and Phenix respectively. Coot and Pymol were used for structural analyses.

C. CDRs at the PAD4.Clone 13 Fab Interface

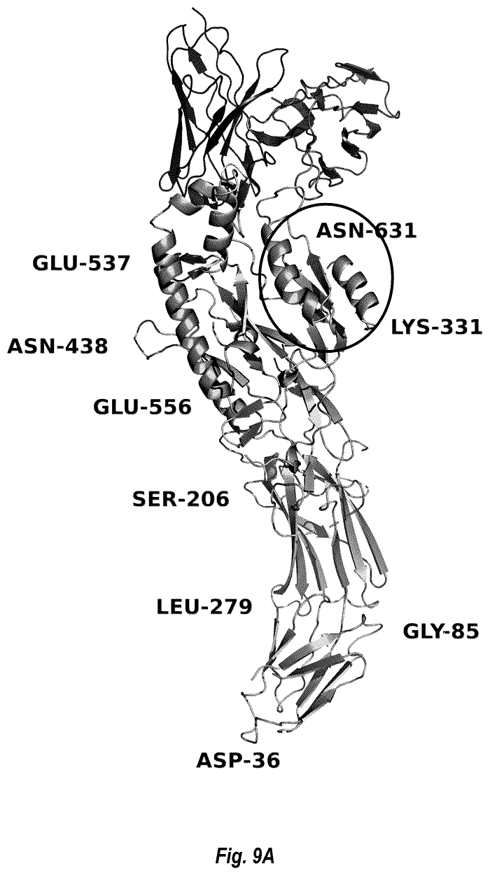

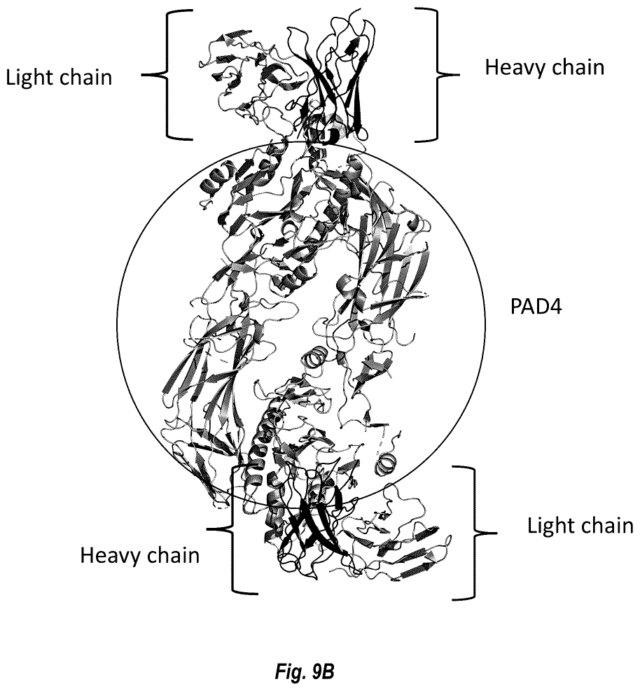

The PAD4.clone 13 Fab complex (FIG. 9A) was seen as a 2:2 (dimer) assembly (FIG. 9B). The heavy and light chain constant regions could not be resolved and cryoEM density for part of the VH and VL regions which lie away from the PAD4 interface was also not well resolved. However, the PAD4.clone 13 Fab interface was clearly defined.

The role and interactions of individual CDRs were identified. Each CDR contributed to the surface area of clone 13 Fab that was buried upon clone 13 Fab complexation with PAD4. All three of the CDRs from the heavy chain (CDR1, CDR2, and CDR3 in the HC) were observed to interact with PAD4. In contrast, in the LC, only CDR1 made significant interactions with PAD4. The contribution of individual CDRs to the interaction with PAD4, in terms of buried area on complexation, is given in Table 8 below. LC CDR1 made primarily hydrophobic/van der Waal interactions with PAD4 with Y28-hydroxyl of LC CDR1 forming a hydrogen bond with Gly603 of PAD4.

TABLE 8
CDRs of Clone 13 Fab.

| CDRs | % Buried area | Area (Å²) |
|---|---|---|
| LCDR1 | 34 | 225 |
| HCDR3 | 23 | 153 |
| HCDR2 | 15 | 99 |
| HCDR1 | 11 | 71 |
| LCDR2 | 8 | 54 |
| LCDR3 | 4 | 29 |

D. Clone 13 Fab's Mechanism of Action in Inhibiting PAD4

Analysis of the cryo-EM structures described in this Example provide a plausible inhibitory mechanism of action. In the substrate peptide bound form, the lysine loop of PAD4 (amino acids 510-526 of PAD4, which contains the tetra lysine stretch at amino acids 519-522 of PAD4), acted as a lid to the coil formed by amino acids 630-638. The coil at amino acids 630-638, which may be important for proper positioning of the substrate in the active site, is thus stabilized by the lysine loop. (FIG. 11; adapted from Protein Databank ID 2DEW (a submitted crystal structure of human peptidylarginine deiminase 4 in complex with histone H3 N-terminal tail including Arg8; hereinafter (“2DEW”) as provided at rcsb.org/structure/2DEW; files.rcsb.org/pub/pdb/validation reports/de/2dew/2dew_full_validation.pdf; and PNAS (2006) 103, 5291-5296).

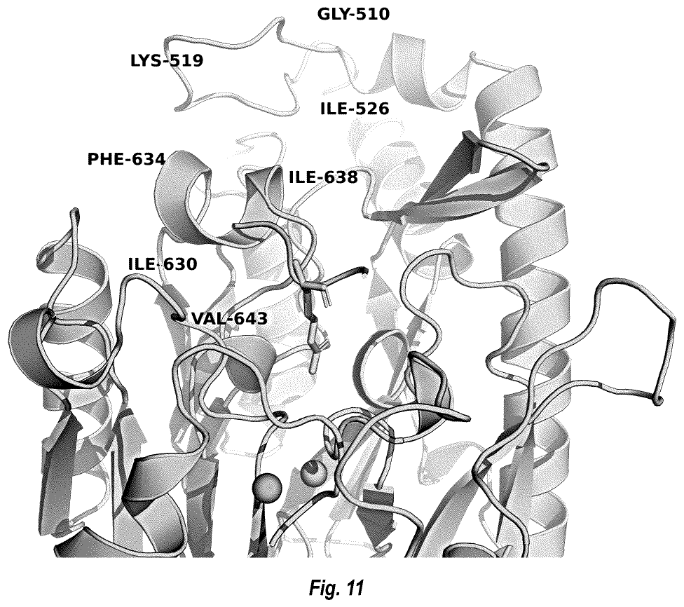

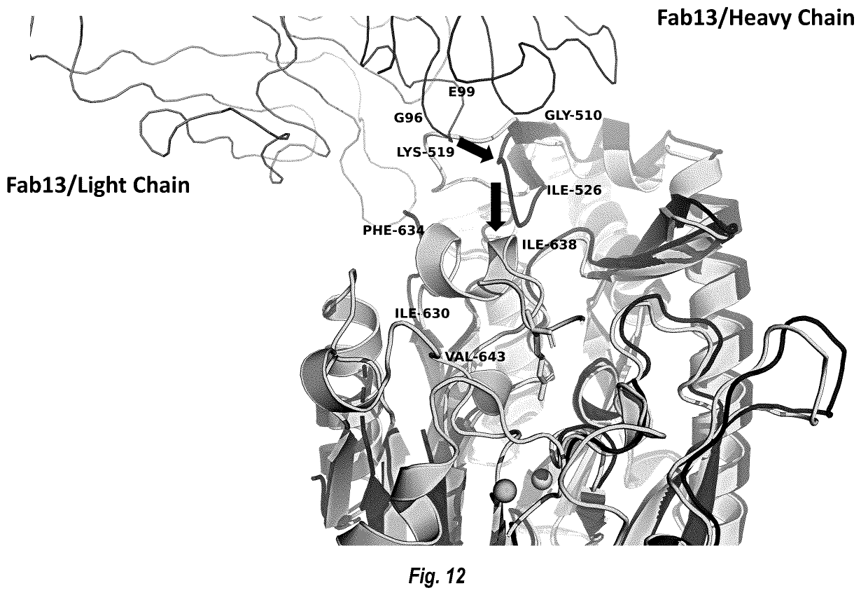

Further, in the cryoEM structure of the PAD4.clone 13 Fab complex (FIG. 12), the antibody induced reconfiguration of the PAD4 lysine loop. In particular, HC CDR3 dislodged the PAD4 lysine loop and generated a new epitope surface for clone 13 Fab binding. The dislodged lysine loop, now involved in direct interactions with the antibody, bumps into the 630-638 catalytic coil and causes disorder in the coil. This disorder is expected to destabilize the substrate binding site and inhibit PAD4 enzymatic activity. An ordered mobile loop 630-643, as in 2DEW, is not sterically compatible with clone 13 Fab binding.

These cryoEM structure results are consistent with the HDX paratope mapping results discussed in Example 12 above, which indicate that HC-CDR1, HC-CDR3 and LC-CDR1 are the primary paratopes.

E. pH-Dependent Anti-PAD4 Antibody

The cryo-EM structures described in this Example were also analyzed to understand the structural basis for pH-dependence of an anti-PAD4 antibody (see Example 14 below).

### Example 14: Design and Characterization of pH Dependent Anti-PAD4 Antibodies Based on Parental Antibody hz13-5

Antibody mutants were prepared to obtain a pH dependent anti-PAD4 antibody that retains binding to PAD4 at neutral or physiological pH, such as pH 7.4, but that has weaker binding to PAD4 at acidic pH, such as pH 6.0. Such an antibody is expected to retain binding and complexation with PAD4 in environments such as synovial fluid, but once internalized into cells, it is expected to dissociate from PAD4 in the acidic compartment of the endosome, causing PAD4 to be shut to the lysosome for degradation and the antibody to be recycled back to the surface via binding to FcRn; accordingly, pH dependence allows reduced degradation of the antibody.

A. Mutational Scan Library Methods

A mutational scan library was designed such that each member of the library contained only a single mutation within the complementarity-determining region (CDR) of the antibody. Each position within the CDR was mutated to any of the 20 possible amino acid residues, and the library was designed to scan all 6 CDRs of the antibody in this manner. Accordingly, a total of 1360 single mutation antibodies were generated a clone 13-based parental antibody hz13-5. The mutational scan library was constructed as scFvs using overlapping PCR (Xu L et al. (2002) Chemistry & Biology 9: 933; Roberts R W and J W Szostak (1997) Proc. Natl. Acad. Sci. USA 94:12297; Kurz et al. (2000) Nucleic Acids Res. 28(18):E83). To ensure only one mutation per library member, two mutational scan libraries were generated: one for HC and another for LC. For example, if the mutational scan was in the HC CDRs, then the LC sequence was held constant.

In vitro selection of the mutational scan libraries was carried out using mRNA display, with the individual member made into mRNA-protein fusion molecules using methods previously described (Xu L et al. (2002) Chemistry & Biology 9: 933; Roberts R W and J W Szostak (1997) Proc. Natl. Acad. Sci. USA 94:12297; Kurz et al. (2000) Nucleic Acids Res. 28(18):E83). Prior to the selection, an aliquot of the fusion molecules for each library was set aside for downstream analysis (“input”). The libraries were then selected for binding against 1 nM biotinylated human PAD4 at pH 7.4 and at pH 6, and binding members were eluted from the streptavidin beads using KOH. Lastly, the cDNA portion of the molecule of the eluted fraction, as well as the input fraction, were amplified for analysis by next generation sequencing (NGS).

Pair-end 2×300 runs were utilized for sequencing either the heavy chain or the light chain libraries. Forward and reverse reads were paired, trimmed for quality score of Q>30, binned by barcode for individual sample and selection conditions, filtered for full-length HC or LC sequence, and filtered for either wildtype or variants containing only a single mutational substitution within the CDR of either the HC or the LC. Sequences that did not fit the above criteria were removed from further analysis. The frequency of each library member in the eluted fraction was divided by the frequency of each library member in the input fraction to derive an enrichment ratio (ER). This value was further normalized against that of the wildtype sequence to allow for comparison of each variant against the parental sequence to assess for tolerance of a particular substitution at that position. Variants with an ER value of close to or equal to 1 (i.e., 0.5<ER<1.5) have a neutral mutation, in which the amino acid substitution is tolerated for binding, whereas those with an ER value of less than 0.5 have a mutation that negatively impacts binding. The calculated ER ratios for individual members of the library were represented as heat maps to readily identify variants that have comparable binding to the wildtype sequence at pH 7.4, and, in contrast, reduced binding compared to the wildtype sequence at pH 6.

B. Results

Based on the heatmap analysis (FIGS. 13A-13L), a set of variants was selected for further characterization as full IgGs. These variants contained either a single mutation in which the ER indicated weaker binding at pH 6, or combination of such mutations that was selected for the purpose of assessing whether the combination would yield a greater loss of binding at pH 6. The selected mutations were put into the clone 13-based antibody hz13-5. This resulted in the 31 variants listed in Table 9 below, which were tested for pH-dependent binding to human PAD4. The mutations listed are relative to the clone 13-based antibody hz13-5. The full variable region sequences of these 31 variants are provided in the Sequence Table below.

TABLE 9
Variants of the hz13-5 antibody.

| Antibody | VH mutation | Vk mutation |
|---|---|---|
| hz13-5 VH P52aK | P52aK | None |
| hz13-5 VH I34H | I34H P52aK | None |
| P52aK | | |
| hz13-5 VH I34H | I34H H35K P52aK | None |
| H35K P52aK | | |
| hz13-5 VH I34H | I34H H35K | None |
| H35K | | |
| hz13-5 VH I34H | I34H | None |
| hz13-5 VH H35K | H35K P52aK | None |
| P52aK | | |
| hz13-5 VH H35K | H35K | None |
| hz13-5 VH H32W | H32W | None |
| hz13-5 VH D31H | D31H P52aK | None |
| P52aK | | |
| hz13-5 VH D31H | D31H I34H P52aK | None |
| I34H P52aK | | |
| hz13-5 VH D31H | D31H I34H H35K | None |
| I34H H35K P52aK | P52aK | |
| hz13-5 VH D31H | D31H I34H H35K | None |
| I34H H35K | | |
| hz13-5 VH D31H | D31H I34H | None |
| I34H | | |
| hz13-5 VH D31H | D31H H35K P52aK | None |
| H35K P52aK | | |
| hz13-5 VH D31H | D31H H35K | None |
| H35K | | |
| hz13-5 VH D31H | D31H | None |
| hz13-5 VH | P52aK | I30H |
| P52aK::Vk_I30H | | |
| hz13-5 VH_134H | I34H P52aK | I30H |
| P52aK::Vk_I30H | | |
| hz13-5 VH_I34H | I34H H35K P52aK | I30H |
| H35K | | |
| P52aK::Vk_I30H | | |
| hz13-5 VH_I34H | I34H H35K | I30H |
| H35K::VK_I30H | | |
| hz13-5 | I34H | I30H |
| VH_I34H::Vk_I30H | | |
| hz13-5 VH_H35K | H35K P52aK | I30H |
| P52aK::Vk_I30H | | |
| hz13-5 | H35K | I30H |
| VH_H35K::Vk_I30H | | |
| hz13-5 VH_D31H | D31H P52aK | I30H |
| P52aK::Vk_I30H | | |
| hz13-5 VH_D31H | D31H I34H P52aK | I30H |
| I34H | | |
| P52aK::Vk_I30H | | |
| hz13-5 VH_D31H | D31H I34H H35K | I30H |
| I34H H35K | P52aK | |
| P52aK::Vk_I30H | | |
| hz13-5 VH_D31H | D31H I34H H35K | I30H |
| 134H | | |
| H35K::Vk_I30H | | |
| hz13-5 VH_D31H | D31H I34H | I30H |
| 134H::Vk_I30H | | |
| hz13-5 VH_D31H | D31H H35K P52aK | I30H |
| H35K | | |
| P52aK::Vk_I30H | | |
| hz13-5 VH_D31H | D31H H35K | I30H |
| H35K::Vk_I30H | | |
| hz13-5 | D31H | I30H |
| VH_D31H::Vk_I30H | | |

Of the above variants tested, only one variant, hz13-5 VH_D31H::Vk_I30H, demonstrated pH dependent binding to human PAD4. The binding activity of this mutant at pH 7.6 was comparable to (unmutated) hz13-5 binding to human PAD4 (see FIG. 13M). Whereas the parent hz13-5 retained binding at pH 6.0, the mutant hz13-5 VH_D31H::Vk_I30H demonstrated a loss of binding to human PAD4 at pH 6.0. See FIG. 13N.

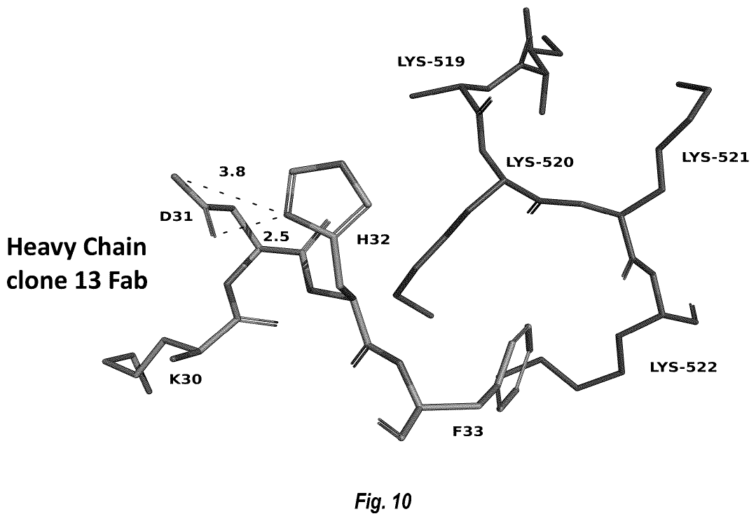

The cryoEM structure of the clone 13 Fab was analyzed to understand the structural basis for pH dependent binding of hz13-5 VH_D31H::Vk_I30H. For example, the sidechain of D31 was observed to interact with the sidechain of adjacent H32 (see FIG. 10). D31 is also adjacent to K30. This K30-D31-H32 region in the HC is proximal to tetra lysine stretch (amino acids 519-522 of PAD4) of the lysine loop (amino acids 510-526 of PAD4). In acidic conditions, an exemplary anti-PAD4 that comprised a D31H mutation would be positively charged and K30-D31H-H32 residues would repel each other. The repulsion would destabilize the PAD4-antibody interactions. In the LC of clone 13 Fab, 130 resides in a hydrophobic environment. An I30H mutation would introduce a polar residue in that hydrophobic environment, which could also destabilize the PAD4-antibody interface.

### Example 15: Anti-PAD4 Clone 13 and Clone 20 Antibodies Demonstrated Low Immunogenicity

Because therapeutic proteins administered to a patient may produce peptide antigens recognized by the patient's immune system as foreign antigens, they can elicit an undesired immune response, usually manifested by generation of anti-drug antibodies (ADA). The first step in this process is when an HLA class II molecule on an antigen-presenting cell (APC), such as a dendritic cell (DC), binds a peptide antigen. This peptide-major histocompatibility complex (MHC) complex can be recognized by CD4 T cells which will ultimately induce differentiation of B cells into plasma cells and result in the production of ADA.

Methods below were used to characterize the immunogenicity of the clone 13-based hz13-12 antibody and the clone 20-based hz20-7 antibody.

A. Methods for In Silico HLA Binding

In silico human leukocyte antigen (HLA) binding tools were used to model and predict antigen binding to MEW class II by sequentially ranking binding of overlapping 15meres spanning the antibody across multiple HLA alleles covering the genetic diversity within the human population (Wang et al., PLoS Comput Biol, 2008, 4(4): p. e1000048.).

A commercial in silico immunogenicity risk assessment algorithm (Epivax) was used to rank peptide MEW class II binding across 8 human HLA DRB1 allele super types to cover >90% of the variability present in the human population (De Groot and Martin, Clin Immunol, 2009, 131(2): p. 189-201.).

B. Methods for In Vitro DC-T Cell Assay

After an HLA class II molecule binds a peptide antigen, the next critical step in developing an immune response to a therapeutic antibody is the activation of CD4+ T cells. This T-cell activation occurs as a result of the recognition of a cognate peptide-MHC complex (HLA) on an antigen presenting cell (APC). In vitro peripheral blood mononuclear cell (PBMC) assays, using diverse donor sets, were used to determine whether a molecule contains functional T cell epitopes based on its ability to stimulate antigen-specific CD4+ T cells in vitro (Joubert et al., 2016, 11(8): p. e0159328).

An in vitro dendritic cell/T-cell (DC:T-cell) proliferation assay was conducted with hz20-7, hz13-12, hz13-5, and hz13-5 D31E for T cell epitopes capable of activating naïve T cells. Briefly, PBMCs from healthy volunteers were isolated by Ficoll (GE Healthcare, Chicago, IL) gradient centrifugation and HLA typed using polymerase chain reaction (PCR) amplification and hybridization with oligonucleotide probes (ProImmune, Sarasota, FL). A panel of 40 PBMC donors composed of HLA-DR class II alleles closely matching the world population frequencies was used for an assay run.

Monocytes were isolated from PBMC using a negative selection bead-based method (Miltenyi Biotec Inc, Bergisch Gladbach, Germany) and cultured for 3 days in DC media (Lonza, Basel, Switzerland) containing Interleukin 4 (IL-4) and granulocyte-macrophage colony stimulating factor (GM-CSF) to generate immature DCs. These cells were pulsed with (1) the clone 13-based hz13-12 antibody, (2) the clone 20-based hz20-7 antibody, (3) Avastin (anti-VEGF monoclonal antibody bevacizumab) as a control antibody that demonstrates low immunogenicity (Hua et al., J Clin Pharmacol, 2014, 54(1): p. 14-22), or (4) IL-21R mAb (AT-107, a fully human anti IL-21R monoclonal antibody) as a control antibody that demonstrates high immunogenicity. After an overnight incubation, the cells were thorough washed, and the cells were incubated overnight in media containing TNF-α, IL-1β, IL-6, and PGE2.

2,000 pulsed mature DCs were added to 200,000 autologous PBMCs-labeled with carboxyfluorescein succinimidyl ester (CFSE) (Invitrogen, Carlsbad, CA) to monitor proliferation and plated in 96-well plates using six replicates and DC media containing pen-strep (Gibco, Waltham, MA). After seven days, media was washed away and cells were labeled with an anti-human CD4+ APC (BD Biosciences, San Jose, CA) monoclonal antibody. Unbound anti-CD4 monoclonal antibody was removed with a wash step. Cells were fixed with 3.7% formalin (Sigma, St. Louis, MO) in phosphate-buffered saline (PBS) and analyzed by flow cytometry to determine the percentage of proliferating antigen-specific CD4+ T cells.

C. Immunogenicity Results

The in silico immunogenicity results are shown in Table 10. These results indicate that the humanized anti-PAD4 antibodies have low immunogenicity risk. The anti-PAD4 antibodies were found to be less immunogenic than various commercial antibodies (see comparator antibodies at bottom of table).

TABLE 10
In Silico Immunogenicity Results.

| | | | Epivax Ab |
|---|---|---|---|
| | | | Immunogenicity |
| Antibody | Epivax HC | Epivax LC | Scale |
| hz20-1 | −39.86 | −21.44 | 0.72% |
| hz20-2 | −40.29 | −34.61 | 0.19% |
| hz20-3 | −35.73 | −34.61 | 0.30% |
| hz20-4 | −41.95 | −21.44 | 0.58% |
| hz20-5 | −29.51 | −21.44 | 1.72% |
| hz20-6 | −17.96 | −34.61 | 1.67% |
| hz20-7 | −39.72 | −22.61 | 0.70% |
| hz20-8 | −39.72 | −17.97 | 1.49% |
| hz20-9 | −39.24 | −22.61 | 0.84% |
| hz20-10 | −39.24 | −17.97 | 1.71% |
| hz20-11 | −36.86 | −22.61 | 1.79% |
| hz20-12 | −36.86 | −17.97 | 3.04% |
| hz20-13 | −36.85 | −22.61 | 1.80% |
| hz20-14 | −36.85 | −17.97 | 3.05% |
| hz13-1 | −69 | −47.81 | 0% |
| hz13-2 | −69 | −35.19 | 0% |
| hz13-3 | −69 | 2.71 | 0.35% |
| hz13-4 | −54.33 | −47.81 | 0% |
| hz13-5 | −54.33 | −35.19 | 0% |
| hz13-6 | −54.33 | 2.71 | 1.45% |
| hz13-7 | −46.85 | −47.81 | 0% |
| hz13-8 | −46.85 | −35.19 | 0% |
| hz13-9 | −46.85 | 2.71 | 2.40% |
| hz13-10 | −41.88 | −11.22 | 1.49% |
| hz13-11 | −42.12 | −9.85 | 1.51% |
| hz13-12 | −42.12 | −11.22 | 1.38% |
| hz13-5 D31E | −55.30 | −37.60 | 0% |
| Campath | | | 45% |
| Rituxan | | | 27% |
| Zenapax | | | 14% |
| Humicade | | | 7% |
| Mylotarg | | | 2.90% |
| Avastin | | | 0% |

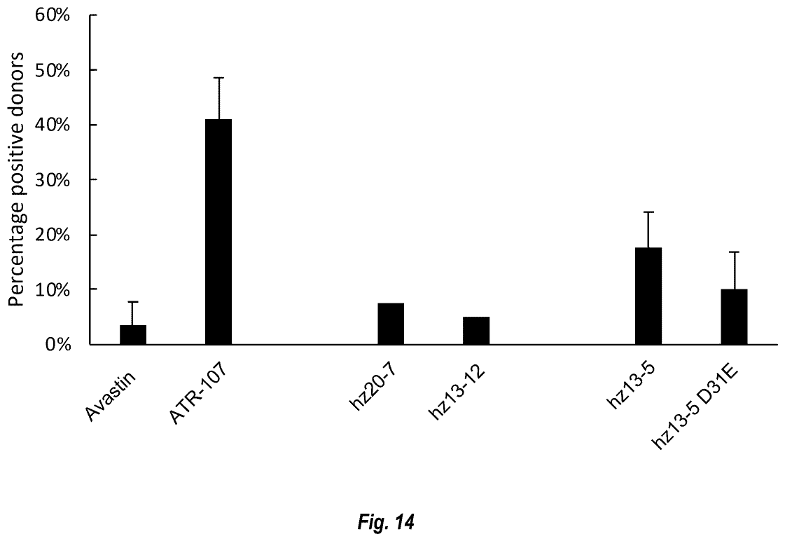

The in vitro assay results are shown in FIG. 14. These results similarly indicate that hz20-7 and hz13-12 demonstrated low immunogenicity.

In an in vitro DC-T cell assay utilizing a diverse set of 40 healthy PBMC donors the hz20-7, hz13-12, hz13-5 and hz13-5 D31E antibodies showed CD4+ T cell proliferative responses in 7.5%, 5%, 18% and 10% of the donors respectively. See FIG. 14. The results for hz20-7, hz13-12, and hz13-5 D31E were comparable to the 3.5% for the low control monoclonal antibody (Avastin, bevacizumab), which has been demonstrated to have low immunogenicity in the clinic. The 18% response rate for hz13-5, although higher than the historical range for Avastin (5-12.5%), falls within the range of low immunogenicity risk (<20%). (Hua, F., Comer, G. M., Stockert, L., et al. Anti-IL21 receptor monoclonal antibody (ATR-107): Safety, pharmacokinetics, and pharmacodynamic evaluation in healthy volunteers: A phase I, first-in-human study. The Journal of Clinical Pharmacology, 2014, 54: 14-22. doi:10.1002/jcph.158.)

The high control monoclonal antibody (ATR-107) showed CD4+ proliferation in 41% of donors, and has been shown to have a high ADA rate of 76% in clinical studies. (Hua, F, Comer, G M, Stockert, L, et al. Anti-IL21 receptor monoclonal antibody (ATR-107): Safety, pharmacokinetics, and pharmacodynamic evaluation in healthy volunteers: A phase I, first-in-human study. The Journal of Clinical Pharmacology. 2014, 54:14-22.)

The risk of hz20-7, hz13-12, hz13-5 and hz13-5 D31E antibodies to elicit an undesirable immune response in humans against the antibodies was determined to be low based on in silico sequence analysis and in vitro DC:T cell proliferation assays.

### Example 16: Anti-PAD4 Antibodies Hz13-5 and Hz13-5 D31E Inhibit PAD4 Activity In Vitro

Antibodies were next tested for their ability to inhibit the activity of PAD4 on a substrate in vitro.

A. Methods for ELISA

Microtiter plates (96 well Nunc MaxiSorp™ ELISA plates; Thermo Fisher Scientific Cat #44-2404-21) were coated with 1 μg/ml arginine-containing linear peptide synthesized by GenScript® (SHQESTRGKSKGKAAAAA; SEQ ID NO: 232) in PBS and incubated overnight at 4° C. Plates were washed three times with ELSA washing buffer (Cayman Cat #400062, Cat #400035). In a buffer containing 50 mM NaCl, 2 mM CaCl₂) 1 mM DTT (Invitrogen, Cat #P/N 46-2250) and 25 mM HEPES (Gibco Cat #15630-080), recombinant human PAD4 (rhPAD4) (Cayman Chemical #10500, Ann Arbor, MI) was preincubated with at concentrations of 13.5 nM (1 μg/mL), 27 nM, 54 nM, and 108 nM (8 μg/mL). Each preincubated solution was then mixed with an equal volume of anti-PAD4 antibody hz13-5 D31E or isotype control antibody hIgG1.3f, which was serial-diluted in the assay buffer to achieve antibody concentrations ranging from 0.13 nM to 66.7 nM. The recombinant PAD4 and antibody mixtures were incubated for 60 min at 4° C. 100 μl of each reaction was added into wells of peptide-coated microtiter plates. Plates were incubated at 37° C. overnight, and then washed three times with a washing buffer, and blocked for 1 hour with blocking buffer (Invitrogen Cat #DS98200). Wells were then further washed with washing buffer and incubated with 100 μl of horse radish peroxidase (HRP) conjugated anti-citrulline monoclonal antibody Clone 1D9 (Cayman chemicals, Cat #30773, 1:2000 in PBS-0.05% Tween®-20) for 1.5 hrs at room temperature. Subsequently, The plates were washed three times in washing buffer and incubated with a peroxidase substrate (TMB). After 30 minutes, the color reaction was stopped by adding 2N sulfuric acid (VWR Cat #VW3500-1). Optical density (OD) was measured at 450 nm using the SpectraMax™ 190. Data were acquired using Soft Max Pro™ 7.1.

The HRP conjugated anti-citrulline monoclonal antibody, clone1D9, was made using HRP conjugation kit (Abcam Cat #ab102890) by following manufacture's protocol. 10 ul of Modifier reagent was added into 90 ul of anti-citrulline antibody. The mixture was added directly onto the lyophilized HRP mix. Vials were left standing for 3 hrs in the dark at room temperature. After incubation, 10 ul of Quencher reagent was added and the solution mixed gently. The conjugates were used after 30 minutes without further purification.

All assays were performed in triplicate. All data are shown as means and ranges of triplicate measurements. Percentage of inhibition was calculated by percentage reduction of OD values from indicated concentration of PAD4 alone, after subtracting the background OD. The results are presented as IC50 values, which were calculated using GraphPad® Prism 9.4.0 (GraphPad Software, San Diego, CA). IC50s were determined by nonlinear regression curve fit with One site—Fit log IC50.

B. hz13-5 D31E Inhibited PAD4 In Vitro

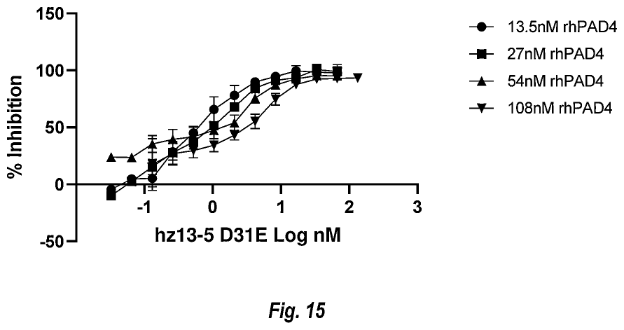

Antibody hz13-5 D31E inhibited PAD4 activity in a dose-dependent manner, as determined by citrulline ELISA. The IC50s proportionally increased with increased concentration of rhPAD4. Representative curves with 13.5 nM (1 μg/ml) and 108 nM (8 μg/ml) of rhPAD4 are shown in FIG. 15. The isotype control antibody showed no effect against rhPAD4 up to 66.7 nM for concentrations of rhPAD4 at 13.5 nM, 27 nM and 54 nM; and up to 133 nM for concentration of rhPAD4 at 108 nM. The experiment was repeated three times. Table 11 shows the IC50 for each run of the experiment, followed by the average and standard deviation for the three runs, showing that hz13-5 D31E inhibits citrullination of arginine in the tested peptide with IC50 values from about 0.5 to 5.0 nM, depending on the concentration of rhPAD4.

Similar experiments were also performed with the hz13-5 antibody (IC50 of 0.70+/−0.34 at 13.5 nM rhPAD4 to 5.02+/−0.88 at 108 nM rhPAD4) and with an anti-murine PAD4 antibody described below in Example 19.

TABLE 11
IC50 of Anti-PAD4 Antibody hz13-5 D31E.

| rhPAD4 | 13.5 nM | 27 nM | 54 nM | 108 nM |
|---|---|---|---|---|
| IC50 (nM) run 1 | 0.34 | 1.77 | 3.13 | 6.03 |
| IC50 (nM) run 2 | 0.73 | 1.26 | 3.14 | 4.29 |
| IC50 (nM) run 3 | 0.37 | 0.89 | 2.68 | 4.65 |
| Average +/− SD | 0.48 +/− | 1.31 +/− | 2.98 +/− | 4.99 +/− |
| | 0.22 | 0.44 | 0.26 | 0.92 |

### Example 17: Clone 13-Based Anti-PAD4 Antibodies Reduce Citrullinated Histones and Reduce Cytokine Expression and Secretion

In this Example, anti-PAD4 antibodies were analyzed for ability to reduce extracellular citrullinated H3 and secretion of cytokines in human blood monocytes stimulated by lipopolysaccharide (LPS).

A. Methods for the LPS-Stimulated Monocyte Assay

Several clone 13-based antibodies were tested. As in other Examples (unless otherwise specifically described), the antibodies were formatted with an IgG1.3f constant region (SEQ ID NO: 178). The antibodies were as follows: (1) hz13-5, (2) hz13-5 D31E, (3) hz13-3, (4) hz13-12, (5) hz20-2, (6) hz20-7, (7) Isotype control hIgG1.3f.

Human monocytes (CD14+CD16−) were isolated from fresh human PBMCs by immunomagnetic negative selection using EasySep™ Human Monocyte Isolation Kit (StemCell, Cat. #19359). The isolated human CD14+ monocytes were washed and cultured in Assay medium IMDM (Gibco, Cat. #31980-030), and 10% fetal bovine serum (FBS).

7×10⁴ of human CD14+ monocytes added to each well of a 96-well u-bottom polystyrene plate. The monocytes were incubated with different concentrations of clone 13-based IgG1.3f antibodies or the isotype control antibody, and 10 μg/mL LPS. The incubation volume in each well was 200 μL. The plate was incubated at 37° C., 5% CO₂ for 24 hrs.

The supernatant was collected for detection of extracellular citrullinated histone H3 (Cit-H3) by ELISA kit (Cayman Chemical, Cat. #501620), and for detection of secreted GM-CSF and other cytokines by Alphalisa (Perkin Elmer, Cat. #AL216). The cell lysates were used to isolate mRNA for gene expression by qRT-PCR. Data was analyzed using Excel and GraphPad Prism software.

B. Functional Results

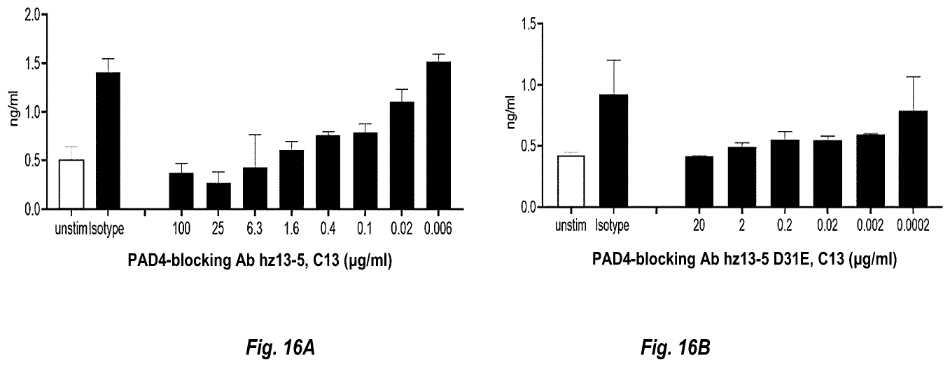

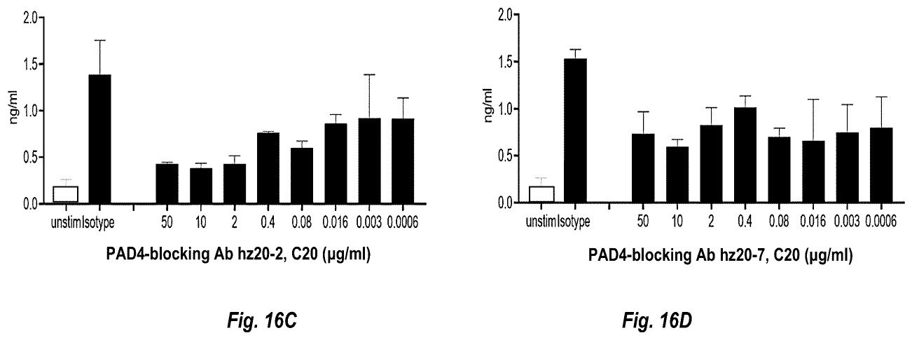

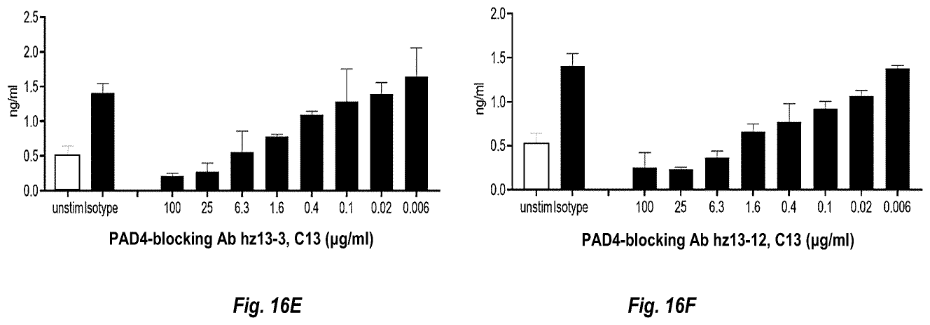

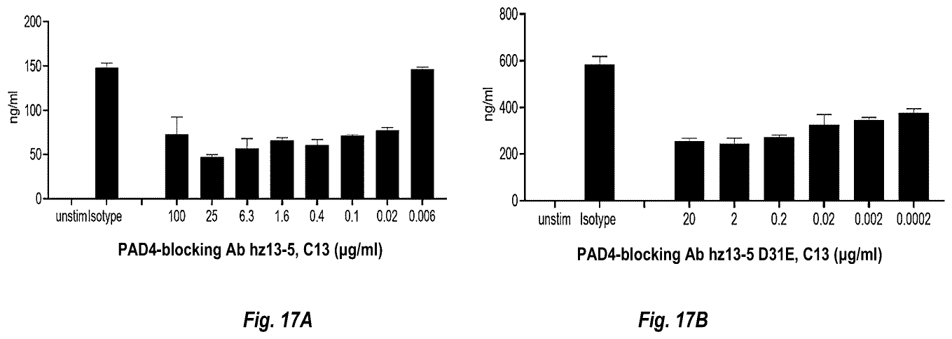

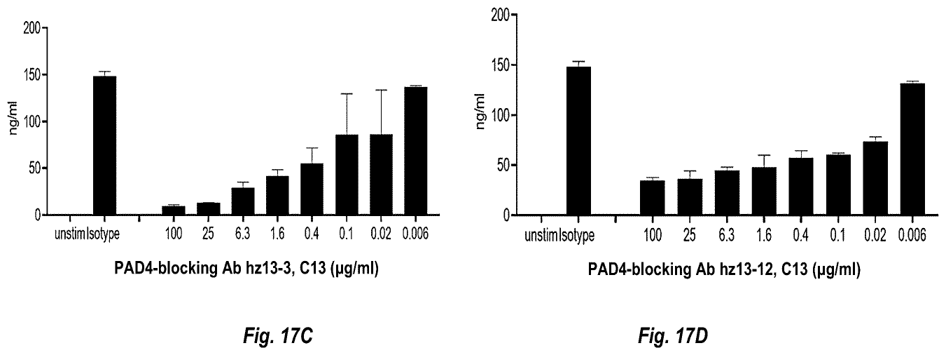

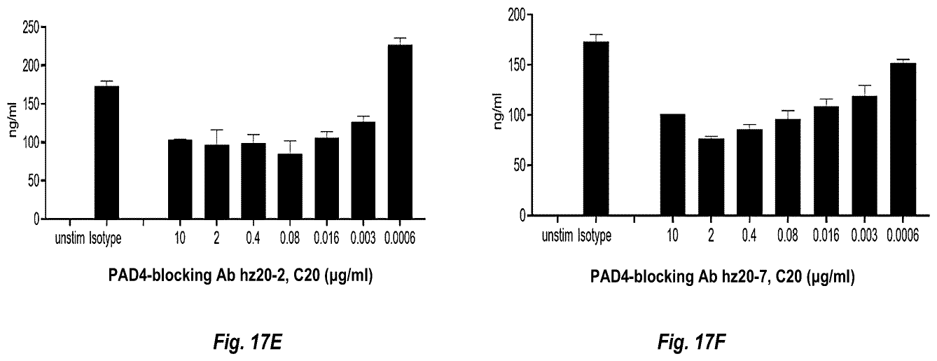

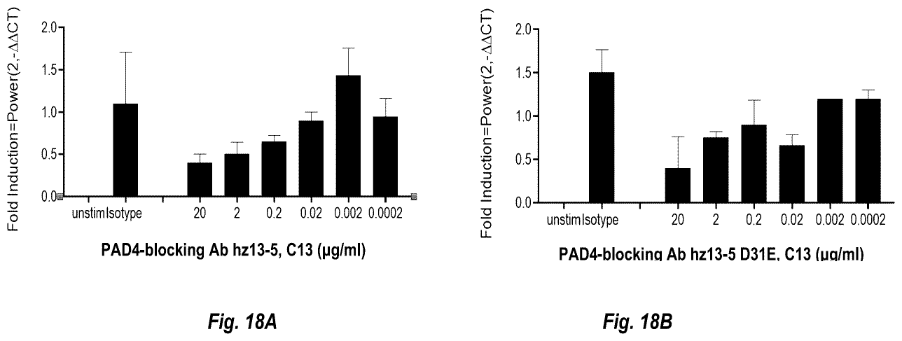

The clone 13-based antibodies reduced the amount of extracellular Cit-H3 in a dose-dependent fashion (FIG. 16A-FIG. 16F). The antibodies also reduced secretion of GM-CSF (FIG. 17A-FIG. 17F) and gene expression of GM-CSF (FIG. 18A-FIG. 18B). The antibodies also suppressed secretion and gene expression of other cytokines such as TNFα and IL-1β (data not shown).

In addition, initial imaging studies showed that anti-PAD4 antibodies can enter monocytes stimulated by LPS (see Example 18 below). The results, taken together, suggest that these antibodies can act intracellularly, resulting in suppression of the secretion and gene expression of cytokines.

### Example 18: Anti-PAD4 Antibodies Are Internalized by Monocytes

Because anti-PAD4 antibodies block GM-CSF secretion and gene expression, it is hypothesized that anti-PAD4 antibodies must enter the cell to bind PAD4. This Example describes imaging of the anti-PAD4 antibody hz13-5 D31E in monocytes, which indicates that the antibody is internalized by the monocytes.

A. Live Cell Imaging Methods

Incucyte® Live-Cell Analysis (Sartorius) was used to collect and analyze images of live cells in real time. CD14+ monocytes were isolated from healthy human whole blood containing EDTA using the EasySep™ Monocyte Isolation Kit from StemCell Technologies (Cat #19669).

Antibodies were labeled using Incucyte® Fabfluor-pH Antibody Labeling Dye (Cat #4812). The labeling dye was rehydrated with 100 μL sterile water and incubated at 37° C. for 15 minutes with the anti-PAD4 hz13-5 D31E antibody or an isotype control antibody at a 1:3 ratio in phenol-red free RPMI.

The CD14+ monocytes were suspended in Gibco phenol-red free RPMI (Cat #11835030) containing 250 nM Incucyte® Cytotox Green reagent (Cat #4633) and plated at 50 mL cells/well in Incucyte® Imagelock plates (Cat #4379). Labeled PAD4 antibody, labeled isotype control, or Fabfluor dye without antibody was added to cells. LPS (at a final concentration of 10 mM) or media containing PBS was added to each well at 15 minutes after the addition of the conjugated antibodies. The plates were placed in the Incucyte® instrument and scanned at 20× magnification every 30 minutes for 48 hours. Analysis was performed using the Incucyte® software.

B. Live Cell Imaging Results

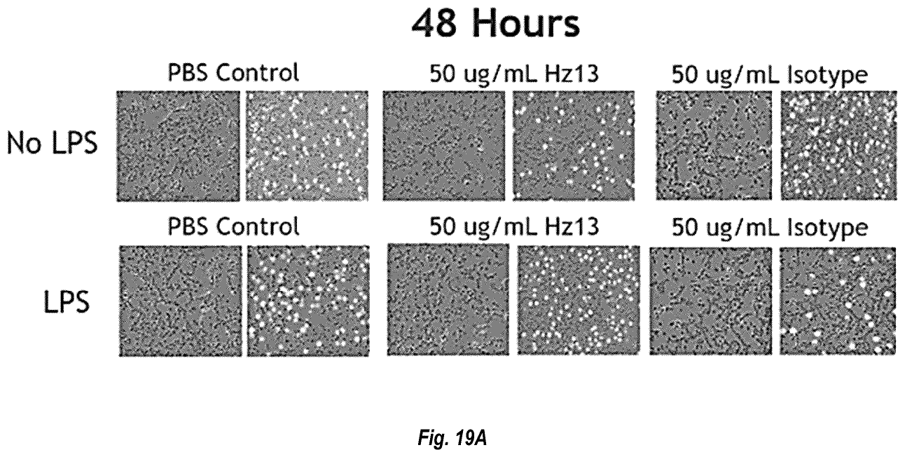

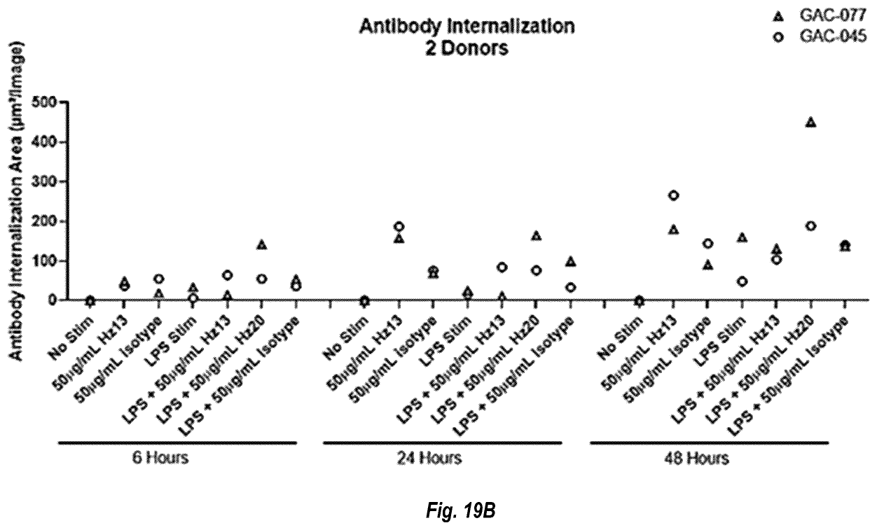

Results from live cell imaging show that the hz13-5 D31E antibody was internalized by monocytes (FIG. 19A). Hz13-5 D31E was detected in acidic cellular compartments (with a pH range of 4.5 to 5.5; e.g., late endosomes), as indicated by an increase in the appearance of the red fluorescent FabFluor®-pH dye over time (FIG. 19A). The hz13-5 D31E antibody was detected within monocytes after 6 hours (FIG. 19B). Internalization levels increased progressively at 24 and 48 hours of incubation (FIG. 19B). Antibody internalization occurred with and without LPS stimulation (FIG. 19B). The isotype control was also internalized by the monocytes but at lower levels than the PAD4 antibody (FIG. 19B).

These results indicate internalization of anti-PAD4 antibody. The results provided in this and the previous Example indicate that anti-PAD4 antibodies can act intracellularly in monocytes, resulting in suppression of cytokine gene expression and secretion. These findings indicate that anti-PAD4 antibodies can block functions of PAD4, both extracellular and intracellular.

### Example 19: Preparation of Antibodies Against Murine PAD4

A. Mouse Hybridoma Methods for Producing Anti-murine PAD4 Antibodies

The homology between human and mouse PAD4 (mPAD4) is 73%, and the anti-human PAD4 mAbs disclosed herein (Clone 20 and its derivatives as well as Clone 13 and its derivatives) do not exhibit binding to mPAD4, thus necessitating a separate campaign to identify a mouse surrogate antibody. An initial attempt was made by immunizing Balb/C mice with recombinant HIS-TVMV-mPAD4 protein produced in insect cells. After no immune response was seen, PAD4 knock-out C57bl/6 mice were used. 12 animals were immunized by 6-9 weekly injections of the same protein immunogen mixed with RIPA adjuvant. The spleens were harvested and homogenized to obtain splenocytes.

Hybridomas were generated by electro-fusion with the mouse myeloma fusion partner SP2/0-Ag14 (ATCC CRL-1581™). Fused cells were plated into multi-well plates in selective HAT medium for 6-10 days and subsequently screened for antibody secretion and binding to HIS-TVMV-mPAD4 antigen by ELISA and HTRF. These fusions yielded 467 parental hybridomas that bound specifically to mouse PAD4 (mPAD4) protein. Selected hybridoma hits were then subjected to one round of subcloning and re-assessed for antigen binding. 124 positive hybridoma sub-clones were expanded and their antibodies were purified for further characterization by SPR and a functional mouse PAD4 (mPAD4) enzymatic blocking assay.

B. Characterization Methods of Anti-Murine PAD4 Antibodies

SPR Methods. In general, SPR methods were as described in Example 3 above with the following modifications. An anti-murine capture surface was used that was prepared by immobilizing anti-murine capture antibody (Cytiva catalog #BR100838) onto flow cells of a CM5 biosensor following the manufacturer's amine coupling protocol (Cytiva catalog #BR-1006-33). SPR experiments were conducted at 37° C. using HBS-P (150 mM NaCl, 10 mM HEPES, pH 7.6, 0.05% Tween-20) (TEKNOVA catalog #H8032) with additional 500 mM NaCl and 2 mM CaCl₂) as running buffer. Several concentrations of PAD4, from 0.8 nM to 150 nM were prepared using the running buffer. The association and dissociation of the antibodies with PAD4 were measured. Two 90 second injections of 10 mM glycine pH 1.7 was used to regenerate the anti-murine capture surface.

Enzymatic Inhibition Assay. The antibodies were tested for inhibition of murine PAD4 activity against the substrate TSTGGRQGSHH (SEQ ID NO: 216) in an enzymatic blocking assay. PAD4 converts the arginine in the peptide substrate TSTGGRQGSHH to citrulline. Antibodies were tested using TSTGGRQGSHH to determine if they were able to inhibit the activity of PAD4, resulting in a decrease in the citrulline product formation. This reaction can be monitored via RapidFire™ mass spectroscopy (Agilent). In general, the materials and methods that relate to this assay are as described in Example 4 above, with the following modifications: (1) The assay conditions were as follows: 50 nM recombinant mPAD4 (Cayman Chemical, cat #28910), 500 μM TSTGGRQGSHH peptide, and 2 μl antibody solution; (2) After the reaction mixtures were prepared in a microtiter plate, the mixtures were incubated at room temperature for 90 minutes.

C. Results

More than 80 antibodies obtained from the screening were found to have K_{D} values of 100 nM or less, and 56 of these tested antibodies exhibited 80% or greater inhibition of mPAD4. One antibody, mumAb, from these 56 antibodies was selected for use in subsequent experiments described herein, and formatted as mIgG1-D265A. The functional characteristics of that selected antibody were determined using SPR and the enzymatic blocking assay described above and the results are provided in Table 12 below.

TABLE 12
Binding Kinetics of the mouse anti-mPAD4 mumAb Antibody.

| ka (1/Ms) | kd (1/s) | K_{D} (nM) | IC50 (nM) |
|---|---|---|---|
| 2.9E+05 | 2.0E−04 | 0.7 | 13.2 |

The selected mouse anti-mPAD4 antibody had the heavy chain and light chain variable region sequences and CDR sequences as set forth in the Sequence Table below.

### Example 20: The Anti-Murine PAD4 Murine Antibody Modulates PAD4 Activity In Vivo in an Acute Lung Inflammation Model

An LPS acute lung inflammation (ALI) pharmacokinetics (PK)-pharmacodynamics model was used to determine in vivo activity of the selected anti-mPAD4 murine antibody, mumAb (see Example 19) in wildtype (WT) mice with acute lung inflammation. The LPS induced lung inflammation and joint inflammation models helped determine the Pk/PD relationship of the mPAD4 and hPAD4 antibodies in joint and non joint tissues.

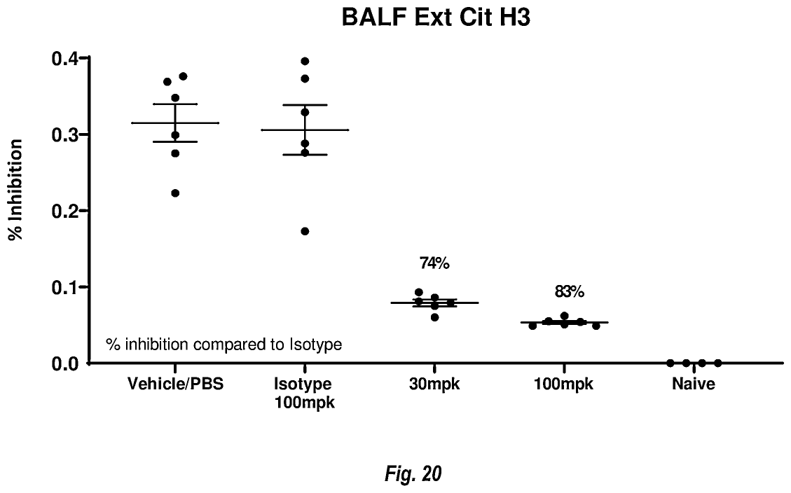

A. Methods Related to the Murine ALI Model C57Bl/6 WT mice (n=24 total) were treated with the anti-PAD4 antibody (n=6), Isotype Control antibody (IC) (n=6), or vehicle (n=6) by subcutaneous (SC) injection (day −1). The antibody was administered by sc injection at 30 mg/kg or 100 mg/kg. IC was administered at 100 mg/kg. One day after antibody/vehicle administration (i.e., on day 0), mice were nebulized with 2 mg/mL LPS (Sigma). A group of mice (n=6) was treated with PBS instead of LPS and was designated the naïve group. 48 hours after nebulization, broncheoalveolar lavage fluid (BALF) was collected from lungs. The BALF samples were centrifuged and extracellular citrullinated histone 3 (Cit-H3) in the supernatant was measured by LC/MS as a pharmacodynamics (PD) readout. % inhibition of extracellular Cit-H3 was determined by comparing the amount of extracellular Cit-H3 (expressed as the ratio of the amount of citrullinated H3 to total H3, as shown in FIG. 20) in mumAb-treated mice to the amount of extracellular Cit-H3 in IC-treated mice. Efficacy of mumAb was reflected by % reduction (% inhibition in FIG. 20) of extracellular Citrullinated Histone-3 (Cit-H3).

B. The Anti-Murine PAD4 Murine Antibody Reduces Citrullination in the LPS ALI Model

The anti-murine PAD4 mAb, mumAb, was tested for its ability to inhibit PAD4 function in the LPS ALI model as reflected by reduction of extracellular Cit-H3 in BALF (FIG. 20). When compared to BALF from mice treated with IC antibody, BALF from mice treated with 30 mg/kg mumAb had a ˜74% reduction in Cit-H3 and BALF from mice treated with 100 mg/kg mumAb had a ˜83% reduction in Cit-H3 (FIG. 20). No extracellular Cit-H3 was measured in the naïve group.

These results showed that anti-PAD4 antibody decreased citrullination of H3 in BALF and indicate that the anti-PAD4 antibody inhibits PAD4 function in the inflamed lung.

### Example 21: The Anti-Murine PAD4 Murine Antibody Modulates PAD4 Activity In Vivo in an Acute Joint Inflammation Model

An LPS acute joint inflammation (AJI) pharmacodynamics (PD) model was used to determine in vivo activity of the anti-murine PAD4 murine antibody (mumAb) in the joints of WT mice.

A. Methods Related to the Murine AJI PD Model

C57Bl/6 WT mice were treated with the anti-PAD4 antibody or Isotype Control antibody (IC) by subcutaneous (SC) injection as described in Example 20 above with the following modifications.

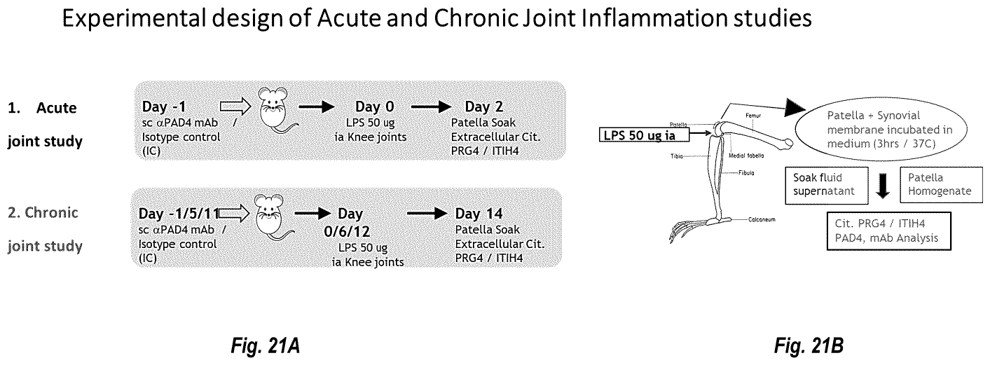

The anti-PAD4 antibody was administered at 1 mg/kg, 5 mg/kg, 220 mg/kg, or 100 mg/kg. IC was administered at 100 mg/kg. The next day after antibody/vehicle treatment (i.e., on day 0), 50 ug of LPS was administered to each mouse by intra-articular injection (ia). 48 hours later, extracellular proteins were extracted from patella and synovial membrane. Mouse patellas and synovial membranes were dissected from the knee joint and explanted with medium for 3 hrs at 37° C. These method steps are shown in FIG. 21A-FIG. 21B.

Patella explant homogenates were prepared and the supernatant from these homogenates were assayed for extracellular Cit-ITIH4 and Cit-PRG4 by LC/MS as a PD readout. The % reduction of extracellular Cit-ITIH4 and Cit-PRG4 was determined as the percentage of extracellular Cit-ITIH4 and Cit-PRG4 (expressed relative to the total amount of the relevant protein) in anti-PAD4 antibody-treated mice to that in IC-treated mice.

B. The Anti-Murine PAD4 Murine Antibody Reduces Citrullination in the LPS AJI Model

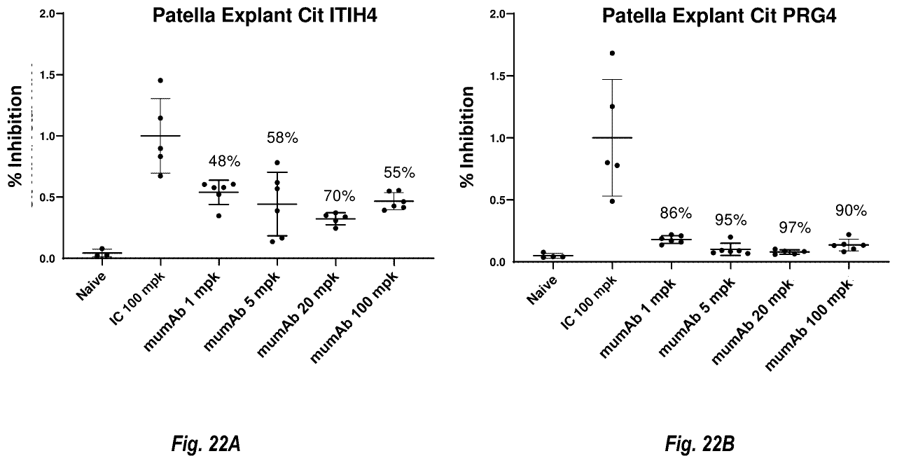

The anti-murine PAD4 mAb (mumAb) was tested for its ability to inhibit PAD4 function in the LPS AJI as reflected by reduction of extracellular Cit-ITIH4 and Cit-PRG4 in the patella explant supernatant (FIG. 22A-FIG. 22B). The mice treated with the anti-PAD4 antibody showed a reduction of up to 70% of Cit-ITIH4 (FIG. 22A) and up to 97% of Cit-PRG4 (FIG. 22B).

These results demonstrate that anti-PAD4 antibody substantially decreased citrullination of ITIH4 and PRG4 in patella explant supernatant and indicate that that the anti-PAD4 antibody inhibits PAD4 function in the acutely inflamed joint.

### Example 22: The Anti-Murine PAD4 Murine Antibody Modulates PAD4 Activity In Vivo in a Chronic Joint Inflammation Model

The LPS chronic joint inflammation (CJI) pharmacodynamics model was used to determine in vivo activity of the anti-murine PAD4 murine antibody (mumAb) in the joints of WT mice.

A. Methods Related to the Murine CJI PD Model

In general, the methods used in this Example were as described in Example 21 above with the following modifications.

Unlike the AJI model, the mice were treated with three rounds of antibody and LPS injections, in order to simulate a chronic joint inflammation condition. In each round, anti-PAD4 antibody and IC were administered by SC injection one day before an LPS ia injection (FIG. 21A). These injections were administered on days −1, 5, and 11. The next day after each treatment with anti-PAD4 antibody or IC, 50 ug of LPS was administered by ia injection (i.e., on days 0, 6, and 12 (FIG. 21A-FIG. 21B). 48 hrs after the third LPS ia injection, patella explant homogenates were prepared and the supernatant from these homogenates was assayed for extracellular Cit-ITIH4 and Cit-PRG4.

B. The Anti-Murine PAD4 Murine Antibody Reduces Citrullination in the LPS CJI PD Model

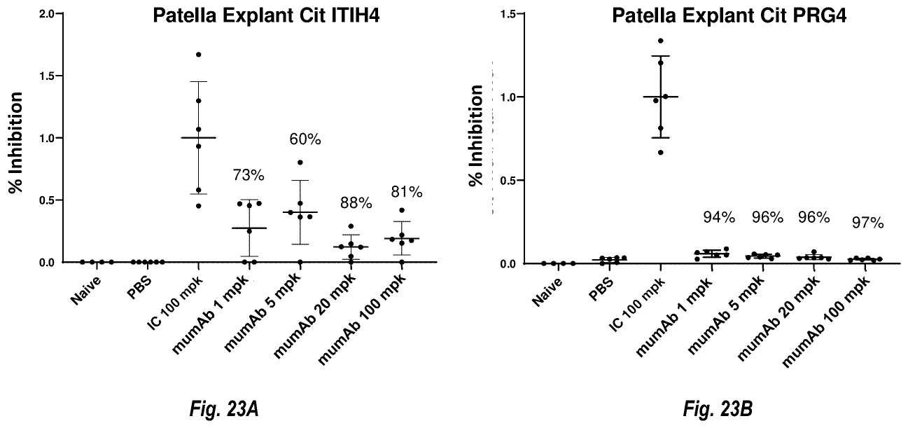

The anti-PAD4 antibody was tested for its ability to inhibit PAD4 function in the LPS CJI model as reflected by reduction of extracellular Cit-ITIH4 and Cit-PRG4 in the patella explant supernatant (FIG. 23A-FIG. 23B). The mice treated with the anti-PAD4 antibody showed a reduction of up to 88% of Cit-ITIH4 (FIG. 23A) and up to 97% of Cit-PRRG4 (FIG. 23B). Samples from mice treated with 1 mg/kg or more of the anti-PAD4 antibody showed a reduction of Cit-PRG4 of 94% or more (FIG. 23B).

These results demonstrate that anti-PAD4 antibody substantially decreased citrullination of ITIH4 and PRG4 in patella explant supernatant and indicate that the anti-PAD4 antibody inhibits PAD4 function in the chronically inflamed joint.

### Example 23: The Anti-Murine PAD4 Murine Antibody Modulates PAD4 Activity In Vivo in a Pristane-Induced Peritonitis Model

Anti-murine PAD4 mAb, mumAb, was tested for its ability to inhibit several PAD4 dependent responses in vivo in a short term pharmacodynamics (PD) model.

A. Materials and Methods for Mouse Treatments and Pristane Challenge

The following materials and instruments were used in the study described in this Example. (1) Pristane; Sigma; Cat #P2870-100 ml. (2) BALB/c mice; female; 10 to 12 weeks of age. (3) Anti-PAD4 ab mumAb. (4) Isotype Control antibody (IC). (5) 1×PBS with 4 mM EDTA pH 7.4; Teknova; Cat #P0203. (6) ACK Lysis Buffer; Gibco; Cat #A10492-01 100 ml. (7) MACS Buffer; Macs Militenyi Biotec; Cat #130-091-221. (8) Standard FACS antibodies. (9) Luna-II Automated Cell Counter; Logos Biosystems. (10) 96-well U bottom plates. (11) Fetal Calf Serum (FCS); Summit; Cat #S-100-050; (12) DPBS; Gibco; Cat #14190 (13) FACS tubes; BD Biosciences; Cat #352063. (14) 40-04 Tissue Filters; BD Falcon; Cat #352350. (15) GentleMACS C TUBES; Millitenyi; Cat #130-096-334. (16) Fixation and permeabilization solution; BD Biosciences; Cat #554722. (17) Fc Block; ebiosciences; Cat #14-0161-86. (17) Multi tissue dissociation kit; Miltenyi; cat #130-110-203. (19) AF647 conjugation kit; abcam; Cat #AB269823. (19) AF488 conjugation kit; abcam.

BALB/c female mice of 10-12 weeks of age were used. The mice were randomized based on body weight into 5 treatment groups as shown in Table 13 below.

TABLE 13
Treatment Groups of BALB/c Mice.

| Group | | |
|---|---|---|
| Number | n | Description |
| I | 6 | Naïve group |
| II | 6 | treated with Isotype Control (IC) 100 mg/kg via sc injection |
| III | 6 | treated with mumAb; 10 mg/kg via sc injection |
| IV | 6 | treated with mumAb; 30 mg/kg via sc injection |
| V | 6 | treated with mumAb; 100 mg/kg via sc injection |

MumAb was administered to mice in groups III, IV, and V at 10 mg/kg, 30 mg/kg, and 100 mg/kg, respectively. Treatment was administered on day −1, i.e., 24 hrs before Pristane challenge). MumAb was prepared in sterile PBS. Group II mice were treated with IC at 100 mg/kg. The dosing volume for all treatments was 10 mL per kg of body weight.

Pristane was administered to mice (i.e., the Pristane Challenge) via intraperitoneal (ip) injection. 500 μl of Pristane was administered to mice from Groups II to V on day 0, i.e., 24 hrs after treatment with mumAb. Group I mice received ip injection of saline.

16 hrs after Pristane Challenge, mice were anesthetized, and plasma samples were collected. The mice were also euthanized, and 2 mL of PBS-EDTA solution was administered by ip injection. The abdomen of the mice were massaged for 20-30 seconds. Then, 2 mL of the injected PBS-EDTA solution was aspirated along with peritoneal fluid and cells using 2 mL syringe.

Peritoneal fluid and cells were transferred to centrifuge tubes and kept on ice until processing. Peritoneal fluid and cell samples were centrifuged at 1500 rpm and 4° C. for 5 mins. The supernatant from the samples were stored at −80° C. until further analysis for extracellular Cit-H3, extracellular PAD4, myeloperoxidase (MPO) and neutrophil elastase (NE) by ELISA. Cytokine and chemokine levels in plasma and peritoneal fluid samples were measured using a Luminex® assay.

In some instances, peritoneal cell pellets were lysed with red blood cell (RBC) lysis buffer, if necessary, washed in MACS buffer, and centrifuged at 1500 rpm and 4° C. for 5 mins. Peritoneal cell counts were recorded using a cell counter, such as Luna-II Automated Cell counter. In some instances, peritoneal cell pellets were resuspended in MACS buffer and divided into separate panels for staining for NETois/METosis and citrullination.

B. NETosis/METosis and Citrullination Staining Protocol and Materials

MEtosis is the process by which extracellular traps composed of cellular DNA studded with histones and cellular proteins are released from monocytes or macrophages. This net-like material formed by either METosis or NETosis (neutrophil origin) is important in defense against microbes but is also a primary driver of autoimmune pathology and aseptic inflammation. NETosis was assessed by Sytox Green™ (SG), myeloperoxidase (MPO), and neutrophils. METosis was assessed by SG, MPO, and monocytes/macrophages.

Staining Procedure for SG/MPO (Panel 1—Surface Panel—NETosis/METosis) was as follows.

TABLE 14
Flow Panel 1.

| Flurorophore | Markers | Cat # |
|---|---|---|
| FITC | Sytox Green | S7020, Thermo Fisher |
| Unconjugated | MPO | A039829-2, Agilent Dako |
| R-Phycoerythrin AffiniPure | Secondary | 711-116-152, Jackson |
| F(ab′)₂ Fragment Donkey | Antibody | ImmunoResearch |
| Anti-Rabbit IgG (H + L) (PE) | | Laboratories Inc. |
| APC CY7 | Ly6G | 127624, Biolegend |
| AF700 | CD45 | 103128, Biolegend |
| BV421 | CD11B | 101236, Biolegend |
| BV510 | ZA | 423102, Biolegend |
| BV605 | Ly6C | 128036, Biolegend |
| Pecy7 | F4/80 | 123114, Biolegend |

The cells were incubated with the Fc block for 15 mins on ice (1 μL of mouse Fc block per sample) and were stained with 50 μL of surface staining antibody cocktail prepared in MACS buffer for 15 mins on ice.

A Sytox Green™ working solution was prepared by making a 1:3000 dilution of Sytox green stock solution made to achieve a concentration of 1.667 μM.

Zombie™ aqua cocktail was prepared by adding 100 μL of DMSO to a vial of Zombie™ aqua. This solution was used for live/dead staining of cells.

50 μL of Sytox™ green and 50 μL of Zombie™ aqua cocktail was added and the mixture was further incubated for 15 mins on ice. The stained samples were centrifuged at 1500 rpm and 4° C. for 5 mins. The plates were washed twice with MACS buffer.

100 μL of diluted Cytofix™ (1 part Cytofix™ and 4 parts MACS buffer) was added, mixed, and incubated for 15 mins on ice. The cells were washed twice with MACS buffer and stored in the plate at 4° C. overnight.

The next day, the cells were washed with MACS buffer. 100 μL of PE secondary antibody (at a 1:1000 dilution in MACS buffer) was added to each sample and the samples were incubated for 30 mins on ice.

The samples were washed twice with Cytofix buffer (i.e., 1 part Cytofix and 4 parts MACS buffer). Cells were reconstituted in 200 μL of MACS buffer. All samples were analyzed using the FACSCanto™ Flow Cytometry System.

Staining Procedure for PAD4/Cit-H3: Panel 2—Intracellular Panel—Citrullination.

TABLE 15
Flow Panel 2.

| Flurorophore | Markers | Cat # |
|---|---|---|
| FITC | PAD4-3D1 | In-house reagent |
| AF647 | Cit-H3 | ab5103 |
| APC CY7 | Ly6G | 127624, Biolegend |
| BV421 | CD11B | 101236, Biolegend |
| BV510 | ZA | 423102, Biolegend |
| BV605 | Ly6C | 128036, Biolegend |
| AF700 | CD45 | 103128, Biolegend |
| Pecy7 | F4/80 | 123114, Biolegend |
| PerCP | CD206 | |

The cells were incubated with the Fc block for 15 mins on ice and the cells were stained with 50 μL of surface staining antibody cocktail for 15 mins on ice. 50 μL of Zombie™ aqua cocktail was added and the samples were further incubated for 15 mins on ice. The samples were washed twice at 1500 rpm and 4° C. for 5 mins.

150 μL of Cytofix™/cytoperm was added to each sample. The samples were mixed and incubated for 20 mins on ice. The cells were washed twice with MACS buffer and stored in the plate at 4° C. overnight.

Antibody Conjugation Methods. For conjugation of PAD4, 100 μg of PAD4-3D1 protein in DPBS was conjugated with AF488 using the AF488 conjugation kit. The conjugation reaction was carried out overnight. For conjugation of Cit-H3, 100 μg of Cit-H3 in DPBS was conjugated with AF647 using the AF647 conjugation kit. The conjugation reaction was carried out overnight.

The cells were permeabilized with 200 μL of 1× permeabilization buffer from the AF488 conjugation kit for 20-30 mins on ice. The cells were stained with 50 μL of intracellular cocktail mix (with PAD4-F488, Cit H3-AF647, and CD206) in 1× permeabilization buffer for 30 mins on ice.

The samples were washed twice with MACS buffer and centrifuged at 1500 rpm and 4° C. for 5 mins. The cells were reconstituted in 200 μL MACS buffer.

All samples were analyzed using the FACSCanto™ Flow Cytometry System and the data were analyzed using FlowJo® v.10.

C. Results

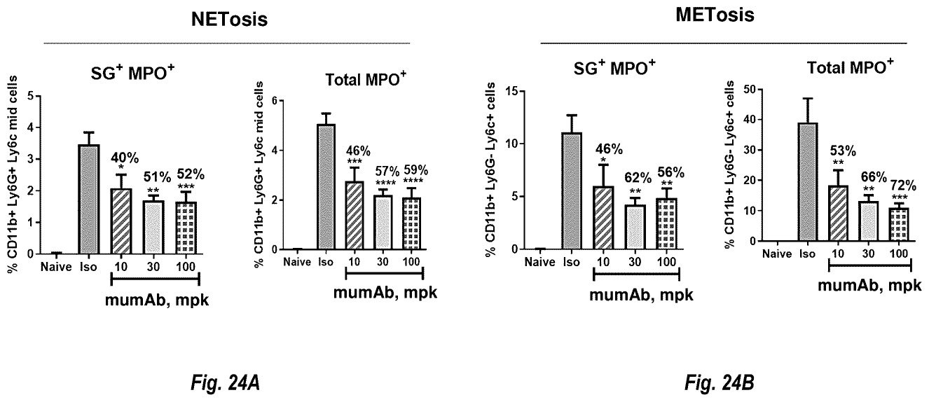

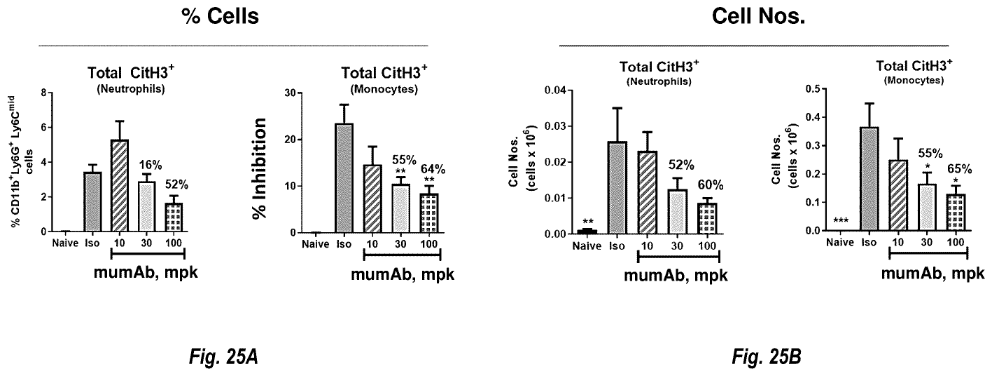

As shown in FIG. 24A-FIG. 24B, Anti-PAD4 mAb mumAb, significantly reduced pristane-induced extracellular trap forming neutrophils (NETosis) and extracellular trap forming monocytes (METosis). Similarly, mumAb treatment resulted in significantly reduced proportions of total Cit-H3+ neutrophils and monocytes (FIG. 25A) as well as significantly reduced numbers of Cit-H3+ neutrophils and monocytes (FIG. 25B). In M1 macrophages and M2 macrophages, mumAb reduced the total Cit-H3+ cells (FIG. 26A and FIG. 26B, left graphs) and the MFI of CitH3 (FIG. 26A and FIG. 26B, right graphs); the effects observed were greater in M2 macrophages (cf. FIG. 26A (M1 macrophage results) and FIG. 26B (M2 macrophage results)). The results indicate that treatment with Anti-PAD4 mAb decreased inflammation.

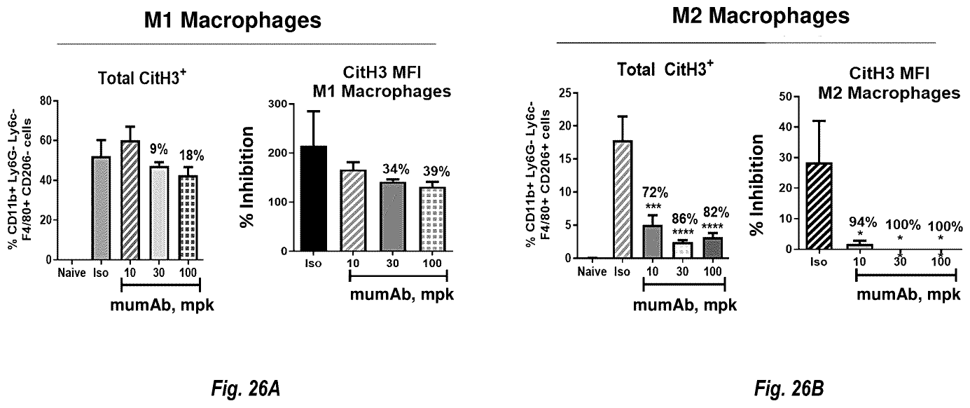

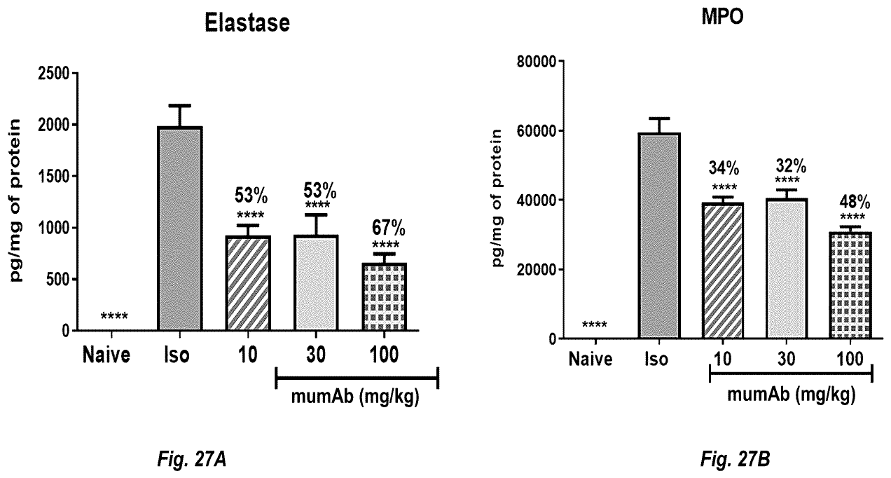

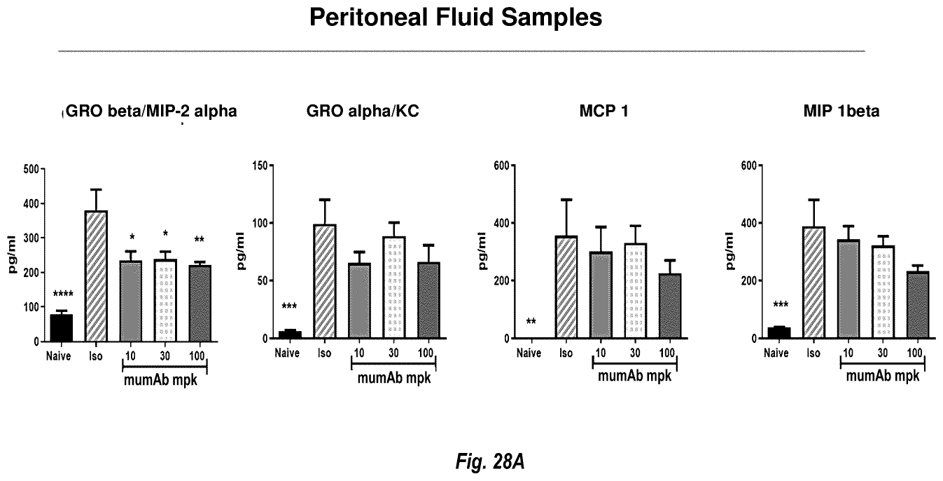

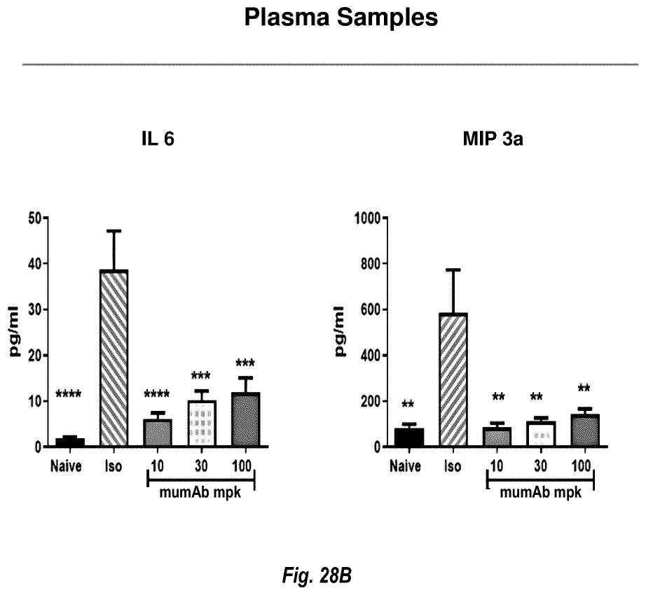

Because elastase serves as a soluble marker for neutrophil NETosis and MPO represents a soluble marker of both neutrophil NETosis and monocytes/macrophage METosis, the impact of mumAb on these markers was evaluated in peritoneal fluid. Treatment of mice with different doses of mumAb significantly reduced both elastase (FIG. 27A) and MPO (FIG. 27B) in peritoneal fluid obtained from pristane-treated mice. MumAb also reduced chemokines MIP-2 alpha, GRO alpha/KC, MCP1 and MIP 1beta in peritoneal fluid samples, and the reduction of MIP-2 alpha was statistically significant. MumAb also significantly reduced cytokine IL6 (FIG. 28B, left graph) and chemokine MIP3 alpha (FIG. 28B, right graph) in plasma samples.

### Example 24: The Anti-Murine PAD4 Murine Antibody Modulates PAD4 Activity In Vivo in a Collagen-Induced Arthritis (CIA) Model

Anti-PAD4 mAb, mumAb, was tested for its ability to inhibit disease severity and several PAD4 dependent responses in a collagen-induced arthritis (CIA) model with both preventative and semi-therapeutic dose regimens.

A. Materials and Methods for Mouse Treatments

The materials and instruments used in this Example were as described in Example 23 above with a few modifications. In this Example, pristane was not used. Also, male DBA1 mice (Envigo USA) at 13 to 14 weeks of age were used instead of BALB/c female mice.

A collagen solution was prepared as follows. 0.05 M acetic acid was prepared by adding 30 μL of glacial acetic acid into 9.97 mL of ice cold MilliQ Water. 2.5 mL of 0.05 M acetic acid was added to a vial containing 10 mg of bovine type II collagen (Chondrex; Cat #20021). The mixture was stored overnight at 4° C. for dissolution.

A collagen emulsion was prepared as follows. Vials of the Sigma Adjuvant System (Sigma Aldrich; Cat #S6322) and saline solution was incubated at 37° C. to 40° C. for 10-15 min. 0.5 mL of saline was added into each vial of the Sigma Adjuvant System through the septum using a 1.0 mL syringe. Each vial was vortexed vial thoroughly for at least 2 min. the vials were incubated for another 15 min at 37° C. to 40° C. 0.5 mL of collagen solution was added to each vial and the mixture was vortexed for 1-2 mins.

Male DBA1 mice used this study were divided into 6 different groups. The mice were acclimatized for at least one week in the holding area prior to initiation of experimentation.

TABLE 16
Treatment Groups of DBA1 Mice.

| Group | | |
|---|---|---|
| Number | n | Description |
| I | 5 | Naïve group |
| II | 15 | treated with Isotype Control (IC) 100 mg/kg via sc injection |
| | | 1x/week |
| III | 15 | treated with mumAb; 10 mg/kg via sc injection; 1x/week |
| IV | 15 | treated with mumAb; 30 mg/kg via sc injection; 1x/week |
| V | 15 | treated with mumAb; 100 mg/kg via sc injection; 1x/week |
| VI | 10 | Treated with CTLA4-Ig; 3 mg/kg via ip injection; 2x/week |

Two separate studies were performed—(1) a preventative study and (2) a semi-therapeutic study. Both studies share the same primary immunization protocol. On the day of collagen immunization (day 0), a collagen emulsion is prepared as described above. The DBA1 mice were anesthetized using isoflurane. The tail of each mouse was disinfected with 70% isopropyl alcohol and 100 μL of emulsion was slow and firmly administered via sc injection in 2 regions about 1.0-2.0 cm from the base of the tail.

For the preventative study, the mice were treated on day 0. Mice were immunized with bovine type II collagen. Starting on day 0, the mice were treated with 10 mg/kg, 30 mg/kg and 100 mg/kg of mumAb in sterile PBS. The different mumAb doses were administered via sc injection once per week to groups III, IV, and V on Day 0 (Table 16). Mice in Group II were treated with IC (100 mg/kg) by sc injection (Table 16). CTLA4-Ig was administered by ip injection twice a week (Table 16). A booster dose of bovine type II collagen was administered on day 21. Clinical scores and disease incidences were measured weekly.

For the semi-therapeutic study, the mice were treated as described in the preventative study above, with the following modifications. The mice were treated with 10 mg/kg, 30 mg/kg and 100 mg/kg of mumAb starting on day 21 instead of day 0.

All mice were observed daily for any abnormal behavior. Symptoms (i.e., clinical scores) were assessed twice weekly along with body weight. Plasma and/or serum samples were collected at different time points to determine the concentration of the mumAb for the preventative and semi-therapeutic studies.

Both studies were terminated on day 44 and the mice were euthanized. Clinical scores and paw weights on day 44 were captured. Mice were bled for plasma and/or serum and stored at −80° C. for various assays. Tissue samples (from paw and patella) were collected and immediately processed to produce single cell suspensions for flow cytometry. Paw samples were also collected for histology, ELISA, and other assays.

B. Scoring Method for Arthritis

Arthritis was scored according to the following scale: (1) normal; (2) mild, with definite redness and swelling of the ankle or wrist, or with apparent redness and swelling limited to individual digits, regardless of the number of affected digits; (3) moderate redness and swelling of ankle or wrist; (4) severe redness and swelling of the entire paw including digits; (5) maximally inflamed limb with involvement of multiple joints.

C. Methods for Processing Paw Samples for Flow Cytometry

Mice were euthanized at the end of the preventative and semi-therapeutic studies. Inflamed hind paw was severed (from tibiotarsal joints), collected in RPMI, and kept on ice. The skin was removed from the inflamed hind paw sample. Terminal end of the tibia-fibula, tarsus and meta tarsus bones was dissected. Digits were excluded from processing. The inflamed joint and muscle tissue were divided into pieces and were collected in MACS C tubes. The samples were digested with Multi Tissue Dissociation Kit 2 (Miltenyi; Cat #130-110-203) according to manufacturer's protocol. The total volume per sample was 5 mL.

The pMACS octo dissociator was used for tissue digestion at 37 C. The samples were passed through a 40-μm sieve and were collected in a 50-mL tube. The final volume for each sample was made up to 25 mL with 1×DPBS (Thermo Fisher; Cat #14190235). The samples were centrifuged at 1500 rpm and 4° C. for 5 mins.

1 mL of ACK buffer (Thermo Fisher; Cat #A1049201) was added to the pellet for each sample, mixed well, incubated at room temperature for 1 min for RBC lysis. The volume for each sample was made up to 15 mL with 1×DPBS. The samples were centrifuged again at 1500 rpm and 4° C. for 5 mins.

Each sample pellet was reconstituted in 0.5 mL of MACS running buffer (Miltenyi; Cat #130-091-221). The cells from the pellets were stained for histological analysis and processed according to protocols for NETois/METosis and citrullination staining described below.

D. NETosis/METosis and Citrullination Staining Protocol and Materials

Staining procedure for SG/MPO: Panel 1—Surface Panel—NETosis/METosis was as described above in Example 23.

Staining procedure for PAD4/Cit-H3: Panel 2—Intracellular Panel—Citrullination was as described above in Example 23.

Antibody conjugation methods were as described above in Example 23.

E. Histology Staining Methods

Paw samples were stained with hematoxylin and eosin and subjected to histological analysis.

F. Methods for Processing Mouse Hind Paw/Joints for Quantifying Anti Cyclical Citrullinated Peptide Antibody (ACPA) and ELISA

Mice were euthanized at the end of the preventative and semi-therapeutic studies. Inflamed hind paw was severed (from tibiotarsal joints) and collected in RPMI, kept on ice. Paw samples were finely divided with scissors and the finely divided tissue samples were collected in 2-mL tubes. 1 mL of 1×RIPA buffer (Cell Signaling; Cat #9806) was added to each 2 mL tube. A metal ball was also added per tube. The 2-mL samples were placed in Tissue Lyser II (Qiagen) for bead homogenization.

After homogenization, the samples were centrifuged at 15000×g and 4° C. for 20 mins. The supernatant was collected from each sample and subsequently analyzed for (1) anti cyclical citrullinated peptide antibody (ACPA; MyBioSource.com; Cat #MBS2607007), (2) PAD4 (Cusabio; Cat #CSBFL017379M0), (3) MPO (R&D Systems; Cat #DY3667), and (4) neutrophil elastase EA2 (R&D Systems; Cat #DY4517-05) by ELISA.

G. Results of the Preventative Study

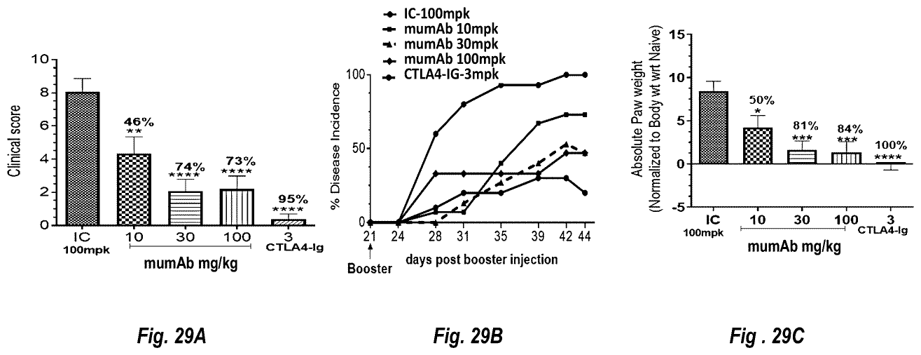

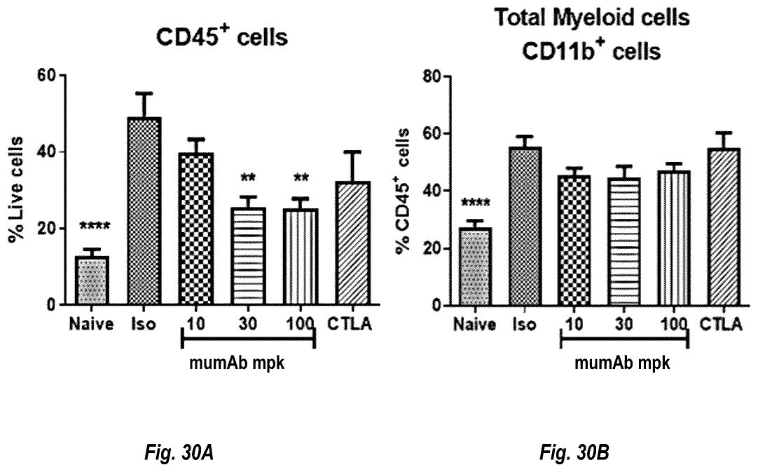

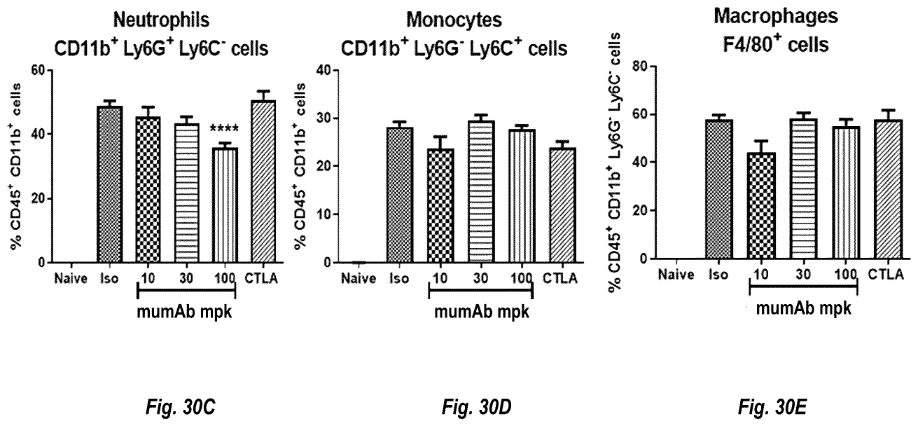

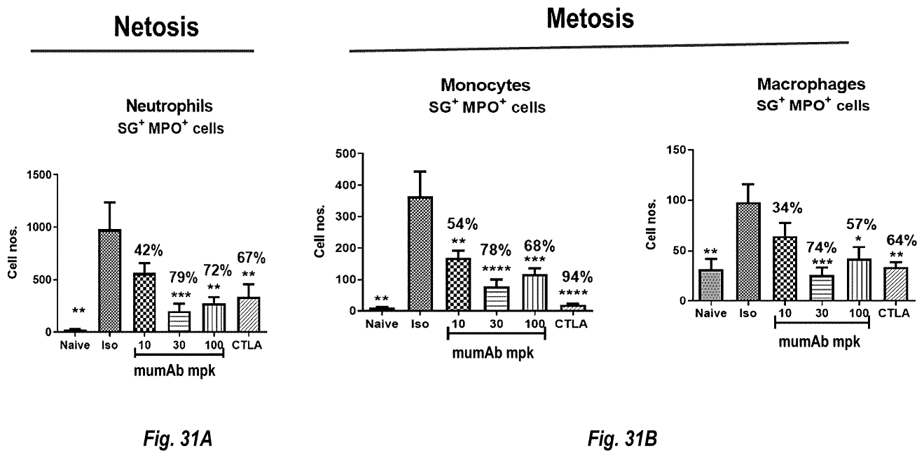

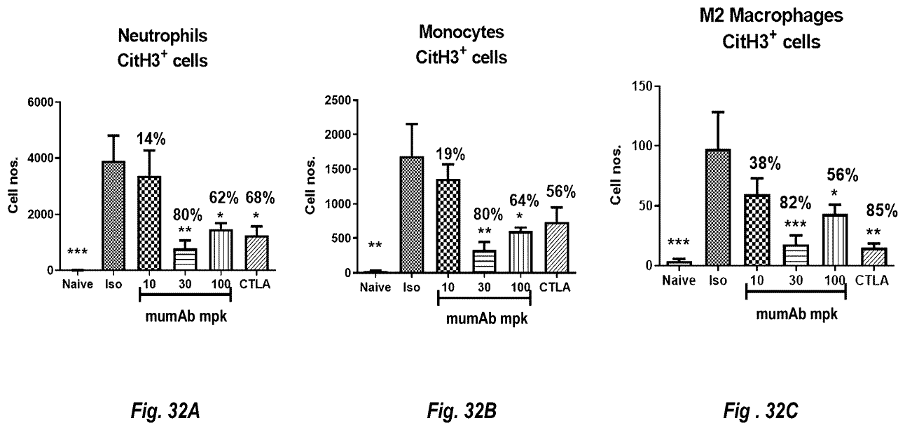

As shown in FIG. 29A, Anti-PAD4 mAb, mumAb, significantly reduced arthritis clinical score. Disease incidences and paw weights were also significantly reduced (FIG. 29B and FIG. 29C). MumAb also reduced infiltration of immune cells (FIG. 30A-FIG. 30E). As shown in FIG. 31A-FIG. 31B, mumAb significantly reduced NETosis and METosis as assessed by SG+ MPO+ neutrophils, and SG+ MPO+ monocytes/macrophages, respectively. Similarly, mumAb showed significant reduction in proportion of total Cit-H3+ neutrophils, monocytes and macrophages (FIG. 32A-FIG. 32C).

Because elastase and MPO represent soluble markers of neutrophils and monocytes/macrophages, the impact of mumAb on these markers was evaluated in in paw homogenate. Treatment of mice with different doses of mumAb significantly reduced both MPO and elastase (FIG. 33B-FIG. 33C). PAD4 levels were also significantly reduced (FIG. 33A).

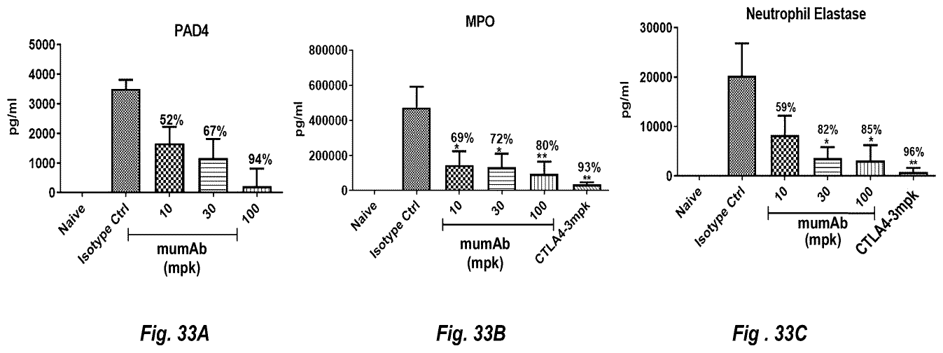

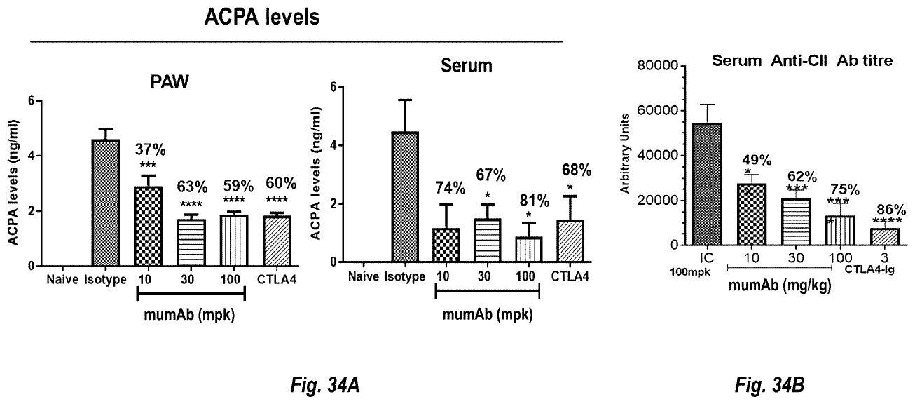

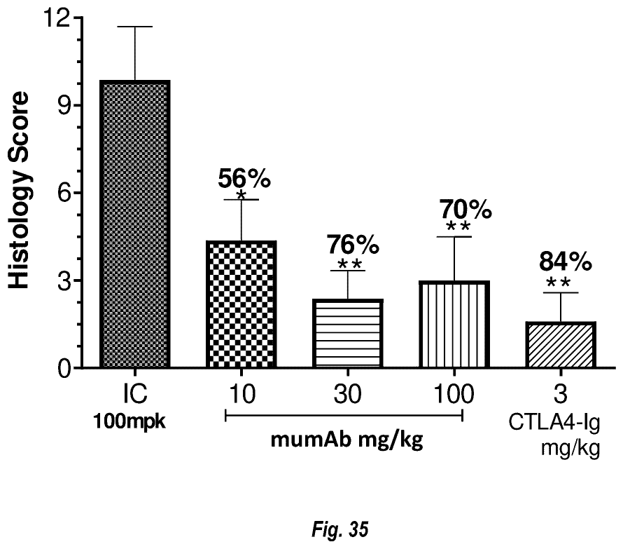

Additionally, paw homogenate and serum samples were analyzed for ACPA detection by ELISA. MumAb showed significant reduction of ACPA in both paw homogenate and serum (FIG. 34A). Similarly, titer of anti-CII Ab was also significantly reduced (FIG. 34B). MumAb also showed significant reduction in paw histology score (FIG. 35).

H. Results of the Semi-Therapeutic Study

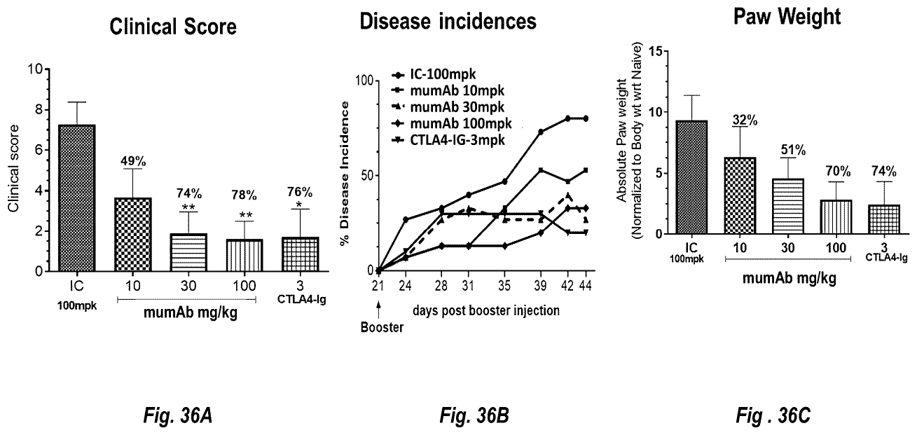

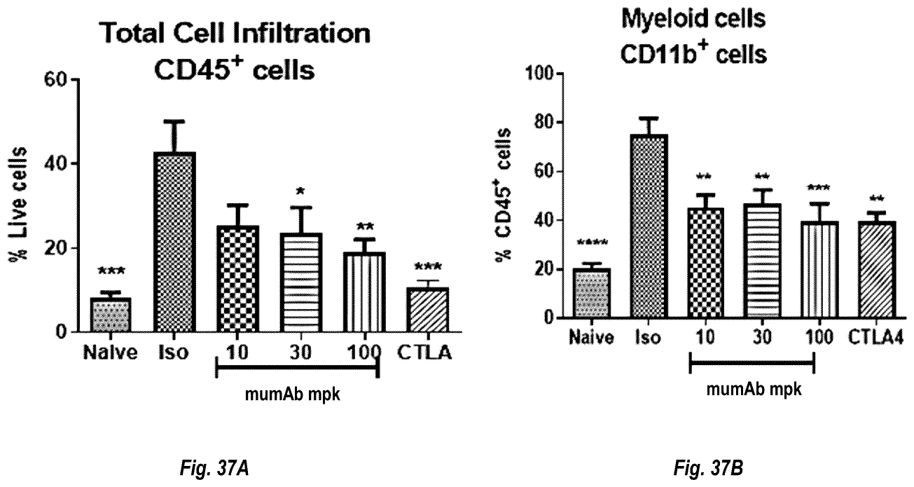

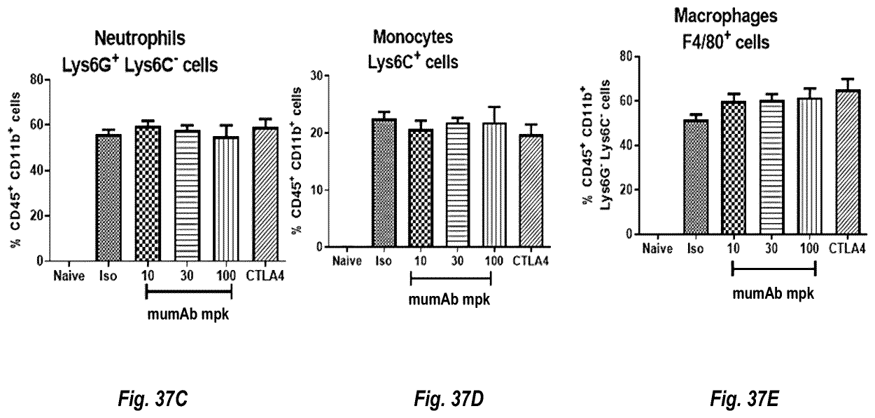

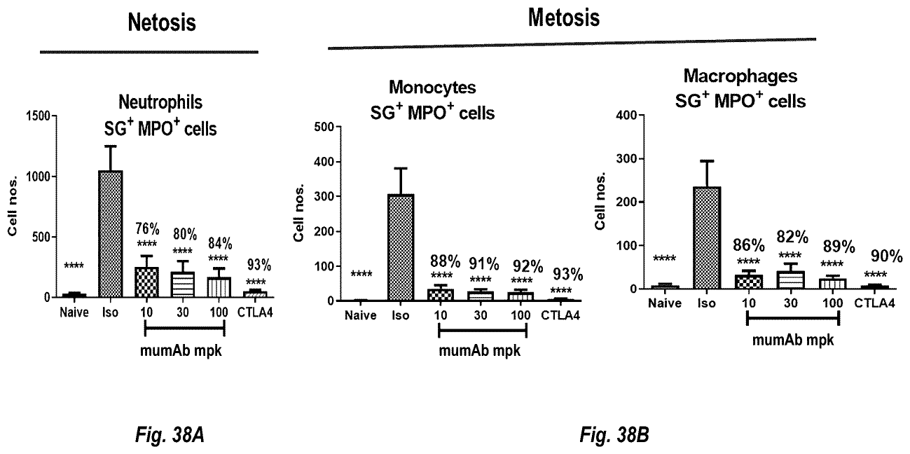

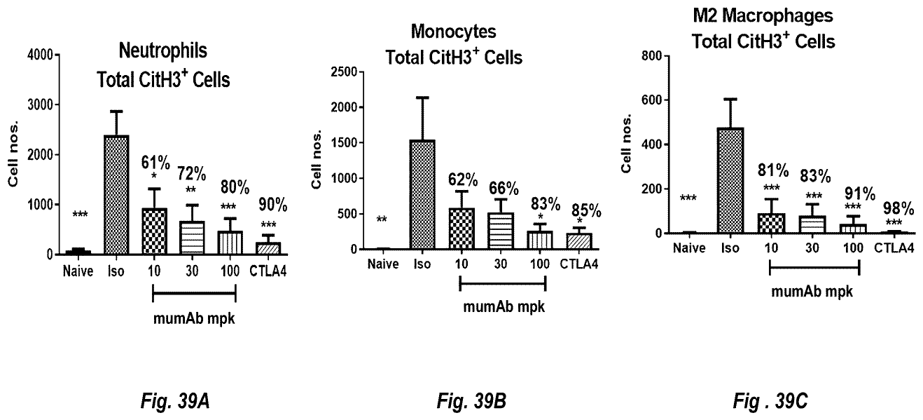

As shown in FIG. 36A, mumAb significantly reduced arthritis clinical score. Disease incidences and paw weights were also significantly reduced (FIG. 36B-FIG. 36C). MumAb also reduced infiltration of total immune cells and myeloid cells (FIG. 37A-FIG. 37E). Furthermore, mumAb significantly reduced NETosis as assessed by SG+ MPO+ neutrophils (FIG. 38A) and also significantly reduced METosis as indicated by monocytes and macrophages (FIG. 38B). Similarly, mumAb significantly reduced the total Cit-H3+ neutrophils, monocytes and M2 macrophages (FIG. 39A-FIG. 39C, respectively).

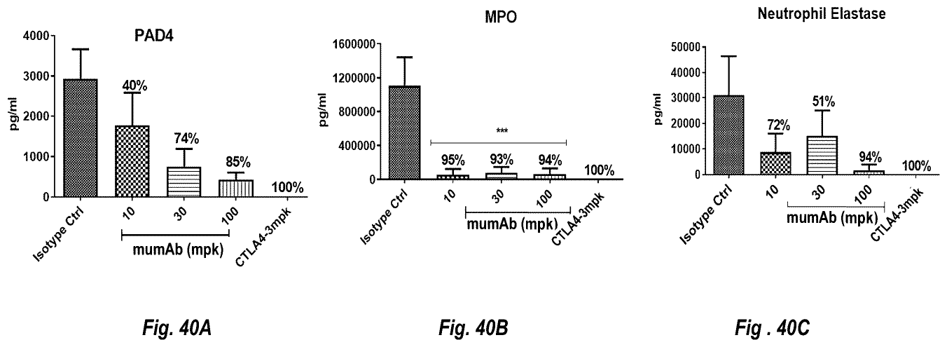

Because elastase and MPO represent soluble markers of neutrophils and monocytes/macrophages, the impact of mumAb on these markers was evaluated in in paw homogenate. Treatment of mice with different doses of mumAb significantly reduced both MPO and elastase (FIG. 40B-FIG. 40C). PAD4 levels were also significantly reduced (FIG. 40A).

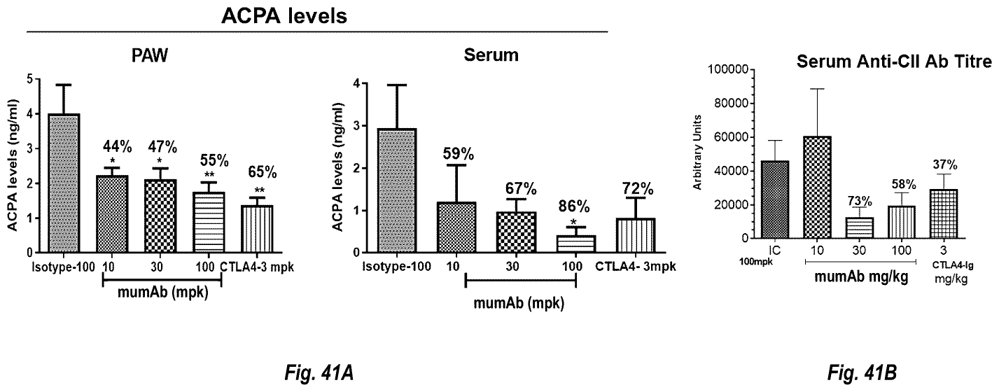

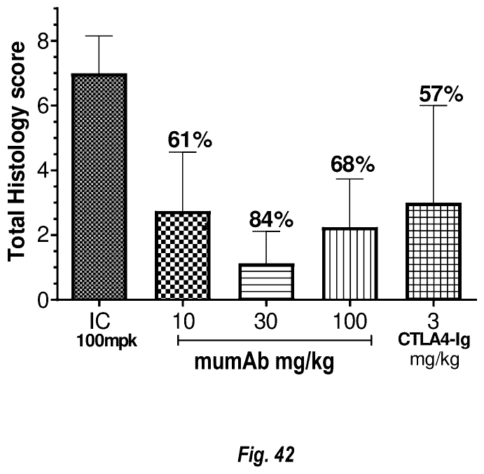

Additionally, paw homogenate and serum samples were analyzed for ACPA detection by ELISA. MumAb showed significant reduction of ACPA in both paw homogenate and serum (FIG. 41A). Similarly, titer of anti-CII Ab was also significantly reduced (FIG. 41B). MumAb also showed significant reduction in paw histology score (FIG. 42).

### Example 25: Activity of Anti-Human PAD4 Antibodies in an LPS-Induced Acute Lung Inflammation Model

The LPS ALI PD model was used to determine the in vivo activity of anti-human PAD4 antibodies (Ab) in human PAD4 knock-in (HU-PAD4KI) mice.

A. Methods Related to the Murine ALI PD Model

The methods related to the murine ALI PD model in this Example were as described in Example 20 above with the following modifications. C57Bl/6 human-PAD4 knock-in (Hu-PAD4 KI) mice were used as well as WT mice. These mice were treated with antibodies shown in Table 17 below at 0.24 mg/kg, 1.2 mg/kg, 6 mg/kg, 3 mg/kg, 10 mg/kg, 30 mg/kg, and/or 100 mg/kg. Control mice were treated with Isotype control murine antibody (IC mAb) at 100 mg/kg.

B. Results

Anti-human PAD4 mAbs clone 13 (mAb13) and clone 20 (mAb20) were tested for their ability to inhibit PAD4 function in the LPS ALI PD model. The humanized derivatives of clone 13, (1) hz13-5 and (2) hz13-12, and the humanized derivatives of clone 20, (1) hz20-2 and (2) hz20-7, were also tested. The activity of the antibodies was assessed based on their inhibition of the level of extracellular citrullinated H3 (relative to total extracellular H3) and the level of extracellular citrullinated ITIH4 (relative to total extracellular ITIH4). Compared with isotype control, all of these antibodies reduced extracellular Cit-H3 and extracellular Cit-ITIH4 in the Hu-PAD4 KI mice.

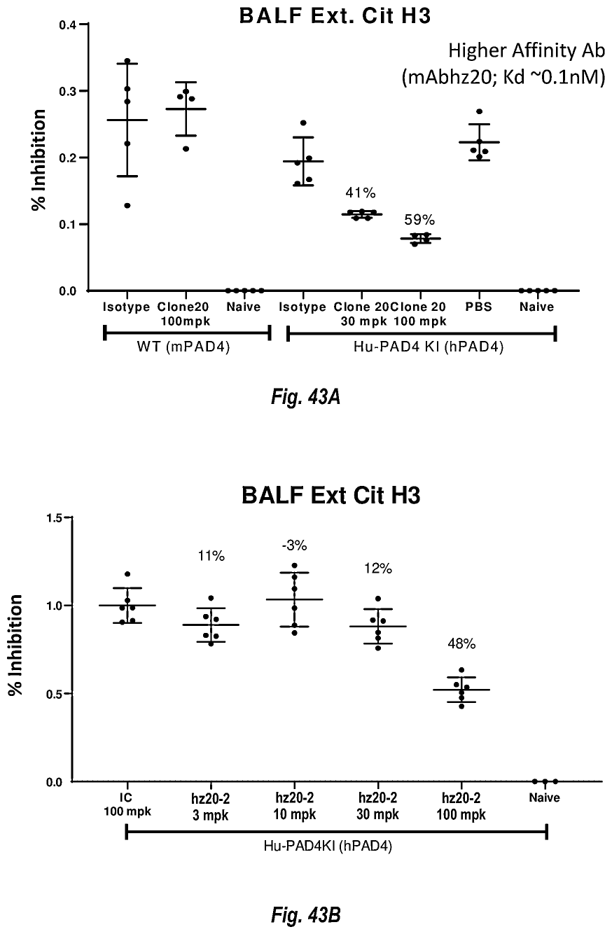

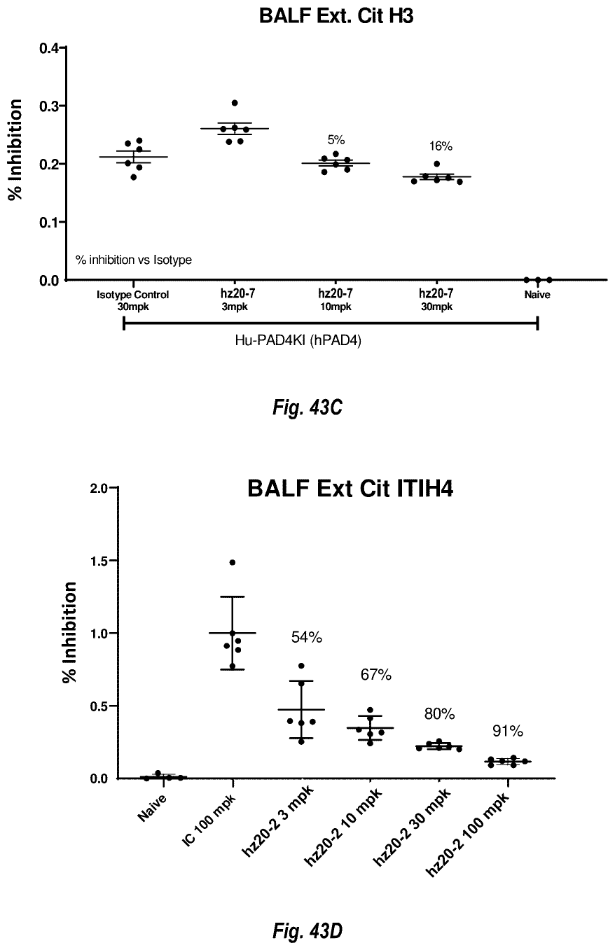

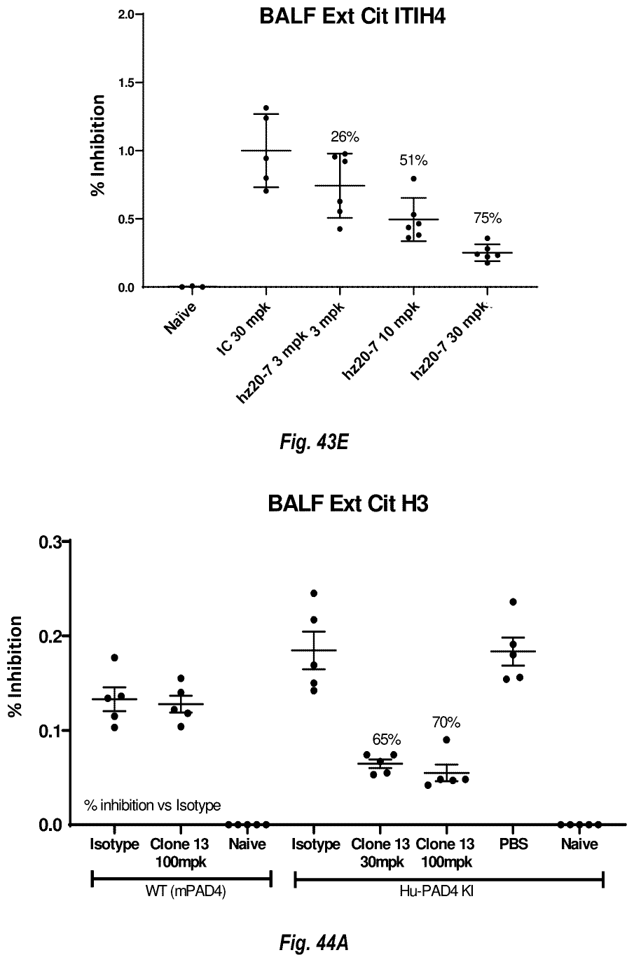

Specifically, mAb20 reduced extracellular Cit H3 by 41% in Hu-PAD4 KI mice at the lowest dose tested (30 mg/kg) (FIG. 43A). This activity was specific to human PAD4 since even at 100 mg/kg, mAb20 did not reduce extracellular Cit-H3 in WT mice. The humanized derivatives of mAb20, hz20-2 (FIG. 43B) and hz20-7 (FIG. 43C), showed 12% and 16% reduction of extracellular Cit-H3, respectively, at 30 mg/kg, and hz20-2 showed a 48% reduction of extracellular Cit-H3 at 100 mg/kg (FIG. 43B). Antibodies hz20-2 and hz20-7 also reduced extracellular Cit-ITIH4. At 30 mg/kg, hz20-2 (FIG. 43D) and hz20-7 (FIG. 43E) reduced extracellular Cit-ITIH4 by 80% and 75%, respectively.

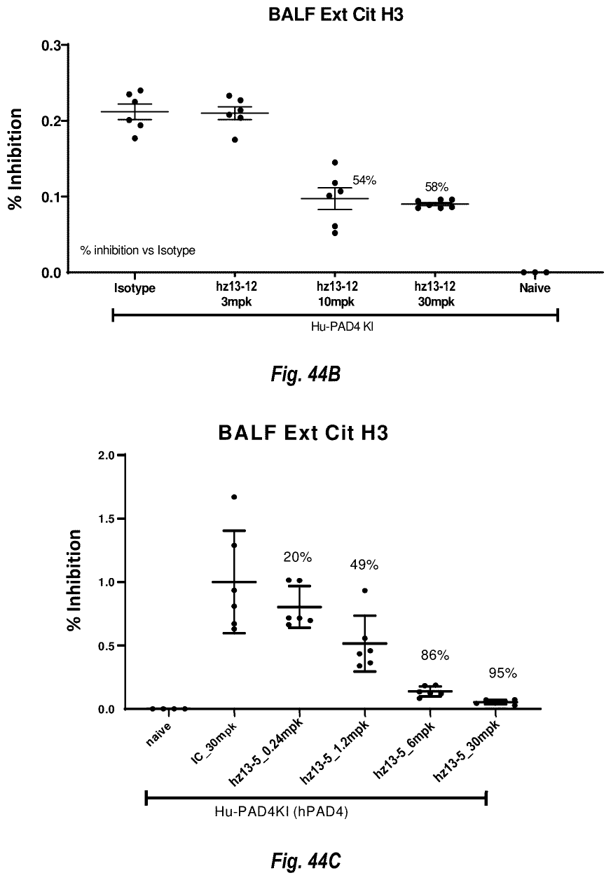

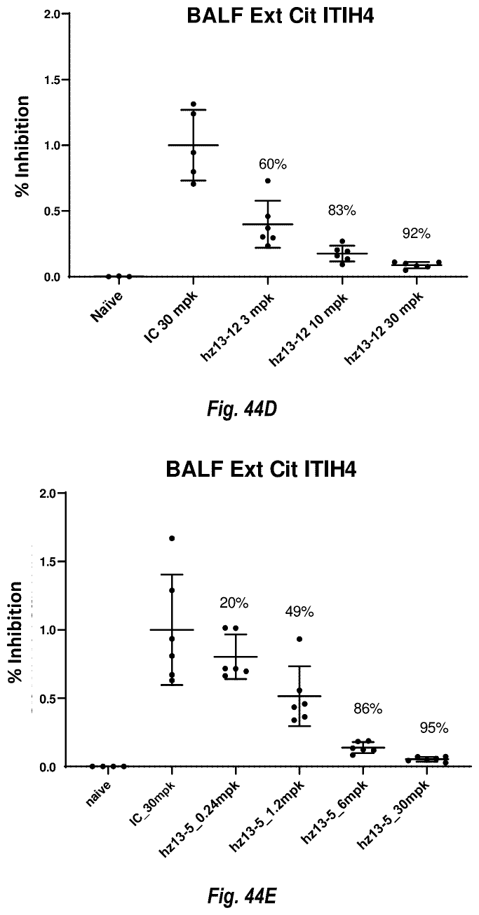

In addition, mAb 13 reduced extracellular Cit-H3 in Hu-PAD4 KI mice but not in WT mice (FIG. 44A). mAb 13 reduced extracellular Cit-H3 by 65% and 70% at 30 mg/kg and 100 mg/kg, respectively (FIG. 44A). A first humanized derivative of mAb13, hz13-12, was similarly effective in reducing extracellular Cit-H3, demonstrating a 54% reduction at 10 mg/kg and a 58% reduction at 30 mg/kg (FIG. 44B). A second humanized derivative of mAb13, hz13-5, had an even greater impact on extracellular Cit-H3 reduction by demonstrating 86% reduction at 6 mg/kg and 95% at 30 mg/kg (FIG. 44C). Similarly, both hz13-12 and hz13-5 reduced extracellular Cit-ITIH4 by more than 80% at a dose of 6 mg/kg dose and above (FIG. 44D-FIG. 44E).

The results show that anti human PAD4 mAbs modulated PAD4 function in a preclinical LPS ALI model of inflammation. Specifically, the antibodies reduced the level of extracellular Cit-H3.

Table 17 summarizes % reduction of extracellular Cit-H3 and Cit-ITIH4, as shown in FIG. 43A-FIG. 43E and FIG. 44A-FIG. 44E.

TABLE 17
In Vivo Activity of Clone 13 and 20 parent and derivative
Abs in LPS ALI PD model using the Hu-PAD4KI mice.

| | | Cit-H3 | ITIH4 |
|---|---|---|---|
| Antibody | Concentration | % Reduction | % Reduction |
| mAb20 | 30 | 41 | ND |
| hz20-7 | 30 | 16 | 75 |
| hz20-2 | 30 | 12 | 80 |
| mAb13 | 30 | 65 | ND |
| hz13-12 | 10 | 54 | 83 |
| hz13-5 | 6 | 40 | 86 |

Data shows concentration (or maximal dose tested) at which~50% inhibition was observed for Cit-H3 and Cit-ITIH4 for Clone 13 and 20 parent and derivatives tested at the LPS ALI PD model.
“ND” means not determined.

### Example 26: Activity of Anti-Human PAD4 Antibodies in an LPS-Induced Acute Joint Inflammation Model

The LPS-induced AJI model was used to determine in vivo activity of anti-human PAD4 antibodies (Ab) in the joints of human PAD4 knock-in (HU-PAD4KI) mice.

A. Methods Related to the LPS AJI Model

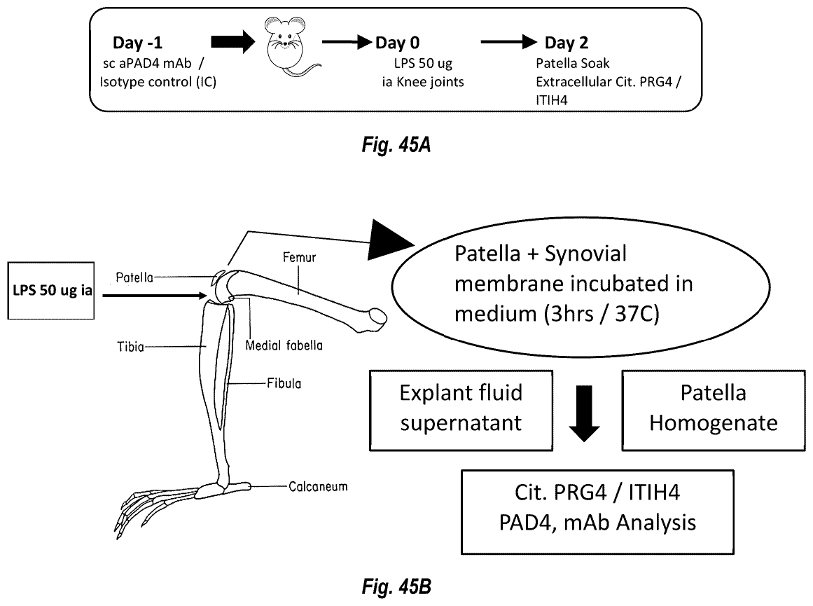

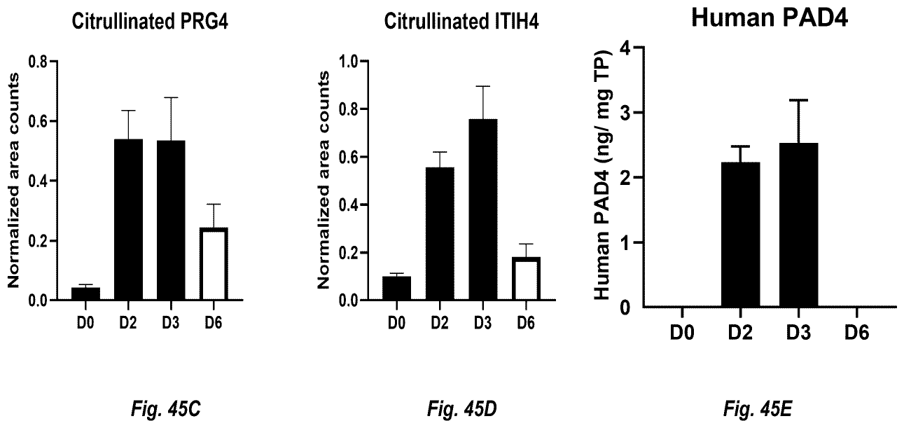

The methods related to the LPS-induced AJI model in this Example (FIG. 45A-FIG. 45B) were as described in Example 21 above with the following modifications: C57Bl/6 human-PAD4 knock-in (Hu-PAD4 KI) mice were used as well as WT mice. At multiple time points post is injection, mice patella were removed and explanted to extract extra-cellular proteins (FIG. 45B), and Cit-PRG4, Cit-ITIH4, and hPAD4 expression was determined by LC/MS (FIG. 45C-FIG. 45E).

In a first experiment, the following antibodies were tested: the humanized derivatives of clone 13 hz13-5 and hz13-12, and the humanized derivative of clone 20 hz20-2. Mice were treated with these Abs subcutaneously at 1 mg/kg, 3 mg/kg, 10 mg/kg, 30 mg/kg, or 100 mg/kg. Control mice were treated with an isotype control murine antibody (IC mAb) at 100 mg/kg. In a second experiment, hz13-5 D31E was tested and hz13-5 was tested at the lowest dose administered. Mice were treated with hz13-5 D31E subcutaneously at 0.1 mg/kg, 1 mg/kg, or 30 mg/kg, or with hz13-5 at 0.1 mg/kg.

B. Results

First, mice were treated with LPS without antibodies to determine the ideal time frame for measuring expression levels of citrullinated proteins. Peak expression levels of Cit-PRG4, Cit-ITIH4, and hPAD4 (FIG. 45C-FIG. 45E) were observed at days 2-3 after ia injection of LPS. The expression levels declined by day 6 post ia injection. Hence, day 2 was determined as ideal time to determine Cit-PRG4 and Cit-ITIH4 protein expression.

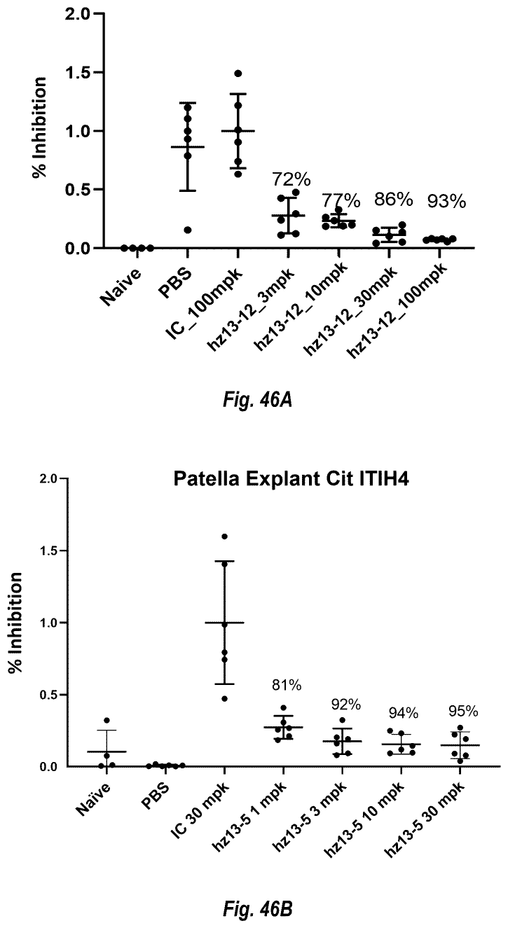

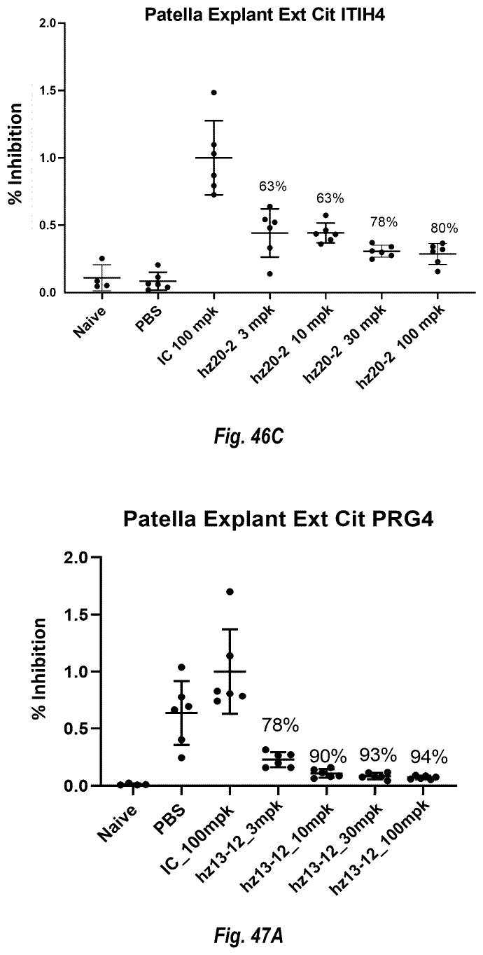

Next, in order to test the ability of the antibodies to inhibit PAD4 function in the LPS AJI PD model, the antibodies were administered as described in the method section above and as shown in FIG. 45A-FIG. 45B. Clone 13-based hz13-12 antibody (FIG. 46A), clone 13-based hz13-5 antibody (FIG. 46B) and clone 20-based hz20-2 antibody (FIG. 46C) reduced relative levels of Cit-ITIH4 in a dose-dependent manner. Clone 13-based hz13-5 had the strongest effects, providing 81% reduction at 1 mg/kg and 95% reduction at 30 mg/kg (FIG. 46B).

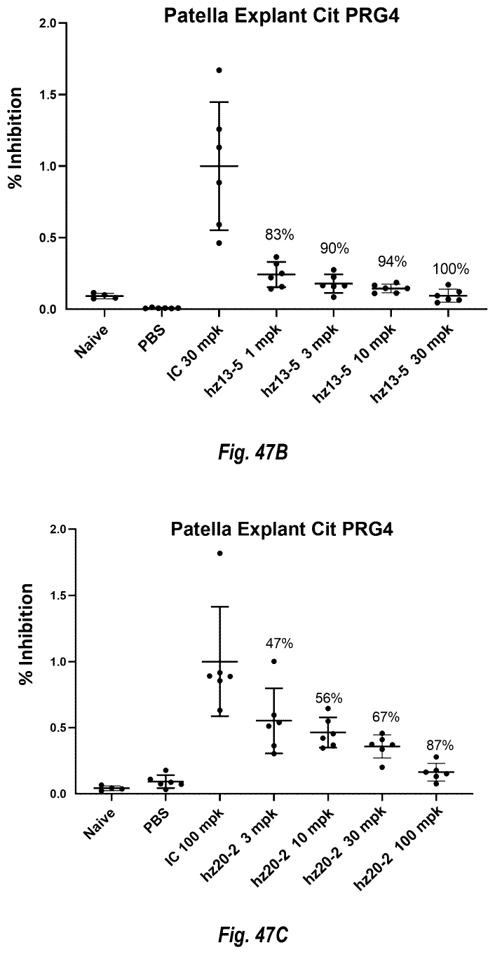

In the first experiment, clone 13-based hz13-12 antibody (FIG. 47A), clone 13-based hz13-5 antibody (FIG. 47B), and clone 20-based hz20-2 antibody (FIG. 47C) also reduced relative levels of Cit-PRG4 in a dose-dependent manner. Clone 13-based hz13-5 had the strongest effects, providing 83% reduction at 1 mg/kg and 100% reduction at 30 mg/kg (FIG. 47B).

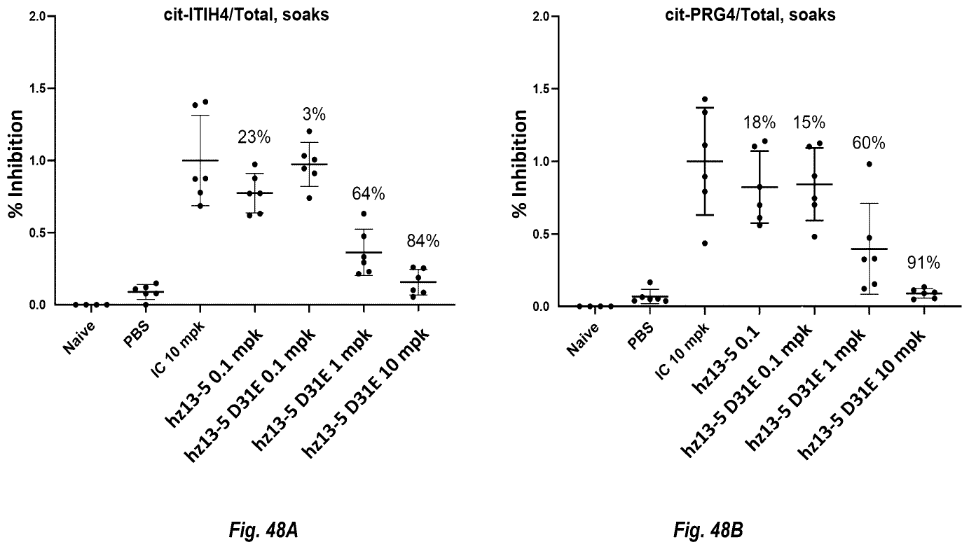

In the second experiment, both hz13-5 D31E and hz13-5 inhibited citrullination of ITIH4 (FIG. 48A) and PRG4 (FIG. 48B). The antibody hz13-5 D31E showed dose dependent inhibition of citrullination of ITIH4 and PRG4.

The results as a whole indicate that anti human PAD4 mAbs inhibited PAD4 function in this LPS-induced AJI in vivo model.

### Example 27: Therapeutic PAD4 Antibody Maintained Potency in Presence of Endogenous PAD4 Antibodies from Rheumatoid Arthritis Patients

A. Introduction

PAD4 is released extracellularly in the inflamed joint through neutrophil activation, NETosis and cell death. PAD enzymes catalyze the modification of an arginine into a citrulline residue and drive the formation of citrullinated neoantigens that are recognized by anti-citrullinated peptide antibodies (ACPA), which are a hallmark of rheumatoid arthritis. Formation of immune complexes between ACPAs and citrullinated proteins are believed to drive tissue damage and perpetuate inflammation. Besides ACPA, about 25-35% of RA patients express anti-PAD4 IgG, of which about 20-40% cross-react between PAD3/PAD4 and have the potential to enhance PAD4 activity leading to more erosive disease. In this Example, the impact of the presence of endogenous anti-PAD4 antibodies on the potency of antibody hz13-5 D31E was investigated in serum or with purified immunoglobin G (IgG).

B. Materials and Methods

a. Serum Samples

The samples were serum samples from rheumatoid arthritis (RA) patients with established disease. Serum from healthy controls (NHV) was collected in BD Vacutainer SST Blood collection tubes (Cat #367988). All serum samples were frozen until aliquoting in plates.

b. PAD4 Antibody ELISA

Serum anti-PAD4 autoantibodies were measured using PAD4 Autoantibody ELISA kit (500930) from Cayman Chemicals following manufacturer's protocol. Serum samples were tested at 1:150 dilution in an assay buffer provided in the ELISA kit.

c. IgG purification

Human IgG was purified using Melon™ gel IgG Spin Purification Kit (45206) from Thermo Scientific. Briefly, 500 μl purification gel was loaded to spin columns and washed twice with 300 μl of purification buffer. 80 μl of serum was diluted 5-fold with purification buffer and added to purification gel containing spin columns. Columns were mixed end-over-end for 5 minutes and centrifuged to collect purified IgG. Concentration of purified IgG was determined using NanoDrop™ One instrument from Thermo Scientific.

d. Citrullinated H3 Enzymatic Assay

All reagents were calculated for 100 μl final reaction. In 42.5 μl volume, recombinant PAD4 (Cayman #10500) was pre-incubated with purified IgG on ice for 45 minutes. To produce inhibition curves, 7.5 μl of hz13-5 D31E serial dilutions in PBS or isotype control were added to the PAD4/IgG mix and incubated on ice for an additional 45 minutes. Histone H3 in buffer containing calcium chloride was added, and the reaction (final concentrations: 13.5 nM PAD4, 2 mM CaCl2, 10 μg/ml H3, 100 μg/ml purified IgG) was incubated at 37° C. in an incubator for 2 hours. The H3 citrullination reaction was stopped with 10 μl of 0.5M EDTA. Citrullinated histone H3 was measured using Citrullinated Histone H3 (Clone 11D3) ELISA kit (501620) from Cayman Chemicals following manufacturer's protocol. Citrullinated samples were tested @ 1:100 dilution in an assay buffer provided in the ELISA kit.

e. Citrullination Induction in Serum

To produce inhibition curves, 7.5 μl of hz13-5 D31E serial dilutions in PBS or isotype control were added to 80 μl of thawed serum in plates, mixed, incubated at 37° C. in CO₂ incubator for 6 h then stored overnight at −80° C. Plates containing serum mixed with antibody were thawed and 12.2 μl of PAD4 enzyme reaction mix (13.5 nM PAD4, 100 mM Tris, 2 mM DTT final) was added and incubated at 37° C. for 2 h. Reactions were stopped with 10 μl of 0.5 M EDTA. Plates were sealed and stored at −80° C.

f. Measure of Citrullinated Peptides by LC-MS

Ten (10) μl of serum was thawed and diluted 15-fold with PBS. Sixty (60) μl of 15-fold diluted plasma was used for depletion of human serum albumin using CUSTOM PhyTip 200 μL CaptureSelect™ Human Albumin tips (Biotage, San Jose, CA) containing 20 CaptureSelect™ human albumin affinity matrix. Five (5) μl of albumin-depleted serum was combined with 45 ul LYSE BCT buffer and heated for 20 minutes at 80° C. Samples were enzymatically digested with addition of trypsin and LysC according to manufacturer's protocol. Peptides were cleaned up using preOmics BCT kit (cat #P.0.00116).

Peptides were analyzed by nanoLC-MS on Bruker's timsTOF of mass spectrometer using a 30-minute prm-PASEF method. Chromatographic separation was done using Bruker's NanoElute™ LC on AURORA ELITE (AUR-15075C18-CSI) column. Data dependent acquisition (DDA) data was acquired for samples with high PAD4 concentrations with spiked internal standards (heavy peptides) for each peptide of interest. Skyline® software was used to generate a list of cit-peptide (citrullinated peptide) and unmodified peptide sequences from cit-proteins previously identified by global proteomics experiments. Next, the library in Skyline® was created using MaxQuant® generated msms.txt; mqpar.xml, and modifications.xml files. Ion mobility library was created after importing DDA data and using the imported data to generate a spectra library. Three parameters for each analyte were exported from Skyline® to build a prm-PASEF method in TimsTOF Control 3.0 software: retention time (RT); Ion Mobility (IM), and precursor m/z. Data was analyzed using Skyline® workflow.

g. Calculation of IC50

IC50 calculations and statistics were performed in GraphPad® Prism software. Citrullinated H3 or % citrullination were plotted against the log of the concentration of the antibody and a non linear fit (Hill Slope=−1) was performed.

C. Results

a. PAD4 Antibody Status

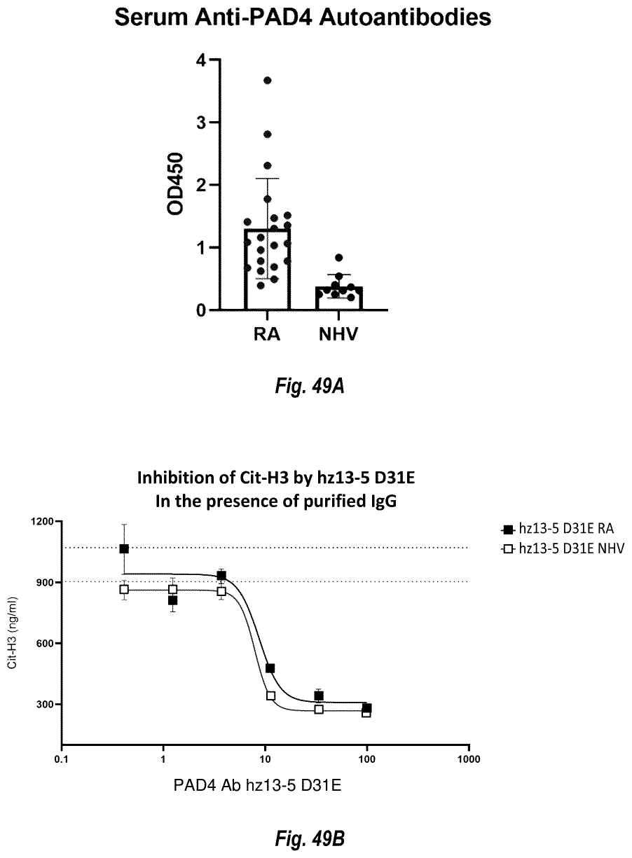

The presence of endogenous PAD4 autoantibodies was evaluated in the serum samples from the 21 RA patients and 10 healthy controls (NHV) by ELISA. On average the OD was significantly higher in RA than in NHV (1.3 in RA vs 0.38 in NHV, p<0.0001, Mann Whitney T test). Using 1 as a cutoff for positivity, 13 out of 21 (62%) RA sera and none of the NHV sera could be considered anti-PAD4 IgG+. FIG. 49A shows serum anti-PAD4 autoantibodies measured by OD450 by ELISA in 21 RA and 10 NHV purified IgG.

b. Hz13-5 D31E Potency in the Presence of Purified IgG

Potency of hz13-5 D31E on inhibition of H3 citrullination in the presence of 100 ug/ml purified IgG from the 21 RA and 6 NHV sera was evaluated (3 plates containing each 7 RA and 2 NHV donors). hz13-5 D31E inhibited in vitro PAD4 driven H3 citrullination in the presence of purified IgG and there was no difference in IC50 whether purified IgG came from RA or NHV sera (FIG. 49B, Table 18), despite the fact that a large proportion of RA IgG had anti-PAD4 autoantibodies. The mean IC50 (nM) for the RA sera (n=21) was 8.2 (standard deviation of 1.3), while the mean IC50 (nM) for the NHV sera (n=6) was 9.0 (with standard deviation 2.5).

c. Hz13-5 D31E Potency in Serum from RA Patients

Serum from 21 RA and 10 NHV donors was incubated with 13.5 nM PAD4 to induce citrullination, without or with different doses of hz13-5 D31E. Citrullination of 5 selected peptides for proteoglycan 4 (PRG4), fibrinogen A (FGA), Inter-alpha-trypsin inhibitor heavy chain H4 (ITIH4), Alpha-1-microglobulin/bikunin precursor (AMBP) and gelsolin (GSN) was measured by LC-MS and reported as a percent of citrullinated over total peptide. Citrullination of these peptides was observed in all samples with inter-donor variability. hz13-5 D31E inhibited citrullination of each of these peptides in a dose dependent manner and potency similar between NHV and RA (Table 18, no statistical differences between NHV and RA by Mann Whitney test). In addition, there was no correlation between any of the IC50s and the presence of anti-PAD4 autoantibodies (Spearman, not shown), as measured by ELISA (FIG. 49A), suggesting that the presence of endogenous PAD4 antibodies do not affect potency of hz13-5 D31E. Table 18 shows IC50 of hz13-5 D31E on citrullination of 5 serum peptides from RA or NHV donors. SD refers to the standard deviation. The sequences shown in Table 18 are SEQ ID NOs: 236-240, respectively, from top to bottom.

TABLE 18

| | Peptide sequence | IC50 (nM) | |
|---|---|---|---|
| | (citrullination | mean (SD) | IC50 (nM) |
| | sites that were | for | mean (SD) |
| Pro- | assessed are | RA | for NHV |
| tein | underlined) | (n = 21) | (n = 10) |
| PRG4 | AITTRSGQTLSK | 4.9 (3.5) | 4.4 (2.3) |
| FGA | QFTSSTSYNRGDSTFESK | 4.5(1.6) | 5.5(2.3) |
| ITIH4 | QLGLPGPPDVPDHAAYHPFRR | 5.2(1.5) | 5.7(2.6) |
| AMBP | GPCRAFIQLWAFDAVK | 4.3(1.0) | 5.1(2.1) |
| GSN | ATASRGASQAGAPQGR | 7.1(3.6) | 7.7(9.3) |

With two independent assays, no shift in potency of hz13-5 D31E in the presence of endogenous anti-PAD4 autoantibodies was observed in this set of 21 RA serum samples.

### Example 28: Cross-Reactivity Study of Anti-PAD4 Antibody Hz13-5 D31E with Various Human Tissues

Potential cross-reactivity of hz13-5 D31E to a panel of normal human tissues was assessed using a fluoresceinated hz13-5 D31E (hz13-5 D31E FITC). Hz13-5 D31E FITC was applied to cryosections of normal human tissues from at least 3 donors per tissue (where available) at 1 μg/mL and at 5 μg/mL At both concentrations, hz13-5 D31E FITC moderately to intensely stained the cytoplasm and nucleus of frequent mononuclear and polymorphonuclear leukocytes in human peripheral blood. The experiments showed that hz13-5 D31E FITC stained the cytoplasm and nucleus of polymorphonuclear leukocytes in most of the human tissue samples, stained the cytoplasm of rare mononuclear leukocytes in the lymph node (mainly sinus macrophages) and in peripheral blood, and stained the cytoplasm of reticulo-endothelial cells in the spleen. These results are generally consistent with the reported expression of PAD4 in the cytoplasm and nucleus of granulocytes and in monocytes in peripheral blood. (Jones et al. Curr. Opin. Drug Discov. Devel. 12(5): 616-27 (2009); Vossenaar et al., Ann. Rheum. Dis. 63(4): 373-81; Zhou et al., Front. Immunol. 8: 1200 (2017).) Moreover, no membrane binding with hz13-5 D31E FITC was observed in any of the human tissues examined. These results indicate that hz13-5 D31E FITC does not cross-react with normal human tissues.

### Example 29: Assessment of Hz13-5 D31E Antibody in In Vitro Respiratory Burst and Phagocytosis in Human Whole Blood

The potential of hz13-5 D31E to impact respiratory burst and phagocytosis in an in vitro human whole blood assay was assessed.

Phagocytosis was not induced by neutrophils incubated with hz13-5 D31E or an isotype control antibody (human IgG1.3f antibody), followed by incubation with opsonized conjugated E. coli bioparticles on ice. There were also no differences in phagocytosis from whole blood samples incubated with hz13-5 D31E, isotype control, or media control followed by incubation with E. coli bioparticles at 37° C.

A respiratory burst positive control, PMA, induced respiratory burst in neutrophils in whole blood samples from all donors, which was inhibited by the respiratory burst inhibitor Kaempferol. Respiratory burst was not induced by PMA in the presence of hz13-5 D31E or isotype control antibody at the tested concentrations of 0.128 to 400 μg/mL. And hz13-5 D31E and isotype control antibody alone did not induce respiratory burst at a concentration of 400 μg/mL, which was the highest concentration tested, and which is also more than twice the predicted Cmax of hz13-5 D31E in a 900 mg human dose.

These results indicate that hz13-5 D31E has no impact on innate immune functions of phagocytosis and respiratory burst at the concentrations tested in this model.

### Example 30: PAD4 Antibody in Combination with Checkpoint Inhibitors Inhibited Citrullination in an In Vivo Breast Cancer Model

The 4T1 breast cancer syngeneic model was used to investigate anti-tumor activity of checkpoint inhibitors with and without inhibition of PAD4 using an anti-mouse PAD4 monoclonal antibody (the mumAb described herein) or small molecule PAD4 inhibitor. The 4T1 model is described in Teijeira et al. (2020) Immunity: 56, 856-871. When introduced orthotopically in the mammary fat pad the tumor grows both at the primary site and is also known to metastasize to liver, lungs and lymph nodes.

4T1 cells were maintained in in RPMI-1640 medium with 10% FBS. Balb/c mice 6-8 weeks old were injected with 5×10⁴ cells in 50 μL serum and phenol red free media subcutaneously into the mammary fat pad. Tumor measurements were taken 3 times per week starting at day 7 post-implant, and animals are randomized into treatment groups at 100 mm 3 mean tumor volume (expected tumor range ˜70-150 mm 3).

The treatment protocol is provided in Table 19 below. The treatments used in this study include anti-mPD-1 antibody (aPD-1) that is a murinized equivalent to nivolumab, anti-mCTLA-4 antibody (aCTLA-4) that is a murinized equivalent to ipilimumab, a small molecule PAD4 inhibitor GSK484, and an antibody inhibitor of PAD4 (mumAb), as well as controls (GSK484 diluent, mumAb isotype control). Mice of Group I in Table 19 received isotope control antibodies against the KLH antigen (keyhole limpet hemocyanin). Groups 2-7 of Table 19 received the listed treatments in the Table, corresponding to various anti-tumor treatment combinations. The treatments listed in the second column were administered, respectively, at the doses listed in the fourth column and the frequencies listed in the fifth column. Groups 1-3 and 6-7 were designed to have n=5 tumors harvested at an earlier timepoint (day 19 post implantation) for PD assessment. The rest of the n=15 mice in all groups were monitored for tumor growth.

TABLE 19
Treatment Protocol

| | | | Dose | | |
|---|---|---|---|---|---|
| | | | mg/kg/day | | |
| | | | of each | | |
| | | # | respective | Regimen for each | |
| Group | Treatment | Mice | treatment | respective treatment | Route |
| 1 | Control KLH mIgG1 D265A + | 20 | 10 + 10 | QWx3 + QWx3 + QDx7 | IP |
| | KLH mIgG2b + GSK diluent | | | | |
| 2 | aPD-1 + aCTLA-4 + | 20 | 10 + 10 | QWx3 + QWx3 + QDx7 | IP |
| | GSK diluent | | | | |
| 3 | aPD-1 + aCTLA-4 + | 20 | 10 + 10 + 5 | QWx3 + QWx3 + QDx7 | IP |
| | GSK484 | | | | |
| 4 | aPD-1 + aCTLA-4 + isotype | 15 | 10 + 10 + 100 | QWx3 + QWx3 + QWx3 | IP |
| | control | | | | |
| 5 | aPD-1 + aCTLA-4 + mumAb | 15 | 10 + 10 + 30 | QWx3 + QWx3 + QWx3 | IP |
| 6 | aPD-1 + aCTLA-4 + mumAb | 20 | 10 + 10 + 100 | QWx3 + QWx3 + QWx3 | IP |
| 7 | aPD-1 + aCTLA-4 + mumAb | 20 | 10 + 10 + 100 | QWx3 + QWx3 + QWx3 | IP/SC¹ |

¹aPD-1 and aCTLA-4 administered IP, mumAb administered SC;
KLH = keyhole limpet hemocyanin

Differences between treatment groups were assessed using outcome measures (i) tumor size, and (ii) immunohistochemistry of citrullinated DNA from resected tumors. Compared with checkpoint inhibitor treatment alone, combinations including PAD4 inhibitor did not show improved tumor growth inhibition. Of note, anti-drug antibody (ADA) formation contributed to fatal anaphylaxis following the 3^{rd} dose (day 24 post implantation) of the combination of aPD-1 and aCTLA4 (this combination is referred to in this Example as the checkpoint inhibitor doublet or CPI doublet). Mice were lost in each group treated with the CPI doublet, indicating that the ADA was not against the mumAb. Peripheral mouse exposure levels up until the 3^{rd} dose of CPI doublet showed values above the predicted efficacious range. The groups that received the PAD4 inhibiting agents mumAb (groups 5, 6, and 7) or GSK484 (group 3) did not show enhanced tumor growth inhibition compared with their corresponding control groups (groups 4 and 2, respectively).

In the groups harvested on day 19 post-implantation a trend was observed in tumor histone H3 citrullination, with a significant decrease in Group 7 (the combination of high-dose aPAD4 mumAb at 100 mg/kg combined with CPI doublet) compared with CPI doublet and isotype control (Group 4). This result indicates that administering PAD4 antibody at a sufficient dose can inhibit citrullination in tumors.

### REFERENCES

R. Huang and G. Chen, Higher order structure characterization of protein therapeutics by hydrogen/deuterium exchange mass spectrometry. Analytical and Bioanalytical Chemistry, 2014, 406, 6541-6558.
H. Wei, J. Mo, L. Tao, R. J. Russell, A. A. Tymiak, G. Chen, R. E. Iacob, J. R. Engen, Hydrogen/deuterium exchange mass spectrometry for probing higher order structure of protein therapeutics: methodology and applications. Drug Discovery Today, 2014, 19, 95-102.
L. M. Jones, J. B. Sperry, J. A. Carroll, M. L. Gross, Fast photochemical oxidation of proteins for epitope mapping. Analytical Chemistry, 2011, 83, 7657-7661.
H. Zhang, J. Wen, R. Huang, R. Blankenship, M. Gross, Mass spectrometry-based carboxyl footprinting of proteins: Method evaluation. International Journal of Mass Spectrometry, 2012, 312, 78-86.
C. Y. Tremblay, P. Limpikirati, R. W. Vachet. J. Am. Soc. Mass Spectrom. 2021, 32, 5, 1237-1248
Xu L et al. (2002) Chemistry & Biology 9: 933
Roberts R W and J W Szostak (1997) Proc. Natl. Acad. Sci. USA 94:12297
Kurz et al. (2000) Nucleic Acids Res. 28(18):E83
Joubert, M. K., et al., Use of In Vitro Assays to Assess Immunogenicity Risk of Antibody-Based Biotherapeutics. PLoS One, 2016. 11(8): p. e0159328.
Wang, P., et al., A systematic assessment of WIC class II peptide binding predictions and evaluation of a consensus approach. PLoS Comput Biol, 2008. 4(4): p. e1000048.
De Groot, A. S. and W. Martin, Reducing risk, improving outcomes: bioengineering less immunogenic protein therapeutics. Clin Immunol, 2009. 131(2): p. 189-201.
Hua, F., et al., Anti-IL21 receptor monoclonal antibody (ATR-107): Safety, pharmacokinetics, and pharmacodynamic evaluation in healthy volunteers: a phase I, first-in-human study. J Clin Pharmacol, 2014. 54(1): p. 14-22.
Prometheus, July 2012, Proleukin® (aldesleukin), accessdatafda.gov/drugsatfda docs/label/2012/103293s5130lbl.pdf.
Li, M., Lin, C., Deng, H., Strnad, J., Bernabei, L., Vogl, D. T., Burke, J. J., & Nefedova, Y. (2020). A Novel Peptidylarginine Deiminase 4 (PAD4) Inhibitor BMS-P5 Blocks Formation of Neutrophil Extracellular Traps and Delays Progression of Multiple Myeloma. Molecular cancer therapeutics, 19(7), 1530-1538. https://doi.org/10.1158/1535-7163.MCT-19-1020
Teijeira, Á., Garasa, S., Gato, M., Alfaro, C., Migueliz, I., Cirella, A., de Andrea, C., Ochoa, M. C., Otano, I., Etxeberria, I., Andueza, M. P., Nieto, C. P., Resano, L., Azpilikueta, A., Allegretti, M., de Pizzol, M., Ponz-Sarvise, M., Rouzaut, A., Sanmamed, M. F., Schalper, K., . . . Melero, I. (2020). CXCR1 and CXCR2 Chemokine Receptor Agonists Produced by Tumors Induce Neutrophil Extracellular Traps that Interfere with Immune Cytotoxicity. Immunity, 52(5), 856-871.e8. https://doi.org/10.1016/j.immuni.2020.03.001
Wang, Y., Liu, C., Zhang, N., Song, X., Song, Y., Cai, D., Fang, K., & Chang, X. (2022). Anti-PADI4 antibody suppresses breast cancer by repressing the citrullinated fibronectin in the tumor microenvironment. Biomedicine & pharmacotherapy=Biomedecine & pharmacotherapie, 153, 113289. Advance online publication. https://doi.org/10.1016/j.biopha.2022.113289
Yazdani, H. O., Roy, E., Comerci, A. J., van der Windt, D. J., Zhang, H., Huang, H., Loughran, P., Shiva, S., Geller, D. A., Bartlett, D. L., Tsung, A., Sheng, T., Simmons, R. L., & Tohme, S. (2019). Neutrophil Extracellular Traps Drive Mitochondrial Homeostasis in Tumors to Augment Growth. Cancer research, 79(21), 5626-5639. https://doi.org/10.1158/0008-5472.CAN-19-0800

### DESCRIPTION OF THE SEQUENCES

The following table provides a listing of certain sequences referenced herein. In the antibody variable region sequences disclosed herein, the heavy chain variable region (VH) CDR1, CDR2, and CDR3 sequences are located at Kabat positions comprising amino acids 26-35, 50-65, and 95-102, respectively, as shown in FIG. 1E, and which correspond to amino acid positions 26-35, 50-66, and 99-108 of SEQ ID NO: 10 and NO: 78 in the table below, and the light chain variable region (VL) CDR1, CDR2, and CDR3 sequences are located at Kabat positions comprising amino acids 24-34, 50-56, and 89-97, respectively, as shown in FIG. 1F, and which correspond to amino acid positions 24-38, 54-60, and 93-101 of SEQ ID NO: 12 and NO: 80 in the table below. In certain VH and VL sequences, the CDR sequences are in bold text, in some cases also with underlining. In certain humanized antibody VH and VL sequences, underlined amino acids in the framework regions indicate framework back mutations to mouse amino acids. In certain full-length heavy and light chain sequences, variable regions are in italics. In certain mutant constant region sequences, constant region residues that are altered compared to the corresponding wild-type sequence are shown in bold underlining.

Sequence Table.

| ID | | |
|---|---|---|
| NO. | Description | Sequence |
| 1 | Human protein arginine | MAQGTLIRVTPEQPTHAVCVLGTLTQLDICSSAPEDCTSFSINASPGVV |
| | deiminase type 4 | VDIAHGPPAKKKSTGSSTWPLDPGVEVTLTMKVASGSTGDQKVQISYYG |
| | (huPAD4; UniProt ID: | PKTPPVKALLYLTGVEISLCADITRTGKVKPTRAVKDQRTWTWGPCGQG |
| | Q9UM07) | AILLVNCDRDNLESSAMDCEDDEVLDSEDLQDMSLMTLSTKTPKDFFTN |
| | | HTLVLHVARSEMDKVRVFQATRGKLSSKCSVVLGPKWPSHYLMVPGGKH |
| | | NMDFYVEALAFPDTDFPGLITLTISLLDTSNLELPEAVVFQDSVVFRVA |
| | | PWIMTPNTQPPQEVYACSIFENEDFLKSVTTLAMKAKCKLTICPEEENM |
| | | DDQWMQDEMEIGYIQAPHKTLPVVFDSPRNRGLKEFPIKRVMGPDFGYV |
| | | TRGPQTGGISGLDSFGNLEVSPPVTVRGKEYPLGRILFGDSCYPSNDSR |
| | | QMHQALQDFLSAQQVQAPVKLYSDWLSVGHVDEFLSFVPAPDRKGFRLL |
| | | LASPRSCYKLFQEQQNEGHGEALLFEGIKKKKQQKIKNILSNKTLREHN |
| | | SFVERCIDWNRELLKRELGLAESDIIDIPQLFKLKEFSKAEAFFPNMVN |
| | | MLVLGKHLGIPKPFGPVINGRCCLEEKVCSLLEPLGLQCTFINDFFTYH |
| | | IRHGEVHCGTNVRRKPFSFKWWNMVP |
| 2 | Full-length PAD4 | MAQGTLIRVTPEQPTHAVCVLGTLTQLDICSSAPEDCTSFSINASPGVV |
| | construct | VDIAHSPPAKKKSTGSSTWPLDPGVEVTLTMKAASGSTGDQKVQISYYG |
| | | PKTPPVKALLYLTAVEISLCADITRTGKVKPTRAVKDQRTWTWGPCGQG |
| | | AILLVNCDRDNLESSAMDCEDDEVLDSEDLQDMSLMTLSTKTPKDFFTN |
| | | HTLVLHVARSEMDKVRVFQATRGKLSSKCSVVLGPKWPSHYLMVPGGKH |
| | | NMDFYVEALAFPDTDFPGLITLTISLLDTSNLELPEAVVFQDSVVFRVA |
| | | PWIMTPNTQPPQEVYACSIFENEDFLKSVTTLAMKAKCKLTICPEEENM |
| | | DDQWMQDEMEIGYIQAPHKTLPVVFDSPRNRGLKEFPIKRVMGPDFGYV |
| | | TRGPQTGGISGLDSFGNLEVSPPVTVRGKEYPLGRILFGDSCYPSNDSR |
| | | QMHQALQDFLSAQQVQAPVKLYSDWLSVGHVDEFLSFVPAPDRKGFRLL |
| | | LASPRSCYKLFQEQQNEGHGEALLFEGIKKKKQQKIKNILSNKTLREHN |
| | | SFVERCIDWNRELLKRELGLAESDIIDIPQLFKLKEFSKAEAFFPNMVN |
| | | MLVLGKHLGIPKPFGPVINGRCCLEEKVCSLLEPLGLQCTFINDFFTYH |
| | | IRHGEVHCGTNVRRKPFSFKWWNMVP |
| 3 | Full-length PAD4 | MGSSHHHHHHSSGETVRFQGHMAQGTLIRVTPEQPTHAVCVLGTLTQLD |
| | construct with cleavable | ICSSAPEDCTSFSINASPGVVVDIAHSPPAKKKSTGSSTWPLDPGVEVT |
| | N-terminal his tag and | LTMKAASGSTGDQKVQISYYGPKTPPVKALLYLTAVEISLCADITRTGK |
| | TMVM tag | VKPTRAVKDQRTWTWGPCGQGAILLVNCDRDNLESSAMDCEDDEVLDSE |
| | | DLQDMSLMTLSTKTPKDFFTNHTLVLHVARSEMDKVRVFQATRGKLSSK |
| | | CSVVLGPKWPSHYLMVPGGKHNMDFYVEALAFPDTDFPGLITLTISLLD |
| | | TSNLELPEAVVFQDSVVFRVAPWIMTPNTQPPQEVYACSIFENEDFLKS |
| | | VTTLAMKAKCKLTICPEEENMDDQWMQDEMEIGYIQAPHKTLPVVFDSP |
| | | RNRGLKEFPIKRVMGPDFGYVTRGPQTGGISGLDSFGNLEVSPPVTVRG |
| | | KEYPLGRILFGDSCYPSNDSRQMHQALQDFLSAQQVQAPVKLYSDWLSV |
| | | GHVDEFLSFVPAPDRKGFRLLLASPRSCYKLFQEQQNEGHGEALLFEGI |
| | | KKKKQQKIKNILSNKTLREHNSFVERCIDWNRELLKRELGLAESDIIDI |
| | | PQLFKLKEFSKAEAFFPNMVNMLVLGKHLGIPKPFGPVINGRCCLEEKV |
| | | CSLLEPLGLQCTFINDFFTYHIRHGEVHCGTNVRRKPFSFKWWNMVP |
| 4 | HCDR1 mAb13 | DFNIKDHFIH |
| 5 | HCDR2 mAb13 | RIDPEDDETKYAPKFQD |
| 6 | HCDR3 mAb13 | YGNYEGAMDY |
| 7 | LCDR1 mAb13 | RASENVNNYGIGFMN |
| 8 | LCDR2 mAb13 | AASRRGS |
| 9 | LCDR3 mAb13 | QQSKEVPYT |
| 10 | mAb13 heavy chain | EVLLQQSGAELVKPGASVRLSCTSSDFNIKDHFIHWVKQRTEQGLEWIG |
| | variable region (VH) | RIDPEDDETKYAPKFQDKVTITADTSSNTAYLQLSSLTSEDTAVFYCAG |
| | | YGNYEGAMDYWGQGTSVTVSS |
| 11 | mAb13 heavy chain | GAGGTTCTGCTGCAGCAGTCTGGGGCAGAGCTTGTGAAGCCGGGGGCCT |
| | variable region (VH) | CAGTCAGGTTGTCCTGCACAAGTTCTGACTTCAATATTAAAGACCACTT |
| | DNA sequence | TATACACTGGGTGAAGCAGAGGACTGAACAGGGCCTGGAGTGGATTGGA |
| | | AGGATTGATCCTGAAGATGATGAAACTAAATATGCCCCGAAATTCCAGG |
| | | ACAAGGTCACTATAACAGCGGACACGTCCTCCAACACAGCCTACCTGCA |
| | | GCTCAGCAGCCTGACGTCTGAGGACACTGCCGTCTTTTACTGTGCTGGT |
| | | TATGGTAACTACGAAGGGGCTATGGACTACTGGGGTCAAGGAACCTCAG |
| | | TCACCGTCTCCTCA |
| 12 | mAb13 light chain | DIVLTQSPTSLGVSPGQRATLSCRASENVNNYGIGFMNWFQQKPGQPPK |
| | variable region (VL) | LLIYAASRRGSGVPARFSGSGSGTDFSLNIHPMEEDDTAMYFCQQSKEV |
| | | PYTFGGGTNLEIR |
| 13 | mAb13 light chain | GACATTGTGCTGACCCAATCTCCAACTTCTTTGGGTGTGTCTCCAGGGC |
| | variable region (VL) | AGAGGGCCACCCTCTCCTGCAGAGCCAGCGAGAATGTTAATAATTATGG |
| | DNA sequence | CATTGGTTTTATGAACTGGTTCCAACAGAAACCAGGACAGCCACCGAAA |
| | | CTCCTCATTTATGCTGCATCCAGGCGAGGATCCGGGGTCCCTGCCAGGT |
| | | TTAGTGGCAGTGGGTCAGGGACAGACTTCAGCCTCAACATCCATCCTAT |
| | | GGAGGAGGATGATACTGCAATGTATTTCTGTCAACAAAGTAAGGAGGTT |
| | | CCGTACACGTTCGGAGGGGGGACCAACCTGGAAATAAGA |
| 14 | hz13-1 VH | EVQLVQSGAEVKKPGASVKVSCTSSDFNIKDHFIHWVRQAPGKGLEWMG |
| | | RIDPEDDETKYAPKFQDRVTMTADTSTDTAYMELSSLRSEDTAVYYCAG |
| | | YGNYEGAMDYWGQGTLVTVSS |
| 15 | hz13-1 VH DNA | GAGGTGCAGCTGGTGCAGTCCGGAGCTGAGGTGAAGAAGCCAGGAGCTT |
| | sequence | CCGTGAAGGTGAGCTGTACATCCAGCGACTTCAACATCAAGGATCACTT |
| | | TATCCATTGGGTGAGACAGGCTCCAGGCAAGGGACTGGAGTGGATGGGA |
| | | AGGATCGACCCCGAGGACGATGAGACAAAGTACGCTCCTAAGTTCCAGG |
| | | ATAGGGTGACCATGACAGCCGACACCTCTACAGATACCGCTTATATGGA |
| | | GCTGTCTTCCCTGAGATCCGAGGACACCGCCGTGTACTATTGCGCTGGC |
| | | TACGGCAATTATGAGGGCGCCATGGATTACTGGGGCCAGGGCACACTGG |
| | | TGACCGTGAGCTCT |
| 16 | Hz13-1 VL | DIQMTQSPSSLSASVGDRVTITCRASENVNNYGIGFMNWYQQKPGKAPK |
| | | LLIYAASRRGSGVPSRFSGSGSGTDFTLTISSLQPEDFATYYCQQSKEV |
| | | PYTFGQGTKVEIK |
| 17 | Hz13-1 VL DNA | GACATCCAGATGACCCAGTCTCCATCCAGCCTGTCTGCCTCCGTGGGCG |
| | sequence | ATAGAGTGACCATCACATGTCGCGCTTCCGAGAACGTGAACAATTACGG |
| | | CATCGGCTTCATGAATTGGTATCAGCAGAAGCCAGGCAAGGCCCCCAAG |
| | | CTGCTGATCTACGCTGCTAGCAGGAGGGGCTCTGGAGTGCCCTCCAGGT |
| | | TCAGCGGCTCTGGCTCCGGAACCGACTTTACCCTGACAATCTCTTCCCT |
| | | GCAGCCTGAGGATTTCGCTACATACTATTGCCAGCAGTCCAAGGAGGTG |
| | | CCATATACCTTTGGCCAGGGCACAAAGGTGGAGATCAAG |
| 18 | hz13-2 VH | EVQLVQSGAEVKKPGASVKVSCTSSDFNIKDHFIHWVRQAPGKGLEWMG |
| | | RIDPEDDETKYAPKFQDRVTMTADTSTDTAYMELSSLRSEDTAVYYCAG |
| | | YGNYEGAMDYWGQGTLVTVSS |
| 19 | hz13-2 VH DNA | GAGGTGCAGCTGGTGCAGTCCGGAGCTGAGGTGAAGAAGCCAGGAGCTT |
| | sequence | CCGTGAAGGTGAGCTGTACATCCAGCGACTTCAACATCAAGGATCACTT |
| | | TATCCATTGGGTGAGACAGGCTCCAGGCAAGGGACTGGAGTGGATGGGA |
| | | AGGATCGACCCCGAGGACGATGAGACAAAGTACGCTCCTAAGTTCCAGG |
| | | ATAGGGTGACCATGACAGCCGACACCTCTACAGATACCGCTTATATGGA |
| | | GCTGTCTTCCCTGAGATCCGAGGACACCGCCGTGTACTATTGCGCTGGC |
| | | TACGGCAATTATGAGGGCGCCATGGATTACTGGGGCCAGGGCACACTGG |
| | | TGACCGTGAGCTCT |
| 20 | hz13-2 VL | DIQMTQSPSSLSASVGDRVTITCRASENVNNYGIGFMNWFQQKPGKAPK |
| | | LLIYAASRRGSGVPSRFSGSGSGTDFTLTISSLQPEDFATYFCQQSKEV |
| | | PYTFGQGTKVEIK |
| 21 | hz13-2 VL DNA | GACATCCAGATGACCCAGTCTCCATCCAGCCTGTCTGCCTCCGTGGGCG |
| | sequence | ATAGAGTGACCATCACATGTCGCGCTTCCGAGAACGTGAACAATTACGG |
| | | CATCGGCTTCATGAATTGGTTTCAGCAGAAGCCAGGCAAGGCCCCCAAG |
| | | CTGCTGATCTATGCTGCTAGCAGGAGGGGCTCTGGAGTGCCCTCCAGGT |
| | | TCAGCGGCTCTGGCTCCGGAACCGACTTTACCCTGACAATCTCTTCCCT |
| | | GCAGCCTGAGGATTTCGCTACATACTTTTGCCAGCAGTCCAAGGAGGTG |
| | | CCATATACCTTCGGCCAGGGCACAAAGGTGGAGATCAAG |
| 22 | hz13-3 VH | EVQLVQSGAEVKKPGASVKVSCTSSDFNIKDHFIHWVRQAPGKGLEWMG |
| | | RIDPEDDETKYAPKFQDRVTMTADTSTDTAYMELSSLRSEDTAVYYCAG |
| | | YGNYEGAMDYWGQGTLVTVSS |
| 23 | hz13-3 VH DNA | GAGGTGCAGCTGGTGCAGTCCGGAGCTGAGGTGAAGAAGCCAGGAGCTT |
| | sequence | CCGTGAAGGTGAGCTGTACATCCAGCGACTTCAACATCAAGGATCACTT |
| | | TATCCATTGGGTGAGACAGGCTCCAGGCAAGGGACTGGAGTGGATGGGA |
| | | AGGATCGACCCCGAGGACGATGAGACAAAGTACGCTCCTAAGTTCCAGG |
| | | ATAGGGTGACCATGACAGCCGACACCTCTACAGATACCGCTTATATGGA |
| | | GCTGTCTTCCCTGAGATCCGAGGACACCGCCGTGTACTATTGCGCTGGC |
| | | TACGGCAATTATGAGGGCGCCATGGATTACTGGGGCCAGGGCACACTGG |
| | | TGACCGTGAGCTCT |
| 24 | hz13-3 VL | DIVMTQSPSSLSASVGDRVTLTCRASENVNNYGIGFMNWFQQKPGKAPK |
| | | LLIYAASRRGSGVPSRFSGSGSGTDFTLTISSMQPEDFATYFCQQSKEV |
| | | PYTFGQGTKLEIK |
| 25 | hz13-3 VL DNA | GACATCCAGATGACCCAGTCTCCATCCAGCCTGTCTGCCTCCGTGGGCG |
| | sequence | ATAGAGTGACCATCACATGTCGCGCTTCCGAGAACGTGAACAATTACGG |
| | | CATCGGCTTCATGAATTGGTTTCAGCAGAAGCCAGGCAAGGCCCCCAAG |
| | | CTGCTGATCTATGCTGCTAGCAGGAGGGGCTCTGGAGTGCCCTCCAGGT |
| | | TCAGCGGCTCTGGCTCCGGAACCGACTTTACCCTGACAATCTCTTCCCT |
| | | GCAGCCTGAGGATTTCGCTACATACTTTTGCCAGCAGTCCAAGGAGGTG |
| | | CCATATACCTTCGGCCAGGGCACAAAGGTGGAGATCAAG |
| 26 | hz13-4 VH | EVLLVQSGAEVKKPGASVKLSCTSSDFNIKDHFIHWVRQAPGKGLEWMG |
| | | RIDPEDDETKYAPKFQDRVTMTADTSTDTAYMELSSLRSEDTAVFYCAG |
| | | YGNYEGAMDYWGQGTLVTVSS |
| 27 | hz13-4 VH DNA | GAGGTGCTGCTGGTGCAGTCCGGAGCTGAGGTGAAGAAGCCAGGAGCTT |
| | sequence | CCGTGAAGCTGAGCTGTACATCCAGCGACTTCAACATCAAGGATCACTT |
| | | TATCCATTGGGTGAGACAGGCTCCAGGCAAGGGACTGGAGTGGATGGGA |
| | | AGGATCGACCCCGAGGACGATGAGACAAAGTACGCTCCTAAGTTCCAGG |
| | | ATAGGGTGACCATGACAGCCGACACCTCTACAGATACCGCTTATATGGA |
| | | GCTGTCTTCCCTGAGATCCGAGGACACCGCCGTGTTTTACTGCGCTGGC |
| | | TACGGCAATTATGAGGGCGCCATGGATTATTGGGGCCAGGGCACACTGG |
| | | TGACCGTGAGCTCT |
| 28 | Hz13-4 VL | DIQMTQSPSSLSASVGDRVTITCRASENVNNYGIGFMNWYQQKPGKAPK |
| | | LLIYAASRRGSGVPSRFSGSGSGTDFTLTISSLQPEDFATYYCQQSKEV |
| | | PYTFGQGTKVEIK |
| 29 | Hz13-4 VL DNA | GACATCCAGATGACCCAGTCTCCATCCAGCCTGTCTGCCTCCGTGGGCG |
| | sequence | ATAGAGTGACCATCACATGTCGCGCTTCCGAGAACGTGAACAATTACGG |
| | | CATCGGCTTCATGAATTGGTATCAGCAGAAGCCAGGCAAGGCCCCCAAG |
| | | CTGCTGATCTACGCTGCTAGCAGGAGGGGCTCTGGAGTGCCCTCCAGGT |
| | | TCAGCGGCTCTGGCTCCGGAACCGACTTTACCCTGACAATCTCTTCCCT |
| | | GCAGCCTGAGGATTTCGCTACATACTATTGCCAGCAGTCCAAGGAGGTG |
| | | CCATATACCTTTGGCCAGGGCACAAAGGTGGAGATCAAG |
| 30 | hz13-5 VH | EVLLVQSGAEVKKPGASVKLSCTSSDFNIKDHFIHWVRQAPGKGLEWMG |
| | | RIDPEDDETKYAPKFQDRVTMTADTSTDTAYMELSSLRSEDTAVFYCAG |
| | | YGNYEGAMDYWGQGTLVTVSS |
| 31 | hz13-5 VH DNA | GAGGTGCTGCTGGTGCAGTCCGGAGCTGAGGTGAAGAAGCCAGGAGCTT |
| | sequence | CCGTGAAGCTGAGCTGTACATCCAGCGACTTCAACATCAAGGATCACTT |
| | | TATCCATTGGGTGAGACAGGCTCCAGGCAAGGGACTGGAGTGGATGGGA |
| | | AGGATCGACCCCGAGGACGATGAGACAAAGTACGCTCCTAAGTTCCAGG |
| | | ATAGGGTGACCATGACAGCCGACACCTCTACAGATACCGCTTATATGGA |
| | | GCTGTCTTCCCTGAGATCCGAGGACACCGCCGTGTTTTACTGCGCTGGC |
| | | TACGGCAATTATGAGGGCGCCATGGATTATTGGGGCCAGGGCACACTGG |
| | | TGACCGTGAGCTCT |
| 32 | hz13-5 VL | DIQMTQSPSSLSASVGDRVTITCRASENVNNYGIGFMNWFQQKPGKAPK |
| | | LLIYAASRRGSGVPSRFSGSGSGTDFTLTISSLQPEDFATYFCQQSKEV |
| | | PYTFGQGTKVEIK |
| 33 | hz13-5 VL DNA | GACATCCAGATGACCCAGTCTCCATCCAGCCTGTCTGCCTCCGTGGGCG |
| | sequence | ATAGAGTGACCATCACATGTCGCGCTTCCGAGAACGTGAACAATTACGG |
| | | CATCGGCTTCATGAATTGGTTTCAGCAGAAGCCAGGCAAGGCCCCCAAG |
| | | CTGCTGATCTATGCTGCTAGCAGGAGGGGCTCTGGAGTGCCCTCCAGGT |
| | | TCAGCGGCTCTGGCTCCGGAACCGACTTTACCCTGACAATCTCTTCCCT |
| | | GCAGCCTGAGGATTTCGCTACATACTTTTGCCAGCAGTCCAAGGAGGTG |
| | | CCATATACCTTCGGCCAGGGCACAAAGGTGGAGATCAAG |
| 34 | hz13-6 VH | EVLLVQSGAEVKKPGASVKLSCTSSDFNIKDHFIHWVRQAPGKGLEWMG |
| | | RIDPEDDETKYAPKFQDRVTMTADTSTDTAYMELSSLRSEDTAVFYCAG |
| | | YGNYEGAMDYWGQGTLVTVSS |
| 35 | hz13-6 VH DNA | GAGGTGCTGCTGGTGCAGTCCGGAGCTGAGGTGAAGAAGCCAGGAGCTT |
| | sequence | CCGTGAAGCTGAGCTGTACATCCAGCGACTTCAACATCAAGGATCACTT |
| | | TATCCATTGGGTGAGACAGGCTCCAGGCAAGGGACTGGAGTGGATGGGA |
| | | AGGATCGACCCCGAGGACGATGAGACAAAGTACGCTCCTAAGTTCCAGG |
| | | ATAGGGTGACCATGACAGCCGACACCTCTACAGATACCGCTTATATGGA |
| | | GCTGTCTTCCCTGAGATCCGAGGACACCGCCGTGTTTTACTGCGCTGGC |
| | | TACGGCAATTATGAGGGCGCCATGGATTATTGGGGCCAGGGCACACTGG |
| | | TGACCGTGAGCTCT |
| 36 | hz13-6 VL | DIVMTQSPSSLSASVGDRVTLTCRASENVNNYGIGFMNWFQQKPGKAPK |
| | | LLIYAASRRGSGVPSRFSGSGSGTDFTLTISSMQPEDFATYFCQQSKEV |
| | | PYTFGQGTKLEIK |
| 37 | hz13-6 VL DNA | GACATCGTGATGACCCAGTCTCCATCCAGCCTGTCTGCCTCCGTGGGCG |
| | sequence | ATAGAGTGACCCTGACATGTCGCGCTTCCGAGAACGTGAACAATTACGG |
| | | CATCGGCTTCATGAATTGGTTTCAGCAGAAGCCAGGCAAGGCCCCCAAG |
| | | CTGCTGATCTATGCTGCTAGCAGGAGGGGCTCTGGAGTGCCCTCCAGGT |
| | | TCAGCGGCTCTGGCTCCGGAACCGACTTTACCCTGACAATCTCTTCCAT |
| | | GCAGCCTGAGGATTTCGCTACATACTTTTGCCAGCAGTCCAAGGAGGTG |
| | | CCATATACCTTCGGCCAGGGCACAAAGCTGGAGATCAAG |
| 38 | hz13-7 VH | EVLLVQSGAEVKKPGASVKLSCTSSDFNIKDHFIHWVRQAPGKGLEWIG |
| | | RIDPEDDETKYAPKFQDRVTITADTSTDTAYLELSSLRSEDTAVFYCAG |
| | | YGNYEGAMDYWGQGTLVTVSS |
| 39 | hz137-VH DNA | GAGGTGCTGCTGGTGCAGTCCGGAGCTGAGGTGAAGAAGCCAGGAGCTT |
| | sequence | CCGTGAAGCTGAGCTGTACATCCAGCGACTTCAACATCAAGGATCACTT |
| | | TATCCATTGGGTGAGACAGGCTCCAGGCAAGGGACTGGAGTGGATCGGA |
| | | AGGATCGACCCCGAGGACGATGAGACAAAGTACGCTCCTAAGTTCCAGG |
| | | ATAGGGTGACCATCACAGCCGACACCTCTACAGATACCGCTTATCTGGA |
| | | GCTGTCTTCCCTGAGATCCGAGGACACCGCCGTGTTTTACTGCGCTGGC |
| | | TACGGCAATTATGAGGGCGCCATGGATTATTGGGGCCAGGGCACACTGG |
| | | TGACCGTGAGCTCT |
| 40 | Hz13-7 VL | DIQMTQSPSSLSASVGDRVTITCRASENVNNYGIGFMNWYQQKPGKAPK |
| | | LLIYAASRRGSGVPSRFSGSGSGTDFTLTISSLQPEDFATYYCQQSKEV |
| | | PYTFGQGTKVEIK |
| 41 | Hz13-7 VL DNA | GACATCCAGATGACCCAGTCTCCATCCAGCCTGTCTGCCTCCGTGGGCG |
| | sequence | ATAGAGTGACCATCACATGTCGCGCTTCCGAGAACGTGAACAATTACGG |
| | | CATCGGCTTCATGAATTGGTATCAGCAGAAGCCAGGCAAGGCCCCCAAG |
| | | CTGCTGATCTACGCTGCTAGCAGGAGGGGCTCTGGAGTGCCCTCCAGGT |
| | | TCAGCGGCTCTGGCTCCGGAACCGACTTTACCCTGACAATCTCTTCCCT |
| | | GCAGCCTGAGGATTTCGCTACATACTATTGCCAGCAGTCCAAGGAGGTG |
| | | CCATATACCTTTGGCCAGGGCACAAAGGTGGAGATCAAG |
| 42 | hz13-8 VH | EVLLVQSGAEVKKPGASVKLSCTSSDFNIKDHFIHWVRQAPGKGLEWIG |
| | | RIDPEDDETKYAPKFQDRVTITADTSTDTAYLELSSLRSEDTAVFYCAG |
| | | YGNYEGAMDYWGQGTLVTVSS |
| 43 | hz13-8 VH DNA | GAGGTGCTGCTGGTGCAGTCCGGAGCTGAGGTGAAGAAGCCAGGAGCTT |
| | sequence | CCGTGAAGCTGAGCTGTACATCCAGCGACTTCAACATCAAGGATCACTT |
| | | TATCCATTGGGTGAGACAGGCTCCAGGCAAGGGACTGGAGTGGATCGGA |
| | | AGGATCGACCCCGAGGACGATGAGACAAAGTACGCTCCTAAGTTCCAGG |
| | | ATAGGGTGACCATCACAGCCGACACCTCTACAGATACCGCTTATCTGGA |
| | | GCTGTCTTCCCTGAGATCCGAGGACACCGCCGTGTTTTACTGCGCTGGC |
| | | TACGGCAATTATGAGGGCGCCATGGATTATTGGGGCCAGGGCACACTGG |
| | | TGACCGTGAGCTCT |
| 44 | hz13-8 VL | DIQMTQSPSSLSASVGDRVTITCRASENVNNYGIGFMNWFQQKPGKAPK |
| | | LLIYAASRRGSGVPSRFSGSGSGTDFTLTISSLQPEDFATYFCQQSKEV |
| | | PYTFGQGTKVEIK |
| 45 | hz13-8 VL DNA | GACATCCAGATGACCCAGTCTCCATCCAGCCTGTCTGCCTCCGTGGGCG |
| | sequence | ATAGAGTGACCATCACATGTCGCGCTTCCGAGAACGTGAACAATTACGG |
| | | CATCGGCTTCATGAATTGGTTTCAGCAGAAGCCAGGCAAGGCCCCCAAG |
| | | CTGCTGATCTATGCTGCTAGCAGGAGGGGCTCTGGAGTGCCCTCCAGGT |
| | | TCAGCGGCTCTGGCTCCGGAACCGACTTTACCCTGACAATCTCTTCCCT |
| | | GCAGCCTGAGGATTTCGCTACATACTTTTGCCAGCAGTCCAAGGAGGTG |
| | | CCATATACCTTCGGCCAGGGCACAAAGGTGGAGATCAAG |
| 46 | hz13-9 VH | EVLLVQSGAEVKKPGASVKLSCTSSDFNIKDHFIHWVRQAPGKGLEWIG |
| | | RIDPEDDETKYAPKFQDRVTITADTSTDTAYLELSSLRSEDTAVFYCAG |
| | | YGNYEGAMDYWGQGTLVTVSS |
| 47 | hz13-9 VH DNA | GAGGTGCTGCTGGTGCAGTCCGGAGCTGAGGTGAAGAAGCCAGGAGCTT |
| | sequence | CCGTGAAGCTGAGCTGTACATCCAGCGACTTCAACATCAAGGATCACTT |
| | | TATCCATTGGGTGAGACAGGCTCCAGGCAAGGGACTGGAGTGGATCGGA |
| | | AGGATCGACCCCGAGGACGATGAGACAAAGTACGCTCCTAAGTTCCAGG |
| | | ATAGGGTGACCATCACAGCCGACACCTCTACAGATACCGCTTATCTGGA |
| | | GCTGTCTTCCCTGAGATCCGAGGACACCGCCGTGTTTTACTGCGCTGGC |
| | | TACGGCAATTATGAGGGCGCCATGGATTATTGGGGCCAGGGCACACTGG |
| | | TGACCGTGAGCTCT |
| 48 | hz13-9 VL | DIVMTQSPSSLSASVGDRVTLTCRASENVNNYGIGFMNWFQQKPGKAPK |
| | | LLIYAASRRGSGVPSRFSGSGSGTDFTLTISSMQPEDFATYFCQQSKEV |
| | | PYTFGQGTKLEIK |
| 49 | hz13-9 VL DNA | GACATCGTGATGACCCAGTCTCCATCCAGCCTGTCTGCCTCCGTGGGCG |
| | sequence | ATAGAGTGACCCTGACATGTCGCGCTTCCGAGAACGTGAACAATTACGG |
| | | CATCGGCTTCATGAATTGGTTTCAGCAGAAGCCAGGCAAGGCCCCCAAG |
| | | CTGCTGATCTATGCTGCTAGCAGGAGGGGCTCTGGAGTGCCCTCCAGGT |
| | | TCAGCGGCTCTGGCTCCGGAACCGACTTTACCCTGACAATCTCTTCCAT |
| | | GCAGCCTGAGGATTTCGCTACATACTTTTGCCAGCAGTCCAAGGAGGTG |
| | | CCATATACCTTCGGCCAGGGCACAAAGCTGGAGATCAAG |
| 50 | hz13-10 VH | EVQLVQSGAEVKKPGATVKISCKVSDFNIKDHFIHWVQQAPGKGLEWMG |
| | | RIDPEDDETKYAPKFQDRVTITADTSTDTAYMELSSLRSEDTAVYYCAG |
| | | YGNYEGAMDYWGQGTTVTVSS |
| 51 | hz13-10 VH DNA | GAGGTGCAGCTGGTGCAGAGCGGAGCTGAGGTGAAGAAGCCAGGAGCTA |
| | sequence | CCGTGAAGATCTCCTGTAAGGTGAGCGACTTCAACATCAAGGATCACTT |
| | | TATCCATTGGGTGCAGCAGGCTCCTGGCAAGGGCCTGGAGTGGATGGGC |
| | | AGAATCGACCCCGAGGACGATGAGACCAAGTACGCTCCTAAGTTCCAGG |
| | | ATCGCGTGACCATCACAGCCGACACCTCTACAGATACCGCTTATATGGA |
| | | GCTGTCCAGCCTGAGATCCGAGGACACAGCCGTGTACTATTGCGCTGGC |
| | | TACGGCAATTATGAGGGCGCCATGGATTACTGGGGCCAGGGCACCACAG |
| | | TGACAGTGTCTTCC |
| 52 | hz13-10 VL | DVVMTQSPLSLPVTLGQPASISCRASENVNNYGIGFMNWFQQRPGQSPR |
| | | LLIYAASRRGSGVPDRFSGSGSGTDFTLKISRVEEEDVGVYYCQQSKEV |
| | | PYTFGGGTKVEIK |
| 53 | hz13-10 VL DNA | GATGTTGTGATGACTCAGTCTCCACTCTCCCTGCCCGTCACCCTTGGAC |
| | sequence | AGCCGGCCTCCATCTCCTGCCGCGCTTCCGAGAATGTGAACAATTACGG |
| | | CATCGGCTTCATGAACTGGTTTCAGCAGAGGCCAGGCCAATCTCCAAGG |
| | | CTGCTAATTTATGCTGCTTCTAGGAGGGGCTCCGGGGTCCCAGACAGAT |
| | | TCAGCGGCAGTGGGTCAGGCACTGATTTCACACTGAAAATCAGCAGGGT |
| | | GGAGGAAGAGGATGTTGGGGTTTATTACTGCCAGCAGTCCAAGGAGGTG |
| | | CCCTATACCTTTGGCGGCGGCACAAAGGTGGAGATCAAG |
| 54 | hz13-11 VH | EVQLVQSGAEVKKPGATVKISCTSSDFNIKDHFIHWVQQAPGKGLEWMG |
| | | RIDPEDDETKYAPKFQDRVTITADTSTDTAYMELSSLRSEDTAVYYCAG |
| | | YGNYEGAMDYWGQGTTVTVSS |
| 55 | hz13-11 VH DNA | GAGGTGCAGCTGGTGCAGAGCGGAGCTGAGGTGAAGAAGCCAGGAGCTA |
| | sequence | CCGTGAAGATCTCCTGTACCAGCAGCGACTTCAACATCAAGGATCACTT |
| | | TATCCATTGGGTGCAGCAGGCTCCTGGCAAGGGCCTGGAGTGGATGGGC |
| | | AGAATCGACCCCGAGGACGATGAGACCAAGTACGCTCCTAAGTTCCAGG |
| | | ATCGCGTGACCATCACAGCCGACACCTCTACAGATACCGCTTATATGGA |
| | | GCTGTCCAGCCTGAGATCCGAGGACACAGCCGTGTACTATTGCGCTGGC |
| | | TACGGCAATTATGAGGGCGCCATGGATTACTGGGGCCAGGGCACCACAG |
| | | TGACAGTGTCTTCC |
| 56 | hz13-11 VL | DVVMTQSPLSLPVTLGQPASISCRASENVNNYGIGFMNWFQQRPGQSPR |
| | | RLIYAASRRGSGVPDRFSGSGSGTDFTLKISRVEAEDVGVYYCQQSKEV |
| | | PYTFGGGTKVEIK |
| 57 | hz13-11 VL DNA | GATGTTGTGATGACTCAGTCTCCACTCTCCCTGCCCGTCACCCTTGGAC |
| | sequence | AGCCGGCCTCCATCTCCTGCCGCGCTTCCGAGAATGTGAACAATTACGG |
| | | CATCGGCTTCATGAACTGGTTTCAGCAGAGGCCAGGCCAATCTCCAAGG |
| | | CGCCTAATTTATGCTGCTTCTAGGAGGGGCTCCGGGGTCCCAGACAGAT |
| | | TCAGCGGCAGTGGGTCAGGCACTGATTTCACACTGAAAATCAGCAGGGT |
| | | GGAGGCTGAGGATGTTGGGGTTTATTACTGCCAGCAGTCCAAGGAGGTG |
| | | CCCTATACCTTTGGCGGCGGCACAAAGGTGGAGATCAAG |
| 58 | hz13-12 VH | EVQLVQSGAEVKKPGATVKISCTSSDFNIKDHFIHWVQQAPGKGLEWMG |
| | | RIDPEDDETKYAPKFQDRVTITADTSTDTAYMELSSLRSEDTAVYYCAG |
| | | YGNYEGAMDYWGQGTTVTVSS |
| 59 | hz13-12 VH DNA | GAGGTGCAGCTGGTGCAGAGCGGAGCTGAGGTGAAGAAGCCAGGAGCTA |
| | sequence | CCGTGAAGATCTCCTGTACCAGCAGCGACTTCAACATCAAGGATCACTT |
| | | TATCCATTGGGTGCAGCAGGCTCCTGGCAAGGGCCTGGAGTGGATGGGC |
| | | AGAATCGACCCCGAGGACGATGAGACCAAGTACGCTCCTAAGTTCCAGG |
| | | ATCGCGTGACCATCACAGCCGACACCTCTACAGATACCGCTTATATGGA |
| | | GCTGTCCAGCCTGAGATCCGAGGACACAGCCGTGTACTATTGCGCTGGC |
| | | TACGGCAATTATGAGGGCGCCATGGATTACTGGGGCCAGGGCACCACAG |
| | | TGACAGTGTCTTCC |
| 60 | hz13-12 VL | DVVMTQSPLSLPVTLGQPASISCRASENVNNYGIGFMNWFQQRPGQSPR |
| | | LLIYAASRRGSGVPDRFSGSGSGTDFTLKISRVEEEDVGVYYCQQSKEV |
| | | PYTFGGGTKVEIK |
| 61 | hz13-12 VL DNA | GATGTTGTGATGACTCAGTCTCCACTCTCCCTGCCCGTCACCCTTGGAC |
| | sequence | AGCCGGCCTCCATCTCCTGCCGCGCTTCCGAGAATGTGAACAATTACGG |
| | | CATCGGCTTCATGAACTGGTTTCAGCAGAGGCCAGGCCAATCTCCAAGG |
| | | CTGCTAATTTATGCTGCTTCTAGGAGGGGCTCCGGGGTCCCAGACAGAT |
| | | TCAGCGGCAGTGGGTCAGGCACTGATTTCACACTGAAAATCAGCAGGGT |
| | | GGAGGAAGAGGATGTTGGGGTTTATTACTGCCAGCAGTCCAAGGAGGTG |
| | | CCCTATACCTTTGGCGGCGGCACAAAGGTGGAGATCAAG |
| 62 | HCDR1 hz13-5 D31E | DFNIKEHFIH |
| | and hz13-12 D31E | |
| 63 | HCDR2 hz13-5 D31E | RIDPEDDETKYAPKFQD |
| | and hz13-12 D31E | |
| 64 | HCDR3 hz13-5 D31E | YGNYEGAMDY |
| | and hz13-12 D31E | |
| 65 | LCDR1 hz13-5 D31E | RASENVNNYGIGFMN |
| | and hz13-12 D31E | |
| 66 | LCDR2 hz13-5 D31E | AASRRGS |
| | and hz13-12 D31E | |
| 67 | LCDR3 hz13-5 D31E | QQSKEVPYT |
| | and hz13-12 D31E | |
| 68 | hz13-5 D31E VH | EVLLVQSGAEVKKPGASVKLSCTSSDFNIKEHFIHWVRQAPGKGLEWMG |
| | | RIDPEDDETKYAPKFQDRVTMTADTSTDTAYMELSSLRSEDTAVFYCAG |
| | | YGNYEGAMDYWGQGTLVTVSS |
| 69 | hz13-5 D31E VH DNA | GAAGTGCTACTAGTGCAAAGTGGTGCAGAAGTCAAGAAGCCTGGAGCTT |
| | sequence | CCGTGAAGCTGTCCTGCACCAGCTCCGACTTCAACATCAAGGAACACTT |
| | | CATCCACTGGGTGCGGCAGGCCCCCGGCAAGGGCCTGGAATGGATGGGC |
| | | AGAATCGATCCTGAGGACGACGAGACCAAGTACGCCCCTAAATTTCAGG |
| | | ACAGAGTGACCATGACAGCTGATACCAGCACCGACACCGCCTACATGGA |
| | | ACTGTCTTCTCTGAGATCCGAGGATACAGCCGTGTTCTACTGTGCTGGA |
| | | TATGGCAACTACGAGGGCGCCATGGACTACTGGGGCCAGGGCACCCTGG |
| | | TGACCGTGTCCTCT |
| 70 | hz13-5 D31E VL | DIQMTQSPSSLSASVGDRVTITCRASENVNNYGIGFMNWFQQKPGKAPK |
| | | LLIYAASRRGSGVPSRFSGSGSGTDFTLTISSLQPEDFATYFCQQSKEV |
| | | PYTFGQGTKVEIK |
| 71 | hz13-5 D31E VL DNA | GATATCCAAATGACTCAAAGTCCAAGTAGTCTGTCTGCTTCCGTGGGCG |
| | sequence | ACAGAGTGACCATCACCTGTAGAGCCTCCGAGAATGTGAACAACTACGG |
| | | CATCGGCTTCATGAACTGGTTCCAGCAGAAGCCTGGCAAGGCCCCTAAG |
| | | CTGCTGATCTACGCCGCTTCTCGGAGAGGCTCCGGCGTGCCCAGCCGGT |
| | | TCTCCGGATCTGGCTCTGGAACCGACTTCACCCTGACAATCTCCTCCCT |
| | | GCAGCCTGAGGATTTTGCCACCTACTTCTGCCAGCAAAGCAAAGAGGTT |
| | | CCATACACCTTCGGCCAGGGCACCAAGGTGGAAATCAAG |
| 72 | HCDR1 mAb20 | GYTFTNYGIS |
| 73 | HCDR2 mAb20 | EIYPRSGNTYHNEKFKD |
| 74 | HCDR3 mAb20 | SMITTRYWYFDV |
| 75 | LCDR1 mAb20 | RASESVDTYGNSFMH |
| 76 | LCDR2 mAb20 | LASNLES |
| 77 | LCDR3 mAb20 | QQNNEDPLT |
| 78 | mAb20 VH | QVQLQQSGAELARPGASVKLSCKATGYTFTNYGISWVKQRTGQGLEWIG |
| | | EIYPRSGNTYHNEKFKDKATLTADKSSSTAYMEFRSLTSEDSAVYFCAR |
| | | SMITTRYWYFDVWGKGTTVTVSS |
| 79 | mAb20 VH DNA | CAGGTTCAGCTGCAGCAGTCTGGAGCTGAGCTGGCGAGGCCTGGGGCTT |
| | sequence | CAGTGAAGCTGTCCTGCAAGGCTACTGGCTACACCTTCACAAACTATGG |
| | | AATAAGCTGGGTGAAGCAGAGAACTGGACAGGGCCTTGAGTGGATTGGA |
| | | GAGATTTATCCTAGAAGTGGTAATACTTACCACAATGAGAAGTTCAAGG |
| | | ACAAGGCCACACTGACTGCAGACAAATCCTCCAGCACAGCGTACATGGA |
| | | ATTCCGCAGCCTGACATCTGAGGACTCTGCGGTCTATTTCTGTGCAAGA |
| | | TCGATGATTACGACGAGGTACTGGTACTTCGATGTCTGGGGCAAAGGGA |
| | | CCACGGTCACCGTCTCCTCA |
| 80 | mAb20 VL | NIVLTQSPASLAVSLGQRATLSCRASESVDTYGNSFMHWYQQKPGQPPK |
| | | LLIYLASNLESGVPARFSGSGSRTDFTLTIDPVEADDAATYYCQQNNED |
| | | PLTFGAGTKLELK |
| 81 | mAb20 VL DNA | AACATTGTGCTGACCCAATCTCCAGCTTCTTTGGCTGTGTCTCTAGGGC |
| | sequence | AGAGGGCCACCTTATCCTGCAGAGCCAGTGAAAGTGTTGATACTTATGG |
| | | CAATAGTTTTATGCACTGGTACCAGCAGAAACCAGGTCAGCCACCCAAA |
| | | CTCCTCATCTATCTTGCATCCAACCTAGAATCTGGGGTCCCTGCCAGGT |
| | | TCAGTGGCAGTGGGTCTAGGACAGACTTCACCCTCACCATTGATCCTGT |
| | | GGAGGCTGATGATGCTGCAACCTATTACTGTCAGCAAAATAATGAGGAT |
| | | CCGCTCACGTTCGGTGCTGGGACCAAGCTGGAGCTGAAA |
| 82 | hz20-1 VH | QVQLVQSGAEVKKPGASVKVSCKASGYTFTNYGISWVRQAPGQGLEWMG |
| | | EIYPRSGNTYHNEKFKDRVTMTRDTSTSTVYMELSSLRSEDTAVYYCAR |
| | | SMITTRYWYFDVWGKGTTVTVSS |
| 83 | hz20-1 VH DNA | CAGGTGCAGCTGGTGCAGAGCGGCGCCGAGGTGAAGAAGCCCGGCGCCA |
| | sequence | GCGTGAAGGTGAGCTGCAAGGCCAGCGGCTACACCTTCACCAACTACGG |
| | | CATCAGCTGGGTGAGGCAGGCCCCCGGCCAGGGCCTGGAGTGGATGGGC |
| | | GAGATCTACCCCAGGAGCGGCAACACCTACCACAACGAGAAGTTCAAGG |
| | | ACAGGGTGACCATGACCAGGGACACCAGCACCAGCACCGTGTACATGGA |
| | | GCTGAGCAGCCTGAGGAGCGAGGACACCGCCGTGTACTACTGCGCCAGG |
| | | AGCATGATCACCACCAGGTACTGGTACTTCGACGTGTGGGGCAAGGGCA |
| | | CCACCGTGACCGTGAGCAGC |
| 84 | hz20-1 VL | DIVMTQSPDSLAVSLGERATINCRASESVDTYGNSFMHWYQQKPGQPPK |
| | | LLIYLASNLESGVPDRFSGSGSGTDFTLTISSLQAEDVAVYYCQQNNED |
| | | PLTFGAGTKLELK |
| 85 | hz20-1 VL DNA | GACATCGTGATGACCCAGTCTCCCGATTCCCTGGCCGTGAGCCTGGGAG |
| | sequence | AGAGGGCTACCATCAACTGTCGGGCTTCCGAGAGCGTGGACACATACGG |
| | | CAATTCTTTCATGCACTGGTATCAGCAGAAGCCCGGCCAGCCCCCTAAG |
| | | CTGCTGATCTACCTGGCTAGCAACCTGGAGTCTGGCGTGCCTGACAGAT |
| | | TCTCTGGCTCCGGCAGCGGCACCGACTTCACCCTGACAATCTCCAGCCT |
| | | GCAGGCCGAGGACGTGGCCGTGTACTATTGCCAGCAGAACAATGAGGAC |
| | | CCCCTGACCTTCGGCGCTGGCACAAAGCTGGAGCTGAAG |
| 86 | hz20-2 VH | QVQLVQSGAEVKKPGASVKVSCKASGYTFTNYGISWVRQAPGQGLEWMG |
| | | EIYPRSGNTYHNEKFKDRVTMTTDTSTSTAYMELRSLRSDDTAVYYCAR |
| | | SMITTRYWYFDVWGQGTLVTVSS |
| 87 | hz20-2 VH DNA | CAGGTGCAGCTGGTGCAGAGCGGCGCCGAGGTGAAGAAGCCCGGCGCCA |
| | sequence | GCGTGAAGGTGAGCTGCAAGGCCAGCGGCTACACCTTCACCAACTACGG |
| | | CATCAGCTGGGTGAGGCAGGCCCCCGGCCAGGGCCTGGAGTGGATGGGC |
| | | GAGATCTACCCCAGGAGCGGCAACACCTACCACAACGAGAAGTTCAAGG |
| | | ACAGGGTGACCATGACCACCGACACCAGCACCAGCACCGCCTACATGGA |
| | | GCTGAGGAGCCTGAGGAGCGACGACACCGCCGTGTACTACTGCGCCAGG |
| | | AGCATGATCACCACCAGGTACTGGTACTTCGACGTGTGGGGCCAGGGCA |
| | | CCCTGGTGACCGTGAGCAGC |
| 88 | hz20-2 VL | DIQMTQSPSSLSASVGDRVTITCRASESVDTYGNSFMHWYQQKPGKAPK |
| | | LLIYLASNLESGVPSRFSGSGSGTDFTLTISSLQPEDFATYYCQQNNED |
| | | PLTFGQGTKVEIK |
| 89 | hz20-2 VL DNA | GATATCCAGATGACCCAGTCCCCATCCAGCCTGTCTGCCTCCGTGGGCG |
| | sequence | ATAGGGTGACCATCACATGTCGGGCTAGCGAGTCTGTGGACACCTACGG |
| | | CAACAGCTTCATGCACTGGTATCAGCAGAAGCCAGGCAAGGCCCCCAAG |
| | | CTGCTGATCTACCTGGCTTCCAATCTGGAGAGCGGCGTGCCCTCTAGAT |
| | | TCTCCGGCAGCGGCTCTGGCACCGACTTTACCCTGACAATCTCTTCCCT |
| | | GCAGCCTGAGGATTTCGCCACATACTATTGCCAGCAGAACAATGAGGAC |
| | | CCACTGACCTTTGGCCAGGGCACAAAGGTGGAGATCAAG |
| 90 | hz20-3 VH | EVQLVQSGAEVKKPGASVKVSCKASGYTFTNYGISWVRQAPGQGLEWMG |
| | | EIYPRSGNTYHNEKFKDRVTMTADKSTSTAYMELRSLRSDDTAVYFCAR |
| | | SMITTRYWYFDVWGQGTLVTVSS |
| 91 | hz20-3 VH DNA | GAGGTGCAGCTGGTGCAGAGCGGCGCCGAGGTGAAGAAGCCCGGCGCCA |
| | sequence | GCGTGAAGGTGAGCTGCAAGGCCAGCGGCTACACCTTCACCAACTACGG |
| | | CATCAGCTGGGTGAGGCAGGCCCCCGGCCAGGGCCTGGAGTGGATGGGC |
| | | GAGATCTACCCCAGGAGCGGCAACACCTACCACAACGAGAAGTTCAAGG |
| | | ACAGGGTGACCATGACCGCCGACAAGAGCACCAGCACCGCCTACATGGA |
| | | GCTGAGGAGCCTGAGGAGCGACGACACCGCCGTGTACTTCTGCGCCAGG |
| | | AGCATGATCACCACCAGGTACTGGTACTTCGACGTGTGGGGCCAGGGCA |
| | | CCCTGGTGACCGTGAGCAGC |
| 92 | hz20-3 VL | DIQMTQSPSSLSASVGDRVTITCRASESVDTYGNSFMHWYQQKPGKAPK |
| | | LLIYLASNLESGVPSRFSGSGSGTDFTLTISSLQPEDFATYYCQQNNED |
| | | PLTFGQGTKVEIK |
| 93 | hz20-3 VLDNA | GATATCCAGATGACCCAGTCCCCATCCAGCCTGTCTGCCTCCGTGGGCG |
| | sequence | ATAGGGTGACCATCACATGTCGGGCTAGCGAGTCTGTGGACACCTACGG |
| | | CAACAGCTTCATGCACTGGTATCAGCAGAAGCCAGGCAAGGCCCCCAAG |
| | | CTGCTGATCTACCTGGCTTCCAATCTGGAGAGCGGCGTGCCCTCTAGAT |
| | | TCTCCGGCAGCGGCTCTGGCACCGACTTTACCCTGACAATCTCTTCCCT |
| | | GCAGCCTGAGGATTTCGCCACATACTATTGCCAGCAGAACAATGAGGAC |
| | | CCACTGACCTTTGGCCAGGGCACAAAGGTGGAGATCAAG |
| 94 | hz20-4 VH | QVQLVQSGAEVKKPGASVKVSCKASGYTFTNYGISWVRQAPGQGLEWMG |
| | | EIYPRSGNTYHNEKFKDRVTMTADTSTSTVYMELSSLRSEDTAVYYCAR |
| | | SMITTRYWYFDVWGKGTTVTVSS |
| 95 | hz20-4 VH DNA | CAGGTGCAGCTGGTGCAGAGCGGCGCCGAGGTGAAGAAGCCCGGCGCCA |
| | sequence | GCGTGAAGGTGAGCTGCAAGGCCAGCGGCTACACCTTCACCAACTACGG |
| | | CATCAGCTGGGTGAGGCAGGCCCCCGGCCAGGGCCTGGAGTGGATGGGC |
| | | GAGATCTACCCCAGGAGCGGCAACACCTACCACAACGAGAAGTTCAAGG |
| | | ACAGGGTGACCATGACCGCCGACACCAGCACCAGCACCGTGTACATGGA |
| | | GCTGAGCAGCCTGAGGAGCGAGGACACCGCCGTGTACTACTGCGCCAGG |
| | | AGCATGATCACCACCAGGTACTGGTACTTCGACGTGTGGGGCAAGGGCA |
| | | CCACCGTGACCGTGAGCAGC |
| 96 | hz20-4 VL | DIVMTQSPDSLAVSLGERATINCRASESVDTYGNSFMHWYQQKPGQPPK |
| | | LLIYLASNLESGVPDRFSGSGSGTDFTLTISSLQAEDVAVYYCQQNNED |
| | | PLTFGAGTKLELK |
| 97 | hz20-4 VL DNA | GACATCGTGATGACCCAGTCTCCCGATTCCCTGGCCGTGAGCCTGGGAG |
| | sequence | AGAGGGCTACCATCAACTGTCGGGCTTCCGAGAGCGTGGACACATACGG |
| | | CAATTCTTTCATGCACTGGTATCAGCAGAAGCCCGGCCAGCCCCCTAAG |
| | | CTGCTGATCTACCTGGCTAGCAACCTGGAGTCTGGCGTGCCTGACAGAT |
| | | TCTCTGGCTCCGGCAGCGGCACCGACTTCACCCTGACAATCTCCAGCCT |
| | | GCAGGCCGAGGACGTGGCCGTGTACTATTGCCAGCAGAACAATGAGGAC |
| | | CCCCTGACCTTCGGCGCTGGCACAAAGCTGGAGCTGAAG |
| 98 | hz20-5 VH | QVQLVQSGAEVKKPGASVKVSCKASGYTFTNYGISWVRQRPGQGLEWIG |
| | | EIYPRSGNTYHNEKFKDRVTMTADTSTSTVYMELSSLRSEDTAVYYCAR |
| | | SMITTRYWYFDVWGKGTTVTVSS |
| 99 | hz20-5 VH DNA | CAGGTGCAGCTGGTGCAGAGCGGCGCCGAGGTGAAGAAGCCCGGCGCCA |
| | sequence | GCGTGAAGGTGAGCTGCAAGGCCAGCGGCTACACCTTCACCAACTACGG |
| | | CATCAGCTGGGTGAGGCAGAGGCCCGGCCAGGGCCTGGAGTGGATCGGC |
| | | GAGATCTACCCCAGGAGCGGCAACACCTACCACAACGAGAAGTTCAAGG |
| | | ACAGGGTGACCATGACCGCCGACACCAGCACCAGCACCGTGTACATGGA |
| | | GCTGAGCAGCCTGAGGAGCGAGGACACCGCCGTGTACTACTGCGCCAGG |
| | | AGCATGATCACCACCAGGTACTGGTACTTCGACGTGTGGGGCAAGGGCA |
| | | CCACCGTGACCGTGAGCAGC |
| 100 | hz20-5 VL | DIVMTQSPDSLAVSLGERATINCRASESVDTYGNSFMHWYQQKPGQPPK |
| | | LLIYLASNLESGVPDRFSGSGSGTDFTLTISSLQAEDVAVYYCQQNNED |
| | | PLTFGAGTKLELK |
| 101 | hz20-5 VL DNA | GACATCGTGATGACCCAGTCTCCCGATTCCCTGGCCGTGAGCCTGGGAG |
| | sequence | AGAGGGCTACCATCAACTGTCGGGCTTCCGAGAGCGTGGACACATACGG |
| | | CAATTCTTTCATGCACTGGTATCAGCAGAAGCCCGGCCAGCCCCCTAAG |
| | | CTGCTGATCTACCTGGCTAGCAACCTGGAGTCTGGCGTGCCTGACAGAT |
| | | TCTCTGGCTCCGGCAGCGGCACCGACTTCACCCTGACAATCTCCAGCCT |
| | | GCAGGCCGAGGACGTGGCCGTGTACTATTGCCAGCAGAACAATGAGGAC |
| | | CCCCTGACCTTCGGCGCTGGCACAAAGCTGGAGCTGAAG |
| 102 | hz20-6 VH | QVQLVQSGAEVKKPGASVKLSCKATGYTFTNYGISWVRQAPGQGLEWIG |
| | | EIYPRSGNTYHNEKFKDRVTLTADKSSSTAYMEFRSLRSDDTAVYFCAR |
| | | SMITTRYWYFDVWGQGTLVTVSS |
| 103 | hz20-6 VH DNA | CAGGTGCAGCTGGTGCAGAGCGGCGCCGAGGTGAAGAAGCCCGGCGCCA |
| | sequence | GCGTGAAGCTGAGCTGCAAGGCCACCGGCTACACCTTCACCAACTACGG |
| | | CATCAGCTGGGTGAGGCAGGCCCCCGGCCAGGGCCTGGAGTGGATCGGC |
| | | GAGATCTACCCCAGGAGCGGCAACACCTACCACAACGAGAAGTTCAAGG |
| | | ACAGGGTGACCCTGACCGCCGACAAGAGCAGCAGCACCGCCTACATGGA |
| | | GTTCAGGAGCCTGAGGAGCGACGACACCGCCGTGTACTTCTGCGCCAGG |
| | | AGCATGATCACCACCAGGTACTGGTACTTCGACGTGTGGGGCCAGGGCA |
| | | CCCTGGTGACCGTGAGCAGC |
| 104 | hz20-6 VL | DIQMTQSPSSLSASVGDRVTITCRASESVDTYGNSFMHWYQQKPGKAPK |
| | | LLIYLASNLESGVPSRFSGSGSGTDFTLTISSLQPEDFATYYCQQNNED |
| | | PLTFGQGTKVEIK |
| 105 | hz20-6 VL DNA | GATATCCAGATGACCCAGTCCCCATCCAGCCTGTCTGCCTCCGTGGGCG |
| | sequence | ATAGGGTGACCATCACATGTCGGGCTAGCGAGTCTGTGGACACCTACGG |
| | | CAACAGCTTCATGCACTGGTATCAGCAGAAGCCAGGCAAGGCCCCCAAG |
| | | CTGCTGATCTACCTGGCTTCCAATCTGGAGAGCGGCGTGCCCTCTAGAT |
| | | TCTCCGGCAGCGGCTCTGGCACCGACTTTACCCTGACAATCTCTTCCCT |
| | | GCAGCCTGAGGATTTCGCCACATACTATTGCCAGCAGAACAATGAGGAC |
| | | CCACTGACCTTTGGCCAGGGCACAAAGGTGGAGATCAAG |
| 106 | hz20-7 VH | QVQLVQSGAEVKKPGASVKVSCKASGYTFTNYGISWVRQAPGQGLEWMG |
| | | EIYPRSGNTYHNEKFKDRVTMTTDTSTSTAYMELRSLRSDDTAVYYCAR |
| | | SMITTRYWYFDVWGKGTTVTVSS |
| 107 | hz20-7 VH DNA | CAGGTGCAGCTGGTGCAGTCTGGCGCCGAGGTGAAGAAGCCAGGCGCTT |
| | sequence | CCGTGAAGGTGAGCTGTAAGGCCTCTGGCTACACCTTCACAAACTATGG |
| | | AATCTCCTGGGTGAGGCAGGCTCCAGGACAGGGACTGGAGTGGATGGGC |
| | | GAGATCTACCCTAGAAGCGGCAACACATATCACAATGAGAAGTTTAAGG |
| | | ACCGCGTGACCATGACCACAGATACCTCTACATCCACCGCCTACATGGA |
| | | GCTGAGGAGCCTGCGGTCTGACGATACAGCCGTGTACTATTGCGCTAGA |
| | | TCCATGATCACCACACGCTACTGGTATTTCGACGTGTGGGGCAAGGGCA |
| | | CCACAGTGACCGTGTCCAGC |
| 108 | hz20-7 VL | DIVMTQSPDSLAVSLGERATINCRASESVDTYGNSFMHWYQQKPGQPPK |
| | | LLIYLASNLESGVPDRFSGSGSGTDFTLTISSLQAEDVAVYYCQQNNED |
| | | PLTFGQGTKLEIK |
| 109 | hz20-7 VL DNA | GACATCGTGATGACCCAGTCTCCCGATTCCCTGGCCGTGAGCCTGGGAG |
| | sequence | AGAGGGCTACCATCAACTGTCGGGCTTCCGAGAGCGTGGACACATACGG |
| | | CAATTCTTTCATGCACTGGTATCAGCAGAAGCCCGGCCAGCCCCCTAAG |
| | | CTGCTGATCTACCTGGCTAGCAACCTGGAGTCTGGCGTGCCTGACAGAT |
| | | TCTCTGGCTCCGGCAGCGGAACCGACTTCACCCTGACAATCTCCAGCCT |
| | | GCAGGCCGAGGACGTGGCCGTGTACTATTGCCAGCAGAACAATGAGGAC |
| | | CCCCTGACCTTCGGCCAGGGCACAAAGCTGGAGATCAAG |
| 110 | hz20-8 VH | QVQLVQSGAEVKKPGASVKVSCKASGYTFTNYGISWVRQAPGQGLEWMG |
| | | EIYPRSGNTYHNEKFKDRVTMTTDTSTSTAYMELRSLRSDDTAVYYCAR |
| | | SMITTRYWYFDVWGKGTTVTVSS |
| 111 | hz20-8 VH DNA | CAGGTGCAGCTGGTGCAGTCTGGCGCCGAGGTGAAGAAGCCAGGCGCTT |
| | sequence | CCGTGAAGGTGAGCTGTAAGGCCTCTGGCTACACCTTCACAAACTATGG |
| | | AATCTCCTGGGTGAGGCAGGCTCCAGGACAGGGACTGGAGTGGATGGGC |
| | | GAGATCTACCCTAGAAGCGGCAACACATATCACAATGAGAAGTTTAAGG |
| | | ACCGCGTGACCATGACCACAGATACCTCTACATCCACCGCCTACATGGA |
| | | GCTGAGGAGCCTGCGGTCTGACGATACAGCCGTGTACTATTGCGCTAGA |
| | | TCCATGATCACCACACGCTACTGGTATTTCGACGTGTGGGGCAAGGGCA |
| | | CCACAGTGACCGTGTCCAGC |
| 112 | hz20-8 VL | DIVMTQSPDSLAVSLGERATINCRASESVDTYGNSFMHWYQQKPGQPPK |
| | | LLIYLASNLESGVPDRFSGSGSGTDFTLTIDSLQAEDVAVYYCQQNNED |
| | | PLTFGQGTKLEIK |
| 113 | hz20-8 VL DNA | GATATCGTGATGACCCAGTCCCCCGACAGCCTGGCCGTGTCTCTGGGAG |
| | sequence | AGAGGGCCACCATCAACTGTCGGGCTAGCGAGTCTGTGGATACATACGG |
| | | CAATTCCTTCATGCACTGGTATCAGCAGAAGCCCGGCCAGCCCCCTAAG |
| | | CTGCTGATCTACCTGGCTTCTAACCTGGAGTCCGGCGTGCCTGACAGAT |
| | | TCTCCGGCAGCGGCTCTGGCACCGACTTTACCCTGACAATCGATTCCCT |
| | | GCAGGCCGAGGACGTGGCCGTGTACTATTGCCAGCAGAACAATGAGGAC |
| | | CCCCTGACCTTCGGCCAGGGCACAAAGCTGGAGATCAAG |
| 114 | hz20-9 VH | QVQLVQSGAEVKKPGASVKVSCKASGYTFTNYGISWVRQAPGQGLEWMG |
| | | EIYPRSGNTYHNEKFKDRVTMTTDKSTSTAYMELRSLRSDDTAVYYCAR |
| | | SMITTRYWYFDVWGKGTTVTVSS |
| 115 | hz20-9 VH DNA | CAGGTGCAGCTGGTGCAGTCTGGCGCCGAGGTGAAGAAGCCAGGCGCTT |
| | sequence | CCGTGAAGGTGAGCTGTAAGGCCTCTGGCTACACCTTCACAAACTATGG |
| | | AATCTCCTGGGTGAGGCAGGCTCCAGGACAGGGACTGGAGTGGATGGGC |
| | | GAGATCTACCCTAGAAGCGGCAACACCTATCACAATGAGAAGTTTAAGG |
| | | ACCGCGTGACAATGACCACCGATAAGTCTACCTCCACAGCCTACATGGA |
| | | GCTGAGGAGCCTGCGGTCTGACGATACCGCCGTGTACTATTGCGCTAGA |
| | | TCCATGATCACCACACGCTACTGGTATTTCGACGTGTGGGGCAAGGGCA |
| | | CCACAGTGACAGTGTCCAGC |
| 116 | hz20-9 VL | DIVMTQSPDSLAVSLGERATINCRASESVDTYGNSFMHWYQQKPGQPPK |
| | | LLIYLASNLESGVPDRFSGSGSGTDFTLTISSLQAEDVAVYYCQQNNED |
| | | PLTFGQGTKLEIK |
| 117 | hz20-9 VL DNA | GACATCGTGATGACCCAGTCTCCCGATTCCCTGGCCGTGAGCCTGGGAG |
| | sequence | AGAGGGCTACCATCAACTGTCGGGCTTCCGAGAGCGTGGACACATACGG |
| | | CAATTCTTTCATGCACTGGTATCAGCAGAAGCCCGGCCAGCCCCCTAAG |
| | | CTGCTGATCTACCTGGCTAGCAACCTGGAGTCTGGCGTGCCTGACAGAT |
| | | TCTCTGGCTCCGGCAGCGGAACCGACTTCACCCTGACAATCTCCAGCCT |
| | | GCAGGCCGAGGACGTGGCCGTGTACTATTGCCAGCAGAACAATGAGGAC |
| | | CCCCTGACCTTCGGCCAGGGCACAAAGCTGGAGATCAAG |
| 118 | hz20-10 VH | QVQLVQSGAEVKKPGASVKVSCKASGYTFTNYGISWVRQAPGQGLEWMG |
| | | EIYPRSGNTYHNEKFKDRVTMTTDKSTSTAYMELRSLRSDDTAVYYCAR |
| | | SMITTRYWYFDVWGKGTTVTVSS |
| 119 | hz20-10 VH DNA | CAGGTGCAGCTGGTGCAGTCTGGCGCCGAGGTGAAGAAGCCAGGCGCTT |
| | sequence | CCGTGAAGGTGAGCTGTAAGGCCTCTGGCTACACCTTCACAAACTATGG |
| | | AATCTCCTGGGTGAGGCAGGCTCCAGGACAGGGACTGGAGTGGATGGGC |
| | | GAGATCTACCCTAGAAGCGGCAACACCTATCACAATGAGAAGTTTAAGG |
| | | ACCGCGTGACAATGACCACCGATAAGTCTACCTCCACAGCCTACATGGA |
| | | GCTGAGGAGCCTGCGGTCTGACGATACCGCCGTGTACTATTGCGCTAGA |
| | | TCCATGATCACCACACGCTACTGGTATTTCGACGTGTGGGGCAAGGGCA |
| | | CCACAGTGACAGTGTCCAGC |
| 120 | hz20-10 VL | DIVMTQSPDSLAVSLGERATINCRASESVDTYGNSFMHWYQQKPGQPPK |
| | | LLIYLASNLESGVPDRFSGSGSGTDFTLTIDSLQAEDVAVYYCQQNNED |
| | | PLTFGQGTKLEIK |
| 121 | hz20-10 VL DNA | GATATCGTGATGACCCAGTCCCCCGACAGCCTGGCCGTGTCTCTGGGAG |
| | sequence | AGAGGGCCACCATCAACTGTCGGGCTAGCGAGTCTGTGGATACATACGG |
| | | CAATTCCTTCATGCACTGGTATCAGCAGAAGCCCGGCCAGCCCCCTAAG |
| | | CTGCTGATCTACCTGGCTTCTAACCTGGAGTCCGGCGTGCCTGACAGAT |
| | | TCTCCGGCAGCGGCTCTGGCACCGACTTTACCCTGACAATCGATTCCCT |
| | | GCAGGCCGAGGACGTGGCCGTGTACTATTGCCAGCAGAACAATGAGGAC |
| | | CCCCTGACCTTCGGCCAGGGCACAAAGCTGGAGATCAAG |
| 122 | hz20-11 VH | QVQLVQSGAEVAKPGASVKVSCKASGYTFTNYGISWVRQRPGQGLEWMG |
| | | EIYPRSGNTYHNEKFKDRVTMTTDTSTSTAYMELRSLRSEDTAVYYCAR |
| | | SMITTRYWYFDVWGKGTTVTVSS |
| 123 | hz20-11 VH DNA | CAGGTGCAGCTGGTGCAGTCTGGAGCTGAGGTGGCTAAGCCAGGAGCTT |
| | sequence | CCGTGAAGGTGAGCTGTAAGGCTTCTGGCTACACCTTCACAAACTATGG |
| | | CATCTCCTGGGTGAGGCAGAGGCCAGGACAGGGACTGGAGTGGATGGGC |
| | | GAGATCTACCCTAGGAGCGGCAACACATATCACAATGAGAAGTTTAAGG |
| | | ACAGGGTGACCATGACCACAGATACCTCTACATCCACCGCCTACATGGA |
| | | GCTGAGAAGCCTGCGCTCTGAGGACACAGCCGTGTACTATTGCGCTAGG |
| | | TCCATGATCACCACACGGTACTGGTATTTCGACGTGTGGGGCAAGGGCA |
| | | CCACAGTGACCGTGTCCAGC |
| 124 | hz20-11 VL | DIVMTQSPDSLAVSLGERATINCRASESVDTYGNSFMHWYQQKPGQPPK |
| | | LLIYLASNLESGVPDRFSGSGSGTDFTLTISSLQAEDVAVYYCQQNNED |
| | | PLTFGQGTKLEIK |
| 125 | hz20-11 VLDNA | GACATCGTGATGACCCAGTCTCCCGATTCCCTGGCCGTGAGCCTGGGAG |
| | sequence | AGAGGGCTACCATCAACTGTCGGGCTTCCGAGAGCGTGGACACATACGG |
| | | CAATTCTTTCATGCACTGGTATCAGCAGAAGCCCGGCCAGCCCCCTAAG |
| | | CTGCTGATCTACCTGGCTAGCAACCTGGAGTCTGGCGTGCCTGACAGAT |
| | | TCTCTGGCTCCGGCAGCGGAACCGACTTCACCCTGACAATCTCCAGCCT |
| | | GCAGGCCGAGGACGTGGCCGTGTACTATTGCCAGCAGAACAATGAGGAC |
| | | CCCCTGACCTTCGGCCAGGGCACAAAGCTGGAGATCAAG |
| 126 | hz20-12 VH | QVQLVQSGAEVAKPGASVKVSCKASGYTFTNYGISWVRQRPGQGLEWMG |
| | | EIYPRSGNTYHNEKFKDRVTMTTDTSTSTAYMELRSLRSEDTAVYYCAR |
| | | SMITTRYWYFDVWGKGTTVTVSS |
| 127 | hz20-12 VH DNA | CAGGTGCAGCTGGTGCAGTCTGGAGCTGAGGTGGCTAAGCCAGGAGCTT |
| | sequence | CCGTGAAGGTGAGCTGTAAGGCTTCTGGCTACACCTTCACAAACTATGG |
| | | CATCTCCTGGGTGAGGCAGAGGCCAGGACAGGGACTGGAGTGGATGGGC |
| | | GAGATCTACCCTAGGAGCGGCAACACATATCACAATGAGAAGTTTAAGG |
| | | ACAGGGTGACCATGACCACAGATACCTCTACATCCACCGCCTACATGGA |
| | | GCTGAGAAGCCTGCGCTCTGAGGACACAGCCGTGTACTATTGCGCTAGG |
| | | TCCATGATCACCACACGGTACTGGTATTTCGACGTGTGGGGCAAGGGCA |
| | | CCACAGTGACCGTGTCCAGC |
| 128 | hz20-12 VL | DIVMTQSPDSLAVSLGERATINCRASESVDTYGNSFMHWYQQKPGQPPK |
| | | LLIYLASNLESGVPDRFSGSGSGTDFTLTIDSLQAEDVAVYYCQQNNED |
| | | PLTFGQGTKLEIK |
| 129 | hz20-12 VL DNA | GATATCGTGATGACCCAGTCCCCCGACAGCCTGGCCGTGTCTCTGGGAG |
| | sequence | AGAGGGCCACCATCAACTGTCGGGCTAGCGAGTCTGTGGATACATACGG |
| | | CAATTCCTTCATGCACTGGTATCAGCAGAAGCCCGGCCAGCCCCCTAAG |
| | | CTGCTGATCTACCTGGCTTCTAACCTGGAGTCCGGCGTGCCTGACAGAT |
| | | TCTCCGGCAGCGGCTCTGGCACCGACTTTACCCTGACAATCGATTCCCT |
| | | GCAGGCCGAGGACGTGGCCGTGTACTATTGCCAGCAGAACAATGAGGAC |
| | | CCCCTGACCTTCGGCCAGGGCACAAAGCTGGAGATCAAG |
| 130 | hz20-13 VH | QVQLVQSGAEVKKPGASVKVSCKASGYTFTNYGISWVRQRPGQGLEWMG |
| | | EIYPRSGNTYHNEKFKDRVTMTTDKSTSTAYMELRSLRSEDTAVYYCAR |
| | | SMITTRYWYFDVWGKGTTVTVSS |
| 131 | hz20-13 VH DNA | CAGGTGCAGCTGGTGCAGTCTGGCGCCGAGGTGAAGAAGCCAGGCGCTT |
| | sequence | CCGTGAAGGTGAGCTGTAAGGCCTCTGGCTACACCTTCACAAACTATGG |
| | | CATCTCCTGGGTGAGGCAGAGGCCAGGACAGGGACTGGAGTGGATGGGC |
| | | GAGATCTACCCTAGGAGCGGCAACACCTATCACAATGAGAAGTTTAAGG |
| | | ACAGGGTGACAATGACCACCGATAAGTCTACCTCCACAGCTTACATGGA |
| | | GCTGAGAAGCCTGCGCTCTGAGGACACCGCCGTGTACTATTGCGCTAGG |
| | | TCCATGATCACCACACGGTACTGGTATTTCGACGTGTGGGGCAAGGGCA |
| | | CCACAGTGACAGTGTCCAGC |
| 132 | hz20-13 VL | DIVMTQSPDSLAVSLGERATINCRASESVDTYGNSFMHWYQQKPGQPPK |
| | | LLIYLASNLESGVPDRFSGSGSGTDFTLTISSLQAEDVAVYYCQQNNED |
| | | PLTFGQGTKLEIK |
| 133 | hz20-13 VL DNA | GACATCGTGATGACCCAGTCTCCCGATTCCCTGGCCGTGAGCCTGGGAG |
| | sequence | AGAGGGCTACCATCAACTGTCGGGCTTCCGAGAGCGTGGACACATACGG |
| | | CAATTCTTTCATGCACTGGTATCAGCAGAAGCCCGGCCAGCCCCCTAAG |
| | | CTGCTGATCTACCTGGCTAGCAACCTGGAGTCTGGCGTGCCTGACAGAT |
| | | TCTCTGGCTCCGGCAGCGGAACCGACTTCACCCTGACAATCTCCAGCCT |
| | | GCAGGCCGAGGACGTGGCCGTGTACTATTGCCAGCAGAACAATGAGGAC |
| | | CCCCTGACCTTCGGCCAGGGCACAAAGCTGGAGATCAAG |
| 134 | hz20-14 VH | QVQLVQSGAEVKKPGASVKVSCKASGYTFTNYGISWVRQRPGQGLEWMG |
| | | EIYPRSGNTYHNEKFKDRVTMTTDKSTSTAYMELRSLRSEDTAVYYCAR |
| | | SMITTRYWYFDVWGKGTTVTVSS |
| 135 | hz20-14 VH DNA | CAGGTGCAGCTGGTGCAGTCTGGCGCCGAGGTGAAGAAGCCAGGCGCTT |
| | sequence | CCGTGAAGGTGAGCTGTAAGGCCTCTGGCTACACCTTCACAAACTATGG |
| | | CATCTCCTGGGTGAGGCAGAGGCCAGGACAGGGACTGGAGTGGATGGGC |
| | | GAGATCTACCCTAGGAGCGGCAACACCTATCACAATGAGAAGTTTAAGG |
| | | ACAGGGTGACAATGACCACCGATAAGTCTACCTCCACAGCTTACATGGA |
| | | GCTGAGAAGCCTGCGCTCTGAGGACACCGCCGTGTACTATTGCGCTAGG |
| | | TCCATGATCACCACACGGTACTGGTATTTCGACGTGTGGGGCAAGGGCA |
| | | CCACAGTGACAGTGTCCAGC |
| 136 | hz20-14 VL | DIVMTQSPDSLAVSLGERATINCRASESVDTYGNSFMHWYQQKPGQPPK |
| | | LLIYLASNLESGVPDRFSGSGSGTDFTLTIDSLQAEDVAVYYCQQNNED |
| | | PLTFGQGTKLEIK |
| 137 | hz20-14 VL DNA | GATATCGTGATGACCCAGTCCCCCGACAGCCTGGCCGTGTCTCTGGGAG |
| | sequence | AGAGGGCCACCATCAACTGTCGGGCTAGCGAGTCTGTGGATACATACGG |
| | | CAATTCCTTCATGCACTGGTATCAGCAGAAGCCCGGCCAGCCCCCTAAG |
| | | CTGCTGATCTACCTGGCTTCTAACCTGGAGTCCGGCGTGCCTGACAGAT |
| | | TCTCCGGCAGCGGCTCTGGCACCGACTTTACCCTGACAATCGATTCCCT |
| | | GCAGGCCGAGGACGTGGCCGTGTACTATTGCCAGCAGAACAATGAGGAC |
| | | CCCCTGACCTTCGGCCAGGGCACAAAGCTGGAGATCAAG |
| 138 | hz13-5 VH_P52aK | EVLLVQSGAEVKKPGASVKLSCTSSDFNIKDHFIHWVRQAPGKGLEWMG |
| | | RIDKEDDETKYAPKFQDRVTMTADTSTDTAYMELSSLRSEDTAVFYCAG |
| | | YGNYEGAMDYWGQGTLVTVSS |
| 139 | hz13-5 VH_P52aK | GAGGTGCTGCTGGTGCAGTCTGGCGCTGAAGTGAAGAAGCCCGGAGCTT |
| | DNA sequence | CTGTGAAGCTGTCCTGCACATCCTCCGACTTCAACATCAAGGACCACTT |
| | | CATCCACTGGGTGCGGCAAGCTCCTGGCAAGGGCCTGGAATGGATGGGC |
| | | CGGATCGACAAAGAGGACGACGAGACCAAGTACGCCCCTAAGTTCCAGG |
| | | ACAGAGTGACCATGACCGCCGATACCTCTACCGATACAGCCTACATGGA |
| | | ACTGTCTAGCCTGAGATCCGAGGATACCGCTGTGTTTTATTGTGCCGGA |
| | | TACGGCAACTACGAGGGCGCCATGGACTACTGGGGCCAGGGCACCCTGG |
| | | TCACCGTGTCCAGC |
| 140 | hz13-5 VH_134H P52aK | EVLLVQSGAEVKKPGASVKLSCTSSDFNIKDHFHHWVRQAPGKGLEWMG |
| | | RIDKEDDETKYAPKFQDRVTMTADTSTDTAYMELSSLRSEDTAVFYCAG |
| | | YGNYEGAMDYWGQGTLVTVSS |
| 141 | hz13-5 VH_134H P52aK | GAGGTGCTGCTGGTGCAGTCTGGCGCTGAAGTGAAGAAGCCCGGAGCTT |
| | DNA sequence | CCGTGAAGCTGTCTTGTACCTCCTCCGACTTCAACATCAAGGATCATTT |
| | | TCACCACTGGGTGCGGCAAGCTCCTGGCAAGGGCCTGGAATGGATGGGA |
| | | CGGATCGACAAAGAGGACGACGAGACAAAGTACGCCCCTAAGTTCCAGG |
| | | ACAGAGTGACCATGACCGCTGATACCTCTACCGACACCGCCTACATGGA |
| | | ACTGAGCTCTCTGAGATCCGAGGATACAGCCGTGTTCTACTGCGCCGGC |
| | | TATGGCAACTACGAGGGCGCCATGGACTACTGGGGCCAGGGCACCCTGG |
| | | TCACCGTGTCCAGC |
| 142 | hz13-5 VH_134H H35K | EVLLVQSGAEVKKPGASVKLSCTSSDFNIKDHFHKWVRQAPGKGLEWMG |
| | P52aK | RIKPEDDETKYAPKFQDRVTMTADTSTDTAYMELSSLRSEDTAVFYCAG |
| | | YGNYEGAMDYWGQGTLVTVSS |
| 143 | hz13-5 VH_134H H35K | GAGGTGCTGCTGGTGCAGTCTGGCGCTGAAGTGAAAAAACCCGGAGCTA |
| | P52aK DNA sequence | GCGTGAAGCTGTCTTGTACCTCCTCCGACTTCAACATCAAGGACCACTT |
| | | TCACAAGTGGGTGCGGCAAGCTCCTGGCAAGGGCCTGGAATGGATGGGC |
| | | CGGATCAAGCCTGAGGACGACGAGACAAAGTACGCCCCTAAGTTCCAGG |
| | | ACAGAGTGACCATGACCGCTGATACCTCTACCGATACCGCCTACATGGA |
| | | ACTGTCCAGCCTGAGATCCGAGGATACAGCCGTGTTCTACTGCGCCGGA |
| | | TATGGCAACTACGAGGGCGCCATGGACTACTGGGGCCAGGGCACCCTGG |
| | | TCACCGTGTCCTCT |
| 144 | hz13-5 VH_134H H35K | EVLLVQSGAEVKKPGASVKLSCTSSDFNIKDHFHKWVRQAPGKGLEWMG |
| | | RIDPEDDETKYAPKFQDRVTMTADTSTDTAYMELSSLRSEDTAVFYCAG |
| | | YGNYEGAMDYWGQGTLVTVSS |
| 145 | hz13-5 VH_134H H35K | GAGGTGCTGCTGGTGCAGTCCGGCGCTGAAGTGAAAAAGCCTGGAGCCT |
| | DNA sequence | CCGTGAAGCTGTCCTGCACCAGCAGCGACTTCAACATCAAGGACCACTT |
| | | TCACAAGTGGGTGCGGCAAGCTCCTGGCAAGGGCCTGGAATGGATGGGC |
| | | AGAATCGACCCTGAGGACGACGAGACCAAGTACGCCCCCAAGTTCCAGG |
| | | ACAGAGTGACCATGACCGCTGATACCTCTACAGATACCGCCTACATGGA |
| | | ACTCTCTTCTCTGAGATCCGAGGATACCGCCGTGTTCTATTGTGCCGGC |
| | | TACGGCAACTACGAGGGCGCCATGGACTACTGGGGCCAGGGCACCCTGG |
| | | TCACAGTGTCCTCT |
| 146 | hz13-5 VH_134H | EVLLVQSGAEVKKPGASVKLSCTSSDFNIKDHFHHWVRQAPGKGLEWMG |
| | | RIDPEDDETKYAPKFQDRVTMTADTSTDTAYMELSSLRSEDTAVFYCAG |
| | | YGNYEGAMDYWGQGTLVTVSS |
| 147 | hz13-5 VH 134H DNA | GAAGTGCTGCTGGTGCAGAGCGGAGCTGAGGTGAAAAAGCCCGGAGCTT |
| | sequence | CTGTGAAGCTGTCTTGTACCTCCTCCGACTTCAACATCAAGGACCACTT |
| | | TCATCACTGGGTGCGGCAAGCTCCTGGCAAGGGCCTGGAATGGATGGGC |
| | | CGGATCGATCCTGAGGACGACGAGACAAAGTACGCCCCTAAGTTCCAGG |
| | | ACAGAGTGACCATGACCGCTGATACCTCTACCGACACCGCCTACATGGA |
| | | ACTGTCCTCTCTGAGATCCGAGGATACAGCCGTGTTCTACTGCGCCGGC |
| | | TATGGCAACTACGAGGGCGCCATGGACTACTGGGGCCAGGGCACCCTGG |
| | | TCACCGTGTCCAGC |
| 148 | hz13-5 VH H35K | EVLLVQSGAEVKKPGASVKLSCTSSDFNIKDHFIKWVRQAPGKGLEWMG |
| | P52aK | RIDKEDDETKYAPKFQDRVTMTADTSTDTAYMELSSLRSEDTAVFYCAG |
| | | YGNYEGAMDYWGQGTLVTVSS |
| 149 | hz13-5 VH H35K | GAAGTGCTGCTGGTGCAGTCTGGCGCTGAGGTGAAAAAGCCCGGAGCTT |
| | P52aK DNA sequence | CCGTGAAGCTGTCCTGCACCTCTTCCGACTTCAACATCAAGGACCACTT |
| | | CATCAAGTGGGTGCGGCAAGCTCCTGGCAAGGGCCTGGAATGGATGGGC |
| | | CGGATCGACAAAGAGGACGACGAGACCAAGTACGCCCCTAAGTTCCAGG |
| | | ACAGAGTGACCATGACCGCTGATACAAGCACCGATACCGCCTACATGGA |
| | | ACTGTCTTCTCTGAGATCCGAGGATACAGCCGTGTTTTATTGTGCCGGC |
| | | TACGGCAACTACGAGGGCGCCATGGACTACTGGGGCCAGGGCACCCTGG |
| | | TCACCGTGTCCAGC |
| 150 | hz13-5 VH_H35K | EVLLVQSGAEVKKPGASVKLSCTSSDFNIKDHFIKWVRQAPGKGLEWMG |
| | | RIDPEDDETKYAPKFQDRVTMTADTSTDTAYMELSSLRSEDTAVFYCAG |
| | | YGNYEGAMDYWGQGTLVTVSS |
| 151 | hz13-5 VH H35K DNA | GAAGTGCTGCTGGTGCAGTCTGGCGCTGAGGTGAAAAAGCCTGGAGCCT |
| | sequence | CCGTGAAGCTGTCTTGTACCTCCTCCGACTTCAACATCAAGGACCACTT |
| | | CATCAAGTGGGTGCGGCAAGCTCCTGGCAAGGGCCTGGAATGGATGGGC |
| | | CGGATCGACCCCGAGGACGACGAGACCAAGTACGCCCCTAAGTTTCAGG |
| | | ACAGAGTGACCATGACCGCTGATACATCTACCGATACCGCCTACATGGA |
| | | ACTGAGCAGCCTGAGATCCGAGGATACAGCCGTGTTCTACTGCGCTGGC |
| | | TATGGCAACTACGAGGGCGCCATGGACTACTGGGGCCAGGGCACCCTGG |
| | | TCACCGTGTCCTCT |
| 152 | hz13-5 VH_H32W | EVLLVQSGAEVKKPGASVKLSCTSSDFNIKDWFIHWVRQAPGKGLEWMG |
| | | RIDPEDDETKYAPKFQDRVTMTADTSTDTAYMELSSLRSEDTAVFYCAG |
| | | YGNYEGAMDYWGQGTLVTVSS |
| 153 | hz13-5 VH_H32W | GAAGTGCTGCTGGTGCAGTCTGGCGCTGAGGTGAAAAAGCCTGGAGCCT |
| | DNA sequence | CCGTGAAGCTGTCCTGCACCTCCTCCGACTTCAACATCAAGGACTGGTT |
| | | CATCCACTGGGTCAGACAGGCTCCTGGCAAGGGCCTGGAATGGATGGGC |
| | | CGGATCGACCCCGAGGACGACGAGACCAAGTACGCCCCTAAGTTCCAGG |
| | | ACAGAGTGACCATGACCGCTGATACATCTACCGATACCGCCTACATGGA |
| | | ACTGTCTTCTCTGCGGAGCGAGGATACAGCCGTGTTTTATTGTGCTGGC |
| | | TACGGCAACTACGAGGGCGCCATGGACTACTGGGGCCAAGGAACCCTCG |
| | | TGACCGTGTCCAGC |
| 154 | hz13-5 VH_D31H | EVLLVQSGAEVKKPGASVKLSCTSSDFNIKHHFIHWVRQAPGKGLEWMG |
| | P52aK | RIDKEDDETKYAPKFQDRVTMTADTSTDTAYMELSSLRSEDTAVFYCAG |
| | | YGNYEGAMDYWGQGTLVTVSS |
| 155 | hz13-5 VH D31H | GAGGTGCTGCTGGTGCAGAGCGGAGCTGAAGTGAAGAAGCCTGGCGCTT |
| | P52aK DNA sequence | CTGTGAAGCTGTCCTGCACCTCCTCCGATTTCAACATCAAGCACCATTT |
| | | TATCCACTGGGTGCGGCAAGCTCCCGGCAAGGGCCTGGAATGGATGGGC |
| | | CGGATCGACAAAGAGGACGACGAGACCAAGTACGCCCCTAAGTTCCAGG |
| | | ACAGAGTGACCATGACCGCTGATACATCTACCGACACCGCCTACATGGA |
| | | ACTGTCTAGCCTGAGATCCGAGGATACAGCCGTGTTCTACTGTGCCGGC |
| | | TATGGAAACTACGAGGGCGCCATGGACTACTGGGGCCAGGGCACCCTGG |
| | | TCACCGTGTCCTCT |
| 156 | hz13-5 VH_D31H 134H | EVLLVQSGAEVKKPGASVKLSCTSSDFNIKHHFHHWVRQAPGKGLEWMG |
| | P52aK | RIDKEDDETKYAPKFQDRVTMTADTSTDTAYMELSSLRSEDTAVFYCAG |
| | | YGNYEGAMDYWGQGTLVTVSS |
| 157 | hz13-5 VH_D31H 134H | GAAGTGCTGCTGGTGCAGTCTGGCGCCGAGGTGAAGAAGCCCGGAGCTT |
| | P52aK DNA sequence | CTGTGAAGCTGTCCTGCACCTCCTCCGACTTCAACATCAAGCACCATTT |
| | | TCACCACTGGGTGCGGCAAGCTCCTGGCAAGGGCCTGGAATGGATGGGC |
| | | CGGATCGATAAAGAGGACGACGAGACAAAGTACGCCCCTAAGTTCCAGG |
| | | ACAGAGTGACCATGACCGCTGATACCTCTACCGACACCGCCTACATGGA |
| | | ACTGTCTAGCCTGAGATCCGAGGATACAGCCGTGTTCTATTGTGCCGGC |
| | | TACGGCAACTACGAGGGAGCCATGGACTACTGGGGCCAGGGCACCCTGG |
| | | TCACCGTGTCCAGC |
| 158 | hz13-5 VH_D31H I34H | EVLLVQSGAEVKKPGASVKLSCTSSDFNIKHHFHKWVRQAPGKGLEWMG |
| | H35K P52aK | RIDKEDDETKYAPKFQDRVTMTADTSTDTAYMELSSLRSEDTAVFYCAG |
| | | YGNYEGAMDYWGQGTLVTVSS |
| 159 | hz13-5 VH D31H 134H | GAGGTGCTGCTGGTGCAGTCCGGCGCTGAAGTGAAAAAGCCTGGAGCTT |
| | H35K P52aK DNA | CTGTGAAGCTGTCCTGCACCTCTTCCGACTTCAACATCAAGCACCATTT |
| | sequence | CCACAAGTGGGTGCGGCAAGCCCCTGGCAAGGGCCTGGAATGGATGGGC |
| | | CGGATCGACAAAGAGGACGACGAGACCAAGTACGCCCCCAAGTTCCAGG |
| | | ACAGAGTGACCATGACCGCTGATACAAGCACCGATACCGCCTACATGGA |
| | | ACTGTCTTCTCTGAGATCCGAGGATACAGCCGTGTTTTATTGTGCTGGC |
| | | TACGGCAACTACGAGGGCGCCATGGACTACTGGGGCCAGGGCACCCTGG |
| | | TCACCGTGTCCAGC |
| 160 | hz13-5 VH_D31H 134H | EVLLVQSGAEVKKPGASVKLSCTSSDFNIKHHFHKWVRQAPGKGLEWMG |
| | H35K | RIDPEDDETKYAPKFQDRVTMTADTSTDTAYMELSSLRSEDTAVFYCAG |
| | | YGNYEGAMDYWGQGTLVTVSS |
| 161 | hz13-5 VH_D31H 134H | GAGGTGCTGCTGGTGCAGTCTGGAGCTGAAGTGAAAAAGCCTGGAGCTT |
| | H35K DNA sequence | CCGTGAAGCTGTCCTGCACCTCTAGCGACTTCAACATCAAGCACCATTT |
| | | TCACAAGTGGGTGCGGCAAGCTCCTGGCAAGGGCCTGGAATGGATGGGC |
| | | CGGATCGACCCCGAGGACGACGAGACAAAGTACGCCCCTAAGTTCCAGG |
| | | ACAGAGTGACCATGACCGCTGATACCTCCACCGACACCGCCTACATGGA |
| | | ACTCTCTTCTCTGAGATCCGAGGATACAGCCGTGTTCTATTGTGCCGGC |
| | | TACGGCAACTACGAGGGCGCCATGGATTACTGGGGCCAGGGCACCCTGG |
| | | TCACCGTGTCCAGC |
| 162 | hz13-5 VH_D31H 134H | EVLLVQSGAEVKKPGASVKLSCTSSDFNIKHHFHHWVRQAPGKGLEWMG |
| | | RIDPEDDETKYAPKFQDRVTMTADTSTDTAYMELSSLRSEDTAVFYCAG |
| | | YGNYEGAMDYWGQGTLVTVSS |
| 163 | hz13-5 VH_D31H 134H | GAGGTGCTGCTGGTGCAGTCCGGCGCTGAAGTGAAGAAACCTGGCGCTT |
| | DNA sequence | CTGTCAAGCTGTCCTGTACATCTAGCGACTTCAACATCAAGCACCACTT |
| | | CCATCACTGGGTGCGGCAAGCTCCTGGAAAGGGCCTGGAATGGATGGGC |
| | | AGAATCGACCCTGAGGACGACGAGACCAAGTACGCCCCCAAGTTTCAGG |
| | | ACAGAGTGACCATGACCGCCGATACCTCCACCGATACAGCCTACATGGA |
| | | ACTGTCTTCTCTGCGGTCCGAGGATACCGCTGTGTTCTACTGCGCCGGC |
| | | TATGGCAACTACGAGGGAGCCATGGACTACTGGGGCCAGGGCACCCTCG |
| | | TGACCGTGTCCAGC |
| 164 | hz13-5 VH_D31H | EVLLVQSGAEVKKPGASVKLSCTSSDFNIKHHFIKWVRQAPGKGLEWMG |
| | H35K P52aK | RIDKEDDETKYAPKFQDRVTMTADTSTDTAYMELSSLRSEDTAVFYCAG |
| | | YGNYEGAMDYWGQGTLVTVSS |
| 165 | hz13-5 VH_D31H | GAGGTGCTGCTGGTGCAGAGCGGAGCTGAAGTGAAAAAGCCCGGAGCTT |
| | H35K P52aK DNA | CTGTGAAGCTGTCTTGTACCTCCTCCGACTTCAACATCAAGCACCACTT |
| | sequence | CATCAAGTGGGTGCGGCAAGCTCCTGGCAAGGGCCTGGAATGGATGGGC |
| | | CGGATCGACAAAGAGGACGACGAGACCAAGTACGCCCCTAAGTTTCAGG |
| | | ACAGAGTGACCATGACCGCTGATACATCTACCGATACAGCCTACATGGA |
| | | ACTGTCCTCTCTGAGATCCGAGGATACCGCCGTGTTCTACTGCGCCGGC |
| | | TATGGCAACTACGAGGGCGCCATGGACTACTGGGGCCAGGGCACCCTGG |
| | | TCACCGTGTCCAGC |
| 166 | hz13-5 VH_D31H | EVLLVQSGAEVKKPGASVKLSCTSSDFNIKHHFIKWVRQAPGKGLEWMG |
| | H35K | RIDPEDDETKYAPKFQDRVTMTADTSTDTAYMELSSLRSEDTAVFYCAG |
| | | YGNYEGAMDYWGQGTLVTVSS |
| 167 | hz13-5 VH_D31H | GAGGTGCTGCTGGTGCAGTCTGGCGCTGAAGTGAAAAAGCCTGGAGCTT |
| | H35K DNA sequence | CCGTGAAGCTGTCCTGCACCTCTAGCGACTTCAACATCAAGCACCACTT |
| | | CATCAAGTGGGTGCGGCAAGCTCCTGGCAAGGGCCTGGAATGGATGGGC |
| | | CGGATCGATCCCGAGGACGACGAGACCAAGTACGCCCCTAAGTTCCAGG |
| | | ACAGAGTGACCATGACCGCTGATACCTCCACCGACACAGCCTACATGGA |
| | | ACTGTCTTCTCTGAGATCCGAGGATACCGCCGTGTTTTATTGTGCCGGC |
| | | TACGGCAACTACGAGGGAGCCATGGACTACTGGGGCCAGGGCACCCTGG |
| | | TCACAGTGTCCAGC |
| 168 | hz13-5 VH_D31H | EVLLVQSGAEVKKPGASVKLSCTSSDFNIKHHFIHWVRQAPGKGLEWMG |
| | | RIDPEDDETKYAPKFQDRVTMTADTSTDTAYMELSSLRSEDTAVFYCAG |
| | | YGNYEGAMDYWGQGTLVTVSS |
| 169 | hz13-5 VH_D31H DNA | GAGGTGCTGCTGGTGCAGTCTGGCGCTGAAGTGAAAAAGCCTGGAGCCT |
| | sequence | CCGTGAAGCTGTCTTGCACCTCCTCCGACTTCAACATCAAGCACCATTT |
| | | TATCCACTGGGTGCGGCAAGCTCCTGGCAAGGGCCTGGAATGGATGGGC |
| | | CGGATCGACCCCGAGGACGACGAGACCAAGTACGCCCCTAAGTTCCAGG |
| | | ACAGAGTGACCATGACCGCTGATACATCTACCGATACCGCCTACATGGA |
| | | ACTCAGCTCTCTGAGATCCGAGGATACAGCCGTGTTCTATTGTGCTGGC |
| | | TACGGCAACTACGAGGGCGCCATGGACTACTGGGGCCAGGGCACCCTGG |
| | | TCACCGTGTCCAGC |
| 170 | VL sequence for hz13-5 | DIQMTQSPSSLSASVGDRVTITCRASENVNNYGIGFMNWFQQKPGKAPK |
| | VH_P52aK, hz13-5 | LLIYAASRRGSGVPSRFSGSGSGTDFTLTISSLQPEDFATYFCQQSKEV |
| | VH_134H P52aK, hz13- | PYTFGQGTKVEIK |
| | 5 VH_134H H35K | |
| | P52aK, hz13-5 VH | |
| | 134H H35K, hz13-5 VH | |
| | 134H, hz13-5 VH_H35K | |
| | P52aK, hz13-5 VH | |
| | H35K, hz13-5 VH | |
| | H32W, hz13-5 VH | |
| | D31H P52aK, hz13-5 | |
| | VH_D31H 134H P52aK, | |
| | hz13-5 VH_D31H 134H | |
| 171 | H35K P52aK, hz13-5 | GACATCCAGATGACCCAGTCTCCATCCAGCCTGTCTGCCTCCGTGGGCG |
| | VH_D31H 134H H35K, | ATAGAGTGACCATCACATGTCGCGCTTCCGAGAACGTGAACAATTACGG |
| | hz13-5 VH_D31H 134H, | CATCGGCTTCATGAATTGGTTTCAGCAGAAGCCAGGCAAGGCCCCCAAG |
| | hz13-5 VH_D31H | CTGCTGATCTATGCTGCTAGCAGGAGGGGCTCTGGAGTGCCCTCCAGGT |
| | H35K P52aK, hz13-5 | TCAGCGGCTCTGGCTCCGGAACCGACTTTACCCTGACAATCTCTTCCCT |
| | VH_D31H H35K, hz13- | GCAGCCTGAGGATTTCGCTACATACTTTTGCCAGCAGTCCAAGGAGGTG |
| | 5 VH_D31H | CCATATACCTTCGGCCAGGGCACAAAGGTGGAGATCAAG |
| 172 | VL DNA sequence for | DIQMTQSPSSLSASVGDRVTITCRASENVNNYGHGFMNWFQQKPGKAPK |
| | hz13-5 VH_P52aK, | LLIYAASRRGSGVPSRFSGSGSGTDFTLTISSLQPEDFATYFCQQSKEV |
| | hz13-5 VH_134H | PYTFGQGTKVEIK |
| | P52aK, hz13-5 VH | |
| | I34H H35K P52aK, | |
| | hz13-5 VH_134H H35K, | |
| | hz13-5 VH_134H, hz13- | |
| | 5 VH_H35K P52aK, | |
| | hz13-5 VH_H35K, | |
| | hz13-5 VH_H32W, | |
| | hz13-5 VH_D31H | |
| | P52aK, hz13-5 VH | |
| | D31H 134H P52aK, | |
| | hz13-5 VH_D31H 134H | |
| | H35K P52aK, hz13-5 | |
| | VH_D31H 134H H35K, | |
| | hz13-5 VH_D31H 134H, | |
| | hz13-5 VH_D31H | |
| | H35K P52aK, hz13-5 | |
| | VH_D31H H35K, hz13- | |
| | 5 VH_D31H | |
| | VL_130H protein | |
| | sequence for hz13-5 | |
| | VH_P52aK::Vk_I30H, | |
| | hz13-5 VH_134H, | |
| | P52aK::Vk_I30H, hz13- | |
| | 5 VH_134H H35K | |
| | P52aK::Vk_I30H, hz13- | |
| | 5 VH_134H | |
| | H35K::VK I30H, hz13-5 | |
| | VH_134H::Vk_I30H, | |
| | hz13-5 VH_H35K | |
| | P52aK::Vk_I30H, hz13- | |
| | 5 VH_H35K::Vk_I30H, | |
| | hz13-5 VH_D31H | |
| | P52aK::Vk_I30H, hz13- | |
| | 5 VH_D31H 134H | |
| | P52aK: Vk_I30H, hz13- | |
| | 5 VH_D31H 134H | |
| | H35K P52aK::Vk_I30H, | |
| | hz13-5 VH_D31H 134H | |
| | H35K::Vk_130H, hz13- | |
| | 5 VH_D31H | |
| | 134H::Vk_I30H, hz13-5 | |
| | VH_D31H H35K | |
| | P52aK::Vk_I30H, hz13- | |
| | 5 VH_D31H | |
| | H35K::Vk_I30H, hz13-5 | |
| | VH_D31H::Vk_I30H | |
| 173 | Vk_I30H DNA sequence | GATATCCAGATGACCCAGTCTCCATCCAGCCTGTCTGCTTCCGTGGGCG |
| | for hz13-5 VH | ACAGAGTCACAATCACCTGTAGAGCCTCCGAAAACGTGAACAACTACGG |
| | P52aK::Vk_I30H, hz13- | CCATGGCTTCATGAACTGGTTCCAGCAGAAGCCTGGCAAGGCCCCCAAA |
| | 5 VH_134H, | CTGCTGATCTACGCTGCTTCACGCCGGGGATCTGGCGTGCCCTCCCGGT |
| | P52aK::Vk_I30H, hz13- | TCTCCGGCTCCGGCTCTGGAACCGATTTTACCCTGACCATCTCCTCCCT |
| | 5 VH_134H H35K | GCAGCCTGAGGACTTCGCCACCTACTTCTGCCAGCAGTCCAAAGAGGTG |
| | P52aK::Vk_I30H, hz13- | CCTTACACCTTTGGCCAGGGCACCAAAGTGGAAATCAAG |
| | 5 VH_134H | |
| | H35K::VK I30H, hz13-5 | |
| | VH_134H::Vk_I30H, | |
| | hz13-5 VH_H35K | |
| | P52aK::Vk_I30H, hz13- | |
| | 5 VH_H35K::Vk_I30H, | |
| | hz13-5 VH_D31H | |
| | P52aK::Vk_I30H, hz13- | |
| | 5 VH_D31H 134H | |
| | P52aK::Vk_I30H, hz13- | |
| | 5 VH_D31H 134H | |
| | H35K P52aK::Vk_I30H, | |
| | hz13-5 VH_D31H 134H | |
| | H35K::Vk_I30H, hz13- | |
| | 5 VH_D31H | |
| | 134H::Vk_I30H, hz13-5 | |
| | VH_D31H H35K | |
| | P52aK::Vk_I30H, hz13- | |
| | 5 VH_D31H | |
| | H35K::Vk_I30H, hz13-5 | |
| | VH_D31H::Vk_I30H | |
| 174 | hIgG1 HC constant | ASTKGPSVFPLAPSSKSTSGGTAALGCLVKDYFPEPVTVSWNSGALTSG |
| | region | VHTFPAVLQSSGLYSLSSVVTVPSSSLGTQTYICNVNHKPSNTKVDKRV |
| | | EPKSCDKTHTCPPCPAPELLGGPSVFLFPPKPKDTLMISRTPEVTCVVV |
| | | DVSHEDPEVKFNWYVDGVEVHNAKTKPREEQYNSTYRVVSVLTVLHQDW |
| | | LNGKEYKCKVSNKALPAPIEKTISKAKGQPREPQVYTLPPSREEMTKNQ |
| | | VSLTCLVKGFYPSDIAVEWESNGQPENNYKTTPPVLDSDGSFFLYSKLT |
| | | VDKSRWQQGNVFSCSVMHEALHNHYTQKSLSLSPGK |
| 175 | hIgG1 HC constant | GCGTCGACCAAGGGCCCATCCGTCTTCCCCCTGGCACCCTCCTCCAAGA |
| | region DNA | GCACCTCTGGGGGCACAGCGGCCCTGGGCTGCCTGGTCAAGGACTACTT |
| | | CCCCGAACCGGTGACGGTGTCGTGGAACTCAGGCGCCCTGACCAGCGGC |
| | | GTGCACACCTTCCCGGCTGTCCTACAGTCCTCAGGACTCTACTCCCTCA |
| | | GCAGCGTGGTGACCGTGCCCTCCAGCAGCTTGGGCACCCAGACCTACAT |
| | | CTGCAACGTGAATCACAAGCCCAGCAACACCAAGGTGGACAAGAGAGTT |
| | | GAGCCCAAATCTTGTGACAAAACTCACACATGCCCACCGTGCCCAGCAC |
| | | CTGAACTCCTGGGGGGACCGTCAGTCTTCCTCTTCCCCCCAAAACCCAA |
| | | GGACACCCTCATGATCTCCCGGACCCCTGAGGTCACATGCGTGGTGGTG |
| | | GACGTGAGCCACGAAGACCCTGAGGTCAAGTTCAACTGGTACGTGGACG |
| | | GCGTGGAGGTGCATAATGCCAAGACAAAGCCGCGGGAGGAGCAGTACAA |
| | | CAGCACGTACCGTGTGGTCAGCGTCCTCACCGTCCTGCACCAGGACTGG |
| | | CTGAATGGCAAGGAGTACAAGTGCAAGGTCTCCAACAAAGCCCTCCCAG |
| | | CCCCCATCGAGAAAACCATCTCCAAAGCCAAAGGGCAGCCCCGAGAACC |
| | | ACAGGTGTACACCCTGCCCCCATCCCGGGAGGAGATGACCAAGAACCAG |
| | | GTCAGCCTGACCTGCCTGGTCAAAGGCTTCTATCCCAGCGACATCGCCG |
| | | TGGAGTGGGAGAGCAATGGGCAGCCGGAGAACAACTACAAGACCACGCC |
| | | TCCCGTGCTGGACTCCGACGGCTCCTTCTTCCTCTATAGCAAGCTCACC |
| | | GTGGACAAGAGCAGGTGGCAGCAGGGGAACGTCTTCTCATGCTCCGTGA |
| | | TGCATGAGGCTCTGCACAACCACTACACGCAGAAGAGCCTCTCCCTGTC |
| | | CCCGGGTAAG |
| 176 | hIgG1 HC constant | ASTKGPSVFPLAPSSKSTSGGTAALGCLVKDYFPEPVTVSWNSGALTSG |
| | region without C- | VHTFPAVLQSSGLYSLSSVVTVPSSSLGTQTYICNVNHKPSNTKVDKRV |
| | terminal lysine | EPKSCDKTHTCPPCPAPELLGGPSVFLFPPKPKDTLMISRTPEVTCVVV |
| | | DVSHEDPEVKFNWYVDGVEVHNAKTKPREEQYNSTYRVVSVLTVLHQDW |
| | | LNGKEYKCKVSNKALPAPIEKTISKAKGQPREPQVYTLPPSREEMTKNQ |
| | | VSLTCLVKGFYPSDIAVEWESNGQPENNYKTTPPVLDSDGSFFLYSKLT |
| | | VDKSRWQQGNVFSCSVMHEALHNHYTQKSLSLSPG |
| 177 | hIgG1 HC constant | GCGTCGACCAAGGGCCCATCCGTCTTCCCCCTGGCACCCTCCTCCAAGA |
| | region DNA without C- | GCACCTCTGGGGGCACAGCGGCCCTGGGCTGCCTGGTCAAGGACTACTT |
| | terminal lysine | CCCCGAACCGGTGACGGTGTCGTGGAACTCAGGCGCCCTGACCAGCGGC |
| | | GTGCACACCTTCCCGGCTGTCCTACAGTCCTCAGGACTCTACTCCCTCA |
| | | GCAGCGTGGTGACCGTGCCCTCCAGCAGCTTGGGCACCCAGACCTACAT |
| | | CTGCAACGTGAATCACAAGCCCAGCAACACCAAGGTGGACAAGAGAGTT |
| | | GAGCCCAAATCTTGTGACAAAACTCACACATGCCCACCGTGCCCAGCAC |
| | | CTGAACTCCTGGGGGGACCGTCAGTCTTCCTCTTCCCCCCAAAACCCAA |
| | | GGACACCCTCATGATCTCCCGGACCCCTGAGGTCACATGCGTGGTGGTG |
| | | GACGTGAGCCACGAAGACCCTGAGGTCAAGTTCAACTGGTACGTGGACG |
| | | GCGTGGAGGTGCATAATGCCAAGACAAAGCCGCGGGAGGAGCAGTACAA |
| | | CAGCACGTACCGTGTGGTCAGCGTCCTCACCGTCCTGCACCAGGACTGG |
| | | CTGAATGGCAAGGAGTACAAGTGCAAGGTCTCCAACAAAGCCCTCCCAG |
| | | CCCCCATCGAGAAAACCATCTCCAAAGCCAAAGGGCAGCCCCGAGAACC |
| | | ACAGGTGTACACCCTGCCCCCATCCCGGGAGGAGATGACCAAGAACCAG |
| | | GTCAGCCTGACCTGCCTGGTCAAAGGCTTCTATCCCAGCGACATCGCCG |
| | | TGGAGTGGGAGAGCAATGGGCAGCCGGAGAACAACTACAAGACCACGCC |
| | | TCCCGTGCTGGACTCCGACGGCTCCTTCTTCCTCTATAGCAAGCTCACC |
| | | GTGGACAAGAGCAGGTGGCAGCAGGGGAACGTCTTCTCATGCTCCGTGA |
| | | TGCATGAGGCTCTGCACAACCACTACACGCAGAAGAGCCTCTCCCTGTC |
| | | CCCGGGT |
| 178 | hIgG1.3f HC constant | ASTKGPSVFPLAPSSKSTSGGTAALGCLVKDYFPEPVTVSWNSGALTSG |
| | region; IgG 1.3 | VHTFPAVLQSSGLYSLSSVVTVPSSSLGTQTYICNVNHKPSNTKVDKRV |
| | comprises mutations | EPKSCDKTHTCPPCPAPEAEGAPSVFLFPPKPKDTLMISRTPEVTCVVV |
| | L234A, L235E, and | DVSHEDPEVKFNWYVDGVEVHNAKTKPREEQYNSTYRVVSVLTVLHQDW |
| | G237A, shown here in | LNGKEYKCKVSNKALPAPIEKTISKAKGQPREPQVYTLPPSREEMTKNQ |
| | bold and underlining. | VSLTCLVKGFYPSDIAVEWESNGQPENNYKTTPPVLDSDGSFFLYSKLT |
| | | VDKSRWQQGNVFSCSVMHEALHNHYTQKSLSLSPGK |
| 179 | hIgG1.3f HC constant | GCGTCGACCAAGGGCCCATCGGTCTTCCCCCTGGCACCCTCCTCCAAGA |
| | region DNA sequence | GCACCTCTGGGGGCACAGCGGCCCTGGGCTGCCTGGTCAAGGACTACTT |
| | | CCCCGAACCGGTGACGGTGTCGTGGAACTCAGGCGCCCTGACCAGCGGC |
| | | GTGCACACCTTCCCGGCTGTCCTACAGTCCTCAGGACTCTACTCCCTCA |
| | | GCAGCGTGGTGACCGTGCCCTCCAGCAGCTTGGGCACCCAGACCTACAT |
| | | CTGCAACGTGAATCACAAGCCCAGCAACACCAAGGTGGACAAGAGAGTT |
| | | GAGCCCAAATCTTGTGACAAAACTCACACATGCCCACCGTGCCCAGCAC |
| | | CTGAAGCCGAAGGGGCCCCGTCAGTCTTCCTCTTCCCCCCAAAACCCAA |
| | | GGACACCCTCATGATCTCCCGGACCCCTGAGGTCACATGCGTGGTGGTG |
| | | GACGTGAGCCACGAAGACCCTGAGGTCAAGTTCAACTGGTACGTGGACG |
| | | GCGTGGAGGTGCATAATGCCAAGACAAAGCCGCGGGAGGAGCAGTACAA |
| | | CAGCACGTACCGTGTGGTCAGCGTCCTCACCGTCCTGCACCAGGACTGG |
| | | CTGAATGGCAAGGAGTACAAGTGCAAGGTCTCCAACAAAGCCCTCCCAG |
| | | CCCCCATCGAGAAAACCATCTCCAAAGCCAAAGGGCAGCCCCGAGAACC |
| | | ACAGGTGTACACCCTGCCCCCATCCCGGGAGGAGATGACCAAGAACCAG |
| | | GTCAGCCTGACCTGCCTGGTCAAAGGCTTCTATCCCAGCGACATCGCCG |
| | | TGGAGTGGGAGAGCAATGGGCAGCCGGAGAACAACTACAAGACCACGCC |
| | | TCCCGTGCTGGACTCCGACGGCTCCTTCTTCCTCTATAGCAAGCTCACC |
| | | GTGGACAAGAGCAGGTGGCAGCAGGGGAACGTCTTCTCATGCTCCGTGA |
| | | TGCATGAGGCTCTGCACAACCACTACACGCAGAAGAGCCTCTCCCTGTC |
| | | CCCGGGTAAG |
| 180 | hIgG1.3f HC constant | ASTKGPSVFPLAPSSKSTSGGTAALGCLVKDYFPEPVTVSWNSGALTSG |
| | region without the C- | VHTFPAVLQSSGLYSLSSVVTVPSSSLGTQTYICNVNHKPSNTKVDKRV |
| | terminal lysine; IgG 1.3 | EPKSCDKTHTCPPCPAPEAEGAPSVFLFPPKPKDTLMISRTPEVTCVVV |
| | mutations L234A, | DVSHEDPEVKFNWYVDGVEVHNAKTKPREEQYNSTYRVVSVLTVLHQDW |
| | L235E, and G237A are | LNGKEYKCKVSNKALPAPIEKTISKAKGQPREPQVYTLPPSREEMTKNQ |
| | shown here in bold and | VSLTCLVKGFYPSDIAVEWESNGQPENNYKTTPPVLDSDGSFFLYSKLT |
| | underlining. | VDKSRWQQGNVFSCSVMHEALHNHYTQKSLSLSPG |
| 181 | hIgG1.3f HC constant | GCGTCGACCAAGGGCCCATCGGTCTTCCCCCTGGCACCCTCCTCCAAGA |
| | region without the C- | GCACCTCTGGGGGCACAGCGGCCCTGGGCTGCCTGGTCAAGGACTACTT |
| | terminal lysine; DNA | CCCCGAACCGGTGACGGTGTCGTGGAACTCAGGCGCCCTGACCAGCGGC |
| | sequence | GTGCACACCTTCCCGGCTGTCCTACAGTCCTCAGGACTCTACTCCCTCA |
| | | GCAGCGTGGTGACCGTGCCCTCCAGCAGCTTGGGCACCCAGACCTACAT |
| | | CTGCAACGTGAATCACAAGCCCAGCAACACCAAGGTGGACAAGAGAGTT |
| | | GAGCCCAAATCTTGTGACAAAACTCACACATGCCCACCGTGCCCAGCAC |
| | | CTGAAGCCGAAGGGGCCCCGTCAGTCTTCCTCTTCCCCCCAAAACCCAA |
| | | GGACACCCTCATGATCTCCCGGACCCCTGAGGTCACATGCGTGGTGGTG |
| | | GACGTGAGCCACGAAGACCCTGAGGTCAAGTTCAACTGGTACGTGGACG |
| | | GCGTGGAGGTGCATAATGCCAAGACAAAGCCGCGGGAGGAGCAGTACAA |
| | | CAGCACGTACCGTGTGGTCAGCGTCCTCACCGTCCTGCACCAGGACTGG |
| | | CTGAATGGCAAGGAGTACAAGTGCAAGGTCTCCAACAAAGCCCTCCCAG |
| | | CCCCCATCGAGAAAACCATCTCCAAAGCCAAAGGGCAGCCCCGAGAACC |
| | | ACAGGTGTACACCCTGCCCCCATCCCGGGAGGAGATGACCAAGAACCAG |
| | | GTCAGCCTGACCTGCCTGGTCAAAGGCTTCTATCCCAGCGACATCGCCG |
| | | TGGAGTGGGAGAGCAATGGGCAGCCGGAGAACAACTACAAGACCACGCC |
| | | TCCCGTGCTGGACTCCGACGGCTCCTTCTTCCTCTATAGCAAGCTCACC |
| | | GTGGACAAGAGCAGGTGGCAGCAGGGGAACGTCTTCTCATGCTCCGTGA |
| | | TGCATGAGGCTCTGCACAACCACTACACGCAGAAGAGCCTCTCCCTGTC |
| | | CCCGGGT |
| 182 | hIgGlfa L235E, P238K | ASTKGPSVFPLAPSSKSTSGGTAALGCLVKDYFPEPVTVSWNSGALTSG |
| | (LEPK) HC constant | VHTFPAVLQSSGLYSLSSVVTVPSSSLGTQTYICNVNHKPSNTKVDKRV |
| | region; substitutions | EPKSCDKTHTCPPCPAPELEGGKSVFLFPPKPKDTLMISRTPEVTCVVV |
| | shown in bold | DVSHEDPEVKFNWYVDGVEVHNAKTKPREEQYNSTYRVVSVLTVLHQDW |
| | underlining | LNGKEYKCKVSNKALPAPIEKTISKAKGQPREPQVYTLPPSRDELTKNQ |
| | | VSLTCLVKGFYPSDIAVEWESNGQPENNYKTTPPVLDSDGSFFLYSKLT |
| | | VDKSRWQQGNVFSCSVMHEALHNHYTQKSLSLSPGK |
| 183 | hIgGlfa LEPK HC | GCGTCGACCAAGGGCCCATCCGTCTTCCCCCTGGCACCCTCCTCCAAGA |
| | constant region DNA | GCACCTCTGGGGGCACAGCGGCCCTGGGCTGCCTGGTCAAGGACTACTT |
| | sequence | CCCCGAACCGGTGACGGTGTCGTGGAACTCAGGCGCCCTGACCAGCGGC |
| | | GTGCACACCTTCCCGGCTGTCCTACAGTCCTCAGGACTCTACTCCCTCA |
| | | GCAGCGTGGTGACCGTGCCCTCCAGCAGCTTGGGCACCCAGACCTACAT |
| | | CTGCAACGTGAATCACAAGCCCAGCAACACCAAGGTGGACAAGAGAGTT |
| | | GAGCCCAAATCTTGTGACAAAACTCACACATGCCCACCGTGCCCAGCAC |
| | | CTGAACTCGAGGGGGGAAAGTCAGTCTTCCTCTTCCCCCCAAAACCCAA |
| | | GGACACCCTCATGATCTCCCGGACCCCTGAGGTCACATGCGTGGTGGTG |
| | | GACGTGAGCCACGAAGACCCTGAGGTCAAGTTCAACTGGTACGTGGACG |
| | | GCGTGGAGGTGCATAATGCCAAGACAAAGCCGCGGGAGGAGCAGTACAA |
| | | CAGCACGTACCGTGTGGTCAGCGTCCTCACCGTCCTGCACCAGGACTGG |
| | | CTGAATGGCAAGGAGTACAAGTGCAAGGTCTCCAACAAAGCCCTCCCAG |
| | | CCCCCATCGAGAAAACCATCTCCAAAGCCAAAGGGCAGCCCCGAGAACC |
| | | ACAGGTGTACACCCTGCCCCCATCCCGGGATGAGCTGACCAAGAACCAG |
| | | GTCAGCCTGACCTGCCTGGTCAAAGGCTTCTATCCCAGCGACATCGCCG |
| | | TGGAGTGGGAGAGCAATGGGCAGCCGGAGAACAACTACAAGACCACGCC |
| | | TCCCGTGCTGGACTCCGACGGCTCCTTCTTCCTCTATAGCAAGCTCACC |
| | | GTGGACAAGAGCAGGTGGCAGCAGGGGAACGTCTTCTCATGCTCCGTGA |
| | | TGCATGAGGCTCTGCACAACCACTACACGCAGAAGAGCCTCTCCCTGTC |
| | | CCCGGGTAAA |
| 184 | hIgGlfa LEPK HC | ASTKGPSVFPLAPSSKSTSGGTAALGCLVKDYFPEPVTVSWNSGALTSG |
| | constant region without | VHTFPAVLQSSGLYSLSSVVTVPSSSLGTQTYICNVNHKPSNTKVDKRV |
| | the C-terminal lysine | EPKSCDKTHTCPPCPAPELEGGKSVFLFPPKPKDTLMISRTPEVTCVVV |
| | | DVSHEDPEVKFNWYVDGVEVHNAKTKPREEQYNSTYRVVSVLTVLHQDW |
| | | LNGKEYKCKVSNKALPAPIEKTISKAKGQPREPQVYTLPPSRDELTKNQ |
| | | VSLTCLVKGFYPSDIAVEWESNGQPENNYKTTPPVLDSDGSFFLYSKLT |
| | | VDKSRWQQGNVFSCSVMHEALHNHYTQKSLSLSPG |
| 185 | hIgGlfa LEPK HC | GCGTCGACCAAGGGCCCATCCGTCTTCCCCCTGGCACCCTCCTCCAAGA |
| | constant region without | GCACCTCTGGGGGCACAGCGGCCCTGGGCTGCCTGGTCAAGGACTACTT |
| | the C-terminal lysine | CCCCGAACCGGTGACGGTGTCGTGGAACTCAGGCGCCCTGACCAGCGGC |
| | DNA sequence | GTGCACACCTTCCCGGCTGTCCTACAGTCCTCAGGACTCTACTCCCTCA |
| | | GCAGCGTGGTGACCGTGCCCTCCAGCAGCTTGGGCACCCAGACCTACAT |
| | | CTGCAACGTGAATCACAAGCCCAGCAACACCAAGGTGGACAAGAGAGTT |
| | | GAGCCCAAATCTTGTGACAAAACTCACACATGCCCACCGTGCCCAGCAC |
| | | CTGAACTCGAGGGGGGAAAGTCAGTCTTCCTCTTCCCCCCAAAACCCAA |
| | | GGACACCCTCATGATCTCCCGGACCCCTGAGGTCACATGCGTGGTGGTG |
| | | GACGTGAGCCACGAAGACCCTGAGGTCAAGTTCAACTGGTACGTGGACG |
| | | GCGTGGAGGTGCATAATGCCAAGACAAAGCCGCGGGAGGAGCAGTACAA |
| | | CAGCACGTACCGTGTGGTCAGCGTCCTCACCGTCCTGCACCAGGACTGG |
| | | CTGAATGGCAAGGAGTACAAGTGCAAGGTCTCCAACAAAGCCCTCCCAG |
| | | CCCCCATCGAGAAAACCATCTCCAAAGCCAAAGGGCAGCCCCGAGAACC |
| | | ACAGGTGTACACCCTGCCCCCATCCCGGGATGAGCTGACCAAGAACCAG |
| | | GTCAGCCTGACCTGCCTGGTCAAAGGCTTCTATCCCAGCGACATCGCCG |
| | | TGGAGTGGGAGAGCAATGGGCAGCCGGAGAACAACTACAAGACCACGCC |
| | | TCCCGTGCTGGACTCCGACGGCTCCTTCTTCCTCTATAGCAAGCTCACC |
| | | GTGGACAAGAGCAGGTGGCAGCAGGGGAACGTCTTCTCATGCTCCGTGA |
| | | TGCATGAGGCTCTGCACAACCACTACACGCAGAAGAGCCTCTCCCTGTC |
| | | CCCGGGT |
| 186 | hIgG2.3 HC constant | ASTKGPSVFPLAPCSRSTSESTAALGCLVKDYFPEPVTVSWNSGALTSG |
| | region | VHTFPAVLQSSGLYSLSSVVTVPSSNFGTQTYTCNVDHKPSNTKVDKTV |
| | | ERKSCVECPPCPAPPVAGPSVFLFPPKPKDTLMISRTPEVTCVVVDVSH |
| | | EDPEVQFNWYVDGVEVHNAKTKPREEQFNSTFRVVSVLTVVHQDWLNGK |
| | | EYKCKVSNKGLPAPIEKTISKTKGQPREPQVYTLPPSREEMTKNQVSLT |
| | | CLVKGFYPSDIAVEWESNGQPENNYKTTPPMLDSDGSFFLYSKLTVDKS |
| | | RWQQGNVFSCSVMHEALHNHYTQKSLSLSPGK |
| 187 | hIgG2.3 HC constant | GCGTCGACCAAGGGCCCCTCTGTGTTTCCTCTGGCCCCTTGCTCCCGGT |
| | region DNA | CCACCTCTGAGTCTACCGCTGCTCTGGGCTGCCTGGTCAAGGACTACTT |
| | | CCCCGAGCCTGTGACCGTGTCCTGGAACTCTGGCGCTCTGACCTCCGGC |
| | | GTGCACACCTTTCCAGCCGTGCTGCAGTCCTCCGGCCTGTACTCTCTGT |
| | | CCTCCGTCGTGACCGTGCCCTCCTCCAACTTCGGCACCCAGACCTACAC |
| | | CTGTAACGTGGACCACAAGCCCTCCAACACCAAGGTGGACAAGACCGTG |
| | | GAACGGAAGTCCTGCGTGGAATGCCCTCCTTGCCCTGCACCTCCTGTGG |
| | | CTGGCAAATCCGTGTTCCTGTTCCCCCCAAAGCCCAAGGACACCCTGAT |
| | | GATCTCCCGGACCCCCGAAGTGACCTGCGTGGTGGTGGATGTGTCCCAC |
| | | GAGGACCCCGAGGTGCAGTTCAATTGGTACGTGGACGGCGTGGAAGTGC |
| | | ACAACGCCAAGACCAAGCCCAGAGAGGAACAGTTCAACTCCACCTTCCG |
| | | GGTGGTGTCCGTGCTGACCGTGGTGCACCAGGACTGGCTGAACGGCAAA |
| | | GAGTACAAGTGCAAGGTGTCCAACAAGGGCCTGCCTGCCCCCATCGAAA |
| | | AGACCATCTCCAAGACAAAGGGCCAGCCCCGCGAGCCTCAGGTGTACAC |
| | | ACTGCCTCCCAGCCGGGAAGAGATGACCAAGAACCAGGTGTCCCTGACC |
| | | TGTCTGGTCAAGGGCTTCTACCCCTCCGATATCGCCGTGGAATGGGAGT |
| | | CCAACGGCCAGCCCGAGAACAACTACAAGACCACCCCCCCCATGCTGGA |
| | | CTCCGACGGCTCATTCTTCCTGTACTCCAAGCTGACAGTGGACAAGTCC |
| | | CGGTGGCAGCAGGGCAACGTGTTCTCCTGCTCTGTGATGCACGAGGCCC |
| | | TGCACAACCACTACACCCAGAAGTCCCTGTCCCTGAGCCCCGGCAAA |
| 188 | hIgG2.3 HC constant | ASTKGPSVFPLAPCSRSTSESTAALGCLVKDYFPEPVTVSWNSGALTSG |
| | region, without C- | VHTFPAVLQSSGLYSLSSVVTVPSSNFGTQTYTCNVDHKPSNTKVDKTV |
| | terminal lysine | ERKSCVECPPCPAPPVAGPSVFLFPPKPKDTLMISRTPEVTCVVVDVSH |
| | | EDPEVQFNWYVDGVEVHNAKTKPREEQFNSTFRVVSVLTVVHQDWLNGK |
| | | EYKCKVSNKGLPAPIEKTISKTKGQPREPQVYTLPPSREEMTKNQVSLT |
| | | CLVKGFYPSDIAVEWESNGQPENNYKTTPPMLDSDGSFFLYSKLTVDKS |
| | | RWQQGNVFSCSVMHEALHNHYTQKSLSLSPG |
| 189 | hIgG2.3 HC constant | GCGTCGACCAAGGGCCCCTCTGTGTTTCCTCTGGCCCCTTGCTCCCGGT |
| | region, without C- | CCACCTCTGAGTCTACCGCTGCTCTGGGCTGCCTGGTCAAGGACTACTT |
| | terminal lysine DNA | CCCCGAGCCTGTGACCGTGTCCTGGAACTCTGGCGCTCTGACCTCCGGC |
| | | GTGCACACCTTTCCAGCCGTGCTGCAGTCCTCCGGCCTGTACTCTCTGT |
| | | CCTCCGTCGTGACCGTGCCCTCCTCCAACTTCGGCACCCAGACCTACAC |
| | | CTGTAACGTGGACCACAAGCCCTCCAACACCAAGGTGGACAAGACCGTG |
| | | GAACGGAAGTCCTGCGTGGAATGCCCTCCTTGCCCTGCACCTCCTGTGG |
| | | CTGGCAAATCCGTGTTCCTGTTCCCCCCAAAGCCCAAGGACACCCTGAT |
| | | GATCTCCCGGACCCCCGAAGTGACCTGCGTGGTGGTGGATGTGTCCCAC |
| | | GAGGACCCCGAGGTGCAGTTCAATTGGTACGTGGACGGCGTGGAAGTGC |
| | | ACAACGCCAAGACCAAGCCCAGAGAGGAACAGTTCAACTCCACCTTCCG |
| | | GGTGGTGTCCGTGCTGACCGTGGTGCACCAGGACTGGCTGAACGGCAAA |
| | | GAGTACAAGTGCAAGGTGTCCAACAAGGGCCTGCCTGCCCCCATCGAAA |
| | | AGACCATCTCCAAGACAAAGGGCCAGCCCCGCGAGCCTCAGGTGTACAC |
| | | ACTGCCTCCCAGCCGGGAAGAGATGACCAAGAACCAGGTGTCCCTGACC |
| | | TGTCTGGTCAAGGGCTTCTACCCCTCCGATATCGCCGTGGAATGGGAGT |
| | | CCAACGGCCAGCCCGAGAACAACTACAAGACCACCCCCCCCATGCTGGA |
| | | CTCCGACGGCTCATTCTTCCTGTACTCCAAGCTGACAGTGGACAAGTCC |
| | | CGGTGGCAGCAGGGCAACGTGTTCTCCTGCTCTGTGATGCACGAGGCCC |
| | | TGCACAACCACTACACCCAGAAGTCCCTGTCCCTGAGCCCCGGC |
| 190 | hIgG2.3 P238K HC | ASTKGPSVFPLAPCSRSTSESTAALGCLVKDYFPEPVTVSWNSGALTSG |
| | constant region | VHTFPAVLQSSGLYSLSSVVTVPSSNFGTQTYTCNVDHKPSNTKVDKTV |
| | | ERKSCVECPPCPAPPVAGKSVFLFPPKPKDTLMISRTPEVTCVVVDVSH |
| | | EDPEVQFNWYVDGVEVHNAKTKPREEQFNSTFRVVSVLTVVHQDWLNGK |
| | | EYKCKVSNKGLPAPIEKTISKTKGQPREPQVYTLPPSREEMTKNQVSLT |
| | | CLVKGFYPSDIAVEWESNGQPENNYKTTPPMLDSDGSFFLYSKLTVDKS |
| | | RWQQGNVFSCSVMHEALHNHYTQKSLSLSPGK |
| 191 | hIgG2.3 P238K HC | GCGTCGACCAAGGGCCCCTCTGTGTTTCCTCTGGCCCCTTGCTCCCGGT |
| | constant region DNA | CCACCTCTGAGTCTACCGCTGCTCTGGGCTGCCTGGTCAAGGACTACTT |
| | sequence | CCCCGAGCCTGTGACCGTGTCCTGGAACTCTGGCGCTCTGACCTCCGGC |
| | | GTGCACACCTTTCCAGCCGTGCTGCAGTCCTCCGGCCTGTACTCTCTGT |
| | | CCTCCGTCGTGACCGTGCCCTCCTCCAACTTCGGCACCCAGACCTACAC |
| | | CTGTAACGTGGACCACAAGCCCTCCAACACCAAGGTGGACAAGACCGTG |
| | | GAACGGAAGTCCTGCGTGGAATGCCCTCCTTGCCCTGCACCTCCTGTGG |
| | | CTGGCAAATCCGTGTTCCTGTTCCCCCCAAAGCCCAAGGACACCCTGAT |
| | | GATCTCCCGGACCCCCGAAGTGACCTGCGTGGTGGTGGATGTGTCCCAC |
| | | GAGGACCCCGAGGTGCAGTTCAATTGGTACGTGGACGGCGTGGAAGTGC |
| | | ACAACGCCAAGACCAAGCCCAGAGAGGAACAGTTCAACTCCACCTTCCG |
| | | GGTGGTGTCCGTGCTGACCGTGGTGCACCAGGACTGGCTGAACGGCAAA |
| | | GAGTACAAGTGCAAGGTGTCCAACAAGGGCCTGCCTGCCCCCATCGAAA |
| | | AGACCATCTCCAAGACAAAGGGCCAGCCCCGCGAGCCTCAGGTGTACAC |
| | | ACTGCCTCCCAGCCGGGAAGAGATGACCAAGAACCAGGTGTCCCTGACC |
| | | TGTCTGGTCAAGGGCTTCTACCCCTCCGATATCGCCGTGGAATGGGAGT |
| | | CCAACGGCCAGCCCGAGAACAACTACAAGACCACCCCCCCCATGCTGGA |
| | | CTCCGACGGCTCATTCTTCCTGTACTCCAAGCTGACAGTGGACAAGTCC |
| | | CGGTGGCAGCAGGGCAACGTGTTCTCCTGCTCTGTGATGCACGAGGCCC |
| | | TGCACAACCACTACACCCAGAAGTCCCTGTCCCTGAGCCCCGGCAAA |
| 192 | hIgG2.3 P238K HC | ASTKGPSVFPLAPCSRSTSESTAALGCLVKDYFPEPVTVSWNSGALTSG |
| | constant region without | VHTFPAVLQSSGLYSLSSVVTVPSSNFGTQTYTCNVDHKPSNTKVDKTV |
| | the C-terminal lysine | ERKSCVECPPCPAPPVAGKSVFLFPPKPKDTLMISRTPEVTCVVVDVSH |
| | | EDPEVQFNWYVDGVEVHNAKTKPREEQFNSTFRVVSVLTVVHQDWLNGK |
| | | EYKCKVSNKGLPAPIEKTISKTKGQPREPQVYTLPPSREEMTKNQVSLT |
| | | CLVKGFYPSDIAVEWESNGQPENNYKTTPPMLDSDGSFFLYSKLTVDKS |
| | | RWQQGNVFSCSVMHEALHNHYTQKSLSLSPG |
| 193 | hIgG2.3 P238K HC | GCGTCGACCAAGGGCCCCTCTGTGTTTCCTCTGGCCCCTTGCTCCCGGT |
| | constant region without | CCACCTCTGAGTCTACCGCTGCTCTGGGCTGCCTGGTCAAGGACTACTT |
| | the C-terminal lysine | CCCCGAGCCTGTGACCGTGTCCTGGAACTCTGGCGCTCTGACCTCCGGC |
| | DNA sequence | GTGCACACCTTTCCAGCCGTGCTGCAGTCCTCCGGCCTGTACTCTCTGT |
| | | CCTCCGTCGTGACCGTGCCCTCCTCCAACTTCGGCACCCAGACCTACAC |
| | | CTGTAACGTGGACCACAAGCCCTCCAACACCAAGGTGGACAAGACCGTG |
| | | GAACGGAAGTCCTGCGTGGAATGCCCTCCTTGCCCTGCACCTCCTGTGG |
| | | CTGGCAAATCCGTGTTCCTGTTCCCCCCAAAGCCCAAGGACACCCTGAT |
| | | GATCTCCCGGACCCCCGAAGTGACCTGCGTGGTGGTGGATGTGTCCCAC |
| | | GAGGACCCCGAGGTGCAGTTCAATTGGTACGTGGACGGCGTGGAAGTGC |
| | | ACAACGCCAAGACCAAGCCCAGAGAGGAACAGTTCAACTCCACCTTCCG |
| | | GGTGGTGTCCGTGCTGACCGTGGTGCACCAGGACTGGCTGAACGGCAAA |
| | | GAGTACAAGTGCAAGGTGTCCAACAAGGGCCTGCCTGCCCCCATCGAAA |
| | | AGACCATCTCCAAGACAAAGGGCCAGCCCCGCGAGCCTCAGGTGTACAC |
| | | ACTGCCTCCCAGCCGGGAAGAGATGACCAAGAACCAGGTGTCCCTGACC |
| | | TGTCTGGTCAAGGGCTTCTACCCCTCCGATATCGCCGTGGAATGGGAGT |
| | | CCAACGGCCAGCCCGAGAACAACTACAAGACCACCCCCCCCATGCTGGA |
| | | CTCCGACGGCTCATTCTTCCTGTACTCCAAGCTGACAGTGGACAAGTCC |
| | | CGGTGGCAGCAGGGCAACGTGTTCTCCTGCTCTGTGATGCACGAGGCCC |
| | | TGCACAACCACTACACCCAGAAGTCCCTGTCCCTGAGCCCCGGC |
| 194 | Exemplary LC constant | RTVAAPSVFIFPPSDEQLKSGTASVVCLLNNFYPREAKVQWKVDNALQS |
| | region | GNSQESVTEQDSKDSTYSLSSTLTLSKADYEKHKVYACEVTHQGLSSPV |
| | | TKSFNRGEC |
| 195 | Exemplary LC constant | CGTACGGTGGCTGCACCATCTGTCTTCATCTTCCCGCCATCTGATGAGC |
| | region DNA sequence | AGTTGAAATCTGGAACTGCCTCTGTTGTGTGCCTGCTGAATAACTTCTA |
| | | TCCCAGAGAGGCCAAAGTACAGTGGAAGGTGGATAACGCCCTCCAATCG |
| | | GGTAACTCCCAGGAGAGTGTCACAGAGCAGGACAGCAAGGACAGCACCT |
| | | ACAGCCTCAGCAGCACCCTGACGCTGAGCAAAGCAGACTACGAGAAACA |
| | | CAAAGTCTACGCCTGCGAAGTCACCCATCAGGGCCTGAGCTCGCCCGTC |
| | | ACAAAGAGCTTCAACAGGGGAGAGTGT |
| 196 | hz13-5 D31E HC | EVLLVQSGAEVKKPGASVKLSCTSSDFNIKEHFIHWVRQAPGKGLEWMG |
| | | RIDPEDDETKYAPKFQDRVTMTADTSTDTAYMELSSLRSEDTAVFYCAG |
| | | YGNYEGAMDYWGQGTLVTVSSASTKGPSVFPLAPSSKSTSGGTAALGCL |
| | | VKDYFPEPVTVSWNSGALTSGVHTFPAVLQSSGLYSLSSVVTVPSSSLG |
| | | TQTYICNVNHKPSNTKVDKRVEPKSCDKTHTCPPCPAPEAEGAPSVFLF |
| | | PPKPKDTLMISRTPEVTCVVVDVSHEDPEVKFNWYVDGVEVHNAKTKPR |
| | | EEQYNSTYRVVSVLTVLHQDWLNGKEYKCKVSNKALPAPIEKTISKAKG |
| | | QPREPQVYTLPPSREEMTKNQVSLTCLVKGFYPSDIAVEWESNGQPENN |
| | | YKTTPPVLDSDGSFFLYSKLTVDKSRWQQGNVFSCSVMHEALHNHYTQK |
| | | SLSLSPGK |
| 197 | hz13-5 D31E HC DNA | GAAGTGCTGCTGGTGCAGTCTGGCGCTGAGGTGAAGAAGCCTGGAGCTA |
| | sequence | GCGTGAAGCTGTCCTGCACCTCCTCCGACTTCAACATCAAAGAGCACTT |
| | | CATCCACTGGGTGCGGCAGGCCCCTGGCAAGGGCCTGGAATGGATGGGC |
| | | CGGATCGACCCTGAGGACGACGAGACCAAGTACGCCCCCAAGTTTCAAG |
| | | ATAGAGTGACCATGACCGCCGATACCTCTACCGACACAGCCTACATGGA |
| | | ACTGAGCTCTCTGAGATCCGAGGATACAGCTGTGTTCTATTGTGCTGGC |
| | | TACGGCAACTACGAGGGAGCCATGGACTACTGGGGCCAGGGCACCCTCG |
| | | TCACCGTGTCCTCTGCGTCGACCAAGGGCCCATCGGTCTTCCCCCTGGC |
| | | ACCCTCCTCCAAGAGCACCTCTGGGGGCACAGCGGCCCTGGGCTGCCTG |
| | | GTCAAGGACTACTTCCCCGAACCGGTGACGGTGTCGTGGAACTCAGGCG |
| | | CCCTGACCAGCGGCGTGCACACCTTCCCGGCTGTCCTACAGTCCTCAGG |
| | | ACTCTACTCCCTCAGCAGCGTGGTGACCGTGCCCTCCAGCAGCTTGGGC |
| | | ACCCAGACCTACATCTGCAACGTGAATCACAAGCCCAGCAACACCAAGG |
| | | TGGACAAGAGAGTTGAGCCCAAATCTTGTGACAAAACTCACACATGCCC |
| | | ACCGTGCCCAGCACCTGAAGCCGAAGGGGCCCCGTCAGTCTTCCTCTTC |
| | | CCCCCAAAACCCAAGGACACCCTCATGATCTCCCGGACCCCTGAGGTCA |
| | | CATGCGTGGTGGTGGACGTGAGCCACGAAGACCCTGAGGTCAAGTTCAA |
| | | CTGGTACGTGGACGGCGTGGAGGTGCATAATGCCAAGACAAAGCCGCGG |
| | | GAGGAGCAGTACAACAGCACGTACCGTGTGGTCAGCGTCCTCACCGTCC |
| | | TGCACCAGGACTGGCTGAATGGCAAGGAGTACAAGTGCAAGGTCTCCAA |
| | | CAAAGCCCTCCCAGCCCCCATCGAGAAAACCATCTCCAAAGCCAAAGGG |
| | | CAGCCCCGAGAACCACAGGTGTACACCCTGCCCCCATCCCGGGAGGAGA |
| | | TGACCAAGAACCAGGTCAGCCTGACCTGCCTGGTCAAAGGCTTCTATCC |
| | | CAGCGACATCGCCGTGGAGTGGGAGAGCAATGGGCAGCCGGAGAACAAC |
| | | TACAAGACCACGCCTCCCGTGCTGGACTCCGACGGCTCCTTCTTCCTCT |
| | | ATAGCAAGCTCACCGTGGACAAGAGCAGGTGGCAGCAGGGGAACGTCTT |
| | | CTCATGCTCCGTGATGCATGAGGCTCTGCACAACCACTACACGCAGAAG |
| | | AGCCTCTCCCTGTCCCCGGGTAAA |
| 198 | hz13 -5 D31E HC, | EVLLVQSGAEVKKPGASVKLSCTSSDFNIKEHFIHWVRQAPGKGLEWMG |
| | without C-terminal lysine | RIDPEDDETKYAPKFQDRVTMTADTSTDTAYMELSSLRSEDTAVFYCAG |
| | | YGNYEGAMDYWGQGTLVTVSSASTKGPSVFPLAPSSKSTSGGTAALGCL |
| | | VKDYFPEPVTVSWNSGALTSGVHTFPAVLQSSGLYSLSSVVTVPSSSLG |
| | | TQTYICNVNHKPSNTKVDKRVEPKSCDKTHTCPPCPAPEAEGAPSVFLF |
| | | PPKPKDTLMISRTPEVTCVVVDVSHEDPEVKFNWYVDGVEVHNAKTKPR |
| | | EEQYNSTYRVVSVLTVLHQDWLNGKEYKCKVSNKALPAPIEKTISKAKG |
| | | QPREPQVYTLPPSREEMTKNQVSLTCLVKGFYPSDIAVEWESNGQPENN |
| | | YKTTPPVLDSDGSFFLYSKLTVDKSRWQQGNVFSCSVMHEALHNHYTQK |
| | | SLSLSPG |
| 199 | hz13 -5 D31E HC DNA | GAAGTGCTACTAGTGCAAAGTGGTGCAGAAGTCAAGAAGCCTGGAGCTT |
| | sequence, without C- | CCGTGAAGCTGTCCTGCACCAGCTCCGACTTCAACATCAAGGAACACTT |
| | terminal lysine | CATCCACTGGGTGCGGCAGGCCCCCGGCAAGGGCCTGGAATGGATGGGC |
| | | AGAATCGATCCTGAGGACGACGAGACCAAGTACGCCCCTAAATTTCAGG |
| | | ACAGAGTGACCATGACAGCTGATACCAGCACCGACACCGCCTACATGGA |
| | | ACTGTCTTCTCTGAGATCCGAGGATACAGCCGTGTTCTACTGTGCTGGA |
| | | TATGGCAACTACGAGGGCGCCATGGACTACTGGGGCCAGGGCACCCTGG |
| | | TGACCGTGTCCTCTGCTAGCACCAAGGGCCCATCGGTCTTCCCCCTGGC |
| | | ACCCTCCTCCAAGAGCACCTCTGGGGGCACAGCGGCCCTGGGCTGCCTG |
| | | GTCAAGGACTACTTCCCCGAACCGGTGACGGTGTCGTGGAACTCAGGCG |
| | | CCCTGACCAGCGGCGTGCACACCTTCCCGGCTGTCCTACAGTCCTCAGG |
| | | ACTCTACTCCCTCAGCAGCGTGGTGACCGTGCCCTCCAGCAGCTTGGGC |
| | | ACCCAGACCTACATCTGCAACGTGAATCACAAGCCCAGCAACACCAAGG |
| | | TGGACAAGAGAGTTGAGCCCAAATCTTGTGACAAAACTCACACATGCCC |
| | | ACCGTGCCCAGCACCTGAAGCCGAAGGGGCCCCGTCAGTCTTCCTCTTC |
| | | CCCCCAAAACCCAAGGACACCCTCATGATCTCCCGGACCCCTGAGGTCA |
| | | CATGCGTGGTGGTGGACGTGAGCCACGAAGACCCTGAGGTCAAGTTCAA |
| | | CTGGTACGTGGACGGCGTGGAGGTGCATAATGCCAAGACAAAGCCGCGG |
| | | GAGGAGCAGTACAACAGCACGTACCGTGTGGTCAGCGTCCTCACCGTCC |
| | | TGCACCAGGACTGGCTGAATGGCAAGGAGTACAAGTGCAAGGTCTCCAA |
| | | CAAAGCCCTCCCAGCCCCCATCGAGAAAACCATCTCCAAAGCCAAAGGG |
| | | CAGCCCCGAGAACCACAGGTGTACACCCTGCCCCCATCCCGGGAGGAGA |
| | | TGACCAAGAACCAGGTCAGCCTGACCTGCCTGGTCAAAGGCTTCTATCC |
| | | CAGCGACATCGCCGTGGAGTGGGAGAGCAATGGGCAGCCGGAGAACAAC |
| | | TACAAGACCACGCCTCCCGTGCTGGACTCCGACGGCTCCTTCTTCCTCT |
| | | ATAGCAAGCTCACCGTGGACAAGAGCAGGTGGCAGCAGGGGAACGTCTT |
| | | CTCATGCTCCGTGATGCATGAGGCTCTGCACAACCACTACACGCAGAAG |
| | | AGCCTCTCCCTGTCCCCGGGT |
| 200 | hz13-5 D31E LC | DIQMTQSPSSLSASVGDRVTITCRASENVNNYGIGFMNWFQQKPGKAPK |
| | | LLIYAASRRGSGVPSRFSGSGSGTDFTLTISSLQPEDFATYFCQQSKEV |
| | | PYTFGQGTKVEIKRTVAAPSVFIFPPSDEQLKSGTASVVCLLNNFYPRE |
| | | AKVQWKVDNALQSGNSQESVTEQDSKDSTYSLSSTLTLSKADYEKHKVY |
| | | ACEVTHQGLSSPVTKSFNRGEC |
| 201 | hz13-5 D31E LC DNA | GACATCCAGATGACCCAATCGCCATCGTCCCTGTCCGCATCCGTGGGAG |
| | sequence | ACAGAGTGACAATTACTTGCCGGGCCAGCGAAAACGTGAACAACTACGG |
| | | CATTGGCTTCATGAACTGGTTCCAGCAAAAGCCAGGAAAGGCCCCGAAG |
| | | CTCCTGATCTACGCCGCAAGCCGGAGAGGTTCCGGAGTGCCCTCACGGT |
| | | TCTCCGGCTCCGGATCTGGAACCGACTTCACACTGACCATTAGCAGCCT |
| | | CCAGCCTGAGGACTTCGCCACCTACTTCTGCCAACAATCCAAGGAGGTG |
| | | CCCTACACCTTTGGACAAGGCACCAAGGTCGAGATCAAGAGGACGGTGG |
| | | CGGCACCCTCCGTGTTTATCTTCCCGCCGTCCGACGAACAGCTTAAGTC |
| | | CGGCACTGCTTCGGTCGTGTGTCTGCTGAACAACTTCTATCCTCGCGAG |
| | | GCCAAAGTGCAGTGGAAGGTCGATAACGCGCTGCAGTCCGGGAATTCAC |
| | | AGGAATCAGTGACCGAACAGGATTCGAAGGACTCCACTTACTCGCTGTC |
| | | GAGCACTCTCACTCTGAGCAAGGCCGACTACGAAAAGCACAAAGTGTAC |
| | | GCCTGCGAAGTGACCCATCAGGGTTTGTCCTCCCCTGTGACCAAGAGCT |
| | | TCAACAGAGGGGAGTGC |
| 202 | hz13-12 D31E HC | EVQLVQSGAEVKKPGATVKISCTSSDFNIKEHFIHWVQQAPGKGLEWMG |
| | | RIDPEDDETKYAPKFQDRVTITADTSTDTAYMELSSLRSEDTAVYYCAG |
| | | YGNYEGAMDYWGQGTTVTVSSASTKGPSVFPLAPSSKSTSGGTAALGCL |
| | | VKDYFPEPVTVSWNSGALTSGVHTFPAVLQSSGLYSLSSVVTVPSSSLG |
| | | TQTYICNVNHKPSNTKVDKRVEPKSCDKTHTCPPCPAPEAEGAPSVFLF |
| | | PPKPKDTLMISRTPEVTCVVVDVSHEDPEVKFNWYVDGVEVHNAKTKPR |
| | | EEQYNSTYRVVSVLTVLHQDWLNGKEYKCKVSNKALPAPIEKTISKAKG |
| | | QPREPQVYTLPPSREEMTKNQVSLTCLVKGFYPSDIAVEWESNGQPENN |
| | | YKTTPPVLDSDGSFFLYSKLTVDKSRWQQGNVFSCSVMHEALHNHYTQK |
| | | SLSLSPGK |
| 203 | hz13-12 D31E HC DNA | GAGGTGCAGCTGGTGCAATCTGGCGCTGAAGTGAAGAAGCCTGGAGCTA |
| | sequence | CCGTCAAGATCTCCTGCACCTCCTCCGACTTCAACATCAAAGAGCACTT |
| | | CATCCACTGGGTGCAGCAGGCCCCTGGCAAGGGCCTGGAATGGATGGGC |
| | | CGGATCGATCCTGAGGACGACGAGACAAAGTACGCCCCCAAGTTTCAGG |
| | | ACAGAGTGACCATCACCGCTGATACATCTACCGACACCGCCTACATGGA |
| | | ACTGAGCTCTCTGCGGTCCGAGGATACCGCCGTGTACTATTGTGCTGGC |
| | | TACGGCAACTACGAGGGCGCCATGGACTACTGGGGCCAGGGCACCACCG |
| | | TGACAGTGTCCAGCGCGTCGACCAAGGGCCCATCGGTCTTCCCCCTGGC |
| | | ACCCTCCTCCAAGAGCACCTCTGGGGGCACAGCGGCCCTGGGCTGCCTG |
| | | GTCAAGGACTACTTCCCCGAACCGGTGACGGTGTCGTGGAACTCAGGCG |
| | | CCCTGACCAGCGGCGTGCACACCTTCCCGGCTGTCCTACAGTCCTCAGG |
| | | ACTCTACTCCCTCAGCAGCGTGGTGACCGTGCCCTCCAGCAGCTTGGGC |
| | | ACCCAGACCTACATCTGCAACGTGAATCACAAGCCCAGCAACACCAAGG |
| | | TGGACAAGAGAGTTGAGCCCAAATCTTGTGACAAAACTCACACATGCCC |
| | | ACCGTGCCCAGCACCTGAAGCCGAAGGGGCCCCGTCAGTCTTCCTCTTC |
| | | CCCCCAAAACCCAAGGACACCCTCATGATCTCCCGGACCCCTGAGGTCA |
| | | CATGCGTGGTGGTGGACGTGAGCCACGAAGACCCTGAGGTCAAGTTCAA |
| | | CTGGTACGTGGACGGCGTGGAGGTGCATAATGCCAAGACAAAGCCGCGG |
| | | GAGGAGCAGTACAACAGCACGTACCGTGTGGTCAGCGTCCTCACCGTCC |
| | | TGCACCAGGACTGGCTGAATGGCAAGGAGTACAAGTGCAAGGTCTCCAA |
| | | CAAAGCCCTCCCAGCCCCCATCGAGAAAACCATCTCCAAAGCCAAAGGG |
| | | CAGCCCCGAGAACCACAGGTGTACACCCTGCCCCCATCCCGGGAGGAGA |
| | | TGACCAAGAACCAGGTCAGCCTGACCTGCCTGGTCAAAGGCTTCTATCC |
| | | CAGCGACATCGCCGTGGAGTGGGAGAGCAATGGGCAGCCGGAGAACAAC |
| | | TACAAGACCACGCCTCCCGTGCTGGACTCCGACGGCTCCTTCTTCCTCT |
| | | ATAGCAAGCTCACCGTGGACAAGAGCAGGTGGCAGCAGGGGAACGTCTT |
| | | CTCATGCTCCGTGATGCATGAGGCTCTGCACAACCACTACACGCAGAAG |
| | | AGCCTCTCCCTGTCCCCGGGTAAA |
| 204 | hz13-12 D31E HC, | EVQLVQSGAEVKKPGATVKISCTSSDFNIKEHFIHWVQQAPGKGLEWMG |
| | without C-terminal lysine | RIDPEDDETKYAPKFQDRVTITADTSTDTAYMELSSLRSEDTAVYYCAG |
| | | YGNYEGAMDYWGQGTTVTVSSASTKGPSVFPLAPSSKSTSGGTAALGCL |
| | | VKDYFPEPVTVSWNSGALTSGVHTFPAVLQSSGLYSLSSVVTVPSSSLG |
| | | TQTYICNVNHKPSNTKVDKRVEPKSCDKTHTCPPCPAPEAEGAPSVFLF |
| | | PPKPKDTLMISRTPEVTCVVVDVSHEDPEVKFNWYVDGVEVHNAKTKPR |
| | | EEQYNSTYRVVSVLTVLHQDWLNGKEYKCKVSNKALPAPIEKTISKAKG |
| | | QPREPQVYTLPPSREEMTKNQVSLTCLVKGFYPSDIAVEWESNGQPENN |
| | | YKTTPPVLDSDGSFFLYSKLTVDKSRWQQGNVFSCSVMHEALHNHYTQK |
| | | SLSLSPG |
| 205 | hz13-12 D31E HC DNA | GAGGTGCAACTGGTGCAGTCGGGAGCCGAAGTCAAGAAGCCTGGAGCCA |
| | sequence, without C- | CCGTGAAGATCAGTTGCACCAGTTCCGATTTCAACATCAAGGAGCACTT |
| | terminal lysine | CATCCACTGGGTGCAACAGGCTCCGGGAAAGGGCCTCGAGTGGATGGGG |
| | | CGGATTGATCCCGAGGACGACGAAACCAAATACGCCCCGAAGTTTCAGG |
| | | ACCGGGTCACCATTACCGCCGACACCAGCACCGACACTGCGTACATGGA |
| | | ACTGTCGTCCCTGCGCTCCGAGGATACCGCCGTGTACTATTGCGCCGGT |
| | | TACGGGAACTACGAGGGTGCCATGGATTACTGGGGACAGGGAACCACCG |
| | | TGACTGTGTCCAGCGCGTCCACTAAGGGACCTTCAGTGTTCCCGCTGGC |
| | | TCCTAGCTCAAAGTCCACCTCGGGAGGCACTGCAGCTCTGGGTTGCCTC |
| | | GTGAAGGACTACTTCCCTGAACCTGTGACCGTGTCCTGGAATTCCGGCG |
| | | CTCTTACCTCCGGAGTGCATACTTTCCCCGCCGTGCTCCAAAGCTCCGG |
| | | CCTGTACAGCCTGTCCTCCGTCGTGACTGTCCCGAGCTCGTCCCTGGGA |
| | | ACCCAGACTTACATTTGCAACGTGAACCACAAGCCCTCAAACACCAAGG |
| | | TCGATAAGAGGGTGGAACCCAAGAGCTGTGACAAGACTCACACTTGCCC |
| | | GCCATGTCCAGCCCCTGAAGCCGAGGGTGCCCCATCTGTGTTTCTGTTC |
| | | CCGCCCAAACCGAAGGACACCCTGATGATCTCAAGAACCCCGGAGGTCA |
| | | CCTGTGTGGTGGTCGACGTGTCCCACGAGGATCCCGAAGTGAAATTCAA |
| | | TTGGTACGTGGACGGGGTGGAGGTCCATAACGCCAAGACAAAGCCGAGA |
| | | GAGGAGCAGTACAACTCCACGTATCGCGTTGTCTCCGTGCTGACCGTGC |
| | | TGCATCAGGACTGGCTCAACGGAAAGGAGTACAAGTGCAAAGTGTCGAA |
| | | CAAGGCGTTGCCGGCACCAATCGAAAAGACTATCTCGAAAGCGAAGGGA |
| | | CAGCCCAGGGAGCCTCAGGTCTACACACTGCCTCCAAGCCGGGAAGAAA |
| | | TGACTAAGAACCAAGTGTCGCTGACTTGCCTTGTGAAGGGCTTTTACCC |
| | | CTCCGACATTGCCGTCGAATGGGAATCCAACGGGCAGCCCGAGAACAAC |
| | | TACAAGACCACCCCGCCTGTGCTGGACAGCGACGGCAGCTTCTTCTTGT |
| | | ACTCGAAGCTGACCGTGGATAAGTCCCGGTGGCAACAGGGCAATGTGTT |
| | | CTCCTGCTCCGTGATGCACGAAGCCCTCCACAACCACTACACGCAGAAG |
| | | TCCCTGTCGCTTTCACCCGGC |
| 206 | hz13-12 D31E LC | DVVMTQSPLSLPVTLGQPASISCRASENVNNYGIGFMNWFQQRPGQSPR |
| | | LLIYAASRRGSGVPDRFSGSGSGTDFTLKISRVEEEDVGVYYCQQSKEV |
| | | PYTFGGGTKVEIKRTVAAPSVFIFPPSDEQLKSGTASVVCLLNNFYPRE |
| | | AKVQWKVDNALQSGNSQESVTEQDSKDSTYSLSSTLTLSKADYEKHKVY |
| | | ACEVTHQGLSSPVTKSFNRGEC |
| 207 | hz13-12 D31E LC DNA | GACGTCGTGATGACCCAGTCGCCCCTGTCGTTGCCCGTGACTCTGGGTC |
| | sequence | AGCCTGCCTCAATTAGCTGTAGGGCCAGCGAGAACGTGAACAACTACGG |
| | | AATCGGGTTCATGAACTGGTTCCAGCAACGCCCGGGACAAAGCCCCCGG |
| | | CTGCTGATCTACGCCGCGTCAAGACGGGGTTCAGGAGTGCCAGATCGGT |
| | | TCTCTGGTTCCGGATCGGGCACCGACTTCACGCTGAAAATCTCCCGCGT |
| | | GGAAGAGGAGGACGTGGGAGTGTATTACTGTCAGCAGTCCAAGGAAGTG |
| | | CCTTACACCTTCGGCGGCGGAACTAAGGTCGAGATTAAGAGGACCGTGG |
| | | CTGCTCCTTCCGTGTTTATCTTCCCGCCGTCCGATGAACAGCTGAAGTC |
| | | CGGGACTGCCAGCGTCGTGTGCCTGCTCAACAACTTTTACCCGCGGGAG |
| | | GCCAAAGTGCAGTGGAAGGTCGACAACGCGCTCCAATCCGGCAATAGCC |
| | | AGGAATCCGTGACCGAGCAGGACTCGAAGGATTCCACATACTCCCTGTC |
| | | CTCGACCCTTACTCTGAGCAAGGCCGACTACGAGAAGCACAAGGTCTAC |
| | | GCCTGCGAAGTGACCCATCAAGGACTTTCCAGCCCCGTGACCAAGTCCT |
| | | TCAACCGCGGCGAATGC |
| 208 | mumAb (murine | EVQLQQPGAELVKPGASVKLSCKASDHTFTTYWIHWVKQRPGRGLEWIG |
| | surrogate) VH | RIGPNSGGSKYNEKFRTKATLTVDKPSSTAYMQLSSLKSEDSAVYFCAF |
| | | SNYLDYLDYWGQGTTLTVSS |
| 209 | mumAb VH DNA | GAGGTCCAGCTGCAGCAGCCTGGGGCTGAGCTTGTGAAGCCTGGGGCTT |
| | sequence | CAGTGAAGCTGTCCTGCAAGGCTTCTGACCACACCTTCACCACCTACTG |
| | | GATACACTGGGTGAAGCAGAGGCCTGGACGAGGCCTTGAGTGGATTGGA |
| | | AGGATTGGTCCTAATAGTGGTGGTTCTAAATACAATGAGAAGTTCAGGA |
| | | CCAAGGCCACACTGACTGTAGACAAACCCTCCAGCACAGCCTACATGCA |
| | | GCTCAGCAGCCTGAAATCTGAGGACTCTGCGGTCTATTTTTGTGCCTTT |
| | | AGTAACTACTTGGACTACCTTGACTACTGGGGCCAAGGCACCACTCTCA |
| | | CAGTCTCCTCA |
| 210 | muAb VL | DIVLTQSPASLAVSLGQRATISCKASQSVDHDGEGYMNWYQQKPGQPPK |
| | | LLIYAASNLESGIPARFSGSGSGTDFTLNIHPVEEEDAATYYCQQINED |
| | | PYTFGGGTKLEIK |
| 211 | mumAb VL DNA | GACATTGTGCTCACCCAATCTCCAGCTTCTTTGGCTGTGTCTCTTGGGC |
| | sequence | AGAGGGCCACCATCTCCTGCAAGGCCAGCCAAAGTGTTGATCATGATGG |
| | | TGAAGGTTATATGAACTGGTACCAACAGAAACCAGGACAGCCACCCAAA |
| | | CTCCTCATCTATGCTGCATCCAATCTAGAATCTGGGATCCCAGCCAGGT |
| | | TTAGTGGCAGTGGGTCTGGGACAGACTTCACCCTCAACATCCATCCTGT |
| | | GGAGGAGGAGGATGCTGCAACTTATTACTGTCAGCAAATTAATGAGGAT |
| | | CCGTACACGTTCGGAGGGGGGACCAAGCTGGAAATAAAA |
| 212 | mumAb HC; mIgG1 | EVQLQQPGAELVKPGASVKLSCKASDHTFTTYWIHWVKQRPGRGLEWIG |
| | D265A | RIGPNSGGSKYNEKFRTKATLTVDKPSSTAYMQLSSLKSEDSAVYFCAF |
| | | SNYLDYLDYWGQGTTLTVSSAKTTPPSVYPLAPGSAAQTNSMVTLGCLV |
| | | KGYFPEPVTVTWNSGSLSSGVHTFPAVLQSDLYTLSSSVTVPSSTWPSE |
| | | TVTCNVAHPASSTKVDKKIVPRDCGCKPCICTVPEVSSVFIFPPKPKDV |
| | | LTITLTPKVTCVVVAISKDDPEVQFSWFVDDVEVHTAQTQPREEQFNST |
| | | FRSVSELPIMHQDWLNGKEFKCRVNSAAFPAPIEKTISKTKGRPKAPQV |
| | | YTIPPPKEQMAKDKVSLTCMITDFFPEDITVEWQWNGQPAENYKNTQPI |
| | | MDTDGSYFVYSKLNVQKSNWEAGNTFTCSVLHEGLHNHHTEKSLSHSPG |
| 213 | mumAb HC DNA | GAGGTCCAGCTGCAGCAGCCTGGGGCTGAGCTTGTGAAGCCTGGGGCTT |
| | sequence | CAGTGAAGCTGTCCTGCAAGGCTTCTGACCACACCTTCACCACCTACTG |
| | | GATACACTGGGTGAAGCAGAGGCCTGGACGAGGCCTTGAGTGGATTGGA |
| | | AGGATTGGTCCTAATAGTGGTGGTTCTAAATACAATGAGAAGTTCAGGA |
| | | CCAAGGCCACACTGACTGTAGACAAACCCTCCAGCACAGCCTACATGCA |
| | | GCTCAGCAGCCTGAAATCTGAGGACTCTGCGGTCTATTTTTGTGCCTTT |
| | | AGTAACTACTTGGACTACCTTGACTACTGGGGCCAAGGCACCACTCTCA |
| | | CAGTCTCCTCAGCTAAAACGACACCCCCATCTGTCTATCCGCTGGCCCC |
| | | TGGATCCGCTGCCCAAACTAACTCCATGGTGACCCTGGGATGCCTGGTC |
| | | AAGGGCTATTTCCCTGAGCCAGTGACAGTGACCTGGAACTCTGGCTCCC |
| | | TGTCCAGCGGTGTGCACACCTTCCCAGCTGTCCTGCAGTCTGACCTCTA |
| | | CACTCTGAGCAGCTCAGTGACTGTCCCCTCCAGCACCTGGCCCAGCGAG |
| | | ACCGTCACCTGCAACGTTGCCCACCCGGCCAGCAGCACCAAGGTGGACA |
| | | AGAAAATTGTGCCCAGGGATTGTGGTTGTAAGCCTTGCATATGTACAGT |
| | | CCCAGAAGTATCATCTGTCTTCATCTTCCCCCCAAAGCCCAAGGATGTG |
| | | CTCACCATTACTCTGACTCCTAAGGTCACGTGTGTTGTGGTAGCCATCA |
| | | GCAAGGATGATCCCGAGGTCCAGTTCAGCTGGTTTGTAGATGATGTGGA |
| | | GGTGCACACAGCTCAGACGCAACCCCGGGAGGAGCAGTTCAACAGCACT |
| | | TTCCGCTCAGTCAGTGAACTTCCCATCATGCACCAGGACTGGCTCAATG |
| | | GCAAGGAGTTCAAATGCAGGGTCAACAGTGCAGCTTTCCCTGCCCCCAT |
| | | CGAGAAAACCATCTCCAAAACCAAAGGCAGACCGAAGGCTCCACAGGTG |
| | | TACACCATTCCACCTCCCAAGGAGCAGATGGCCAAGGATAAAGTCAGTC |
| | | TGACCTGCATGATAACAGACTTCTTCCCTGAAGACATTACTGTGGAGTG |
| | | GCAGTGGAATGGGCAGCCAGCGGAGAACTACAAGAACACTCAGCCCATC |
| | | ATGGACACAGATGGCTCTTACTTCGTCTACAGCAAGCTCAATGTGCAGA |
| | | AGAGCAACTGGGAGGCAGGAAATACTTTCACCTGCTCTGTGTTACATGA |
| | | GGGCCTGCACAACCACCATACTGAGAAGAGCCTCTCCCACTCTCCTGGT |
| 214 | mumAb LC; mIgG1 | DIVLTQSPASLAVSLGQRATISCKASQSVDHDGEGYMNWYQQKPGQPPK |
| | D265A | LLIYAASNLESGIPARFSGSGSGTDFTLNIHPVEEEDAATYYCQQINED |
| | | PYTFGGGTKLEIKRADAAPTVSIFPPSSEQLTSGGASVVCFLNNFYPKD |
| | | INVKWKIDGSERQNGVLNSWTDQDSKDSTYSMSSTLTLTKDEYERHNSY |
| | | TCEATHKTSTSPIVKSFNRNEC |
| 215 | mumAb LC DNA | GACATTGTGCTCACCCAATCTCCAGCTTCTTTGGCTGTGTCTCTTGGGC |
| | sequence | AGAGGGCCACCATCTCCTGCAAGGCCAGCCAAAGTGTTGATCATGATGG |
| | | TGAAGGTTATATGAACTGGTACCAACAGAAACCAGGACAGCCACCCAAA |
| | | CTCCTCATCTATGCTGCATCCAATCTAGAATCTGGGATCCCAGCCAGGT |
| | | TTAGTGGCAGTGGGTCTGGGACAGACTTCACCCTCAACATCCATCCTGT |
| | | GGAGGAGGAGGATGCTGCAACTTATTACTGTCAGCAAATTAATGAGGAT |
| | | CCGTACACGTTCGGAGGGGGGACCAAGCTGGAAATAAAACGGGCTGATG |
| | | CTGCACCAACTGTATCCATCTTCCCACCATCCAGTGAGCAATTGACATC |
| | | TGGAGGTGCCTCAGTCGTGTGCTTCTTGAACAACTTCTACCCCAAAGAC |
| | | ATCAATGTCAAGTGGAAGATTGATGGCAGTGAACGACAAAATGGCGTCC |
| | | TGAACAGTTGGACCGATCAGGACAGCAAAGACAGCACCTACAGCATGAG |
| | | CAGCACCCTCACGTTGACCAAGGACGAGTATGAACGACATAACAGCTAT |
| | | ACCTGTGAGGCCACTCACAAGACATCAACTTCACCCATTGTCAAGAGCT |
| | | TCAACAGGAATGAGTGT |
| 216 | PAD4 assay substrate | TSTGGRQGSHH |
| 217 | Clone 13's primary HDX | FEGIKKKKQQKIKNILSNKTLREHNSF |
| | epitope | |
| 218 | Clone 13's secondary | FQEQQNEGHGEALL |
| | HDX epitope | |
| 219 | Clone 20's primary HDX | INDFFTYHIRHGEVHCGTN |
| | epitope | |
| 220 | Clone 20's secondary | CPEEENMDDQW |
| | HDX epitope | |
| 221 | Clone 13 HC paratope | FNIKDHF |
| | region 1 | |
| 222 | Clone 13 HC paratope | FYCAGYGNYEGAMDY |
| | region 2 | |
| 223 | Clone 13 LC paratope | SENVNNYGIGFM |
| | region 1 | |
| 224 | Clone 13 LC paratope | VCFLNNFY |
| | region 2 | |
| 225 | Hz13-5 D31E HC | FNIKEHF |
| | paratope region 1 | |
| 226 | PAD4 clone 13 Fab with | EVLLQQSGAELVKPGASVRLSCTSSDFNIKDHFIHWVKQRTEQGLEWIG |
| | his tag; HC; brackets [ ] | RIDPEDDETKYAPKFQDKVTITADTSSNTAYLQLSSLTSEDTAVFYCAG |
| | indicate constant region | YGNYEGAMDYWGQGTSVTVSS[AKTTPPSVYPLAPGSAAQTNSMVTLGC |
| | | LVKGYFPEPVTVTWNSGSLSSGVHTFPAVLQSDLYTLSSSVTVPSSPRP |
| | | SETVTCNVAHPASSTKVDKKIVPRDCGG]HHHHHH |
| 227 | PAD4 clone 13 Fab; HC; | EVLLQQSGAELVKPGASVRLSCTSSDFNIKDHFIHWVKQRTEQGLEWIG |
| | brackets [ ] indicate | RIDPEDDETKYAPKFQDKVTITADTSSNTAYLQLSSLTSEDTAVFYCAG |
| | constant region | YGNYEGAMDYWGQGTSVTVSS[AKTTPPSVYPLAPGSAAQTNSMVTLGC |
| | | LVKGYFPEPVTVTWNSGSLSSGVHTFPAVLQSDLYTLSSSVTVPSSPRP |
| | | SETVTCNVAHPASSTKVDKKIVPRDCGG] |
| 228 | PAD4 clone 13 Fab; LC; | DIVLTQSPTSLGVSPGQRATLSCRASENVNNYGIGFMNWFQQKPGQPPK |
| | brackets [ ] indicate | LLIYAASRRGSGVPARFSGSGSGTDFSLNIHPMEEDDTAMYFCQQSKEV |
| | constant region | PYTFGGGTNLEIR[RADAAPTVSIFPPSSEQLTSGGASVVCFLNNFYPK |
| | | DINVKWKIDGSERQNGVLNSWTDQDSKDSTYSMSSTLTLTKDEYERHNS |
| | | YTCEATHKTSTSPIVKSFNRNEC] |
| 229 | PAD4 clone 20 Fab with | QVQLQQSGAELARPGASVKLSCKATGYTFTNYGISWVKQRTGQGLEWIG |
| | his tag; HC; mIgG1 CH1 | EIYPRSGNTYHNEKFKDKATLTADKSSSTAYMEFRSLTSEDSAVYFCAR |
| | and mouse kappa | SMITTRYWYFDVWGKGTTVTVSSAKTTPPSVYPLAPGSAAQTNSMVTLG |
| | constant region | CLVKGYFPEPVTVTWNSGSLSSGVHTFPAVLQSDLYTLSSSVTVPSSPR |
| | | PSETVTCNVAHPASSTKVDKKIVPRDCGGHHHHHH |
| 230 | PAD4 clone 20 Fab; HC; | QVQLQQSGAELARPGASVKLSCKATGYTFTNYGISWVKQRTGQGLEWIG |
| | mIgG1 CH1 and mouse | EIYPRSGNTYHNEKFKDKATLTADKSSSTAYMEFRSLTSEDSAVYFCAR |
| | kappa constant region | SMITTRYWYFDVWGKGTTVTVSSAKTTPPSVYPLAPGSAAQTNSMVTLG |
| | | CLVKGYFPEPVTVTWNSGSLSSGVHTFPAVLQSDLYTLSSSVTVPSSPR |
| | | PSETVTCNVAHPASSTKVDKKIVPRDCGG |
| 231 | Clone 20 Fab; LC; | NIVLTQSPASLAVSLGQRATLSCRASESVDTYGNSFMHWYQQKPGQPPK |
| | mIgG1 CH1 and mouse | LLIYLASNLESGVPARFSGSGSRTDFTLTIDPVEADDAATYYCQQNNED |
| | kappa constant region | PLTFGAGTKLELKRADAAPTVSIFPPSSEQLTSGGASVVCFLNNFYPKD |
| | | INVKWKIDGSERQNGVLNSWTDQDSKDSTYSMSSTLTLTKDEYERHNSY |
| | | TCEATHKTSTSPIVKSFNRNEC |
| 232 | Arginine-coated linear | SHQESTRGKSKGKAAAAA |
| | peptide | |
| 233 | hz13-12 D31E VH | EVQLVQSGAEVKKPGATVKISCTSSDFNIKEHFIHWVQQAPGKGLEWMG |
| | | RIDPEDDETKYAPKFQDRVTITADTSTDTAYMELSSLRSEDTAVYYCAG |
| | | YGNYEGAMDYWGQGTTVTVSS |
| 234 | hz13-12 D31E VL | DVVMTQSPLSLPVTLGQPASISCRASENVNNYGIGFMNWFQQRPGQSPR |
| | | LLIYAASRRGSGVPDRFSGSGSGTDFTLKISRVEEEDVGVYYCQQSKEV |
| | | PYTFGGGTKVEIK |
| 235 | Human LC constant | VCLLNNFY |
| | region fragment | |
| | corresponding to Clone | |
| | 13 LC paratope region 2 | |
| 236 | Exemplary PRG4 peptide | AITTRSGQTLSK |
| | sequence (human) | |
| 237 | Exemplary FGA peptide | QFTSSTSYNRGDSTFESK |
| | sequence (human) | |
| 238 | Exemplary ITIH4 peptide | QLGLPGPPDVPDHAAYHPFRR |
| | sequence(human) | |
| 239 | Exemplary AMBP | GPCRAFIQLWAFDAVK |
| | peptide sequence | |
| | (human) | |
| 240 | Exemplary GSN peptide | ATASRGASQAGAPQGR |
| | sequence (human) | |
| 241 | Exemplary FGA peptide | ESSSHHPGIAEFPSRGK |
| | sequence (human) | |
| 242 | Exemplary C3 peptide | SGQSEDRQPVPGQQMTLK |
| | sequence (human) | |
| 243 | Exemplary C3 peptide | ASHLGLARSNLDEDIIAEENIVSR |
| | sequence (human) | |
| 244 | Exemplary A2M peptide | VGFYESDVMGRGHAR |
| | sequence (human) | |
| 245 | Exemplary HP peptide | LRTEGDGVYTLNDK |
| | sequence (human) | |
| 246 | Exemplary TF peptide | DLLFRDDTVCLAK |
| | sequence (human) | |

1. (canceled)

2. An isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of SEQ ID NO: 62, an HCDR2 comprising the amino acid sequence of SEQ ID NO: 5, and an HCDR3 comprising the amino acid sequence of SEQ ID NO: 6; and a light chain variable region (VL) comprising a light chain complementarity determining region 1 (LCDR1) comprising the amino acid sequence of SEQ ID NO: 7, an LCDR2 comprising the amino acid sequence of SEQ ID NO: 8, and an LCDR3 comprising the amino acid sequence of SEQ ID NO: 9.

3. (canceled)

4. An isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of SEQ ID NO: 62 and an HCDR3 comprising the amino acid sequence of SEQ ID NO: 6; and a light chain variable region (VL) comprising a light chain complementarity determining region 1 (LCDR1) comprising the amino acid sequence of SEQ ID NO: 7, wherein the VH of the antibody is at least 95% identical to the amino acid sequence of SEQ ID NO: 68 and wherein the VL of the antibody is at least 95% identical to the amino acid sequence of SEQ ID NO: 70.

5. An isolated antibody that specifically binds to protein arginine deiminase 4 (PAD4), wherein the antibody comprises a heavy chain variable region (VH) comprising a heavy chain complementarity determining region 1 (HCDR1) comprising the amino acid sequence of SEQ ID NO: 62, an HCDR2 comprising the amino acid sequence of SEQ ID NO: 5, and an HCDR3 comprising the amino acid sequence of SEQ ID NO: 6; and a light chain variable region (VL) comprising a light chain complementarity determining region 1 (LCDR1) comprising the amino acid sequence of SEQ ID NO: 7, an LCDR2 comprising the amino acid sequence of SEQ ID NO: 8, and an LCDR3 comprising the amino acid sequence of SEQ ID NO: 9, wherein the VH of the antibody is at least 95% identical to the amino acid sequence of SEQ ID NO: 68.

6. (canceled)

7. The isolated antibody of claim 2, wherein the VL of the antibody is at least 95% identical to the amino acid sequence of SEQ ID NO: 70.

8. The isolated antibody of claim 5, wherein the heavy chain variable region comprises a glycine at Kabat position 94 (which corresponds to position 98 of SEQ ID NO: 68).

9. The isolated antibody of claim 2, wherein the antibody comprises a VH comprising the amino acid sequence of SEQ ID NO: 68.

10. The isolated antibody of claim 2, wherein the antibody comprises a VL comprising the amino acid sequence of SEQ ID NO: 70.

11. An isolated antibody that specifically binds to protein arginine deiminase 4 (PAD4), wherein the antibody comprises a VH comprising the amino acid sequence of SEQ ID NO: 68 and a VL comprising the amino acid sequence of SEQ ID NO: 70.

12. (canceled)

13. The isolated antibody of claim 2, which is an IgA, IgG, or IgM antibody.

14. The isolated antibody of claim 2, which is a human IgG1, IgG2, IgG3, or IgG4 antibody.

15. The isolated antibody of claim 14, wherein the antibody comprises a wild-type, human IgG1, IgG2, or IgG4 heavy chain constant region.

16. The isolated antibody of claim 2, wherein the antibody comprises a human IgG1 or IgG2 heavy chain constant region comprising an amino acid sequence at least 95% identical to the amino acid sequence of any one of SEQ ID Nos: 174, 176, 178, 180, 182, 184, 186, 188, 190, or 192.

17. The isolated antibody of claim 2, wherein the antibody comprises a light chain constant region comprising an amino acid sequence at least 95% identical to the amino acid sequence of SEQ ID NO: 194.

18. The isolated antibody of claim 2, wherein the antibody comprises a human IgG1 or IgG2 heavy chain constant region comprising an amino acid sequence modified by 1-5 amino acid substitutions compared to the amino acid sequence of any one of SEQ ID NOs: 174, 176, 178, 180, 182, 184, 186, 188, 190; or 192.

19. The isolated antibody of claim 2, wherein the antibody comprises a light chain constant region comprising an amino acid sequence modified by 1-5 amino acid substitutions compared to the amino acid sequence of SEQ ID NO: 194.

20. The isolated antibody of claim 2, wherein the antibody comprises a human IgG1 or IgG2 heavy chain constant region comprising the amino acid sequence of any one of SEQ ID NOs: 174, 176, 178, 180, 182, 184, 186, 188, 190, or 192.

21. The isolated antibody of claim 2, wherein the antibody comprises a light chain constant region comprising the amino acid sequence of SEQ ID NO: 194.

22. The isolated antibody of claim 2, which is a full-length antibody.

23. The isolated antibody of claim 2, which is a human IgG antibody lacking a C-terminal lysine in the heavy chain constant region.

24. The isolated antibody of claim 2, which is an antibody fragment.

25. An isolated antibody that specifically binds to protein arginine deiminase 4 (PAD4), wherein the antibody comprises a heavy chain (HC) comprising the amino acid sequence of SEQ ID NO: 68 followed by the amino acid sequence of SEQ ID NO: 178 and a light chain (LC) comprising the amino acid sequence of SEQ ID NO: 70 followed by the amino acid sequence of SEQ ID NO: 194.

26. An isolated antibody that specifically binds to protein arginine deiminase 4 (PAD4), wherein the antibody comprises a heavy chain (HC) comprising the amino acid sequence of SEQ ID NO: 68 followed by the amino acid sequence of SEQ ID NO: 180 and a light chain (LC) comprising the amino acid sequence of SEQ ID NO: 70 followed by the amino acid sequence of SEQ ID NO: 194.

27. An isolated antibody that specifically binds to protein arginine deiminase 4 (PAD4), wherein the antibody comprises an HC comprising the amino acid sequence of SEQ ID NO: 196 and a light chain comprising the amino acid sequence of SEQ ID NO: 200.

28. An isolated antibody that specifically binds to protein arginine deiminase 4 (PAD4), wherein the antibody comprises an HC comprising the amino acid sequence of SEQ ID NO: 198 and a light chain comprising the amino acid sequence of SEQ ID NO: 200.

29. The isolated antibody of claim 2, which is a bispecific or multispecific antibody, or which is conjugated covalently or noncovalently to at least one other molecule.

30. The isolated antibody of claim 29, wherein the antibody is conjugated covalently or noncovalently to at least one other molecule, wherein the at least one other molecule comprises a detection label and/or a drug.

31. The isolated antibody of claim 2, wherein the antibody does not bind to human protein arginine deiminase 2 (PAD2).

32. The isolated antibody of claim 2, wherein the antibody has one or more of the following properties:
(a) dose-dependently inhibits PAD4 conversion of arginine to citrulline in peptide substrate (SHQESTRGKSKGKAAAAA; SEQ ID NO: 232) treated in vitro with 13.5 nM to 108 nM recombinant human PAD4 with an IC50 of 0.1-10 nM as determined using ELISA;
(b) specifically binds to PAD4 with a K_{D} from 0.05 nM to 0.5 nM as determined by surface plasmon resonance (SPR) (i) at 37° C. in the presence of 1 mM calcium chloride and (ii) at 37° C. in the absence of added Ca²⁺ and in the presence of 2 mM EDTA;
(c) an ECM score of less than 50;
(d) is less immunogenic than Campath® (alemtuzumab), Rituxan® (rituximab), and Zenapax® (daclizumab), as measured with the Epivax antibody immunogenicity scale in an in silico immunogenicity assay;
(e) reduces the amount of extracellular citrullinated histone H3 and reduces secretion of GM-CSF or gene expression of GM-CSF in LPS-stimulated human blood monocytes compared to an isotype control antibody; and
(f) decreases citrullination of ITIH4 and/or PRG4 in joint tissue.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. A pharmaceutical composition comprising an antibody of claim 2 and a pharmaceutically acceptable carrier.

47. An isolated nucleic acid or set of two or more nucleic acids encoding the antibody of claim 2.

48. An isolated vector comprising one or more nucleic acids encoding the heavy chain and the light chain of the antibody of claim 2.

49. An isolated host cell comprising the nucleic acid of claim 47.

50. A method of producing an antibody that specifically binds to PAD4 comprising culturing the host cell of claim 49 under conditions suitable for the expression of the antibody, optionally further comprising recovering the antibody from the host cell.

51. (canceled)

52. (canceled)

53. (canceled)

54. (canceled)

55. (canceled)

56. (canceled)

57. A method of treating an autoimmune disorder in a subject in need thereof, comprising administering to the subject an effective amount of the isolated antibody of claim 2.

58. The method of claim 57, wherein the autoimmune disorder is rheumatoid arthritis (RA), lupus, systemic lupus erythematosus (SLE), lupus nephritis, vasculitis, ANCA-associated vasculitis, thrombosis, venous thrombosis, inflammatory bowel disease (IBD), ulcerative colitis, or Crohn's disease.

59. The method of claim 57, wherein the method comprises administering at least one further therapeutic agent, optionally wherein the at least one further therapeutic agent is one or more of methotrexate, adalimumab, etanercept, infliximab, hydroxychloroquine, sulfasalazine, leflunomide, abatacept, anakinra, certolizumab, golimumab, rituximab, sarilumab, tocilizumab, baricitinib, tofacitinib, or upadacitinib.

60. (canceled)

61. (canceled)

62. A method of treating a subject at risk of developing RA, comprising administering to the subject an effective amount of the isolated antibody of claim 2.

63. The method of claim 62, wherein the subject has one or more of the following conditions:
a. At least one first-degree relative with RA;
b. Presence of anti-citrullinated protein antibodies (ACPA) in serum;
c. Presence of rheumatoid factor (RF) in serum;
d. Arthralgia in at least one joint;
e. Presence of inflammation in at least one joint observed by ultrasound or magnetic resonance imaging (MRI); and
f. Undifferentiated arthritis.

64. The method of claim 62, wherein the subject is in remission.

65. The method of claim 62, wherein the treatment is for delaying onset of RA.

66. (canceled)

67. (canceled)

68. A method of inhibiting NETosis or METosis in a subject, comprising administering to the subject an effective amount of the isolated antibody of claim 2.

69. A method of inhibiting NETosis in a subject, the method comprising administering to the subject an effective amount of the isolated antibody of claim 2.

70. A method of inhibiting METosis in a subject, the method comprising administering to the subject an effective amount of the isolated antibody of claim 2.

71. An in vitro method of inhibiting NETosis or METosis in a biological sample, comprising administering an effective amount of the isolated antibody of claim 2 to the biological sample.

72. A method of inhibiting citrullination in a subject, the method comprising administering to the subject an effective amount of the isolated antibody of claim 2.

73. An in vitro method of inhibiting citrullination in a biological sample, the method comprising administering an effective amount of the isolated antibody of claim 2 to the biological sample.

74. A method of treating an autoimmune disorder in a subject in need thereof, comprising administering to the subject an effective amount of the isolated antibody of claim 5.

75. The method of claim 74, wherein the autoimmune disorder is rheumatoid arthritis (RA), lupus, systemic lupus erythematosus (SLE), lupus nephritis, vasculitis, ANCA-associated vasculitis, thrombosis, venous thrombosis, inflammatory bowel disease (IBD), ulcerative colitis, or Crohn's disease.

76. The method of claim 74, wherein the method comprises administering at least one further therapeutic agent, optionally wherein the at least one further therapeutic agent is one or more of methotrexate, adalimumab, etanercept, infliximab, hydroxychloroquine, sulfasalazine, leflunomide, abatacept, anakinra, certolizumab, golimumab, rituximab, sarilumab, tocilizumab, baricitinib, tofacitinib, or upadacitinib.

77. A method of treating rheumatoid arthritis (RA) in a subject in need thereof, comprising administering to the subject an effective amount of the isolated antibody of claim 2.

78. A method of treating inflammatory bowel disease (IBD) in a subject in need thereof, comprising administering to the subject an effective amount of the isolated antibody of claim 2.

79. The method of claim 78, wherein the IBD is ulcerative colitis or Crohn's disease.

80. A method of treating a subject at risk of developing RA, comprising administering to the subject an effective amount of the isolated antibody of claim 5.

81. The method of claim 80, wherein the subject has one or more of the following conditions:
a. At least one first-degree relative with RA;
b. Presence of anti-citrullinated protein antibodies (ACPA) in serum;
c. Presence of rheumatoid factor (RF) in serum;
d. Arthralgia in at least one joint;
e. Presence of inflammation in at least one joint observed by ultrasound or magnetic resonance imaging (MM); and
f. Undifferentiated arthritis.

82. The method of claim 80, wherein the subject is in remission.

83. The method of claim 80, wherein the treatment is for delaying onset of RA.

84. A method of inhibiting NETosis or METosis in a subject, comprising administering to the subject an effective amount of the isolated antibody of claim 5.

85. A method of inhibiting NETosis in a subject, the method comprising administering to the subject an effective amount of the isolated antibody of claim 5.

86. A method of inhibiting METosis in a subject, the method comprising administering to the subject an effective amount of the isolated antibody of claim 5.

87. An in vitro method of inhibiting NETosis or METosis in a biological sample, comprising administering an effective amount of the isolated antibody of claim 5 to the biological sample.

88. A method of inhibiting citrullination in a subject, the method comprising administering to the subject an effective amount of the isolated antibody of claim 5.

89. An in vitro method of inhibiting citrullination in a biological sample, the method comprising administering an effective amount of the isolated antibody of claim 5 to the biological sample.

90. A method of treating rheumatoid arthritis (RA) in a subject in need thereof, comprising administering to the subject an effective amount of the isolated antibody of claim 5.

91. A method of treating inflammatory bowel disease (IBD) in a subject in need thereof, comprising administering to the subject an effective amount of the isolated antibody of claim 5.

92. The method of claim 91, wherein the IBD is ulcerative colitis or Crohn's disease.

93. The isolated antibody of claim 2, wherein the VH of the antibody is at least 95% identical to the amino acid sequence of SEQ ID NO: 68 and wherein the VL of the antibody is at least 95% identical to the amino acid sequence of SEQ ID NO: 70.

94. The isolated antibody of claim 5, which is an IgA, IgG, or IgM antibody.

95. The isolated antibody of claim 5, which is a human IgG1, IgG2, IgG3, or IgG4 antibody.

96. The isolated antibody of claim 95, wherein the antibody comprises a wild-type, human IgG1, IgG2, or IgG4 heavy chain constant region.

97. The isolated antibody of claim 5, wherein the antibody comprises a human IgG1 or IgG2 heavy chain constant region comprising an amino acid sequence at least 95% identical to the amino acid sequence of any one of SEQ ID Nos: 174, 176, 178, 180, 182, 184, 186, 188, 190, or 192.

98. The isolated antibody of claim 5, wherein the antibody comprises a light chain constant region comprising an amino acid sequence at least 95% identical to the amino acid sequence of SEQ ID NO: 194.

99. The isolated antibody of claim 5, wherein the antibody comprises a human IgG1 or IgG2 heavy chain constant region comprising an amino acid sequence modified by 1-5 amino acid substitutions compared to the amino acid sequence of any one of SEQ ID NOs: 174, 176, 178, 180, 182, 184, 186, 188, 190; or 192.

100. The isolated antibody of claim 5, wherein the antibody comprises a light chain constant region comprising an amino acid sequence modified by 1-5 amino acid substitutions compared to the amino acid sequence of SEQ ID NO: 194.

101. The isolated antibody of claim 5, wherein the antibody comprises a human IgG1 or IgG2 heavy chain constant region comprising the amino acid sequence of any one of SEQ ID NOs: 174, 176, 178, 180, 182, 184, 186, 188, 190, or 192.

102. The isolated antibody of claim 5, wherein the antibody comprises a light chain constant region comprising the amino acid sequence of SEQ ID NO: 194.

103. The isolated antibody of claim 5, which is a full-length antibody.

104. The isolated antibody of claim 5, which is a human IgG antibody lacking a C-terminal lysine in the heavy chain constant region.

105. The isolated antibody of claim 5, which is an antibody fragment.

106. The isolated antibody of claim 5, wherein the antibody has one or more of the following properties:
(a) dose-dependently inhibits PAD4 conversion of arginine to citrulline in peptide substrate (SHQESTRGKSKGKAAAAA; SEQ ID NO: 232) treated in vitro with 13.5 nM to 108 nM recombinant human PAD4 with an IC50 of 0.1-10 nM as determined using ELISA;
(b) specifically binds to PAD4 with a K_{D} from 0.05 nM to 0.5 nM as determined by surface plasmon resonance (SPR) (i) at 37° C. in the presence of 1 mM calcium chloride and (ii) at 37° C. in the absence of added Ca²⁺ and in the presence of 2 mM EDTA;
(c) an ECM score of less than 50;
(d) is less immunogenic than Campath® (alemtuzumab), Rituxan® (rituximab), and Zenapax® (daclizumab), as measured with the Epivax antibody immunogenicity scale in an in silico immunogenicity assay;
(e) reduces the amount of extracellular citrullinated histone H3 and reduces secretion of GM-CSF or gene expression of GM-CSF in LPS-stimulated human blood monocytes compared to an isotype control antibody; and
(f) decreases citrullination of ITIH4 and/or PRG4 in joint tissue.
